(19) United States
(12) Patent Application Publication
(10) Pub. No.: US 2022/0380278 A1
(43) Pub. Date: Dec. 1, 2022

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Satomi TASAKI, Tokyo (JP); Kazuki NISHIMURA, Tokyo (JP); Yuki NAKANO, Tokyo (JP)

(73) Assignee: IDEMITSU KOSAN CO.,LTD., Tokyo (JP)

(21) Appl. No.: 17/642,661

(22) PCT Filed: Sep. 11, 2020
(86) PCT No.: PCT/JP2020/034605

§ 371 Date: Mar. 11, 2022

(30) Foreign Application Priority Data
Sep. 13, 2019 (JP) 2019-167636
Nov. 26, 2019 (JP) 2019-213374
Dec. 27, 2019 (JP) 2019-239937
Dec. 27, 2019 (JP) 2019-239946

(51) Int. Cl. C07C 15/38; C07D 307/91; C07C 15/28; C07C 15/30; C07D 333/76; C07D 311/82; C07F 7/08; C07D 405/10; C07D 407/10; C07C 13/72; C07C 211/54; C07C 255/51; C07D 487/06; C07D 491/22; C07D 491/06; C07D 487/16; C07D 495/22; C07D 519/00; C07F 5/02; C07D 493/04; C07D 495/04; H01L 51/00

(52) U.S. Cl. CPC C07C 15/38; C07D 307/91; C07C 15/28; C07C 15/30; C07D 333/76; C07D 311/82; C07F 7/0812; C07D 405/10; C07D 407/10; C07C 13/72; C07C 211/54; C07C 255/51; C07D 487/06; C07D 491/22; C07D 491/06; C07D 487/16; C07D 495/22; C07D 519/00; C07F 5/027; C07D 493/04; C07D 495/04; C07F 7/081; H01L 51/0094; H01L 51/008; H01L 51/0052; H01L 51/0054; H01L 51/0055; H01L 51/0056; H01L 51/0059; H01L 51/006; H01L 51/0064; H01L 51/0071; H01L 51/0072; H01L 51/0073; H01L 51/0074; C07B 2200/05; H01L 51/5024

(57) ABSTRACT

An organic electroluminescence device includes an anode, a cathode, a first emitting layer, and a second emitting layer provided between the first emitting layer and the cathode, in which the first emitting layer includes, as a first host material, a first compound that includes at least one group represented by a formula (11) below, the first compound being represented by a formula (1) below, the second emitting layer includes a second compound represented by a formula (2) below as a second host material, and the first emitting layer is in direct contact with the second emitting layer. L₁₀₁ is a single bond or a substituted or unsubstituted arylene group having 6 to 50 ring carbon atoms. Ar₁₀₁ is a substituted or unsubstituted aryl group having 6 to 50 ring carbon atoms. mx is 0, 1, 2, 3, 4, or 5.

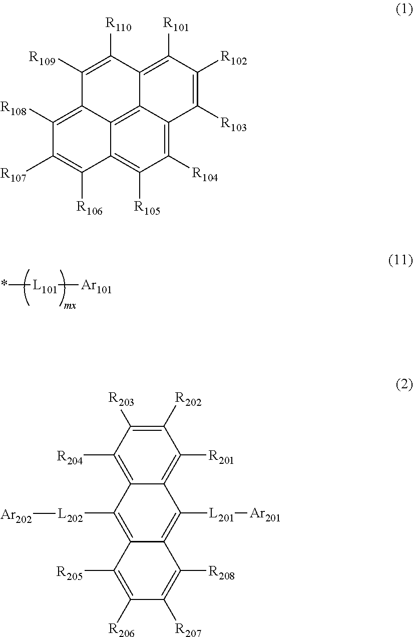

### TECHNICAL FIELD

The present invention relates to an organic electroluminescence device and an electronic device.

### BACKGROUND ART

An organic electroluminescence device (hereinafter, occasionally referred to as “organic EL device”) has found its application in a full-color display for mobile phones, televisions and the like. When a voltage is applied to an organic EL device, holes and electrons are injected from an anode and a cathode, respectively, into an emitting layer. The injected holes and electrons are recombined in the emitting layer to form excitons. Specifically, according to the electron spin statistics theory, singlet excitons and triplet excitons are generated at a ratio of 25%:75%.

Various studies have been made for compounds to be used for the organic EL device in order to enhance the performance of the organic EL device (e.g., see Patent Literatures 1 to 6). The performance of the organic EL device is evaluable in terms of, for instance, luminance, emission wavelength, chromaticity, emission efficiency, drive voltage, and lifetime.

### CITATION LIST

### Patent Literature(s)

Patent Literature 1: JP 2013-157552 A
Patent Literature 2: International Publication No. WO2004/018587
Patent Literature 3: International Publication No. WO2005/115950
Patent Literature 4: International Publication No. WO2011/077691
Patent Literature 5: JP 2018-125504 A
Patent Literature 6: US Patent Application Publication No. 2019/280209

### SUMMARY OF THE INVENTION

### Problems to be Solved by the Invention

An object of the invention is to provide an organic electroluminescence device with enhanced performance. Another object of the invention is to provide an organic electroluminescence device with improved luminous efficiency and an electronic device including the organic electroluminescence device.

### Means for Solving the Problems

According to an aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11) below, the first compound being represented by a formula (1) below, the second emitting layer contains a second compound represented by a formula (2) below as a second host material, and the first emitting layer is in direct contact with the second emitting layer.

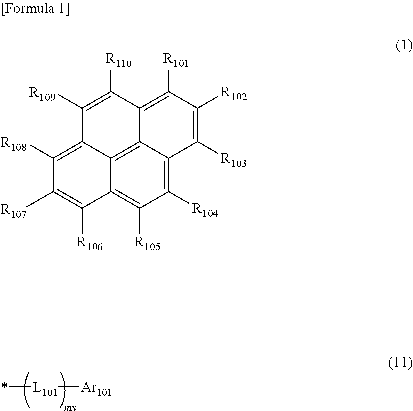

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a single bond, a substituted or unsubstituted arylene group having 6 to 50 ring carbon atoms, or a substituted or unsubstituted divalent heterocyclic group having 5 to 50 ring atoms;

Ar₁₀₁ is a substituted or unsubstituted aryl group having 6 to 50 ring carbon atoms, or a substituted or unsubstituted heterocyclic group having 5 to 50 ring atoms;

mx is 0, 1, 2, 3, 4, or 5;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

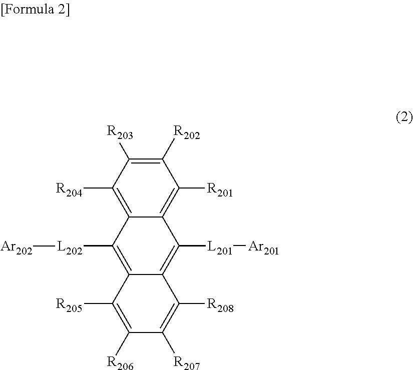

In the formula (2):

R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the first compound represented by the formula (1) and the second compound represented by the formula (2), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₈₀₁ are present, the plurality of R₈₀₁ are mutually the same or different; and

when a plurality of R₈₀₂ are present, the plurality of R₈₀₂ are mutually the same or different.

According to another aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11) below, the first compound being represented by a formula (1) below, the second emitting layer contains a second compound represented by a formula (2) below as a second host material, and the first emitting layer is in direct contact with the second emitting layer.

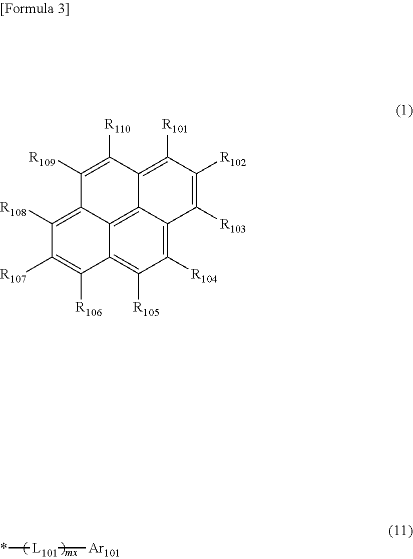

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a single bond, or a substituted or unsubstituted arylene group having 6 to 50 ring carbon atoms;

Ar₁₀₁ is a substituted or unsubstituted aryl group having 6 to 50 ring carbon atoms;

mx is 0, 1, 2, 3, 4, or 5;

Ar₁₀₁ is not a substituted or unsubstituted pyrenyl group;

L₁₀₁ is not a substituted or unsubstituted pyrenylene group;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

According to still another aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least two groups each represented by a formula (11) below, the first compound being represented by a formula (1) below, the second emitting layer contains a second compound represented by a formula (2) below as a second host material, and the first emitting layer is in direct contact with the second emitting layer.

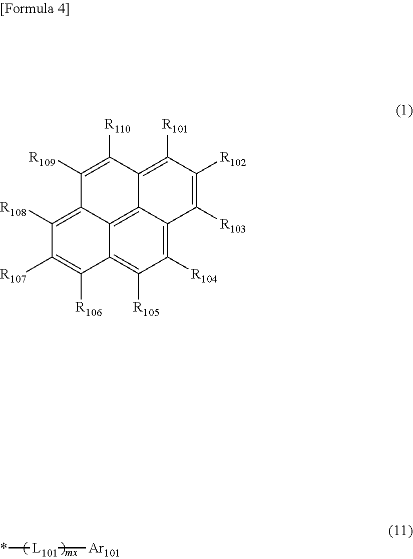

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, or a group represented by the formula (11);

two or more of R₁₀₁ to R₁₁₀ are each a group represented by the formula (11);

when a plurality of groups represented by the formula (11) are present, the plurality of groups represented by the formula (11) are mutually the same or different;

L₁₀₁ is a single bond;

Ar₁₀₁ is a substituted or unsubstituted heterocyclic group having 5 to 50 ring atoms;

mx is 0;

when two or more Ar₁₀₁ are present, the two or more Ar₁₀₁ are mutually the same or different; and

* in the formula (11) represents a bonding position to a pyrene ring in the formula (1).

According to a further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least two groups each represented by a formula (11) below, the first compound being represented by a formula (1) below, the second emitting layer contains a second compound represented by a formula (2) below as a second host material, and the first emitting layer is in direct contact with the second emitting layer.

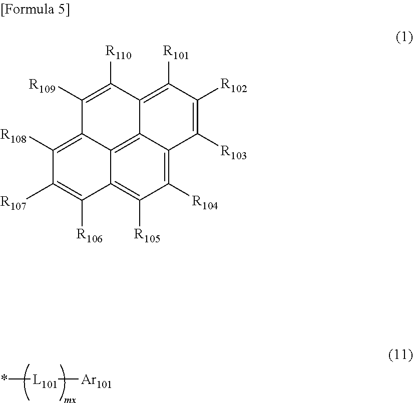

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a substituted or unsubstituted divalent heterocyclic group having 5 to 50 ring atoms;

Ar₁₀₁ is a substituted or unsubstituted heterocyclic group having 5 to 50 ring atoms;

mx is 1, 2, 3, 4 or 5;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11) below, the first compound being represented by a formula (1) below, the second emitting layer contains a second compound represented by a formula (2) below as a second host material, and the first emitting layer is in direct contact with the second emitting layer.

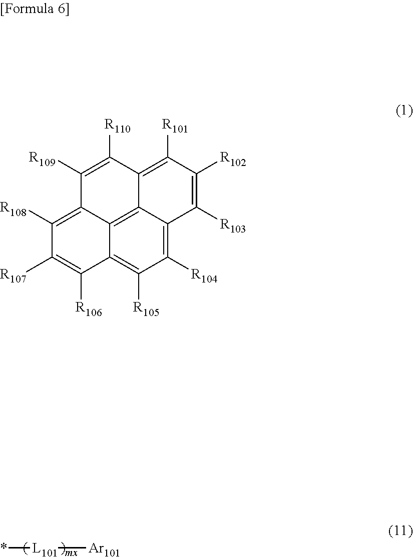

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

at least one of R₁₀₁ to R₁₁₀ is a substituted or unsubstituted heterocyclic group having 5 to 50 ring atoms;

when a plurality of groups represented by the formula (11) are present, the plurality of groups represented by the formula (11) are mutually the same or different;

L₁₀₁ is a single bond, or a substituted or unsubstituted arylene group having 6 to 50 ring carbon atoms;

Ar₁₀₁ is a substituted or unsubstituted aryl group having 6 to 50 ring carbon atoms;

mx is 0, 1, 2, 3, 4, or 5;

Ar₁₀₁ is not a substituted or unsubstituted pyrenyl group;

L₁₀₁ is not a substituted or unsubstituted pyrenylene group;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11) below, the first compound being represented by a formula (1) below, the second emitting layer contains a second compound represented by a formula (2) below as a second host material, and the first emitting layer is in direct contact with the second emitting layer.

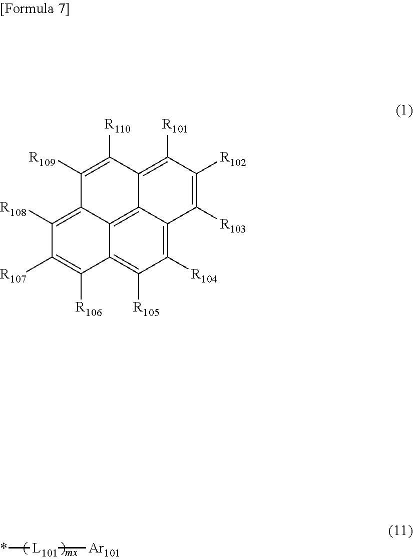

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a substituted or unsubstituted arylene group having 6 to 50 ring carbon atoms;

Ar₁₀₁ is a substituted or unsubstituted heterocyclic group having 5 to 50 ring atoms;

mx is 1, 2, 3, 4 or 5;

L₁₀₁ is not a substituted or unsubstituted pyrenylene group;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11) below, the first compound being represented by a formula (1) below, the second emitting layer contains a second compound represented by a formula (2) below as a second host material, and the first emitting layer is in direct contact with the second emitting layer.

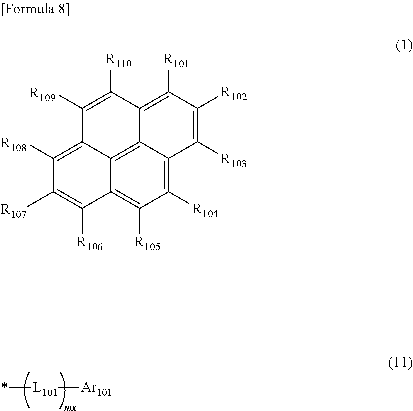

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a substituted or unsubstituted divalent heterocyclic group having 5 to 50 ring atoms;

Ar₁₀₁ is a substituted or unsubstituted aryl group having 6 to 50 ring carbon atoms;

mx is 1, 2, 3, 4 or 5;

Ar₁₀₁ is not a substituted or unsubstituted pyrenyl group;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11A) below, the first compound being represented by a formula (1) below, the second emitting layer contains, as a second host material, a second compound represented by the formula (2), and the first emitting layer is in direct contact with the second emitting layer.

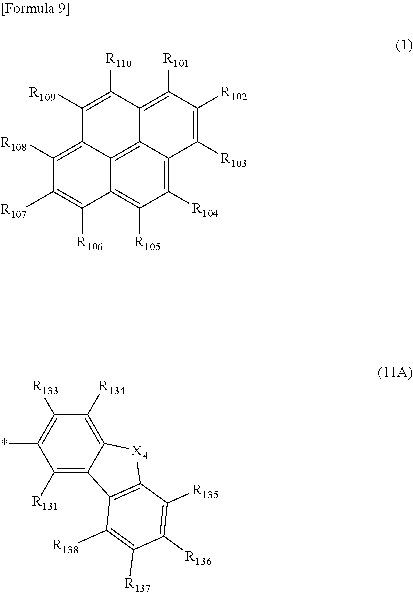

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11A);

at least one of R₁₀₁ to R₁₁₀ is a group represented by the formula (11A);

when a plurality of groups represented by the formula (11A) are present, the plurality of groups represented by the formula (11A) are mutually the same or different;

X_{A} is an oxygen atom or a sulfur atom;

at least one combination of adjacent two or more of R₁₃₁, R₁₃₃ and R₁₃₄ are mutually bonded to form a substituted or unsubstituted monocyclic ring, mutually bonded to form a substituted or unsubstituted fused ring, or not mutually bonded;

R₁₃₅ to R₁₃₈, and R₁₃₁, R₁₃₃ and R₁₃₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formula (11A) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11B) below, the first compound being represented by a formula (1) below, the second emitting layer contains, as a second host material, a second compound represented by the formula (2) and the first emitting layer is in direct contact with the second emitting layer.

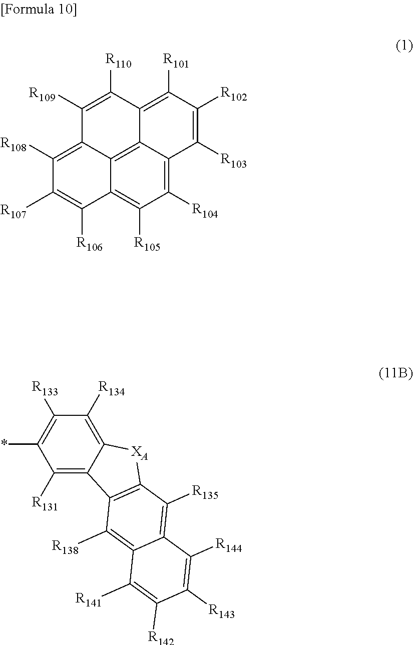

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11B);

at least one of R₁₀₁ to R₁₁₀ is a group represented by the formula (11B);

when a plurality of groups represented by the formula (11B) are present, the plurality of groups represented by the formula (11B) are mutually the same or different;

X_{A} is an oxygen atom or a sulfur atom;

at least one combination of adjacent two or more of R₁₃₁, R₁₃₃ to R₁₃₅, R₁₃₈ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃ to R₁₃₅, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

at least one of R₁₃₅, R₁₃₈ or R₁₄₁ to R₁₄₄ are a substituted or unsubstituted aryl group having 6 to 50 ring carbon atoms, or a substituted or unsubstituted heterocyclic group having 5 to 50 ring atoms; and

* in the formula (11B) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11C) below, the first compound being represented by a formula (1) below, the second emitting layer contains a second compound represented by a formula (2) below as a second host material, and the first emitting layer is in direct contact with the second emitting layer.

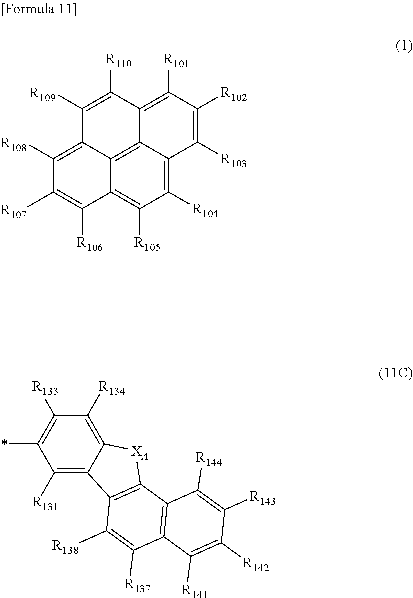

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11C);

at least one of R₁₀₁ to R₁₁₀ is a group represented by the formula (11C);

when a plurality of groups represented by the formula (11C) are present, the plurality of groups represented by the formula (11C) are mutually the same or different;

X_{A} is an oxygen atom or a sulfur atom;

at least one combination of adjacent two or more of R₁₃₁, R₁₃₃, R₁₃₄, R₁₃₇, R₁₃₈ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃, R₁₃₄, R₁₃₇, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formula (11C) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11D) below, the first compound being represented by a formula (1) below, the second emitting layer contains a second compound represented by a formula (2) below as a second host material, and the first emitting layer is in direct contact with the second emitting layer.

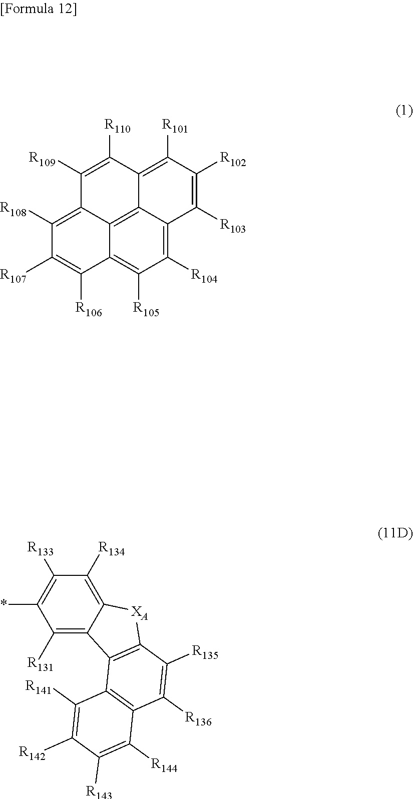

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11D);

at least one of R₁₀₁ to R₁₁₀ is a group represented by the formula (11D);

when a plurality of groups represented by the formula (11D) are present, the plurality of groups represented by the formula (11D) are mutually the same or different;

X_{A} is an oxygen atom or a sulfur atom;

at least one combination of adjacent two or more of R₁₃₁, R₁₃₃, R₁₃₄, R₁₃₅, R₁₃₆ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃, R₁₃₄, R₁₃₅, R₁₃₆ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formula (11D) represents a bonding position to a pyrene ring in the formula (1).

According to a still further aspect of the invention, there is provided an organic electroluminescence device including: an anode; a cathode; a first emitting layer provided between the anode and the cathode; and a second emitting layer provided between the first emitting layer and the cathode, in which the first emitting layer contains, as a first host material, a first compound that includes at least one group represented by a formula (11E) below, the first compound being represented by a formula (1) below, the second emitting layer contains, as a second host material, a second compound represented by a formula (2) below, and the first emitting layer is in direct contact with the second emitting layer.

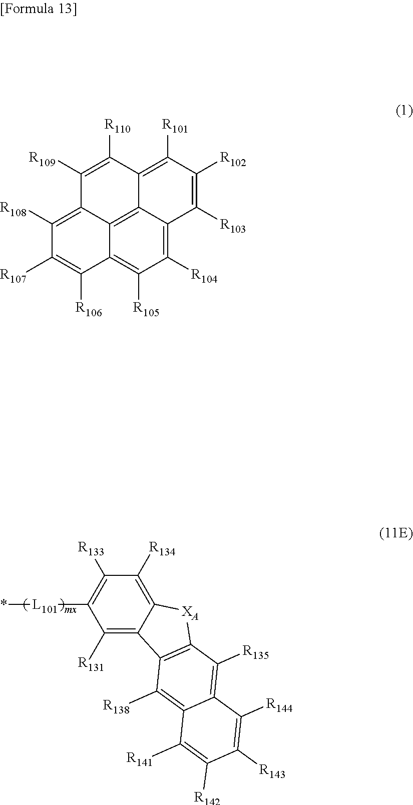

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11E);

at least one of R₁₀₁ to R₁₁₀ is a group represented by the formula (11E);

when a plurality of groups represented by the formula (11E) are present, the plurality of groups represented by the formula (11E) are mutually the same or different;

X_{A} is an oxygen atom or a sulfur atom;

at least one combination of adjacent two or more of R₁₃₁, R₁₃₃ to R₁₃₅, R₁₃₈ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃ to R₁₃₅, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

L₁₀₁ is a substituted or unsubstituted arylene group having 6 to 50 ring carbon atoms, or a substituted or unsubstituted divalent heterocyclic group having 5 to 50 ring atoms;

mx is 1, 2, 3, 4 or 5;

when two or more L₁₀₁ are present, the two or more L₁₀₁ are mutually the same or different; and

* in the formula (11E) represents a bonding position to a pyrene ring in the formula (1).

According to still further aspect of the invention, an electronic device including the organic electroluminescence device according to the above aspect of the invention is provided.

According to the above aspects of the invention, an organic electroluminescence device with enhanced performance can be provided. According to the above aspects of the invention, an organic electroluminescence device with improved luminous efficiency can be provided. According to the above aspects of the invention, an electronic device including the organic electroluminescence device can be provided.

### BRIEF DESCRIPTION OF DRAWING(S)

FIG. 1 schematically shows an exemplary arrangement of an organic electroluminescence device according to an exemplary embodiment of the invention.

### DESCRIPTION OF EMBODIMENTS

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and do not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B) below. (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A)

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, a phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from cyclic structures represented by formulae (TEMP-1) to (TEMP-15) below.

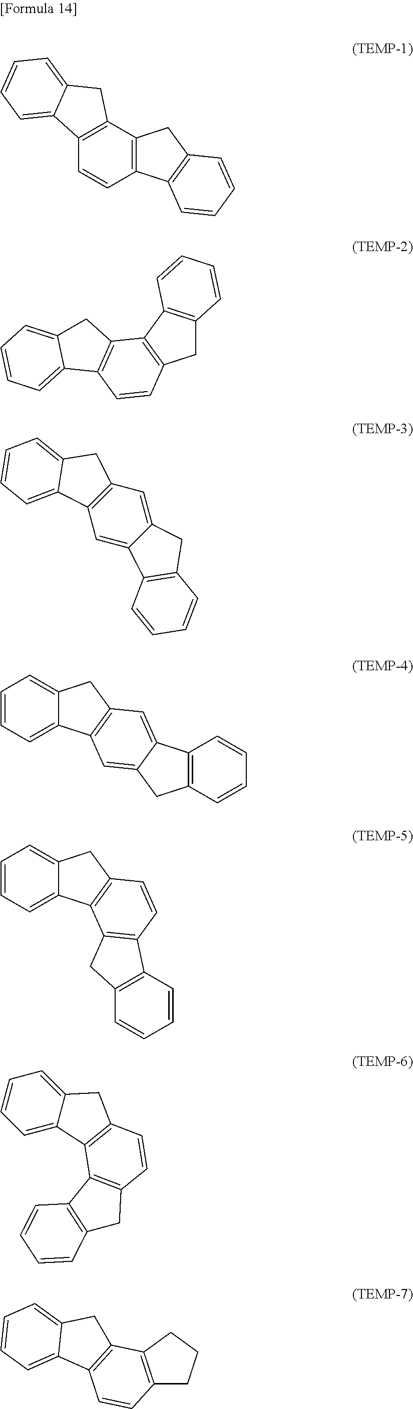

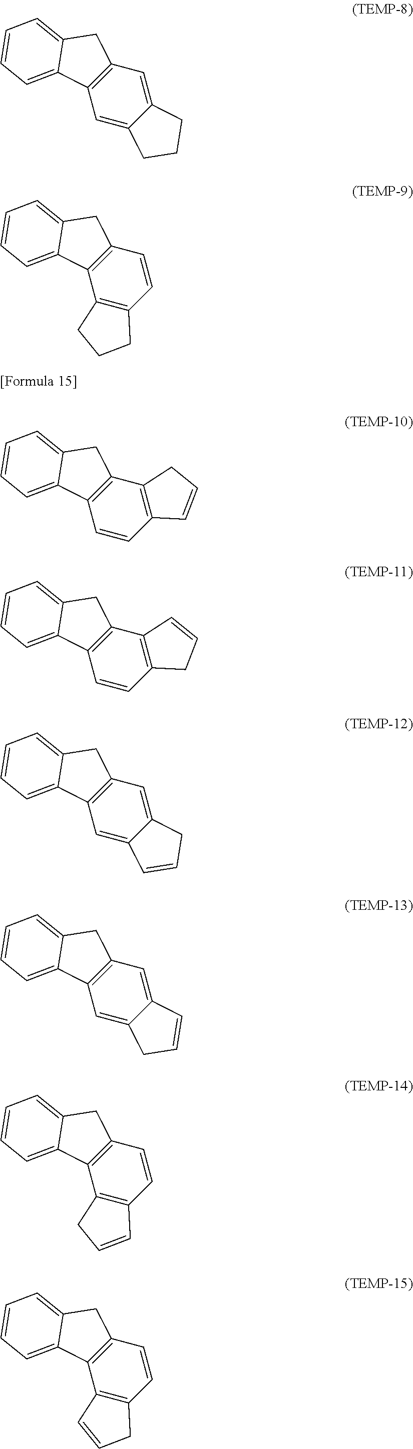

### Substituted Aryl Group (Specific Example Group G1B)

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of “unsubstituted heterocyclic group” and “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1)

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2)

furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3)

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

### Monovalent Heterocyclic Groups Derived by Removing One Hydrogen Atom from Cyclic Structures Represented by Formulae (TEMP-16) to (TEMP-33) (Specific Example Group G2A4)

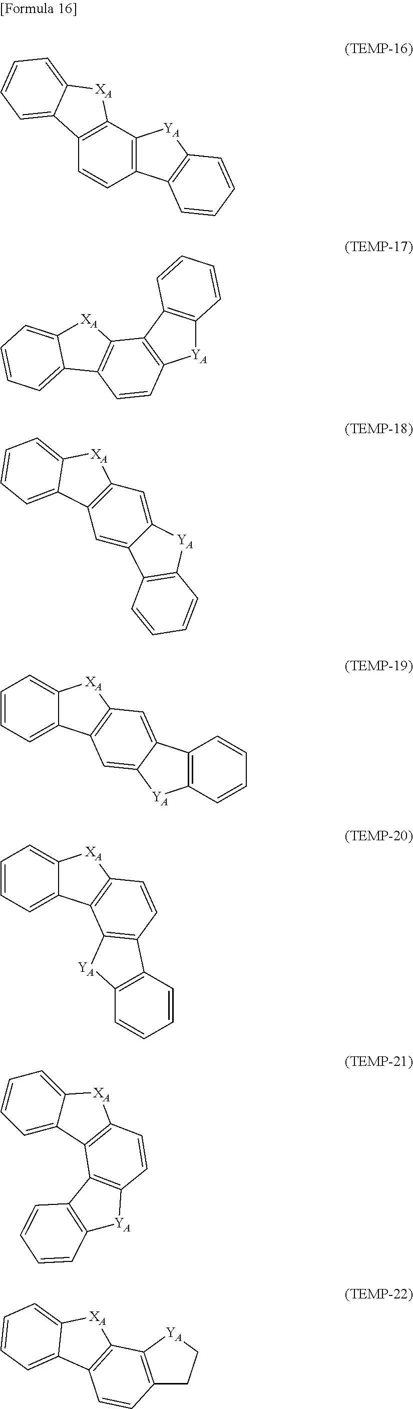

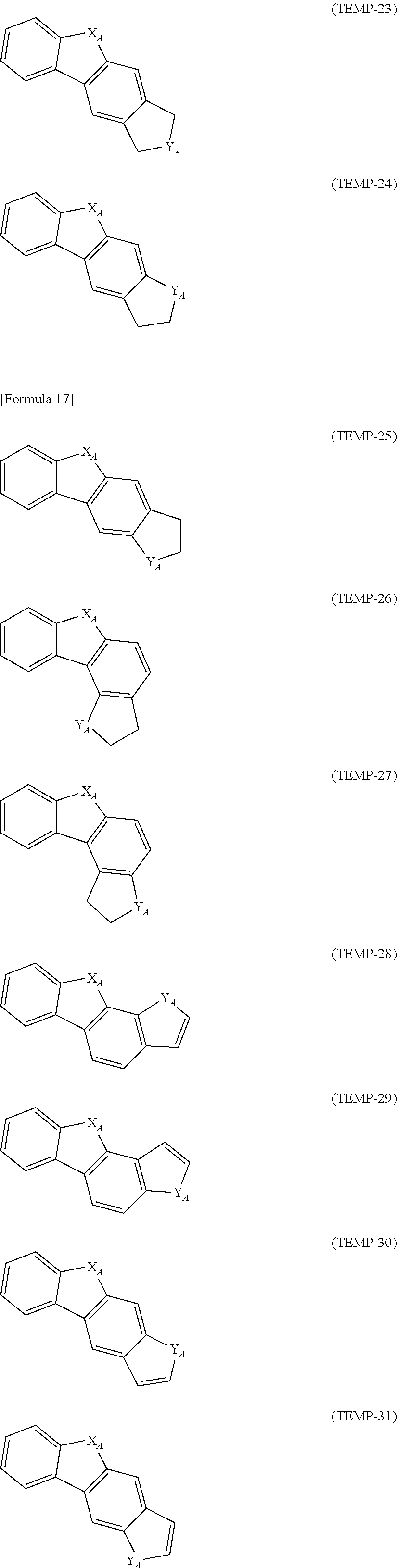

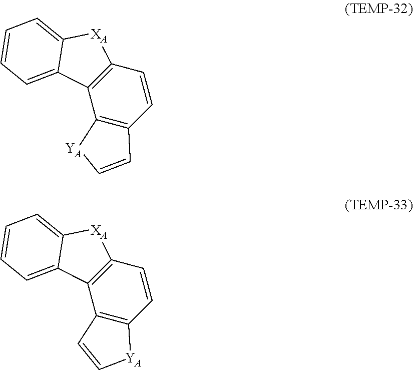

In the formulae (TEMP-16) to (TEMP-33), X_{A} and Y_{A} are each independently an oxygen atom, a sulfur atom, NH, or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1)

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2)

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3)

phenyldibenzothiophenyl group, methyldibenzothiophenyl group, t-butyldibenzothiophenyl group, and monovalent residue of spiro[9H-thioxanthene-9,9′-[9H]fluorene].

### Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4)

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of XA or YA in a form of NH, and a hydrogen atom of one of XA and YA in a form of a methylene group (CH2).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of “unsubstituted alkyl group” and “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A)

methyl group, ethyl group, n-propyl group, isopropyl group, n-butyl group, isobutyl group, s-butyl group, and t-butyl group

### Substituted Alkyl Group (Specific Example Group G3B)

heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of “unsubstituted alkenyl group” and “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A)

vinyl group, allyl group, 1-butenyl group, 2-butenyl group, and 3-butenyl group

### Substituted Alkenyl Group (Specific Example Group G4B)

1,3-butanedienyl group, 1-methylvinyl group, 1-methylallyl group, 1,1-dimethylallyl group, 2-methylallyl group, and 1,2-dimethylallyl group

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A): Ethynyl Group

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A)

cyclopropyl group, cyclobutyl group, cyclopentyl group, cyclohexyl group, 1-adamantyl group, 2-adamantyl group, 1-norbornyl group, and 2-norbornyl group

### Substituted Cycloalkyl Group (Specific Example Group G6B): 4-methylcyclohexyl Group

Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6),

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —Si(G1)(G1)(G1) are mutually the same or different;

a plurality of G2 in —Si(G1)(G2)(G2) are mutually the same or different;

a plurality of G1 in —Si(G1)(G1)(G2) are mutually the same or different;

a plurality of G2 in —Si(G2)(G2)(G2) are mutually the same or different;

a plurality of G3 in —Si(G3)(G3)(G3) are mutually the same or different; and

a plurality of G6 in —Si(G6)(G6)(G6) are mutually the same or different.

Group Represented by —O—(R₉₀₄)

Specific examples (specific example group G8) of a group represented by —O—(R₉₀₄) herein include: —O(G1); —O(G2); —O(G3); and —O(G6),

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6), where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6;

Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6),

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —N(G1)(G1) are mutually the same or different;

a plurality of G2 in —N(G2)(G2) are mutually the same or different;

a plurality of G3 in —N(G3)(G3) are mutually the same or different; and

a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

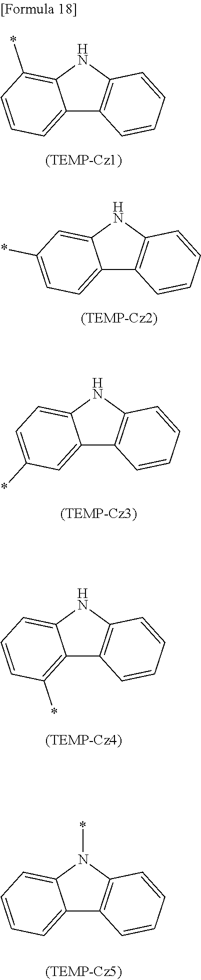 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

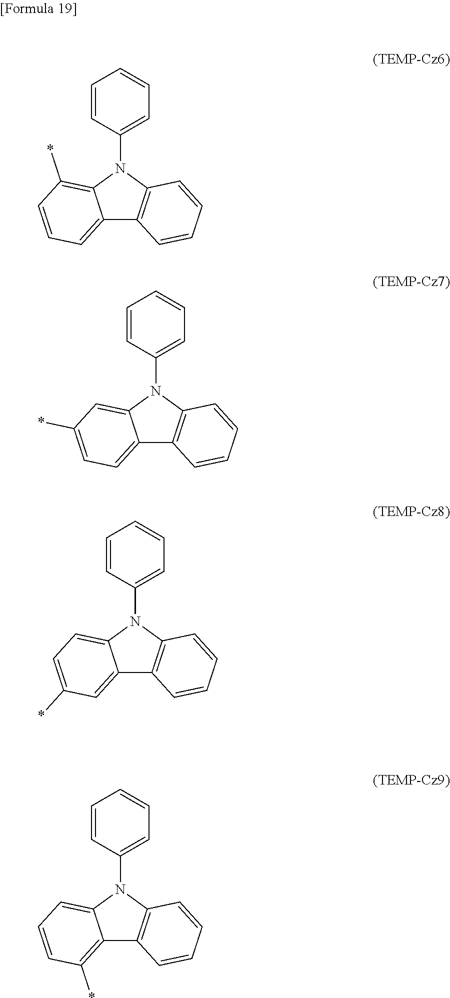 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

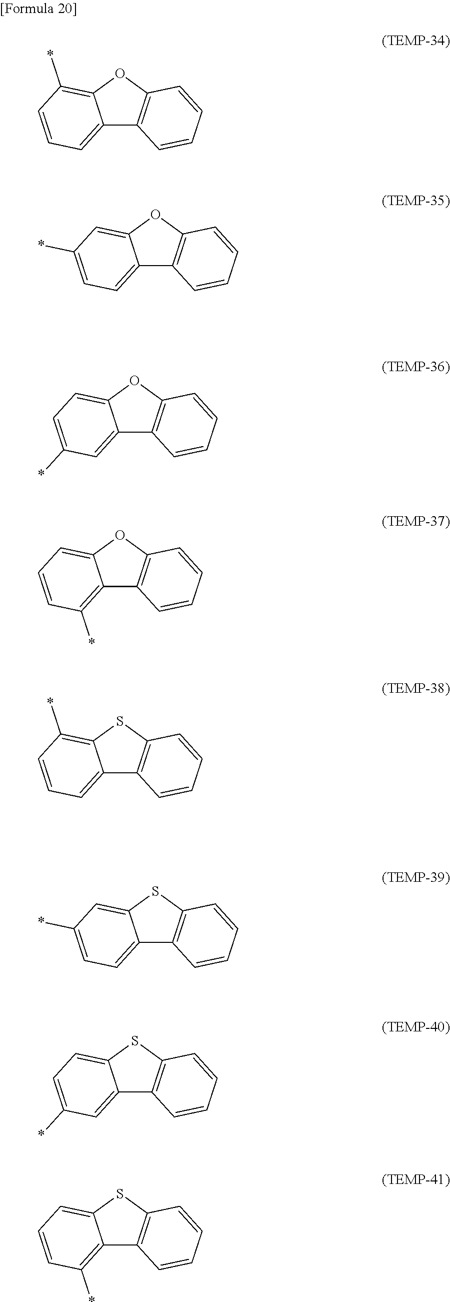

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

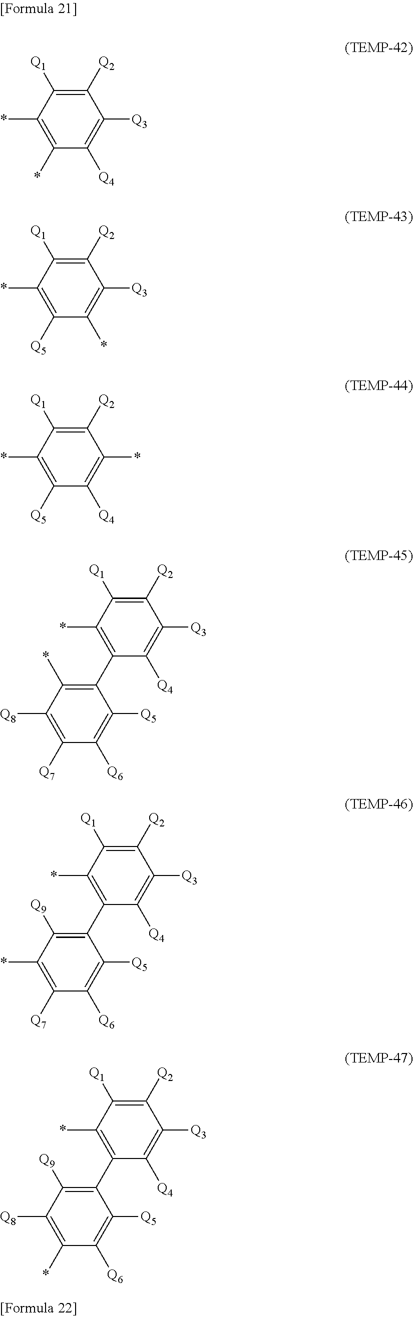

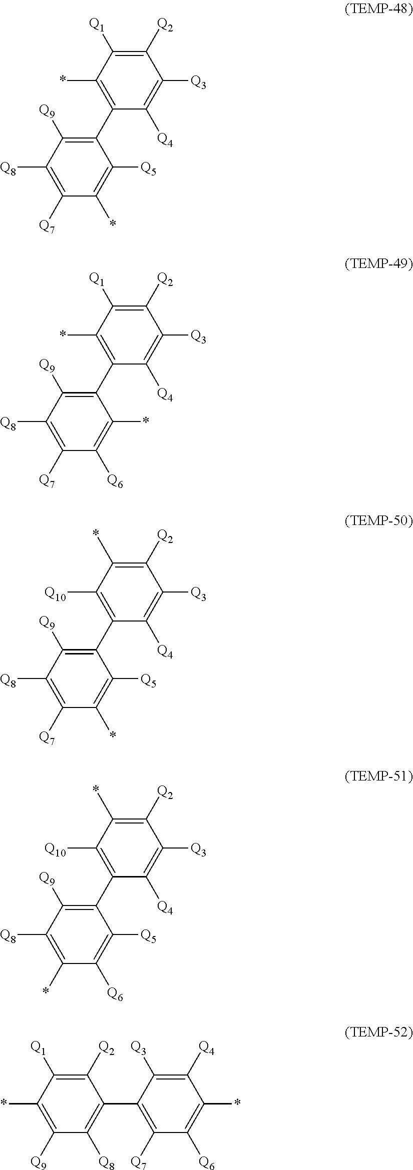

In the formulae (TEMP-42) to (TEMP-52), Q₁ to Q₁₀ are each independently a hydrogen atom or a substituent.

In the formulae (TEMP-42) to (TEMP-52), * represents a bonding position.

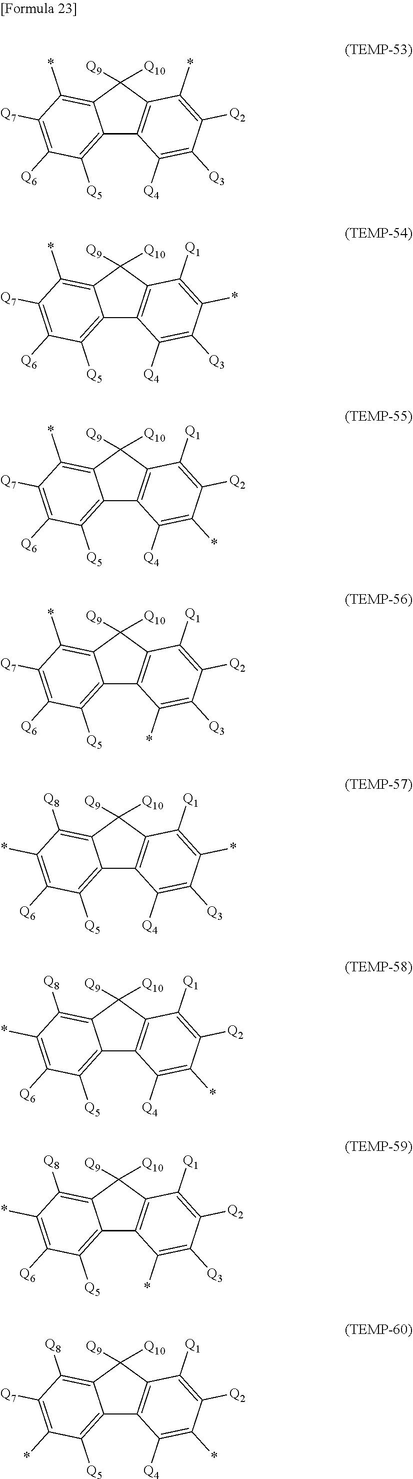

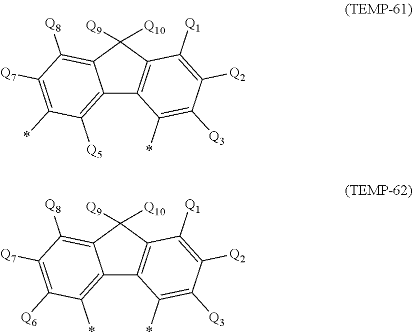

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

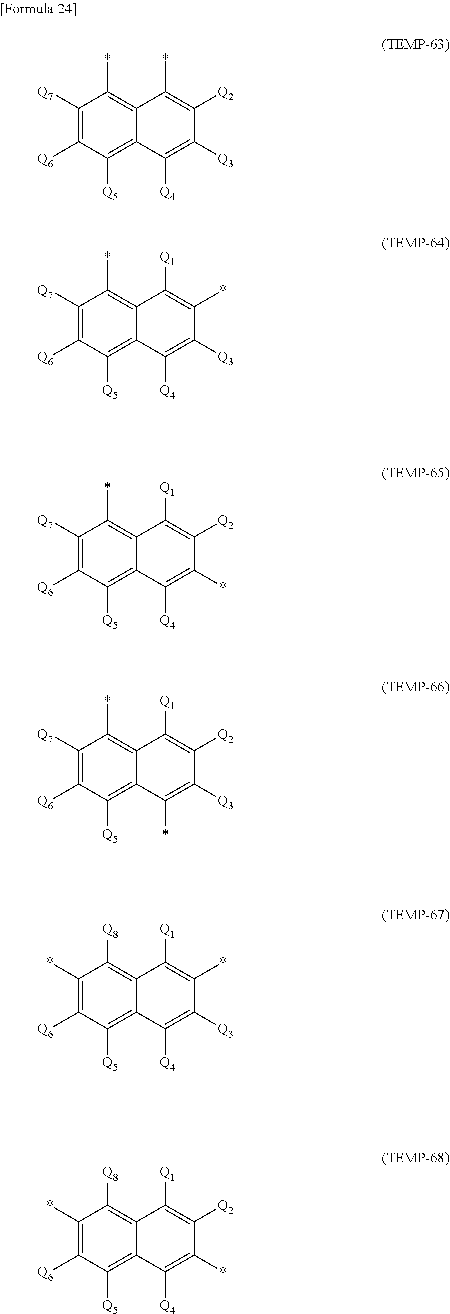

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

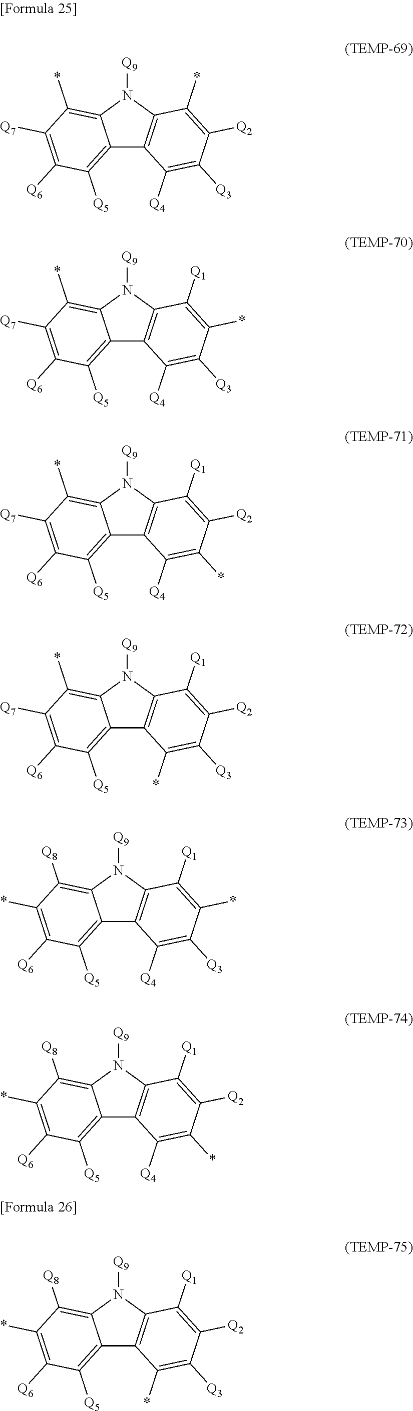

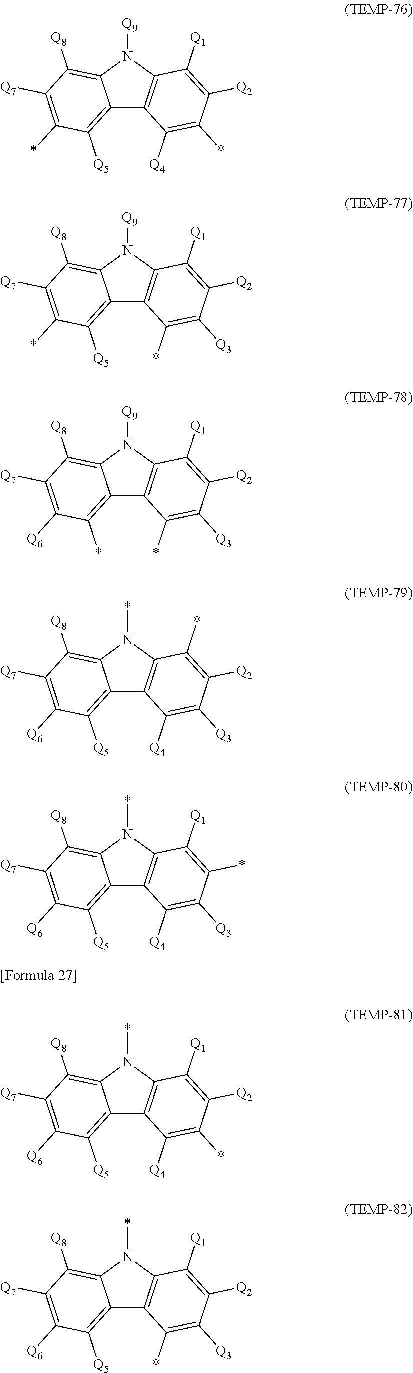

In the formulae (TEMP-69) to (TEMP-82), Q₁ to Q₉ are each independently a hydrogen atom or a substituent.

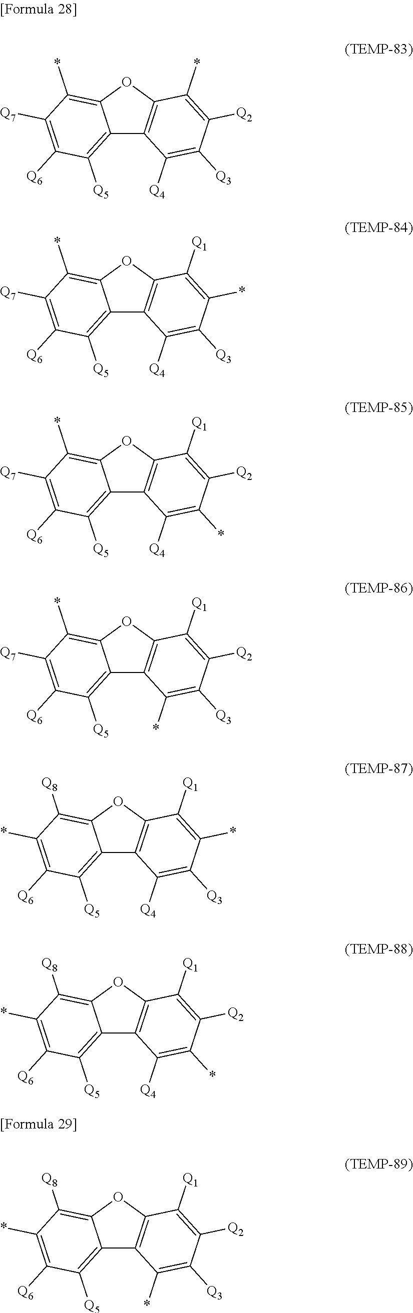

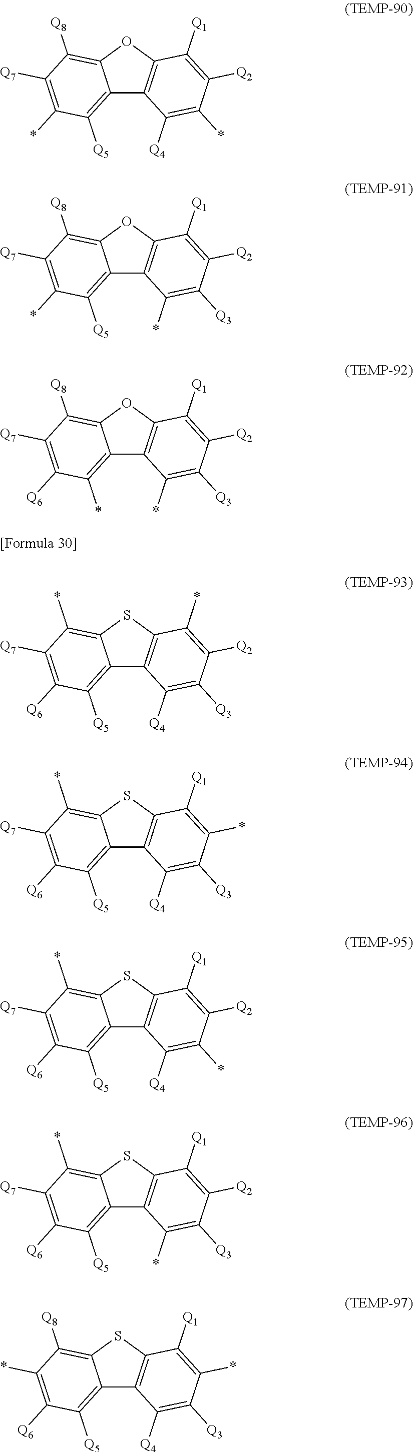

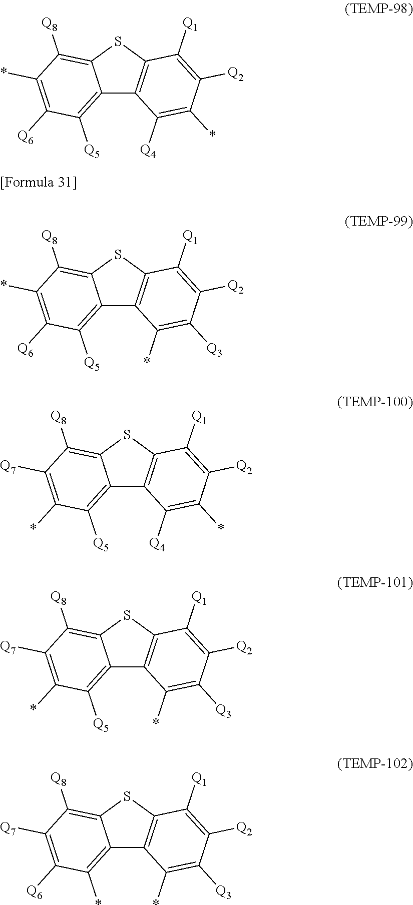

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

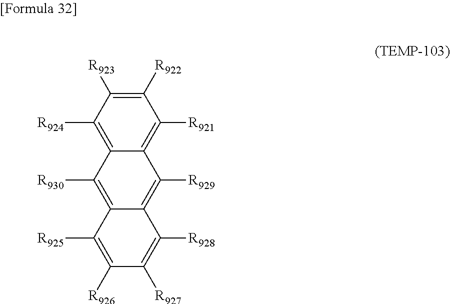

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

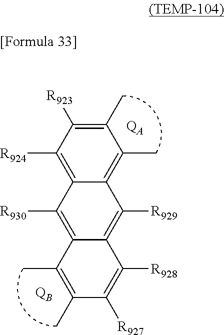

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

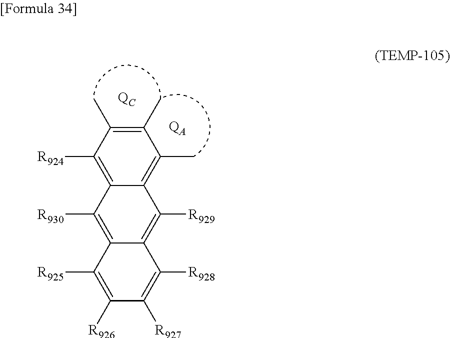

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TMEP-104) is a benzene ring, the ring Q_{A} is a monocyclic ring. When the ring Q_{A} in the formula (TMEP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, a substituent for the substituted or unsubstituted group (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms; R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when two or more R₉₀₁ are present, the two or more R₉₀₁ are mutually the same or different;

when two or more R₉₀₂ are present, the two or more R₉₀₂ are mutually the same or different;

when two or more R₉₀₃ are present, the two or more R₉₀₃ are mutually the same or different;

when two or more R₉₀₄ are present, the two or more R₉₀₄ are mutually the same or different;

when two or more R₉₀₅ are present, the two or more R₉₀₅ are mutually the same or different;

when two or more R₉₀₆ are present, the two or more R₉₀₆ are mutually the same or different; and

when two or more R₉₀₇ are present, the two or more R₉₀₇ are mutually the same or different.

In an exemplary embodiment, a substituent for the substituted or unsubstituted group is selected from the group consisting of an alkyl group having 1 to 50 carbon atoms, an aryl group having 6 to 50 ring carbon atoms, and a heterocyclic group having 5 to 50 ring atoms.

In an exemplary embodiment, a substituent for the substituted or unsubstituted group is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

### First Exemplary Embodiment

### Organic Electroluminescence Device

An organic electroluminescence device according to a first exemplary embodiment includes an anode, a cathode, a first emitting layer provided between the anode and the cathode, and a second emitting layer provided between the first emitting layer and the cathode. The first emitting layer contains, as a first host material, a first compound that has at least one group represented by a formula (11) below, the first compound being represented by a formula (1) below. The second emitting layer contains, as a second host material, a second compound represented by a formula (2) below. In the organic EL device according to the exemplary embodiment, the first emitting layer is in direct contact with the second emitting layer.

The organic electroluminescence device according to the exemplary embodiment includes the anode, the first emitting layer, the second emitting layer, and the cathode in this order.

Herein, a layer arrangement in which the first emitting layer is in direct contact with the second emitting layer can include one of embodiments (LS1), (LS2) and (LS3) below.

(LS1) An embodiment in which a region containing both the first compound and the second compound is generated in a process of vapor-depositing the compound of the first emitting layer and vapor-depositing the compound of the second emitting layer, and is present on the interface between the first emitting layer and the second emitting layer.

(LS2) An embodiment in which in a case of containing an emitting compound in the first emitting layer and the second emitting layer, a region containing all of the first compound, the second compound and the emitting compound is generated in a process of vapor-depositing the compound of the first emitting layer and vapor-depositing the compound of the second emitting layer, and is present on the interface between the first emitting layer and the second emitting layer.

(LS3) An embodiment in which in a case of containing an emitting compound in the first emitting layer and the second emitting layer, a region containing the emitting compound, a region containing the first compound or a region containing the second compound is generated in a process of vapor-depositing the compound of the first emitting layer and vapor-depositing the compound of the second emitting layer, and is present on the interface between the first emitting layer and the second emitting layer.

Herein, the “host material” refers to, for instance, a material that accounts for “50 mass % or more of the layer.” Accordingly, for instance, the first emitting layer contains 50 mass % or more of the first compound represented by the formula (1) below with respect to a total mass of the first emitting layer. The second emitting layer contains 50 mass % or more of the second compound represented by the formula (2) below with respect to a total mass of the second emitting layer.

### Emission Wavelength of Organic EL Device

The organic electroluminescence device according to the exemplary embodiment preferably emits light having a main peak wavelength in a range from 430 nm to 480 nm when the organic electroluminescence device is driven.

The main peak wavelength of the light emitted from the organic EL device when being driven is measured as follows. Voltage is applied on the organic EL devices such that a current density becomes 10 mA/cm², where spectral radiance spectrum is measured by a spectroradiometer CS-2000 (manufactured by Konica Minolta, Inc.). A peak wavelength of an emission spectrum, at which the luminous intensity of the resultant spectral radiance spectrum is at the maximum, is measured and defined as the main peak wavelength (unit: nm).

The organic EL device according to the exemplary embodiment may include one or more organic layer(s) in addition to the first emitting layer and the second emitting layer. Examples of the organic layer include, for instance, at least one layer selected from the group consisting of a hole injecting layer, a hole transporting layer, an emitting layer, an electron injecting layer, an electron transporting layer, a hole blocking layer, and an electron blocking layer.

In the organic EL device according to the exemplary embodiment, the organic layer may consist of the first emitting layer and the second emitting layer. Alternatively, the organic layer may further include, for instance, at least one layer selected from the group consisting of the hole injecting layer, the hole transporting layer, the electron injecting layer, the electron transporting layer, the hole blocking layer, and the electron blocking layer.

### Hole Transporting Layer

The organic EL device according to the exemplary embodiment preferably includes a hole transporting layer between the anode and the first emitting layer.

The organic EL device according to the exemplary embodiment preferably includes an electron transporting layer between the cathode and the second emitting layer.

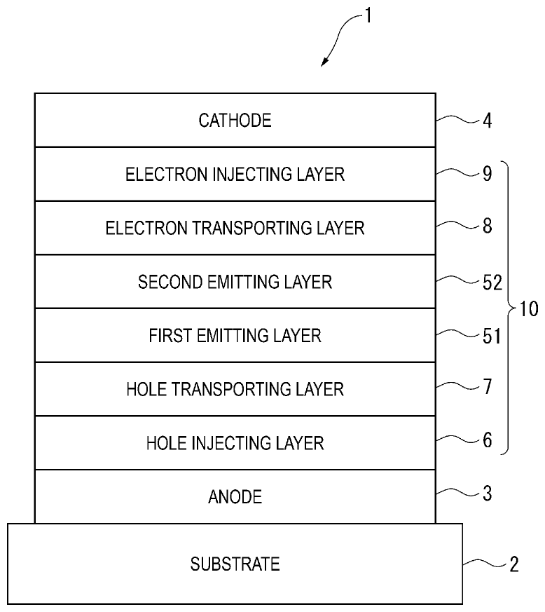

FIG. 1 schematically shows an exemplary arrangement of the organic EL device of the first exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, a first emitting layer 51, a second emitting layer 52, an electron transporting layer 8, and an electron injecting layer 9, which are sequentially laminated on the anode 3.

### First Compound

In the organic EL device according to the exemplary embodiment, the first compound is a compound represented by a formula (1) below.

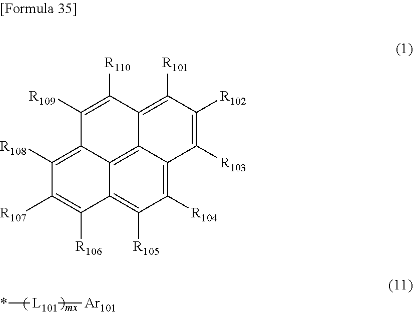

In the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a single bond, a substituted or unsubstituted arylene group having 6 to 50 ring carbon atoms, or a substituted or unsubstituted divalent heterocyclic group having 5 to 50 ring atoms;

Ar₁₀₁ is a substituted or unsubstituted aryl group having 6 to 50 ring carbon atoms, or a substituted or unsubstituted heterocyclic group having 5 to 50 ring atoms;

mx is 0, 1, 2, 3, 4, or 5;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

In the first compound of the exemplary embodiment, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₈₀₁ are present, the plurality of R₈₀₁ are mutually the same or different; and

when a plurality of R₈₀₂ are present, the plurality of R₈₀₂ are mutually the same or different.

In the organic EL device according to the exemplary embodiment, it is also preferable that in the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a single bond, or a substituted or unsubstituted arylene group having 6 to 50 ring carbon atoms;

Ar₁₀₁ is a substituted or unsubstituted aryl group having 6 to 50 ring carbon atoms;

mx is 0, 1, 2, 3, 4, or 5;

Ar₁₀₁ is not a substituted or unsubstituted pyrenyl group;

L₁₀₁ is not a substituted or unsubstituted pyrenylene group; a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different; and

when two or more Ar₁₀₁ are present, the two or more Ar₁₀₁ are mutually the same or different.

In the organic EL device according to the exemplary embodiment, the group represented by the formula (11) is preferably a group represented by a formula (111) below.

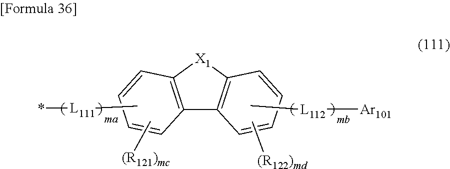

In the formula (111):

X₁ is CR₁₂₃R₁₂₄, an oxygen atom, a sulfur atom, or NR₁₂₅;

L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;

ma is 0, 1, 2, 3, or 4;

mb is 0, 1, 2, 3, or 4;

ma+mb is 0, 1, 2, 3, or 4;

Ar₁₀₁ represents the same as Ar₁₀₁ in the formula (11);

R₁₂₁, R₁₂₂, R₁₂₃, R₁₂₄ and R₁₂₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

mc is 3;

three R₁₂₁ are mutually the same or different;

md is 3; and

three R₁₂₂ are mutually the same or different.

In the organic EL device according to the exemplary embodiment, it is preferable that in the formula (111):

X₁ is CR₁₂₃R₁₂₄,

L₁₁₁ and L₁₁₂ are each independently a single bond, or a substituted or unsubstituted arylene group having 6 to 50 ring carbon atoms;

ma is 0, 1, 2, 3, or 4;

mb is 0, 1, 2, 3, or 4;

ma+mb is 0, 1, 2, 3, or 4;

Ar₁₀₁ represents the same as Ar₁₀₁ in the formula (11);

R₁₂₁, R₁₂₂, R₁₂₃ and R₁₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, or a substituted or unsubstituted aryl group having 6 to 50 ring carbon atoms;

mc is 3;

three R₁₂₁ are mutually the same or different;

md is 3; and

three R₁₂₂ are mutually the same or different.

Among positions *1 to *8 of carbon atoms in a cyclic structure represented by a formula (111a) below in a group represented by the formula (111), L₁₁₁ is bonded to one of the positions *1 to *4, R₁₂₁ is bonded to each of three positions of the rest of *1 to *4, L₁₁₂ is bonded to one of the positions *5 to *8, and R₁₂₂ is bonded to each of three positions of the rest of *5 to *8.

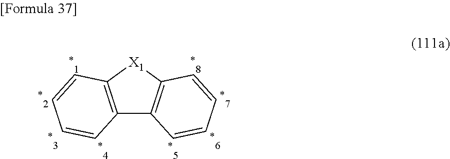

For instance, in a group represented by the formula (111), when L₁₁₁ is bonded to a carbon atom at the position *2 in the cyclic structure represented by the formula (111a) and L₁₁₂ is bonded to a carbon atom at the position *7 in the cyclic structure represented by the formula (111a), the group represented by the formula (111) is represented by a formula (111b) below.

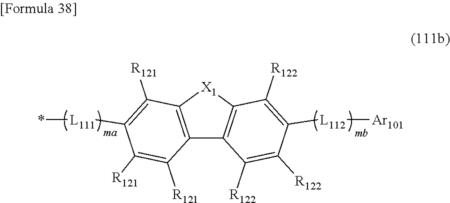

In the formula (111b):

X₁, L₁₁₁, L₁₁₂, ma, mb, Ar₁₀₁, R₁₂₁, R₁₂₂, R₁₂₃, R₁₂₄ and R₁₂₅ each independently represent the same as X₁, L₁₁₁, L₁₁₂, ma, mb, Ar₁₀₁, R₁₂₁, R₁₂₂, R₁₂₃, R₁₂₄ and R₁₂₅ in the formula (111);

a plurality of R₁₂₁ are mutually the same or different; and

a plurality of R₁₂₂ are mutually the same or different.

In the organic EL device according to the exemplary embodiment, the group represented by the formula (111) is preferably a group represented by the formula (111b).

In the organic EL device according to the exemplary embodiment, it is preferable that ma is 0, 1, or 2; and mb is 0, 1, or 2.

In the organic EL device according to the exemplary embodiment, it is preferable that ma is 0 or 1; and mb is 0 or 1.

In the organic EL device according to the exemplary embodiment, Ar₁₀₁ is preferably a substituted or unsubstituted aryl group having 6 to 50 ring carbon atoms.

In the organic EL device according to the exemplary embodiment, it is preferable that Ar₁₀₁ is a substituted or unsubstituted phenyl group, a substituted or unsubstituted naphthyl group, a substituted or unsubstituted biphenyl group, a substituted or unsubstituted terphenyl group, a substituted or unsubstituted pyrenyl group, a substituted or unsubstituted phenanthryl group, or a substituted or unsubstituted fluorenyl group.

In the organic EL device according to the exemplary embodiment, Ar₁₀₁ is also preferably a group represented by a formula (12), a formula (13), or a formula (14) below.

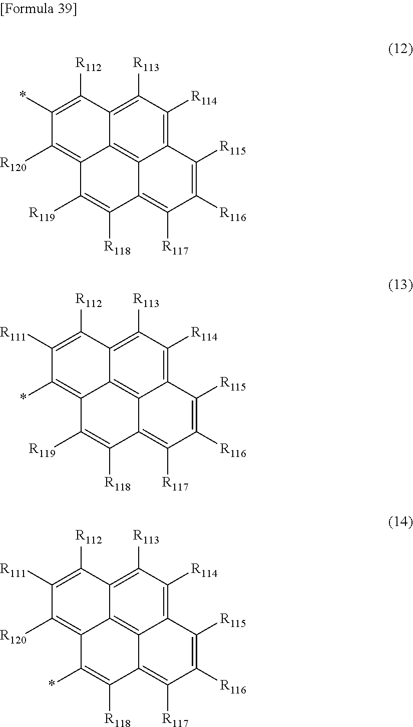

In the formulae (12), (13) and (14):

R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₁₂₄, a group represented by —COOR₁₂₅, a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formulae (12), (13) and (14) represents a bonding position to L₁₀₁ in the formula (11), or a bonding position to L₁₁₂ in the formula (111) or (111b).

In the organic EL device according to the exemplary embodiment, the first compound is preferably represented by a formula (101) below.

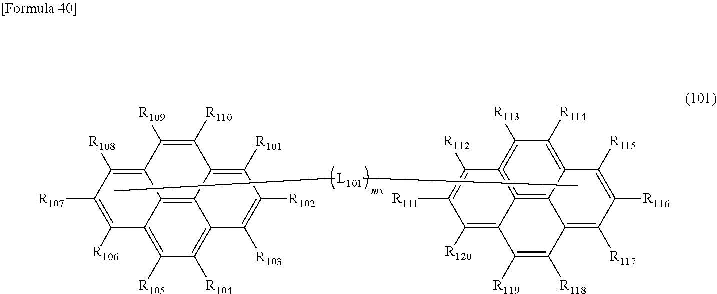

In the formula (101):

R₁₀₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

one of R₁₀₁ to R₁₁₀ represents a bonding position to L₁₀₁, and one of R₁₁₁ to R₁₂₀ represents a bonding position to L₁₀₁;

L₁₀₁ is a single bond, a substituted or unsubstituted arylene group having 6 to 50 ring carbon atoms, or a substituted or unsubstituted divalent heterocyclic group having 5 to 50 ring atoms;

mx is 0, 1, 2, 3, 4, or 5; and

when two or more of L₁₀₁ are present, the two or more of L₁₀₁ are mutually the same or different.

In the organic EL device according to the exemplary embodiment, it is preferable that L₁₀₁ is a single bond, or a substituted or unsubstituted arylene group having 6 to 50 ring carbon atoms.

In the organic EL device according to the exemplary embodiment, it is preferable that the first compound is represented by a formula (102) below.

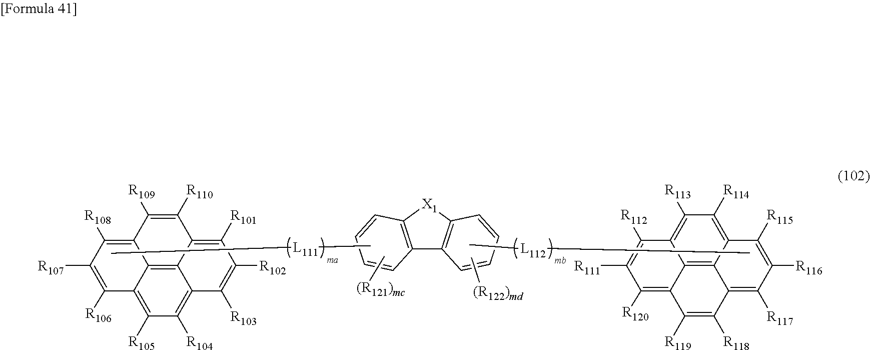

In the formula (102):

R₁₀₁ to R₁₂₀ each independently represent the same as R₁₀₁ to R₁₂₀ in the formula (101);

one of R₁₀₁ to R₁₁₀ represents a bonding position to L₁₁₁, and one of R₁₁₁ to R₁₂₀ represents a bonding position to L₁₁₂;

X₁ is CR₁₂₃R₁₂₄, an oxygen atom, a sulfur atom, or NR₁₂₅;

L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;

ma is 0, 1, 2, 3, or 4;

mb is 0, 1, 2, 3, or 4;

ma+mb is 0, 1, 2, 3, or 4;

R₁₂₁, R₁₂₂, R₁₂₃, R₁₂₄ and R₁₂₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

mc is 3;

three R₁₂₁ are mutually the same or different;

md is 3; and

three R₁₂₂ are mutually the same or different.

In the compound represented by the formula (102), it is preferable that ma is 0, 1, or 2; and mb is 0, 1, or 2.

In the compound represented by the formula (102), it is preferable that ma is 0 or 1; and mb is 0 or 1.

In the organic EL device according to the exemplary embodiment, it is preferable that two or more of R₁₀₁ to R₁₁₀ are each a group represented by the formula (11).

In the organic EL device according to the exemplary embodiment, it is preferable that two or more of R₁₀₁ to R₁₁₀ are each a group represented by the formula (11) and Ar₁₀₁ is a substituted or unsubstituted aryl group having 6 to 50 ring carbon atoms.

In the organic EL device according to the exemplary embodiment, it is preferable that Ar₁₀₁ is not a substituted or unsubstituted pyrenyl group; L₁₀₁ is not a substituted or unsubstituted pyrenylene group; and a substituted or unsubstituted aryl group having 6 to 50 ring carbon atoms as R₁₀₁ to R₁₁₀ not being each a group represented by the formula (11) is not a substituted or unsubstituted pyrenyl group.

In the organic EL device according to the exemplary embodiment, examples of the substituent for a “substituted or unsubstituted group” also preferably do not include a substituted or unsubstituted pyrenyl group.

When Ar₁₀₁ is a substituted aryl group having 6 to 50 ring carbon atoms, a substituent of the substituted aryl group as Ar₁₀₁ is also preferably not a heterocyclic group.

In the organic EL device according to the exemplary embodiment, it is also preferable that the first compound contains at least two groups represented by the formula (11).

When the first compound contains at least two groups represented by the formula (11), it is also preferable that in the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, or a group represented by the formula (11);

two or more of R₁₀₁ to R₁₁₀ are each a group represented by the formula (11);

when a plurality of groups represented by the formula (11) are present, the plurality of groups represented by the formula (11) are mutually the same or different;

L₁₀₁ is a single bond;

Ar₁₀₁ is a substituted or unsubstituted heterocyclic group having 5 to 50 ring atoms;

mx is 0; and

when two or more Ar₁₀₁ are present, the two or more Ar₁₀₁ are mutually the same or different.

When mx is 0, Ar₁₀₁ is bonded to a pyrene ring in the formula (1) with a single bond.

In the organic EL device according to the exemplary embodiment, two or three of R₁₀₁ to R₁₁₀ are preferably each a group represented by the formula (11).

In the organic EL device according to the exemplary embodiment, it is also preferable that in the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a substituted or unsubstituted divalent heterocyclic group having 5 to 50 ring atoms;

Ar₁₀₁ is a substituted or unsubstituted heterocyclic group having 5 to 50 ring atoms;

mx is 1, 2, 3, 4 or 5;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different; and

when two or more Ar₁₀₁ are present, the two or more Ar₁₀₁ are mutually the same or different.

In the organic EL device according to the exemplary embodiment, it is also preferable that in the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

at least one of R₁₀₁ to R₁₁₀ is a substituted or unsubstituted heterocyclic group having 5 to 50 ring atoms;

when a plurality of groups represented by the formula (11) are present, the plurality of groups represented by the formula (11) are mutually the same or different;

L₁₀₁ is a single bond, or a substituted or unsubstituted arylene group having 6 to 50 ring carbon atoms;

Ar₁₀₁ is a substituted or unsubstituted aryl group having 6 to 50 ring carbon atoms;

mx is 0, 1, 2, 3, 4, or 5;

Ar₁₀₁ is not a substituted or unsubstituted pyrenyl group;

L₁₀₁ is not a substituted or unsubstituted pyrenylene group;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different; and

when two or more Ar₁₀₁ are present, the two or more Ar₁₀₁ are mutually the same or different.

A substituted or unsubstituted heterocyclic group having 5 to 50 ring atoms as R₁₀₁ to R₁₁₀ is also preferably a heterocyclic group selected from the group consisting of a monocyclic heterocyclic group, a heterocyclic group in which two rings are fused, and a heterocyclic group in which three rings are fused.

In the organic EL device according to the exemplary embodiment, one or two of R₁₀₁ to R₁₁₀ are also preferably each a group represented by the formula (11).

In the organic EL device according to the exemplary embodiment, it is also preferable that mx is 1, 2, 3, 4, or 5 and L₁₀₁ is a substituted or unsubstituted arylene group having 6 to 50 ring carbon atoms.

In the organic EL device according to the exemplary embodiment, it is preferable that in the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a substituted or unsubstituted arylene group having 6 to 50 ring carbon atoms, or a substituted or unsubstituted divalent heterocyclic group having 5 to 50 ring atoms;

Ar₁₀₁ is a substituted or unsubstituted heterocyclic group having 5 to 50 ring atoms;

mx is 1, 2, 3, 4 or 5;

Ar₁₀₁ is a substituted or unsubstituted heterocyclic group having 5 to 50 ring atoms;

mx is 1, 2, 3, 4 or 5;

L₁₀₁ is not a substituted or unsubstituted pyrenylene group;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different; and

when two or more of Ar₁₀₁ are present, the two or more of Ar₁₀₁ are mutually the same or different. L₁₀₁ is more preferably a substituted or unsubstituted arylene group having 6 to 50 ring carbon atoms.

In the organic EL device according to the exemplary embodiment, it is also preferable that in the formula (1):

R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);

at least one of R₁₀₁ to R₁₁₀ is a group represented by the formula (11);

when a plurality of groups represented by the formula (11) are present, the plurality of groups represented by the formula (11) are mutually the same or different;

L₁₀₁ is a substituted or unsubstituted divalent heterocyclic group having 5 to 50 ring atoms;

Ar₁₀₁ is a substituted or unsubstituted aryl group having 6 to 50 ring carbon atoms;

mx is 1, 2, 3, 4 or 5;

Ar₁₀₁ is not a substituted or unsubstituted pyrenyl group;

a substituted or unsubstituted aryl group having 6 to 50 ring carbon atoms as R₁₀₁ to R₁₁₀ not being a group represented by the formula (11) is not a substituted or unsubstituted pyrenyl group;

when two or more L₁₀₁ are present, the two or more L₁₀₁ are mutually the same or different;

when two or more Ar₁₀₁ are present, the two or more Ar₁₀₁ are mutually the same or different; and

* in the formula (11) represents a bonding position to a pyrene ring in the formula (1).

In the organic EL device according to the exemplary embodiment, it is also preferable that L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group, or at least one divalent heterocyclic group selected from the group consisting of a substituted or unsubstituted dibenzofurandiyl group, a substituted or unsubstituted dibenzothiophenediyl group, and a substituted or unsubstituted carbazolediyl group.

In the organic EL device according to the exemplary embodiment, it is also preferable that at least one L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group.

In the organic EL device according to the exemplary embodiment, it is also preferable that at least one L₁₀₁ is at least one divalent heterocyclic group selected from the group consisting of a substituted or unsubstituted dibenzofurandiyl group, a substituted or unsubstituted dibenzothiophenediyl group, and a substituted or unsubstituted carbazolediyl group.

In the organic EL device according to the exemplary embodiment, it is also preferable that mx is 1, 2, 3, 4, or 5, and at least one L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group.

In the organic EL device according to the exemplary embodiment, it is also preferable that mx is 1, 2, 3, 4, or 5, and at least one L₁₀₁ is at least one divalent heterocyclic group selected from the group consisting of a substituted or unsubstituted dibenzofurandiyl group, a substituted or unsubstituted dibenzothiophenediyl group, and a substituted or unsubstituted carbazolediyl group.

In the organic EL device according to the exemplary embodiment, Ar₁₀₁ is also preferably an aryl group in which four or more rings are fused.

In the organic EL device according to the exemplary embodiment, Ar₁₀₁ is also preferably a heterocyclic group in which four or more rings are fused.

In the organic EL device according to the exemplary embodiment, it is also preferable that Ar₁₀₁ is a heterocyclic group in which four or more rings are fused, mx is 1, 2, 3, 4, or 5, and at least one L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group.

In an exemplary embodiment, the first compound is a compound having only one pyrene ring in a molecule (sometimes referred to as a monopyrene compound).

When the first compound is a monopyrene compound and Ar₁₀₁ is an aryl group in which four or more rings are fused or a heterocyclic group in which four or more rings are fused, it is preferable that mx is 1, 2, 3, 4, or 5 and at least one L₁₀₁ is not a single bond (i.e., L₁₀₁ is an arylene group or a divalent heterocyclic group) and it is more preferable that at least one L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group, or at least one divalent heterocyclic group selected from the group consisting of a substituted or unsubstituted dibenzofurandiyl group, a substituted or unsubstituted dibenzothiophenediyl group, and a substituted or unsubstituted carbazolediyl group. Presence of such a linking group between a pyrene ring and an aryl group in which four or more rings are fused or a heterocyclic group in which four or more rings are fused in a monopyrene compound can suppress a decrease in hole mobility and also suppress a decrease in luminous efficiency of the organic EL device. Since the linking group is likely to decrease electron injectability, the linking group preferably does not contain a carbazole ring. In a monopyrene compound, presence of a linking group between a pyrene ring and an aryl group in which four or more rings are fused or a heterocyclic group in which four or more rings are fused can suppress a decrease in electron injectability into an emitting layer containing the monopyrene compound and also suppress a decrease in luminous efficiency of the organic EL device.

In the organic EL device according to the exemplary embodiment, Ar₁₀₁ is also preferably a substituted or unsubstituted phenyl group, a substituted or unsubstituted naphthyl group, a substituted or unsubstituted biphenyl group, a substituted or unsubstituted terphenyl group, a substituted or unsubstituted phenanthryl group, or a substituted or unsubstituted fluorenyl group.

In the organic EL device according to the exemplary embodiment, one of R₁₀₁ to R₁₁₀ is also preferably a group represented by the formula (11).

In the organic EL device according to the exemplary embodiment, mx is also preferably 1.

In the organic EL device according to the exemplary embodiment, it is preferable that R₁₀₁ to R₁₁₀ not being each a group represented by the formula (11) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that R₁₀₁ to R₁₁₀ not being each a group represented by the formula (11) are each independently a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₀₁ to R₁₁₀ not being each a group represented by the formula (11) are each preferably a hydrogen atom.

In the first compound and the second compound, it is preferable that all groups defined to be substituted or unsubstituted in the first compound and the second compound are unsubstituted groups.

In the organic EL device according to the exemplary embodiment, for instance, two of R₁₀₁ to R₁₁₀ in the first compound represented by the formula (1) are groups represented by the formula (11).

In the organic EL device according to the exemplary embodiment, for instance, three of R₁₀₁ to R₁₁₀ in the first compound represented by the formula (1) are groups represented by the formula (11).

In the organic EL device according to the exemplary embodiment, for instance, four of R₁₀₁ to R₁₁₀ in the first compound represented by the formula (1) are groups represented by the formula (11).

In the organic EL device according to the exemplary embodiment, for instance, one of R₁₀₁ to R₁₁₀ in the first compound represented by the formula (1) is a group represented by the formula (11) and mx is 1 or more.

In the organic EL device according to the exemplary embodiment, for instance, one of R₁₀₁ to R₁₁₀ in the first compound represented by the formula (1) is a group represented by the formula (11), mx is 0, and Ar₁₀₁ is a substituted or unsubstituted aryl group.

In the organic EL device according to the exemplary embodiment, for instance, one of R₁₀₁ to R₁₁₀ in the first compound represented by the formula (1) is a group represented by the formula (11), mx is 0, and Ar₁₀₁ is a substituted or unsubstituted heterocyclic group containing a nitrogen atom.

In the organic EL device according to the exemplary embodiment, for instance, one of R₁₀₁ to R₁₁₀ in the first compound represented by the formula (1) is a group represented by the formula (11), mx is 0, and Ar₁₀₁ is a substituted or unsubstituted heterocyclic group containing a sulfur atom.

In the organic EL device according to the exemplary embodiment, for instance, one of R₁₀₁ to R₁₁₀ in the first compound represented by the formula (1) is a group represented by the formula (11), mx is 0, and Ar₁₀₁ is a substituted or unsubstituted furyl group, oxazolyl group, isoxazolyl group, oxadiazolyl group, xanthenyl group, benzofuranyl group, isobenzofuranyl group, dibenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

In the organic EL device according to the exemplary embodiment, for instance, one of R₁₀₁ to R₁₁₀ in the first compound represented by the formula (1) is a group represented by the formula (11), mx is 0, and Ar₁₀₁ is at least one group selected from the group consisting of unsubstituted furyl group, oxazolyl group, isoxazolyl group, oxadiazolyl group, xanthenyl group, benzofuranyl group, isobenzofuranyl group, dibenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

In the organic EL device according to the exemplary embodiment, for instance, one of R₁₀₁ to R₁₁₀ in the first compound represented by the formula (1) is a group represented by the formula (11), mx is 0, and Ar₁₀₁ is a substituted or unsubstituted dibenzofuranyl group.

In the organic EL device according to the exemplary embodiment, for instance, one of R₁₀₁ to R₁₁₀ in the first compound represented by the formula (1) is a group represented by the formula (11), mx is 0, and Ar₁₀₁ is an unsubstituted dibenzofuranyl group.

In the organic EL device according to the exemplary embodiment, for instance, mx in the first compound represented by the formula (101) is 2 or more.

In the organic EL device according to the exemplary embodiment, for instance, mx in the first compound represented by the formula (101) is 1 or more, and L₁₀₁ is an arylene group having 6 to 24 ring carbon atoms or a divalent heterocyclic group having 5 to 24 ring atoms.

In the organic EL device according to the exemplary embodiment, for instance, mx in the first compound represented by the formula (101) is 1 or more, and L₁₀₁ is an arylene group having 6 to 18 ring carbon atoms or a divalent heterocyclic group having 5 to 18 ring atoms.

In the organic EL device according to the exemplary embodiment, it is also preferable that the first compound contains at least one group represented by a formula (11A) below.

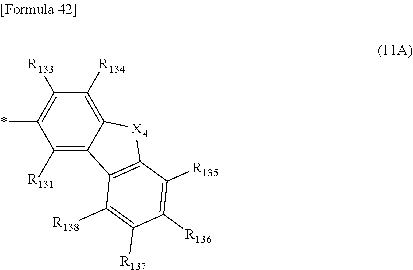

In the formula (11A):

X_{A} is an oxygen atom or a sulfur atom;

at least one combination of adjacent two or more of R₁₃₁, R₁₃₃ and R₁₃₄ are mutually bonded to form a substituted or unsubstituted monocyclic ring, mutually bonded to form a substituted or unsubstituted fused ring, or not mutually bonded;

R₁₃₅ to R₁₃₈, and R₁₃₁, R₁₃₃ and R₁₃₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formula (11A) represents a bonding position to a pyrene ring in the formula (1).

When the first compound contains at least one group represented by the formula (11A), it is preferable that in the formula (1) R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11A);

at least one of R₁₀₁ to R₁₁₀ is a group represented by the formula (11A); and

when a plurality of groups represented by the formula (11A) are present, the plurality of groups represented by the formula (11A) are mutually the same or different.

R₁₃₅ to R₁₃₈ do not bonded to each other to form no substituted or unsubstituted monocyclic ring and no substituted or unsubstituted fused ring.

In the organic EL device according to the exemplary embodiment, it is also preferable that the first compound contains one group represented by the formula (11A).

In the organic EL device according to the exemplary embodiment, R₁₀₁, R₁₀₃, R₁₀₆ or R₁₀₈ is also preferably a group represented by the formula (11A).

In the organic EL device according to the exemplary embodiment, R₁₃₅ to R₁₃₈ are preferably a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, or a nitro group.

It is also preferable that R₁₃₅ to R₁₃₈, and R₁₃₁, R₁₃₃ and R₁₃₄ not forming the substituted or unsubstituted monocyclic ring and not forming the substituted or unsubstituted fused ring in a group represented by the formula (11A) are not each a substituted or unsubstituted pyrenyl group, and a substituted or unsubstituted aryl group having 6 to 50 ring carbon atoms as R₁₀₁ to R₁₁₀ not being a group represented by the formula (11A) is not a substituted or unsubstituted pyrenyl group.

In the organic EL device according to the exemplary embodiment, it is also preferable that the first compound contains at least one group represented by a formula (11B) below.

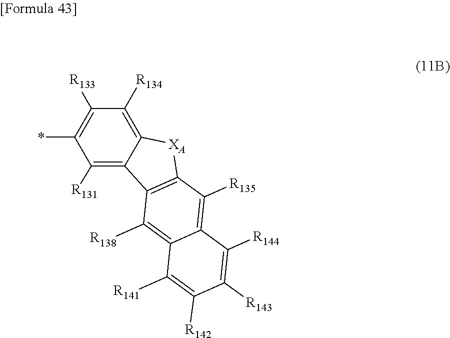

In the formula (11B):

X_{A} is an oxygen atom or a sulfur atom;

at least one combination of adjacent two or more of R₁₃₁, R₁₃₃ to R₁₃₅, R₁₃₈ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃ to R₁₃₅, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

at least one of R₁₃₅, R₁₃₈ or R₁₄₁ to R₁₄₄ are a substituted or unsubstituted aryl group having 6 to 50 ring carbon atoms, or a substituted or unsubstituted heterocyclic group having 5 to 50 ring atoms; and

* in the formula (11B) represents a bonding position to a pyrene ring in the formula (1).

When the first compound contains at least one group represented by the formula (11B), it is preferable that in the formula (1) R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11B);

at least one of R₁₀₁ to R₁₁₀ is a group represented by the formula (11B); and

when a plurality of groups represented by the formula (11B) are present, the plurality of groups represented by the formula (11B) are mutually the same or different.

In the organic EL device according to the exemplary embodiment, it is also preferable that the first compound contains one group represented by the formula (11B).

In the organic EL device according to the exemplary embodiment, R₁₀₁, R₁₀₃, R₁₀₆ or R₁₀₈ is also preferably a group represented by the formula (11B).

It is preferable that R₁₃₁, R₁₃₃ to R₁₃₅, R₁₃₈, and R₁₄₁ to R₁₄₄ not forming the substituted or unsubstituted monocyclic ring and not forming the substituted or unsubstituted fused ring in a group represented by the formula (11B) are not each a substituted or unsubstituted pyrenyl group, and a substituted or unsubstituted aryl group having 6 to 50 ring carbon atoms as R₁₀₁ to R₁₁₀ not being a group represented by the formula (11B) is not a substituted or unsubstituted pyrenyl group.

In the organic EL device according to the exemplary embodiment, it is also preferable that the first compound contains at least one group represented by a formula (11C) below.

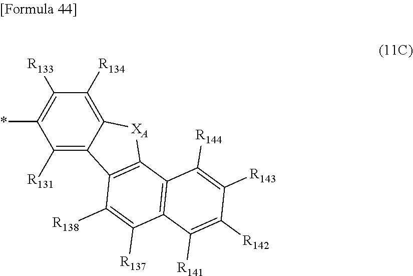

In the formula (11C):

X_{A} is an oxygen atom or a sulfur atom;

at least one combination of adjacent two or more of R₁₃₁, R₁₃₃, R₁₃₄, R₁₃₇, R₁₃₈ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃, R₁₃₄, R₁₃₇, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formula (11C) represents a bonding position to a pyrene ring in the formula (1).

When the first compound contains at least one group represented by the formula (11C), it is preferable that in the formula (1) R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11C);

at least one of R₁₀₁ to R₁₁₀ is a group represented by the formula (11C); and

when a plurality of groups represented by the formula (11C) are present, the plurality of groups represented by the formula (11C) are mutually the same or different.

In the organic EL device according to the exemplary embodiment, it is also preferable that the first compound contains one group represented by the formula (11C).

In the organic EL device according to the exemplary embodiment, R₁₀₁, R₁₀₃, R₁₀₆ or R₁₀₈ is also preferably a group represented by the formula (11C).

It is preferable that R₁₃₁, R₁₃₃, R₁₃₄, R₁₃₇, R₁₃₈, and R₁₄₁ to R₁₄₄ not forming the substituted or unsubstituted monocyclic ring and not forming the substituted or unsubstituted fused ring in a group represented by the formula (11C) are not each a substituted or unsubstituted pyrenyl group, and a substituted or unsubstituted aryl group having 6 to 50 ring carbon atoms as R₁₀₁ to R₁₁₀ each not being a group represented by the formula (11C) is not a substituted or unsubstituted pyrenyl group.

In the organic EL device according to the exemplary embodiment, it is also preferable that the first compound contains at least one group represented by a formula (11D) below.

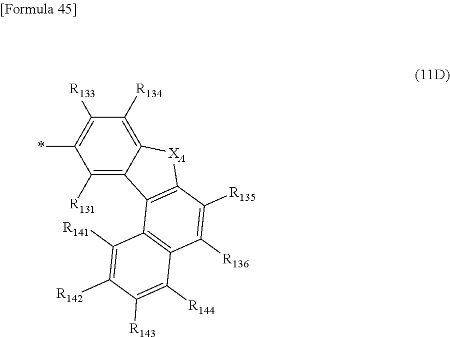

In the formula (11D):

X_{A} is an oxygen atom or a sulfur atom;

at least one combination of adjacent two or more of R₁₃₁, R₁₃₃, R₁₃₄, R₁₃₅, R₁₃₆ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃, R₁₃₄, R₁₃₅, R₁₃₆ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formula (11D) represents a bonding position to a pyrene ring in the formula (1).

When the first compound contains at least one group represented by the formula (11D), it is preferable that in the formula (1) R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11D);

at least one of R₁₀₁ to R₁₁₀ is a group represented by the formula (11D); and

when a plurality of groups represented by the formula (11D) are present, the plurality of groups represented by the formula (11D) are mutually the same or different.

In the organic EL device according to the exemplary embodiment, it is also preferable that the first compound contains one group represented by the formula (11D).

In the organic EL device according to the exemplary embodiment, R₁₀₁, R₁₀₃, R₁₀₆ or R₁₀₈ is also preferably a group represented by the formula (11D).

It is preferable that R₁₃₁, R₁₃₃, R₁₃₄, R₁₃₅, R₁₃₆, and R₁₄₁ to R₁₄₄ not forming the substituted or unsubstituted monocyclic ring and not forming the substituted or unsubstituted fused ring in a group represented by the formula (11D) are not each a substituted or unsubstituted pyrenyl group, and a substituted or unsubstituted aryl group having 6 to 50 ring carbon atoms as R₁₀₁ to R₁₁₀ each not being a group represented by the formula (11D) is not a substituted or unsubstituted pyrenyl group.

In the organic EL device according to the exemplary embodiment, it is also preferable that the first compound contains at least one group represented by a formula (11E) below.

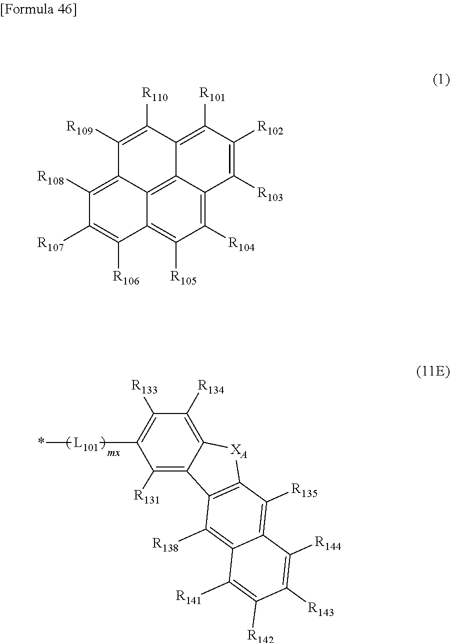

In the formula (11E):

X_{A} is an oxygen atom or a sulfur atom;

at least one combination of adjacent two or more of R₁₃₁, R₁₃₃ to R₁₃₅, R₁₃₈ and R₁₄₁ to R₁₄₄ are mutually bonded to form a substituted or unsubstituted monocyclic ring, mutually bonded to form a substituted or unsubstituted fused ring, or not mutually bonded;

R₁₃₁, R₁₃₃ to R₁₃₅, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms;

L₁₀₁ is a substituted or unsubstituted arylene group having 6 to 50 ring carbon atoms, or a substituted or unsubstituted divalent heterocyclic group having 5 to 50 ring atoms;

mx is 1, 2, 3, 4 or 5;

when two or more L₁₀₁ are present, the two or more L₁₀₁ are mutually the same or different; and

* in the formula (11E) represents a bonding position to a pyrene ring in the formula (1).

When the first compound contains at least one group represented by the formula (11E), it is preferable that in the formula (1) R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11E);

at least one of R₁₀₁ to R₁₁₀ is a group represented by the formula (11E); and

when a plurality of groups represented by the formula (11E) are present, the plurality of groups represented by the formula (11E) are mutually the same or different.

In the organic EL device according to the exemplary embodiment, it is also preferable that the first compound contains one group represented by the formula (11E).

In the organic EL device according to the exemplary embodiment, R₁₀₁, R₁₀₃, R₁₀₆ or R₁₀₈ is also preferably a group represented by the formula (11E).

It is preferable that R₁₃₁, R₁₃₃ to R₁₃₅, R₁₃₈, and R₁₄₁ to R₁₄₄ not forming the substituted or unsubstituted monocyclic ring and not forming the substituted or unsubstituted fused ring in a group represented by the formula (11E) are not each a substituted or unsubstituted pyrenyl group, and a substituted or unsubstituted aryl group having 6 to 50 ring carbon atoms as R₁₀₁ to R₁₁₀ each not being a group represented by the formula (11E) is not a substituted or unsubstituted pyrenyl group.

In the organic EL device according to the exemplary embodiment, a group represented by the formula (11) is also preferably a group represented by the formula (111E).

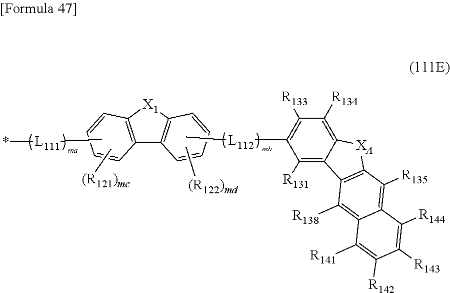

In the formula (111E):

X₁ is CR₁₂₃R₁₂₄, an oxygen atom, a sulfur atom, or NR₁₂₅;

L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;

ma is 1, 2, 3, or 4;

mb is 1, 2, 3, or 4;

ma+mb is 2, 3, or 4;

X_{A}, R₁₃₁, R₁₃₃ to R₁₃₅, R₁₃₈ and R₁₄₁ to R₁₄₄ each represent the same as X_{A}, R₁₃₁, R₁₃₃ to R₁₃₅, R₁₃₈ and R₁₄₁ to R₁₄₄ in the formula (11E);

R₁₂₁, R₁₂₂, R₁₂₃, R₁₂₄ and R₁₂₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

mc is 3;

three R₁₂₁ are mutually the same or different;

md is 3; and

three R₁₂₂ are mutually the same or different.

In the organic EL device according to the exemplary embodiment, it is preferable that ma is 1 or 2 and mb is 1 or 2.

In the organic EL device according to the exemplary embodiment, it is preferable that ma is 1 and mb is 1.

In the organic EL device according to the exemplary embodiment, R₁₀₁ to R₁₁₀ not being a group represented by the formula (11A), the formula (11B), the formula (11C), the formula (11D), the formula (11E) or the formula (111E) are each independently preferably a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the exemplary embodiment, R₁₀₁ to R₁₁₀ not being a group represented by the formula (11A), the formula (11B), the formula (11C), the formula (11D), the formula (11E) or the formula (111E) are each independently preferably a hydrogen atom, a substituted or unsubstituted alkyl group having 1 to 50 carbon atoms, or a substituted or unsubstituted cycloalkyl group having 3 to 50 ring carbon atoms.

In the organic EL device according to the exemplary embodiment, R₁₀₁ to R₁₁₀ not being a group represented by the formula (11A), the formula (11B), the formula (11C), the formula (11D), the formula (11E) or the formula (111E) are each preferably a hydrogen atom.

### Manufacturing Method of First Compound

The first compound can be manufactured by a known method. The first compound can also be manufactured based on a known method through a known alternative reaction using a known material(s) tailored for the target compound.

### Specific Examples of First Compound

Specific examples of the first compound include the following compounds. It should however be noted that the invention is not limited to the specific examples of the first compound.

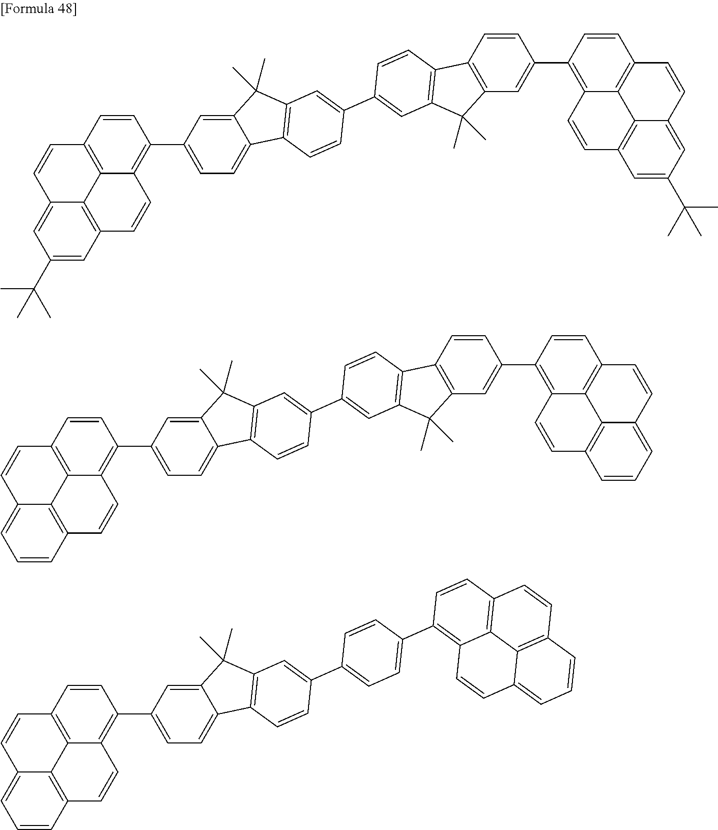

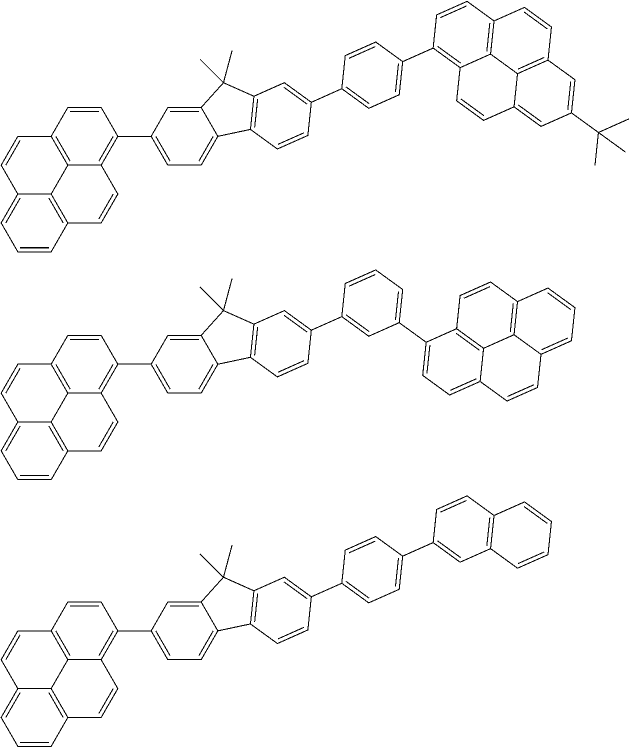

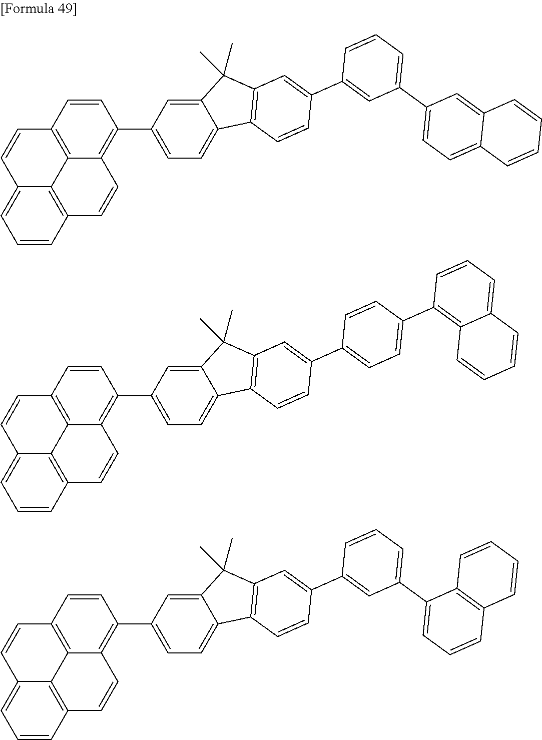

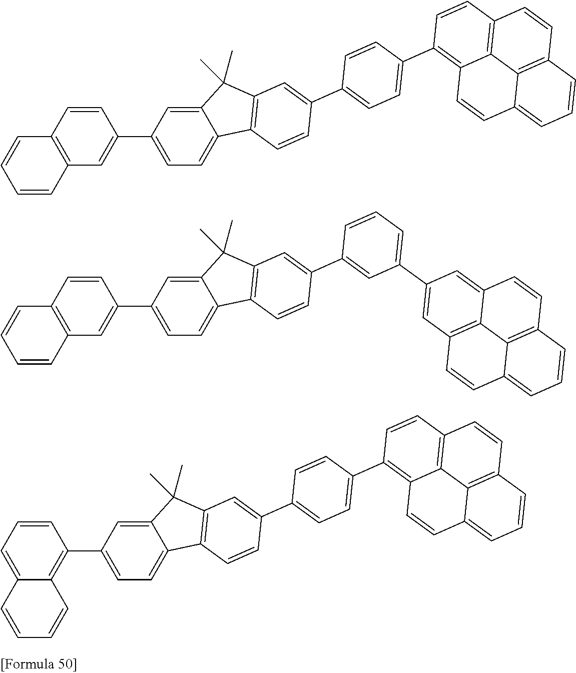

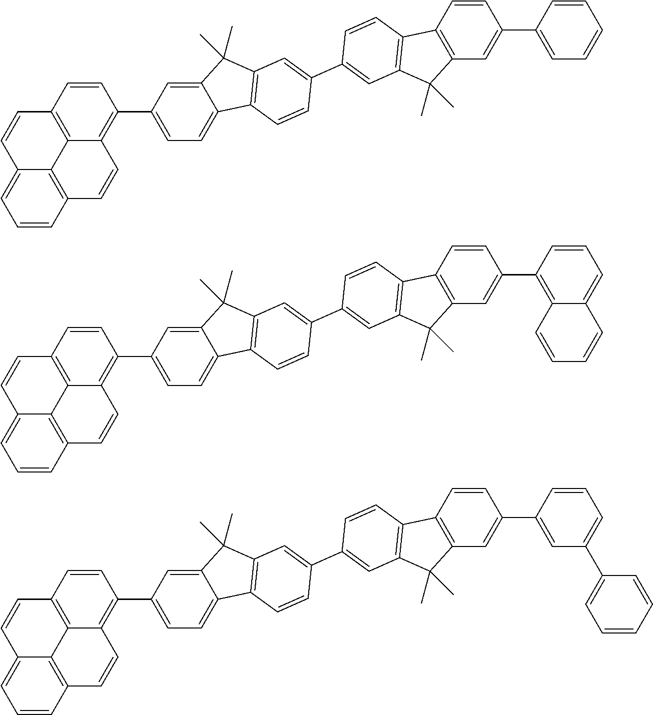

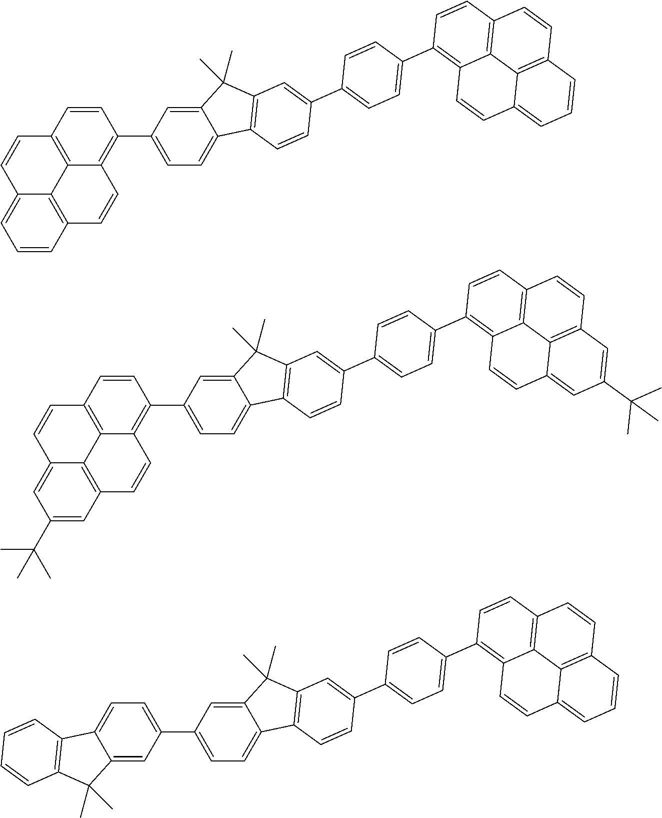

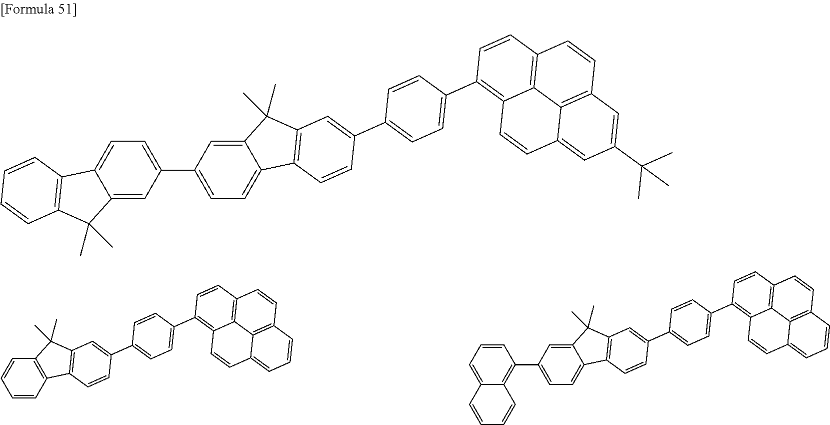

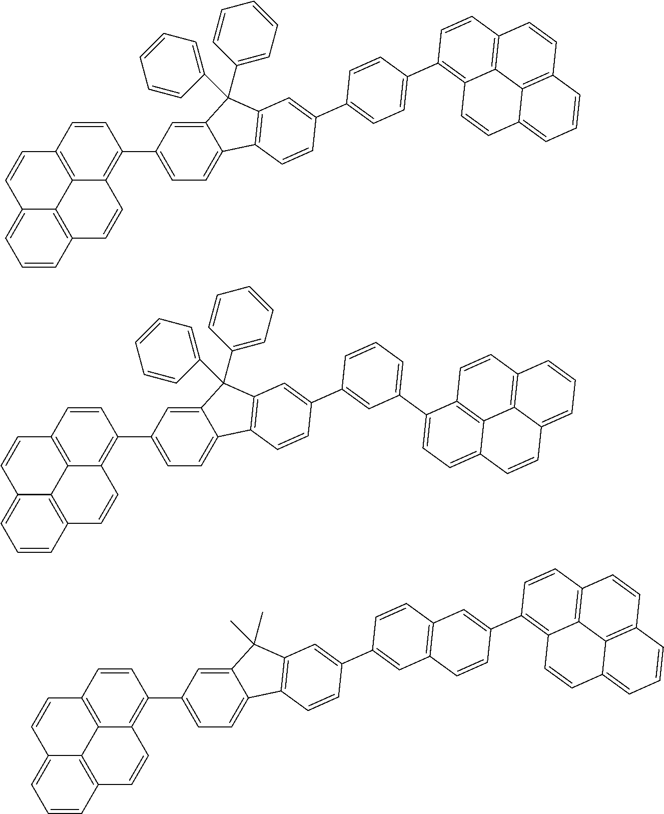

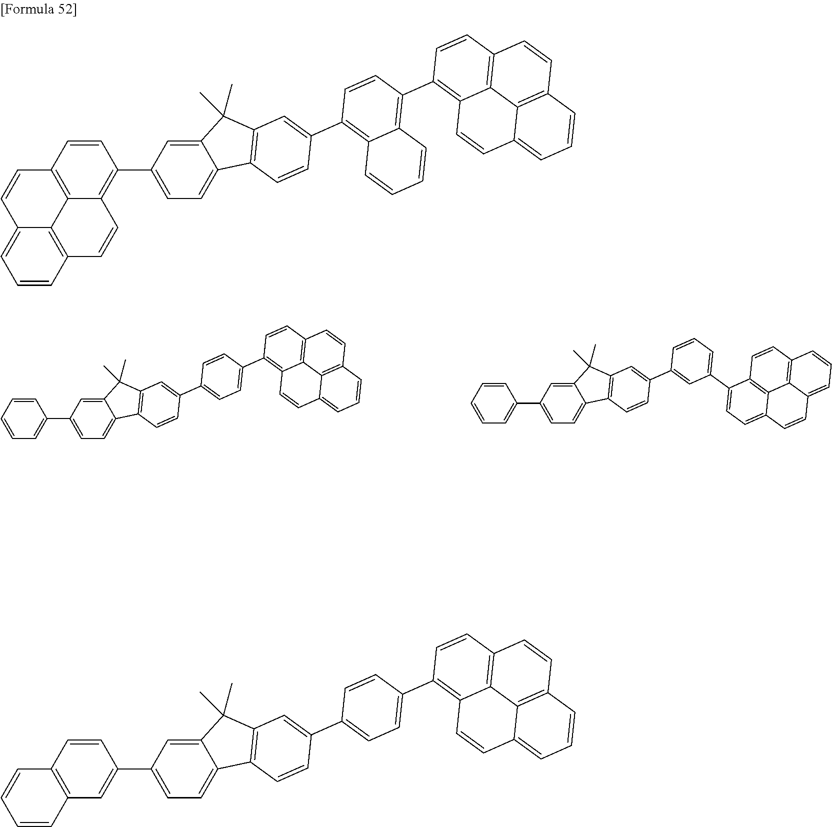

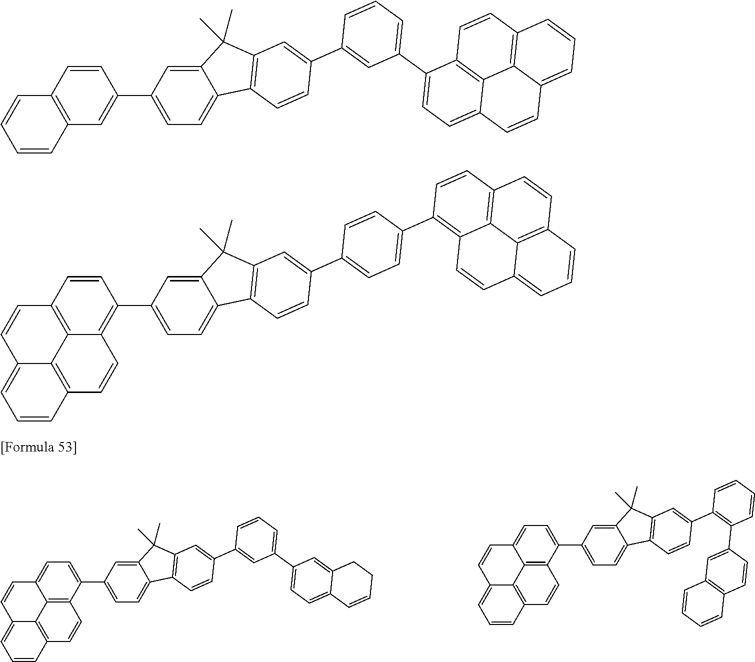

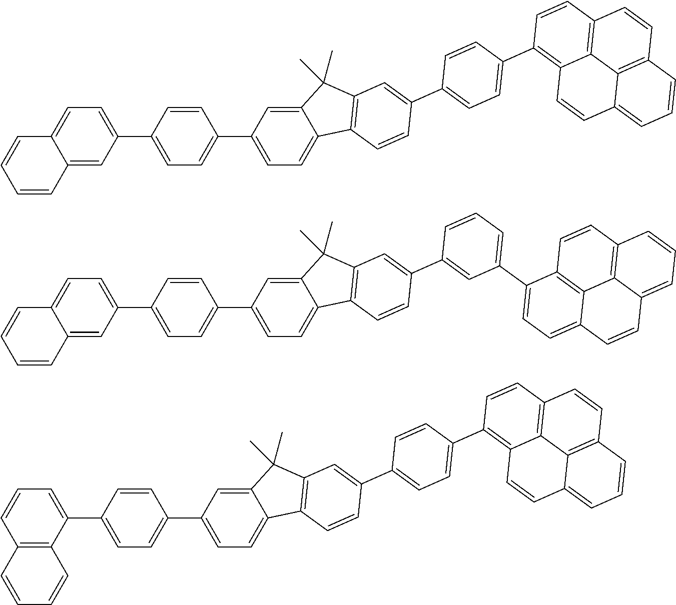

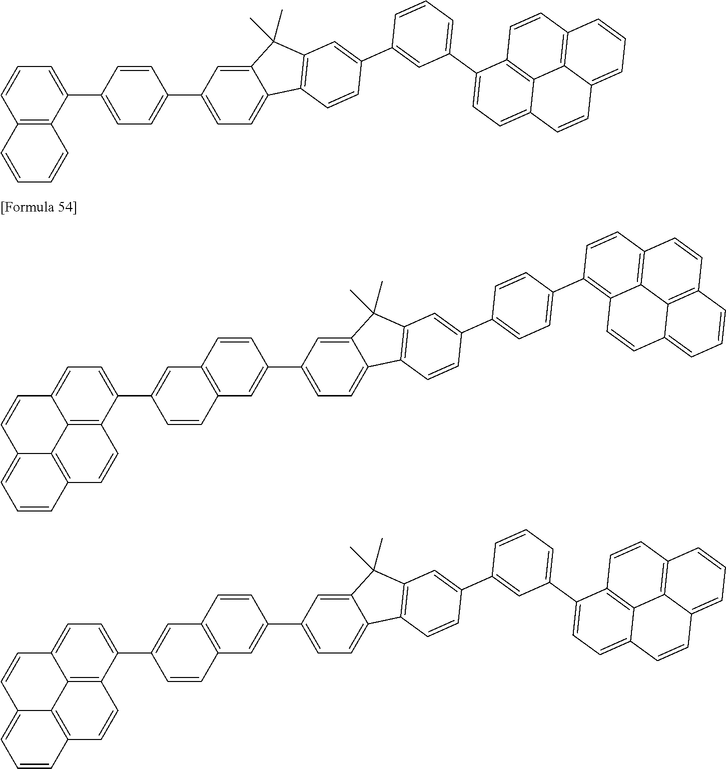

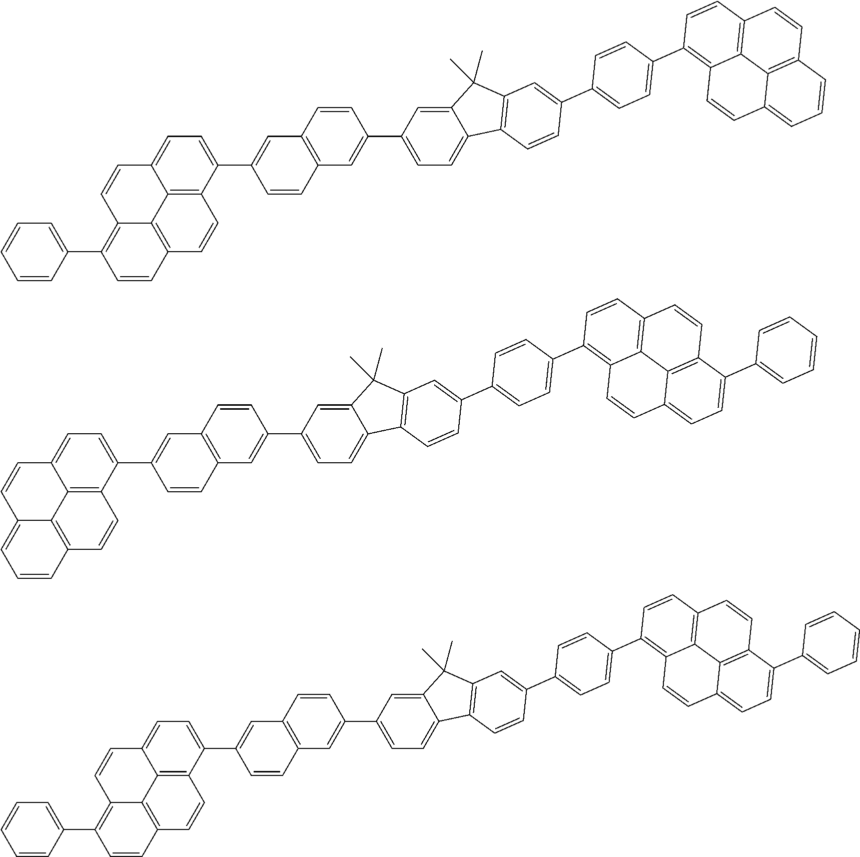

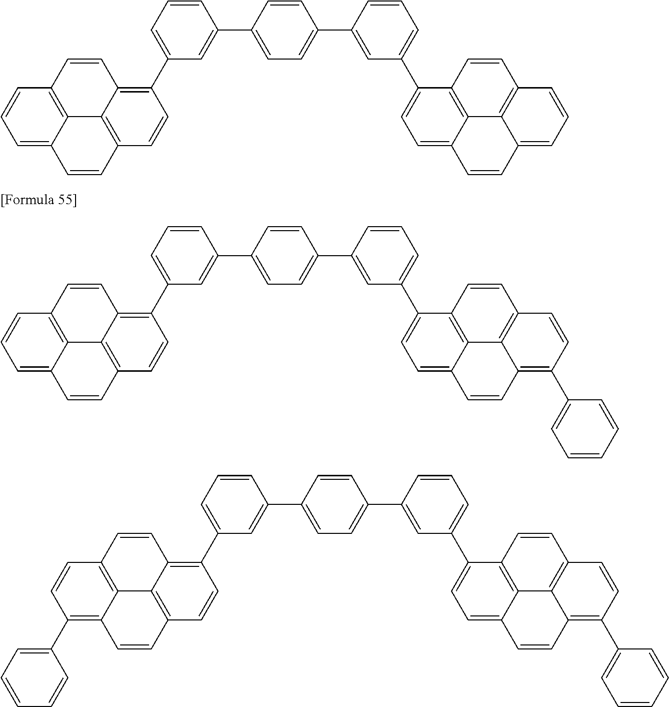

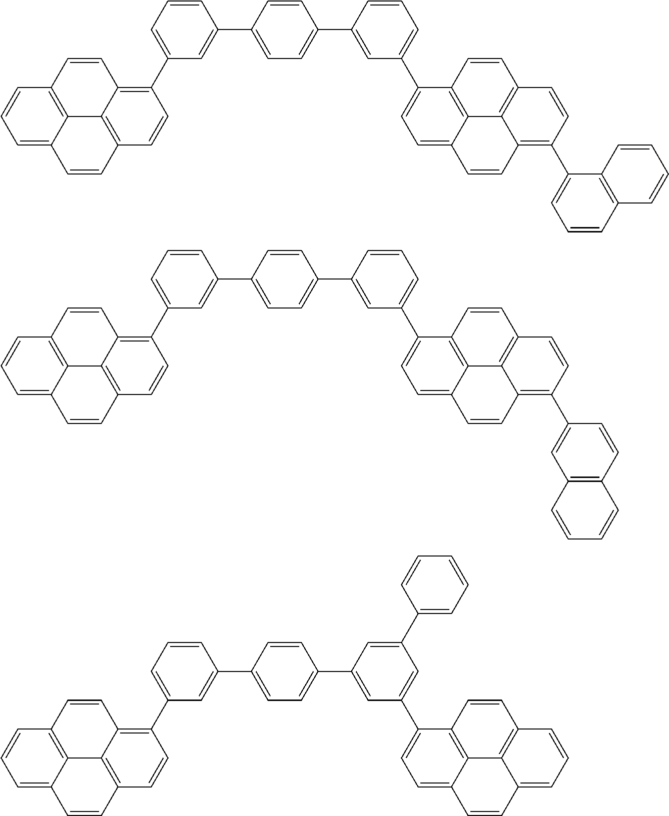

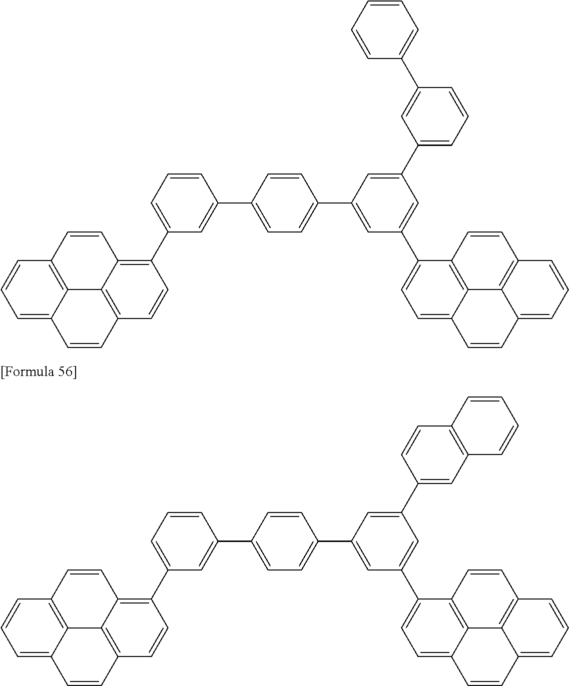

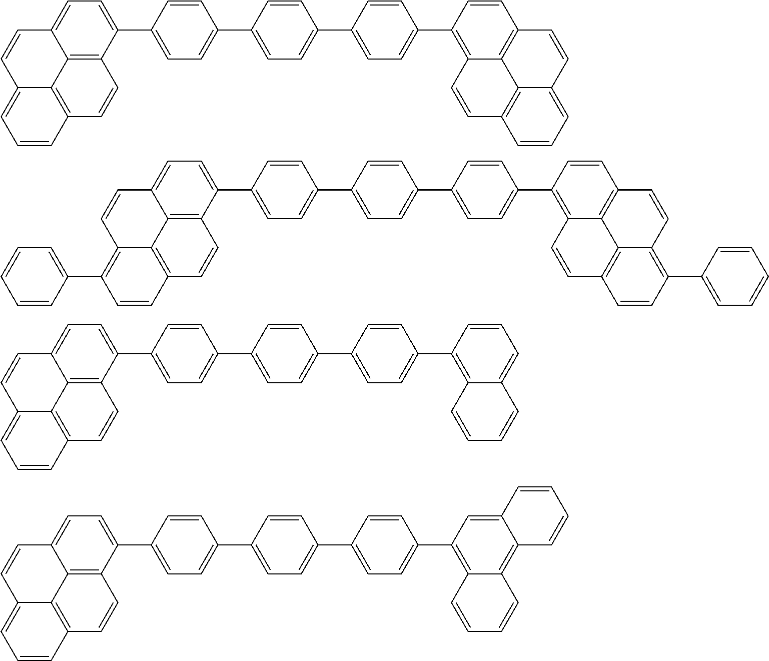

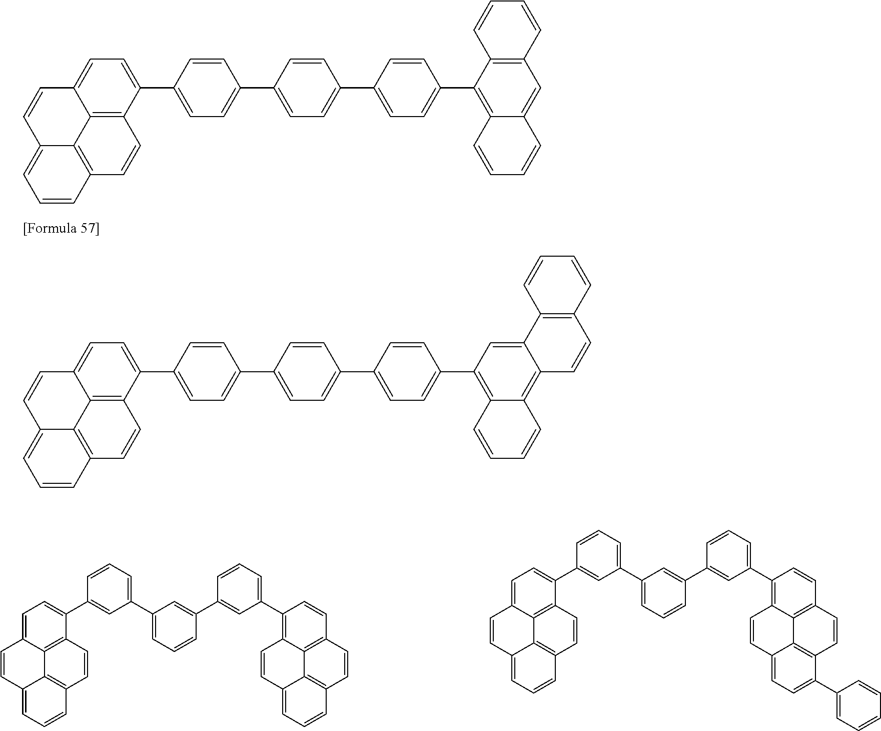

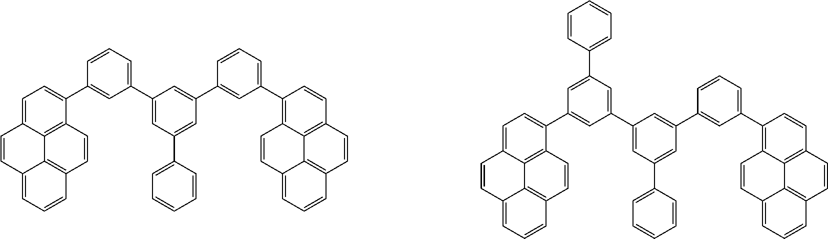

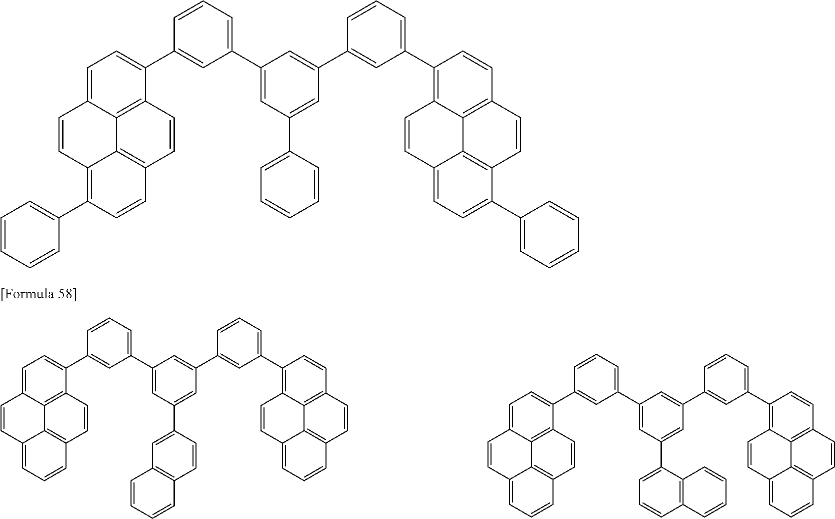

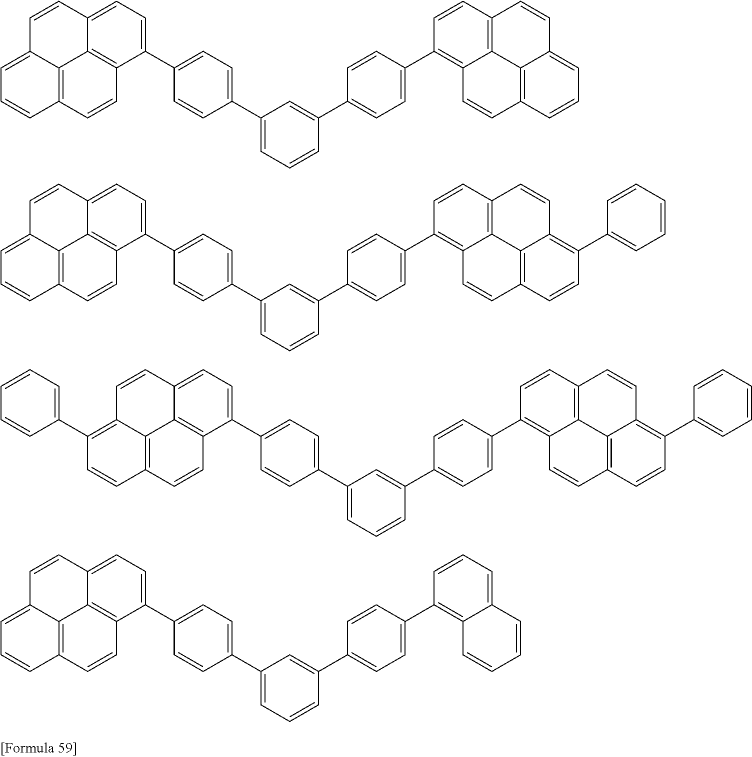

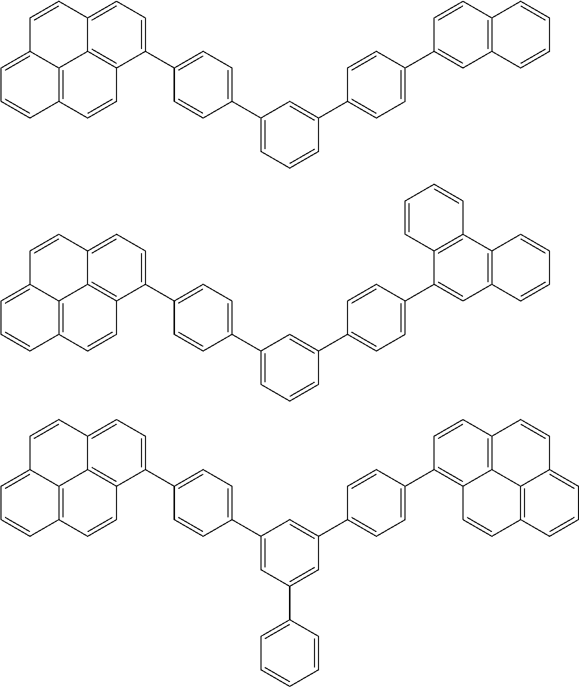

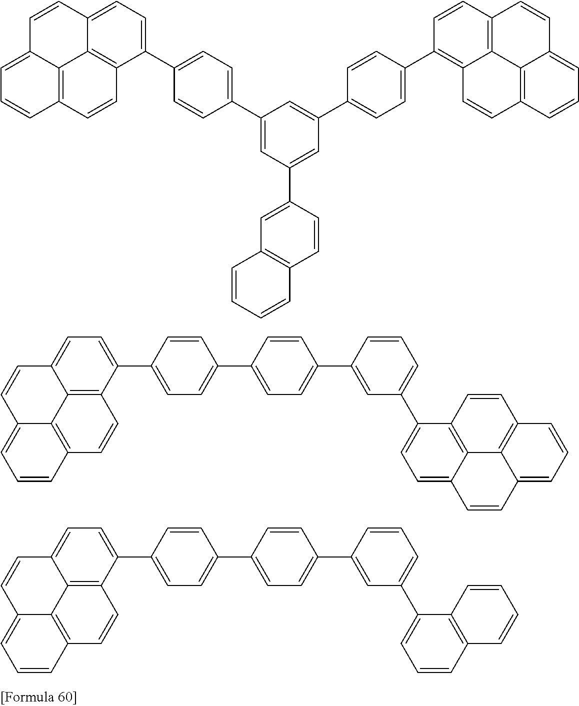

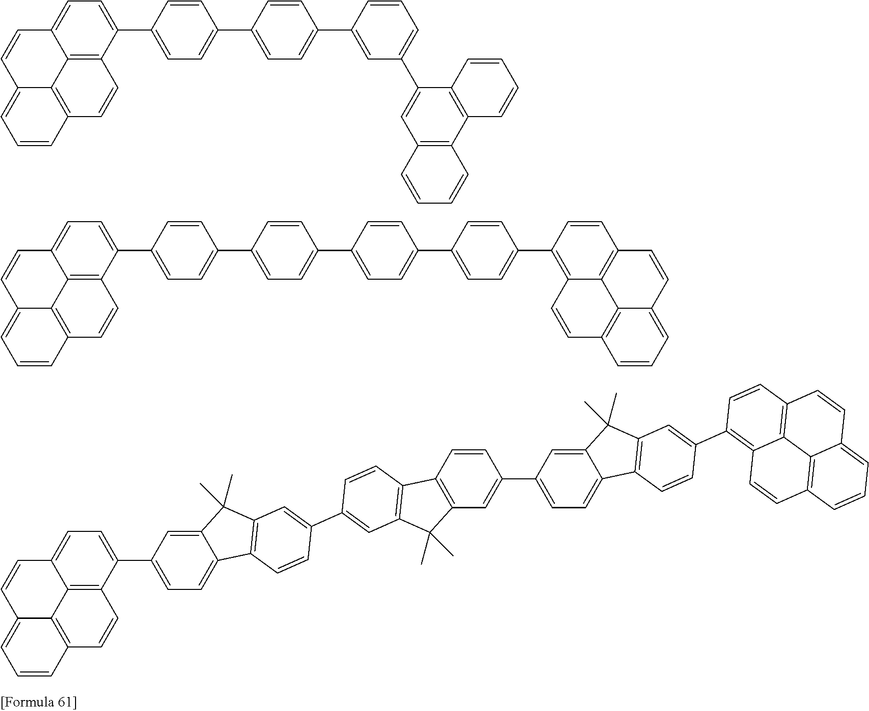

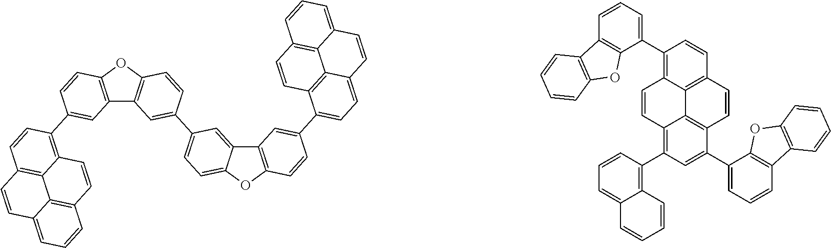 [SMILES: c1cc2ccc3ccc(-c4ccc5oc6ccc(-c7ccc8oc9ccc(-c%10ccc%11ccc%12cccc%13ccc%10c%11c%12%13)cc9c8c7)cc6c5c4)c4ccc(c1)c2c34 ; c1ccc2c(-c3cc(-c4cccc5c4oc4ccccc45)c4ccc5ccc(-c6cccc7c6oc6ccccc67)c6ccc3c4c56)cccc2c1]

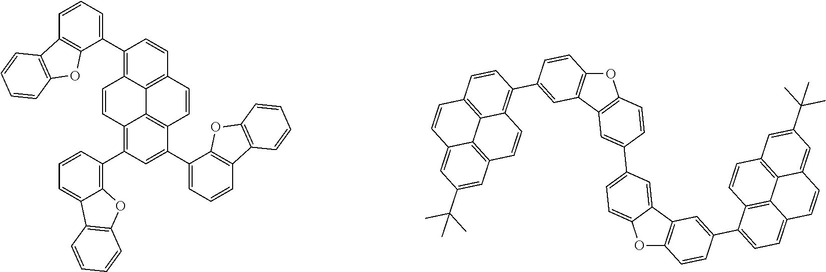 [SMILES: c1ccc2c(c1)oc1c(-c3ccc4ccc5c(-c6cccc7c6oc6ccccc67)cc(-c6cccc7c6oc6ccccc67)c6ccc3c4c56)cccc12 ; CC(C)(C)c1cc2ccc3ccc(-c4ccc5oc6ccc(-c7ccc8oc9ccc(-c%10ccc%11ccc%12cc(C(C)(C)C)cc%13ccc%10c%11c%12%13)cc9c8c7)cc6c5c4)c4ccc(c1)c2c34]

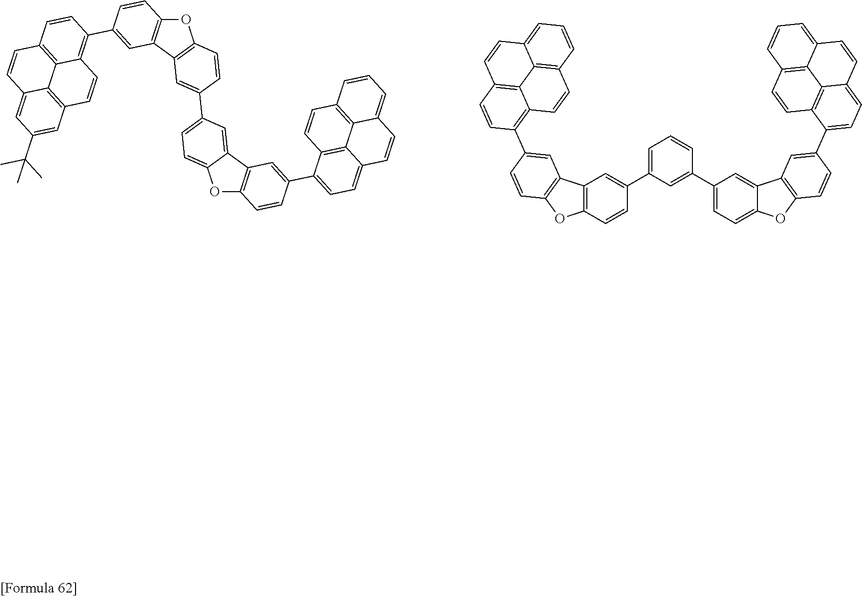 [SMILES: CC(C)(C)c1cc2ccc3ccc(-c4ccc5oc6ccc(-c7ccc8oc9ccc(-c%10ccc%11ccc%12cccc%13ccc%10c%11c%12%13)cc9c8c7)cc6c5c4)c4ccc(c1)c2c34 ; c1cc(-c2ccc3oc4ccc(-c5ccc6ccc7cccc8ccc5c6c78)cc4c3c2)cc(-c2ccc3oc4ccc(-c5ccc6ccc7cccc8ccc5c6c78)cc4c3c2)c1]

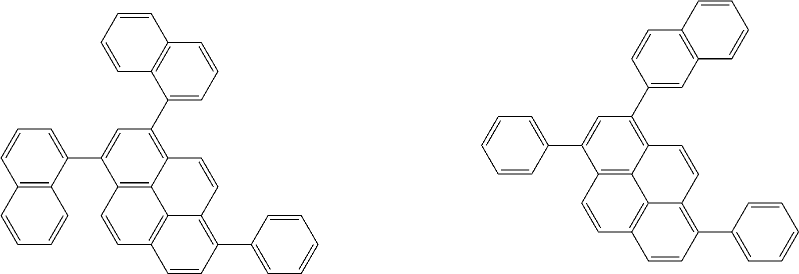

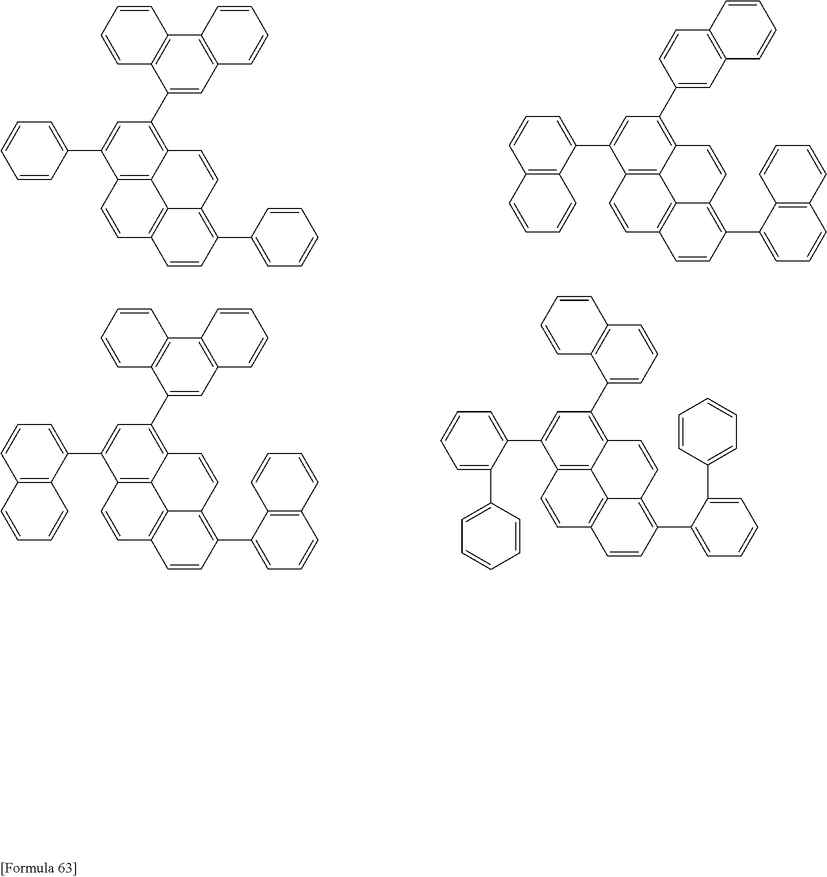

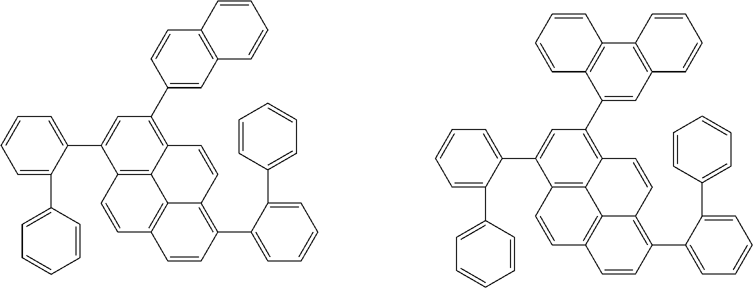

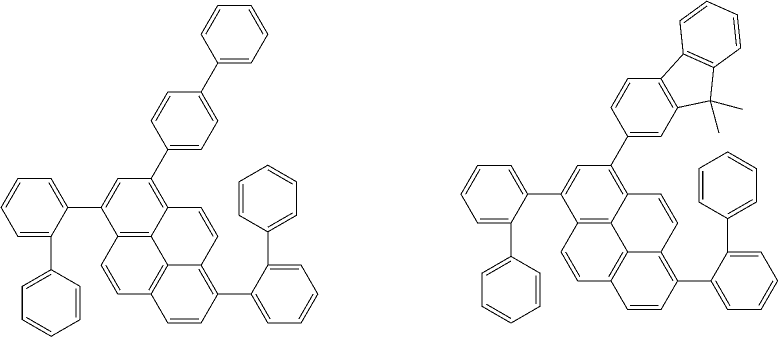

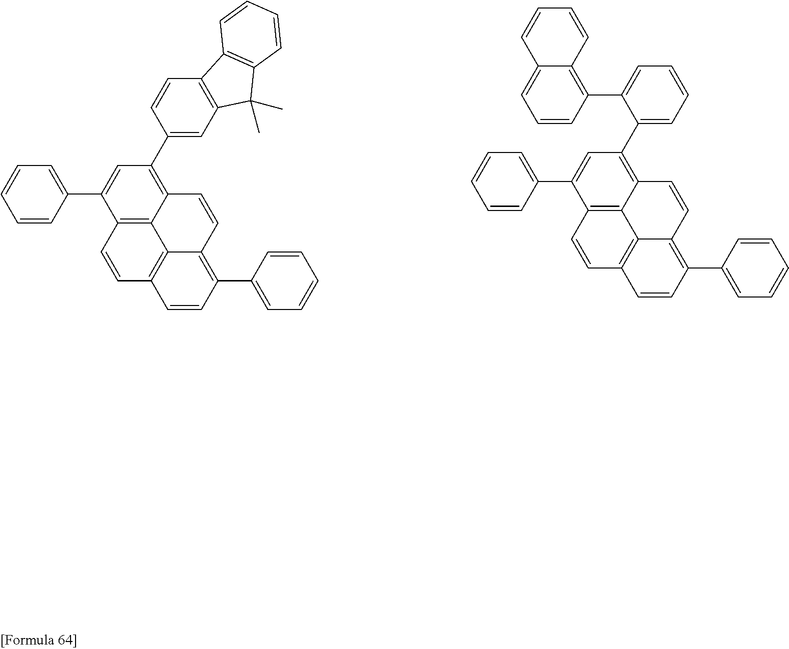

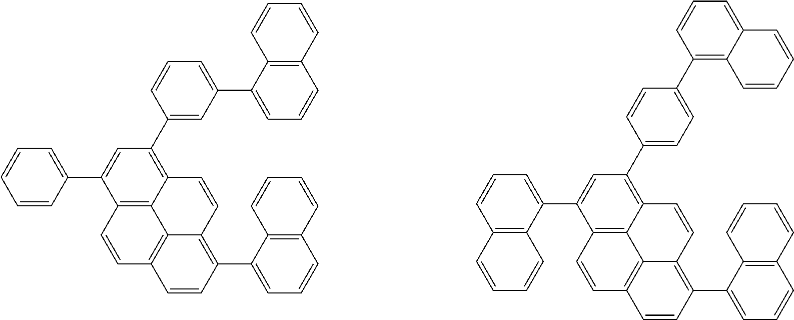

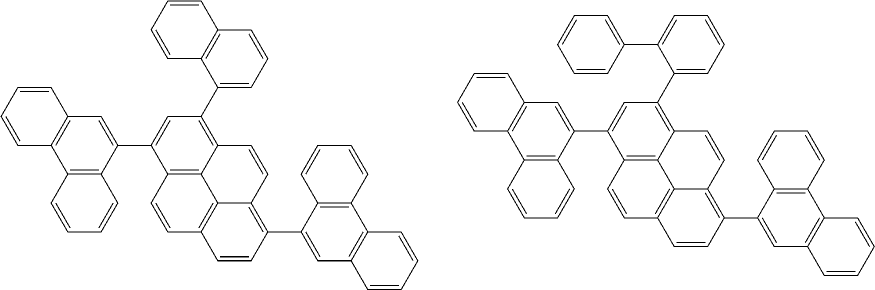

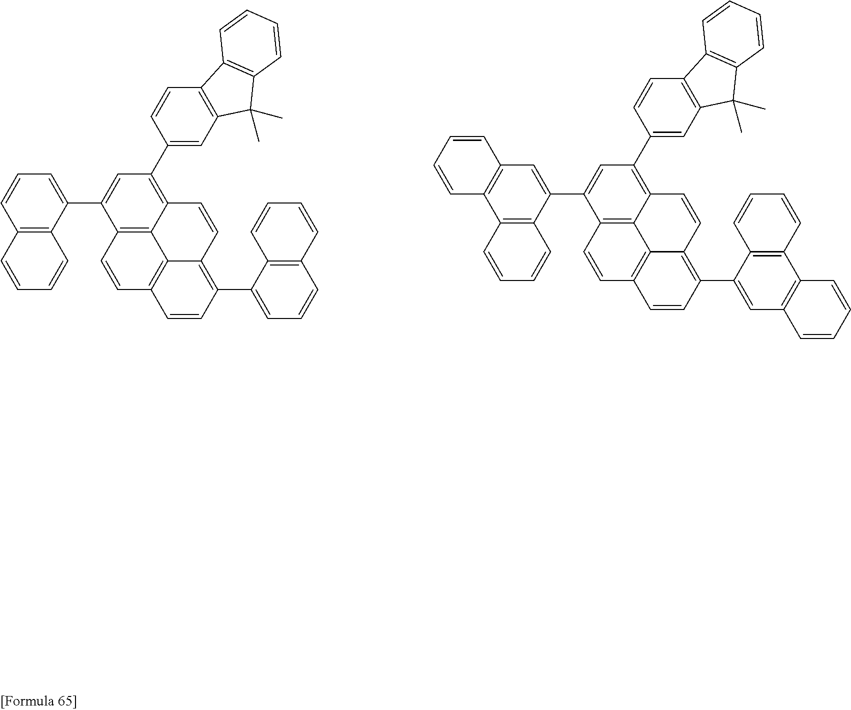

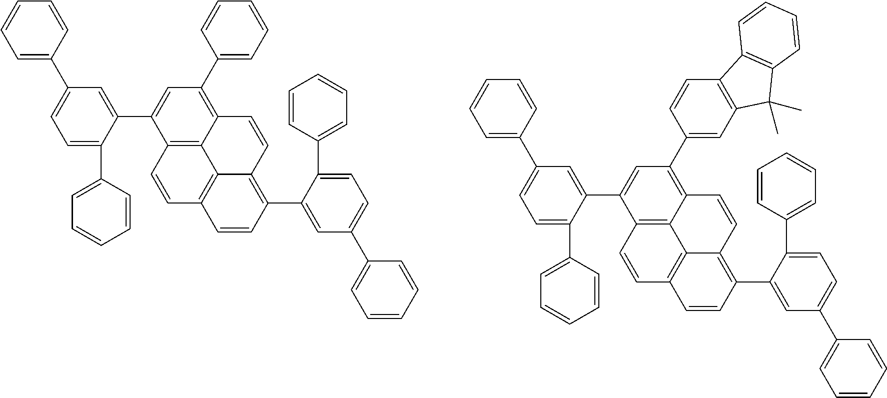

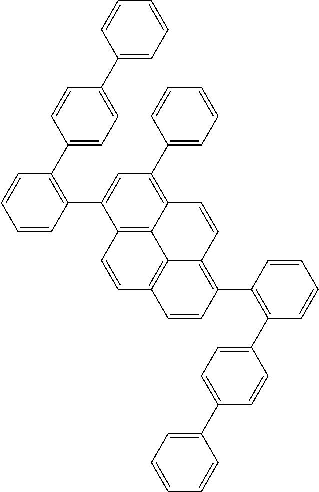 [SMILES: c1ccc(-c2ccc(-c3ccccc3-c3ccc4ccc5c(-c6ccccc6-c6ccc(-c7ccccc7)cc6)cc(-c6ccccc6)c6ccc3c4c65)cc2)cc1]

### Second Compound

In the organic EL device according to the exemplary embodiment, the second compound is a compound represented by a formula (2) below.

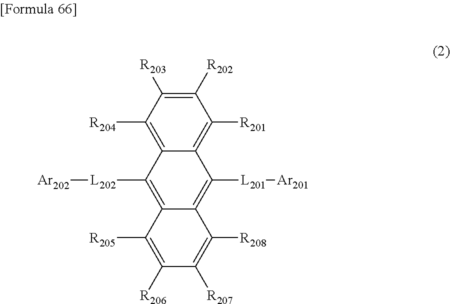

In the formula (2):

R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;

L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and

Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the second compound of the exemplary embodiment: R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₈₀₁ are present, the plurality of R₈₀₁ are mutually the same or different; and

when a plurality of R₈₀₂ are present, the plurality of R₈₀₂ are mutually the same or different.

In the organic EL device according to the exemplary embodiment, it is preferable that: R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, or a nitro group; L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and

Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the organic EL device according to the exemplary embodiment, it is preferable that L₂₀₁ and L₂₀₂ are each independently a single bond, or a substituted or unsubstituted arylene group having 6 to 50 ring carbon atoms; and

Ar₂₀₁ and Ar₂₀₂ are each independently a substituted or unsubstituted aryl group having 6 to 50 ring carbon atoms.

In the organic EL device according to the exemplary embodiment, it is preferable that Ar₂₀₁ and Ar₂₀₂ are each independently a phenyl group, a naphthyl group, a phenanthryl group, a biphenyl group, a terphenyl group, a diphenylfluorenyl group, a dimethylfluorenyl group, a benzodiphenylfluorenyl group, a benzodimethylfluorenyl group, a dibenzofuranyl group, a dibenzothienyl group, a naphthobenzofuranyl group, or a naphthobenzothienyl group.

In the organic EL device according to the exemplary embodiment, the second compound represented by the formula (2) is preferably a compound represented by a formula (201), (202), (203), (204), (205), (206), (207), (208) or (209) below.

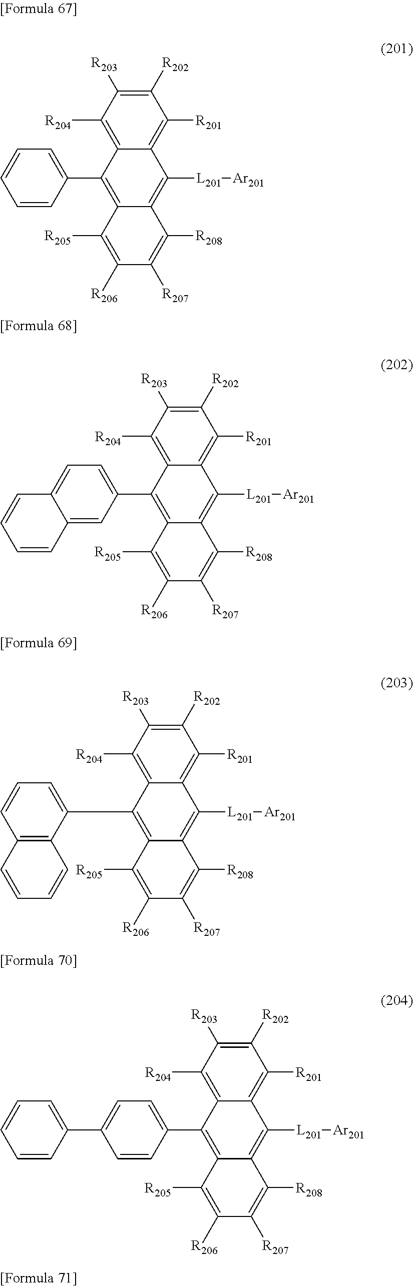

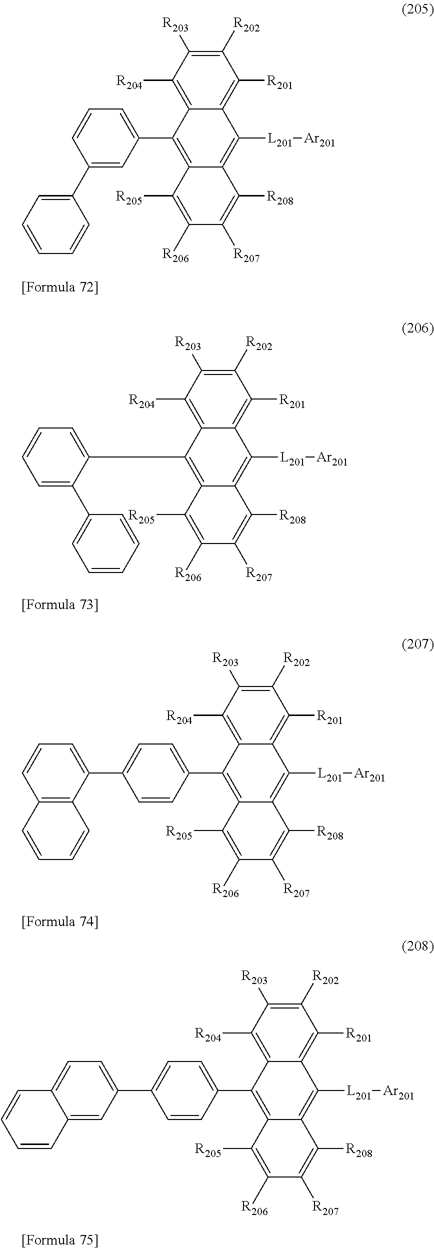

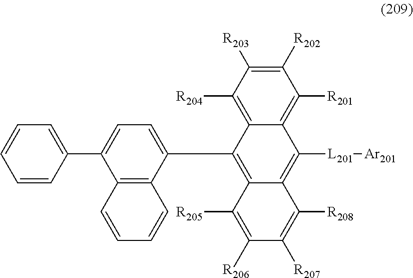

In the formulae (201) to (209):

L₂₀₁ and Ar₂₀₁ represent the same as L₂₀₁ and Ar₂₀₁ in the formula (2); and

R₂₀₁ to R₂₀₈ each independently represent the same as R₂₀₁ to R₂₀₈ in the formula (2).

The second compound represented by the formula (2) is also preferably a compound represented by a formula (221), (222), (223), (224), (225), (226), (227), (228) or (229) below.

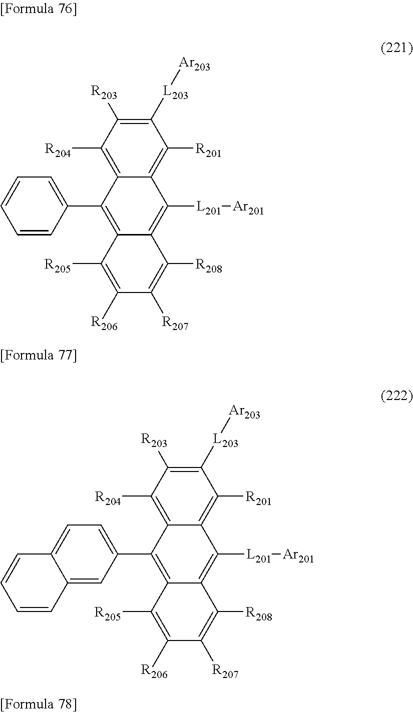

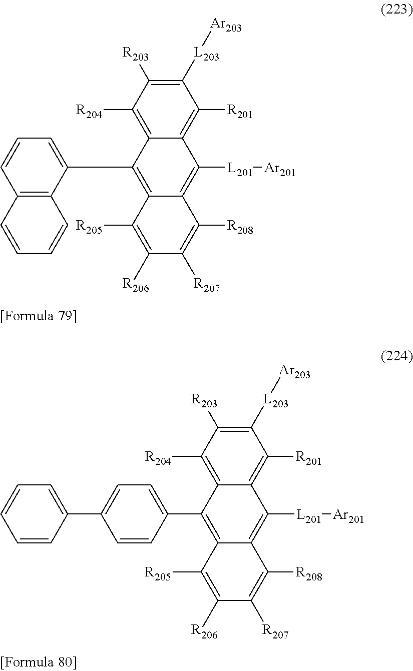

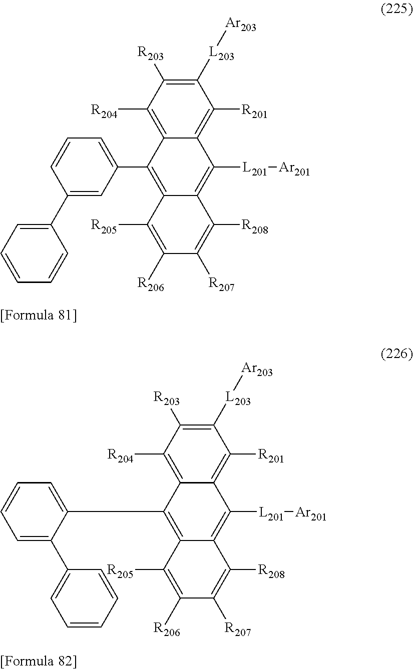

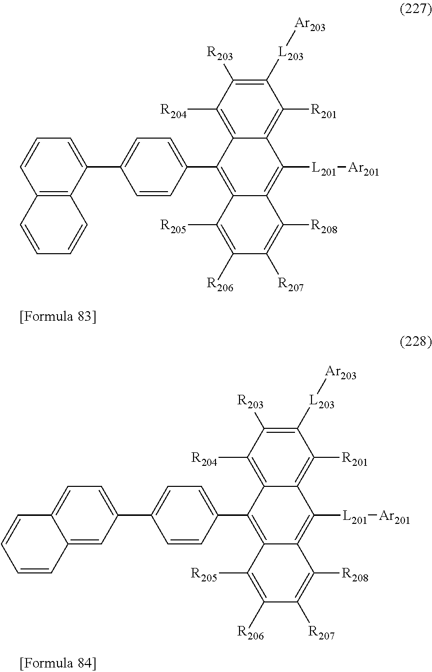

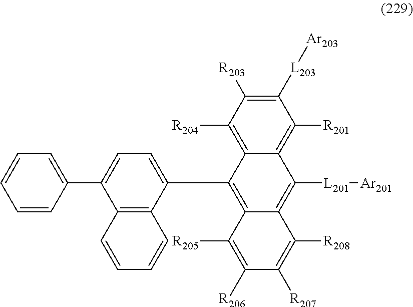

In the formulae (221), (222), (223), (224), (225), (226), (227), (228) and (229):

R₂₀₁ and R₂₀₃ to R₂₀₈ each independently represent the same as R₂₀₁ and R₂₀₃ to R₂₀₈ in the formula (2);

L₂₀₁ and Ar₂₀₁ respectively represent the same as L₂₀₁ and Ar₂₀₁ in the formula (2);

L₂₀₃ represents the same as L₂₀₁ in the formula (2);

L₂₀₃ and L₂₀₁ are mutually the same or different;

Ar₂₀₃ represents the same as Ar₂₀₁ in the formula (2); and

Ar₂₀₃ and Ar₂₀₁ are mutually the same or different.

The second compound represented by the formula (2) is also preferably a compound represented by a formula (241), (242), (243), (244), (245), (246), (247), (248) or (249) below.

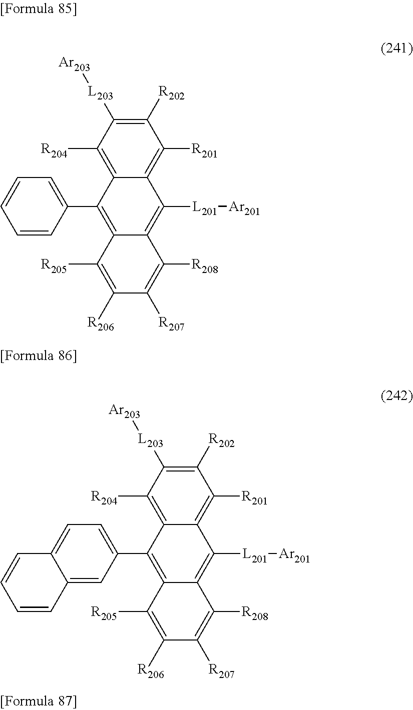

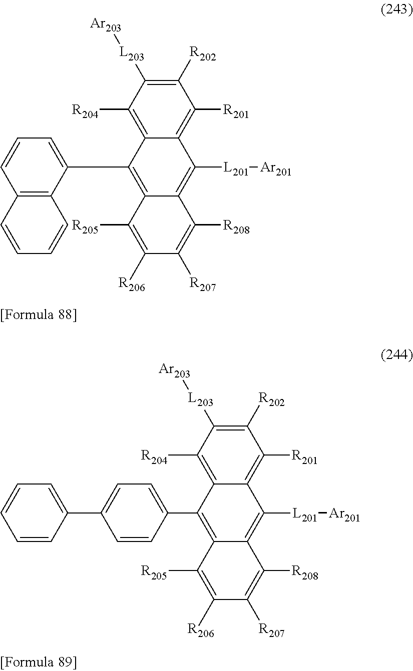

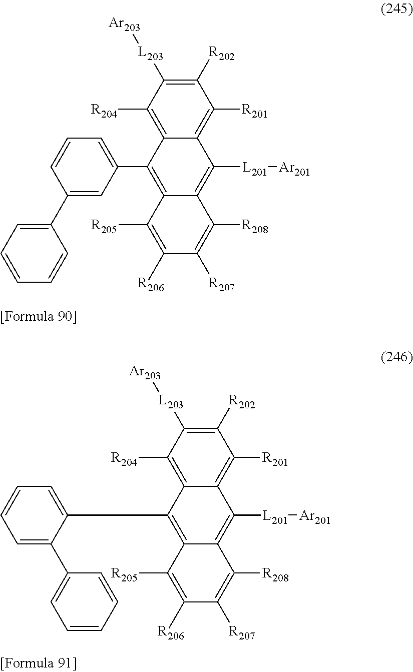

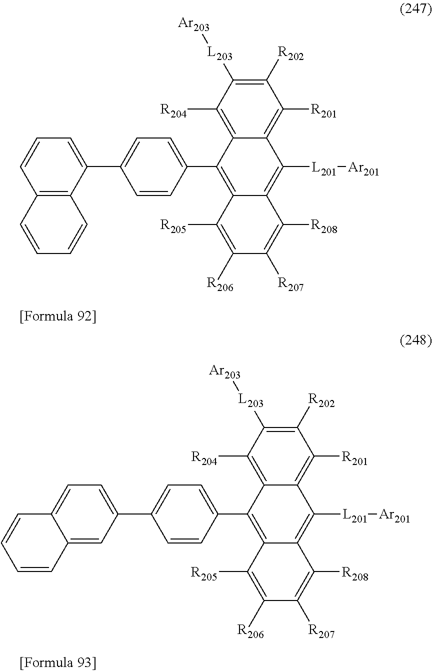

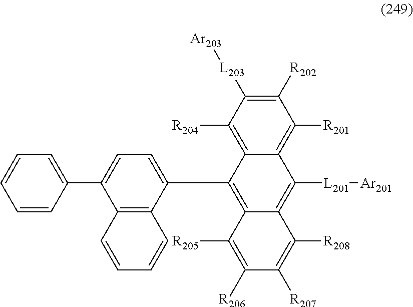

In the formulae (241), (242), (243), (244), (245), (246), (247), (248) and (249):

R₂₀₁, R₂₀₂ and R₂₀₄ to R₂₀₈ each independently represent the same as R₂₀₁, R₂₀₂ and R₂₀₄ to R₂₀₈ in the formula (2);

L₂₀₃ represents the same as L₂₀₁ in the formula (2);

L₂₀₃ and L₂₀₁ are mutually the same or different;

Ar₂₀₃ represents the same as Ar₂₀₁ in the formula (2); and

Ar₂₀₃ and Ar₂₀₁ are mutually the same or different.

In the second compound represented by the formula (2), it is preferable that R₂₀₁ to R₂₀₈ not being a group represented by a formula (21) are each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

L₁₀₁ is preferably a single bond, or an unsubstituted arylene group having 6 to 22 ring carbon atoms; and Ar₁₀₁ is preferably a substituted or unsubstituted aryl group having 6 to 22 ring carbon atoms.

In the organic EL device according to the exemplary embodiment, R₂₀₁ to R₂₀₈ that are substituents on an anthracene skeleton in the second compound represented by the formula (2) are preferably hydrogen atoms in terms of preventing inhibition of intermolecular interaction to inhibit a decrease in electron mobility. However, R₂₀₁ to R₂₀₈ may be a substituted or unsubstituted aryl group having 6 to 50 ring carbon atoms or a substituted or unsubstituted heterocyclic group having 5 to 50 ring atoms.

Assuming that R₂₀₁ to R₂₀₈ each are a bulky substituent such as an alkyl group and a cycloalkyl group, intermolecular interaction may be inhibited to decrease the electron mobility of the second compound relative to that of the first compound, so that a relationship of pH2>μH1 shown by a numerical formula below (Numerical Formula 3) may not be satisfied. When the second compound is used in the second emitting layer, it can be expected that satisfying the relationship of pH2>μH1 inhibits a decrease in a recombination ability between holes and electrons in the first emitting layer and a decrease in a luminous efficiency. It should be noted that substituents, namely, a haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group are likely to be bulky, and an alkyl group and cycloalkyl group are likely to be further bulky.

In the second compound represented by the formula (2), R₂₀₁ to R₂₀₈, which are the substituents on the anthracene skeleton, are each preferably not a bulky substituent and preferably not an alkyl group and cycloalkyl group. More preferably, R₂₀₁ to R₂₀₈ are not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the organic EL device according to the exemplary embodiment, it is also preferable that R₂₀₁ to R₂₀₈ in the second compound represented by the formula (2) are each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In the organic EL device according to the exemplary embodiment, it is preferable that R₂₀₁ to R₂₀₈ in the second compound represented by the formula (2) are each a hydrogen atom.

In the second compound, examples of the substituent for a “substituted or unsubstituted group” on R₂₀₁ to R₂₀₈ also preferably do not include the above-described substituent that is likely to be bulky, especially a substituted or unsubstituted alkyl group and a substituted or unsubstituted cycloalkyl group. Since examples of the substituent for a “substituted or unsubstituted” group on R₂₀₁ to R₂₀₈ do not include a substituted or unsubstituted alkyl group and a substituted or unsubstituted cycloalkyl group, inhibition of intermolecular interaction to be caused by presence of a bulky substituent such as an alkyl group and a cycloalkyl group can be prevented, thereby preventing a decrease in the electron mobility. Moreover, when the second compound described above is used in the second emitting layer, a decrease in a recombination ability between holes and electrons in the first emitting layer and a decrease in the luminous efficiency can be inhibited.

It is more preferable that R₂₀₁ to R₂₀₈, which are the substituents on the anthracene skeleton, are not bulky substituents, and R₂₀₁ to R₂₀₈ as substituents are unsubstituted. Assuming that R₂₀₁ to R₂₀₈, which are the substituents on the anthracene skeleton, are not bulky substituents and substituents are bonded to R₂₀₁ to R₂₀₈ which are the not-bulky substituents, the substituents bonded to R₂₀₁ to R₂₀₈ are preferably not the bulky substituents; the substituents bonded to R₂₀₁ to R₂₀₈ serving as substituents are preferably not an alkyl group and cycloalkyl group, more preferably not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the second compound, all groups defined to be “substituted or unsubstituted” are each preferably an “unsubstituted” group.

In the organic EL device according to the exemplary embodiment, for instance, Ar₂₀₁ in the second compound represented by the formula (2) is a substituted or unsubstituted dibenzofuranyl group.

In the organic EL device according to the exemplary embodiment, for instance, Ar₂₀₁ in the second compound represented by the formula (2) is an unsubstituted dibenzofuranyl group.

In the organic EL device according to the exemplary embodiment, for instance, the second compound represented by the formula (2) has at least one hydrogen atom, the hydrogen atom including at least one deuterium atom.

In the organic EL device according to the exemplary embodiment, for instance, L₂₀₁ in the second compound represented by the formula (2) is one of TEMP-63 to TEMP-68.

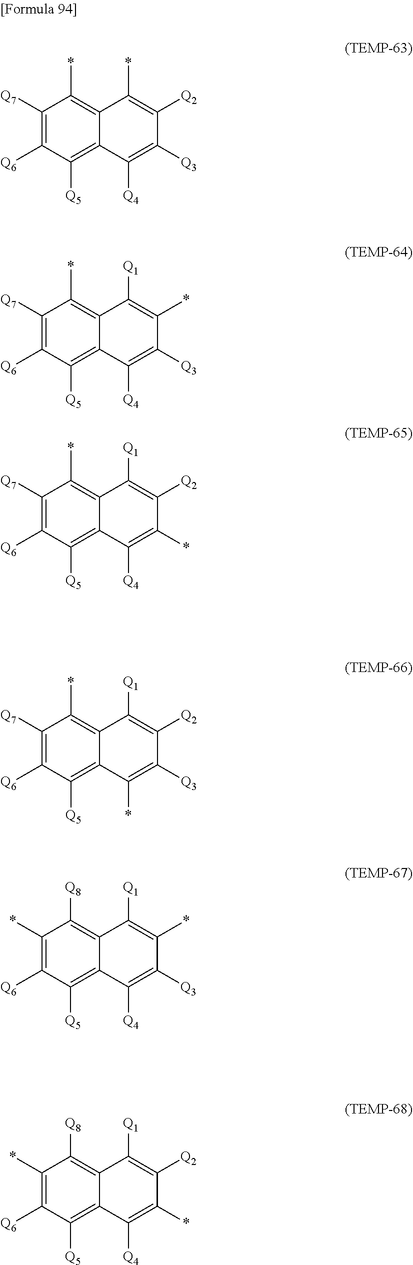

In the organic EL device according to the exemplary embodiment, for instance, Ar₂₀₁ in the second compound represented by the formula (2) is at least one group selected from the group consisting of substituted or unsubstituted anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluoranthenyl group, benzofluoranthenyl group, and perylenyl group.

In the organic EL device according to the exemplary embodiment, for instance, Ar₂₀₁ in the second compound represented by the formula (2) is a substituted or unsubstituted fluorenyl group.

In the organic EL device according to the exemplary embodiment, for instance, Ar₂₀₁ in the second compound represented by the formula (2) is a substituted or unsubstituted xanthenyl group.

In the organic EL device according to the exemplary embodiment, for instance, Ar₂₀₁ in the second compound represented by the formula (2) is a benzoxanthenyl group.

### Manufacturing Method of Second Compound

The second compound can be manufactured by a known method. The second compound can also be manufactured based on a known method through a known alternative reaction using a known material(s) tailored for the target compound.

### Specific Examples of Second Compound

Specific examples of the second compound include the following compounds. It should however be noted that the invention is not limited to the specific examples of the second compound.

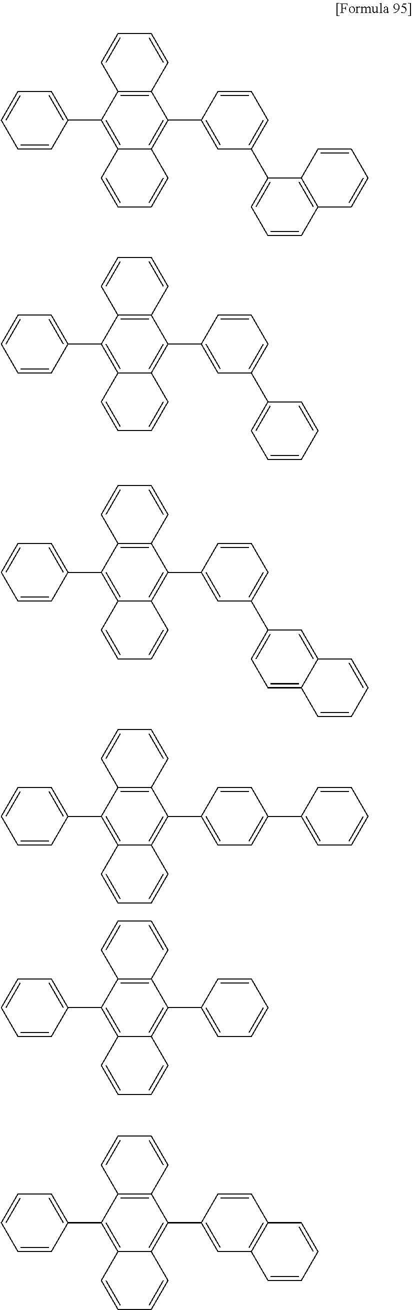

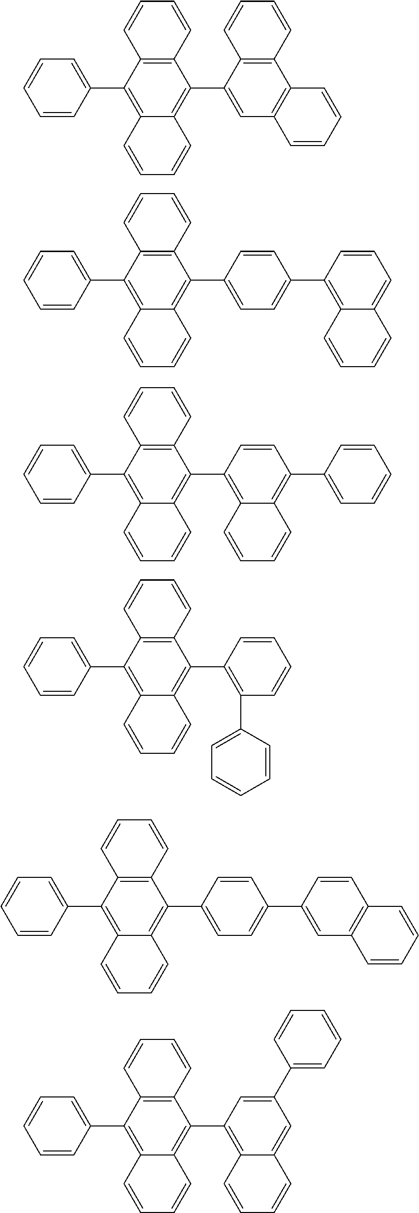

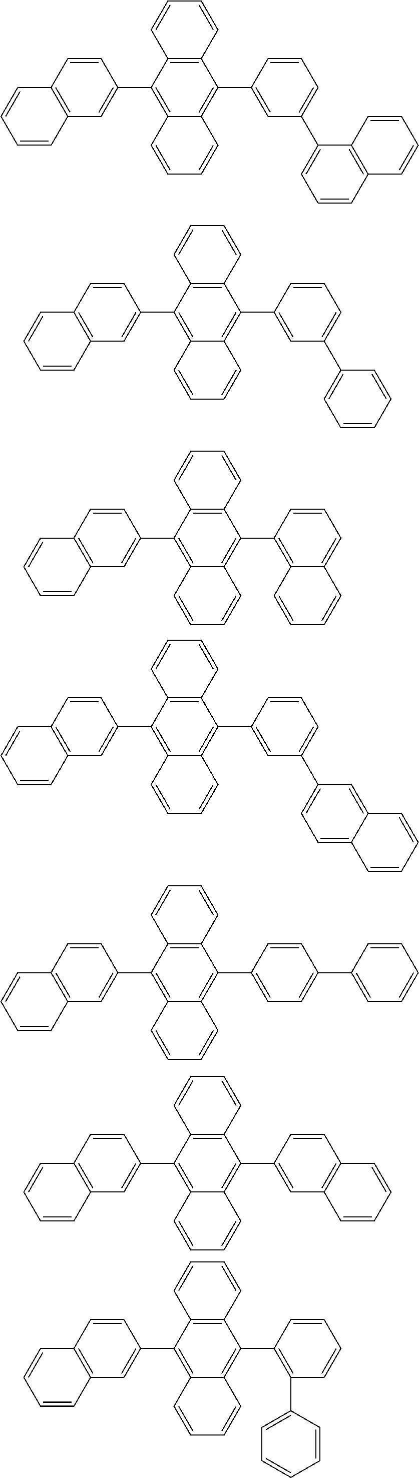

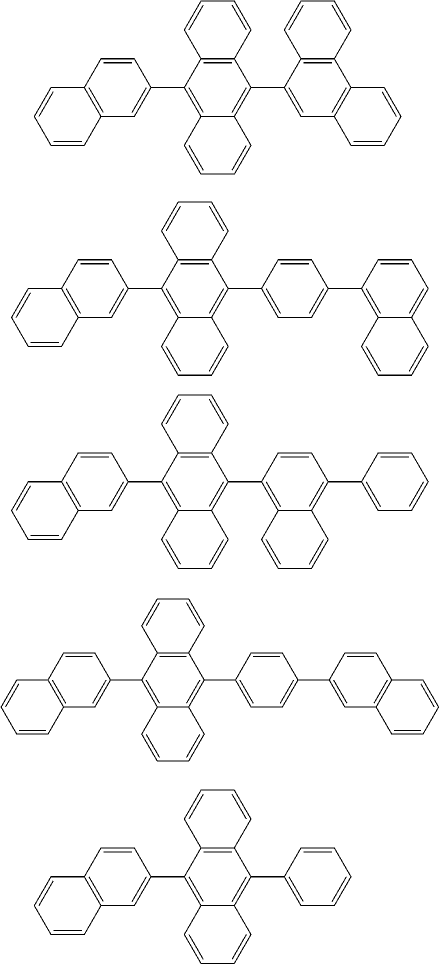

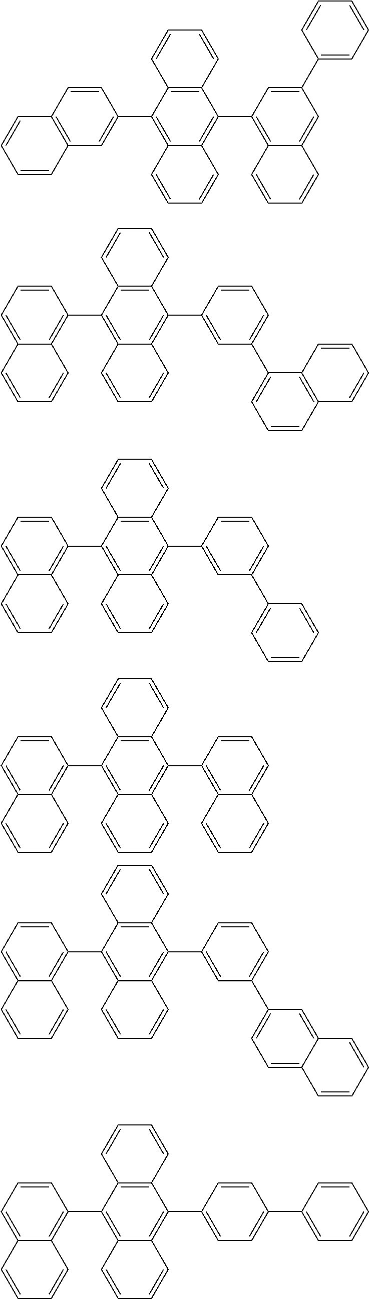

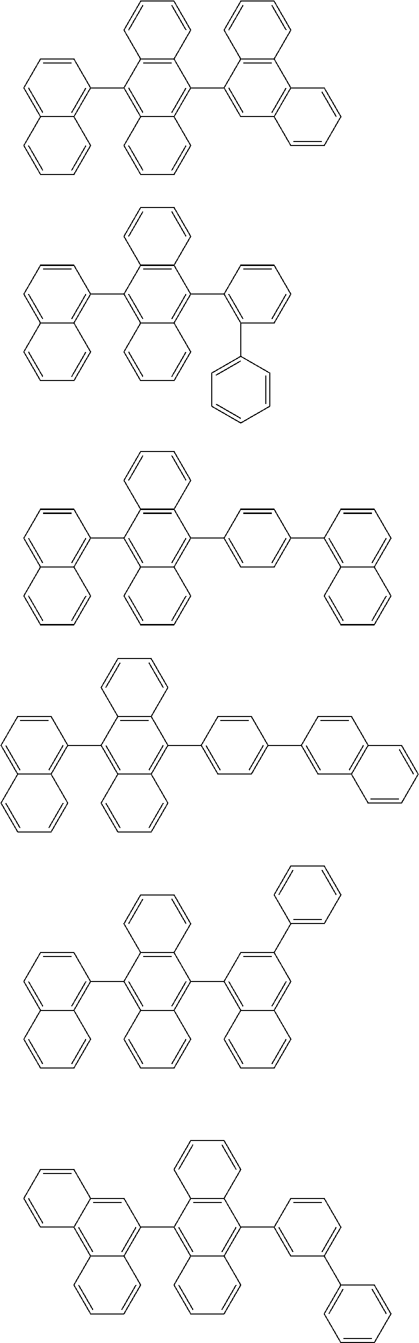

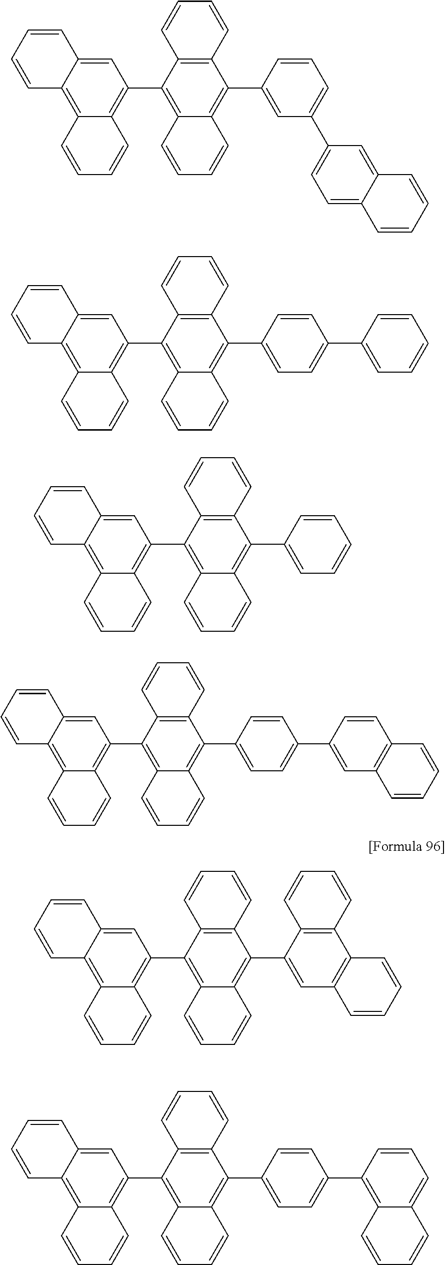

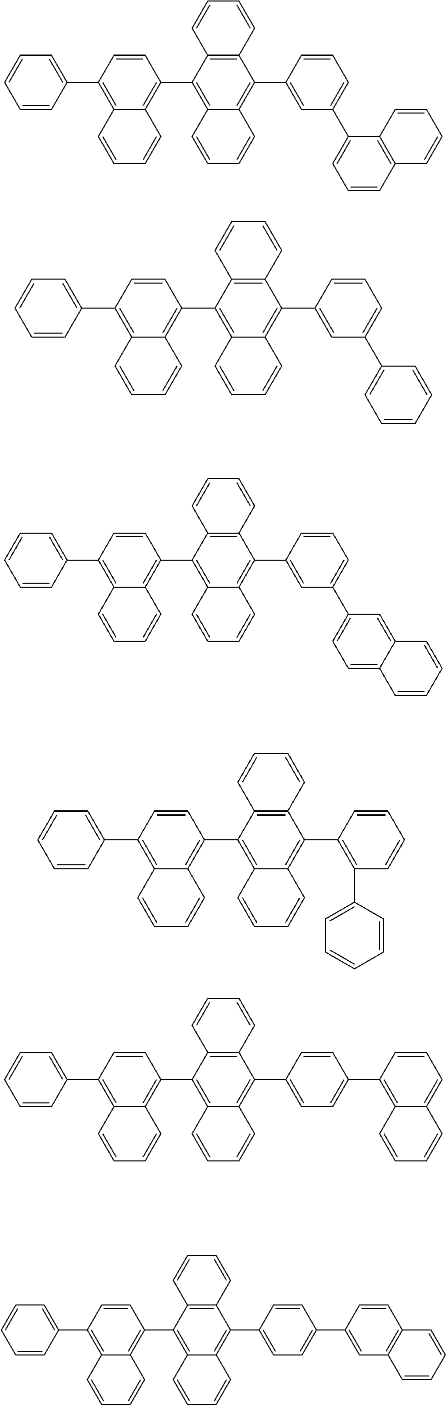

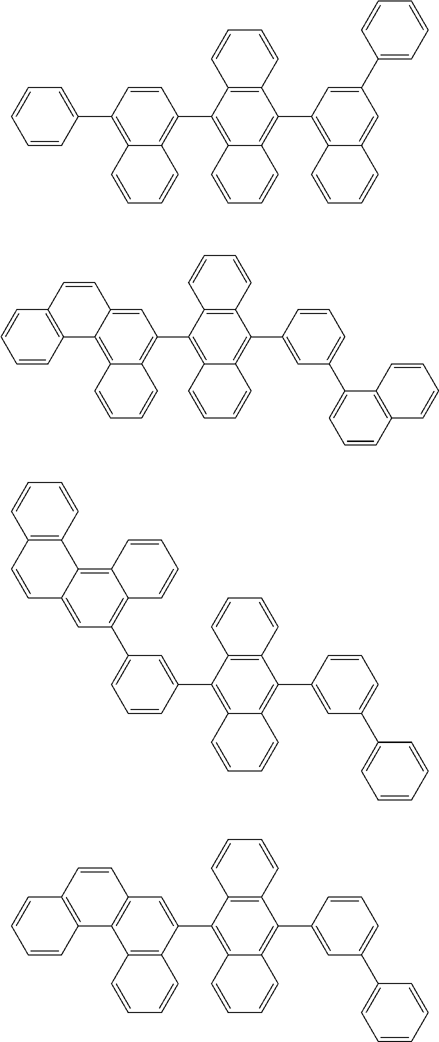

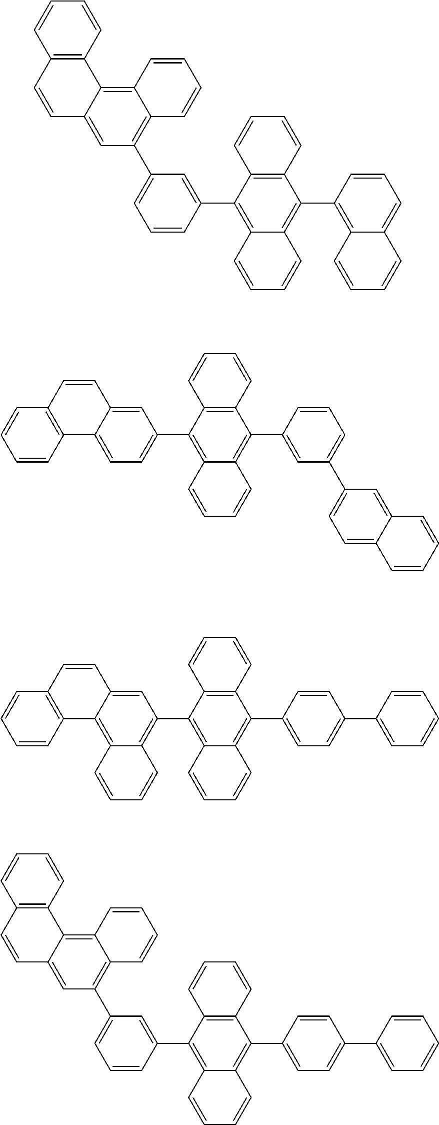

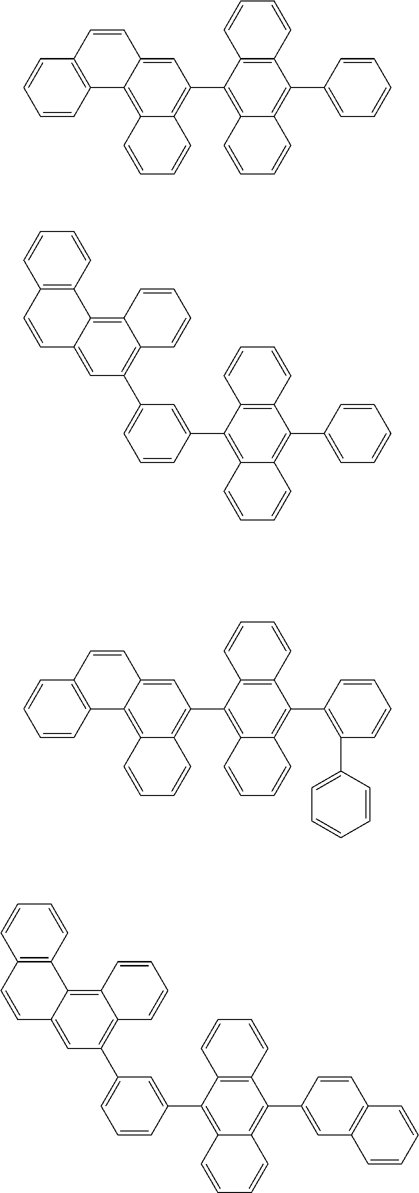

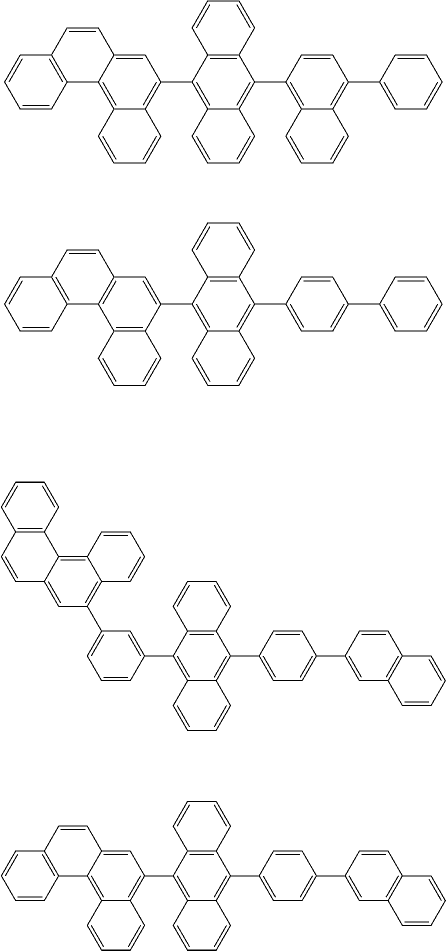

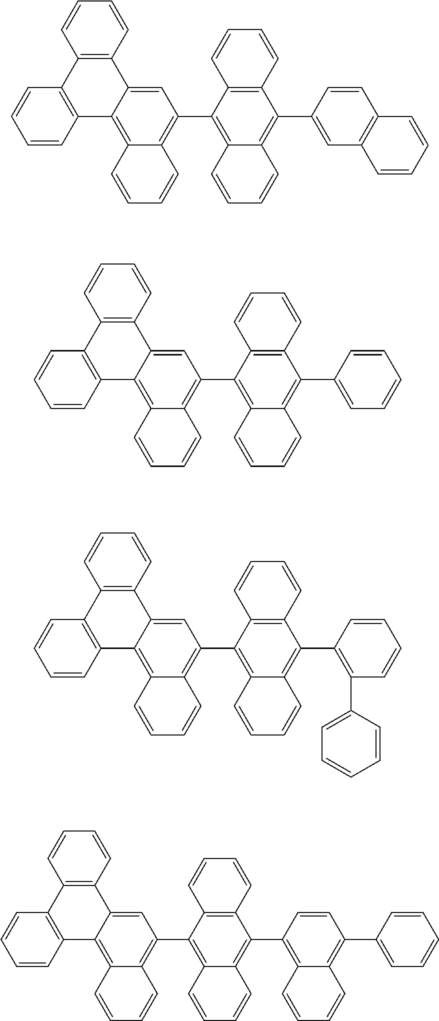

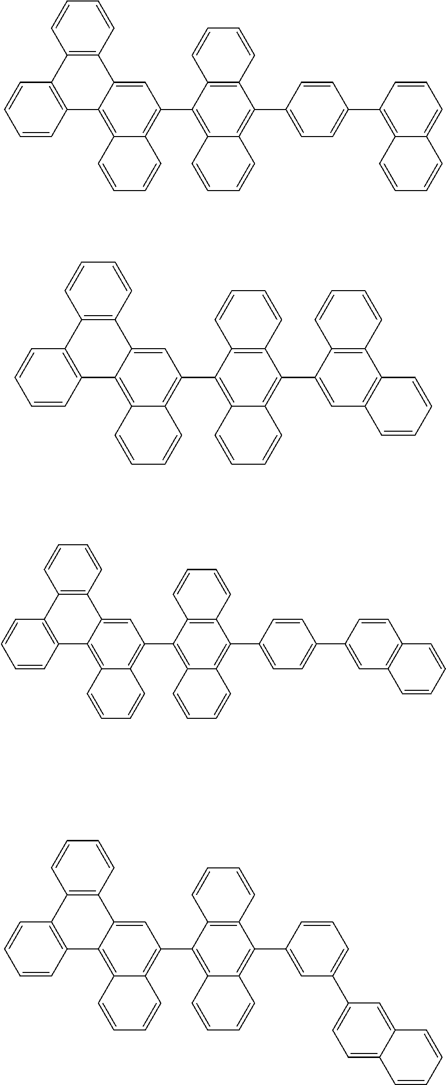

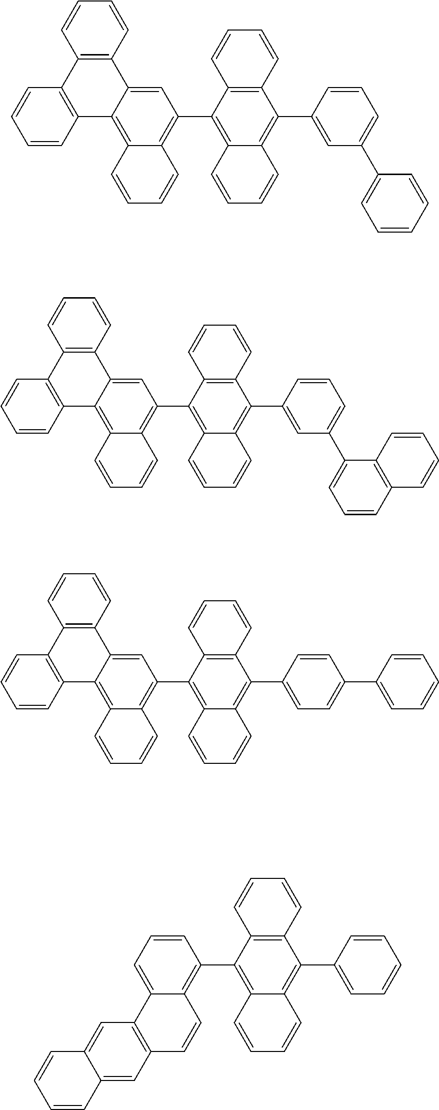

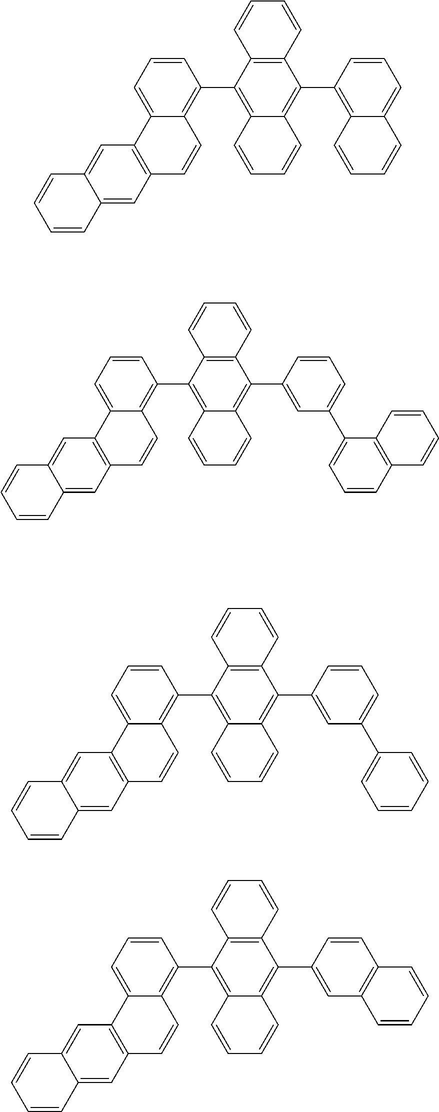

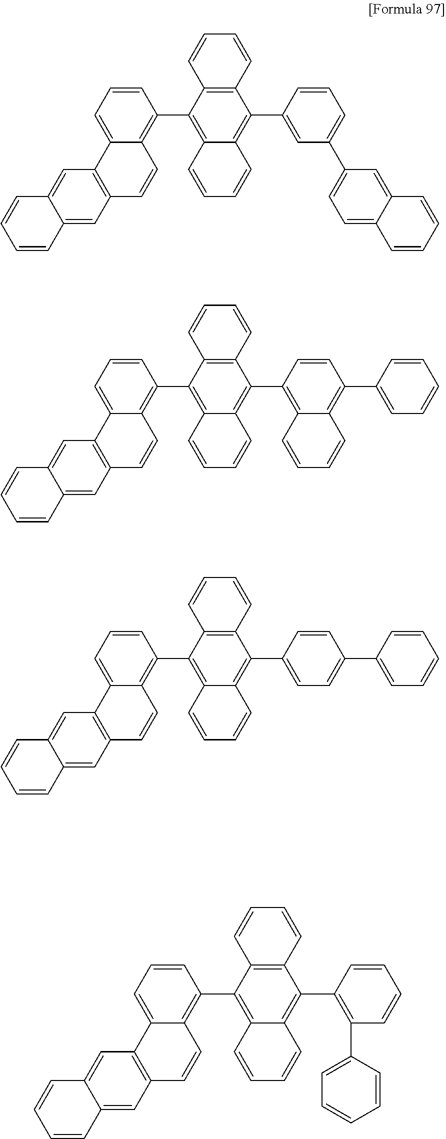

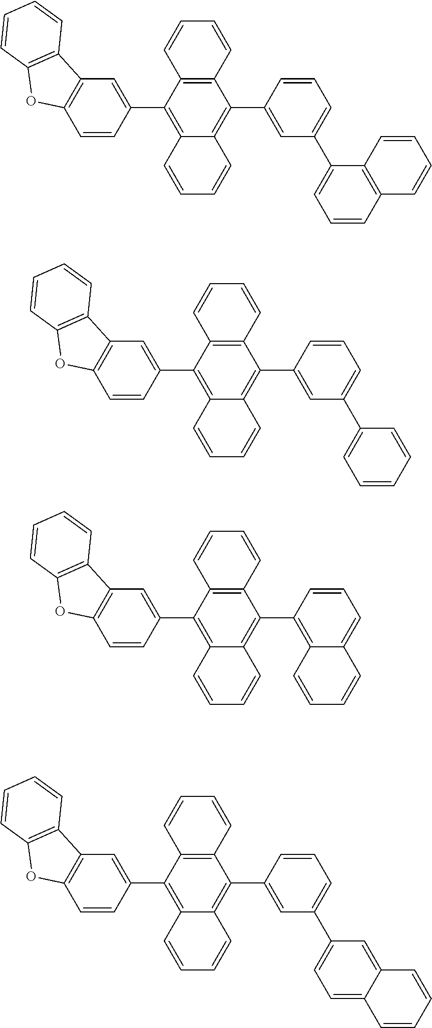 [SMILES: c1cc(-c2cccc3ccccc23)cc(-c2c3ccccc3c(-c3ccc4oc5ccccc5c4c3)c3ccccc23)c1 ; c1ccc(-c2cccc(-c3c4ccccc4c(-c4ccc5oc6ccccc6c5c4)c4ccccc34)c2)cc1 ; c1ccc2c(-c3c4ccccc4c(-c4ccc5oc6ccccc6c5c4)c4ccccc34)cccc2c1 ; c1cc(-c2ccc3ccccc3c2)cc(-c2c3ccccc3c(-c3ccc4oc5ccccc5c4c3)c3ccccc23)c1]

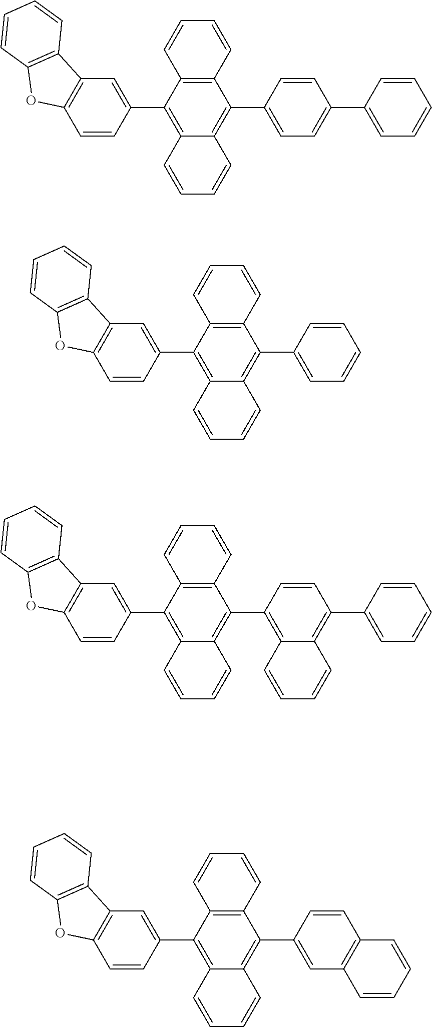 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1 ; c1ccc(-c2ccc(-c3c4ccccc4c(-c4ccc5oc6ccccc6c5c4)c4ccccc34)c3ccccc23)cc1 ; c1ccc2cc(-c3c4ccccc4c(-c4ccc5oc6ccccc6c5c4)c4ccccc34)ccc2c1]

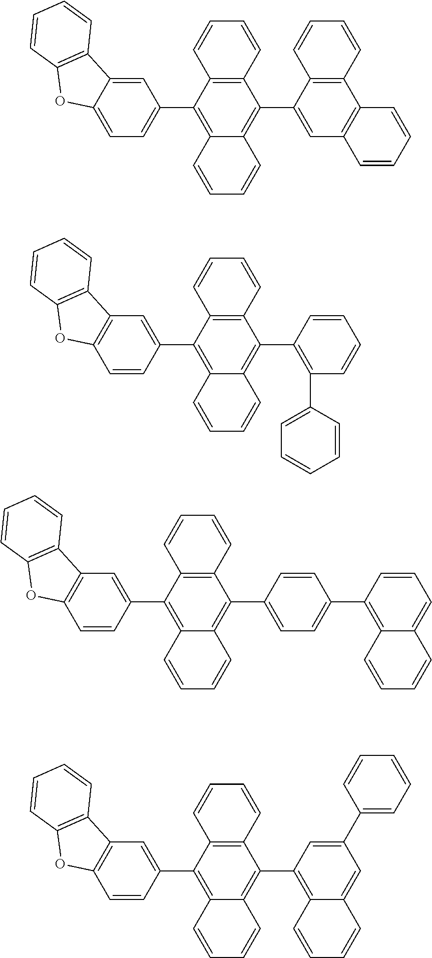 [SMILES: c1ccc2c(c1)cc(-c1c3ccccc3c(-c3ccc4oc5ccccc5c4c3)c3ccccc13)c1ccccc12 ; c1ccc(-c2ccccc2-c2c3ccccc3c(-c3ccc4oc5ccccc5c4c3)c3ccccc23)cc1 ; c1ccc2c(-c3ccc(-c4c5ccccc5c(-c5ccc6oc7ccccc7c6c5)c5ccccc45)cc3)cccc2c1 ; c1ccc(-c2cc(-c3c4ccccc4c(-c4ccc5oc6ccccc6c5c4)c4ccccc34)c3ccccc3c2)cc1]

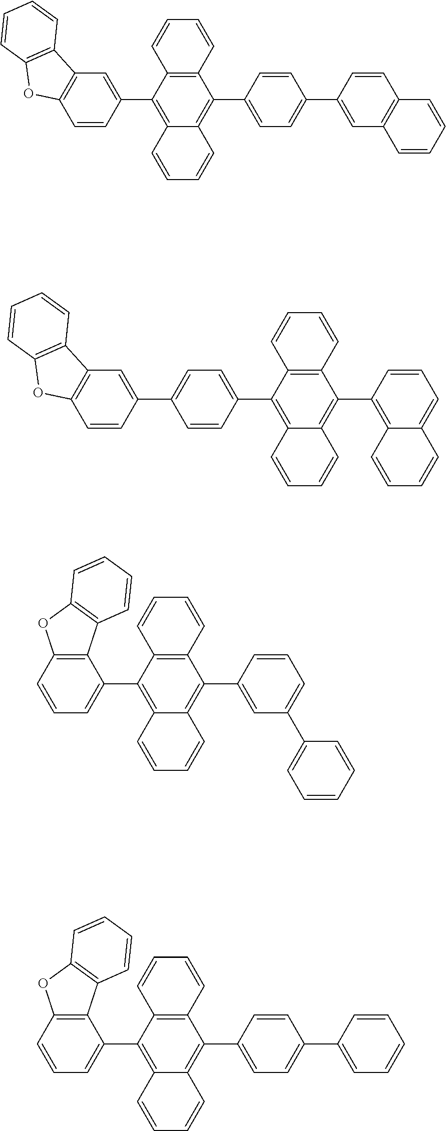 [SMILES: c1ccc2cc(-c3ccc(-c4c5ccccc5c(-c5ccc6oc7ccccc7c6c5)c5ccccc45)cc3)ccc2c1 ; c1ccc2c(-c3c4ccccc4c(-c4ccc(-c5ccc6oc7ccccc7c6c5)cc4)c4ccccc34)cccc2c1 ; c1ccc(-c2cccc(-c3c4ccccc4c(-c4cccc5oc6ccccc6c45)c4ccccc34)c2)cc1 ; c1ccc(-c2ccc(-c3c4ccccc4c(-c4cccc5oc6ccccc6c45)c4ccccc34)cc2)cc1]

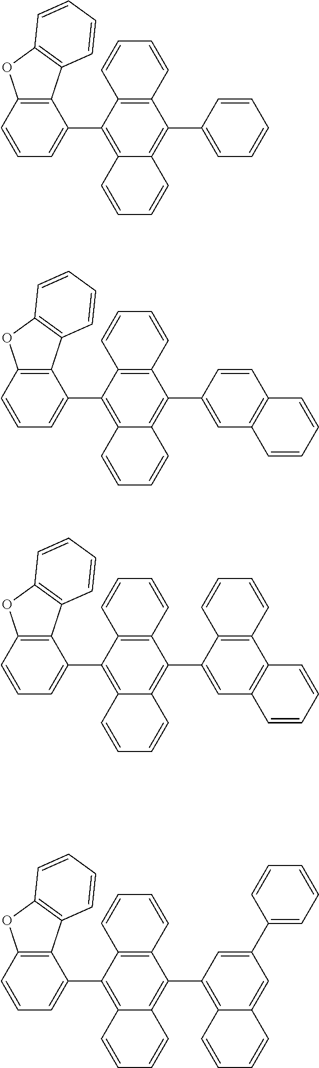 [SMILES: c1ccc(-c2c3ccccc3c(-c3cccc4oc5ccccc5c34)c3ccccc23)cc1 ; c1ccc2cc(-c3c4ccccc4c(-c4cccc5oc6ccccc6c45)c4ccccc34)ccc2c1 ; c1ccc2c(c1)cc(-c1c3ccccc3c(-c3cccc4oc5ccccc5c34)c3ccccc13)c1ccccc12 ; c1ccc(-c2cc(-c3c4ccccc4c(-c4cccc5oc6ccccc6c45)c4ccccc34)c3ccccc3c2)cc1]

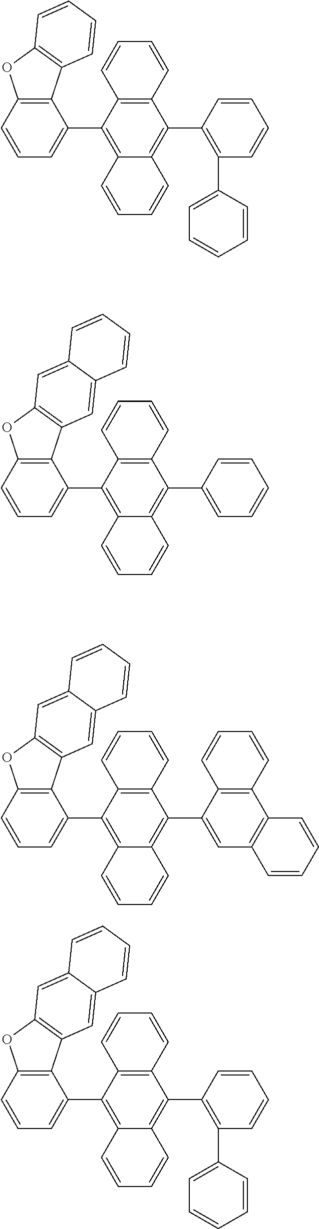

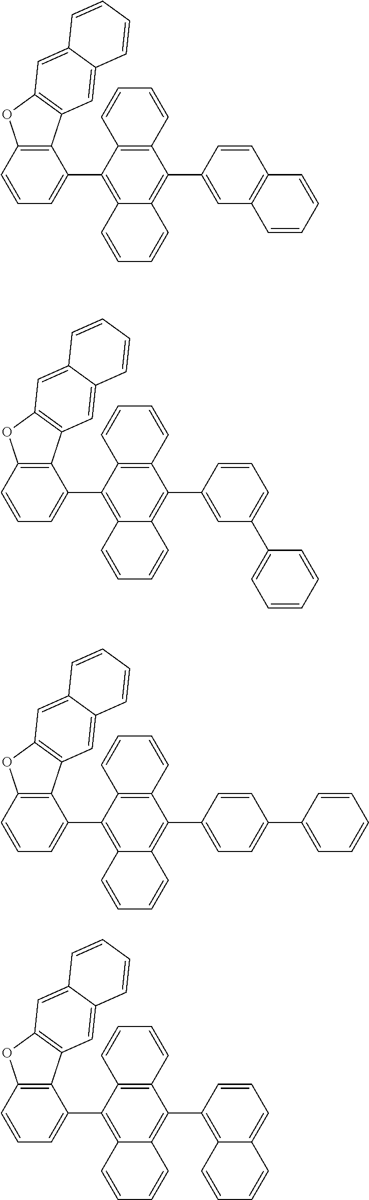

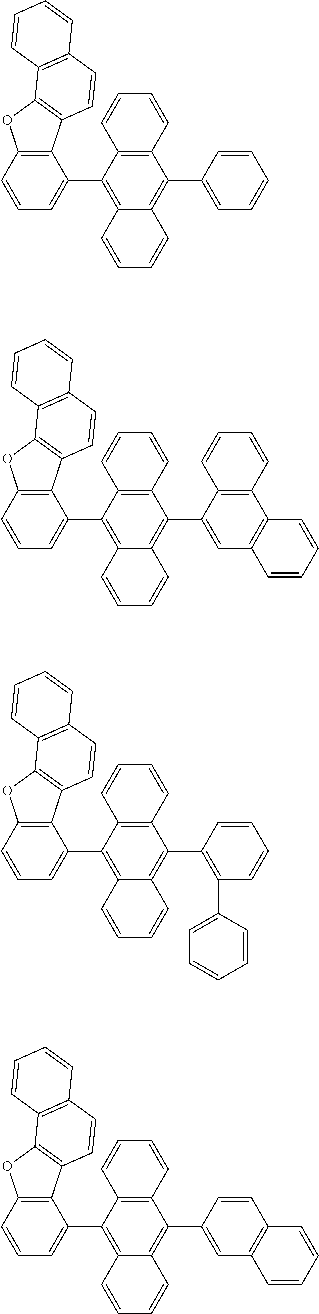 [SMILES: c1ccc(-c2c3ccccc3c(-c3cccc4oc5c6ccccc6ccc5c34)c3ccccc23)cc1 ; c1ccc2c(c1)cc(-c1c3ccccc3c(-c3cccc4oc5c6ccccc6ccc5c34)c3ccccc13)c1ccccc12 ; c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1]

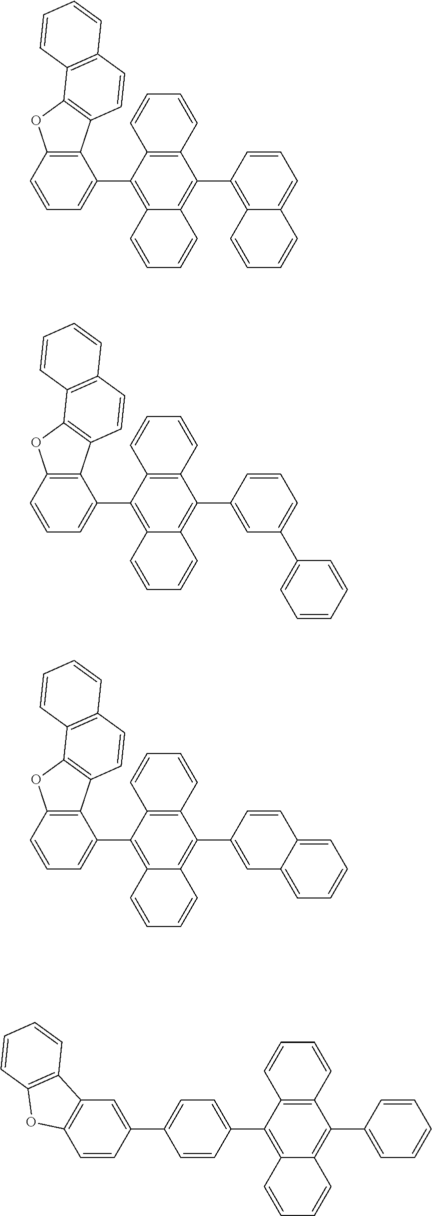 [SMILES: c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc2cc(-c3c4ccccc4c(-c4cccc5oc6c7ccccc7ccc6c45)c4ccccc34)ccc2c1 ; c1ccc(-c2c3ccccc3c(-c3ccc(-c4ccc5oc6ccccc6c5c4)cc3)c3ccccc23)cc1]

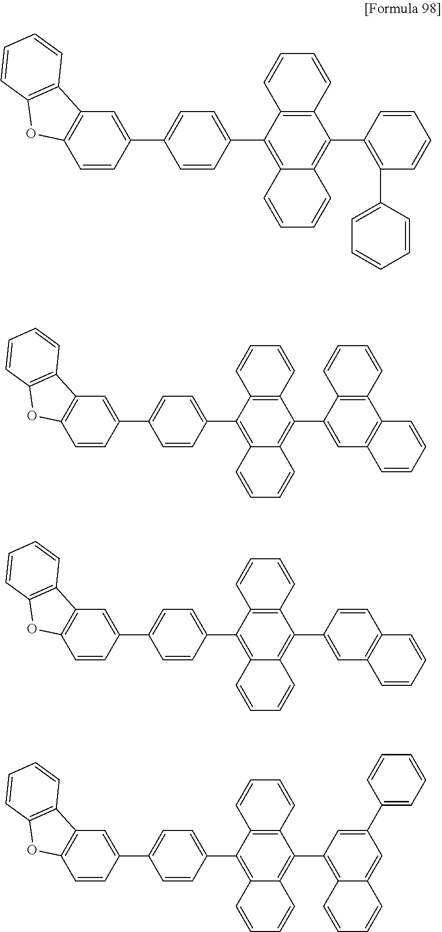 [SMILES: c1ccc(-c2ccccc2-c2c3ccccc3c(-c3ccc(-c4ccc5oc6ccccc6c5c4)cc3)c3ccccc23)cc1 ; c1ccc2c(c1)cc(-c1c3ccccc3c(-c3ccc(-c4ccc5oc6ccccc6c5c4)cc3)c3ccccc13)c1ccccc12 ; c1ccc2cc(-c3c4ccccc4c(-c4ccc(-c5ccc6oc7ccccc7c6c5)cc4)c4ccccc34)ccc2c1 ; c1ccc(-c2cc(-c3c4ccccc4c(-c4ccc(-c5ccc6oc7ccccc7c6c5)cc4)c4ccccc34)c3ccccc3c2)cc1]

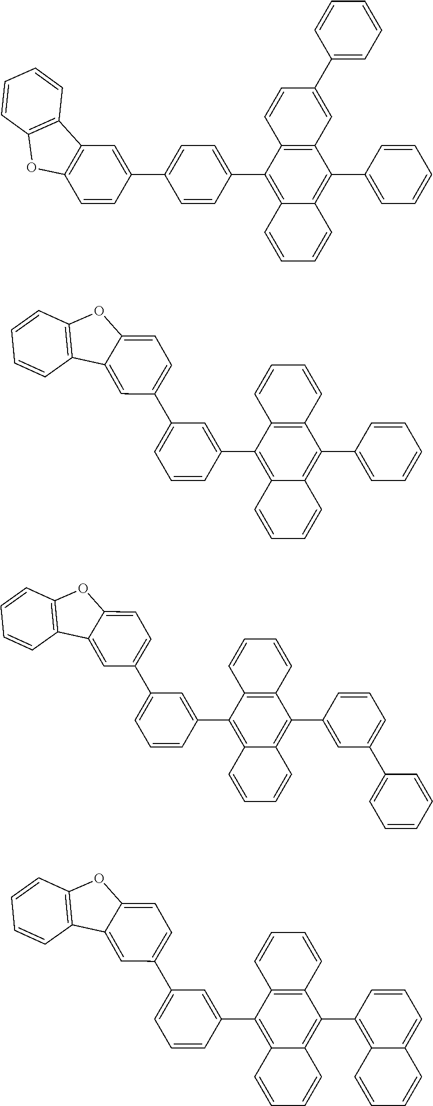 [SMILES: c1ccc(-c2ccc3c(-c4ccc(-c5ccc6oc7ccccc7c6c5)cc4)c4ccccc4c(-c4ccccc4)c3c2)cc1 ; c1ccc(-c2c3ccccc3c(-c3cccc(-c4ccc5oc6ccccc6c5c4)c3)c3ccccc23)cc1 ; c1ccc(-c2cccc(-c3c4ccccc4c(-c4cccc(-c5ccc6oc7ccccc7c6c5)c4)c4ccccc34)c2)cc1 ; c1cc(-c2ccc3oc4ccccc4c3c2)cc(-c2c3ccccc3c(-c3cccc4ccccc34)c3ccccc23)c1]

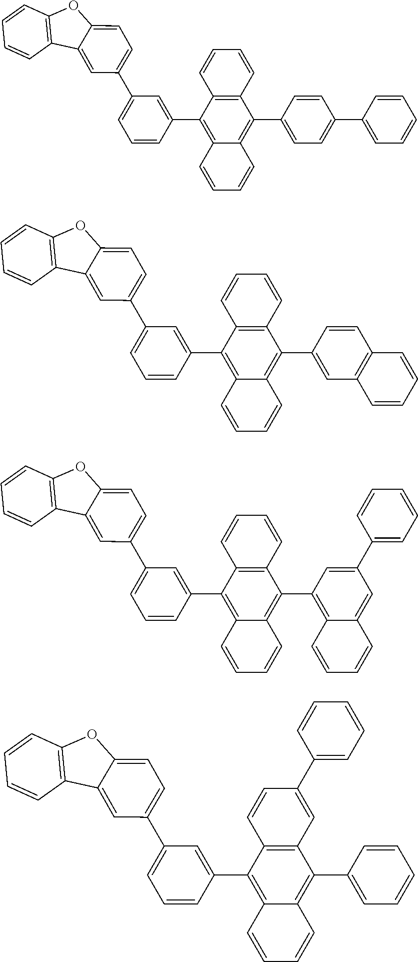

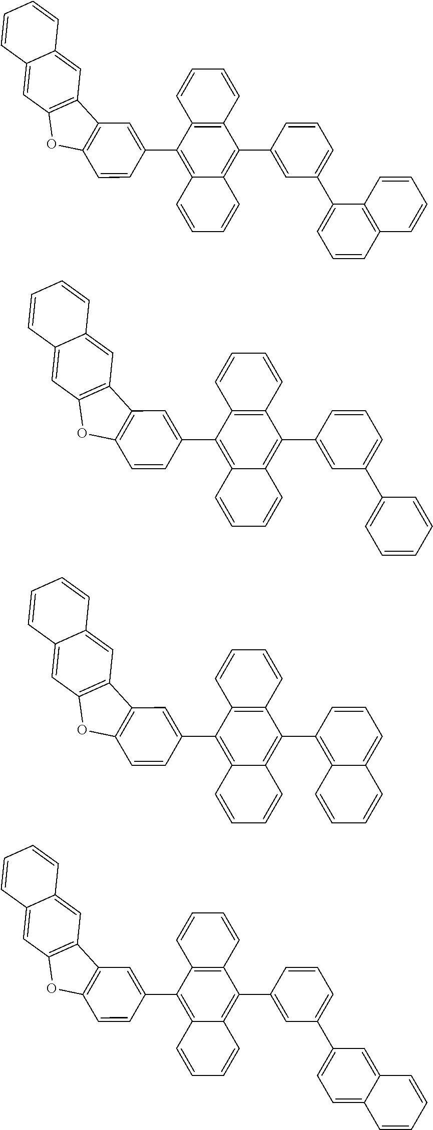 [SMILES: c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1cc(-c2ccc3ccccc3c2)cc(-c2c3ccccc3c(-c3ccc4oc5cc6ccccc6cc5c4c3)c3ccccc23)c1]

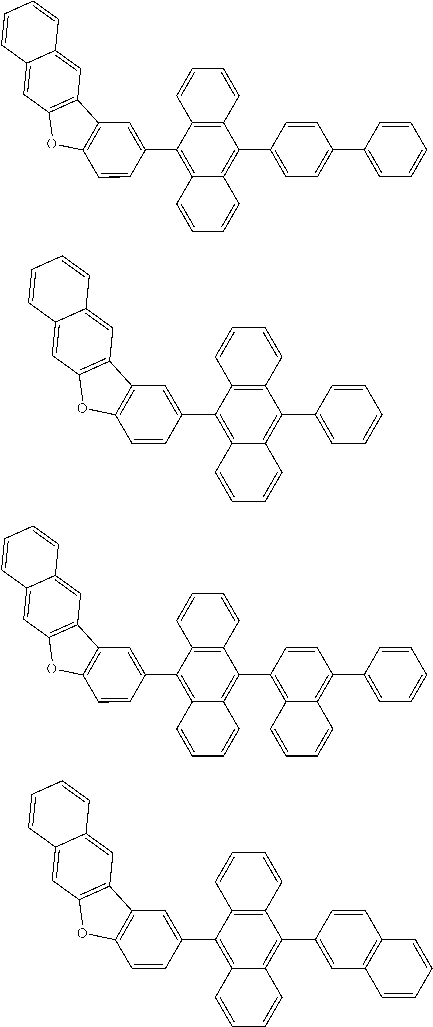 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1]

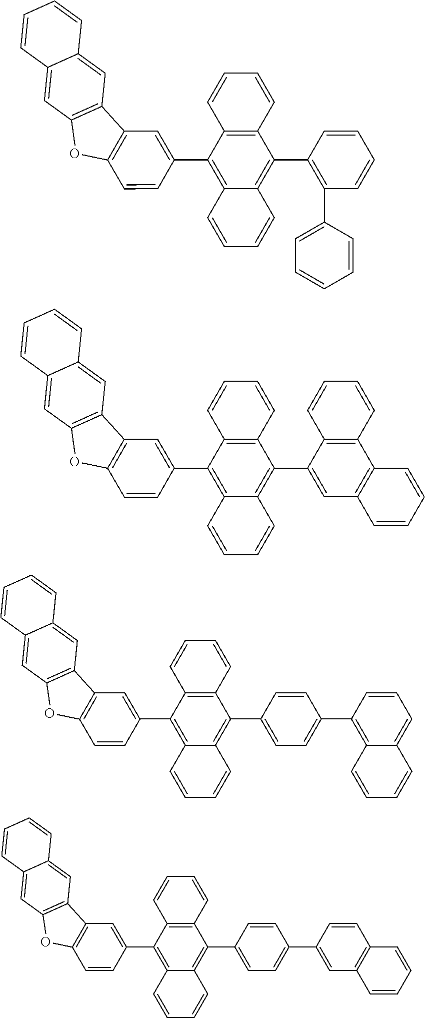 [SMILES: c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1]

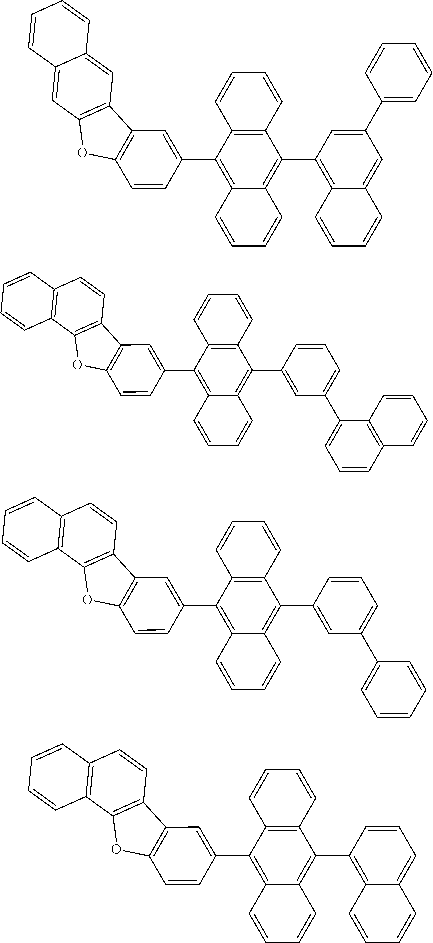 [SMILES: c1ccc(-c2cc(-c3c4ccccc4c(-c4ccc5oc6cc7ccccc7cc6c5c4)c4ccccc34)c3ccccc3c2)cc1 ; c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1]

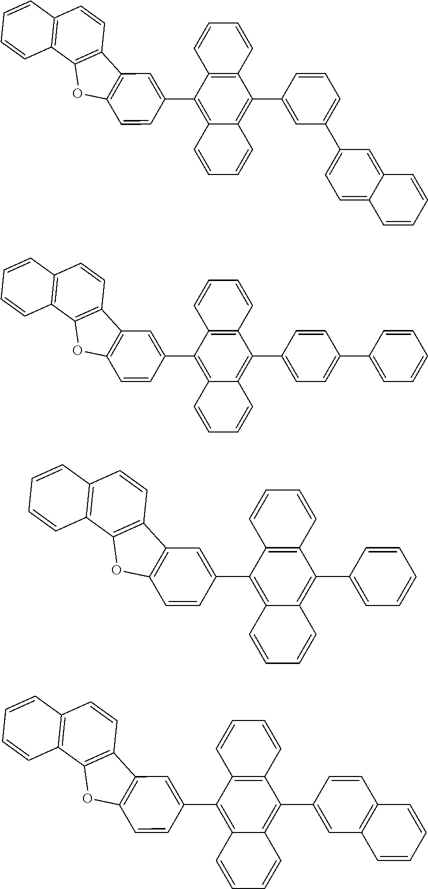 [SMILES: c1cc(-c2ccc3ccccc3c2)cc(-c2c3ccccc3c(-c3ccc4oc5c6ccccc6ccc5c4c3)c3ccccc23)c1 ; c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1]

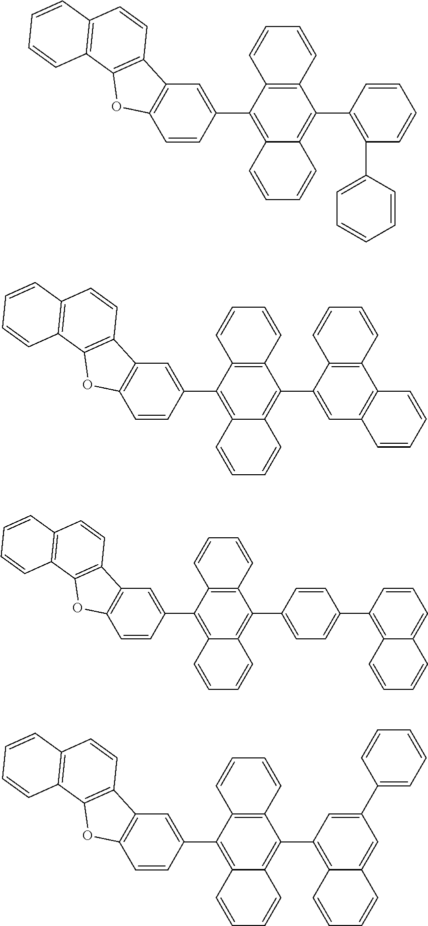

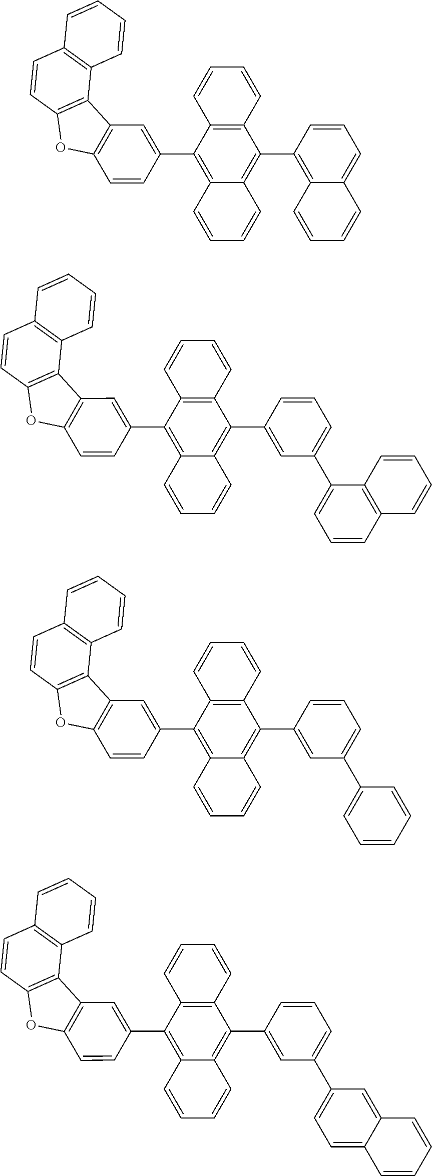 [SMILES: c1ccc2c(-c3c4ccccc4c(-c4ccc5oc6ccc7ccccc7c6c5c4)c4ccccc34)cccc2c1 ; c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

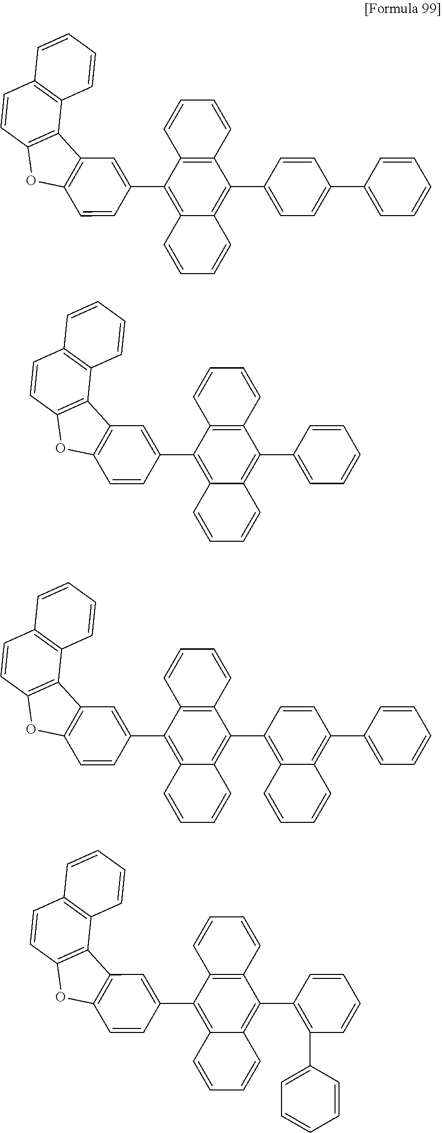 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1]

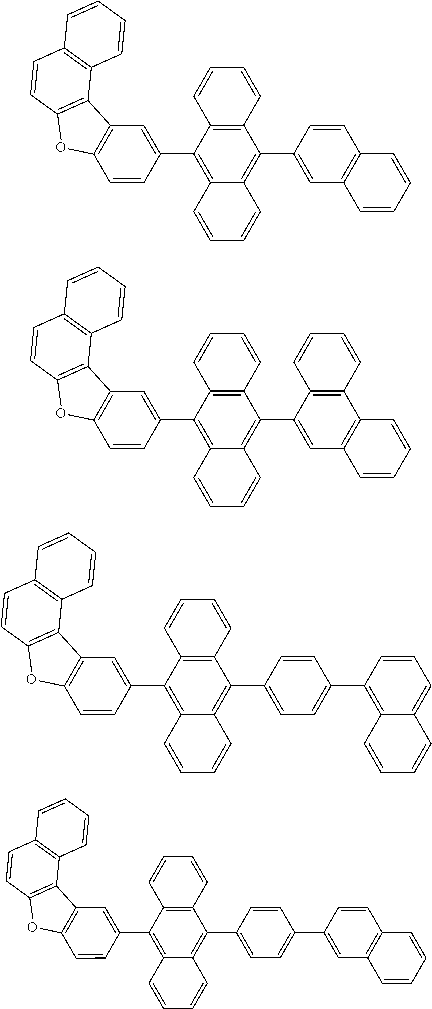 [SMILES: c1ccc2cc(-c3c4ccccc4c(-c4ccc5oc6ccc7ccccc7c6c5c4)c4ccccc34)ccc2c1 ; c1ccc2c(c1)cc(-c1c3ccccc3c(-c3ccc4oc5ccc6ccccc6c5c4c3)c3ccccc13)c1ccccc12 ; c1ccc2c(-c3ccc(-c4c5ccccc5c(-c5ccc6oc7ccc8ccccc8c7c6c5)c5ccccc45)cc3)cccc2c1 ; c1ccc2cc(-c3ccc(-c4c5ccccc5c(-c5ccc6oc7ccc8ccccc8c7c6c5)c5ccccc45)cc3)ccc2c1]

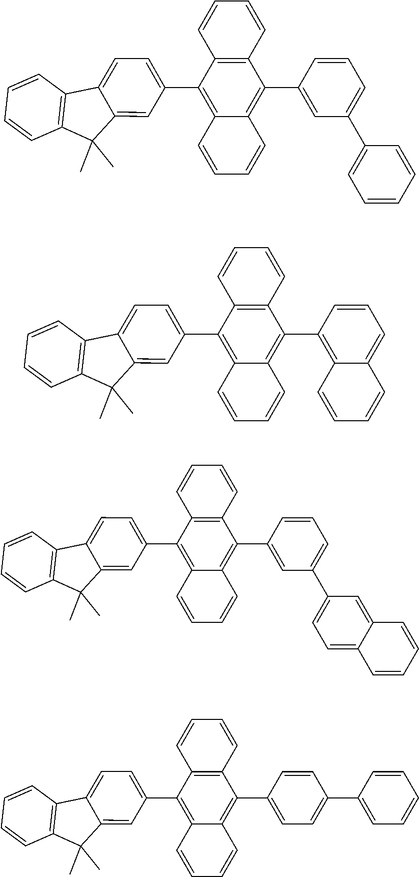

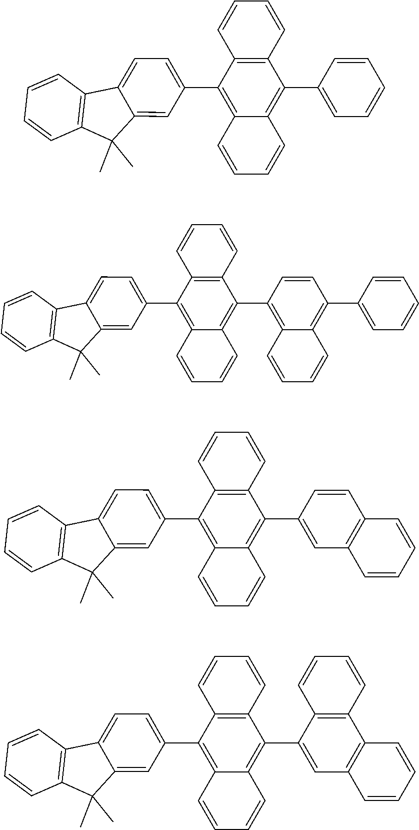

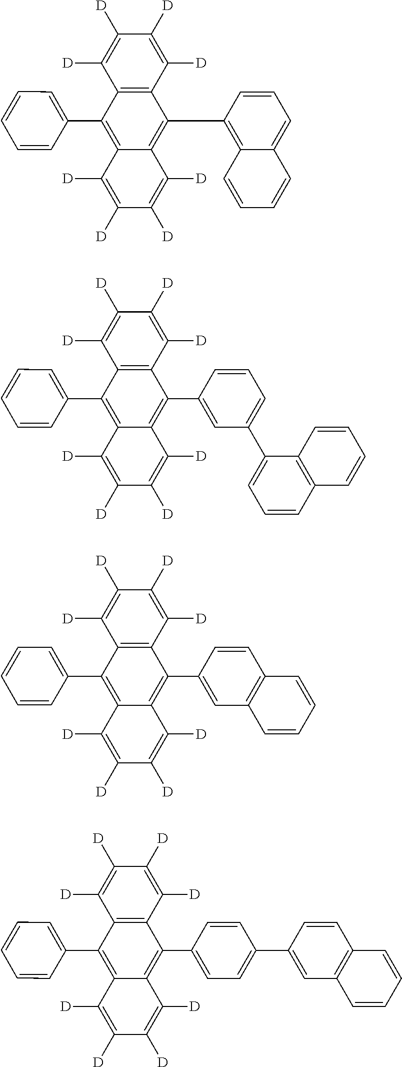 [SMILES: [2H]c1c([2H])c([2H])c2c(-c3cccc4ccccc34)c3c([2H])c([2H])c([2H])c([2H])c3c(-c3ccccc3)c2c1[2H] ; [2H]c1c([2H])c([2H])c2c(-c3cccc(-c4cccc5ccccc45)c3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3ccc(-c4ccc5ccccc5c4)cc3)c3c([2H])c([2H])c([2H])c([2H])c3c(-c3ccccc3)c2c1[2H]]

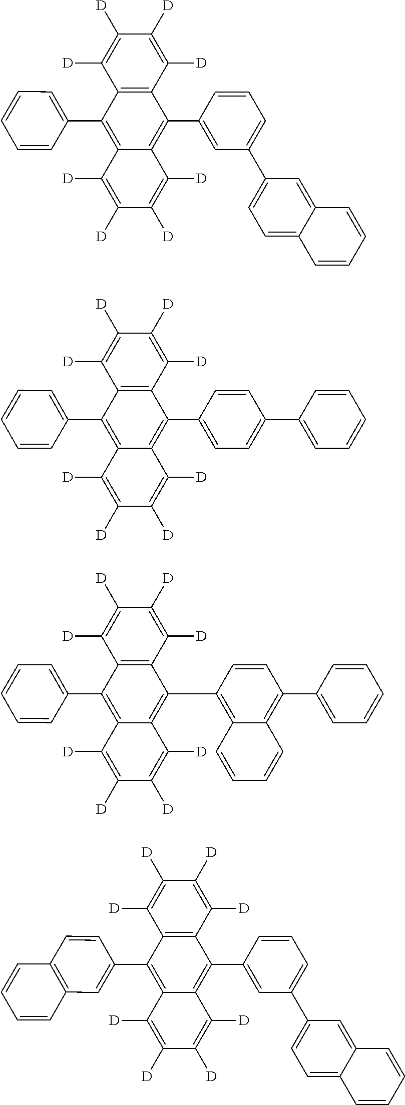

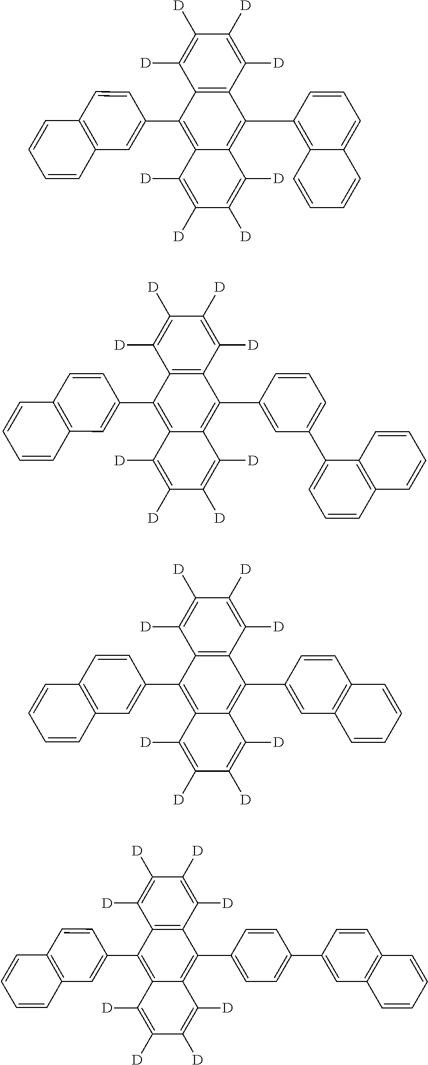 [SMILES: [2H]c1c([2H])c([2H])c2c(-c3cccc4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4ccccc4c3)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccc(-c4ccc5ccccc5c4)cc3)c2c1[2H]]

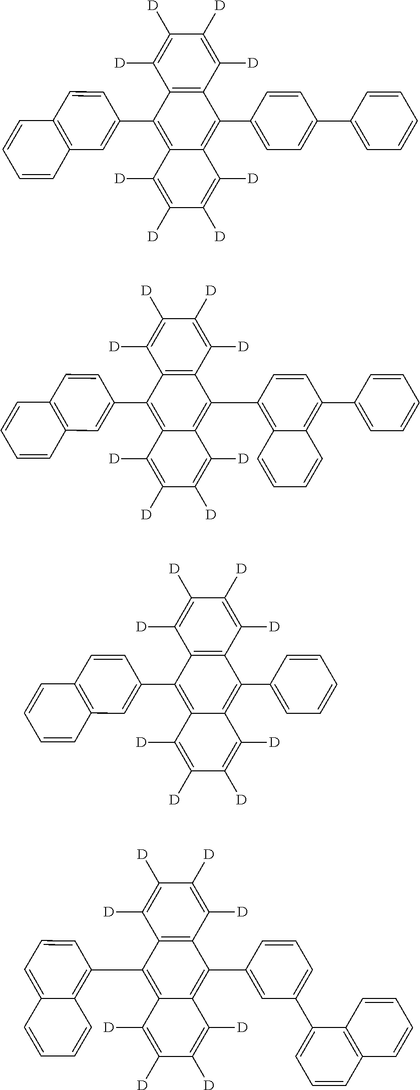 [SMILES: [2H]c1c([2H])c([2H])c2c(-c3ccc4ccccc4c3)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4ccccc4c3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3cccc(-c4cccc5ccccc45)c3)c2c1[2H]]

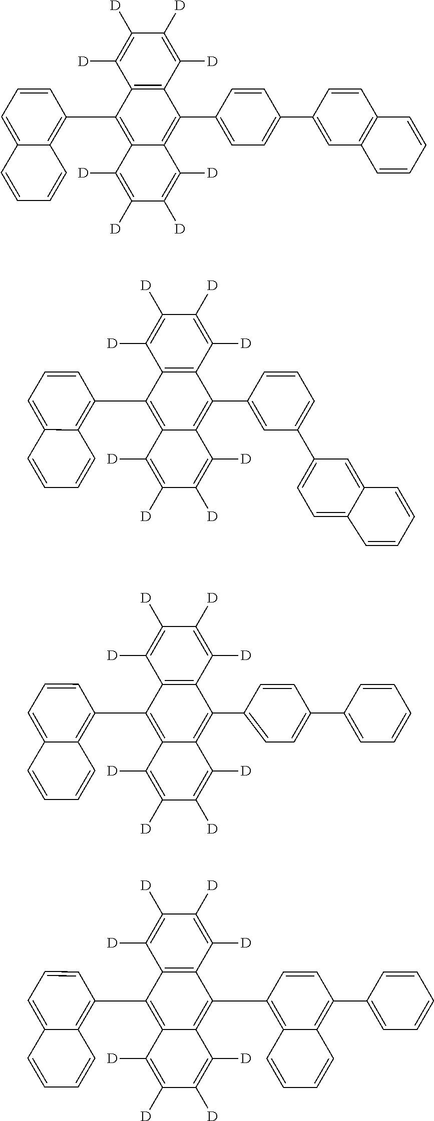 [SMILES: [2H]c1c([2H])c([2H])c2c(-c3cccc4ccccc34)c3c([2H])c([2H])c([2H])c([2H])c3c(-c3ccc(-c4ccc5ccccc5c4)cc3)c2c1[2H] ; [2H]c1c([2H])c([2H])c2c(-c3cccc4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3ccc(-c4ccccc4)cc3)c2c1[2H] ; [2H]c1c([2H])c([2H])c2c(-c3ccc(-c4ccccc4)c4ccccc34)c3c([2H])c([2H])c([2H])c([2H])c3c(-c3cccc4ccccc34)c2c1[2H]]

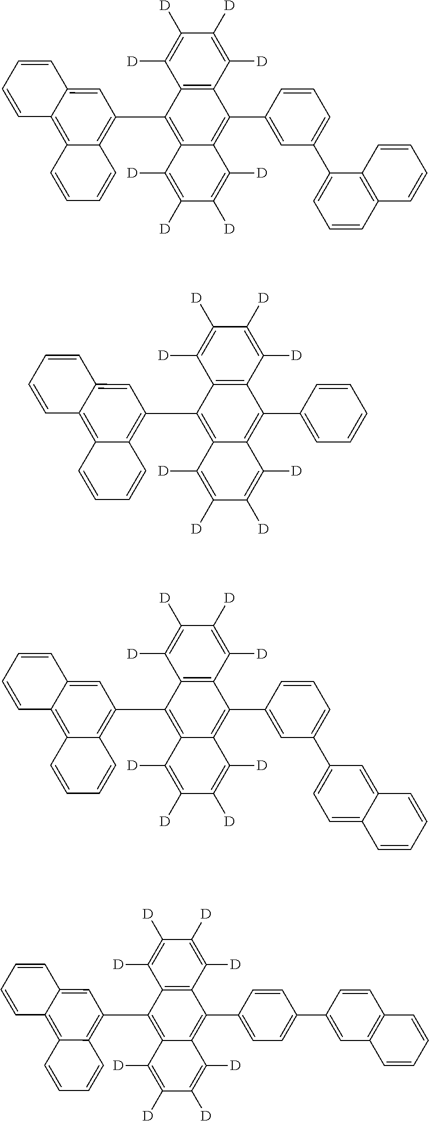 [SMILES: [2H]c1c([2H])c([2H])c2c(-c3cc4ccccc4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cc4ccccc4c4ccccc34)c3c([2H])c([2H])c([2H])c([2H])c3c(-c3ccccc3)c2c1[2H] ; [2H]c1c([2H])c([2H])c2c(-c3cc4ccccc4c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cc4ccccc4c4ccccc34)c3c([2H])c([2H])c([2H])c([2H])c3c(-c3ccc(-c4ccc5ccccc5c4)cc3)c2c1[2H]]

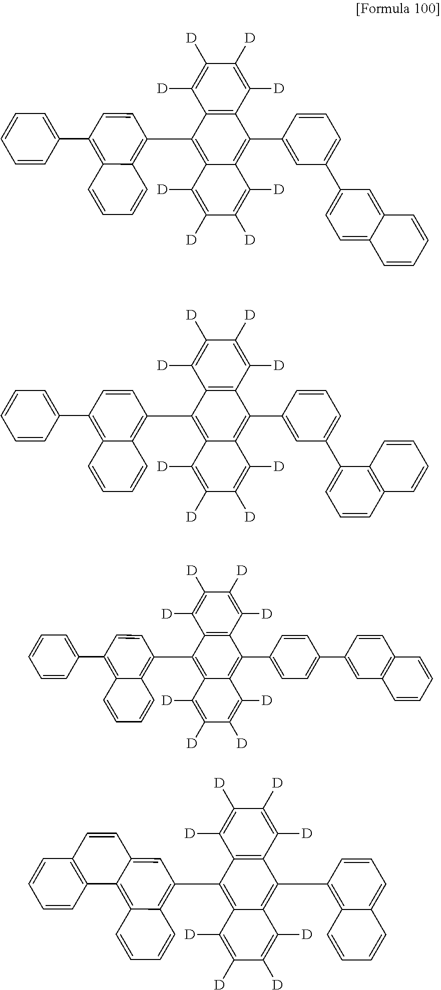 [SMILES: [2H]c1c([2H])c([2H])c2c(-c3ccc(-c4ccccc4)c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3ccc(-c4ccccc4)c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3ccc(-c4ccccc4)c4ccccc34)c3c([2H])c([2H])c([2H])c([2H])c3c(-c3ccc(-c4ccc5ccccc5c4)cc3)c2c1[2H] ; [2H]c1c([2H])c([2H])c2c(-c3cc4ccc5ccccc5c4c4ccccc34)c3c([2H])c([2H])c([2H])c([2H])c3c(-c3cccc4ccccc34)c2c1[2H]]

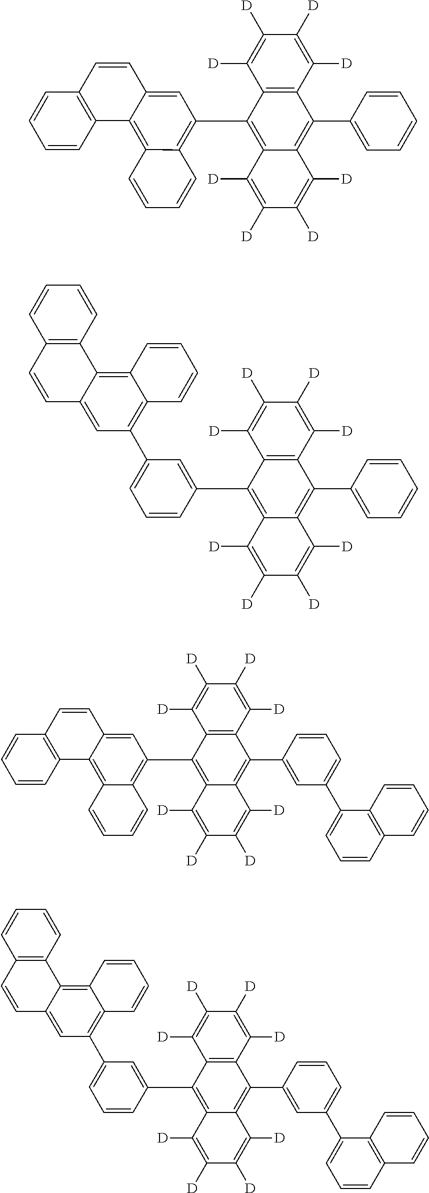 [SMILES: [2H]c1c([2H])c([2H])c2c(-c3cc4ccc5ccccc5c4c4ccccc34)c3c([2H])c([2H])c([2H])c([2H])c3c(-c3ccccc3)c2c1[2H] ; [2H]c1c([2H])c([2H])c2c(-c3cccc(-c4cc5ccc6ccccc6c5c5ccccc45)c3)c3c([2H])c([2H])c([2H])c([2H])c3c(-c3ccccc3)c2c1[2H] ; [2H]c1c([2H])c([2H])c2c(-c3cc4ccc5ccccc5c4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cccc(-c4cc5ccc6ccccc6c5c5ccccc45)c3)c3c([2H])c([2H])c([2H])c([2H])c3c(-c3cccc(-c4cccc5ccccc45)c3)c2c1[2H]]

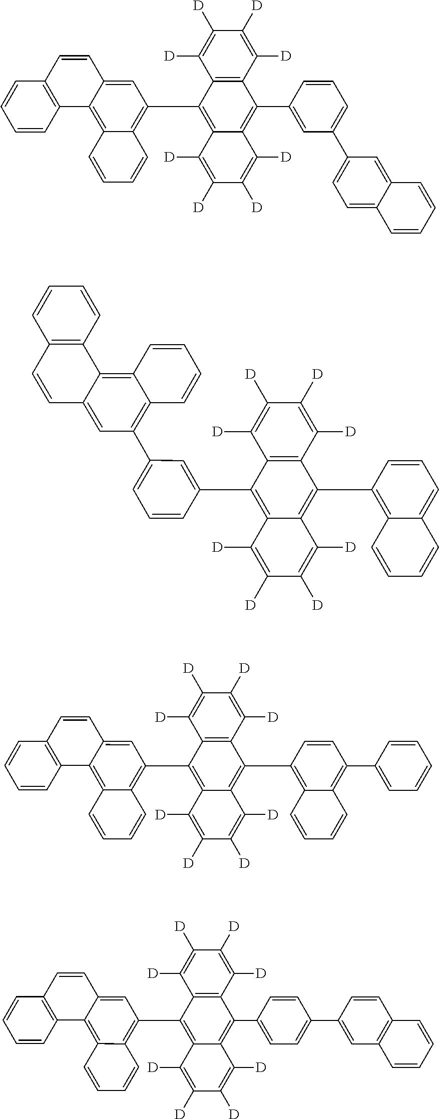 [SMILES: [2H]c1c([2H])c([2H])c2c(-c3cc4ccc5ccccc5c4c4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3cccc(-c4cc5ccc6ccccc6c5c5ccccc45)c3)c2c1[2H] ; [2H]c1c([2H])c([2H])c2c(-c3cc4ccc5ccccc5c4c4ccccc34)c3c([2H])c([2H])c([2H])c([2H])c3c(-c3ccc(-c4ccccc4)c4ccccc34)c2c1[2H] ; [2H]c1c([2H])c([2H])c2c(-c3cc4ccc5ccccc5c4c4ccccc34)c3c([2H])c([2H])c([2H])c([2H])c3c(-c3ccc(-c4ccc5ccccc5c4)cc3)c2c1[2H]]

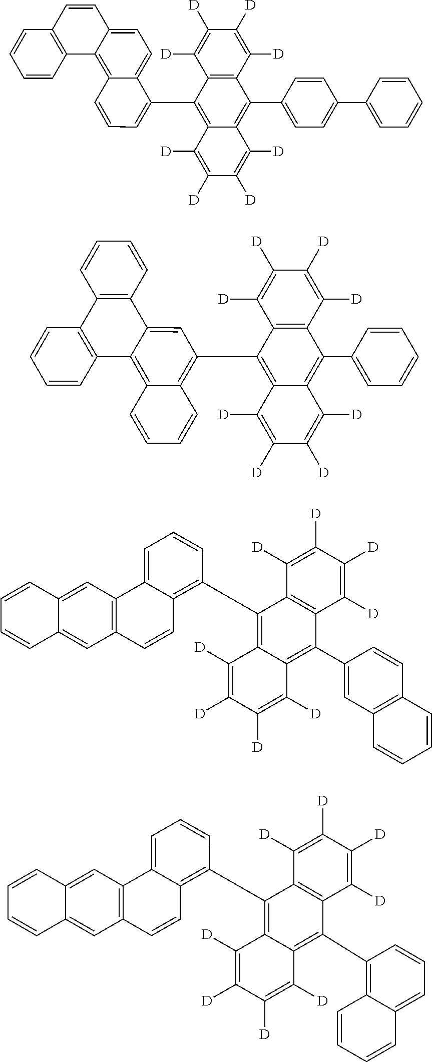

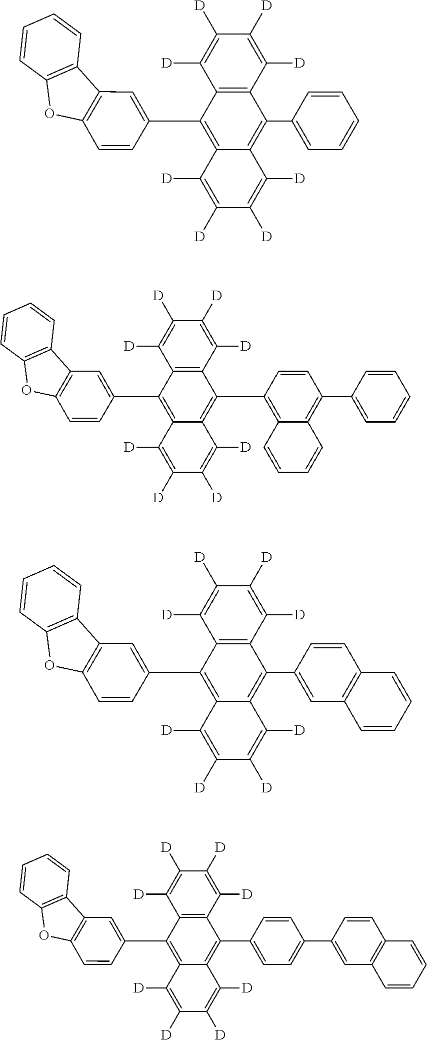 [SMILES: [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc4ccccc4c3)c2c1[2H] ; [2H]c1c([2H])c([2H])c2c(-c3ccc4oc5ccccc5c4c3)c3c([2H])c([2H])c([2H])c([2H])c3c(-c3ccc(-c4ccc5ccccc5c4)cc3)c2c1[2H]]

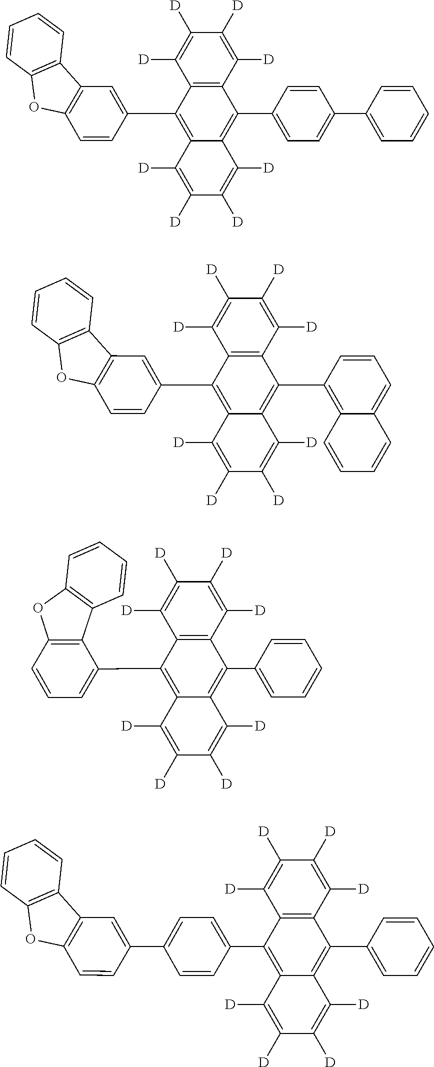 [SMILES: [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccccc5c4c3)c2c1[2H] ; [2H]c1c([2H])c([2H])c2c(-c3cccc4oc5ccccc5c34)c3c([2H])c([2H])c([2H])c([2H])c3c(-c3ccccc3)c2c1[2H] ; [2H]c1c([2H])c([2H])c2c(-c3ccc(-c4ccc5oc6ccccc6c5c4)cc3)c3c([2H])c([2H])c([2H])c([2H])c3c(-c3ccccc3)c2c1[2H]]

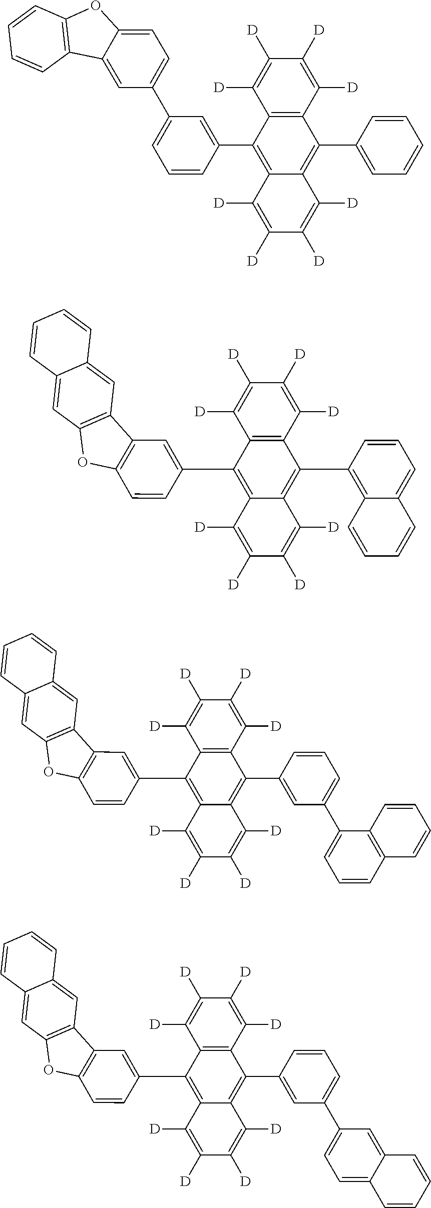 [SMILES: [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4ccc5ccccc5c4)c3)c2c1[2H]]

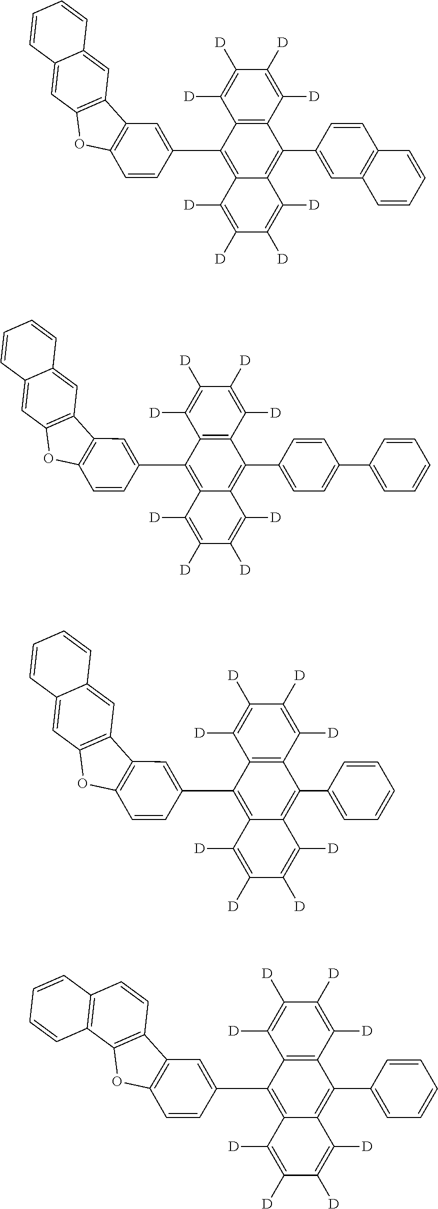 [SMILES: [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H] ; [2H]c1c([2H])c([2H])c2c(-c3ccc4oc5c6ccccc6ccc5c4c3)c3c([2H])c([2H])c([2H])c([2H])c3c(-c3ccccc3)c2c1[2H]]

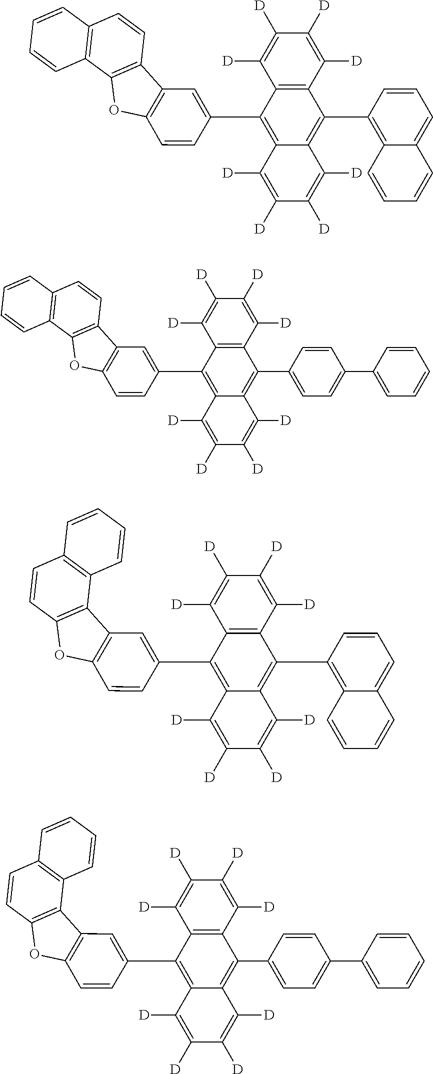 [SMILES: [2H]c1c([2H])c([2H])c2c(-c3cccc4ccccc34)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccc6ccccc6c5c4c3)c2c1[2H] ; [2H]c1c([2H])c([2H])c2c(-c3ccc4oc5ccc6ccccc6c5c4c3)c3c([2H])c([2H])c([2H])c([2H])c3c(-c3ccc(-c4ccccc4)cc3)c2c1[2H]]

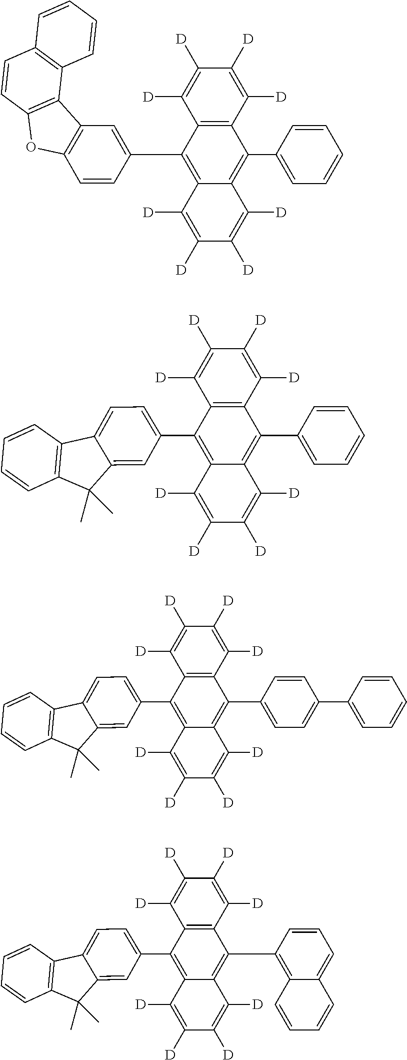 [SMILES: [2H]c1c([2H])c([2H])c2c(-c3ccc4oc5ccc6ccccc6c5c4c3)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccccc3)c2c1[2H] ; [2H]c1c([2H])c([2H])c2c(-c3ccc4c(c3)C(C)(C)c3ccccc3-4)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4c(c3)C(C)(C)c3ccccc3-4)c2c1[2H]]

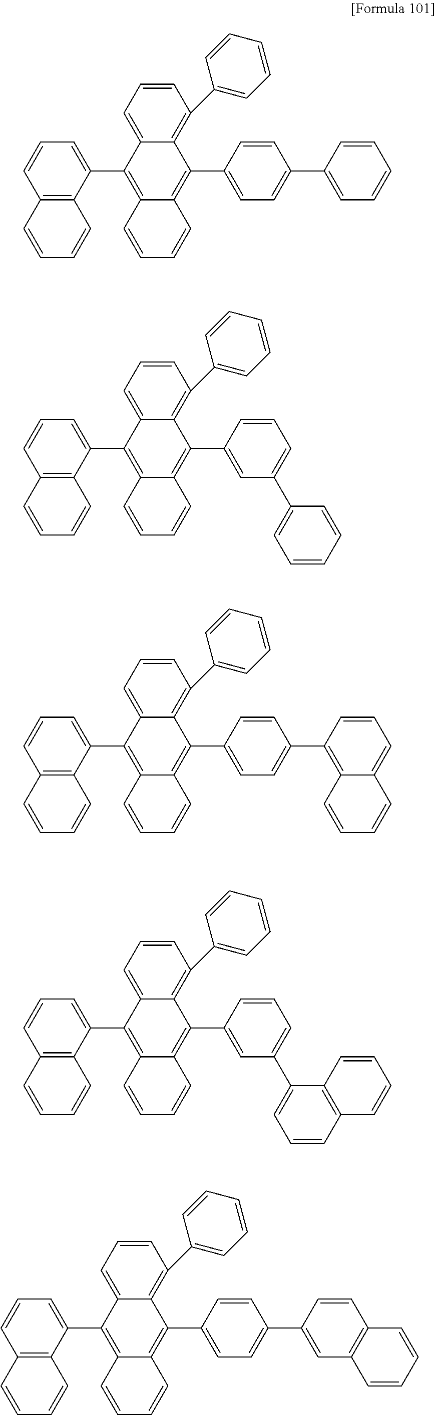

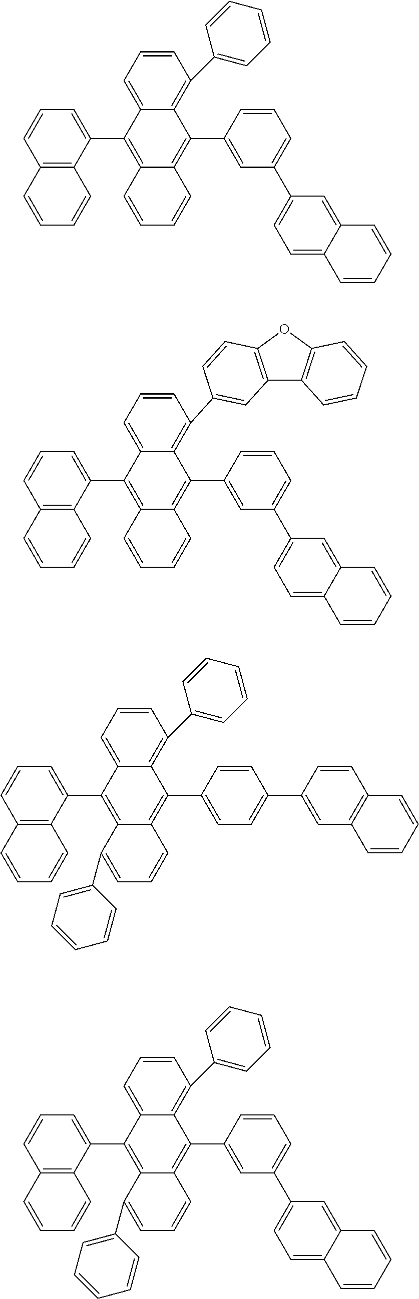

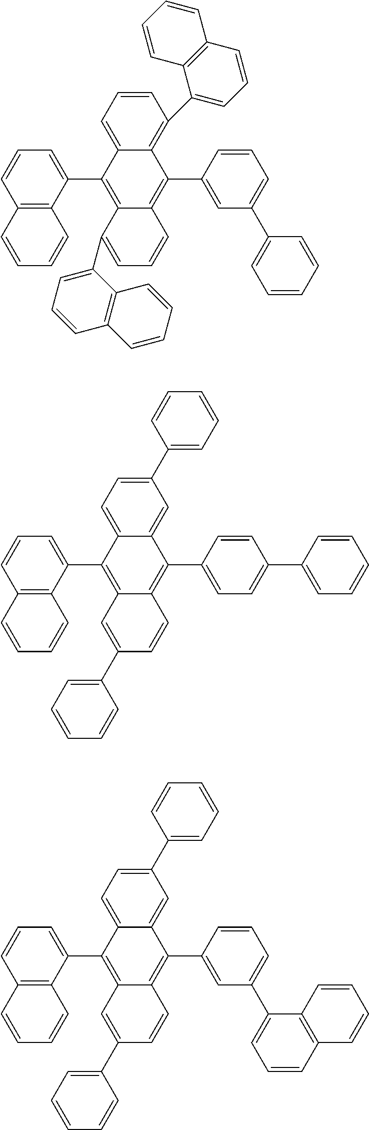

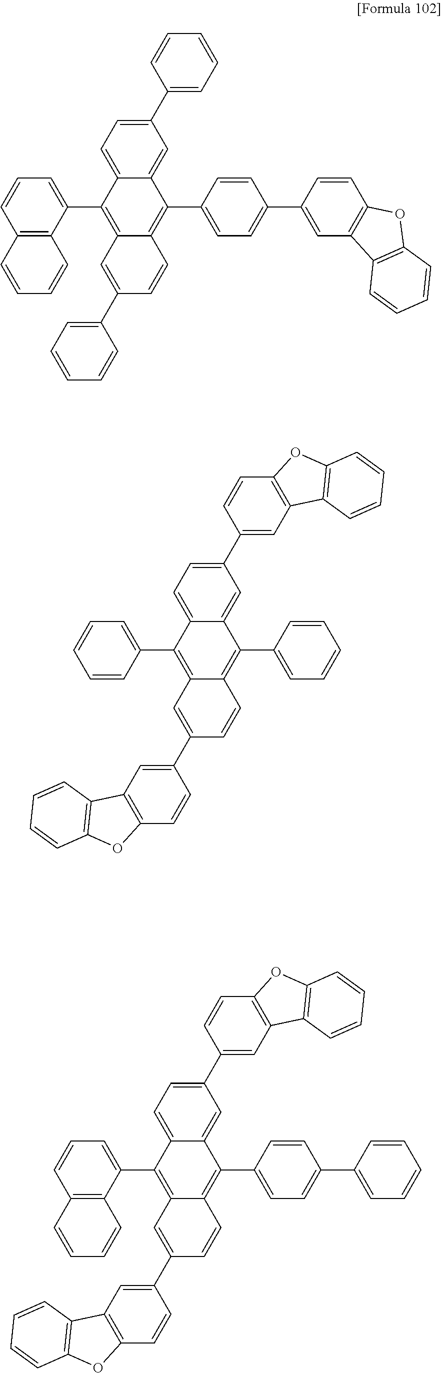 [SMILES: c1ccc(-c2ccc3c(-c4cccc5ccccc45)c4cc(-c5ccccc5)ccc4c(-c4ccc(-c5ccc6oc7ccccc7c6c5)cc4)c3c2)cc1 ; c1ccc(-c2c3ccc(-c4ccc5oc6ccccc6c5c4)cc3c(-c3ccccc3)c3ccc(-c4ccc5oc6ccccc6c5c4)cc23)cc1 ; c1ccc(-c2ccc(-c3c4cc(-c5ccc6oc7ccccc7c6c5)ccc4c(-c4cccc5ccccc45)c4cc(-c5ccc6oc7ccccc7c6c5)ccc34)cc2)cc1]

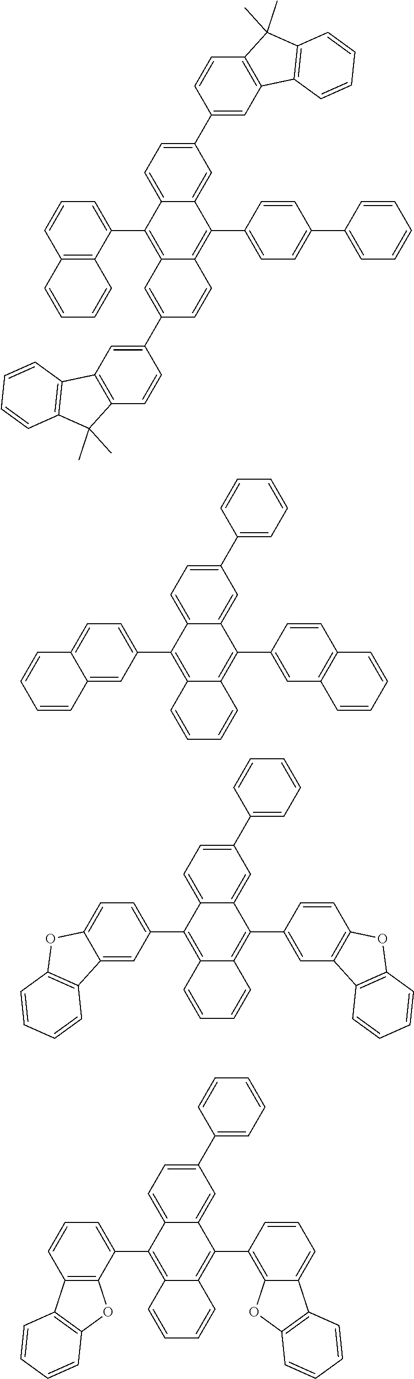

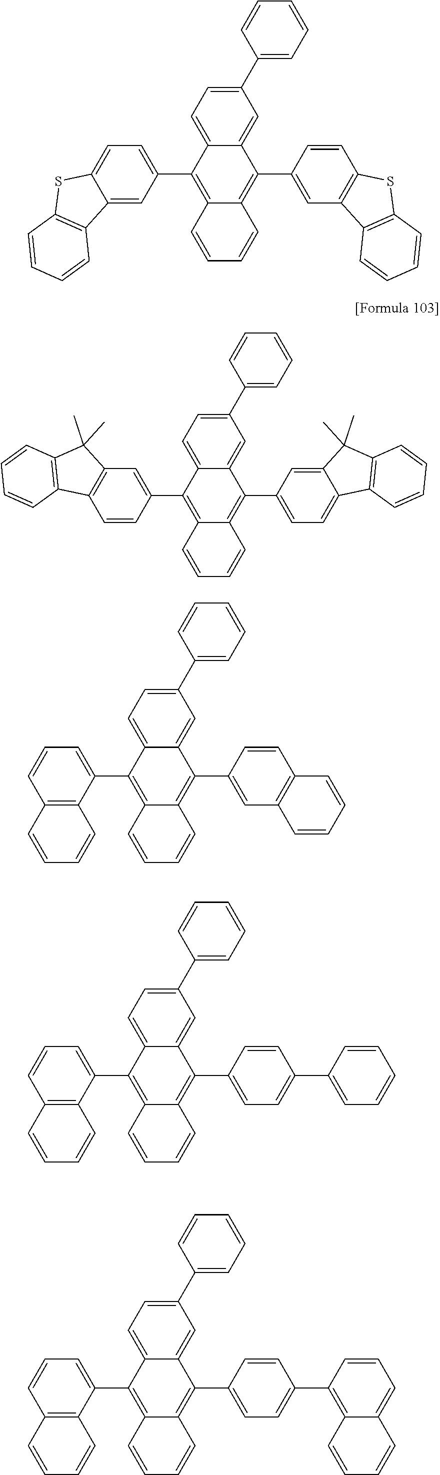

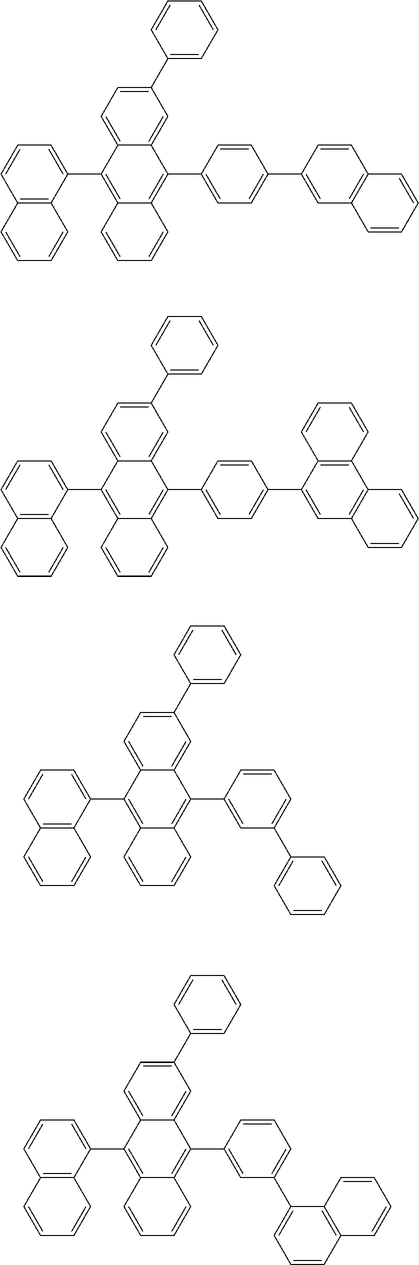

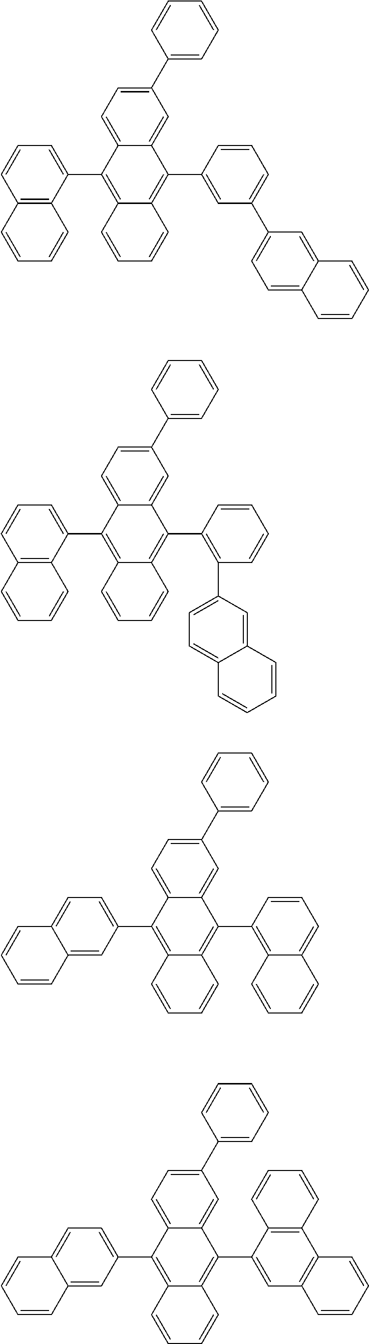

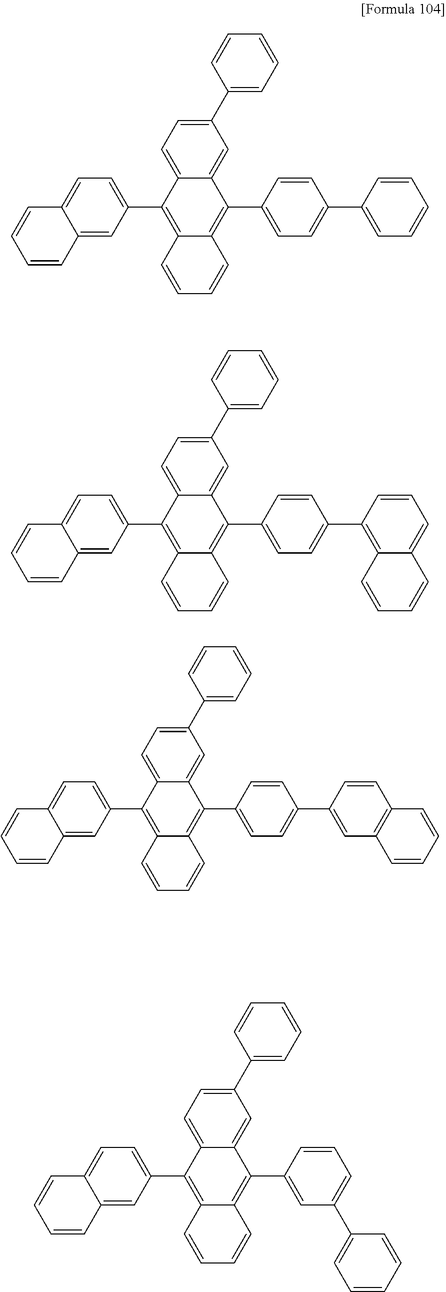

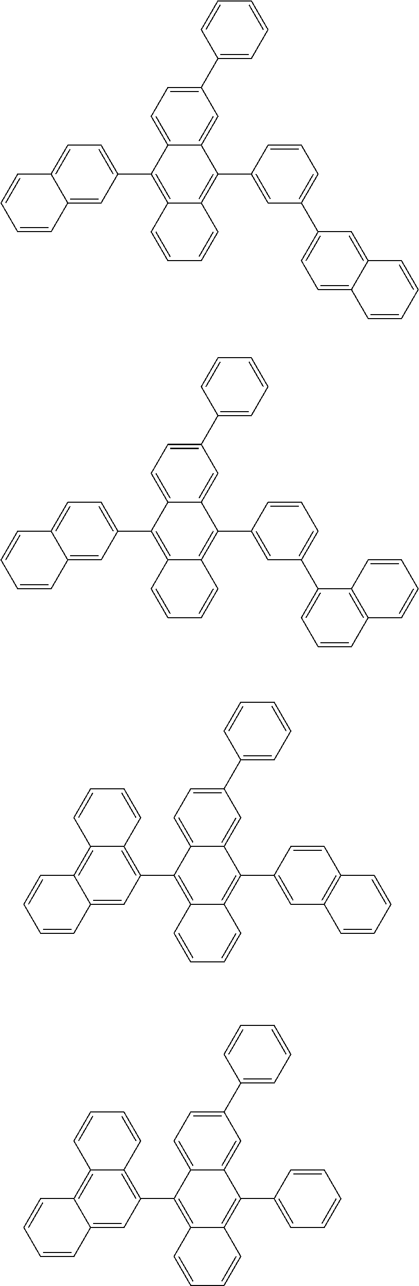

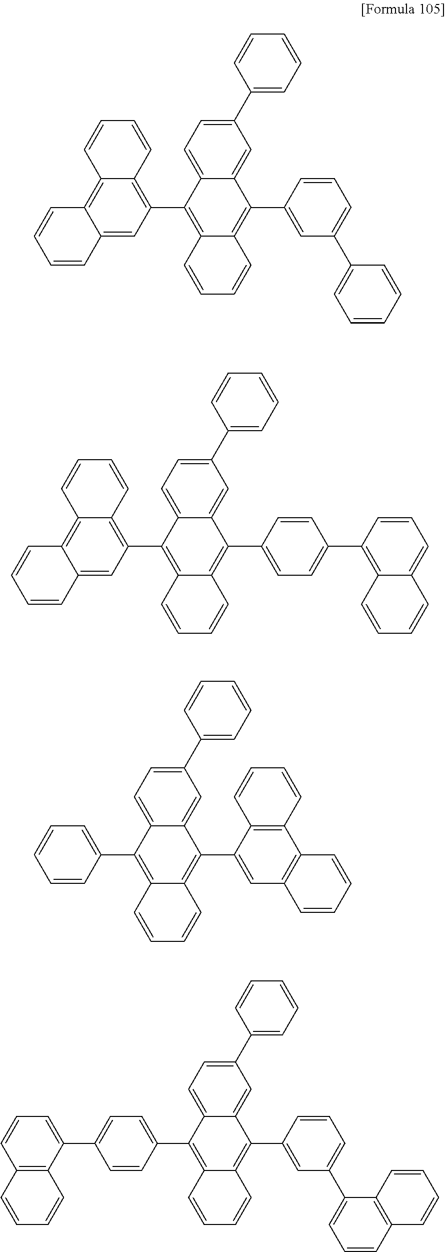

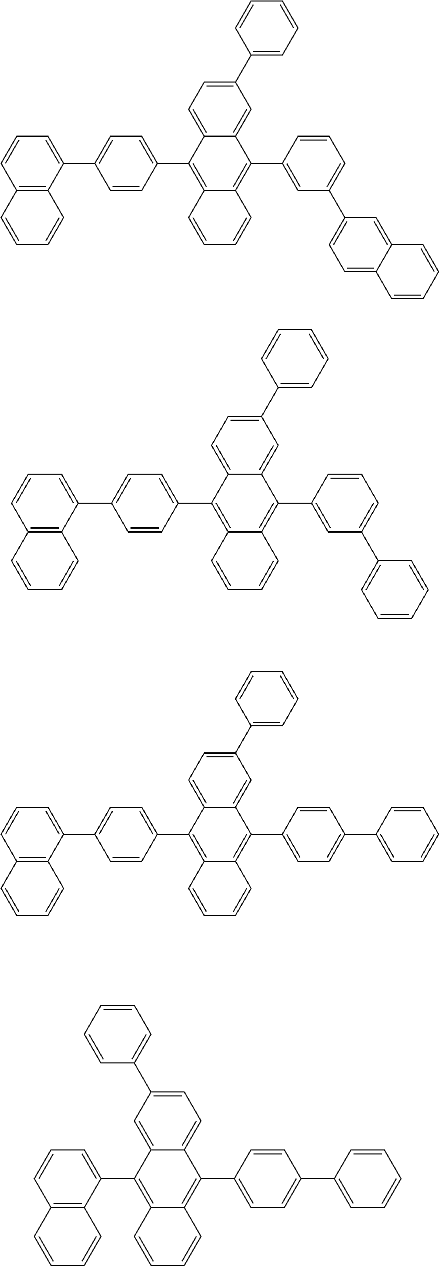

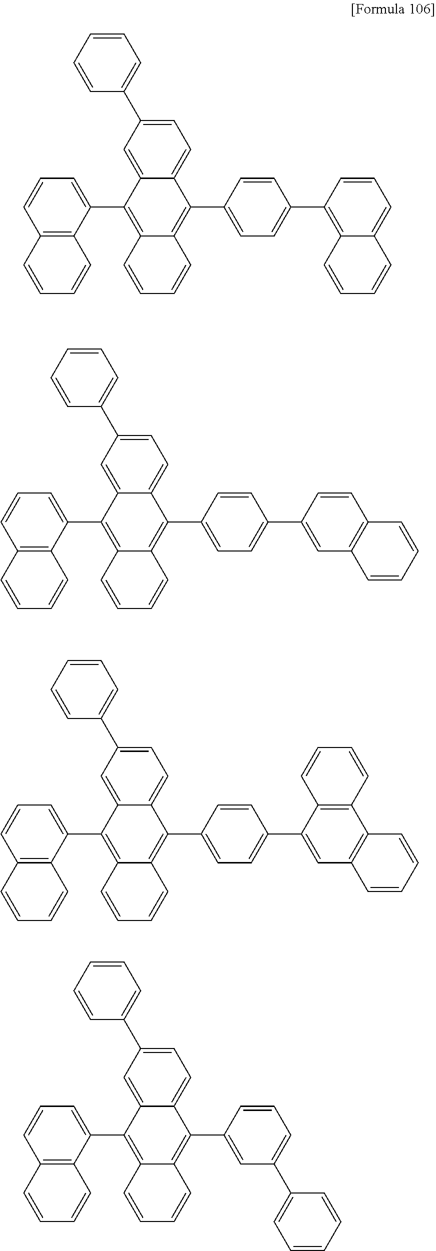

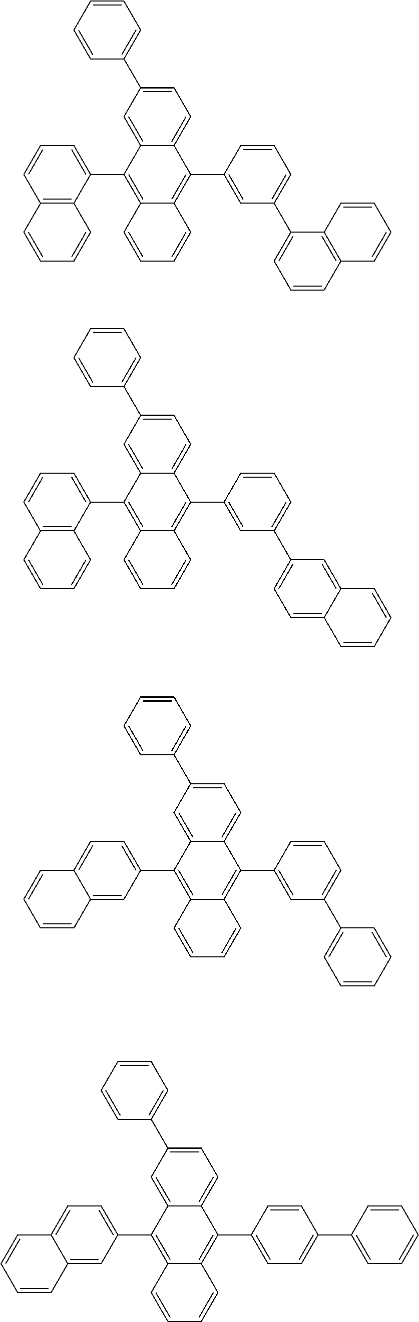

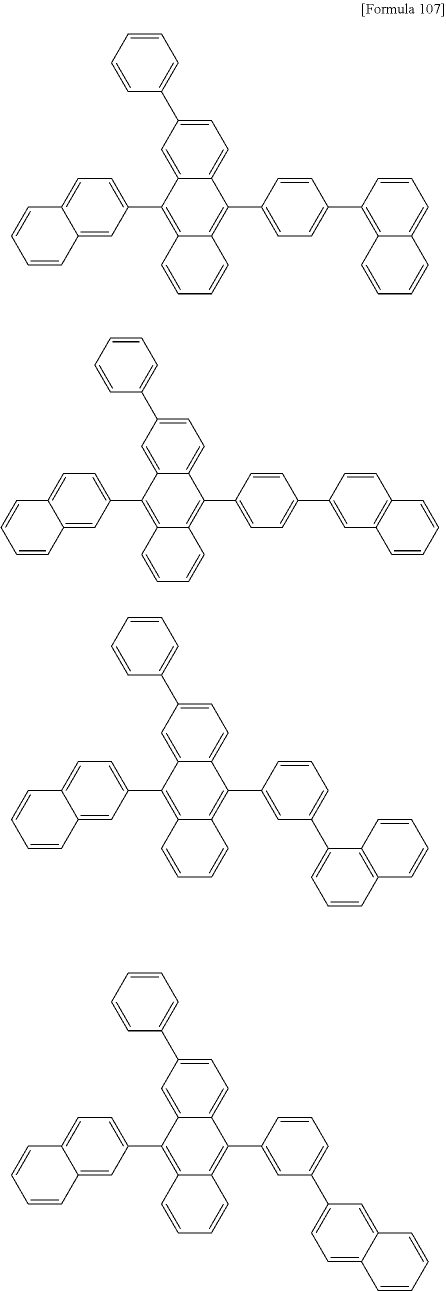

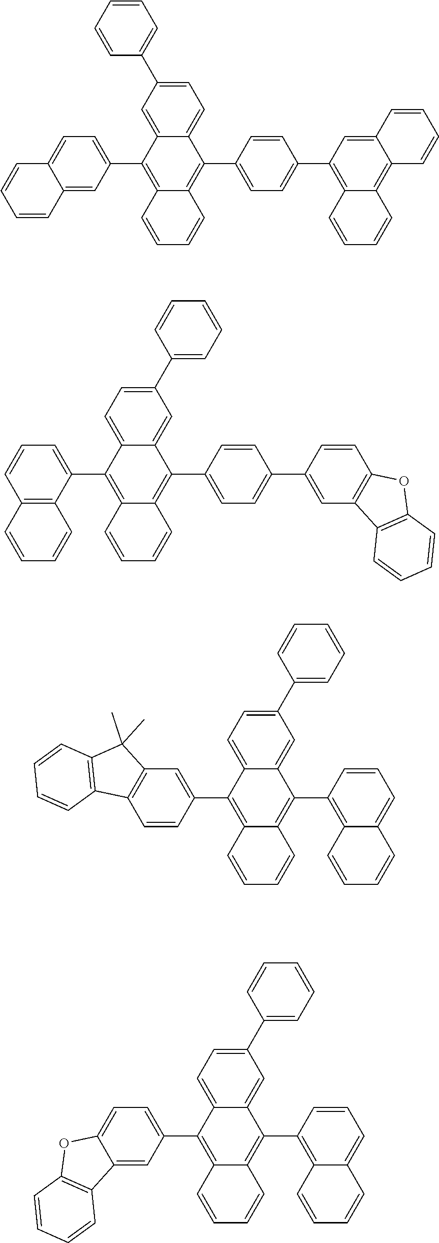

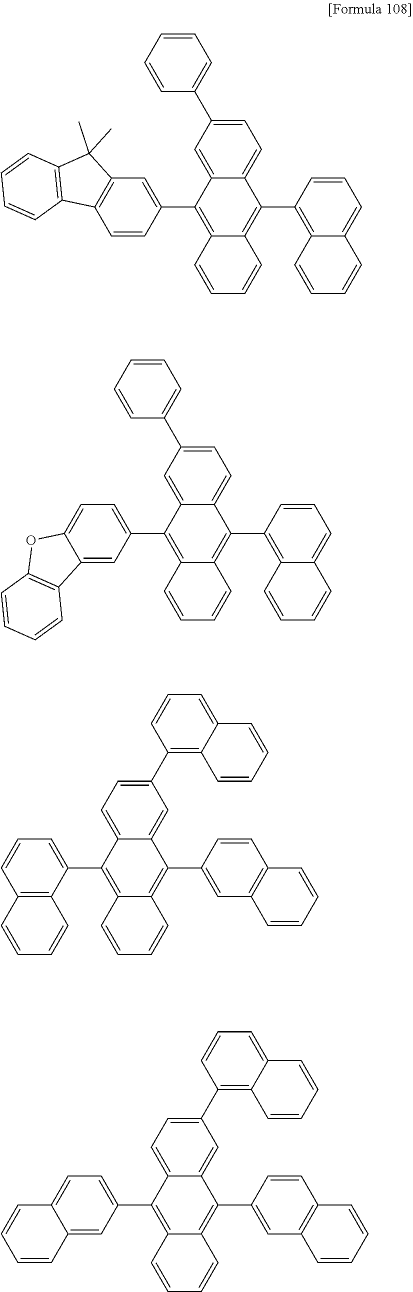

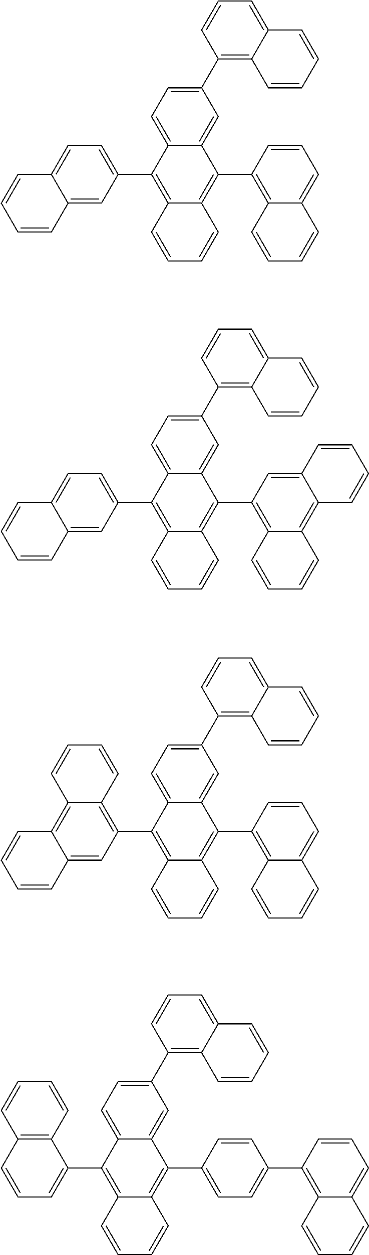

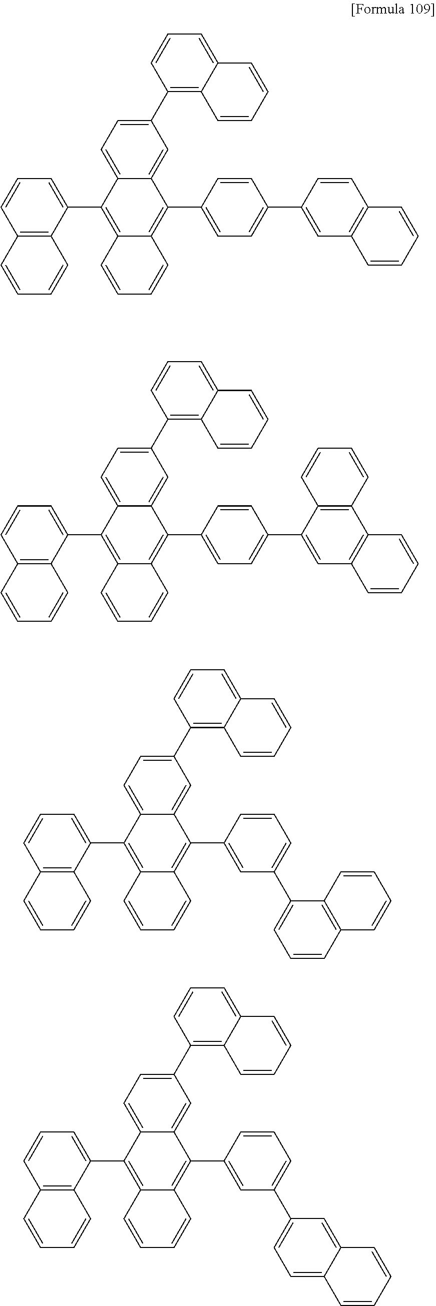

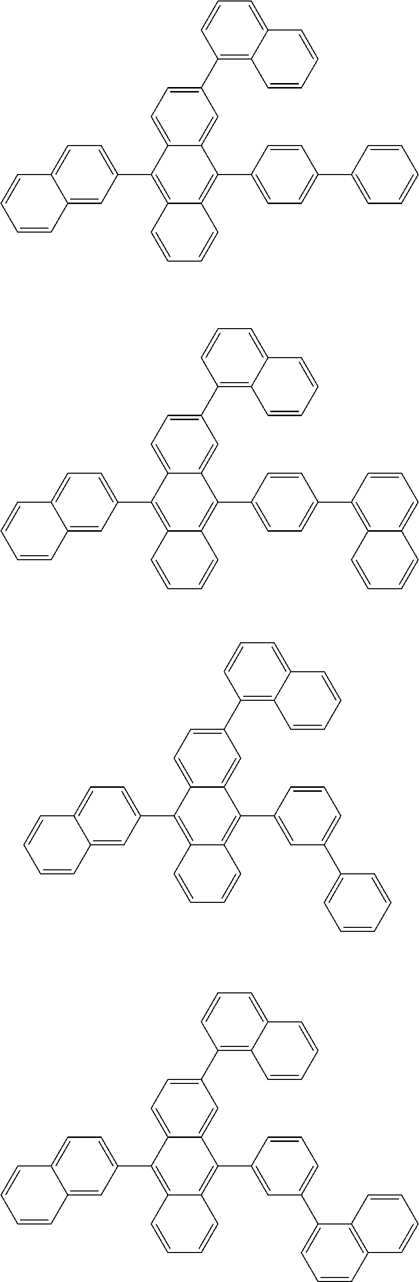

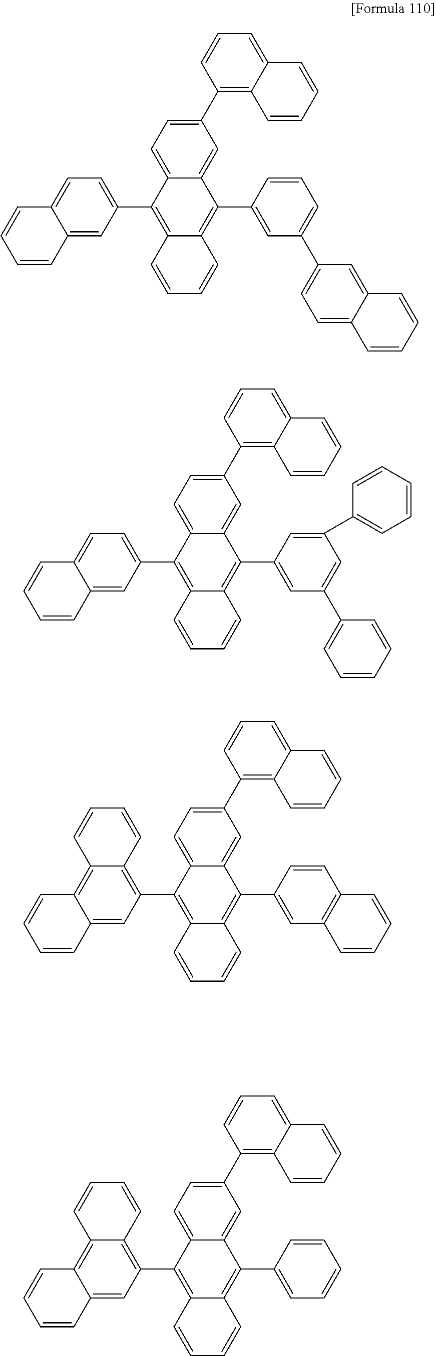

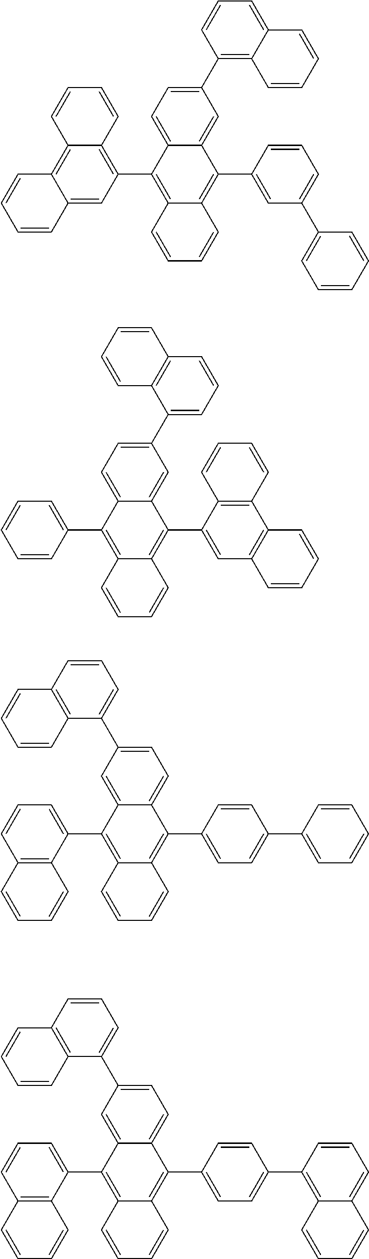

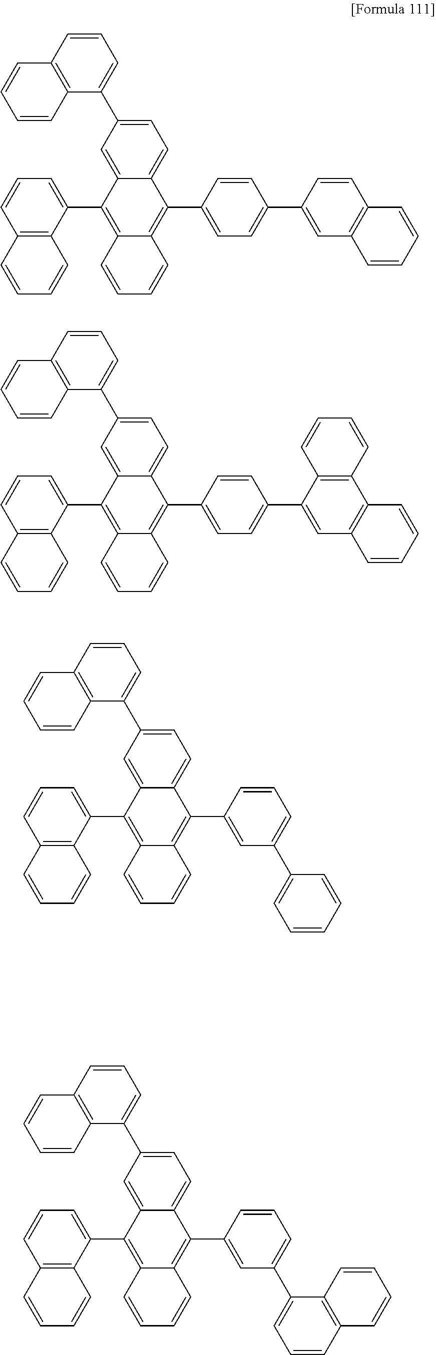

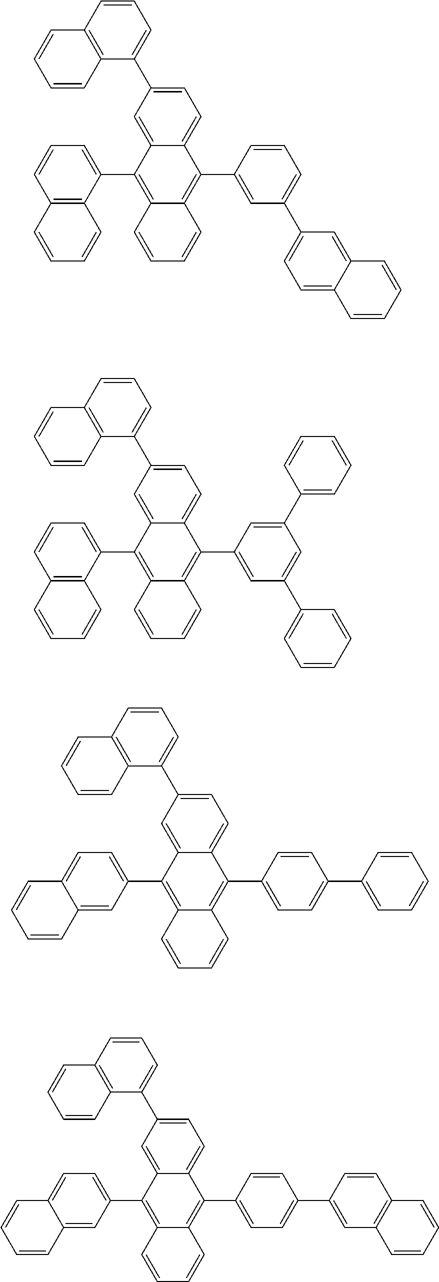

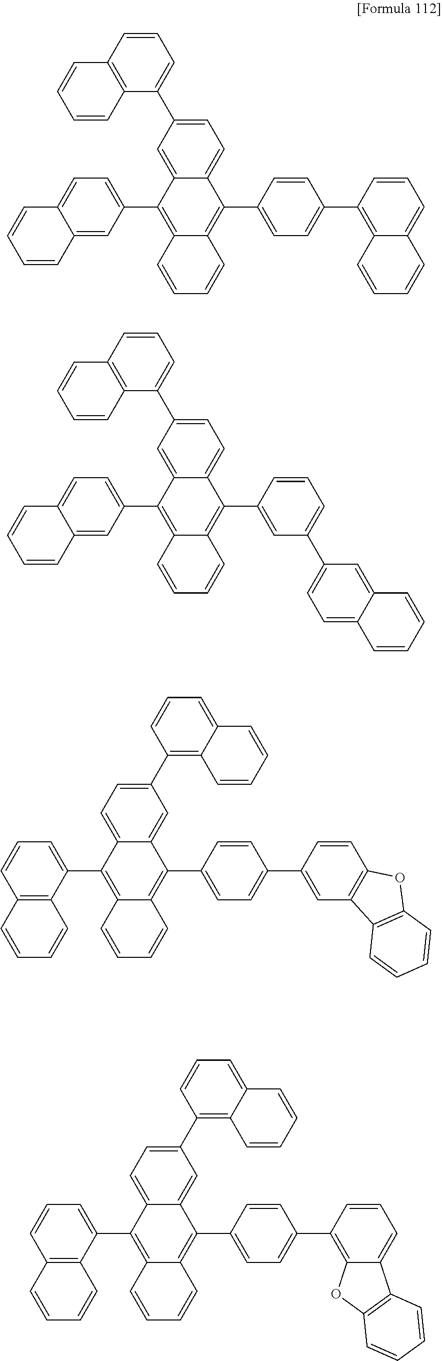

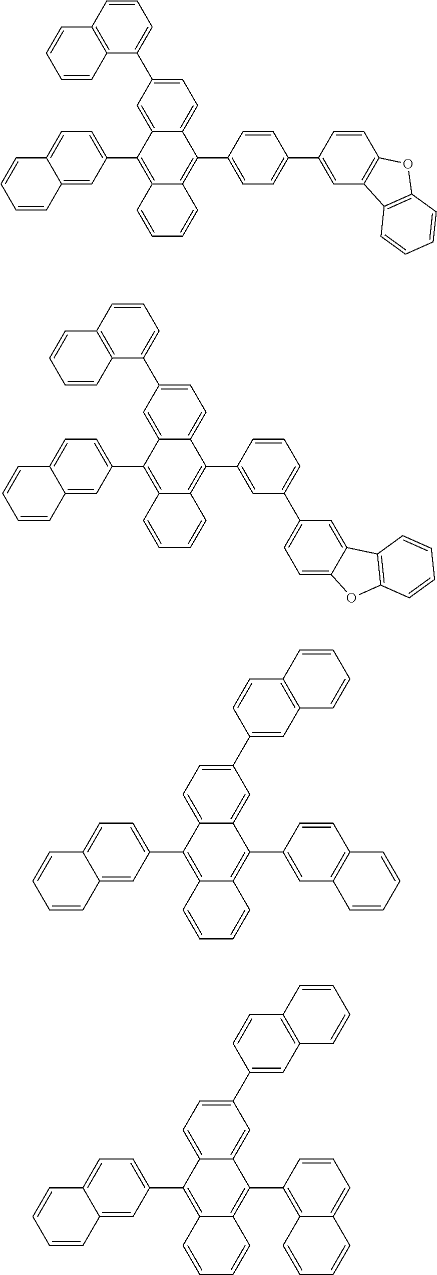

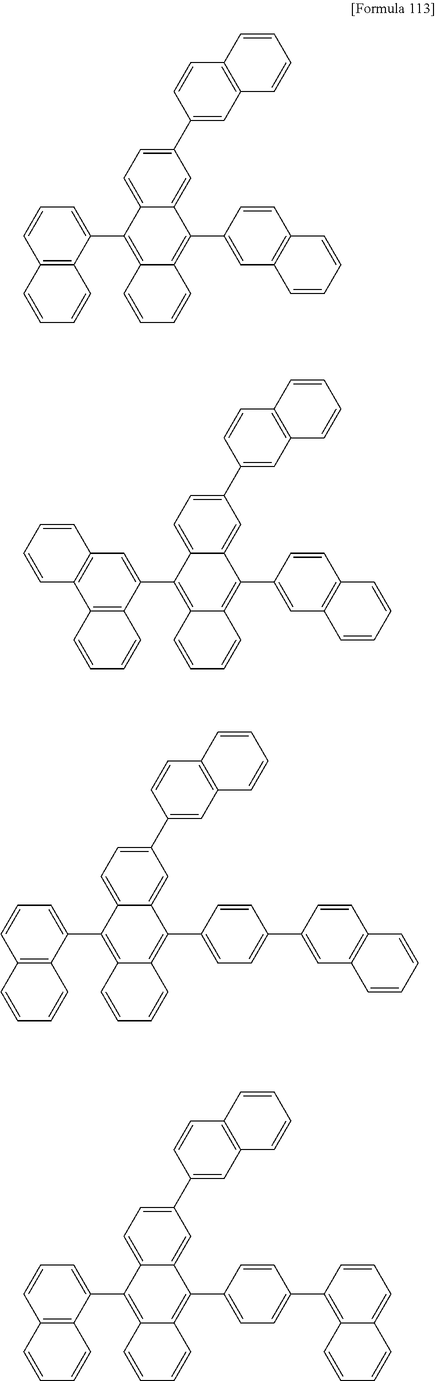

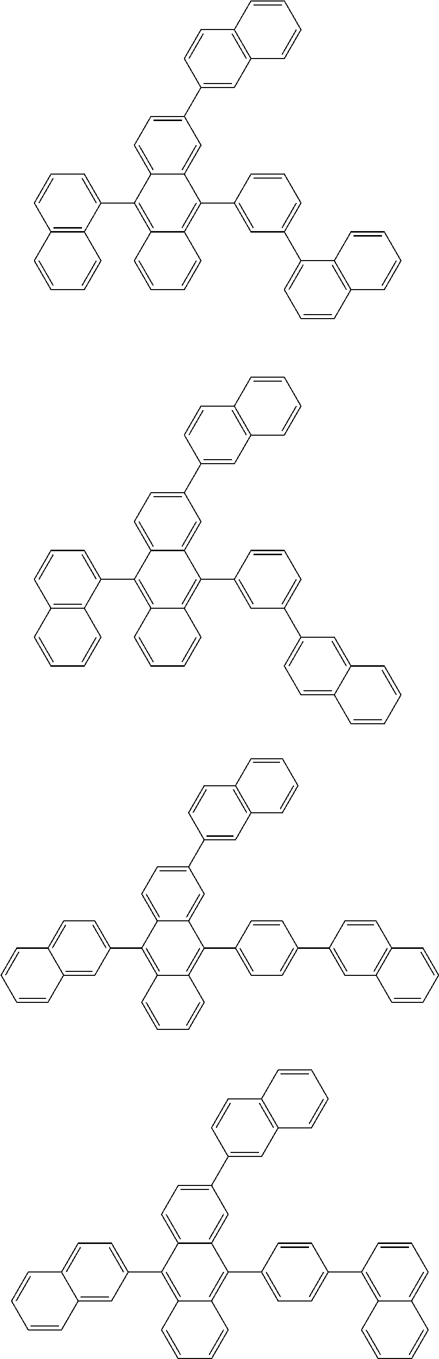

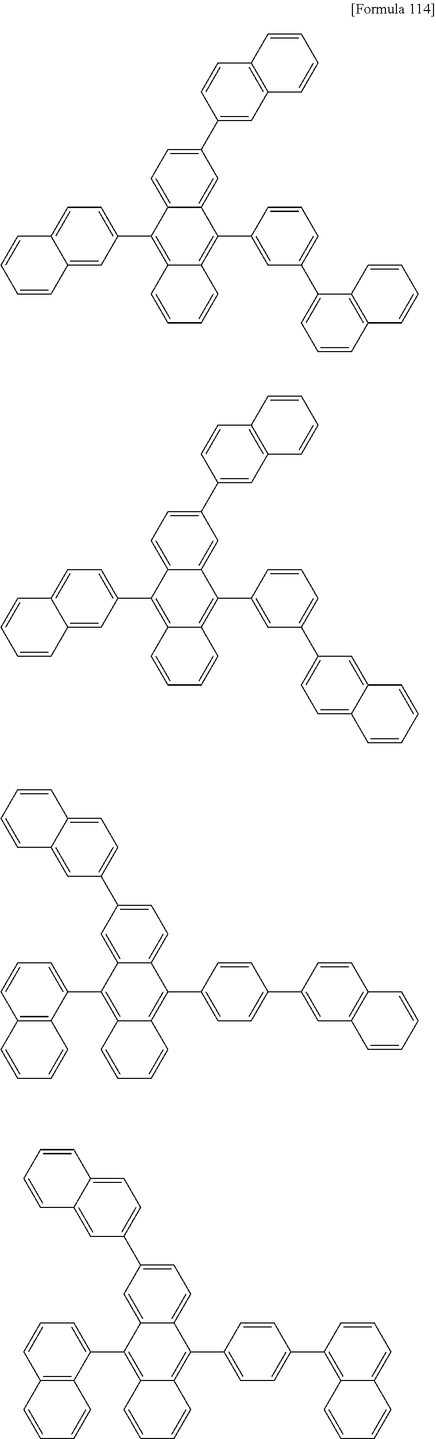

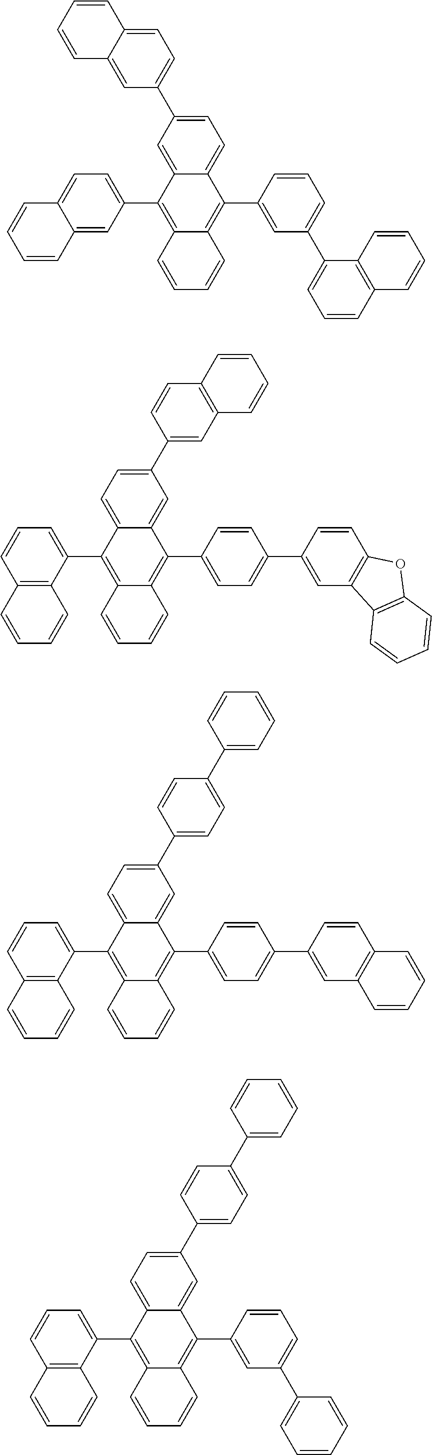

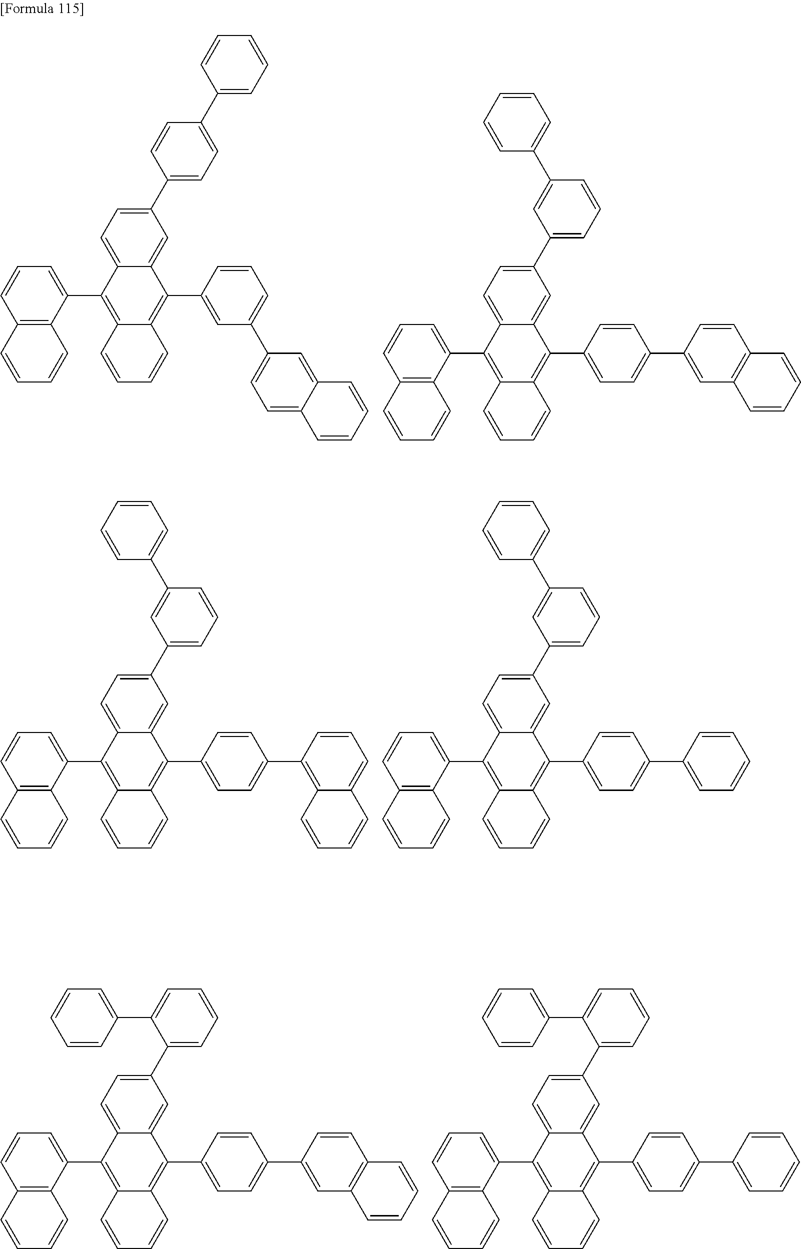

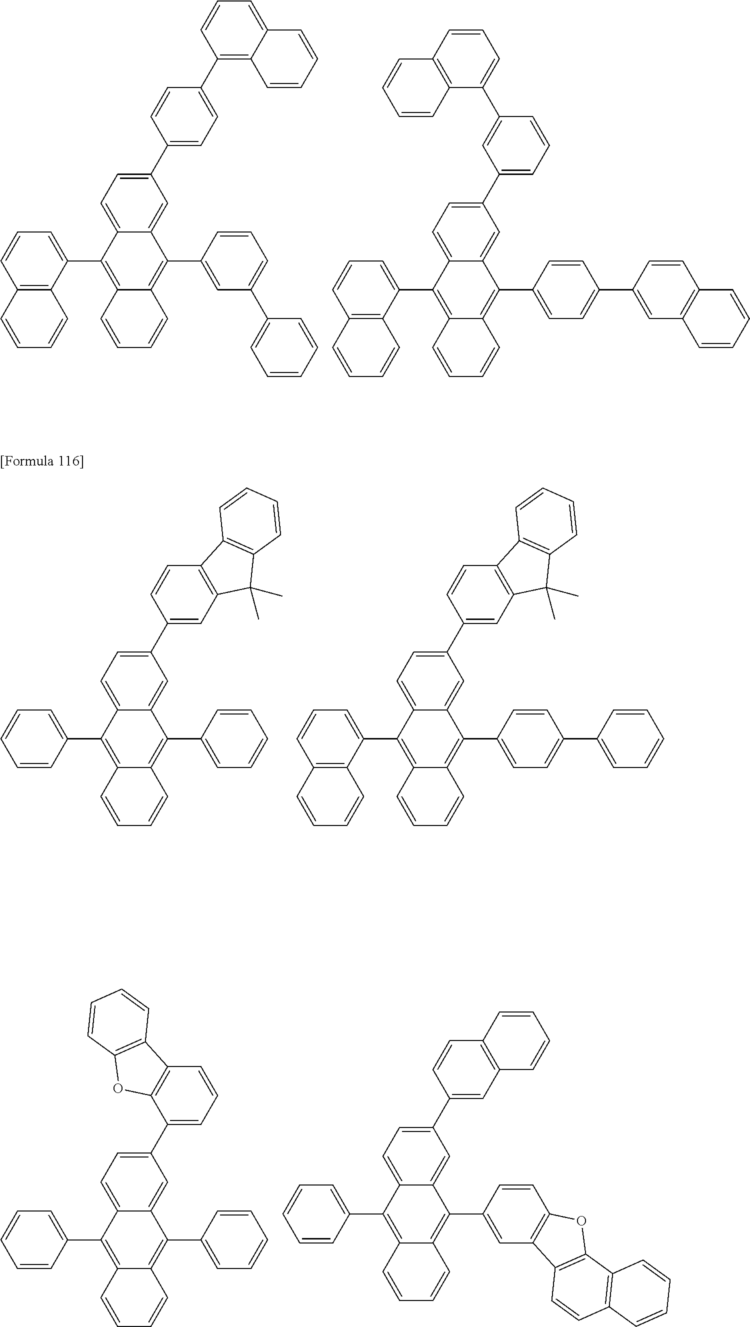

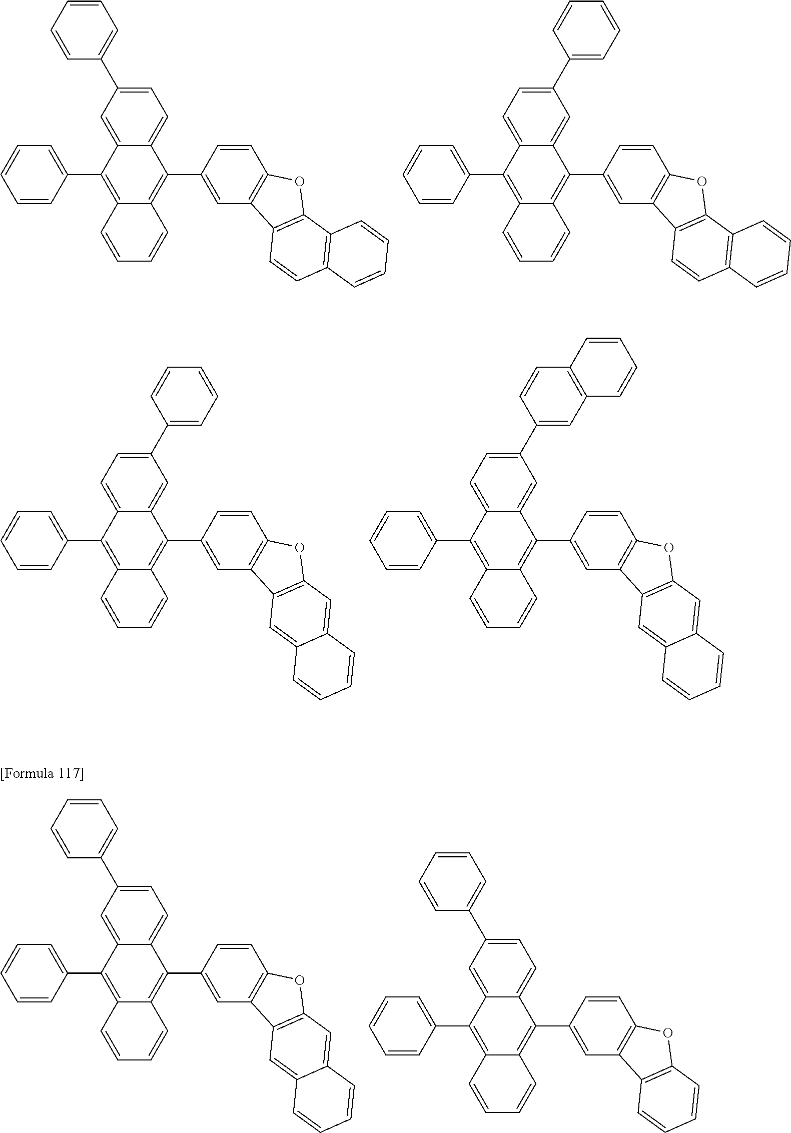

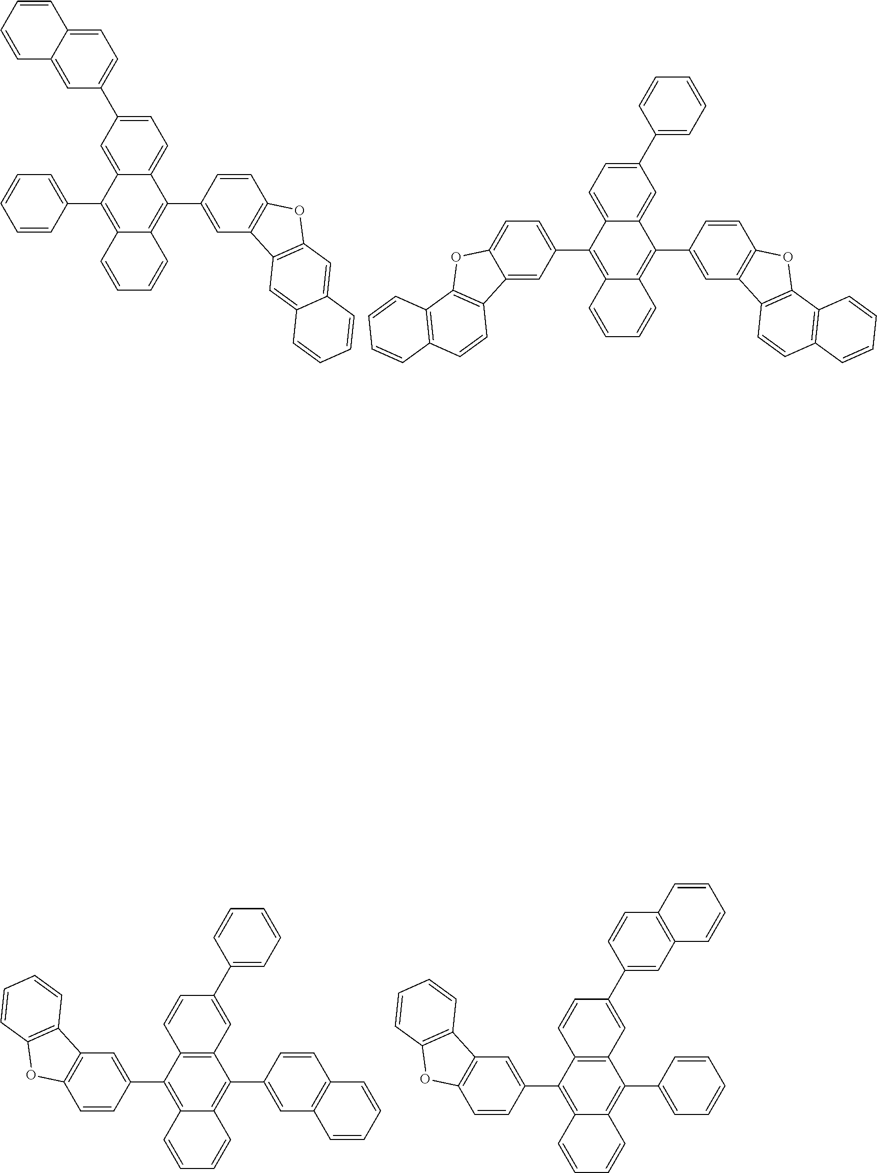

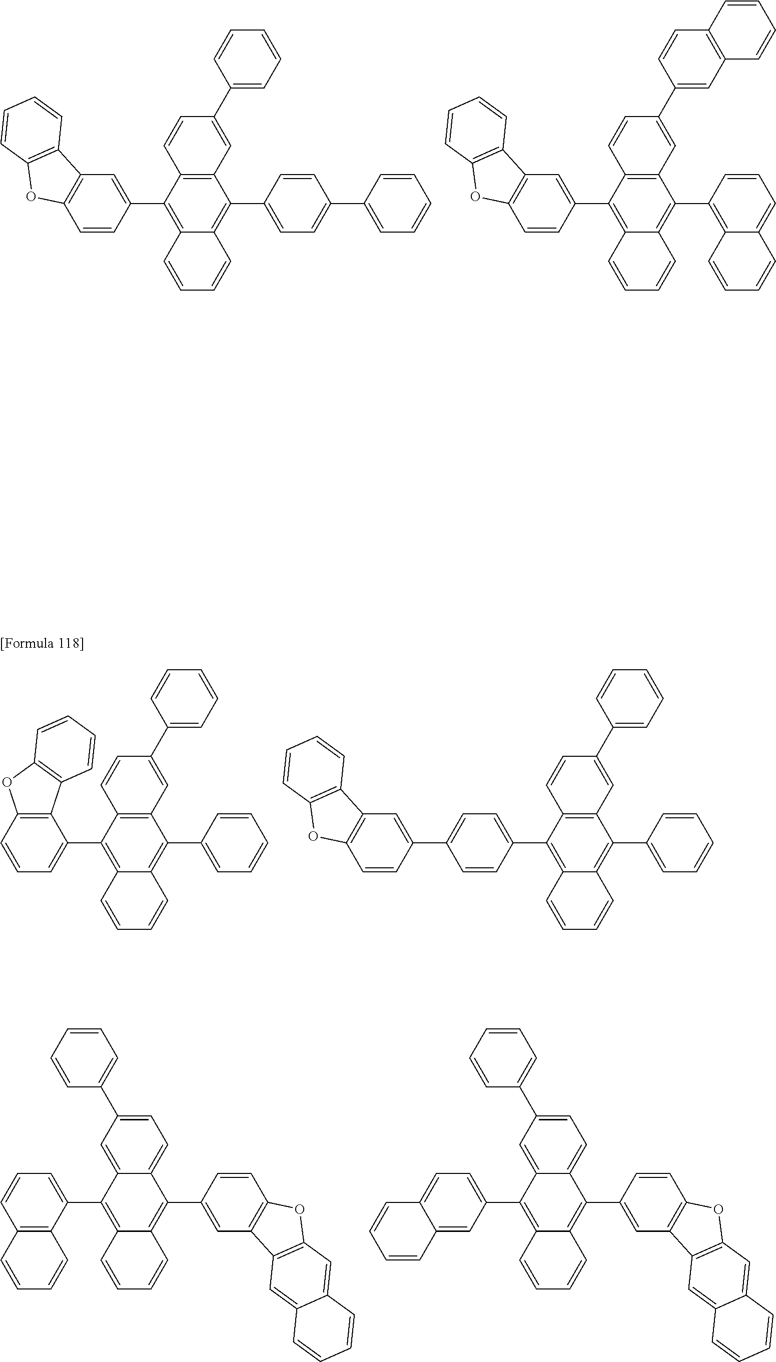

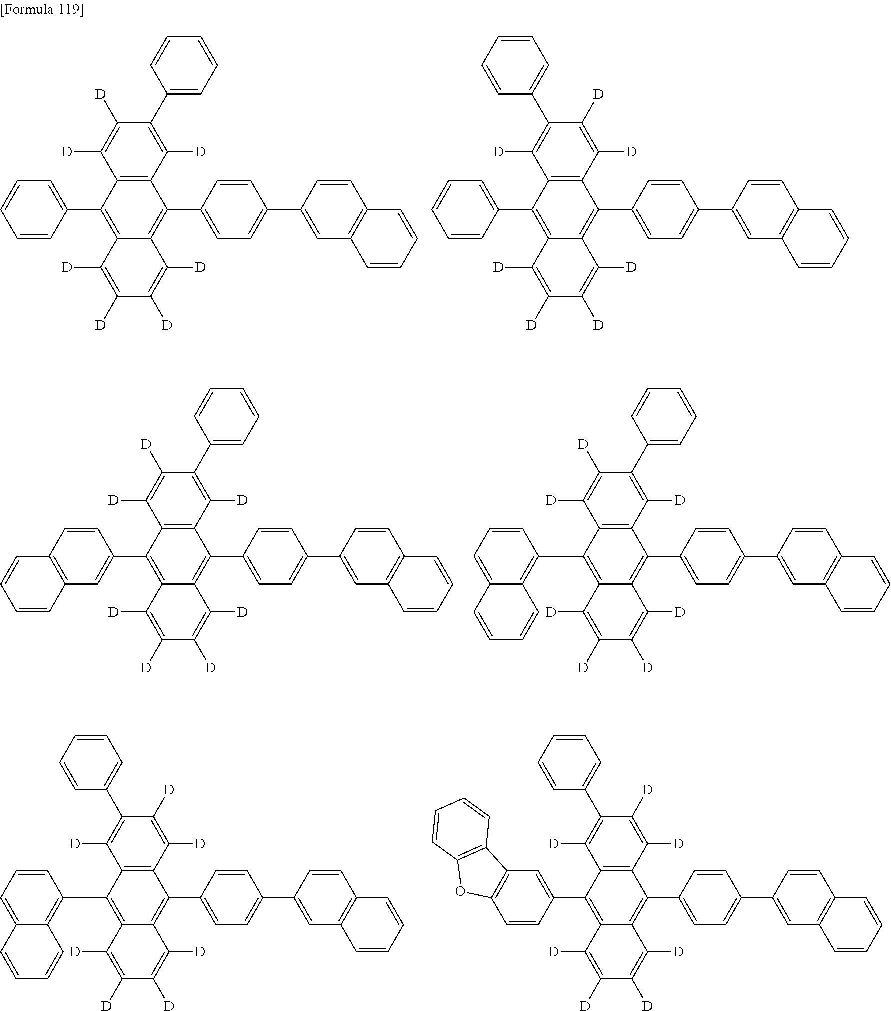

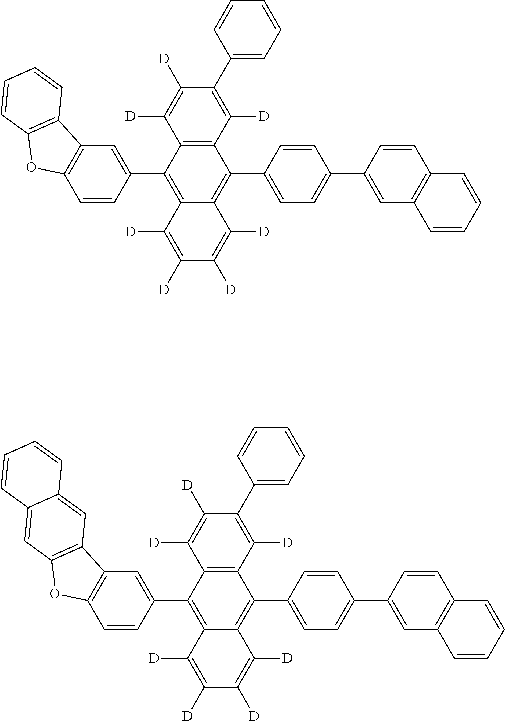 [SMILES: [2H]c1c([2H])c([2H])c2c(-c3ccc(-c4ccc5ccccc5c4)cc3)c3c([2H])c(-c4ccccc4)c([2H])c([2H])c3c(-c3ccc4oc5ccccc5c4c3)c2c1[2H] ; [2H]c1c([2H])c([2H])c2c(-c3ccc(-c4ccc5ccccc5c4)cc3)c3c([2H])c(-c4ccccc4)c([2H])c([2H])c3c(-c3ccc4oc5cc6ccccc6cc5c4c3)c2c1[2H]]

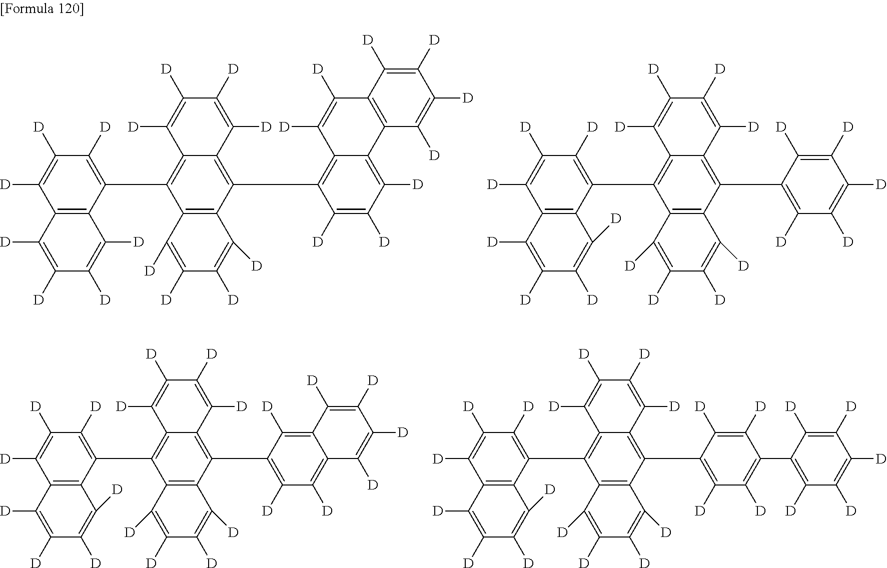

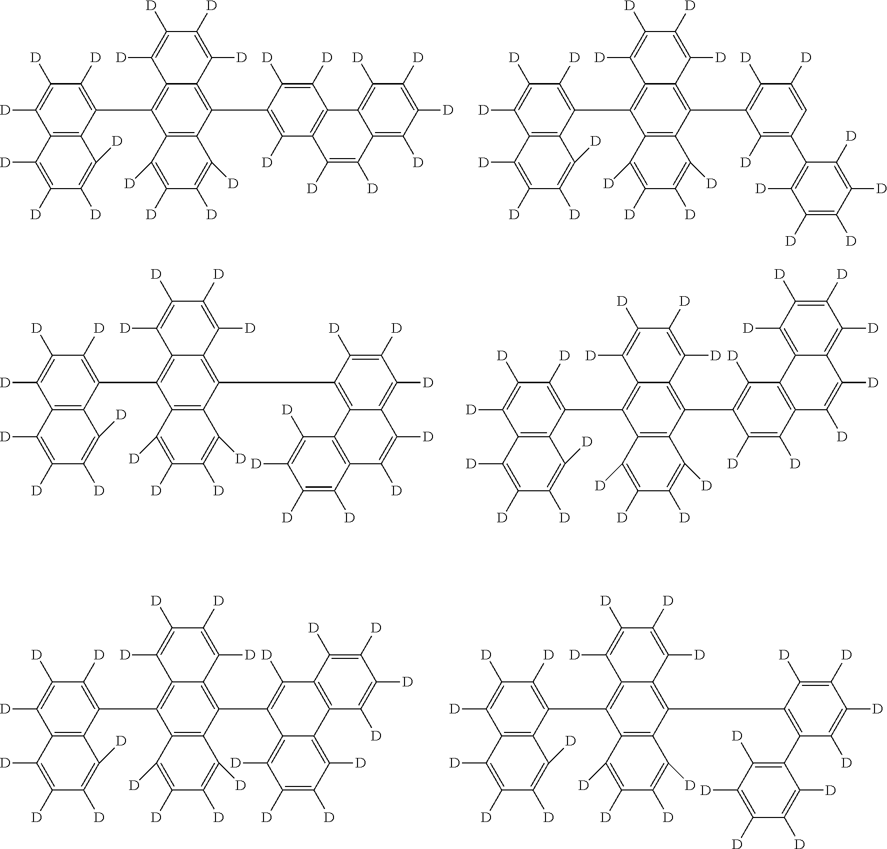

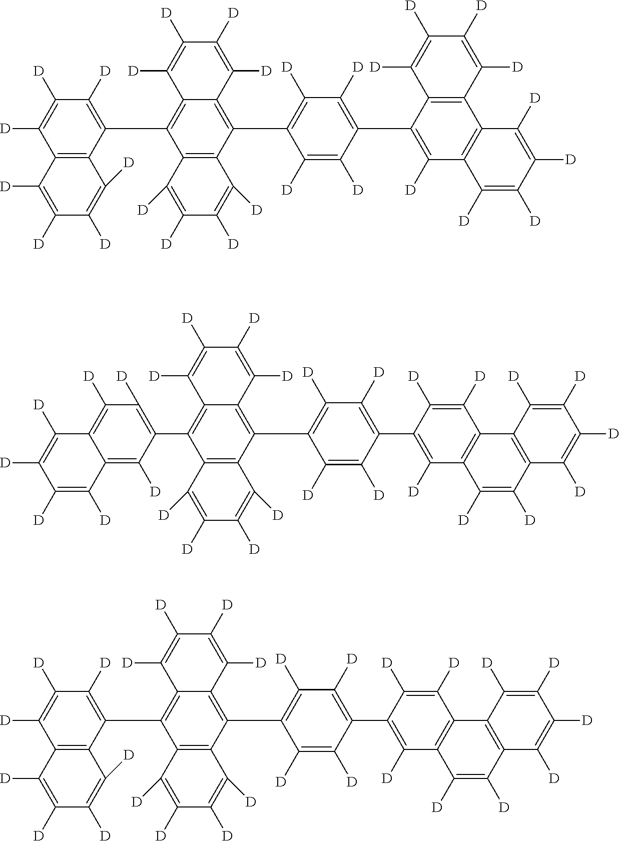

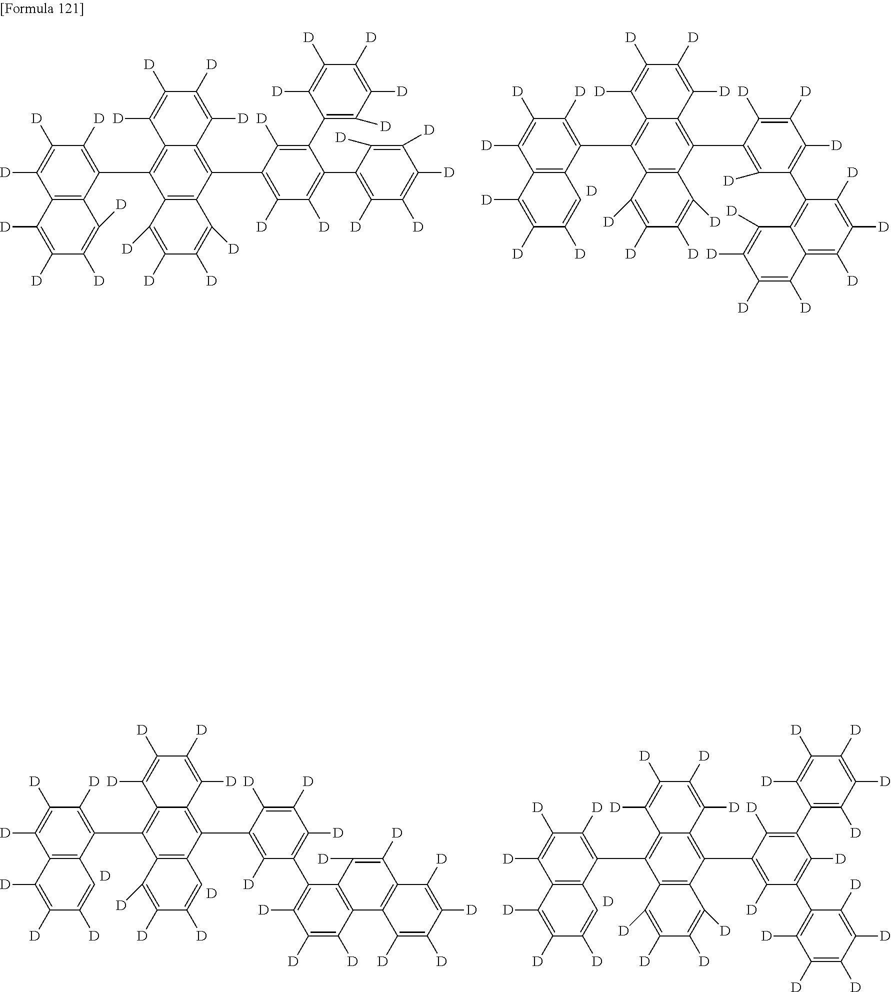

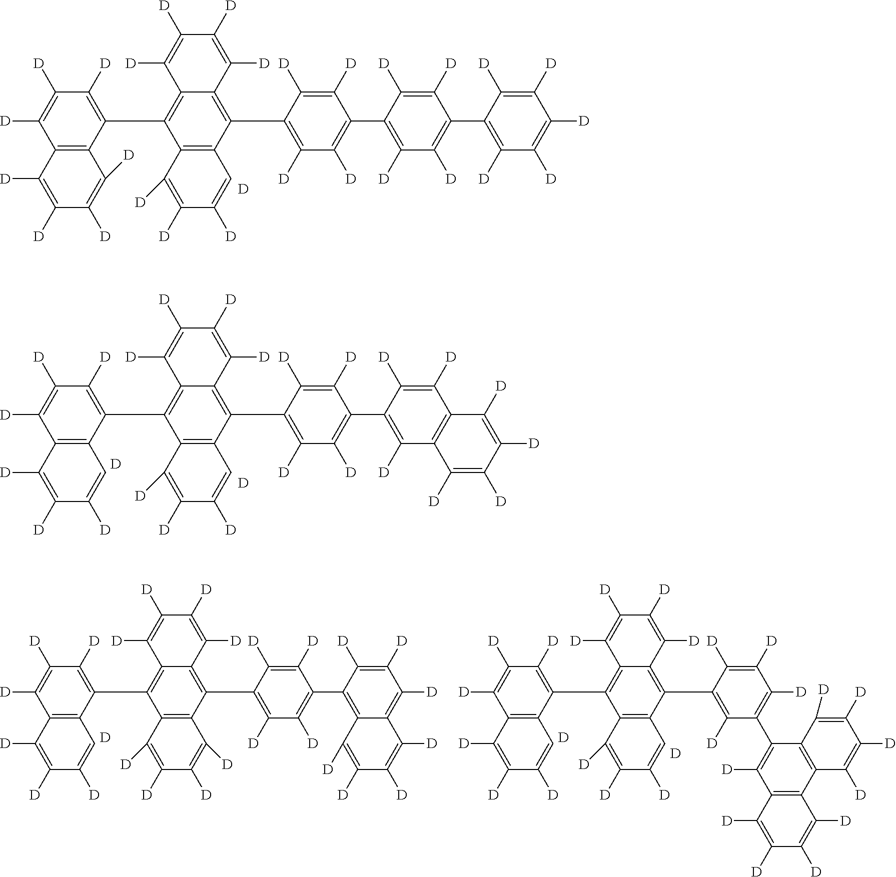

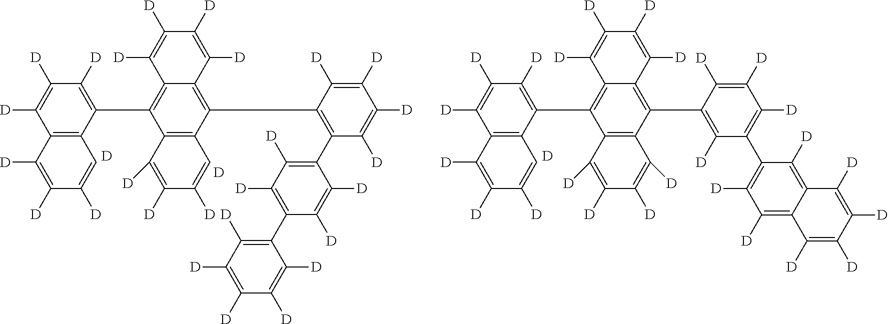

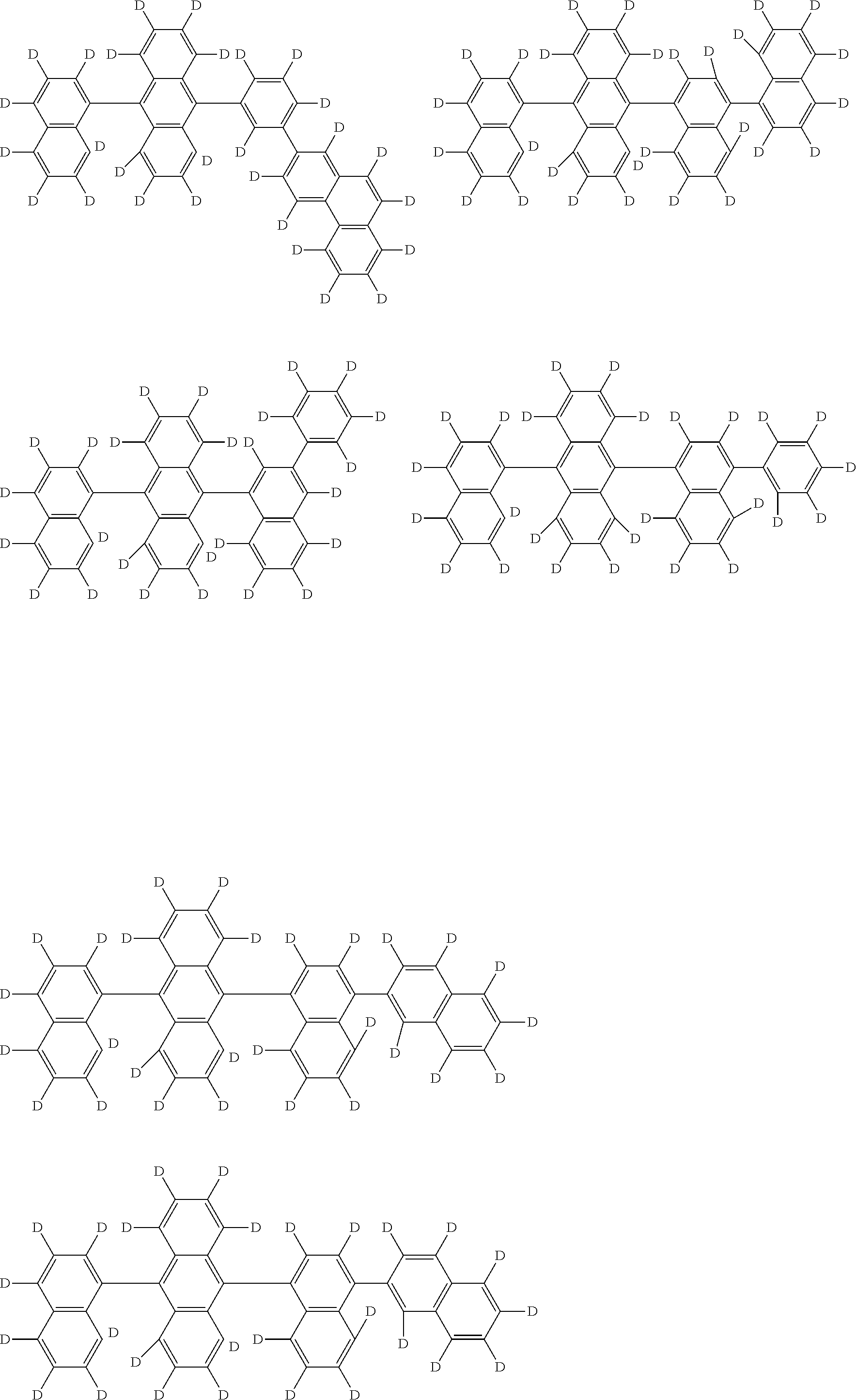

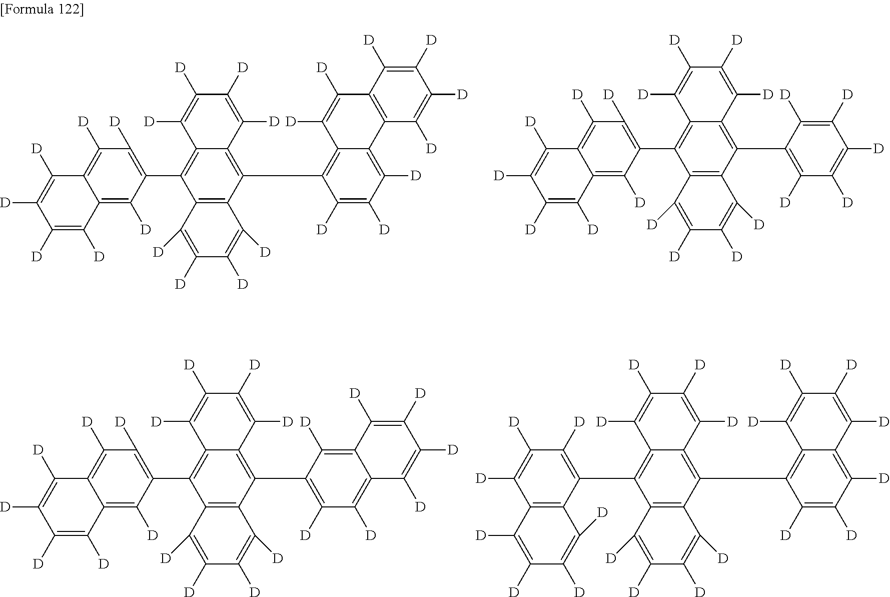

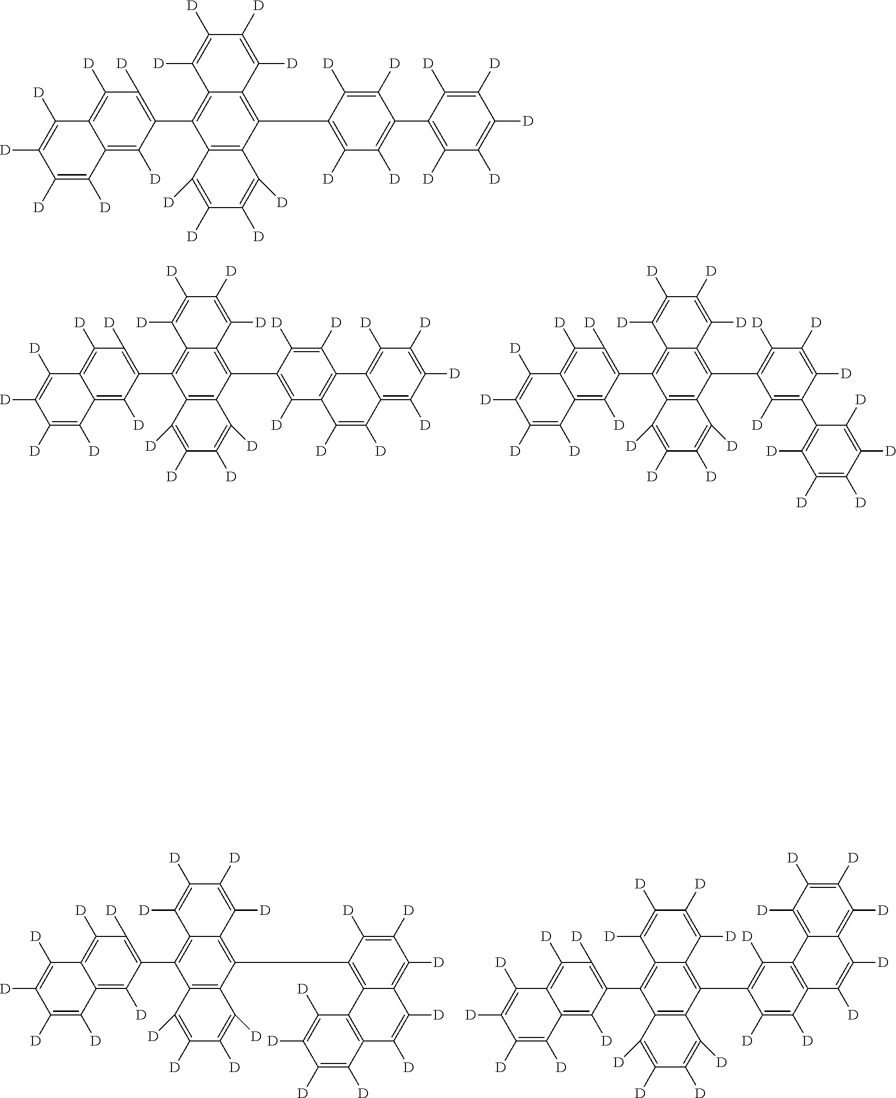

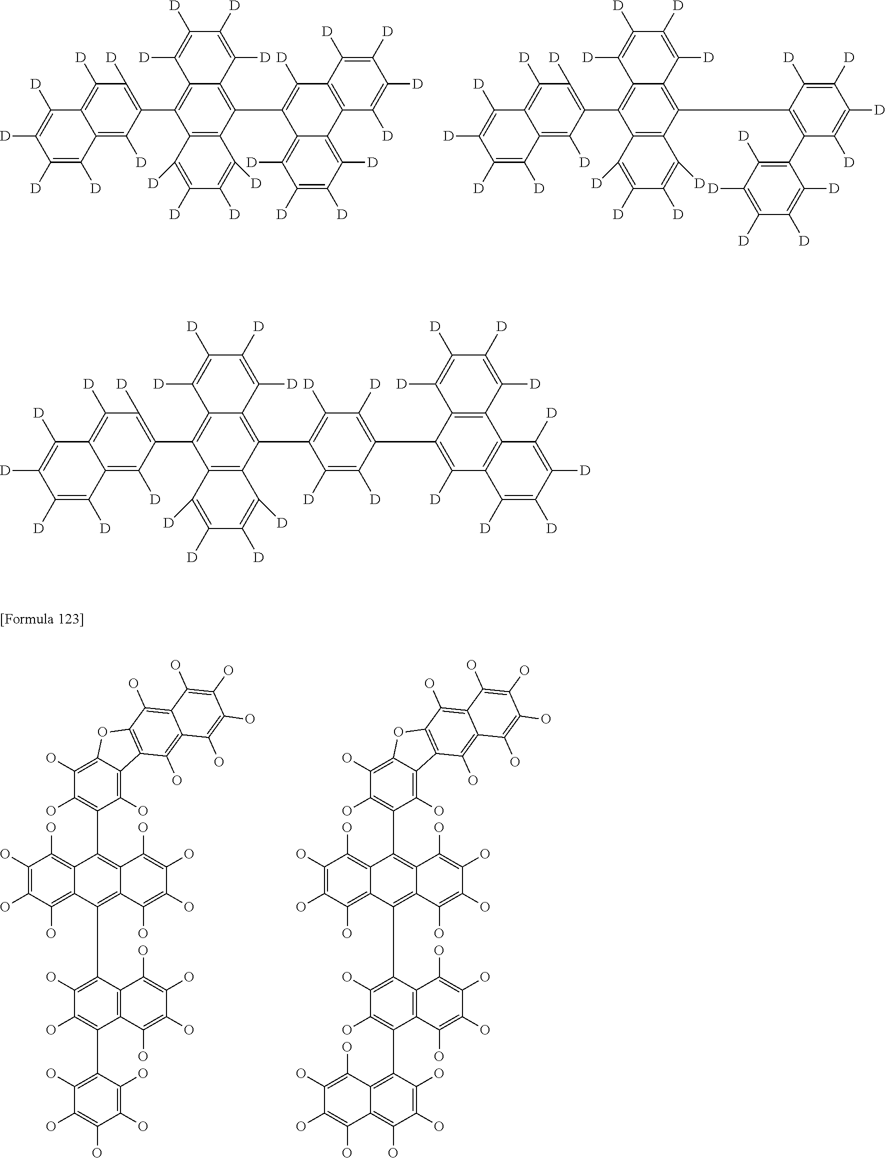

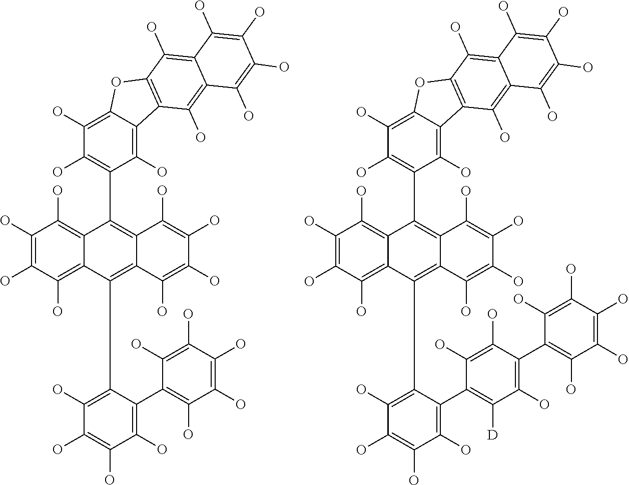 [SMILES: Oc1c(O)c(O)c(-c2c(O)c(O)c(O)c(O)c2-c2c3c(O)c(O)c(O)c(O)c3c(-c3c(O)c(O)c4oc5c(O)c6c(O)c(O)c(O)c(O)c6c(O)c5c4c3O)c3c(O)c(O)c(O)c(O)c23)c(O)c1O ; [2H]c1c(O)c(-c2c(O)c(O)c(O)c(O)c2O)c(O)c(O)c1-c1c(O)c(O)c(O)c(O)c1-c1c2c(O)c(O)c(O)c(O)c2c(-c2c(O)c(O)c3oc4c(O)c5c(O)c(O)c(O)c(O)c5c(O)c4c3c2O)c2c(O)c(O)c(O)c(O)c12]

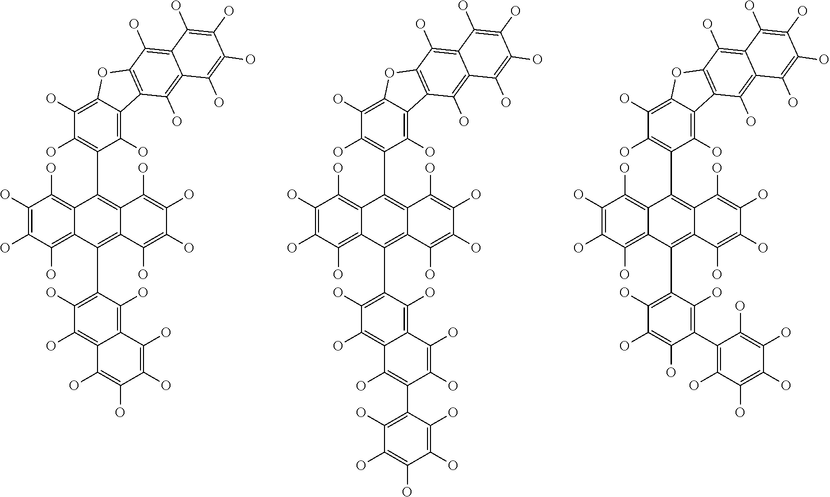 [SMILES: Oc1c(-c2c3c(O)c(O)c(O)c(O)c3c(-c3c(O)c(O)c4c(O)c(O)c(O)c(O)c4c3O)c3c(O)c(O)c(O)c(O)c23)c(O)c2c(oc3c(O)c4c(O)c(O)c(O)c(O)c4c(O)c32)c1O ; Oc1c(O)c(O)c(-c2c(O)c(O)c3c(O)c(-c4c5c(O)c(O)c(O)c(O)c5c(-c5c(O)c(O)c6oc7c(O)c8c(O)c(O)c(O)c(O)c8c(O)c7c6c5O)c5c(O)c(O)c(O)c(O)c45)c(O)c(O)c3c2O)c(O)c1O ; Oc1c(O)c(O)c(-c2c(O)c(O)c(O)c(-c3c4c(O)c(O)c(O)c(O)c4c(-c4c(O)c(O)c5oc6c(O)c7c(O)c(O)c(O)c(O)c7c(O)c6c5c4O)c4c(O)c(O)c(O)c(O)c34)c2O)c(O)c1O]

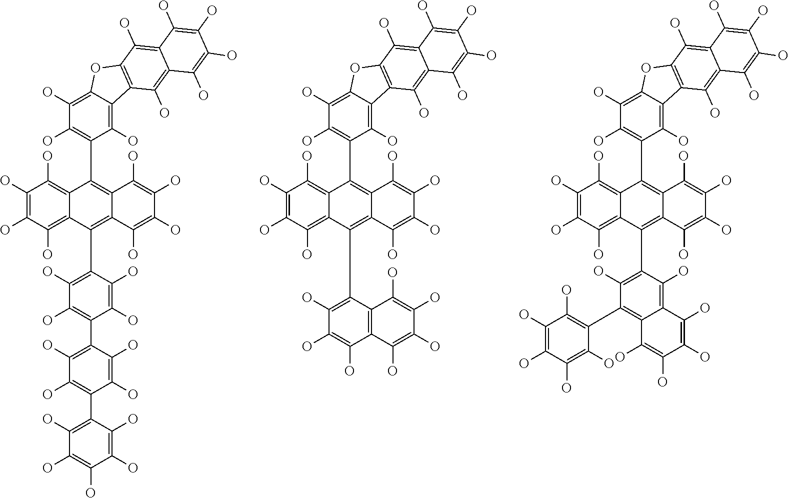 [SMILES: Oc1c(O)c(O)c(-c2c(O)c(O)c(-c3c(O)c(O)c(-c4c5c(O)c(O)c(O)c(O)c5c(-c5c(O)c(O)c6oc7c(O)c8c(O)c(O)c(O)c(O)c8c(O)c7c6c5O)c5c(O)c(O)c(O)c(O)c45)c(O)c3O)c(O)c2O)c(O)c1O ; Oc1c(-c2c3c(O)c(O)c(O)c(O)c3c(-c3c(O)c(O)c(O)c4c(O)c(O)c(O)c(O)c34)c3c(O)c(O)c(O)c(O)c23)c(O)c2c(oc3c(O)c4c(O)c(O)c(O)c(O)c4c(O)c32)c1O ; Oc1c(O)c(O)c(-c2c(O)c(-c3c4c(O)c(O)c(O)c(O)c4c(-c4c(O)c(O)c5oc6c(O)c7c(O)c(O)c(O)c(O)c7c(O)c6c5c4O)c4c(O)c(O)c(O)c(O)c34)c(O)c3c(O)c(O)c(O)c(O)c23)c(O)c1O]

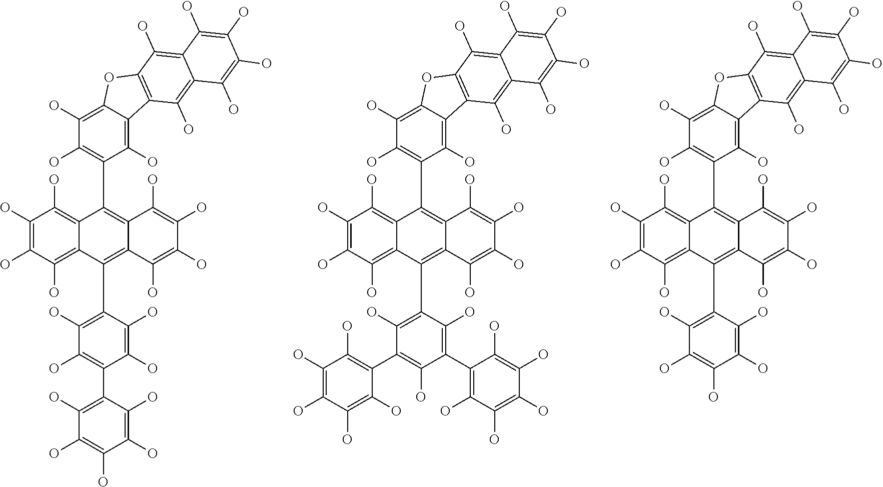 [SMILES: Oc1c(O)c(O)c(-c2c(O)c(O)c(-c3c4c(O)c(O)c(O)c(O)c4c(-c4c(O)c(O)c5oc6c(O)c7c(O)c(O)c(O)c(O)c7c(O)c6c5c4O)c4c(O)c(O)c(O)c(O)c34)c(O)c2O)c(O)c1O ; Oc1c(O)c(O)c(-c2c(O)c(-c3c(O)c(O)c(O)c(O)c3O)c(O)c(-c3c4c(O)c(O)c(O)c(O)c4c(-c4c(O)c(O)c5oc6c(O)c7c(O)c(O)c(O)c(O)c7c(O)c6c5c4O)c4c(O)c(O)c(O)c(O)c34)c2O)c(O)c1O ; Oc1c(O)c(O)c(-c2c3c(O)c(O)c(O)c(O)c3c(-c3c(O)c(O)c4oc5c(O)c6c(O)c(O)c(O)c(O)c6c(O)c5c4c3O)c3c(O)c(O)c(O)c(O)c23)c(O)c1O]

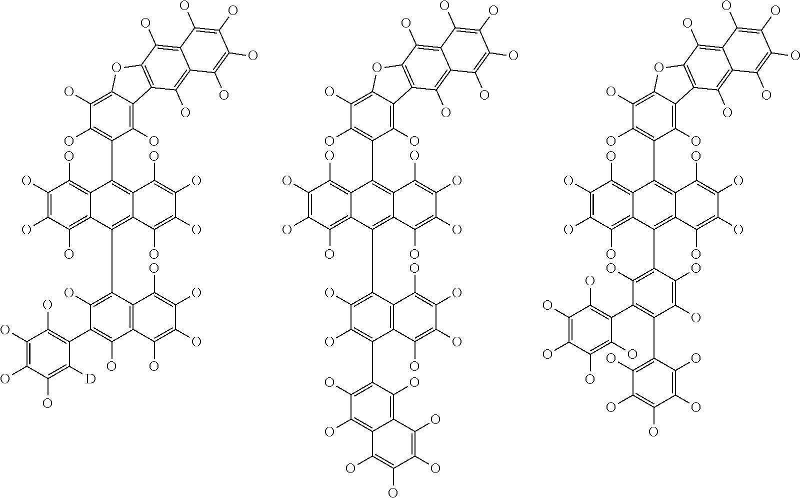 [SMILES: [2H]c1c(O)c(O)c(O)c(O)c1-c1c(O)c(-c2c3c(O)c(O)c(O)c(O)c3c(-c3c(O)c(O)c4oc5c(O)c6c(O)c(O)c(O)c(O)c6c(O)c5c4c3O)c3c(O)c(O)c(O)c(O)c23)c2c(O)c(O)c(O)c(O)c2c1O ; Oc1c(-c2c3c(O)c(O)c(O)c(O)c3c(-c3c(O)c(O)c(-c4c(O)c(O)c5c(O)c(O)c(O)c(O)c5c4O)c4c(O)c(O)c(O)c(O)c34)c3c(O)c(O)c(O)c(O)c23)c(O)c2c(oc3c(O)c4c(O)c(O)c(O)c(O)c4c(O)c32)c1O ; Oc1c(O)c(O)c(-c2c(O)c(O)c(-c3c4c(O)c(O)c(O)c(O)c4c(-c4c(O)c(O)c5oc6c(O)c7c(O)c(O)c(O)c(O)c7c(O)c6c5c4O)c4c(O)c(O)c(O)c(O)c34)c(O)c2-c2c(O)c(O)c(O)c(O)c2O)c(O)c1O]

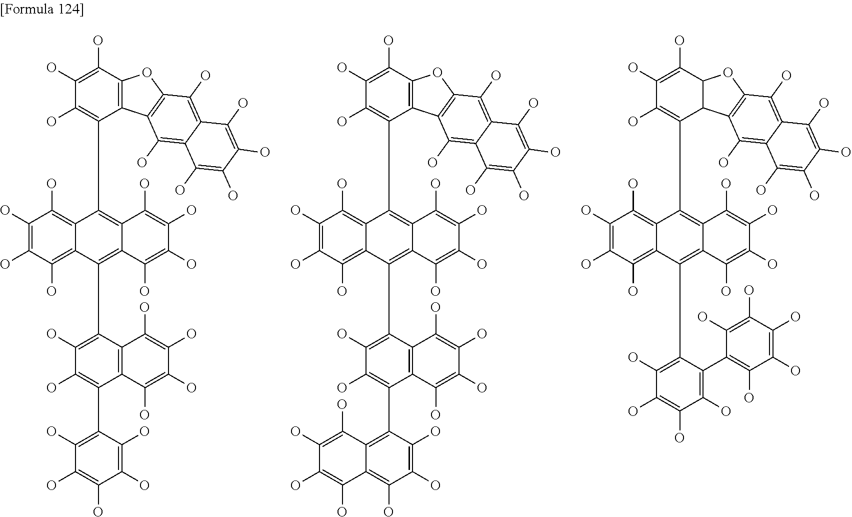 [SMILES: Oc1c(O)c(O)c(-c2c(O)c(O)c(-c3c4c(O)c(O)c(O)c(O)c4c(-c4c(O)c(O)c(O)c5oc6c(O)c7c(O)c(O)c(O)c(O)c7c(O)c6c45)c4c(O)c(O)c(O)c(O)c34)c3c(O)c(O)c(O)c(O)c23)c(O)c1O ; Oc1c(O)c(-c2c3c(O)c(O)c(O)c(O)c3c(-c3c(O)c(O)c(-c4c(O)c(O)c(O)c5c(O)c(O)c(O)c(O)c45)c4c(O)c(O)c(O)c(O)c34)c3c(O)c(O)c(O)c(O)c23)c2c(oc3c(O)c4c(O)c(O)c(O)c(O)c4c(O)c32)c1O ; OC1=C(O)C2Oc3c(c(O)c4c(O)c(O)c(O)c(O)c4c3O)C2C(c2c3c(O)c(O)c(O)c(O)c3c(-c3c(O)c(O)c(O)c(O)c3-c3c(O)c(O)c(O)c(O)c3O)c3c(O)c(O)c(O)c(O)c23)=C1O]

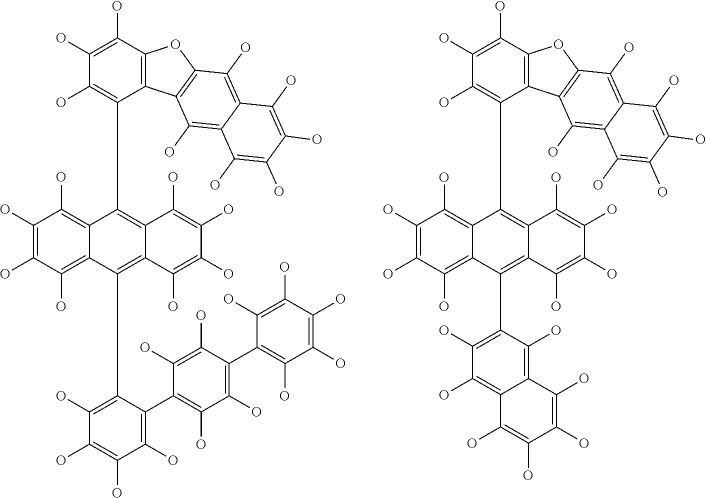 [SMILES: Oc1c(O)c(O)c(-c2c(O)c(O)c(-c3c(O)c(O)c(O)c(O)c3-c3c4c(O)c(O)c(O)c(O)c4c(-c4c(O)c(O)c(O)c5oc6c(O)c7c(O)c(O)c(O)c(O)c7c(O)c6c45)c4c(O)c(O)c(O)c(O)c34)c(O)c2O)c(O)c1O ; Oc1c(O)c(-c2c3c(O)c(O)c(O)c(O)c3c(-c3c(O)c(O)c4c(O)c(O)c(O)c(O)c4c3O)c3c(O)c(O)c(O)c(O)c23)c2c(oc3c(O)c4c(O)c(O)c(O)c(O)c4c(O)c32)c1O]

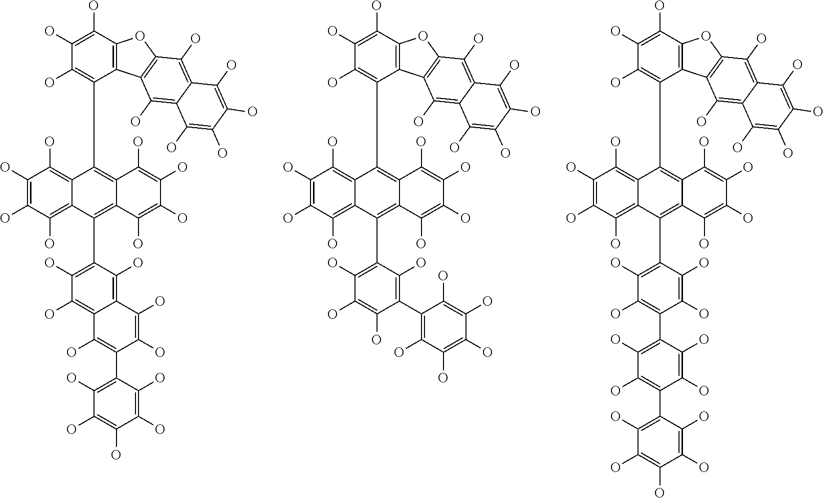 [SMILES: Oc1c(O)c(O)c(-c2c(O)c(O)c3c(O)c(-c4c5c(O)c(O)c(O)c(O)c5c(-c5c(O)c(O)c(O)c6oc7c(O)c8c(O)c(O)c(O)c(O)c8c(O)c7c56)c5c(O)c(O)c(O)c(O)c45)c(O)c(O)c3c2O)c(O)c1O ; Oc1c(O)c(O)c(-c2c(O)c(O)c(O)c(-c3c4c(O)c(O)c(O)c(O)c4c(-c4c(O)c(O)c(O)c5oc6c(O)c7c(O)c(O)c(O)c(O)c7c(O)c6c45)c4c(O)c(O)c(O)c(O)c34)c2O)c(O)c1O ; Oc1c(O)c(O)c(-c2c(O)c(O)c(-c3c(O)c(O)c(-c4c5c(O)c(O)c(O)c(O)c5c(-c5c(O)c(O)c(O)c6oc7c(O)c8c(O)c(O)c(O)c(O)c8c(O)c7c56)c5c(O)c(O)c(O)c(O)c45)c(O)c3O)c(O)c2O)c(O)c1O]

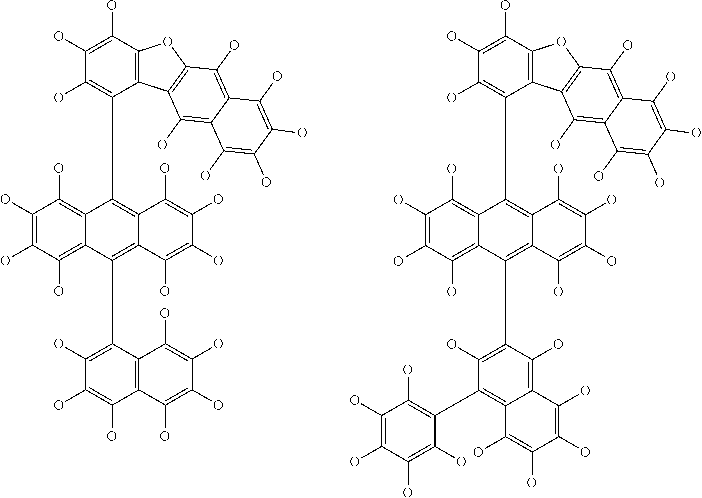 [SMILES: Oc1c(O)c(-c2c3c(O)c(O)c(O)c(O)c3c(-c3c(O)c(O)c(O)c4c(O)c(O)c(O)c(O)c34)c3c(O)c(O)c(O)c(O)c23)c2c(oc3c(O)c4c(O)c(O)c(O)c(O)c4c(O)c32)c1O ; Oc1c(O)c(O)c(-c2c(O)c(-c3c4c(O)c(O)c(O)c(O)c4c(-c4c(O)c(O)c(O)c5oc6c(O)c7c(O)c(O)c(O)c(O)c7c(O)c6c45)c4c(O)c(O)c(O)c(O)c34)c(O)c3c(O)c(O)c(O)c(O)c23)c(O)c1O]

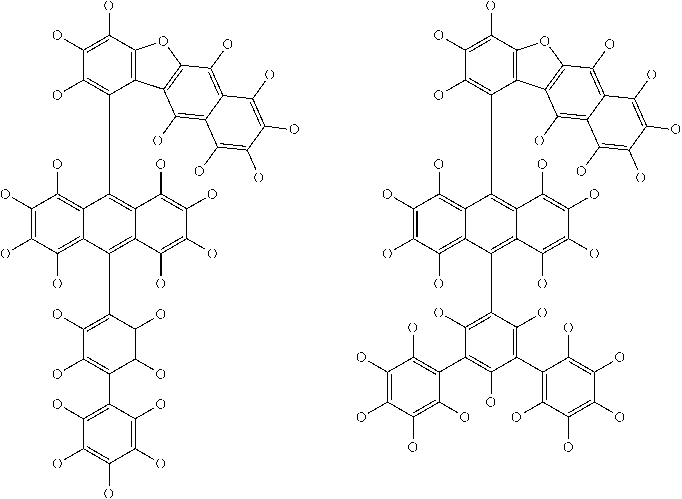 [SMILES: OC1=C(c2c(O)c(O)c(O)c(O)c2O)C(O)C(O)C(c2c3c(O)c(O)c(O)c(O)c3c(-c3c(O)c(O)c(O)c4oc5c(O)c6c(O)c(O)c(O)c(O)c6c(O)c5c34)c3c(O)c(O)c(O)c(O)c23)=C1O ; Oc1c(O)c(O)c(-c2c(O)c(-c3c(O)c(O)c(O)c(O)c3O)c(O)c(-c3c4c(O)c(O)c(O)c(O)c4c(-c4c(O)c(O)c(O)c5oc6c(O)c7c(O)c(O)c(O)c(O)c7c(O)c6c45)c4c(O)c(O)c(O)c(O)c34)c2O)c(O)c1O]

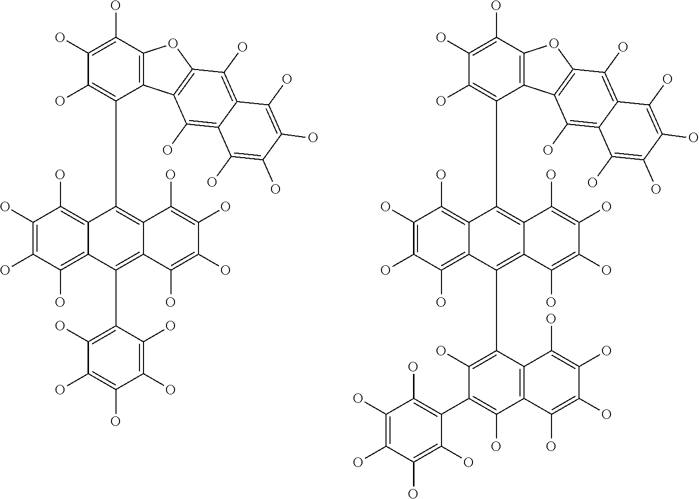 [SMILES: Oc1c(O)c(O)c(-c2c3c(O)c(O)c(O)c(O)c3c(-c3c(O)c(O)c(O)c4oc5c(O)c6c(O)c(O)c(O)c(O)c6c(O)c5c34)c3c(O)c(O)c(O)c(O)c23)c(O)c1O ; Oc1c(O)c(O)c(-c2c(O)c(-c3c4c(O)c(O)c(O)c(O)c4c(-c4c(O)c(O)c(O)c5oc6c(O)c7c(O)c(O)c(O)c(O)c7c(O)c6c45)c4c(O)c(O)c(O)c(O)c34)c3c(O)c(O)c(O)c(O)c3c2O)c(O)c1O]

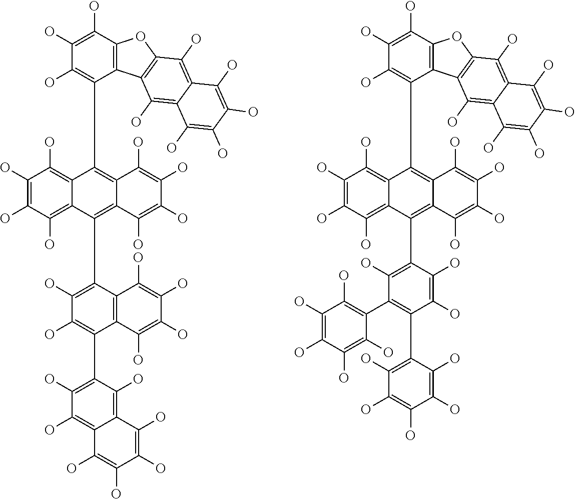 [SMILES: Oc1c(O)c(-c2c3c(O)c(O)c(O)c(O)c3c(-c3c(O)c(O)c(-c4c(O)c(O)c5c(O)c(O)c(O)c(O)c5c4O)c4c(O)c(O)c(O)c(O)c34)c3c(O)c(O)c(O)c(O)c23)c2c(oc3c(O)c4c(O)c(O)c(O)c(O)c4c(O)c32)c1O ; Oc1c(O)c(O)c(-c2c(O)c(O)c(-c3c4c(O)c(O)c(O)c(O)c4c(-c4c(O)c(O)c(O)c5oc6c(O)c7c(O)c(O)c(O)c(O)c7c(O)c6c45)c4c(O)c(O)c(O)c(O)c34)c(O)c2-c2c(O)c(O)c(O)c(O)c2O)c(O)c1O]

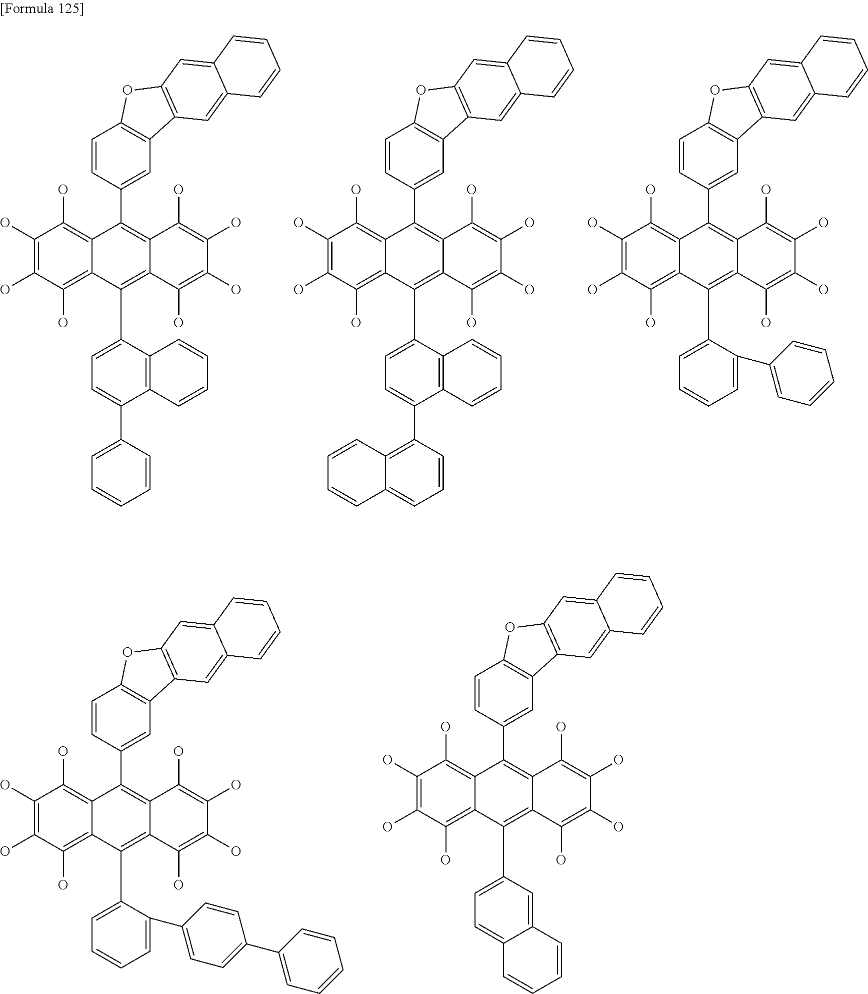 [SMILES: Oc1c(O)c(O)c2c(-c3ccc(-c4ccccc4)c4ccccc34)c3c(O)c(O)c(O)c(O)c3c(-c3ccc4oc5cc6ccccc6cc5c4c3)c2c1O ; Oc1c(O)c(O)c2c(-c3ccc(-c4cccc5ccccc45)c4ccccc34)c3c(O)c(O)c(O)c(O)c3c(-c3ccc4oc5cc6ccccc6cc5c4c3)c2c1O ; Oc1c(O)c(O)c2c(-c3ccccc3-c3ccccc3)c3c(O)c(O)c(O)c(O)c3c(-c3ccc4oc5cc6ccccc6cc5c4c3)c2c1O ; Oc1c(O)c(O)c2c(-c3ccccc3-c3ccc(-c4ccccc4)cc3)c3c(O)c(O)c(O)c(O)c3c(-c3ccc4oc5cc6ccccc6cc5c4c3)c2c1O ; Oc1c(O)c(O)c2c(-c3ccc4oc5cc6ccccc6cc5c4c3)c3c(O)c(O)c(O)c(O)c3c(-c3ccc4ccccc4c3)c2c1O]

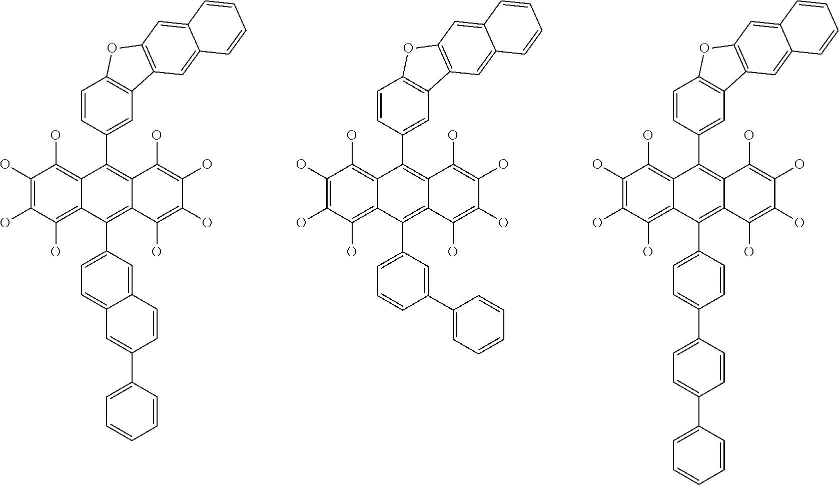 [SMILES: Oc1c(O)c(O)c2c(-c3ccc4oc5cc6ccccc6cc5c4c3)c3c(O)c(O)c(O)c(O)c3c(-c3ccc4cc(-c5ccccc5)ccc4c3)c2c1O ; Oc1c(O)c(O)c2c(-c3ccc4oc5cc6ccccc6cc5c4c3)c3c(O)c(O)c(O)c(O)c3c(-c3cccc(-c4ccccc4)c3)c2c1O ; Oc1c(O)c(O)c2c(-c3ccc4oc5cc6ccccc6cc5c4c3)c3c(O)c(O)c(O)c(O)c3c(-c3ccc(-c4ccc(-c5ccccc5)cc4)cc3)c2c1O]

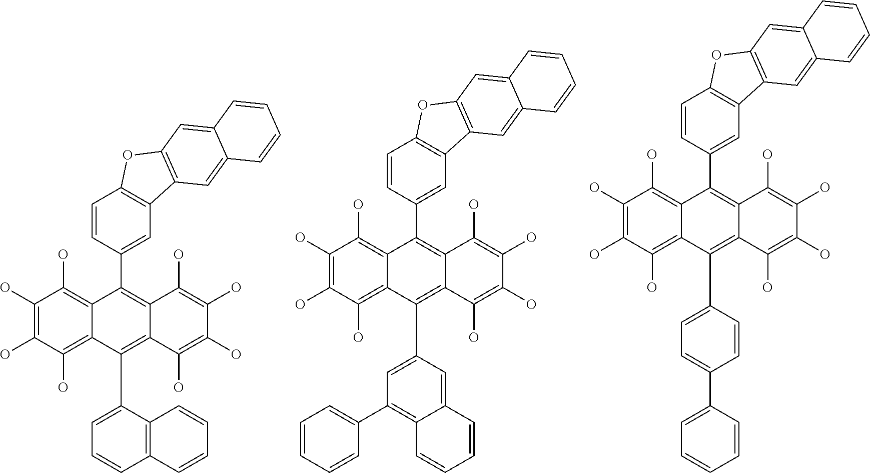 [SMILES: Oc1c(O)c(O)c2c(-c3cccc4ccccc34)c3c(O)c(O)c(O)c(O)c3c(-c3ccc4oc5cc6ccccc6cc5c4c3)c2c1O ; Oc1c(O)c(O)c2c(-c3ccc4oc5cc6ccccc6cc5c4c3)c3c(O)c(O)c(O)c(O)c3c(-c3cc(-c4ccccc4)c4ccccc4c3)c2c1O ; Oc1c(O)c(O)c2c(-c3ccc4oc5cc6ccccc6cc5c4c3)c3c(O)c(O)c(O)c(O)c3c(-c3ccc(-c4ccccc4)cc3)c2c1O]

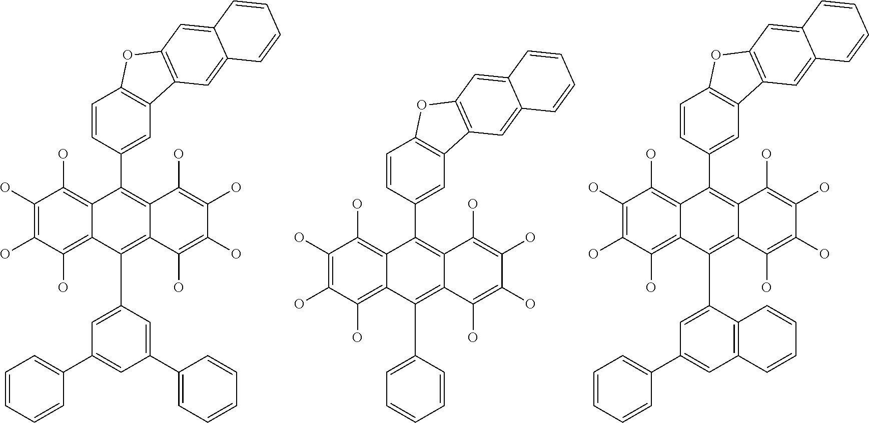 [SMILES: Oc1c(O)c(O)c2c(-c3ccc4oc5cc6ccccc6cc5c4c3)c3c(O)c(O)c(O)c(O)c3c(-c3cc(-c4ccccc4)cc(-c4ccccc4)c3)c2c1O ; Oc1c(O)c(O)c2c(-c3ccc4oc5cc6ccccc6cc5c4c3)c3c(O)c(O)c(O)c(O)c3c(-c3ccccc3)c2c1O ; Oc1c(O)c(O)c2c(-c3cc(-c4ccccc4)cc4ccccc34)c3c(O)c(O)c(O)c(O)c3c(-c3ccc4oc5cc6ccccc6cc5c4c3)c2c1O]

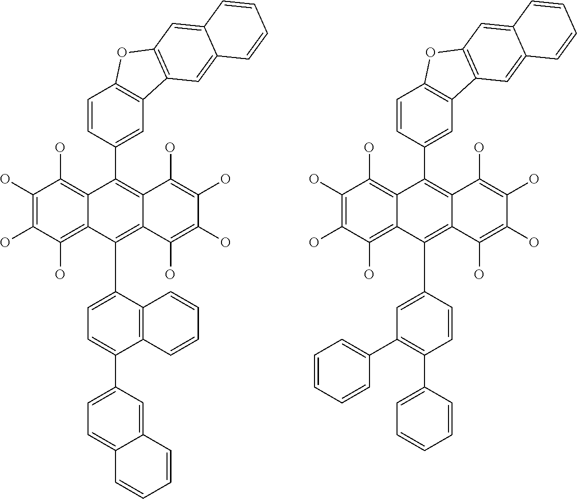 [SMILES: Oc1c(O)c(O)c2c(-c3ccc(-c4ccc5ccccc5c4)c4ccccc34)c3c(O)c(O)c(O)c(O)c3c(-c3ccc4oc5cc6ccccc6cc5c4c3)c2c1O ; Oc1c(O)c(O)c2c(-c3ccc4oc5cc6ccccc6cc5c4c3)c3c(O)c(O)c(O)c(O)c3c(-c3ccc(-c4ccccc4)c(-c4ccccc4)c3)c2c1O]

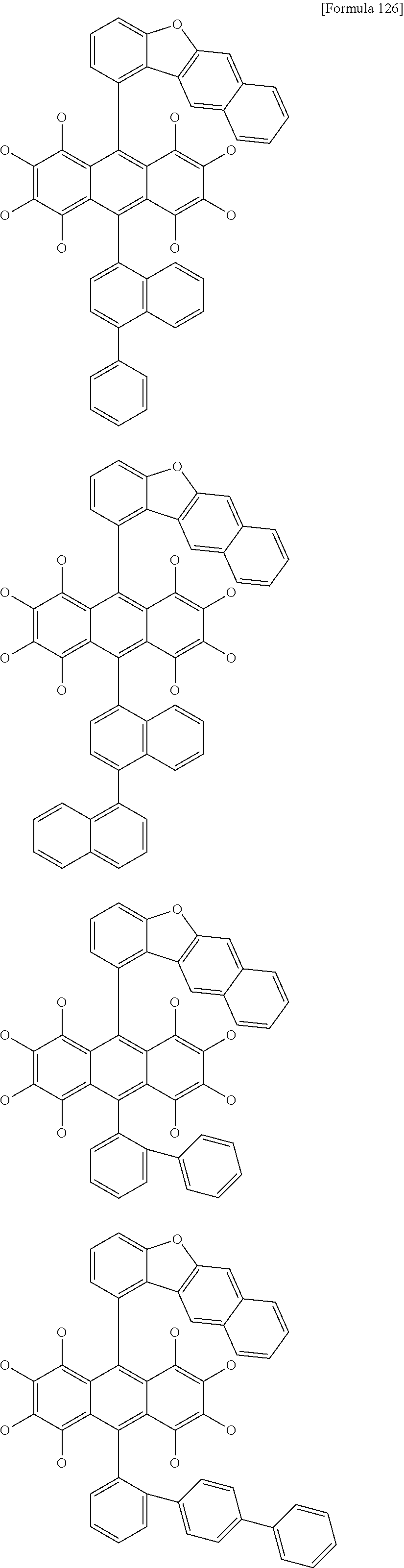

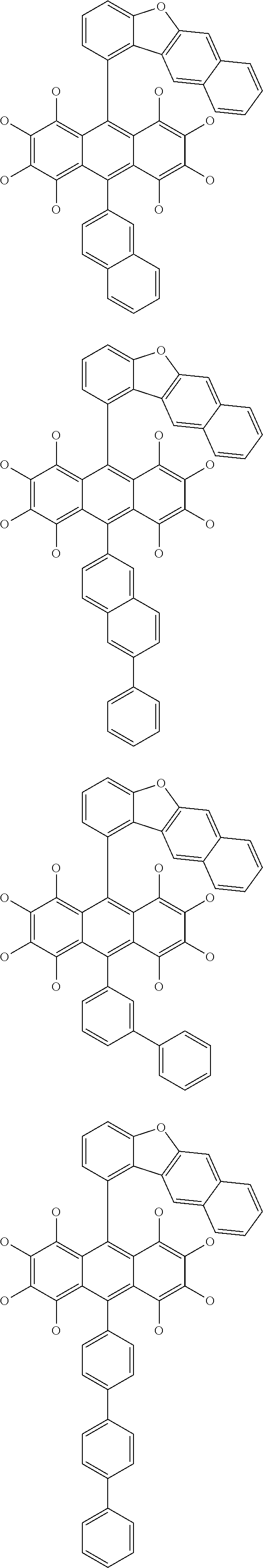

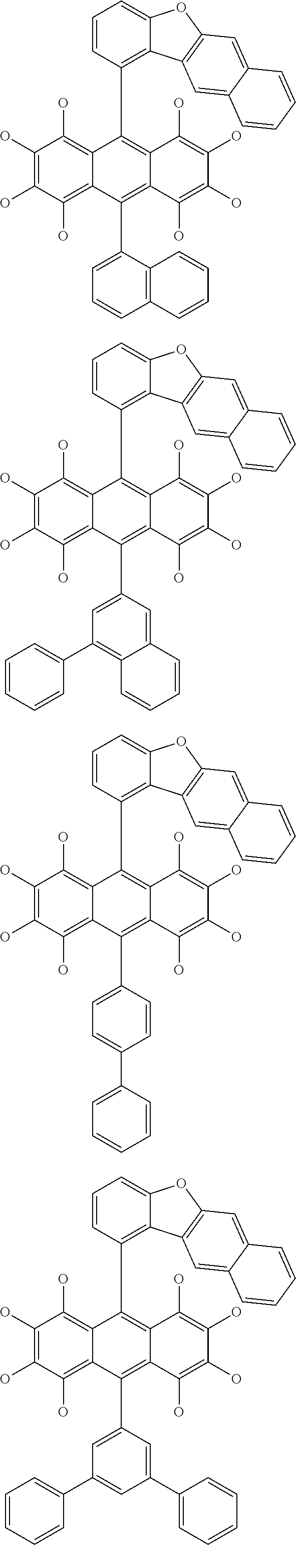

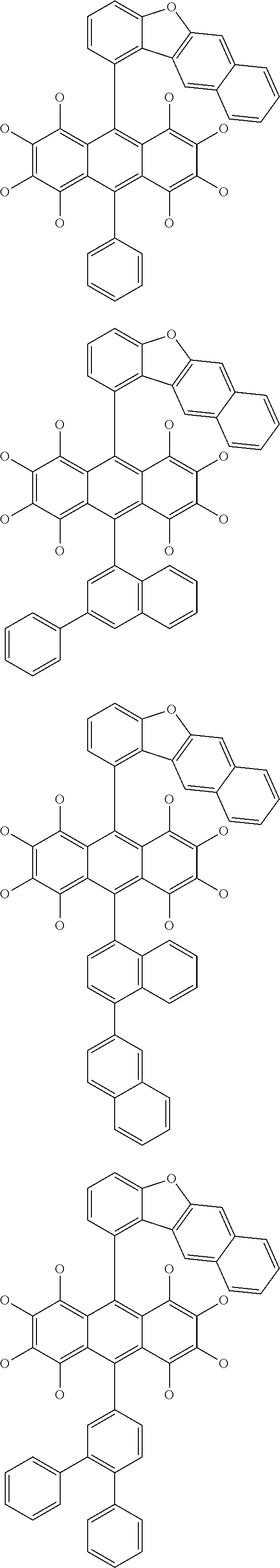

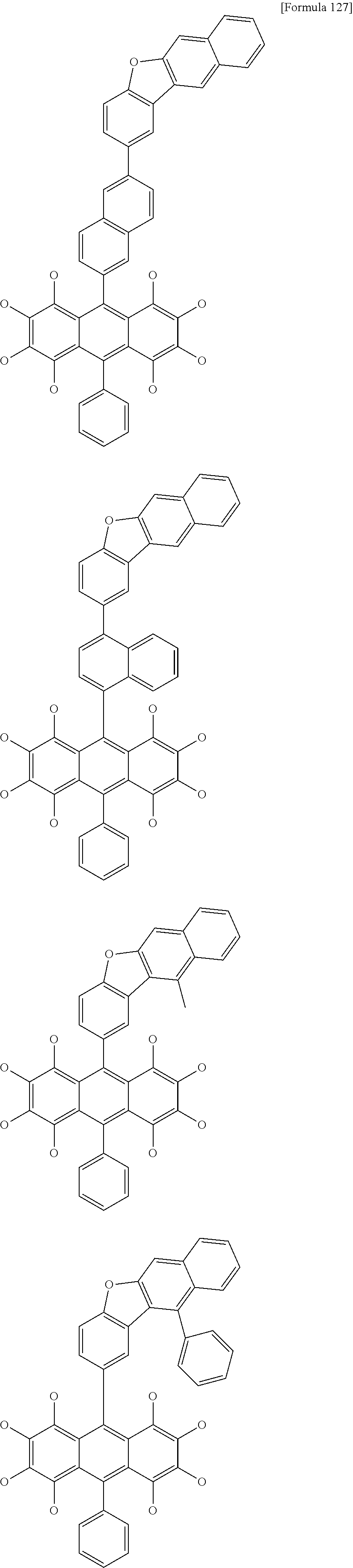 [SMILES: Oc1c(O)c(O)c2c(-c3ccc4cc(-c5ccc6oc7cc8ccccc8cc7c6c5)ccc4c3)c3c(O)c(O)c(O)c(O)c3c(-c3ccccc3)c2c1O ; Oc1c(O)c(O)c2c(-c3ccc(-c4ccc5oc6cc7ccccc7cc6c5c4)c4ccccc34)c3c(O)c(O)c(O)c(O)c3c(-c3ccccc3)c2c1O ; Cc1c2ccccc2cc2oc3ccc(-c4c5c(O)c(O)c(O)c(O)c5c(-c5ccccc5)c5c(O)c(O)c(O)c(O)c45)cc3c12 ; Oc1c(O)c(O)c2c(-c3ccc4oc5cc6ccccc6c(-c6ccccc6)c5c4c3)c3c(O)c(O)c(O)c(O)c3c(-c3ccccc3)c2c1O]

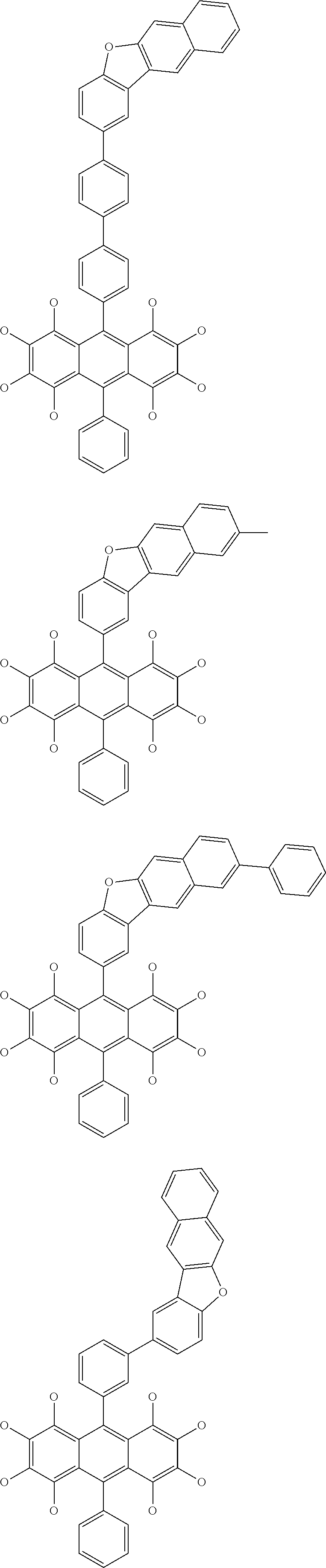 [SMILES: Oc1c(O)c(O)c2c(-c3ccc(-c4ccc(-c5ccc6oc7cc8ccccc8cc7c6c5)cc4)cc3)c3c(O)c(O)c(O)c(O)c3c(-c3ccccc3)c2c1O ; Cc1ccc2cc3oc4ccc(-c5c6c(O)c(O)c(O)c(O)c6c(-c6ccccc6)c6c(O)c(O)c(O)c(O)c56)cc4c3cc2c1 ; Oc1c(O)c(O)c2c(-c3ccc4oc5cc6ccc(-c7ccccc7)cc6cc5c4c3)c3c(O)c(O)c(O)c(O)c3c(-c3ccccc3)c2c1O ; Oc1c(O)c(O)c2c(-c3cccc(-c4ccc5oc6cc7ccccc7cc6c5c4)c3)c3c(O)c(O)c(O)c(O)c3c(-c3ccccc3)c2c1O]

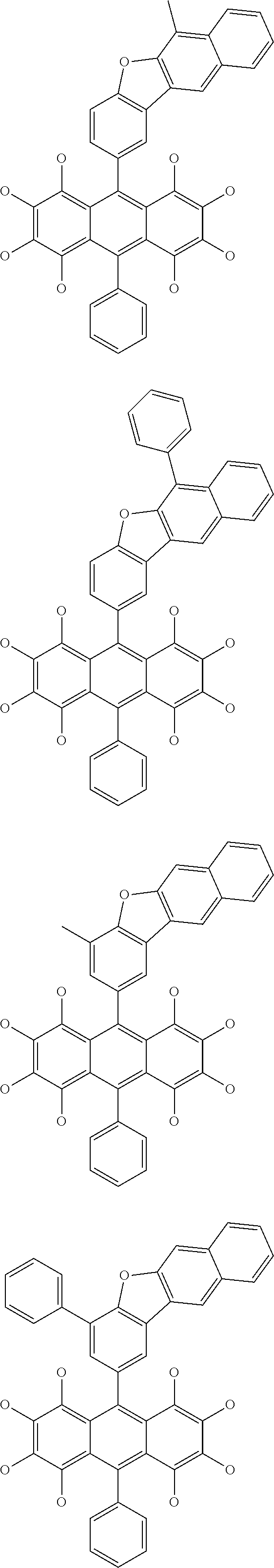 [SMILES: Cc1c2ccccc2cc2c1oc1ccc(-c3c4c(O)c(O)c(O)c(O)c4c(-c4ccccc4)c4c(O)c(O)c(O)c(O)c34)cc12 ; Oc1c(O)c(O)c2c(-c3ccc4oc5c(-c6ccccc6)c6ccccc6cc5c4c3)c3c(O)c(O)c(O)c(O)c3c(-c3ccccc3)c2c1O ; Cc1cc(-c2c3c(O)c(O)c(O)c(O)c3c(-c3ccccc3)c3c(O)c(O)c(O)c(O)c23)cc2c1oc1cc3ccccc3cc12 ; Oc1c(O)c(O)c2c(-c3cc(-c4ccccc4)c4oc5cc6ccccc6cc5c4c3)c3c(O)c(O)c(O)c(O)c3c(-c3ccccc3)c2c1O]

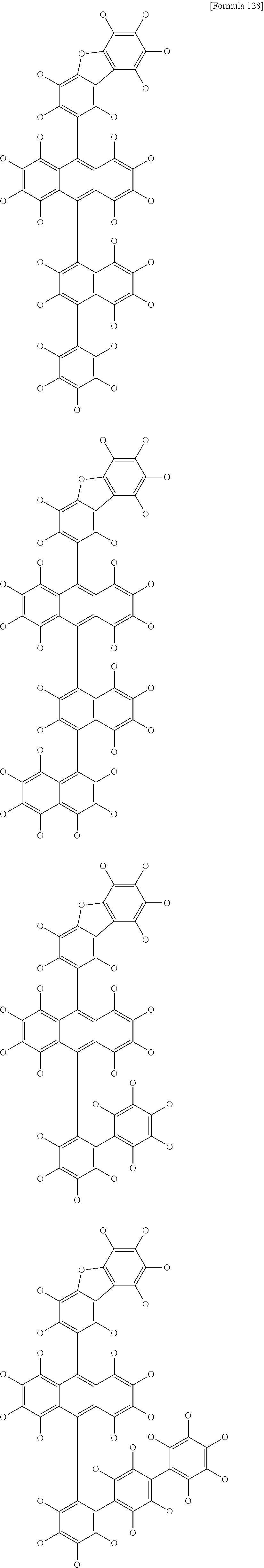

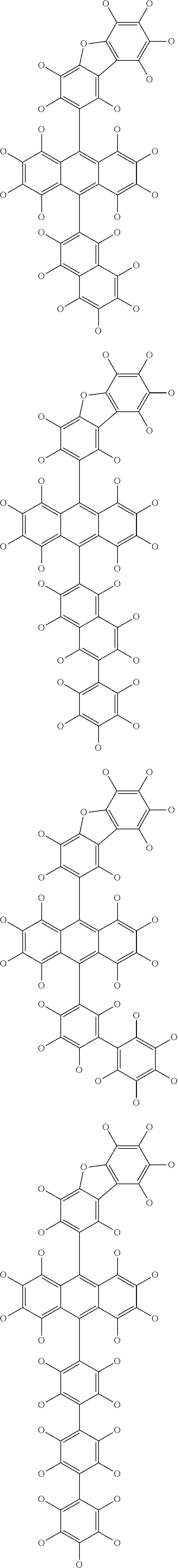

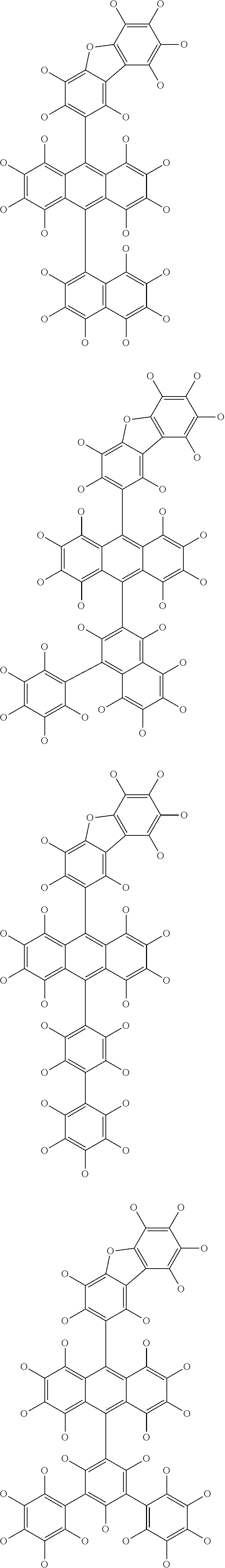

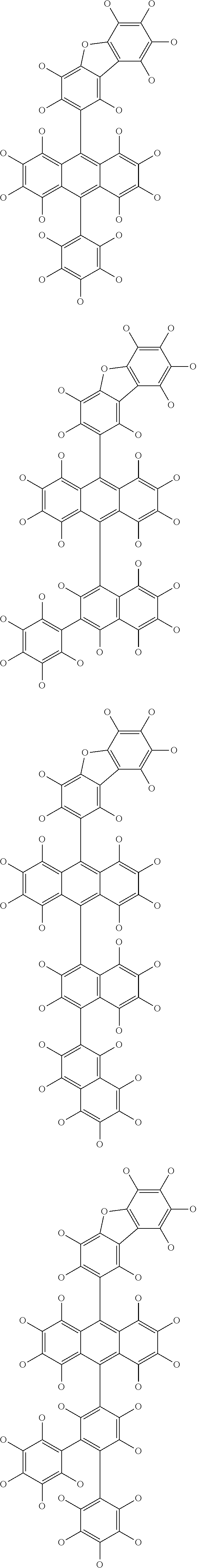

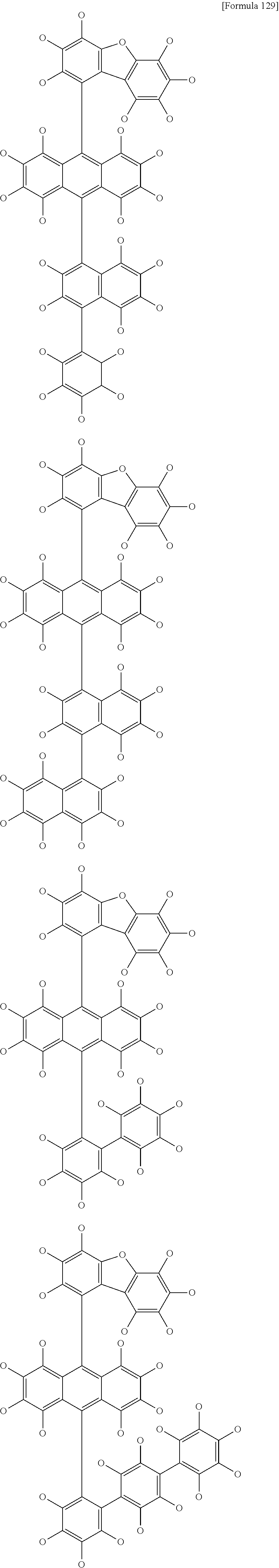

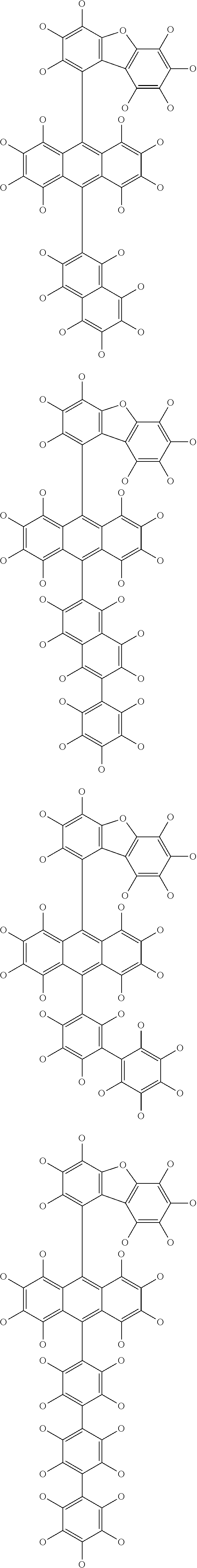

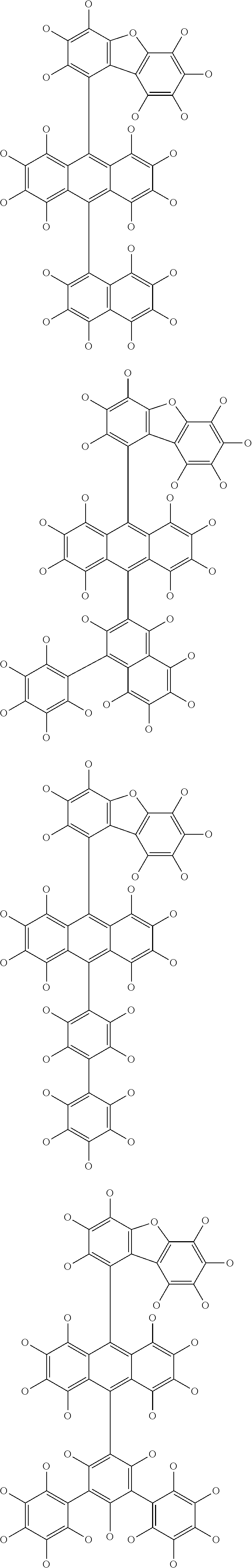

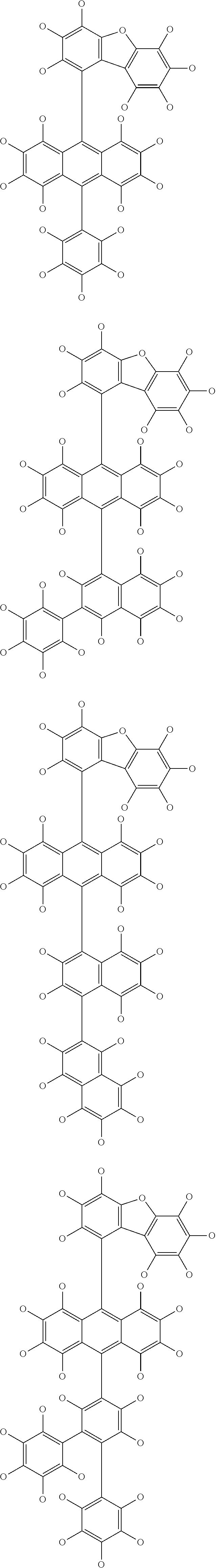

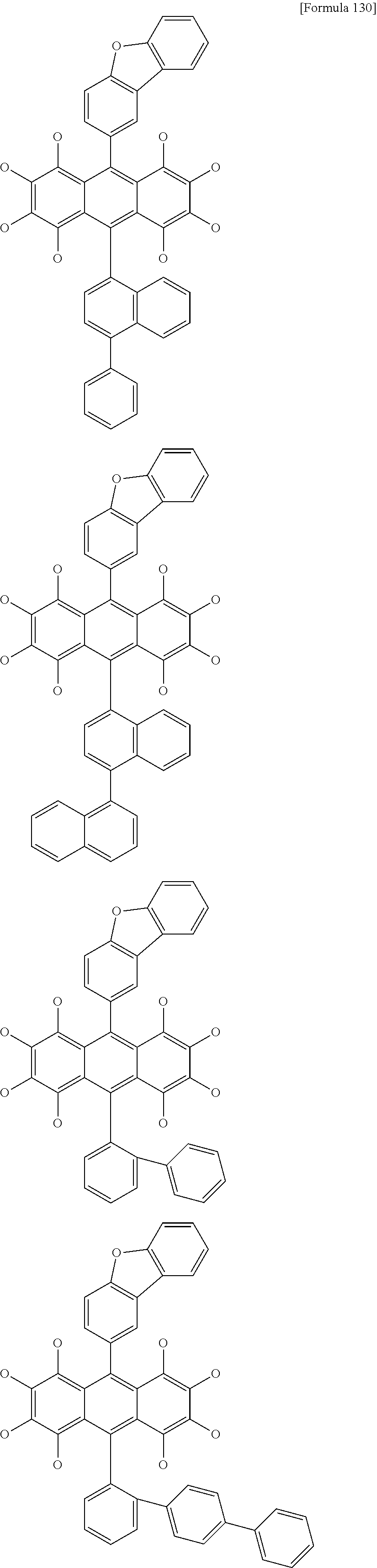

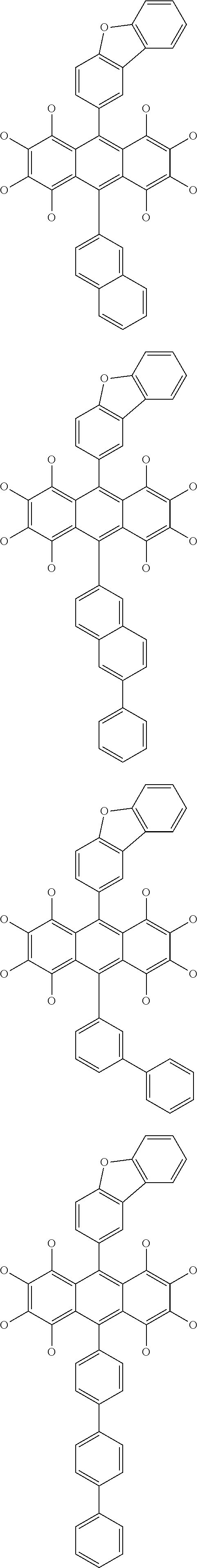

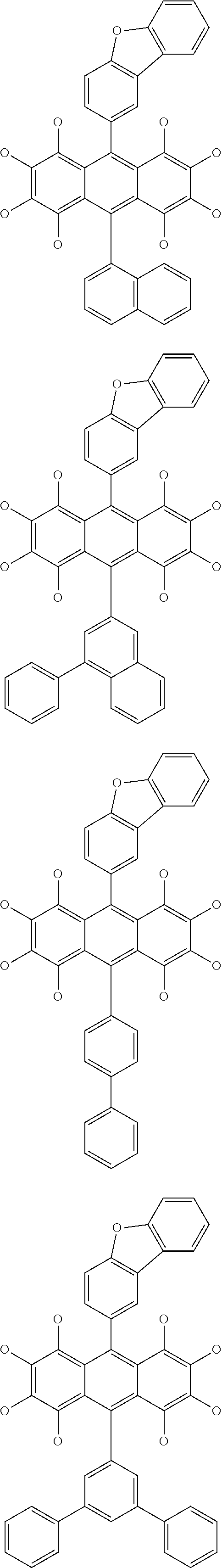

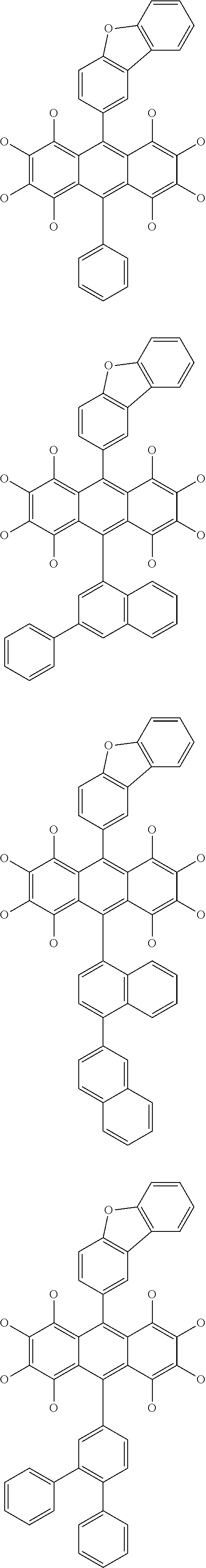 [SMILES: Oc1c(O)c(O)c2c(-c3ccc4oc5ccccc5c4c3)c3c(O)c(O)c(O)c(O)c3c(-c3ccccc3)c2c1O ; Oc1c(O)c(O)c2c(-c3cc(-c4ccccc4)cc4ccccc34)c3c(O)c(O)c(O)c(O)c3c(-c3ccc4oc5ccccc5c4c3)c2c1O ; Oc1c(O)c(O)c2c(-c3ccc(-c4ccc5ccccc5c4)c4ccccc34)c3c(O)c(O)c(O)c(O)c3c(-c3ccc4oc5ccccc5c4c3)c2c1O ; Oc1c(O)c(O)c2c(-c3ccc4oc5ccccc5c4c3)c3c(O)c(O)c(O)c(O)c3c(-c3ccc(-c4ccccc4)c(-c4ccccc4)c3)c2c1O]

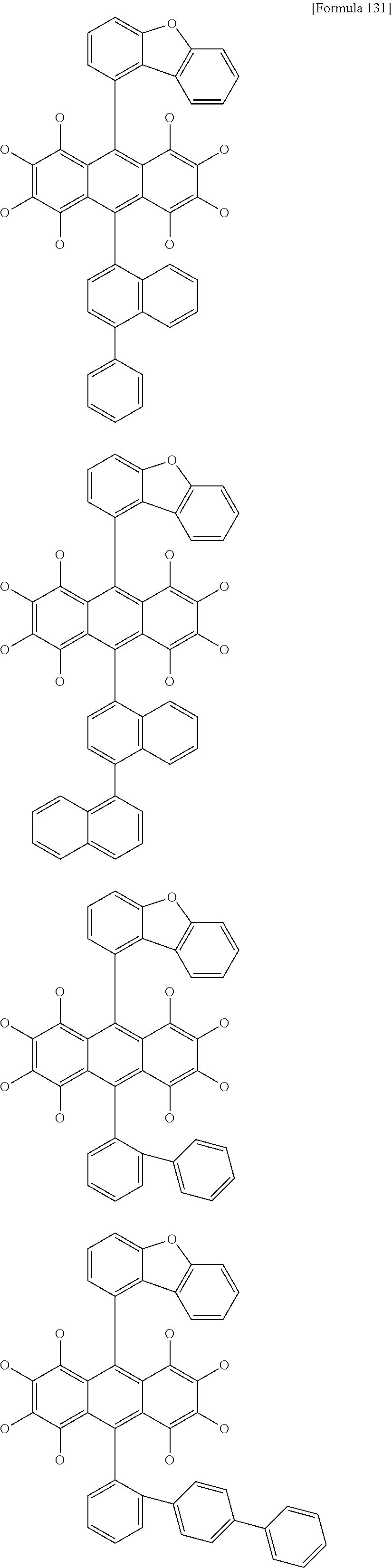

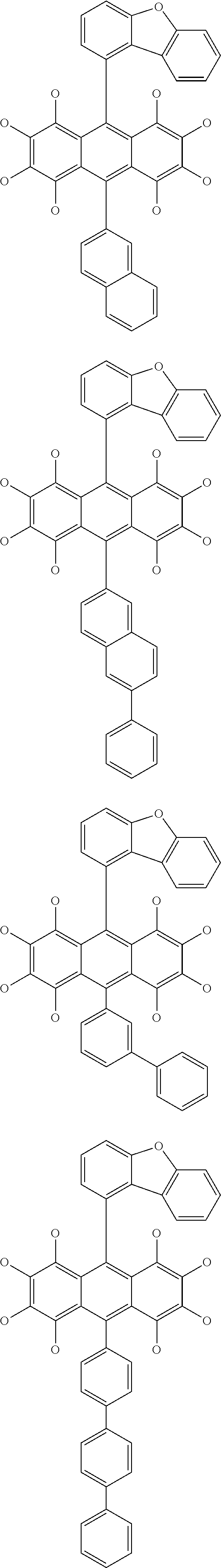

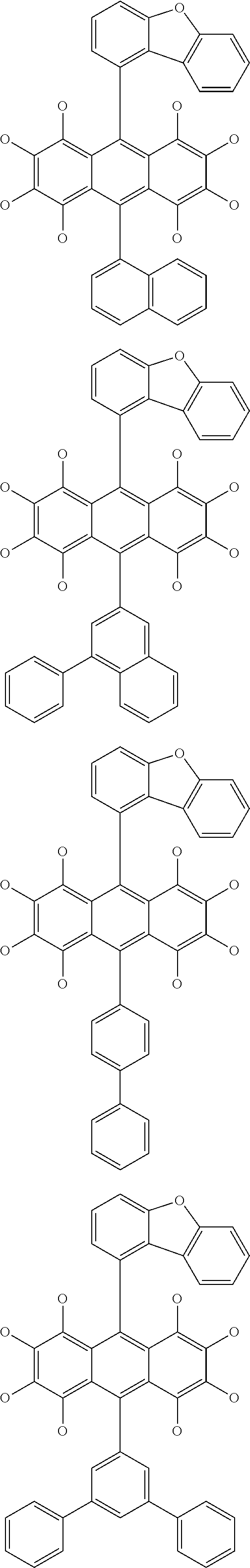

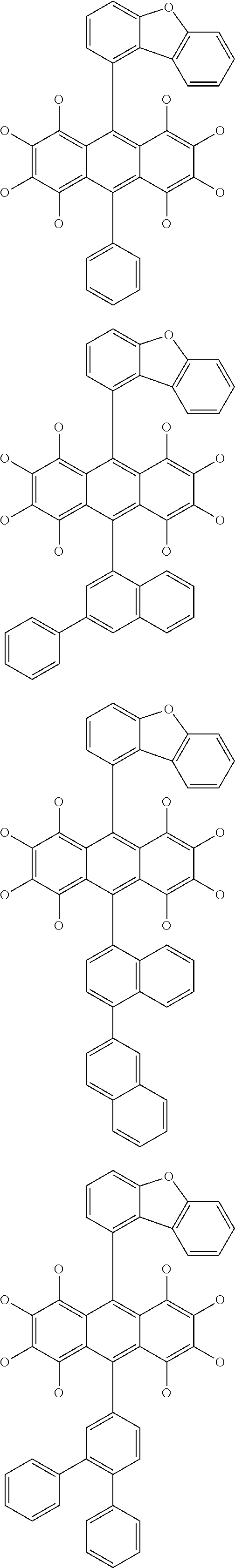

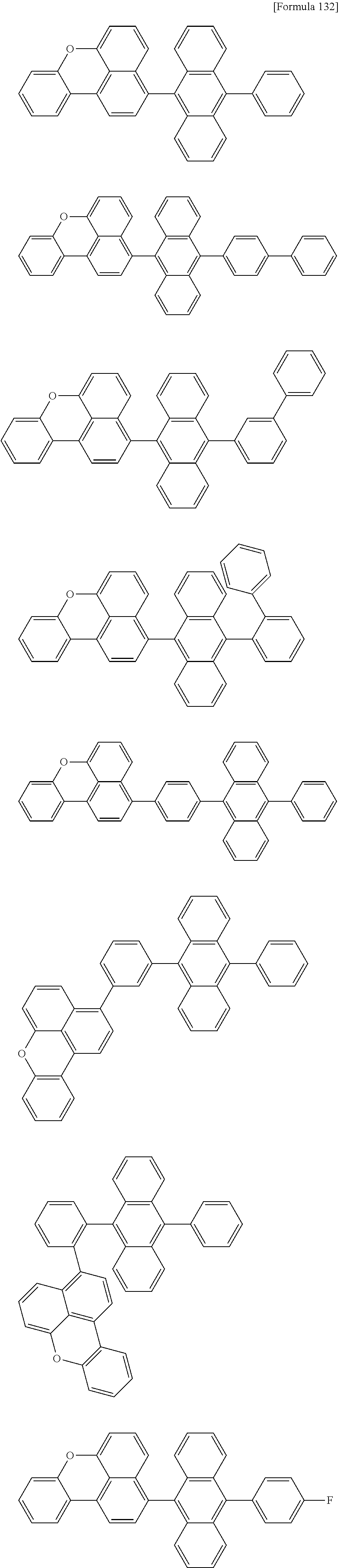

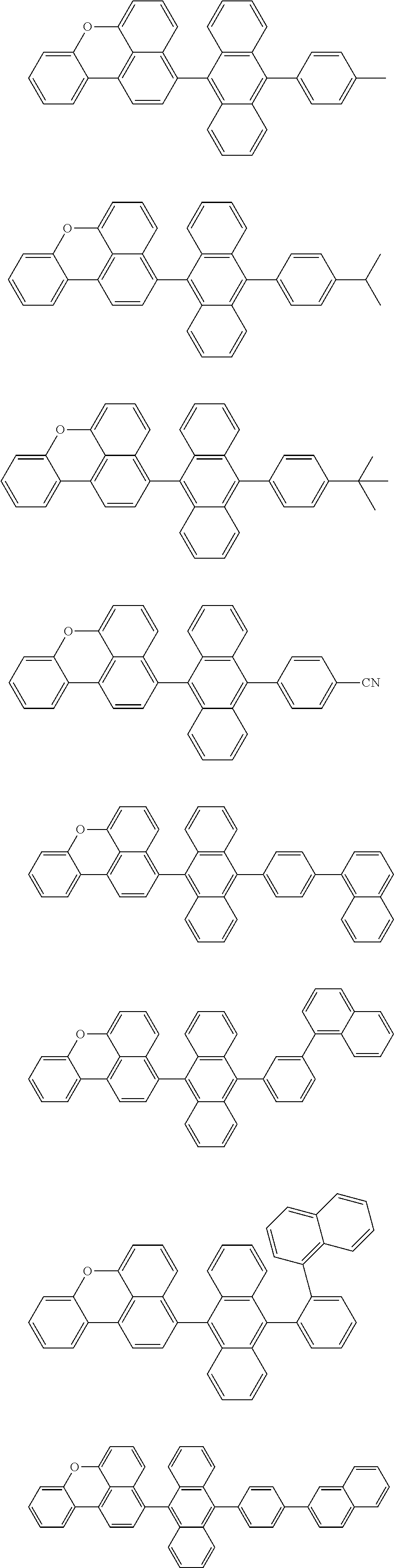

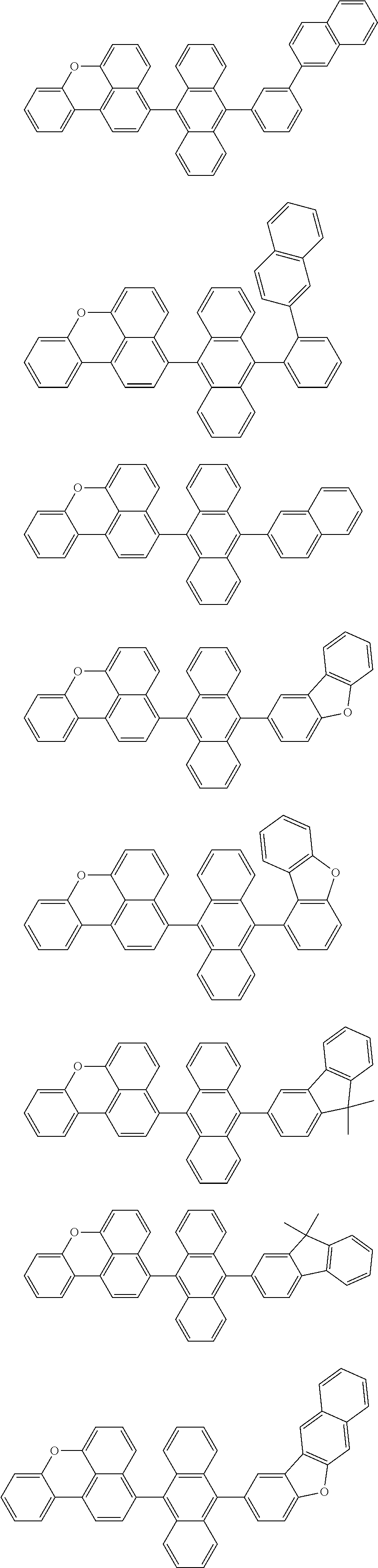

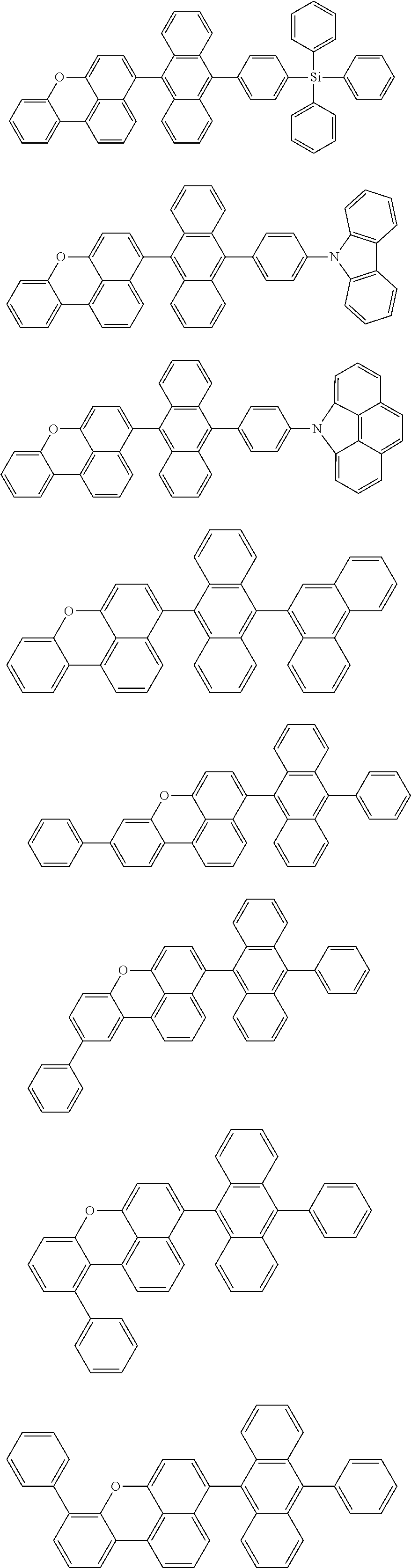

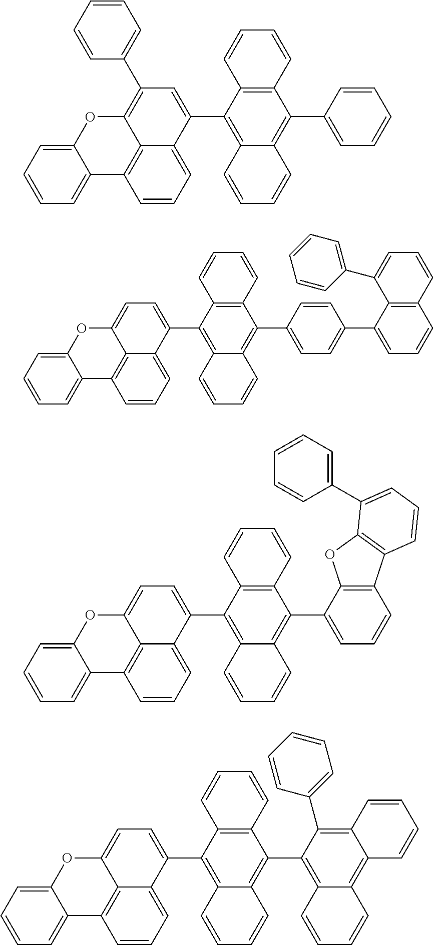

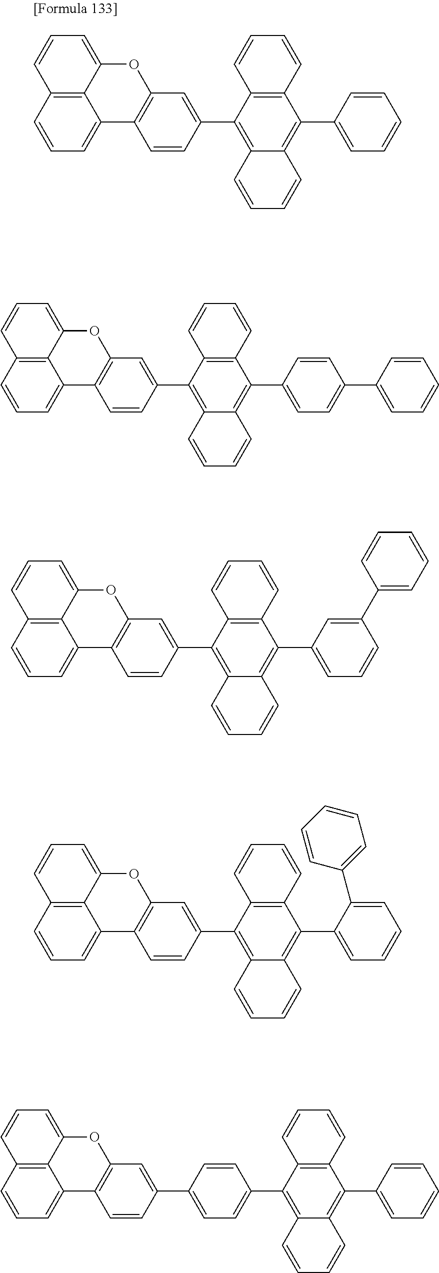 [SMILES: c1ccc(-c2c3ccccc3c(-c3ccc4c(c3)Oc3cccc5cccc-4c35)c3ccccc23)cc1 ; c1ccc(-c2ccc(-c3c4ccccc4c(-c4ccc5c(c4)Oc4cccc6cccc-5c46)c4ccccc34)cc2)cc1 ; c1ccc(-c2cccc(-c3c4ccccc4c(-c4ccc5c(c4)Oc4cccc6cccc-5c46)c4ccccc34)c2)cc1 ; c1ccc(-c2ccccc2-c2c3ccccc3c(-c3ccc4c(c3)Oc3cccc5cccc-4c35)c3ccccc23)cc1 ; c1ccc(-c2c3ccccc3c(-c3ccc(-c4ccc5c(c4)Oc4cccc6cccc-5c46)cc3)c3ccccc23)cc1]

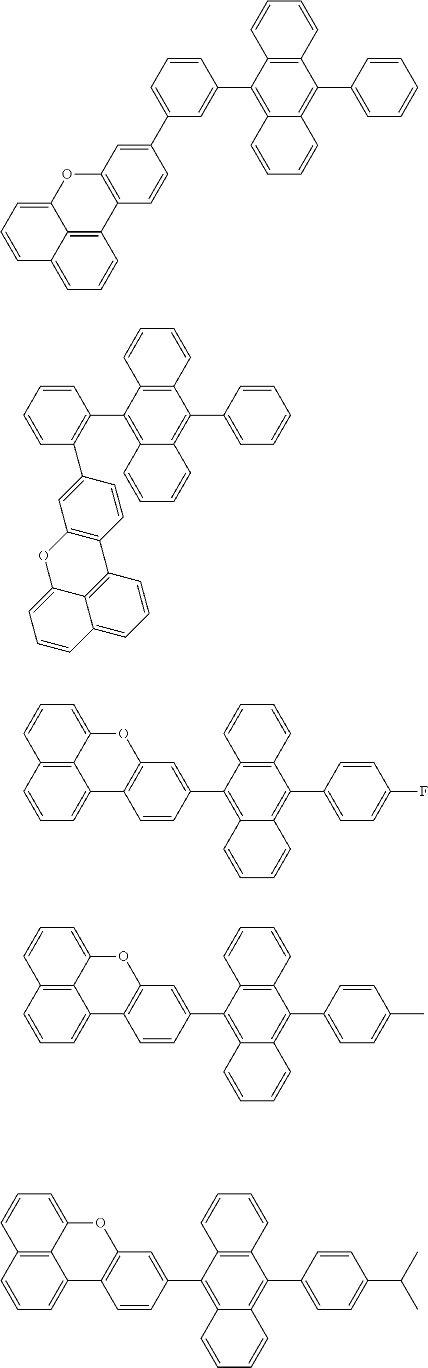 [SMILES: c1ccc(-c2c3ccccc3c(-c3cccc(-c4ccc5c(c4)Oc4cccc6cccc-5c46)c3)c3ccccc23)cc1 ; c1ccc(-c2c3ccccc3c(-c3ccccc3-c3ccc4c(c3)Oc3cccc5cccc-4c35)c3ccccc23)cc1 ; Fc1ccc(-c2c3ccccc3c(-c3ccc4c(c3)Oc3cccc5cccc-4c35)c3ccccc23)cc1 ; Cc1ccc(-c2c3ccccc3c(-c3ccc4c(c3)Oc3cccc5cccc-4c35)c3ccccc23)cc1 ; CC(C)c1ccc(-c2c3ccccc3c(-c3ccc4c(c3)Oc3cccc5cccc-4c35)c3ccccc23)cc1]

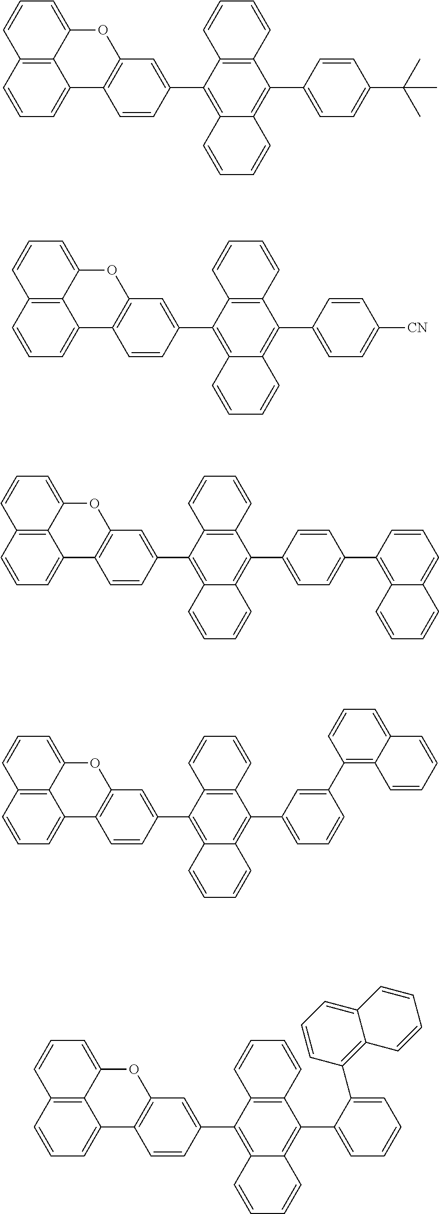 [SMILES: CC(C)(C)c1ccc(-c2c3ccccc3c(-c3ccc4c(c3)Oc3cccc5cccc-4c35)c3ccccc23)cc1 ; N#Cc1ccc(-c2c3ccccc3c(-c3ccc4c(c3)Oc3cccc5cccc-4c35)c3ccccc23)cc1 ; c1ccc2c(-c3ccc(-c4c5ccccc5c(-c5ccc6c(c5)Oc5cccc7cccc-6c57)c5ccccc45)cc3)cccc2c1 ; c1cc(-c2cccc3ccccc23)cc(-c2c3ccccc3c(-c3ccc4c(c3)Oc3cccc5cccc-4c35)c3ccccc23)c1 ; c1ccc(-c2c3ccccc3c(-c3ccc4c(c3)Oc3cccc5cccc-4c35)c3ccccc23)c(-c2cccc3ccccc23)c1]

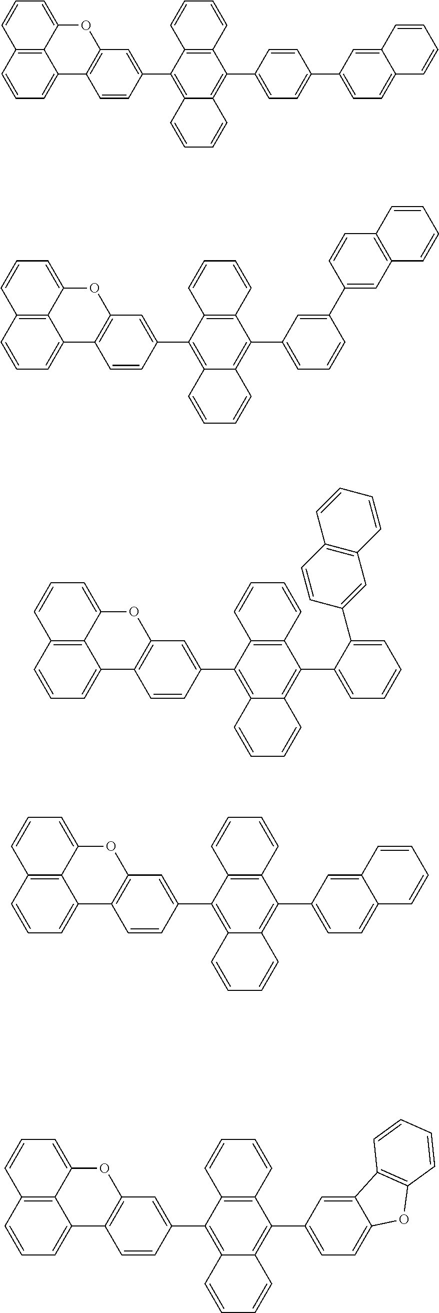 [SMILES: c1ccc2cc(-c3ccc(-c4c5ccccc5c(-c5ccc6c(c5)Oc5cccc7cccc-6c57)c5ccccc45)cc3)ccc2c1 ; c1cc(-c2ccc3ccccc3c2)cc(-c2c3ccccc3c(-c3ccc4c(c3)Oc3cccc5cccc-4c35)c3ccccc23)c1 ; c1ccc(-c2c3ccccc3c(-c3ccc4c(c3)Oc3cccc5cccc-4c35)c3ccccc23)c(-c2ccc3ccccc3c2)c1 ; c1ccc2cc(-c3c4ccccc4c(-c4ccc5c(c4)Oc4cccc6cccc-5c46)c4ccccc34)ccc2c1 ; c1cc2c3c(cccc3c1)-c1ccc(-c3c4ccccc4c(-c4ccc5oc6ccccc6c5c4)c4ccccc34)cc1O2]

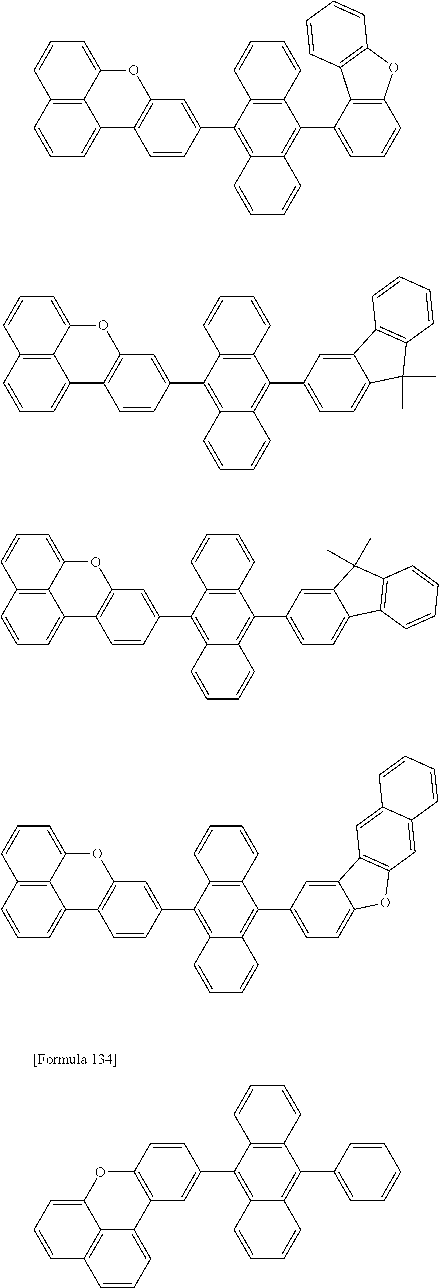 [SMILES: c1cc2c3c(cccc3c1)-c1ccc(-c3c4ccccc4c(-c4cccc5oc6ccccc6c45)c4ccccc34)cc1O2 ; CC1(C)c2ccccc2-c2cc(-c3c4ccccc4c(-c4ccc5c(c4)Oc4cccc6cccc-5c46)c4ccccc34)ccc21 ; CC1(C)c2ccccc2-c2ccc(-c3c4ccccc4c(-c4ccc5c(c4)Oc4cccc6cccc-5c46)c4ccccc34)cc21 ; c1ccc2cc3c(cc2c1)oc1ccc(-c2c4ccccc4c(-c4ccc5c(c4)Oc4cccc6cccc-5c46)c4ccccc24)cc13 ; c1ccc(-c2c3ccccc3c(-c3ccc4c(c3)-c3cccc5cccc(c35)O4)c3ccccc23)cc1]

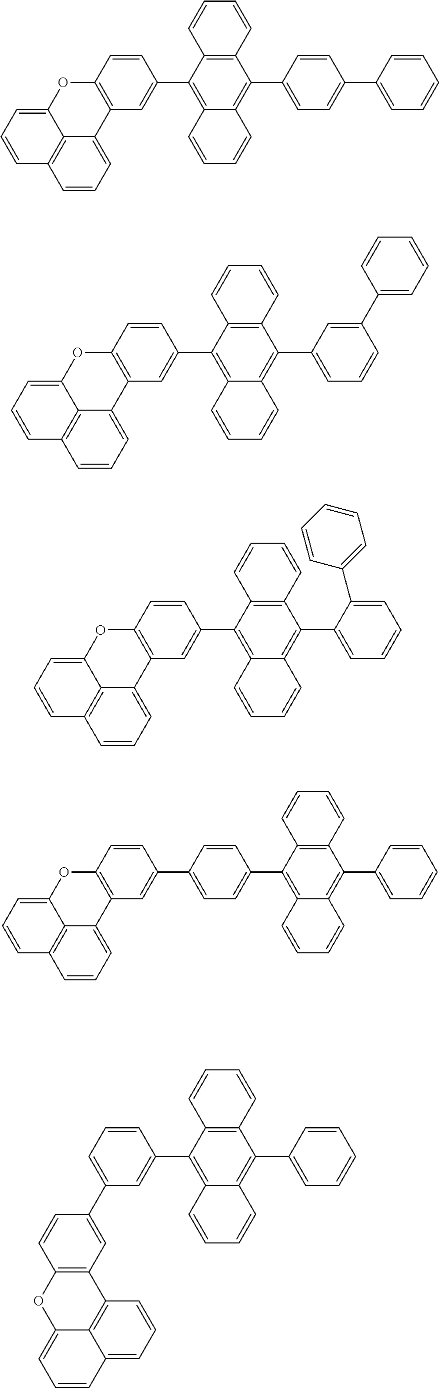 [SMILES: c1ccc(-c2ccc(-c3c4ccccc4c(-c4ccc5c(c4)-c4cccc6cccc(c46)O5)c4ccccc34)cc2)cc1 ; c1ccc(-c2cccc(-c3c4ccccc4c(-c4ccc5c(c4)-c4cccc6cccc(c46)O5)c4ccccc34)c2)cc1 ; c1ccc(-c2ccccc2-c2c3ccccc3c(-c3ccc4c(c3)-c3cccc5cccc(c35)O4)c3ccccc23)cc1 ; c1ccc(-c2c3ccccc3c(-c3ccc(-c4ccc5c(c4)-c4cccc6cccc(c46)O5)cc3)c3ccccc23)cc1 ; c1ccc(-c2c3ccccc3c(-c3cccc(-c4ccc5c(c4)-c4cccc6cccc(c46)O5)c3)c3ccccc23)cc1]

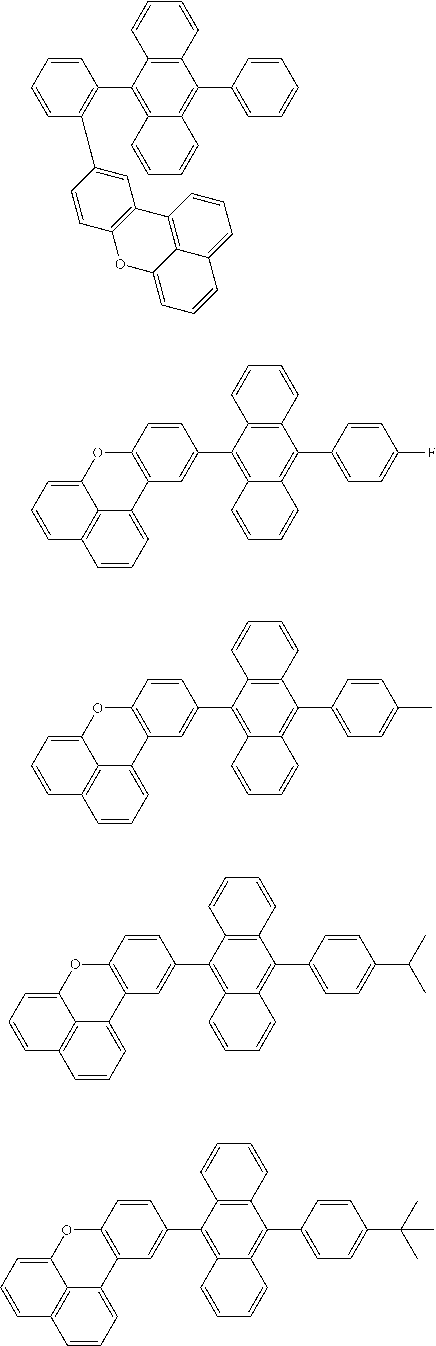 [SMILES: c1ccc(-c2c3ccccc3c(-c3ccccc3-c3ccc4c(c3)-c3cccc5cccc(c35)O4)c3ccccc23)cc1 ; Fc1ccc(-c2c3ccccc3c(-c3ccc4c(c3)-c3cccc5cccc(c35)O4)c3ccccc23)cc1 ; Cc1ccc(-c2c3ccccc3c(-c3ccc4c(c3)-c3cccc5cccc(c35)O4)c3ccccc23)cc1 ; CC(C)c1ccc(-c2c3ccccc3c(-c3ccc4c(c3)-c3cccc5cccc(c35)O4)c3ccccc23)cc1 ; CC(C)(C)c1ccc(-c2c3ccccc3c(-c3ccc4c(c3)-c3cccc5cccc(c35)O4)c3ccccc23)cc1]

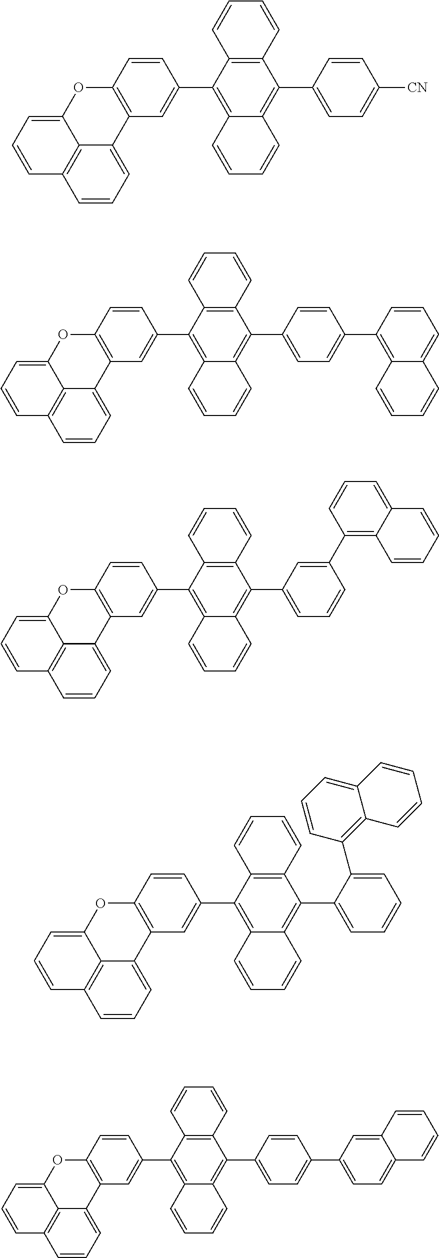 [SMILES: N#Cc1ccc(-c2c3ccccc3c(-c3ccc4c(c3)-c3cccc5cccc(c35)O4)c3ccccc23)cc1 ; c1ccc2c(-c3ccc(-c4c5ccccc5c(-c5ccc6c(c5)-c5cccc7cccc(c57)O6)c5ccccc45)cc3)cccc2c1 ; c1cc(-c2cccc3ccccc23)cc(-c2c3ccccc3c(-c3ccc4c(c3)-c3cccc5cccc(c35)O4)c3ccccc23)c1 ; c1ccc(-c2c3ccccc3c(-c3ccc4c(c3)-c3cccc5cccc(c35)O4)c3ccccc23)c(-c2cccc3ccccc23)c1 ; c1ccc2cc(-c3ccc(-c4c5ccccc5c(-c5ccc6c(c5)-c5cccc7cccc(c57)O6)c5ccccc45)cc3)ccc2c1]

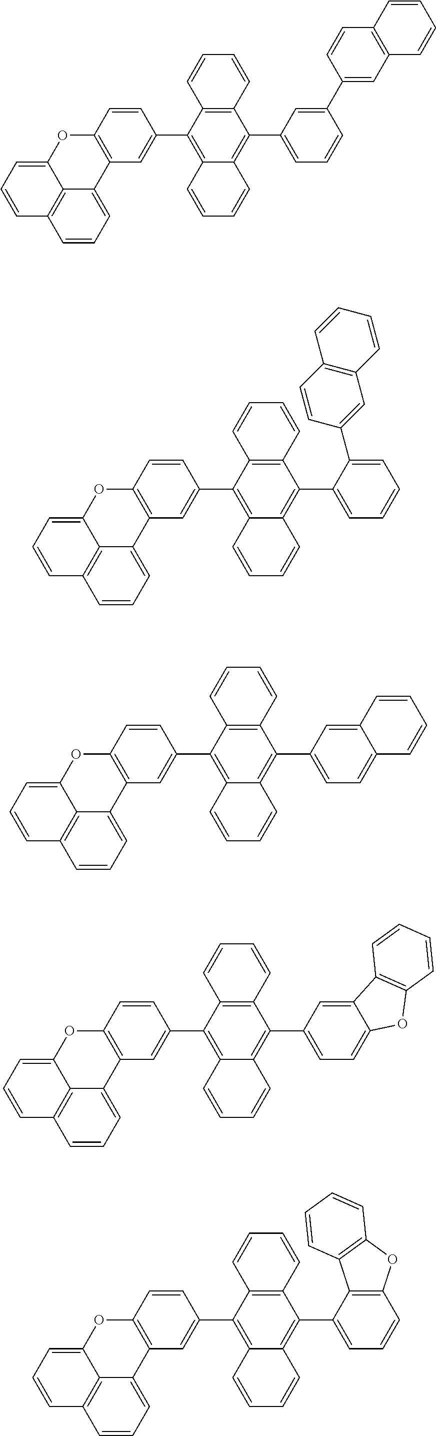 [SMILES: c1cc(-c2ccc3ccccc3c2)cc(-c2c3ccccc3c(-c3ccc4c(c3)-c3cccc5cccc(c35)O4)c3ccccc23)c1 ; c1ccc(-c2c3ccccc3c(-c3ccc4c(c3)-c3cccc5cccc(c35)O4)c3ccccc23)c(-c2ccc3ccccc3c2)c1 ; c1ccc2cc(-c3c4ccccc4c(-c4ccc5c(c4)-c4cccc6cccc(c46)O5)c4ccccc34)ccc2c1 ; c1cc2c3c(cccc3c1)-c1cc(-c3c4ccccc4c(-c4ccc5oc6ccccc6c5c4)c4ccccc34)ccc1O2 ; c1cc2c3c(cccc3c1)-c1cc(-c3c4ccccc4c(-c4cccc5oc6ccccc6c45)c4ccccc34)ccc1O2]

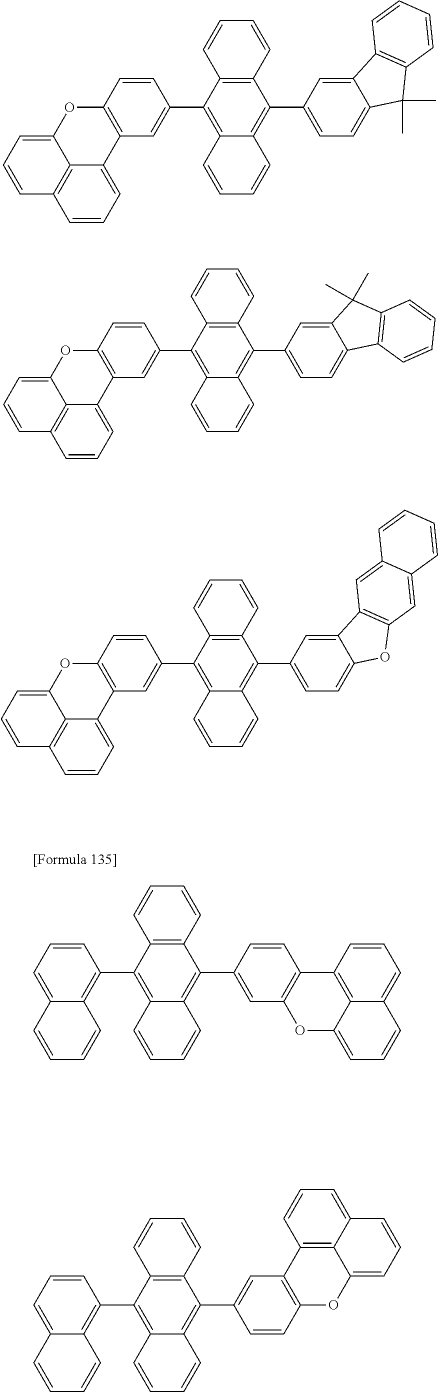 [SMILES: CC1(C)c2ccccc2-c2cc(-c3c4ccccc4c(-c4ccc5c(c4)-c4cccc6cccc(c46)O5)c4ccccc34)ccc21 ; CC1(C)c2ccccc2-c2ccc(-c3c4ccccc4c(-c4ccc5c(c4)-c4cccc6cccc(c46)O5)c4ccccc34)cc21 ; c1ccc2cc3c(cc2c1)oc1ccc(-c2c4ccccc4c(-c4ccc5c(c4)-c4cccc6cccc(c46)O5)c4ccccc24)cc13 ; c1ccc2c(-c3c4ccccc4c(-c4ccc5c(c4)Oc4cccc6cccc-5c46)c4ccccc34)cccc2c1 ; c1ccc2c(-c3c4ccccc4c(-c4ccc5c(c4)-c4cccc6cccc(c46)O5)c4ccccc34)cccc2c1]

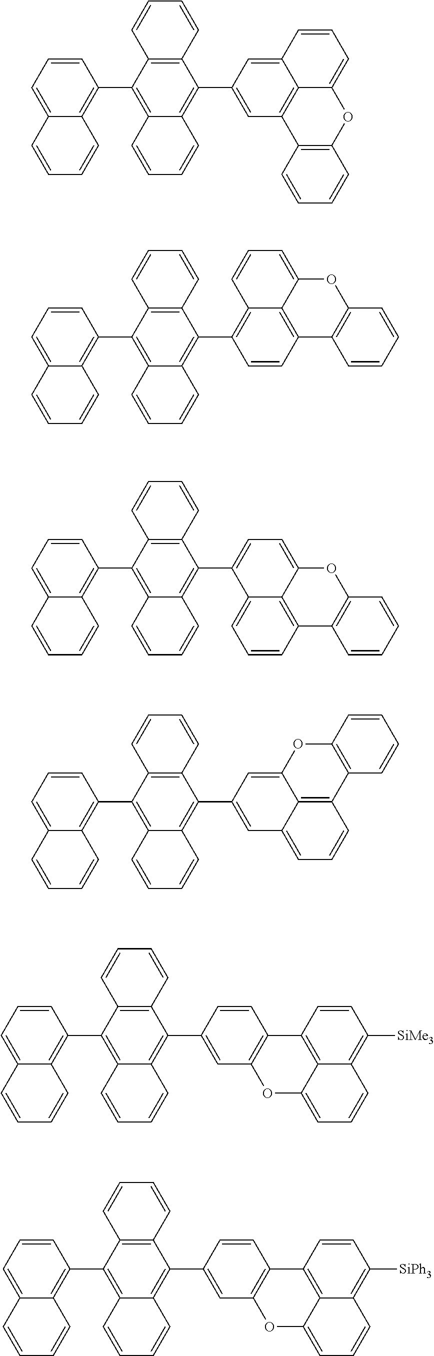

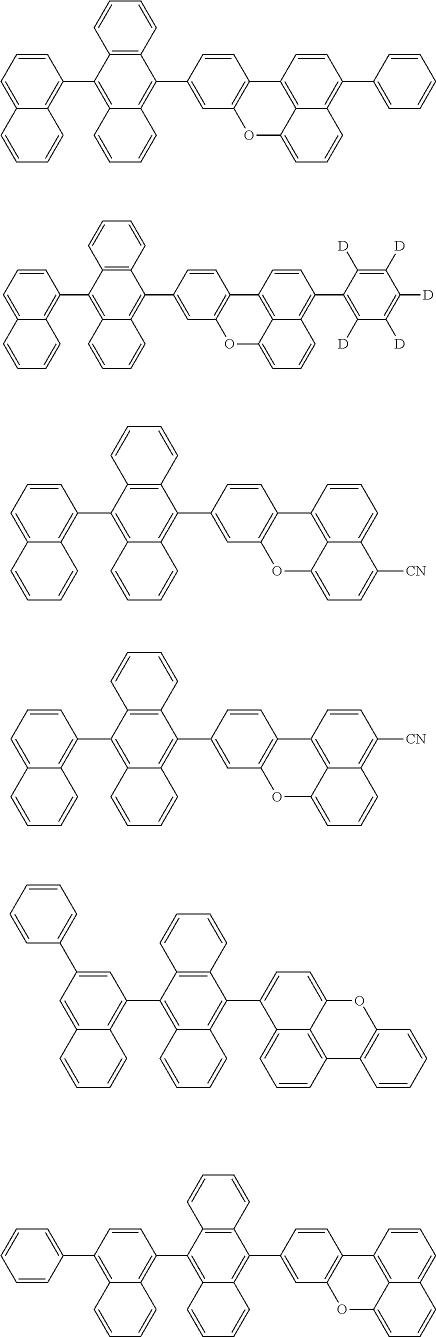 [SMILES: c1ccc(-c2ccc3c4c(cccc24)Oc2cc(-c4c5ccccc5c(-c5cccc6ccccc56)c5ccccc45)ccc2-3)cc1 ; [2H]c1c([2H])c([2H])c(-c2ccc3c4c(cccc24)Oc2cc(-c4c5ccccc5c(-c5cccc6ccccc56)c5ccccc45)ccc2-3)c([2H])c1[2H] ; N#Cc1ccc2c3c(cccc13)-c1ccc(-c3c4ccccc4c(-c4cccc5ccccc45)c4ccccc34)cc1O2 ; N#Cc1ccc2c3c(cccc13)Oc1cc(-c3c4ccccc4c(-c4cccc5ccccc45)c4ccccc34)ccc1-2 ; c1ccc(-c2cc(-c3c4ccccc4c(-c4ccc5c6c(cccc46)-c4ccccc4O5)c4ccccc34)c3ccccc3c2)cc1 ; c1ccc(-c2ccc(-c3c4ccccc4c(-c4ccc5c(c4)Oc4cccc6cccc-5c46)c4ccccc34)c3ccccc23)cc1]

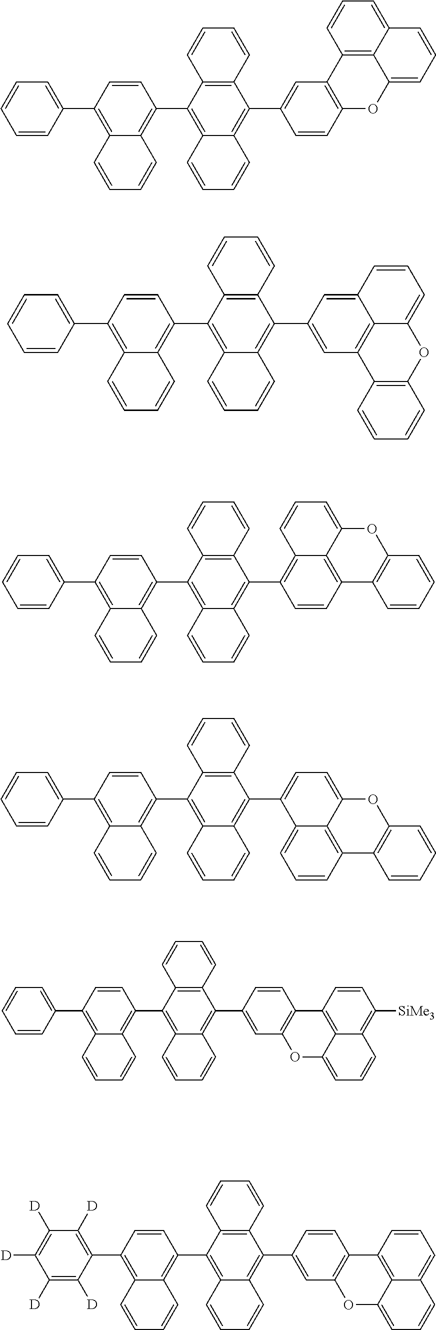

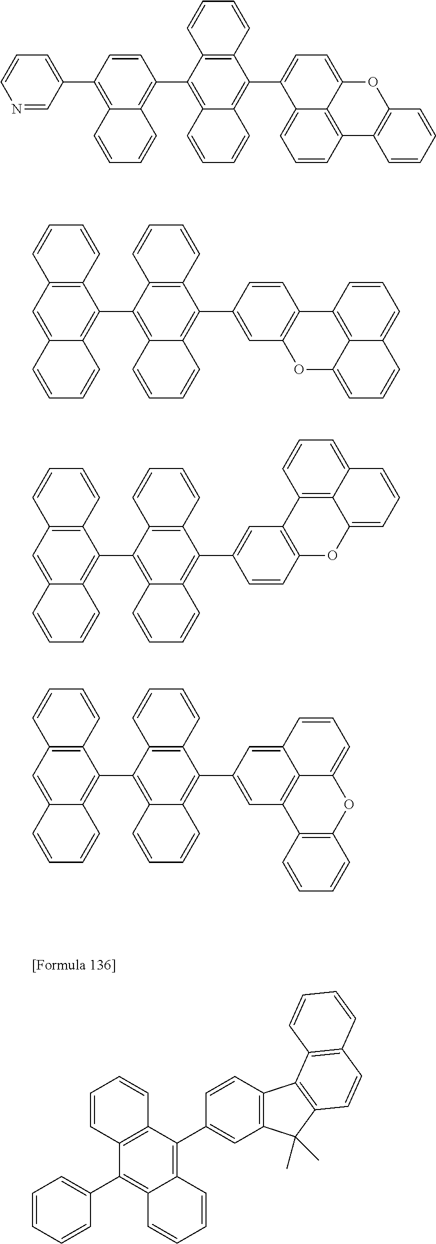

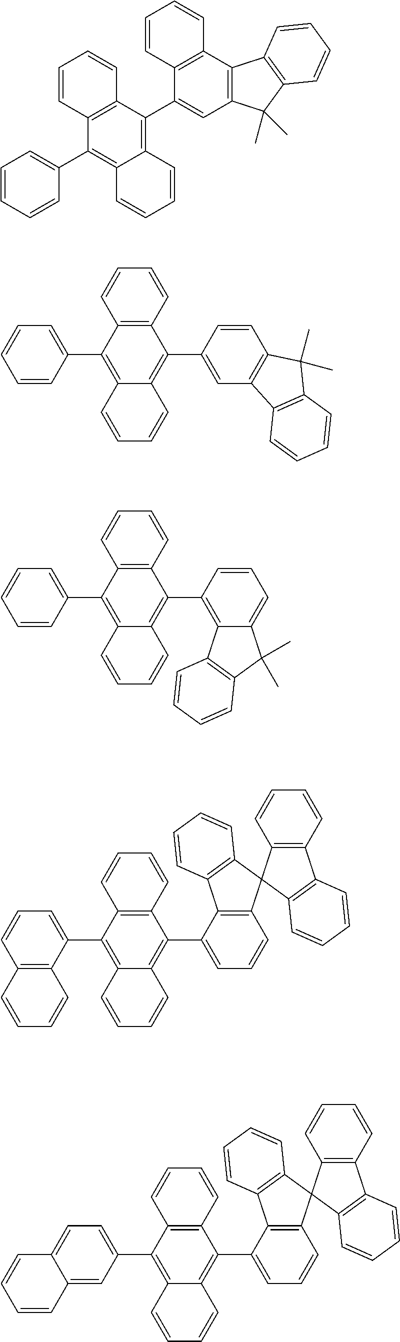

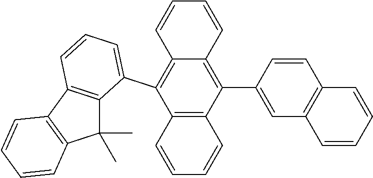 [SMILES: CC1(C)c2ccccc2-c2cccc(-c3c4ccccc4c(-c4ccc5ccccc5c4)c4ccccc34)c21]

### Third Compound and Fourth Compound

In the organic EL device according to the exemplary embodiment, it is also preferable that the first emitting layer further contains a third compound that emits fluorescence.

In the organic EL device according to the exemplary embodiment, it is also preferable that the second emitting layer further contains a fourth compound that emits fluorescence.

When the first emitting layer contains the third compound and the second emitting layer contains the fourth compound, the third compound and the fourth compound are mutually the same or different.

The third compound and the fourth compound are each independently at least one compound selected from the group consisting of a compound represented by a formula (3), a compound represented by a formula (4), a compound represented by a formula (5), a compound represented by a formula (6), a compound represented by a formula (7), a compound represented by a formula (8), a compound represented by a formula (9), and a compound represented by a formula (10).

### Compound Represented by Formula (3)

The compound represented by the formula (3) will be described.

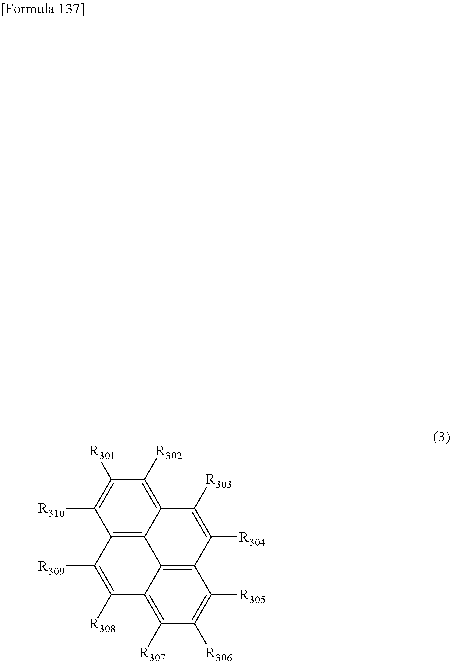

In the formula (3):

at least one combination of adjacent two or more of R₃₀₁ to R₃₁₀ are mutually bonded to form a substituted or unsubstituted monocyclic ring, mutually bonded to form a substituted or unsubstituted fused ring, or not mutually bonded;

at least one of R₃₀₁ to R₃₁₀ is a monovalent group represented by a formula (31) below; and

R₃₀₁ to R₃₁₀ forming neither the monocyclic ring nor the fused ring and not being the monovalent group represented by the formula (31) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

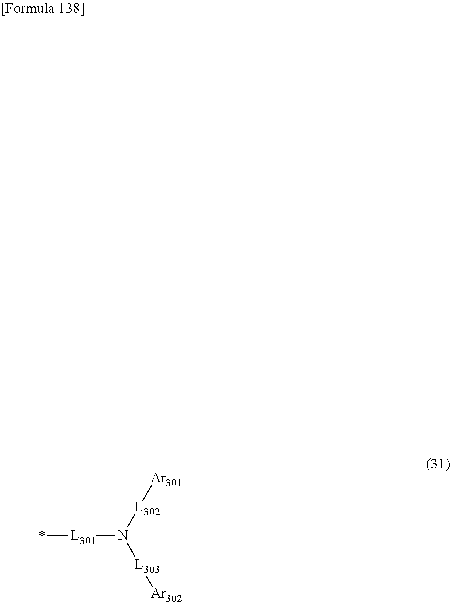

In the formula (31):

Ar₃₀₁ and Ar₃₀₂ are each independently a substituted or unsubstituted aryl group having 6 to 50 ring carbon atoms, or a substituted or unsubstituted heterocyclic group having 5 to 50 ring atoms;

L₃₀₁ to L₃₀₃ are each independently a single bond, a substituted or unsubstituted arylene group having 6 to 30 ring carbon atoms, or a substituted or unsubstituted divalent heterocyclic group having 5 to 30 ring atoms; and

* represents a bonding position to a pyrene ring in the formula (3).

In the third and fourth compounds, R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

a substituted or unsubstituted alkyl group having 1 to 50 carbon atoms, or a substituted or unsubstituted aryl group having 6 to 50 ring carbon atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different; and

when a plurality of R₉₀₇ are present, the plurality of R₉₀₇ are mutually the same or different.

In the formula (3), two of R₃₀₁ to R₃₁₀ are preferably groups represented by the formula (31).

In an exemplary embodiment, the compound represented by the formula (3) is a compound represented by a formula (33) below.

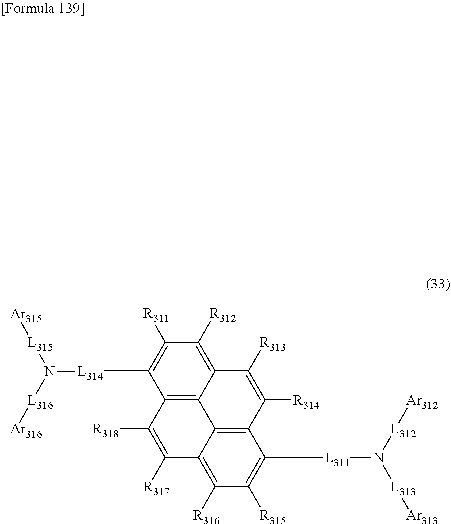

In the formula (33):

R₃₁₁ to R₃₁₈ each independently represent the same as R₃₀₁ to R₃₁₀ in the formula (3) that are not the monovalent group represented by the formula (31);

L₃₁₁ to L₃₁₆ are each independently a single bond, a substituted or unsubstituted arylene group having 6 to 30 ring carbon atoms, or a substituted or unsubstituted divalent heterocyclic group having 5 to 30 ring atoms; and

Ar₃₁₂, Ar₃₁₃, Ar₃₁₅, and Ar₃₁₆ are each independently a substituted or unsubstituted aryl group having 6 to 50 ring carbon atoms, or a substituted or unsubstituted heterocyclic group having 5 to 50 ring atoms.

In the formula (31), L₃₀₁ is preferably a single bond, and L₃₀₂ and L₃₀₃ are each preferably a single bond.

In an exemplary embodiment, the compound represented by the formula (3) is represented by a formula (34) or a formula (35) below.

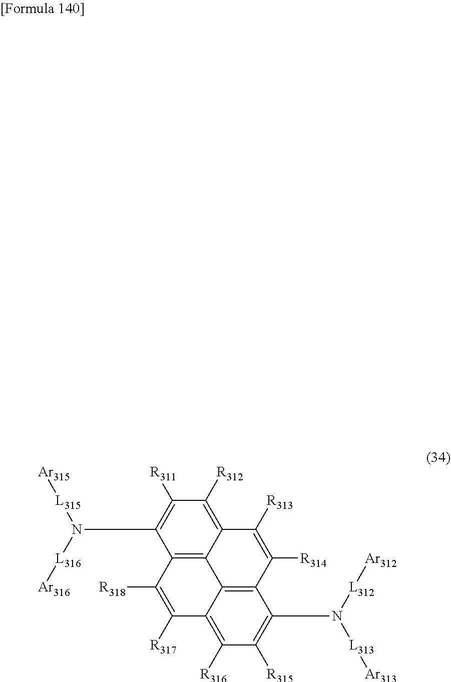

In the formula (34):

R₃₁₁ to R₃₁₈ each independently represent the same as R₃₀₁ to R₃₁₀ in the formula (3) that are not the monovalent group represented by the formula (31);

L₃₁₂, L₃₁₃, L₃₁₅ and L₃₁₆ each independently represent the same as L₃₁₂, L₃₁₃, L₃₁₅ and L₃₁₆ in the formula (33); and

Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

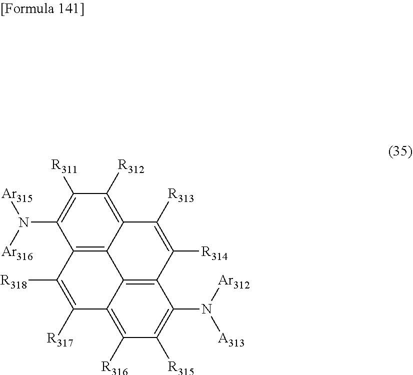

In the formula (35):

R₃₁₁ to R₃₁₈ each independently represent the same as R₃₀₁ to R₃₁₀ in the formula (3) that are not the monovalent group represented by the formula (31); and

Ar₃₁₂, Ar₃₁₃, Ar₃₁₅ and Ar₃₁₆ each independently represent the same as Ar₃₁₂, Ar₃₁₃, Ar₃₁₅ and Ar₃₁₆ in the formula (33).

In the formula (31), at least one of Ar₃₀₁ or Ar₃₀₂ is preferably a group represented by a formula (36) below.

In the formulae (33) to (35), at least one of Ar₃₁₂ or Ar₃₁₃ is preferably a group represented by the formula (36) below.

In the formulae (33) to (35), at least one of Ar₃₁₅ or Ar₃₁₆ is preferably a group represented by the formula (36) below.

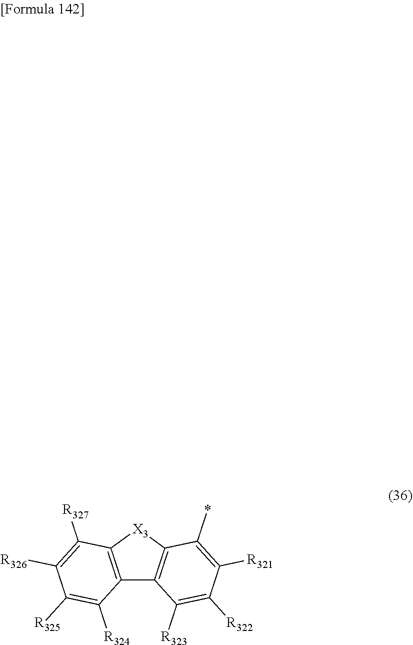

In the formula (36):

X₃ represents an oxygen atom or a sulfur atom;

at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;

R₃₂₁ to R₃₂₇ not forming the monocyclic ring and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and

* represents a bonding position to L₃₀₂, L₃₀₃, L₃₁₂, L₃₁₃, L₃₁₅ or L₃₁₆.

X₃ is preferably an oxygen atom.

At least one of R₃₂₁ to R₃₂₇ is preferably a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the formula (31), it is preferable that Ar₃₀₁ is the group represented by the formula (36) and Ar₃₀₂ is a substituted or unsubstituted aryl group having 6 to 50 ring carbon atoms.

In the formulae (33) to (35), it is preferable that Ar₃₁₂ is the group represented by the formula (36) and Ar₃₁₃ is a substituted or unsubstituted aryl group having 6 to 50 ring carbon atoms.

In the formulae (33) to (35), it is preferable that Ar₃₁₅ is the group represented by the formula (36) and Ar₃₁₆ is a substituted or unsubstituted aryl group having 6 to 50 ring carbon atoms.

In an exemplary embodiment, the compound represented by the formula (3) is represented by a formula (37) below.

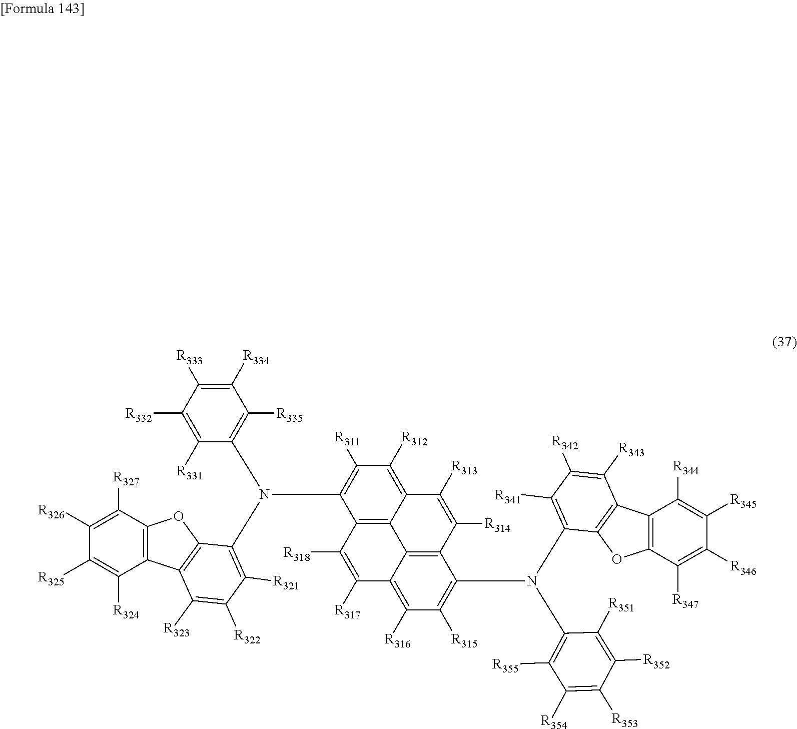

In the formula (37):

R₃₁₁ to R₃₁₈ each independently represent the same as R₃₀₁ to R₃₁₀ in the formula (3) that are not the monovalent group represented by the formula (31);

at least one combination of adjacent two or more of R₃₂₁ to R₃₂₇ are mutually bonded to form a substituted or unsubstituted monocyclic ring, mutually bonded to form a substituted or unsubstituted fused ring, or not mutually bonded;

at least one combination of adjacent two or more of R₃₄₁ to R₃₄₇ are mutually bonded to form a substituted or unsubstituted monocyclic ring, mutually bonded to form a substituted or unsubstituted fused ring, or not mutually bonded;

R₃₂₁ to R₃₂₇ and R₃₄₁ to R₃₄₇ neither forming the monocyclic ring nor forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and

R₃₃₁ to R₃₃₅ and R₃₅₁ to R₃₃₅ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of the compound represented by the formula (3) include compounds shown below.

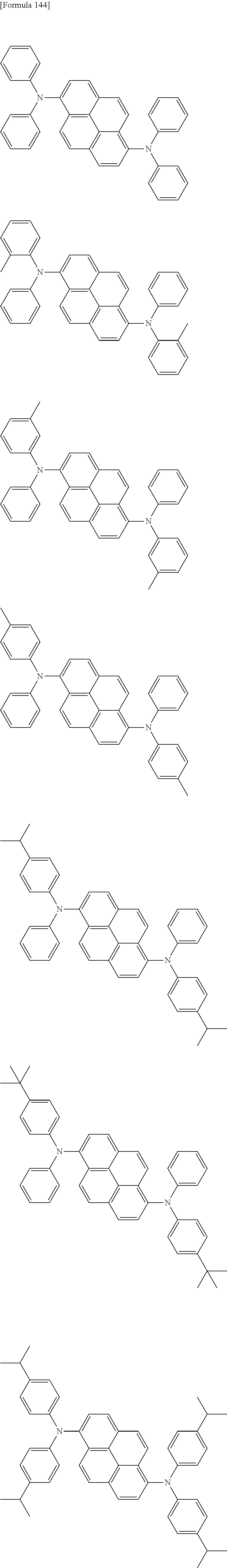

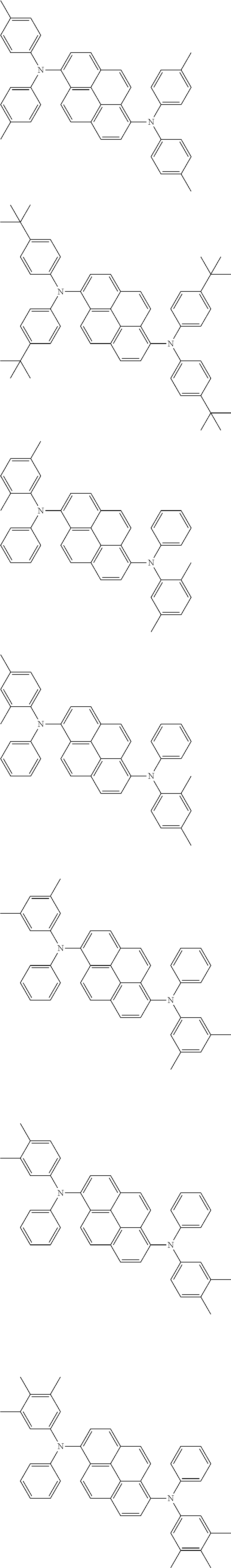

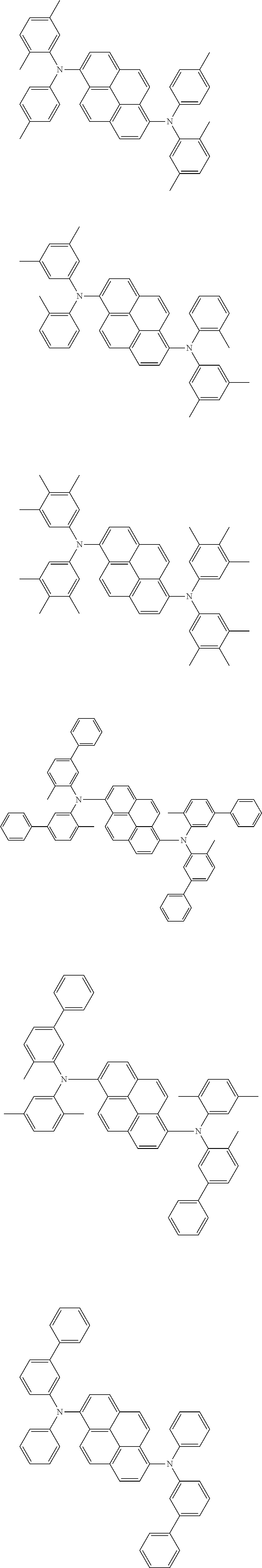

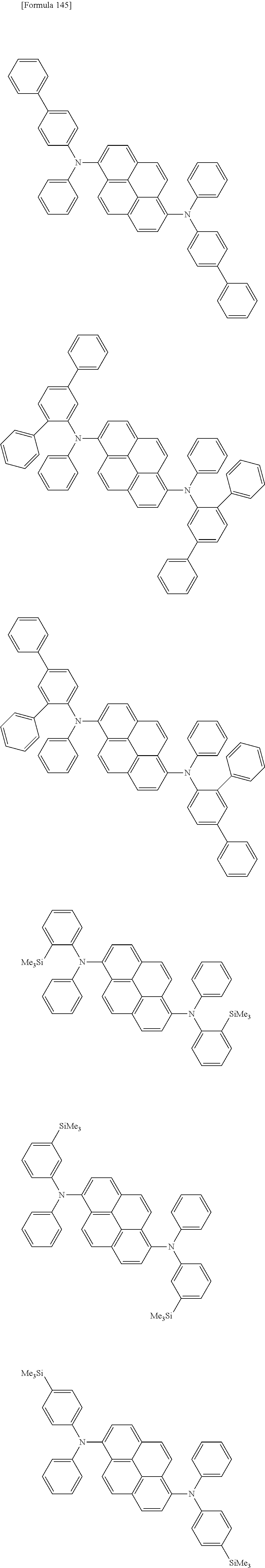

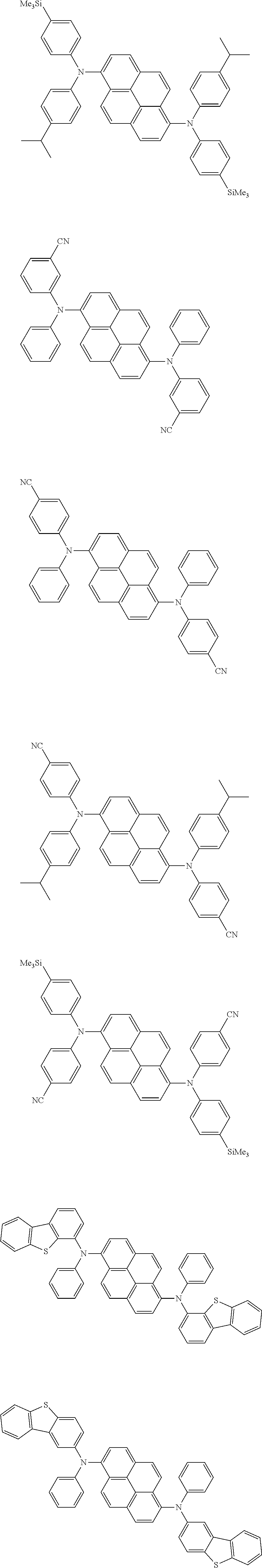

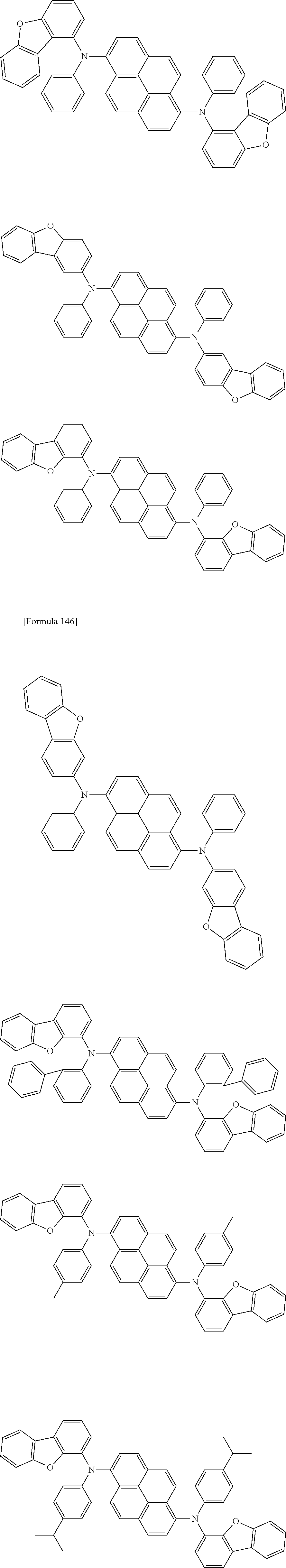

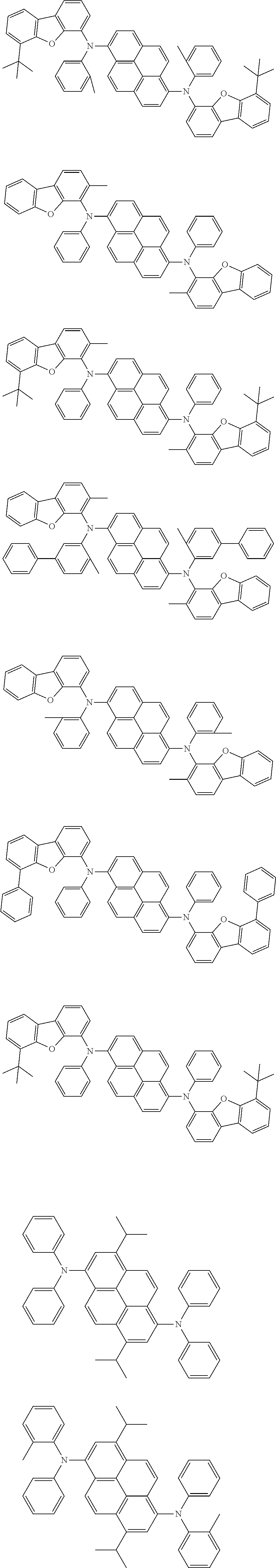

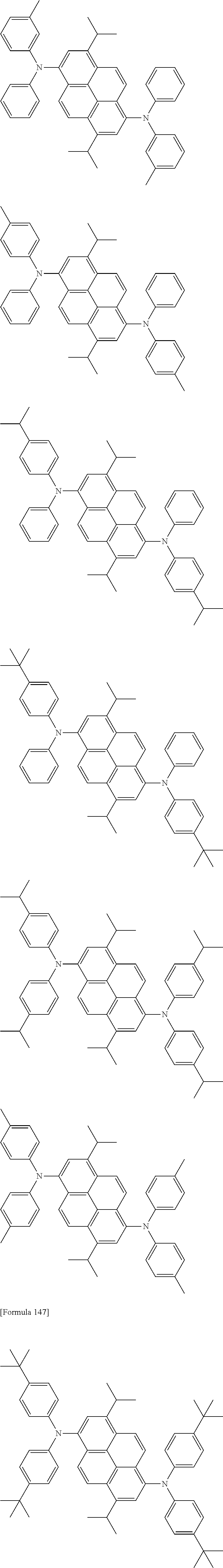

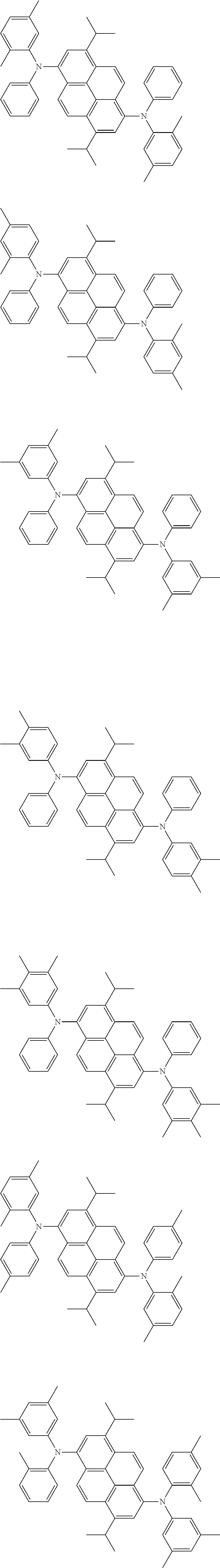

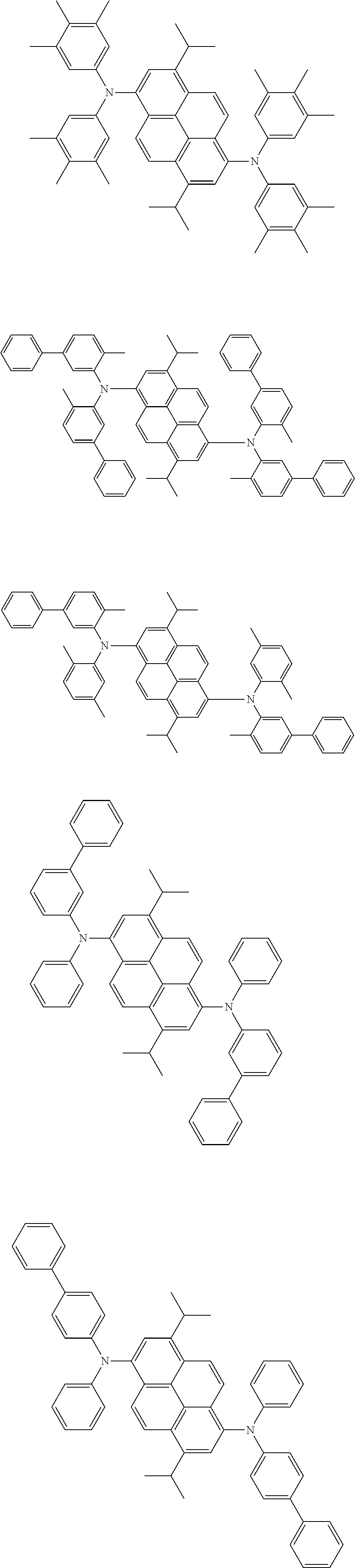

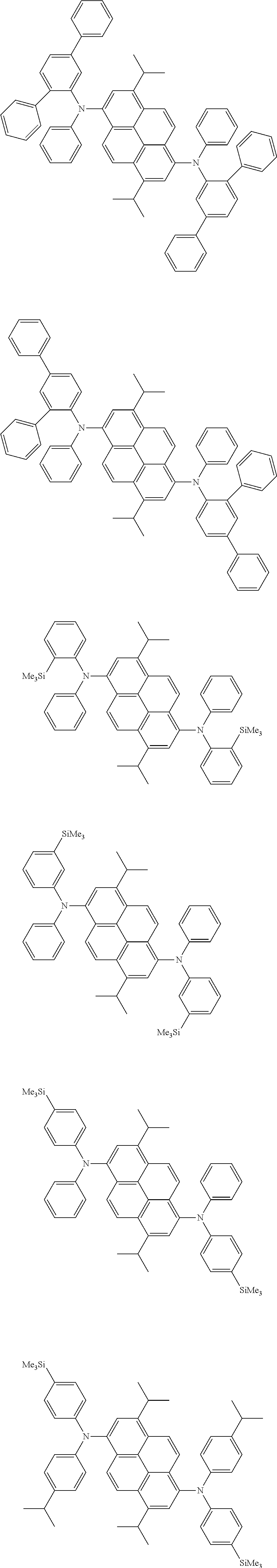

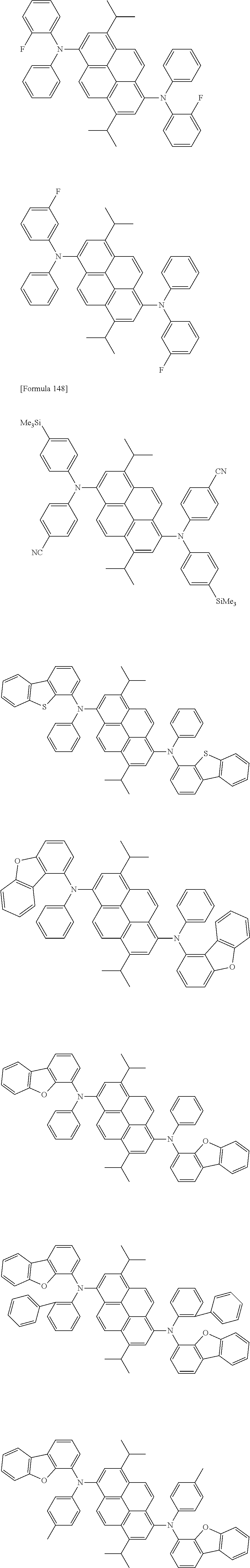

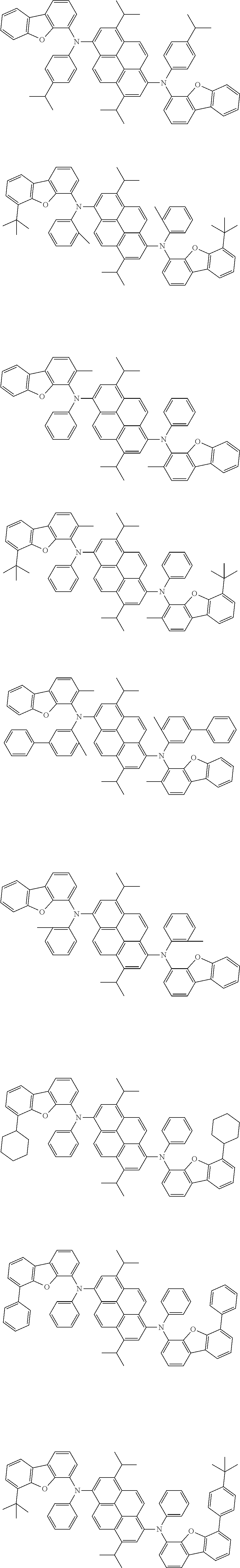

### Compound Represented by Formula (4)

The compound represented by the formula (4) will be described.

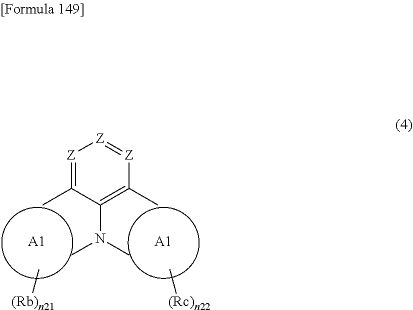

In the formula (4):

Z is each independently CRa or a nitrogen atom;

A1 ring and A2 ring are each independently a substituted or unsubstituted aromatic hydrocarbon ring having 6 to 50 ring carbon atoms, or a substituted or unsubstituted heterocycle having 5 to 50 ring atoms;

when a plurality of Ra are present, at least one combination of adjacent two or more of the plurality of Ra are mutually bonded to form a substituted or unsubstituted monocyclic ring, mutually bonded to form a substituted or unsubstituted fused ring, or not mutually bonded;

n21 and n22 are each independently 0, 1, 2, 3, or 4;

when a plurality of Rb are present, at least one combination of adjacent two or more of the plurality of Rb are mutually bonded to form a substituted or unsubstituted monocyclic ring, mutually bonded to form a substituted or unsubstituted fused ring, or not mutually bonded;

when a plurality of Rc are present, at least one combination of adjacent two or more of the plurality of Rc are mutually bonded to form a substituted or unsubstituted monocyclic ring, mutually bonded to form a substituted or unsubstituted fused ring, or not mutually bonded; and

Ra, Rb, and Rc not forming the monocyclic ring and not forming the fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

The “aromatic hydrocarbon ring” for the A1 ring and A2 ring has the same structure as the compound formed by introducing a hydrogen atom to the “aryl group” described above.

Ring atoms of the “aromatic hydrocarbon ring” for the A1 ring and the A2 ring include two carbon atoms on a fused bicyclic structure at the center of the formula (4).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the A1 ring and A2 ring has the same structure as the compound formed by introducing a hydrogen atom to the “heterocyclic group” described above.

Ring atoms of the “heterocycle” for the A1 ring and the A2 ring include two carbon atoms on a fused bicyclic structure at the center of the formula (4).

Specific examples of the “substituted or unsubstituted heterocycle having 5 to 50 ring atoms” include a compound formed by introducing a hydrogen atom to the “heterocyclic group” described in the specific example group G2.

Rb is bonded to any one of carbon atoms forming the aromatic hydrocarbon ring for the A1 ring or any one of the atoms forming the heterocycle for the A1 ring.

Rc is bonded to any one of carbon atoms forming the aromatic hydrocarbon ring for the A2 ring or any one of the atoms forming the heterocycle for the A2 ring.

At least one of Ra, Rb, or Rc is preferably a group represented by a formula (4a) below. More preferably, at least two of Ra, Rb, and Rc are groups represented by the formula (4a).

[Formula 150]

*-L₄₀₁-Ar₄₀₁ (4a)

In the formula (4a):

L₄₀₁ is a single bond, a substituted or unsubstituted arylene group having 6 to 30 ring carbon atoms, or a substituted or unsubstituted divalent heterocyclic group having 5 to 30 ring atoms; and

Ar₄₀₁ is a substituted or unsubstituted aryl group having 6 to 50 ring carbon atoms, a substituted or unsubstituted heterocyclic group having 5 to 50 ring atoms, or a group represented by a formula (4b) below.

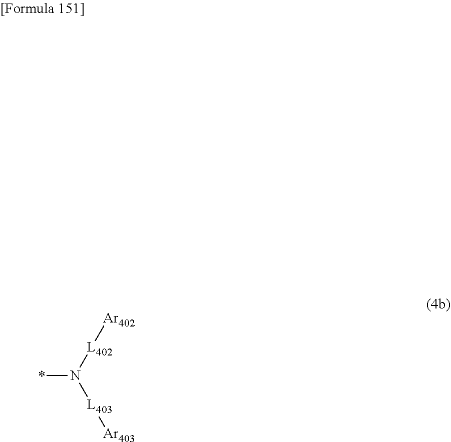

In the formula (4b):

L₄₀₂ and L₄₀₃ are each independently a single bond, a substituted or unsubstituted arylene group having 6 to 30 ring carbon atoms, or a substituted or unsubstituted divalent heterocyclic group having 5 to 30 ring atoms; a combination of Ar₄₀₂ and Ar₄₀₃ are mutually bonded to form a substituted or unsubstituted monocyclic ring, mutually bonded to form a substituted or unsubstituted fused ring, or not mutually bonded; and

Ar₄₀₂ and Ar₄₀₃ not forming the monocyclic ring and not forming the fused ring are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is represented by a formula (42) below.

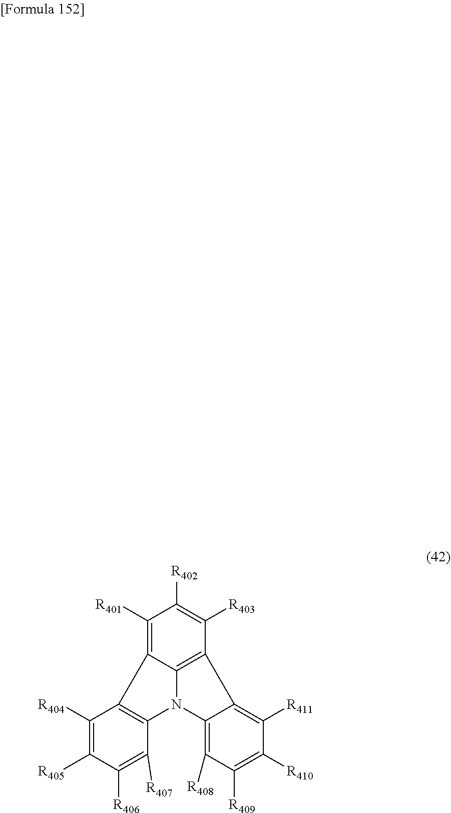

In the formula (42):

at least one combination of adjacent two or more of R₄₀₁ to R₄₁₁ are mutually bonded to form a substituted or unsubstituted monocyclic ring, mutually bonded to form a substituted or unsubstituted fused ring, or not mutually bonded; and

R₄₀₁ to R₄₁₁ neither forming the monocyclic ring nor forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one of R₄₀₁ to R₄₁₁ is preferably a group represented by the formula (4a). More preferably, at least two of R₄₀₁ to R₄₁₁ are groups represented by the formula (4a).

R₄₀₄ and R₄₁₁ are preferably groups represented by the formula (4a).

In an exemplary embodiment, the compound represented by the formula (4) is a compound formed by bonding a structure represented by a formula (4-1) or a formula (4-2) below to the A1 ring.

Further, in an exemplary embodiment, the compound represented by the formula (42) is a compound formed by bonding the structure represented by the formula (4-1) or the formula (4-2) to the ring bonded with R₄₀₄ to R₄₀₇.

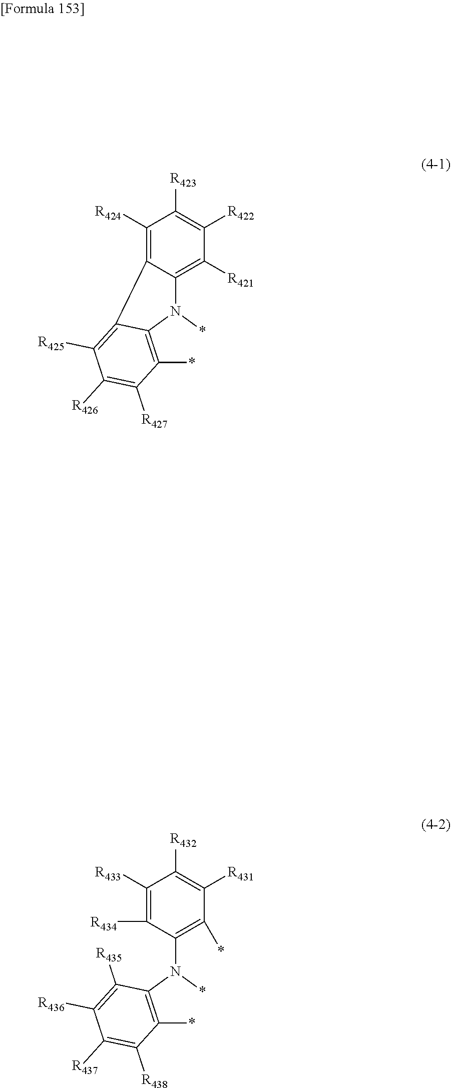

In the formula (4-1), two * are each independently bonded to the ring carbon atom of the aromatic hydrocarbon ring or the ring atom of the heterocycle as the A1 ring in the formula (4) or bonded to one of R₄₀₄ to R₄₀₇ in the formula (42).

In the formula (4-2), three * are each independently bonded to the ring carbon atom of the aromatic hydrocarbon ring or the ring atom of the heterocycle for the A1 ring in the formula (4) or bonded to one of R₄₀₄ to R₄₀₇ in the formula (42).

At least one combination of adjacent two or more of R₄₂₁ to R₄₂₇ are mutually bonded to form a substituted or unsubstituted monocyclic ring, mutually bonded to form a substituted or unsubstituted fused ring, or not mutually bonded.

At least one combination of adjacent two or more of R₄₃₁ to R₄₃₈ are mutually bonded to form a substituted or unsubstituted monocyclic ring, mutually bonded to form a substituted or unsubstituted fused ring, or not mutually bonded.

R₄₂₁ to R₄₂₇ and R₄₃₁ to R₄₃₈ neither forming the monocyclic ring nor forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is a compound represented by a formula (41-3), a formula (41-4), or a formula (41-5) below.

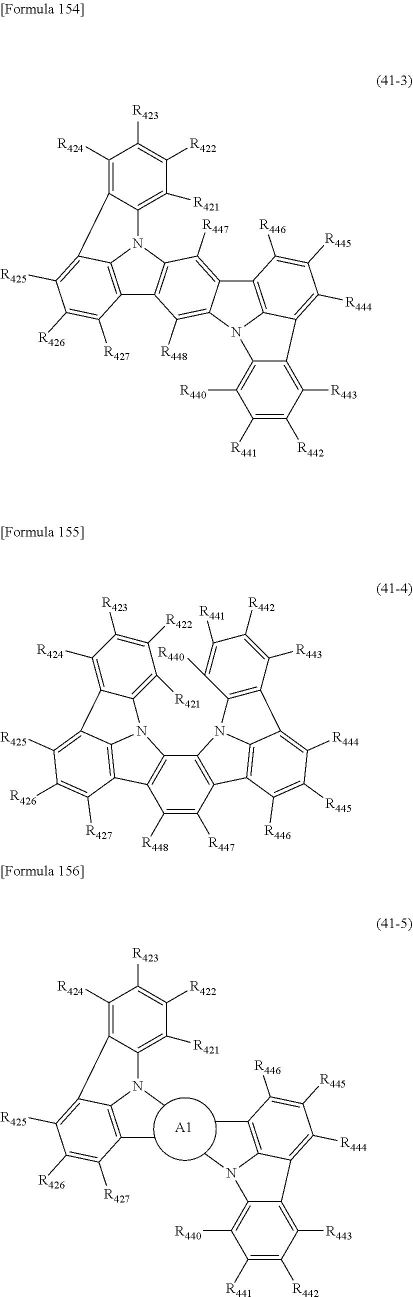

In the formulae (41-3), (41-4) and (41-5):

A1 ring is as defined for the formula (4);

R₄₂₁ to R₄₂₇ each independently represent the same as R₄₂₁ to R₄₂₇ in the formula (4-1); and

R₄₄₀ to R₄₄₈ each independently represent the same as R₄₀₁ to R₄₁₁ in the formula (42).

In an exemplary embodiment, a substituted or unsubstituted aromatic hydrocarbon ring having 6 to 50 ring carbon atoms for the A1 ring in the formula (41-5) is a substituted or unsubstituted naphthalene ring, or a substituted or unsubstituted fluorene ring.

In an exemplary embodiment, a substituted or unsubstituted heterocycle having 5 to 50 ring atoms for the A1 ring in the formula (41-5) is a substituted or unsubstituted dibenzofuran ring, a substituted or unsubstituted carbazole ring, or a substituted or unsubstituted dibenzothiophene ring.

In an exemplary embodiment, the compound represented by the formula (4) or the formula (42) is a compound selected from the group consisting of compounds represented by formulae (461) to (467) below.

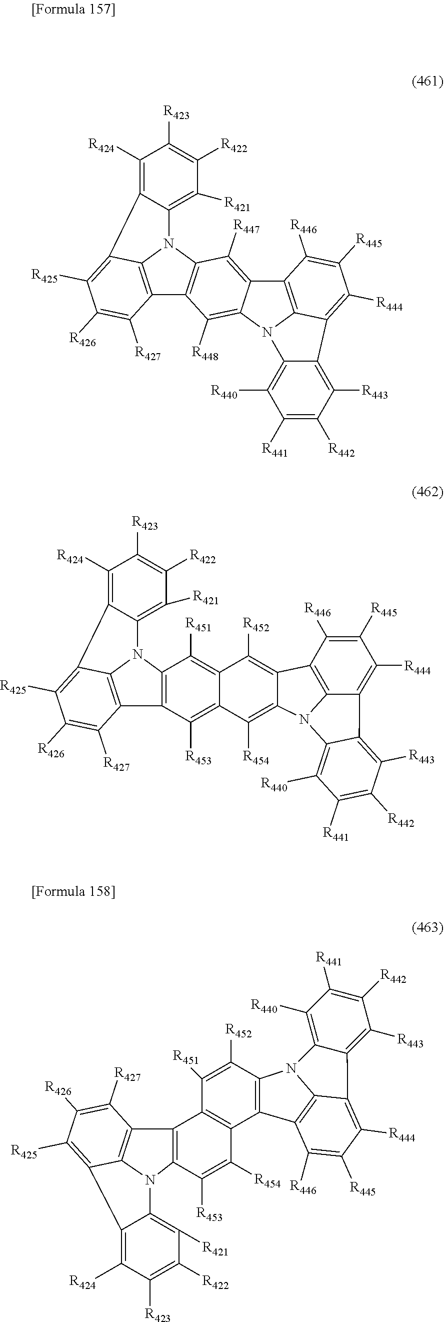

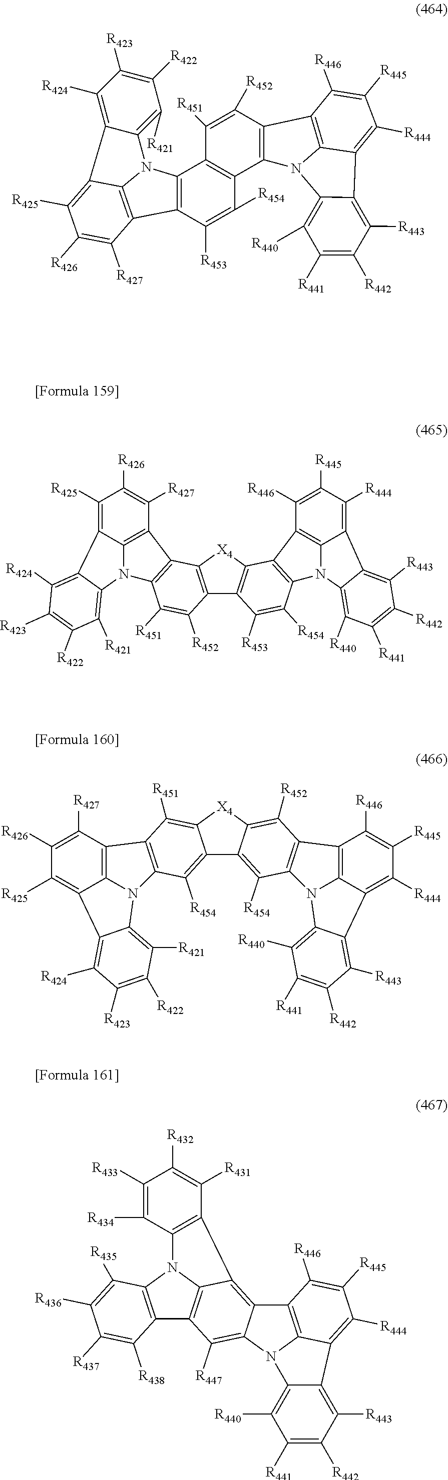

In the formulae (461), (462), (463), (464), (465), (466) and (467):

R₄₂₁ to R₄₂₇ each independently represent the same as R₄₂₁ to R₄₂₇ in the formula (4-1);

R₄₃₁ to R₄₃₈ each independently represent the same as R₄₃₁ to R₄₃ in the formula (4-2);

R₄₄₀ to R₄₄₈ and R₄₅₁ to R₄₅₄ each independently represent the same as R₄₀₁ to R₄₁₁ in the formula (42);

X₄ is an oxygen atom, NR₈₀₁, or C(R₈₀₂)(R₈₀₃);

R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

a substituted or unsubstituted alkyl group having 1 to 50 carbon atoms, or a substituted or unsubstituted aryl group having 6 to 50 ring carbon atoms;

when a plurality of R₈₀₁ are present, the plurality of R₈₀₁ are mutually the same or different;

when a plurality of R₈₀₂ are present, the plurality of R₈₀₂ are mutually the same or different; and

when a plurality of R₈₀₃ are present, the plurality of R₈₀₃ are mutually the same or different.

In an exemplary embodiment, in the compound represented by the formula (42), at least one combination of adjacent two or more of R₄₀₁ to R₄₁₁ are mutually bonded to form a substituted or unsubstituted monocyclic ring or a substituted or unsubstituted fused ring. The compound represented by the formula (42) in the exemplary embodiment is described in detail as a compound represented by a formula (45).

### Compound Represented by Formula (45)

The compound represented by the formula (45) will be described.

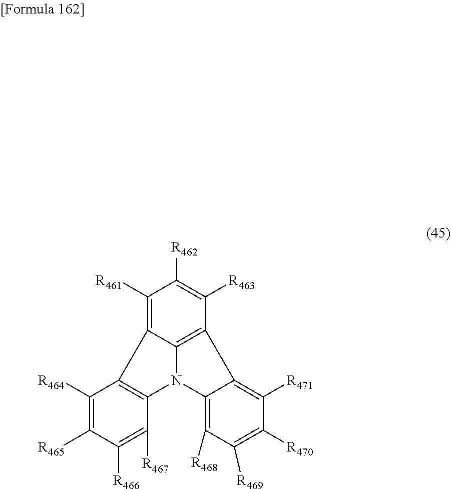

In the formula (45):

two or more of combinations selected from the group consisting of a combination of R₄₆₁ and R₄₆₂, a combination of R₄₆₂ and R₄₆₃, a combination of R₄₆₄ and R₄₆₅, a combination of R₄₆₅ and R₄₆₆, a combination of R₄₆₆ and R₄₆₇, a combination of R₄₆₈ and R₄₆₉, a combination of R₄₆₉ and R₄₇₀, and a combination of R₄₇₀ and R₄₇₁ are mutually bonded to form a substituted or unsubstituted monocyclic ring or a substituted or unsubstituted fused ring.

However, the combination of R₄₆₁ and R₄₆₂ and the combination of R₄₆₂ and R₄₆₃, the combination of R₄₆₄ and R₄₆₅ and the combination of R₄₆₅ and R₄₆₆, the combination of R₄₆₅ and R₄₆₆ and the combination of R₄₆₆ and R₄₆₇, the combination of R₄₆₈ and R₄₆₉ and the combination of R₄₆₉ and R₄₇₀, and the combination of R₄₆₉ and R₄₇₀ and the combination of R₄₇₀ and R₄₇₁ do not form a ring at the same time.

At least two rings formed by R₄₆₁ to R₄₇₁ are mutually the same or different.

R₄₆₁ to R₄₇₁ neither forming the monocyclic ring nor forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (45), Rₙ and Rₙ₊₁ (n being an integer selected from 461, 462, 464 to 466, and 468 to 470) are mutually bonded to form a substituted or unsubstituted monocyclic ring or fused ring together with two ring carbon atoms bonded with Rₙ and Rₙ₊₁. The ring is preferably formed of atoms selected from the group consisting of a carbon atom, an oxygen atom, a sulfur atom, and a nitrogen atom, and is preferably made of 3 to 7, more preferably 5 or 6 atoms.

The number of the above cyclic structures in the compound represented by the formula (45) is, for instance, 2, 3, or 4. The two or more of the cyclic structures may be present on the same benzene ring on the basic skeleton represented by the formula (45) or may be present on different benzene rings. For instance, when three cyclic structures are present, each of the cyclic structures may be present on corresponding one of the three benzene rings of the formula (45).

Examples of the above cyclic structures in the compound represented by the formula (45) include structures represented by formulae (451) to (460) below.

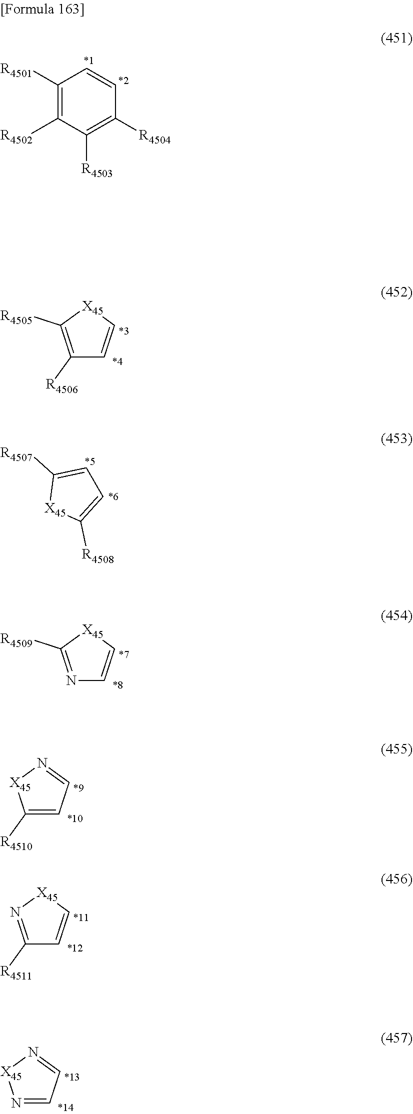

In the formulae (451) to (457):

each combination of *1 and *2, *3 and *4, *5 and *6, *7 and *8, *9 and *10, *11 and *12, and *13 and *14 represent the two ring carbon atoms bonded with Rₙ and Rₙ₊₁;

the ring carbon atom bonded with Rₙ may be any one of the two ring carbon atoms represented by *1 and *2, *3 and *4, *5 and *6, *7 and *8, *9 and *10, *11 and *12, and *13 and *14.

X₄₅ is C(R₄₅₁₂)(R₄₅₁₃), NR₄₅₁₄, an oxygen atom, or a sulfur atom;

at least one combination of adjacent two or more of R₄₅₀₁ to R₄₅₀₆ and R₄₅₁₂ to R₄₅₁₃ are mutually bonded to form a substituted or unsubstituted monocyclic ring, mutually bonded to form a substituted or unsubstituted fused ring, or not mutually bonded; and

R₄₅₀₁ to R₄₅₁₄ not forming the monocyclic ring and not forming the fused ring each independently represent the same as R₄₆₁ to R₄₇₁ in the formula (45).

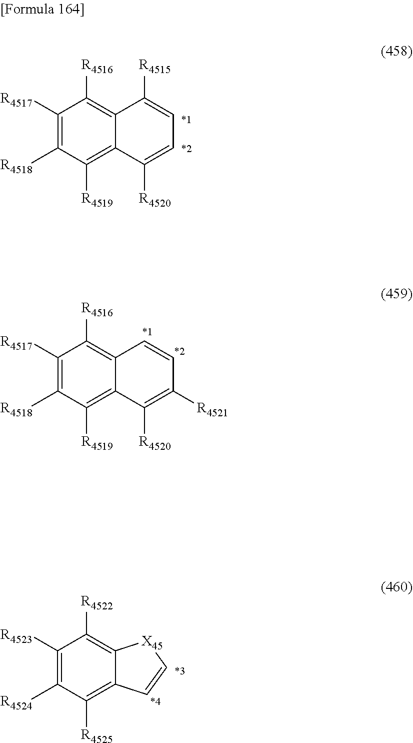

In the formulae (458) to (460):

each combination of *1 and *2, and *3 and *4 represent the two ring carbon atoms bonded with Rₙ and Rₙ₊₁;

the ring carbon atom bonded with Rₙ may be any one of the two ring carbon atoms represented by *1 and *2, or *3 and *4;

X₄₅ is C(R₄₅₁₂)(R₄₅₁₃), NR₄₅₁₄, an oxygen atom, or a sulfur atom;

at least one combination of adjacent two or more of R₄₅₁₂ to R₄₅₁₃ and R₄₅₁₅ to R₄₅₂₅ are mutually bonded to form a substituted or unsubstituted monocyclic ring, mutually bonded to form a substituted or unsubstituted fused ring, or not mutually bonded; and

R₄₅₁₂ and R₄₅₁₃, R₄₅₁₅ to R₄₅₂₁, and R₄₅₂₂ to R₄₅₂₅ not forming the monocyclic ring and not forming the fused ring, and R₄₅₁₄ each independently represent the same as R₄₆₁ to R₄₇₁ in the formula (45).

In the formula (45), it is preferable that at least one of R₄₆₂, R₄₆₄, R₄₆₅, R₄₇₀ or R₄₇₁ (preferably, at least one of R₄₆₂, R₄₆₅ or R₄₇₀, more preferably R₄₆₂) is a group not forming the cyclic structure.

(i) A substituent, if present, of the cyclic structure formed by Rₙ and Rₙ₊₁ of the formula (45),

(ii) R₄₆₁ to R₄₇₁ not forming the cyclic structure in the formula (45), and

(iii) R₄₅₀₁ to R₄₅₁₄, R₄₅₁₅ to R₄₅₂₅ in the formulae (451) to (460) are preferably each independently any one of groups selected from the group consisting of a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), a substituted or unsubstituted aryl group having 6 to 50 ring carbon atoms, a substituted or unsubstituted heterocyclic group having 5 to 50 ring atoms, or groups represented by formulae (461) to (464).

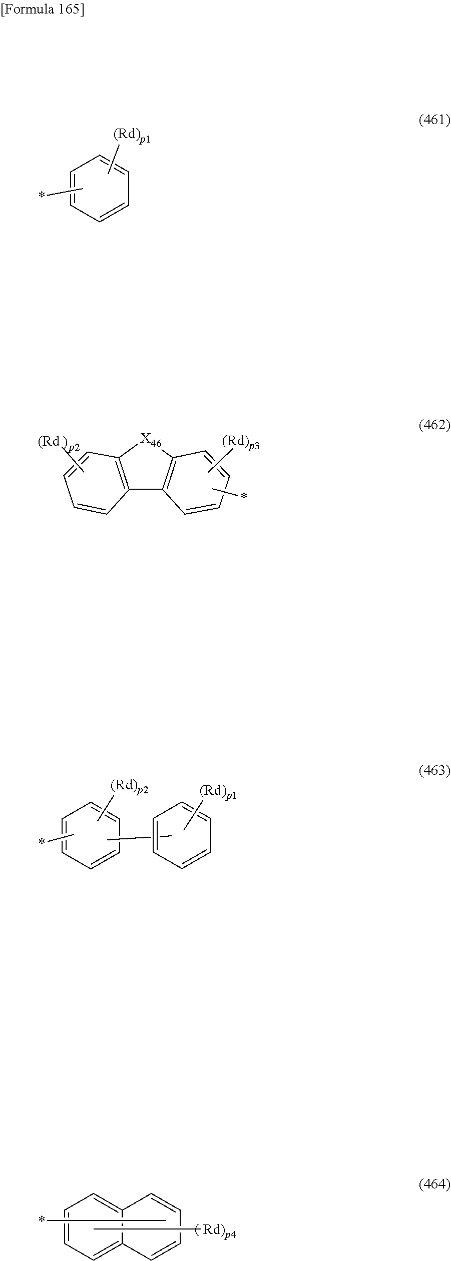

In the formulae (461) to (464):

R_{d} are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms,

X₄₆ is C(R₈₀₁)(R₈₀₂), NR₈₀₃, an oxygen atom or a sulfur atom;

R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

a substituted or unsubstituted alkyl group having 1 to 50 carbon atoms, or a substituted or unsubstituted aryl group having 6 to 50 ring carbon atoms;

when a plurality of R₈₀₁ are present, the plurality of R₈₀₁ are mutually the same or different;

when a plurality of R₈₀₂ are present, the plurality of R₈₀₂ are mutually the same or different;

when a plurality of R₈₀₃ are present, the plurality of R₈₀₃ are mutually the same or different;

p1 is 5;

p2 is 4;

p3 is 3;

p4 is 7; and

* in the formulae (461) to (464) each independently represent a bonding position to a cyclic structure.

In the third and fourth compounds, R₉₀₁ to R₉₀₇ represent the same as those as described above.

In an exemplary embodiment, the compound represented by the formula (45) is represented by one of formulae (45-1) to (45-6) below.

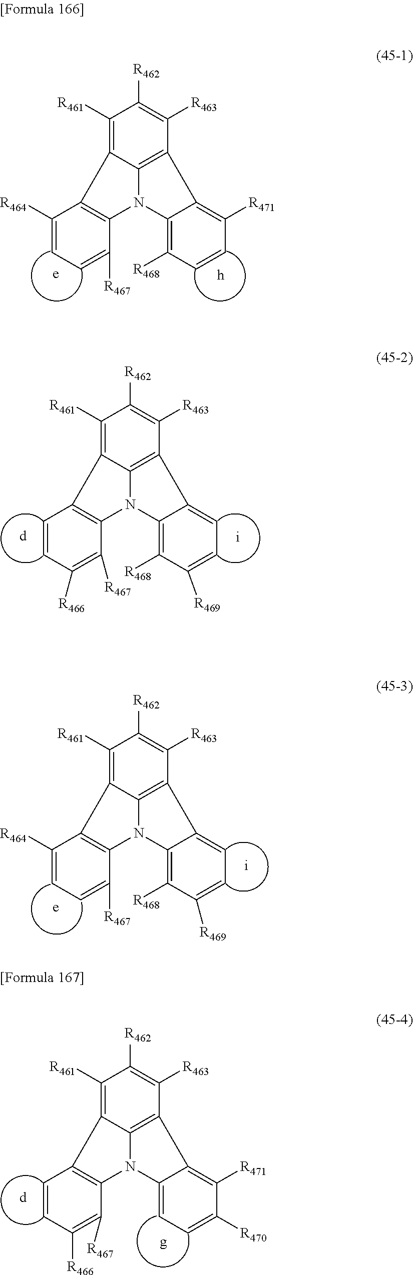

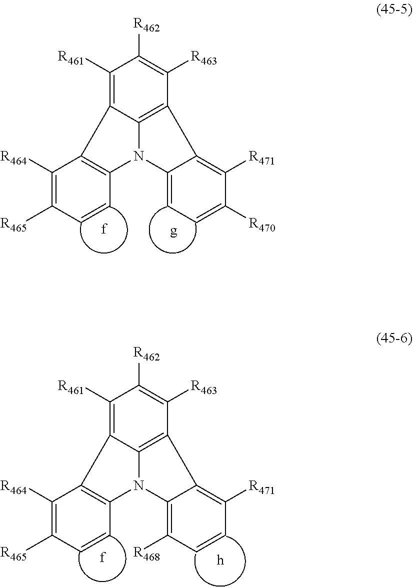

In the formulae (45-1) to (45-6):

rings d to i are each independently a substituted or unsubstituted monocyclic ring or a substituted or unsubstituted fused ring; and

R₄₆₁ to R₄₇₁ each independently represent the same as R₄₆₁ to R₄₇₁ in the formula (45).

In an exemplary embodiment, the compound represented by the formula (45) is represented by one of formulae (45-7) to (45-12) below.

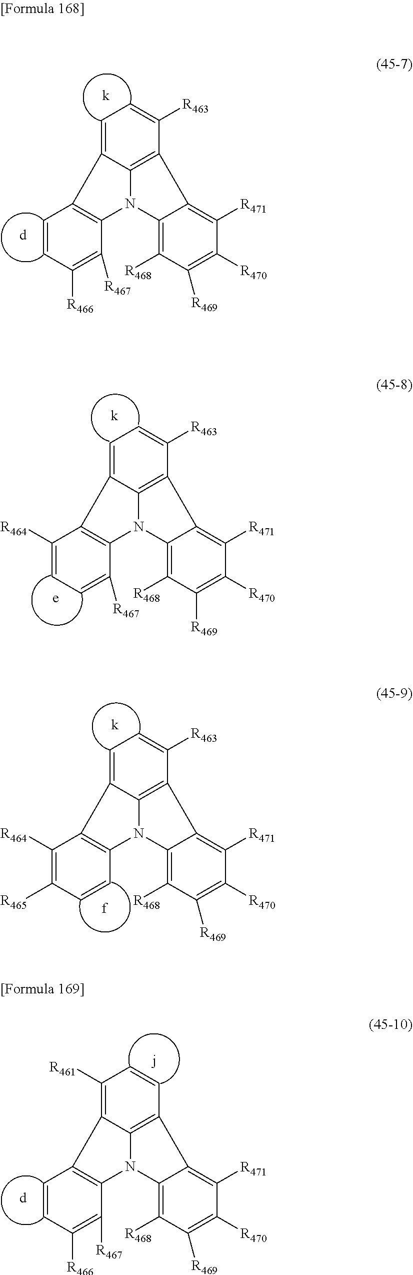

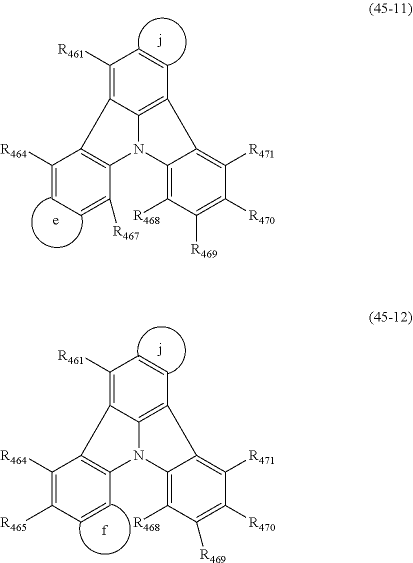

In the formulae (45-7) to (45-12):

rings d to f, k and j are each independently a substituted or unsubstituted monocyclic ring or a substituted or unsubstituted fused ring; and

R₄₆₁ to R₄₇₁ each independently represent the same as R₄₆₁ to R₄₇₁ in the formula (45).

In an exemplary embodiment, the compound represented by the formula (45) is represented by one of formulae (45-13) to (45-21) below.

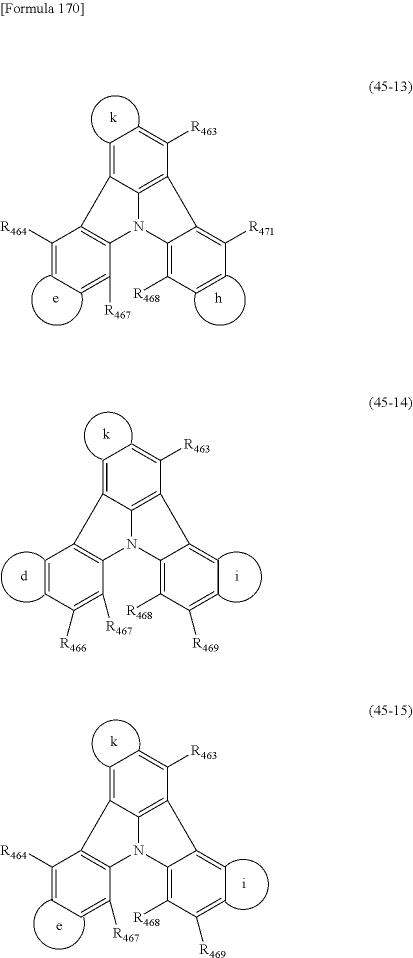

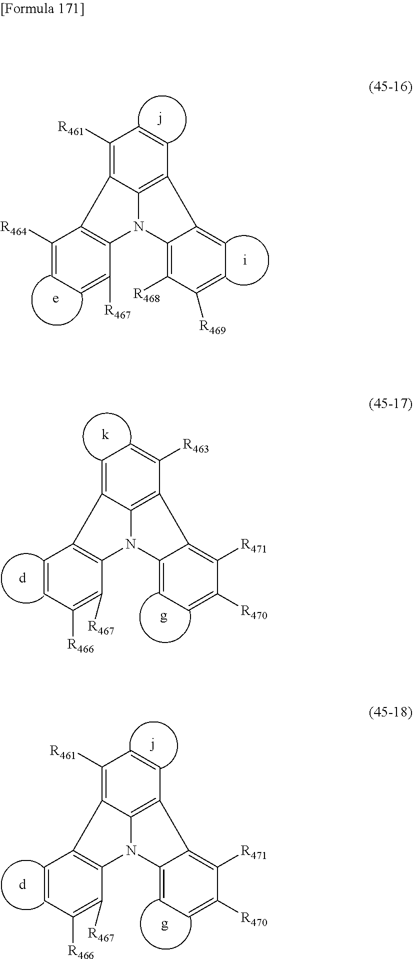

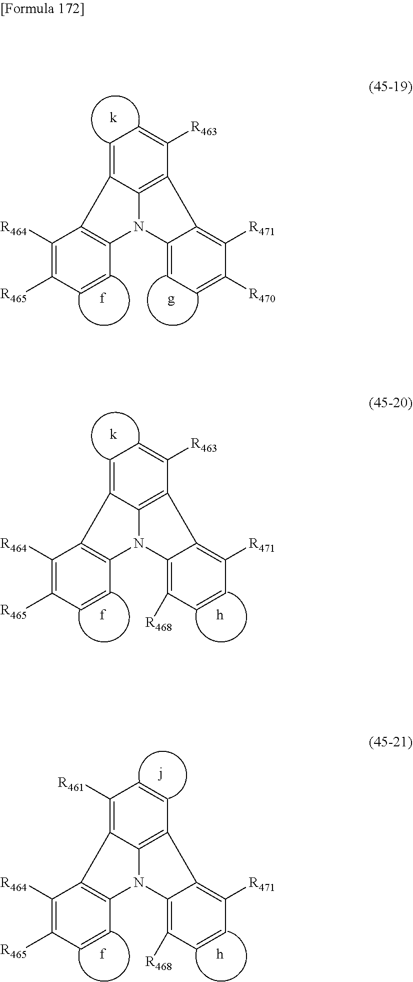

In the formulae (45-13) to (45-21):

rings d to k are each independently a substituted or unsubstituted monocyclic ring or a substituted or unsubstituted fused ring; and

R₄₆₁ to R₄₇₁ each independently represent the same as R₄₆₁ to R₄₇₁ in the formula (45).

When the ring g or the ring h further has a substituent, examples of the substituent include a substituted or unsubstituted alkyl group having 1 to 50 carbon atoms, a substituted or unsubstituted aryl group having 6 to 50 ring carbon atoms, a group represented by the formula (461), a group represented by the formula (463), and a group represented by the formula (464).

In an exemplary embodiment, the compound represented by the formula (45) is represented by one of formulae (45-22) to (45-25) below.

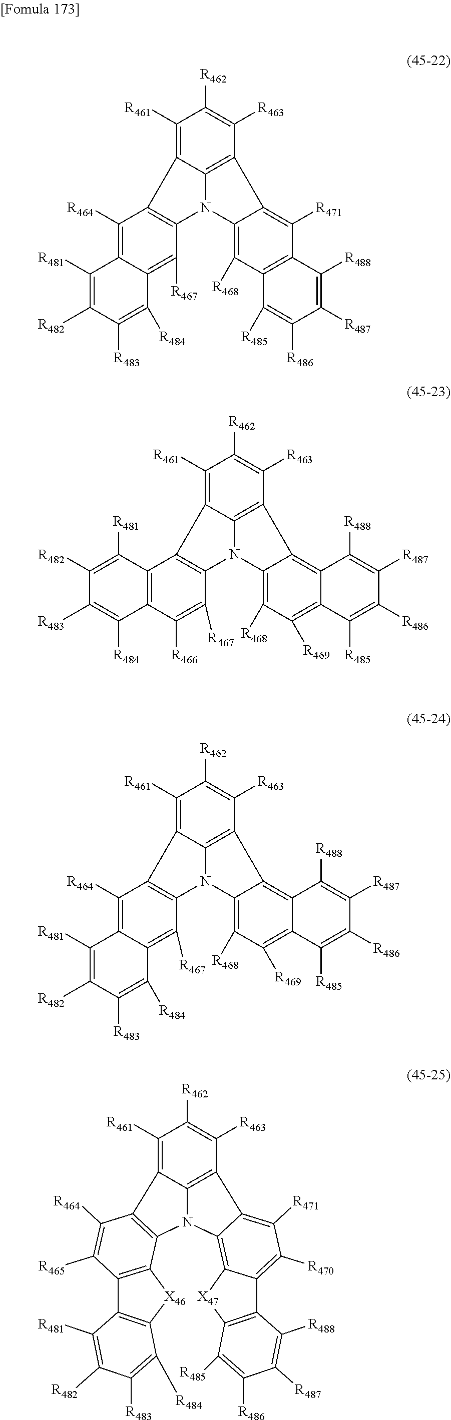

In the formulae (45-22) to (45-25):

X₄₆ and X₄₇ are each independently C(R₈₀₁)(R₈₀₂), NR₈₀₃, an oxygen atom or a sulfur atom; and

R₄₆₁ to R₄₇₁ and R₄₈₁ to R₄₈₈ each independently represent the same as R₄₆₁ to R₄₇₁ of the formula (45).

R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

a substituted or unsubstituted alkyl group having 1 to 50 carbon atoms, or a substituted or unsubstituted aryl group having 6 to 50 ring carbon atoms;

when a plurality of R₈₀₁ are present, the plurality of R₈₀₁ are mutually the same or different;

when a plurality of R₈₀₂ are present, the plurality of R₈₀₂ are mutually the same or different; and

when a plurality of R₈₀₃ are present, the plurality of R₈₀₃ are mutually the same or different.

In an exemplary embodiment, the compound represented by the formula (45) is represented by a formula (45-26) below.

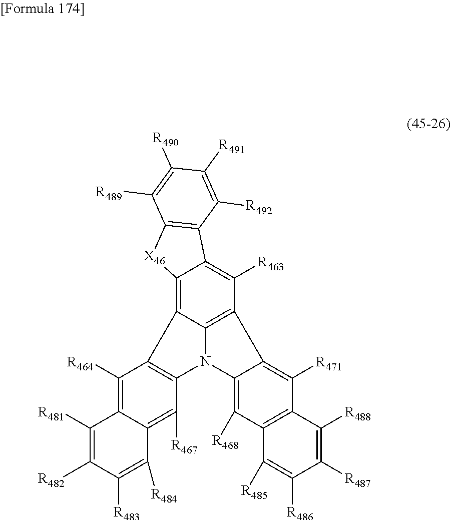

In the formula (45-26):

X₄₆ is C(R₈₀₁)(R₈₀₂), NR₈₀₃, an oxygen atom or a sulfur atom;

R₄₆₃, R₄₆₄, R₄₆₇, R₄₆₈, R₄₇₁, and R₄₈₁ to R₄₉₂ each independently represent the same as R₄₆₁ to R₄₇₁ in the formula (45);

R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

a substituted or unsubstituted alkyl group having 1 to 50 carbon atoms, or a substituted or unsubstituted aryl group having 6 to 50 ring carbon atoms;

when a plurality of R₈₀₁ are present, the plurality of R₈₀₁ are mutually the same or different;

when a plurality of R₈₀₂ are present, the plurality of R₈₀₂ are mutually the same or different; and

when a plurality of R₈₀₃ are present, the plurality of R₈₀₃ are mutually the same or different.

Specific examples of the compound represented by the formula (4) include compounds shown below. In the specific examples below, Ph represents a phenyl group, and D represents a deuterium atom.

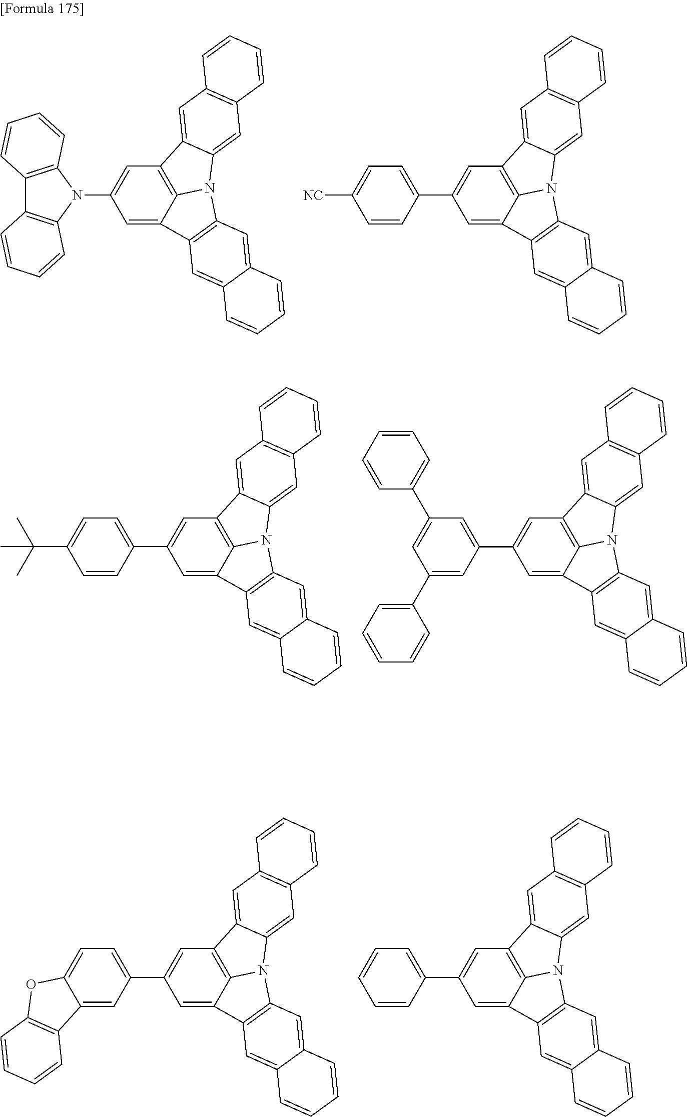

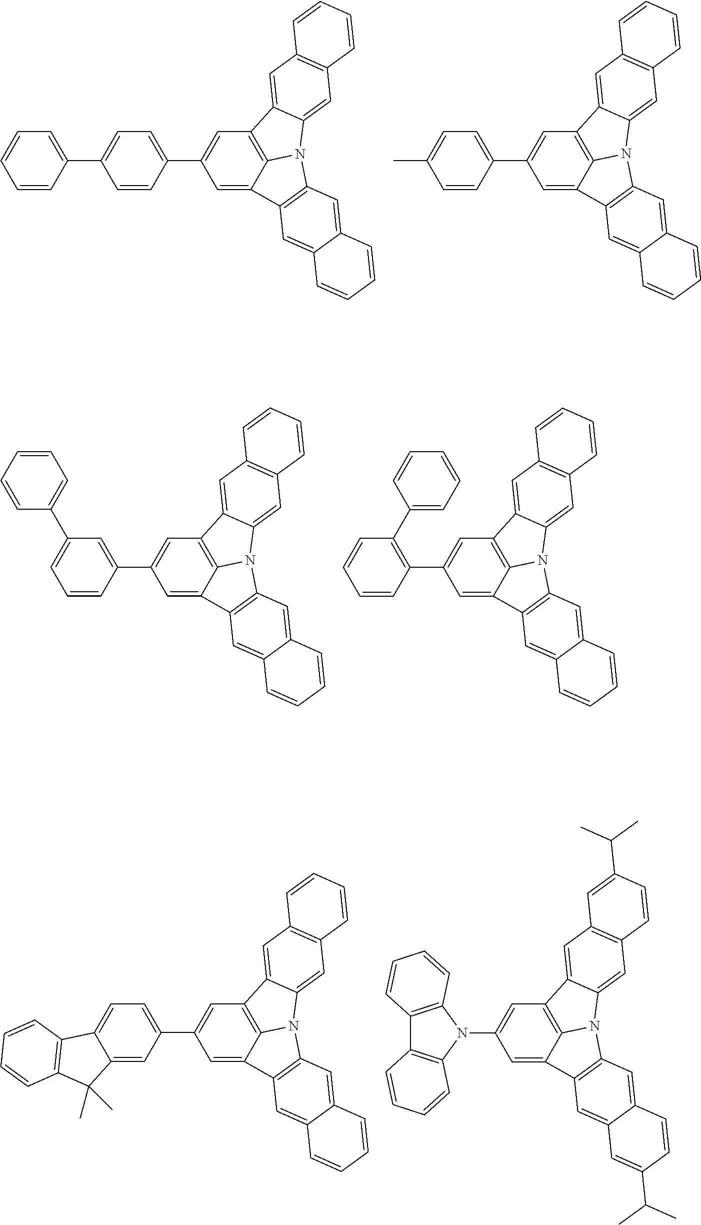

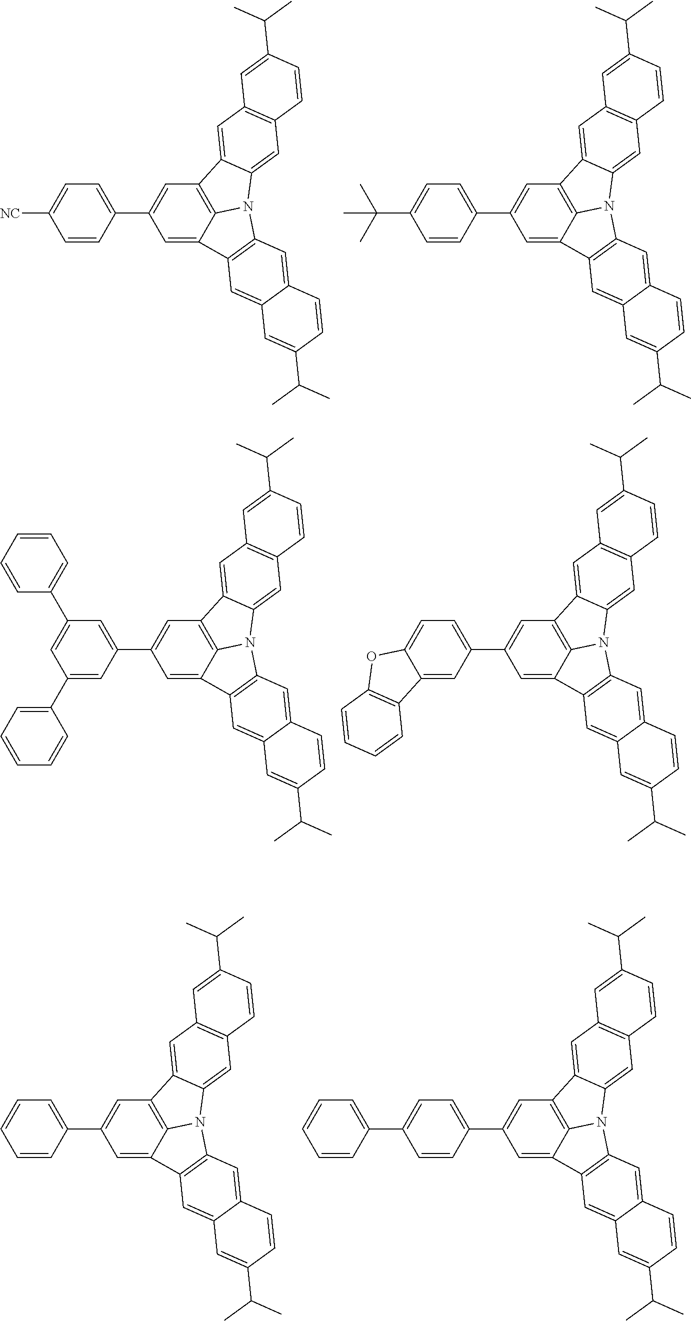

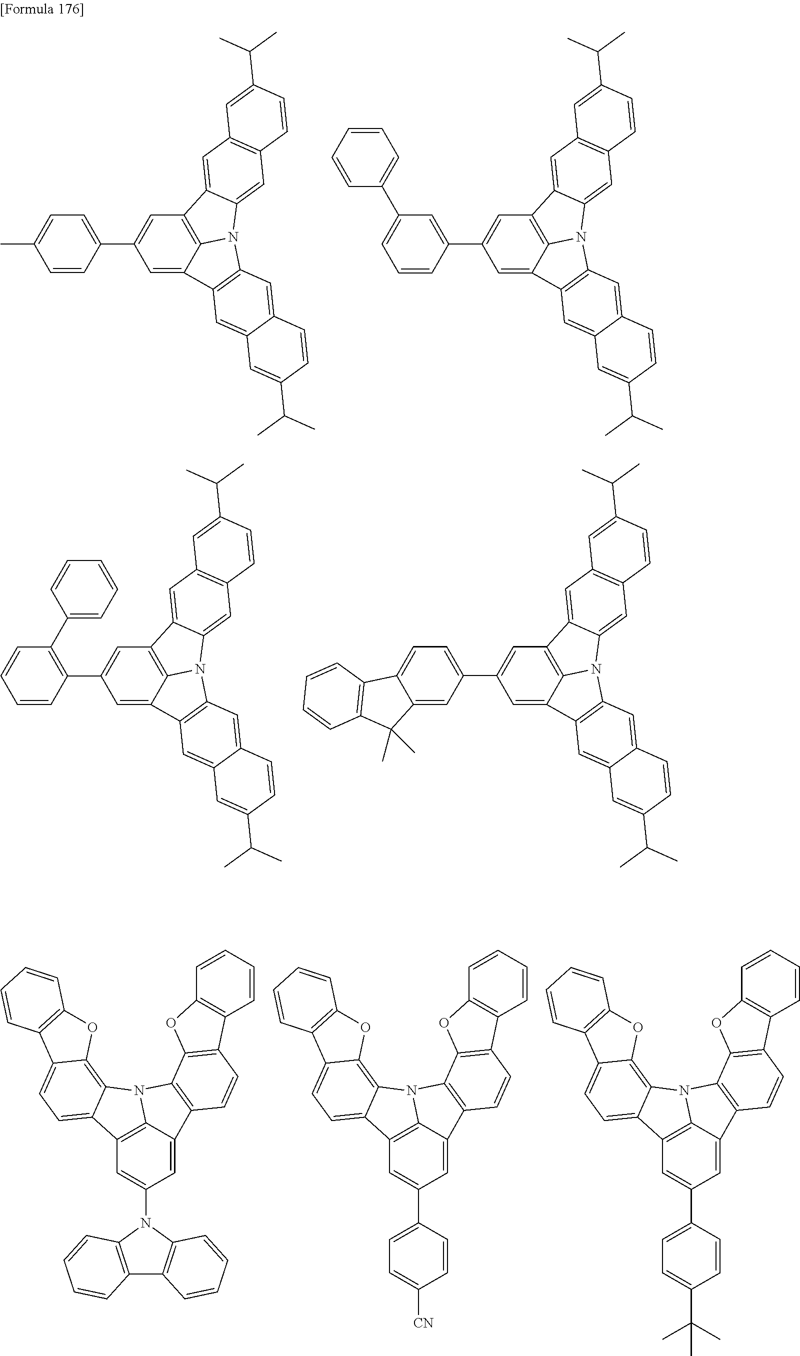

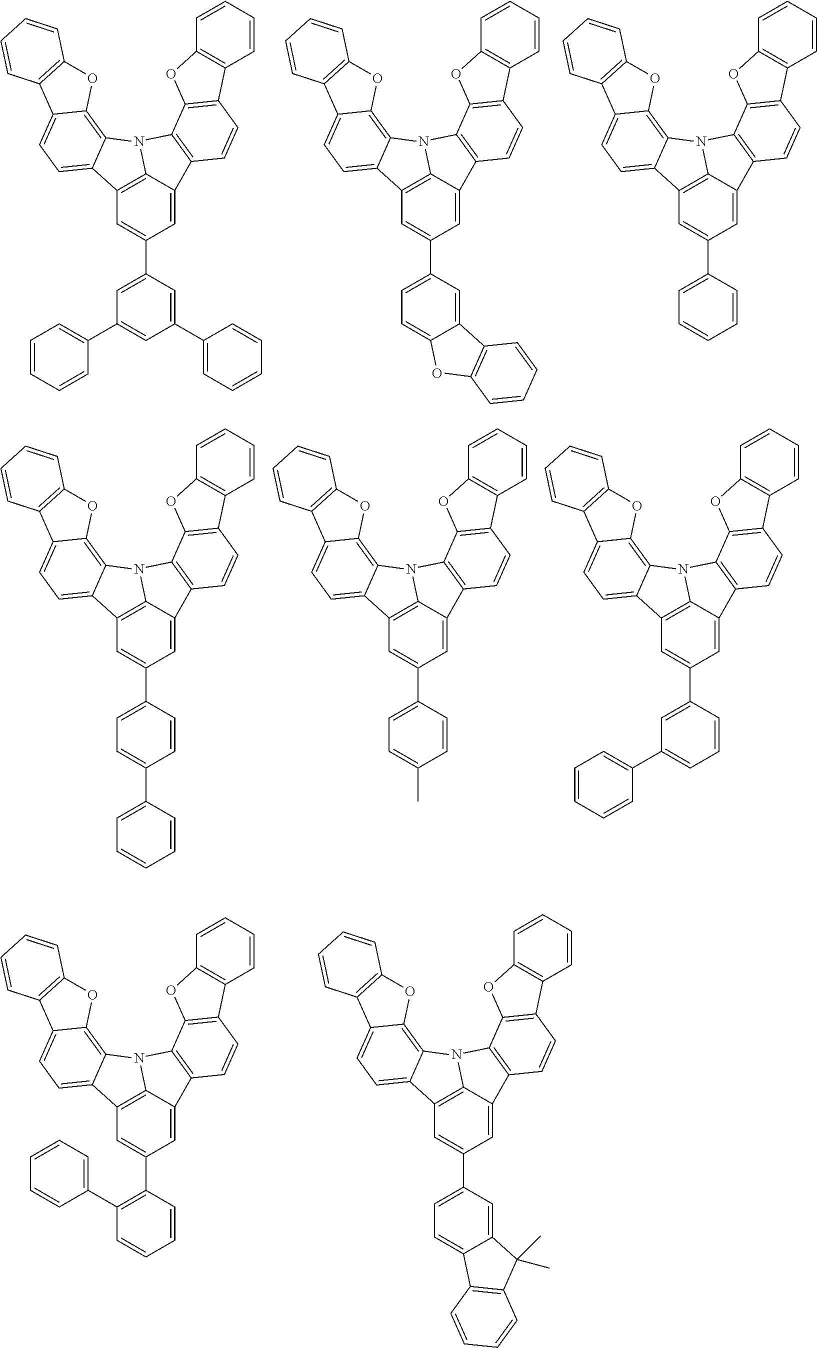

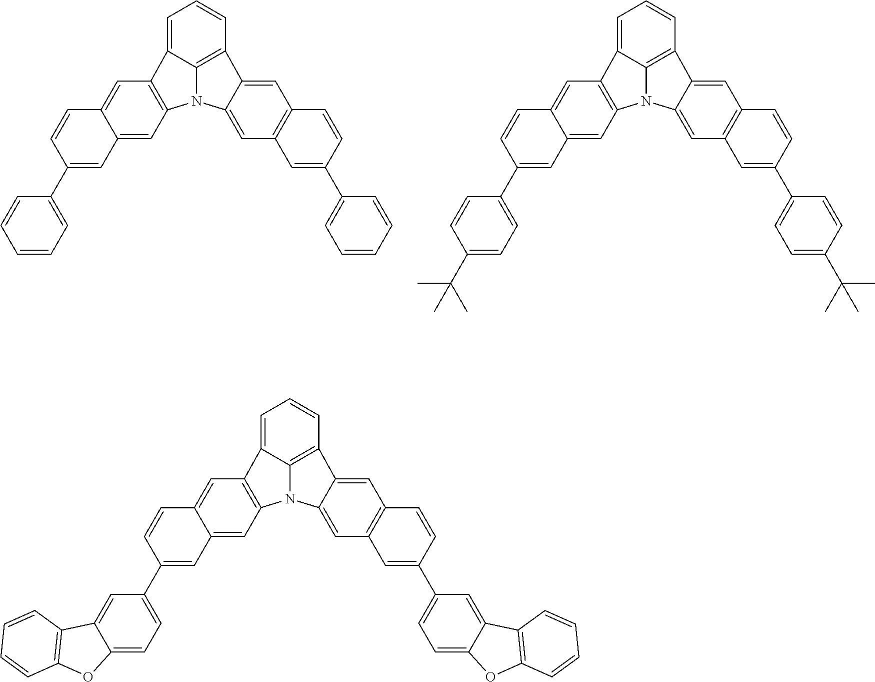 [SMILES: c1ccc(-c2ccc3cc4c5cccc6c7cc8ccc(-c9ccccc9)cc8cc7n(c4cc3c2)c56)cc1 ; CC(C)(C)c1ccc(-c2ccc3cc4c5cccc6c7cc8ccc(-c9ccc(C(C)(C)C)cc9)cc8cc7n(c4cc3c2)c56)cc1 ; c1ccc2c(c1)oc1ccc(-c3ccc4cc5c6cccc7c8cc9ccc(-c%10ccc%11oc%12ccccc%12c%11c%10)cc9cc8n(c5cc4c3)c67)cc12]

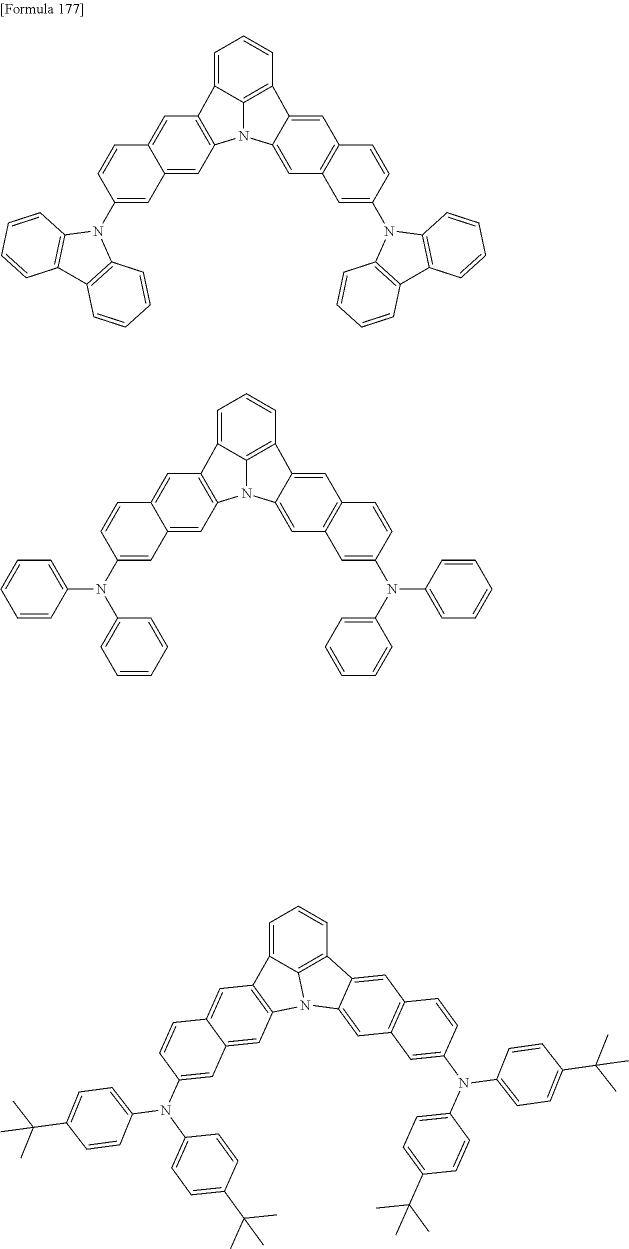 [SMILES: c1ccc2c(c1)c1ccccc1n2-c1ccc2cc3c4cccc5c6cc7ccc(-n8c9ccccc9c9ccccc98)cc7cc6n(c3cc2c1)c45 ; c1ccc(N(c2ccccc2)c2ccc3cc4c5cccc6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cccc6c7cc8ccc(N(c9ccc(C(C)(C)C)cc9)c9ccc(C(C)(C)C)cc9)cc8cc7n(c4cc3c2)c56)cc1]

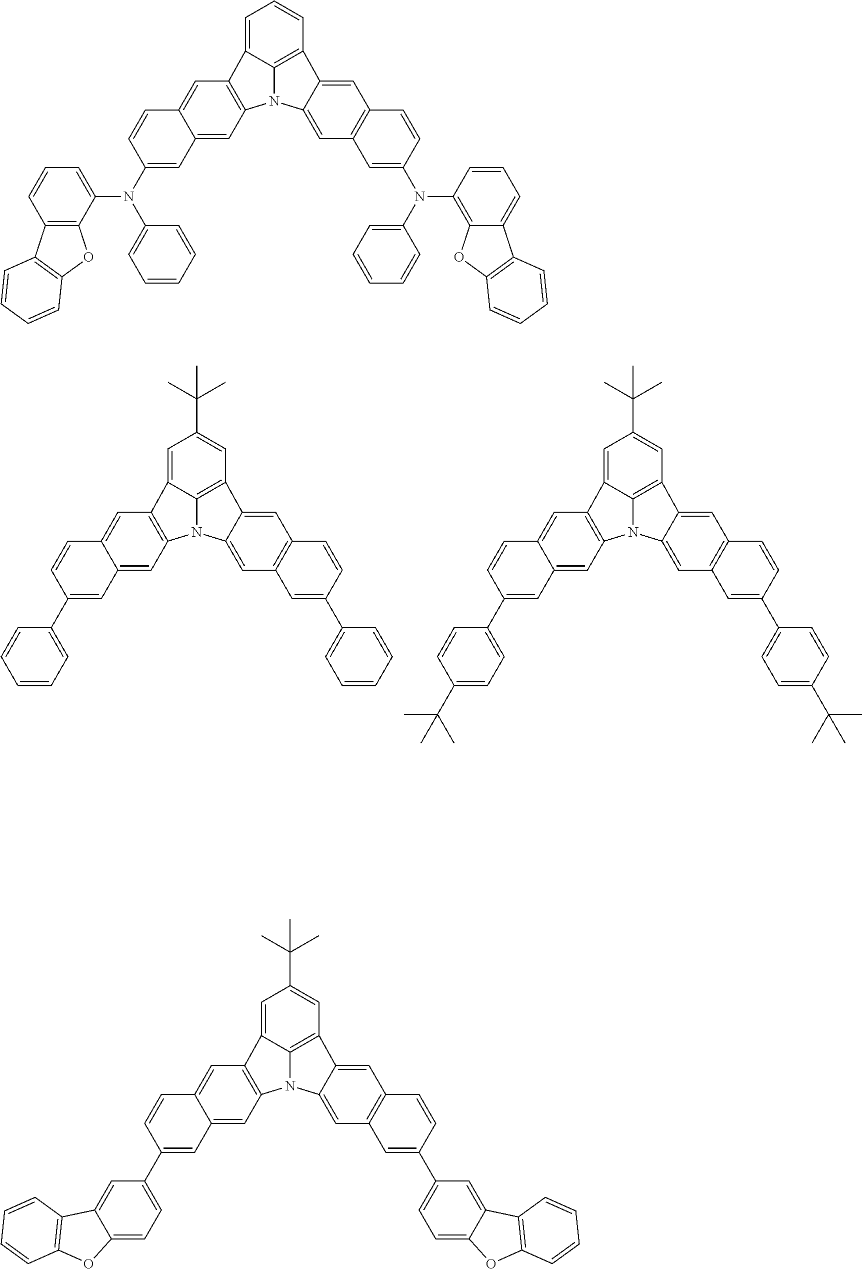

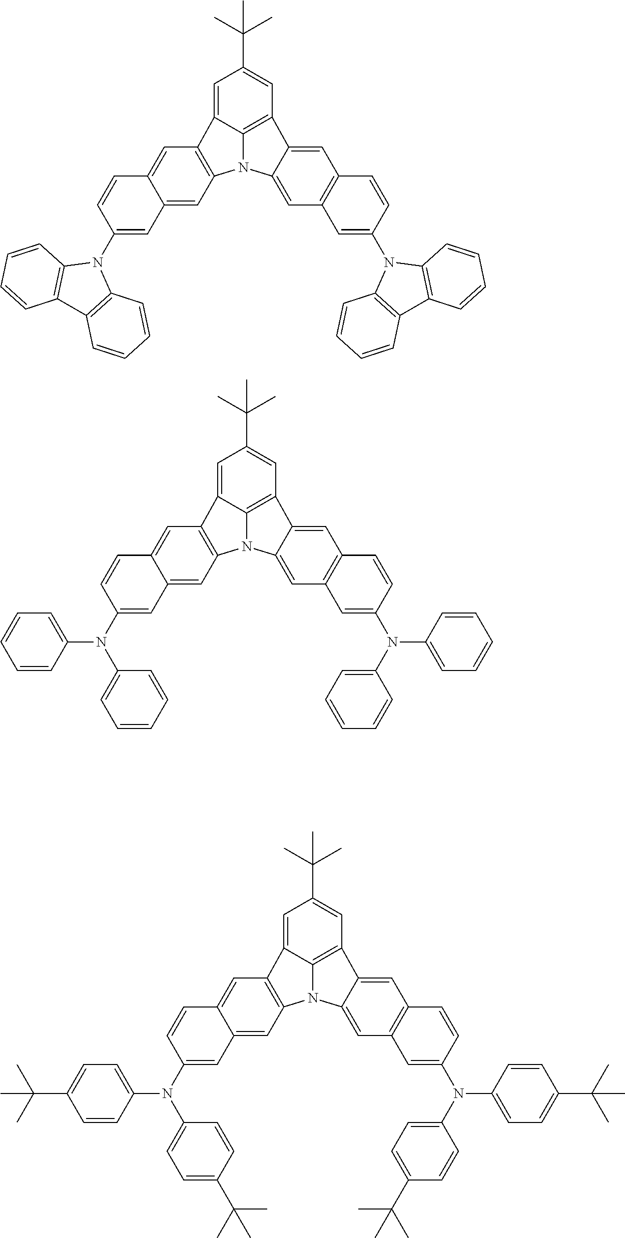 [SMILES: CC(C)(C)c1cc2c3cc4ccc(-n5c6ccccc6c6ccccc65)cc4cc3n3c4cc5cc(-n6c7ccccc7c7ccccc76)ccc5cc4c(c1)c23 ; CC(C)(C)c1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C(C)(C)C)cc6c7cc8ccc(N(c9ccc(C(C)(C)C)cc9)c9ccc(C(C)(C)C)cc9)cc8cc7n(c4cc3c2)c56)cc1]

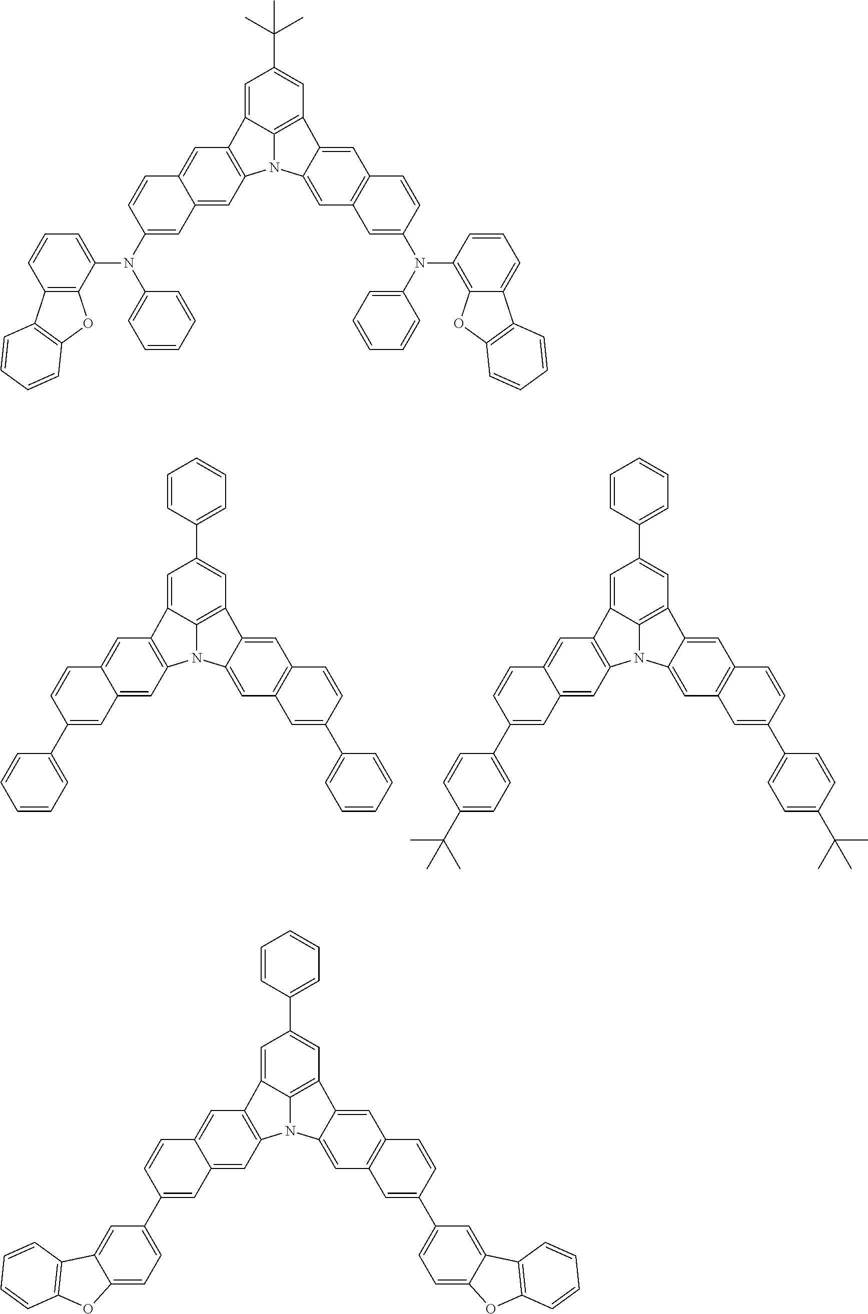

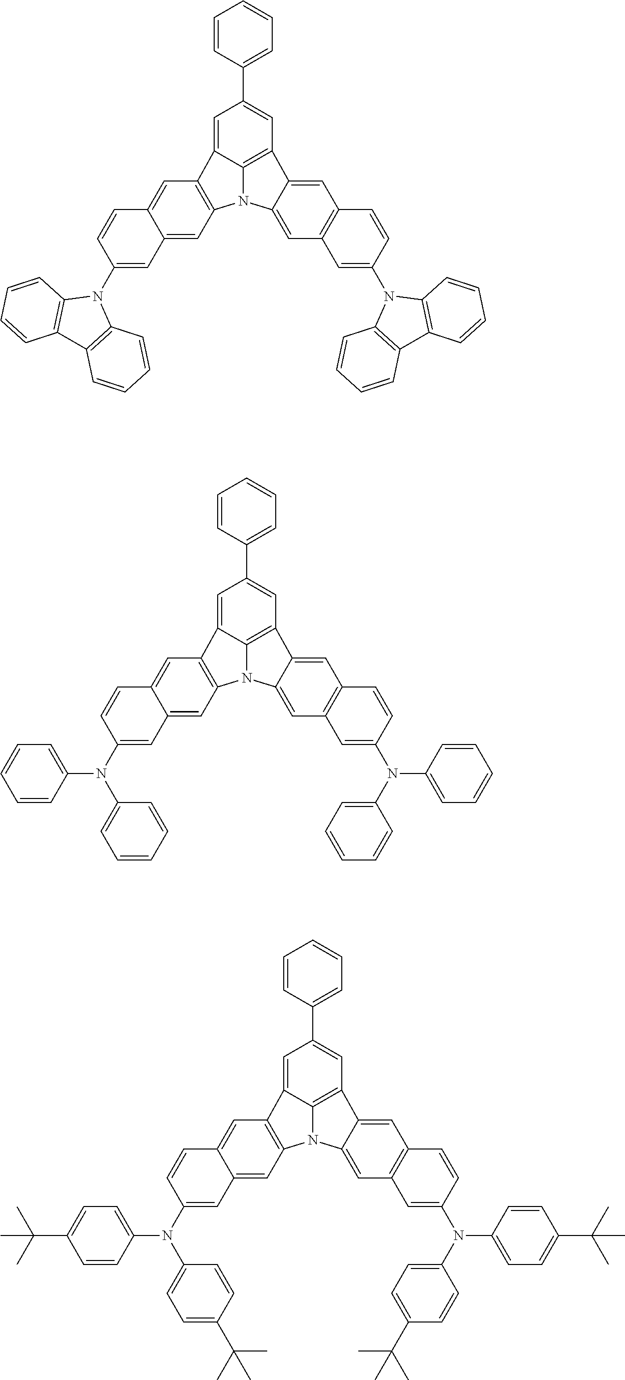 [SMILES: c1ccc(-c2cc3c4cc5ccc(-n6c7ccccc7c7ccccc76)cc5cc4n4c5cc6cc(-n7c8ccccc8c8ccccc87)ccc6cc5c(c2)c34)cc1 ; c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; CC(C)(C)c1ccc(N(c2ccc(C(C)(C)C)cc2)c2ccc3cc4c5cc(-c6ccccc6)cc6c7cc8ccc(N(c9ccc(C(C)(C)C)cc9)c9ccc(C(C)(C)C)cc9)cc8cc7n(c4cc3c2)c56)cc1]

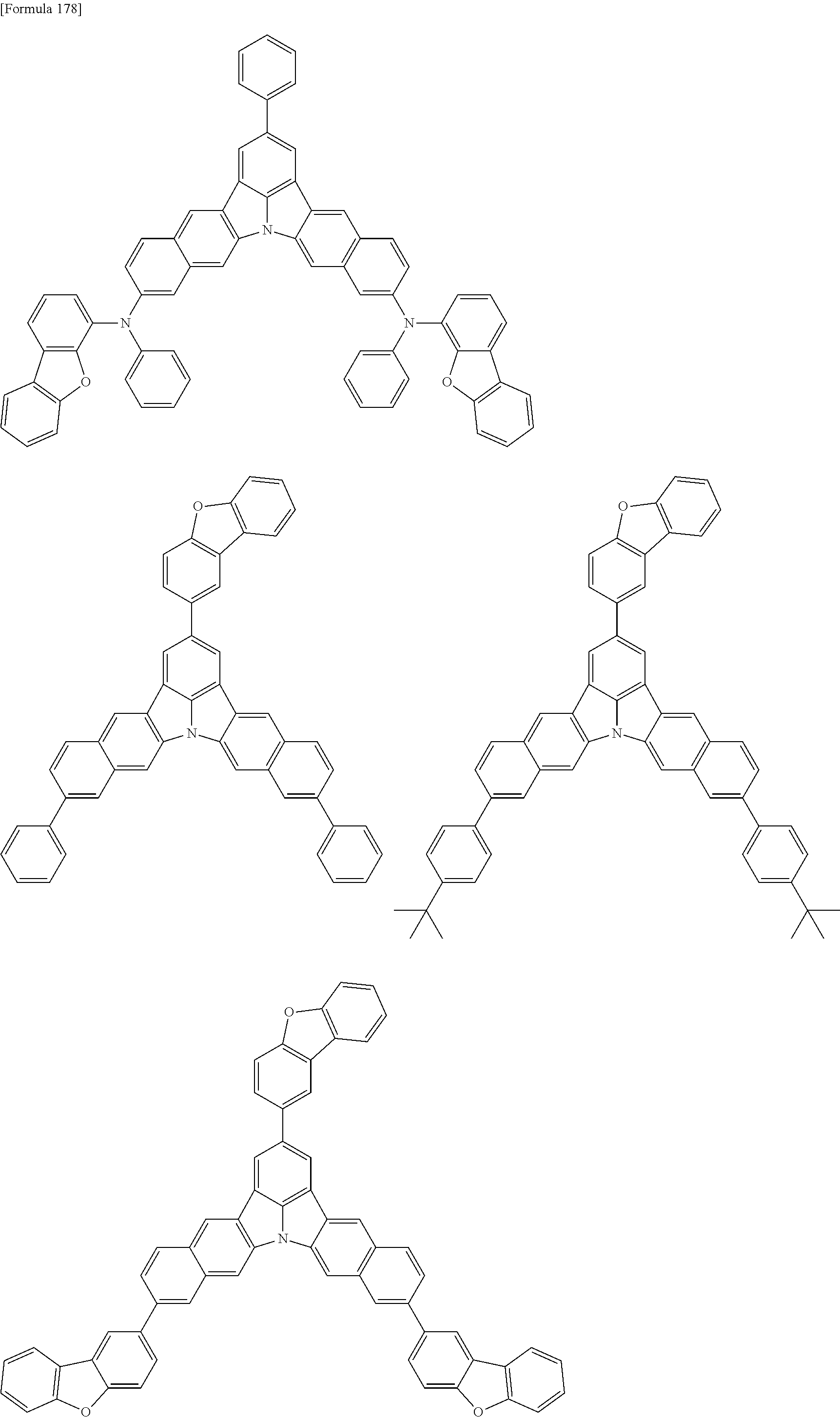

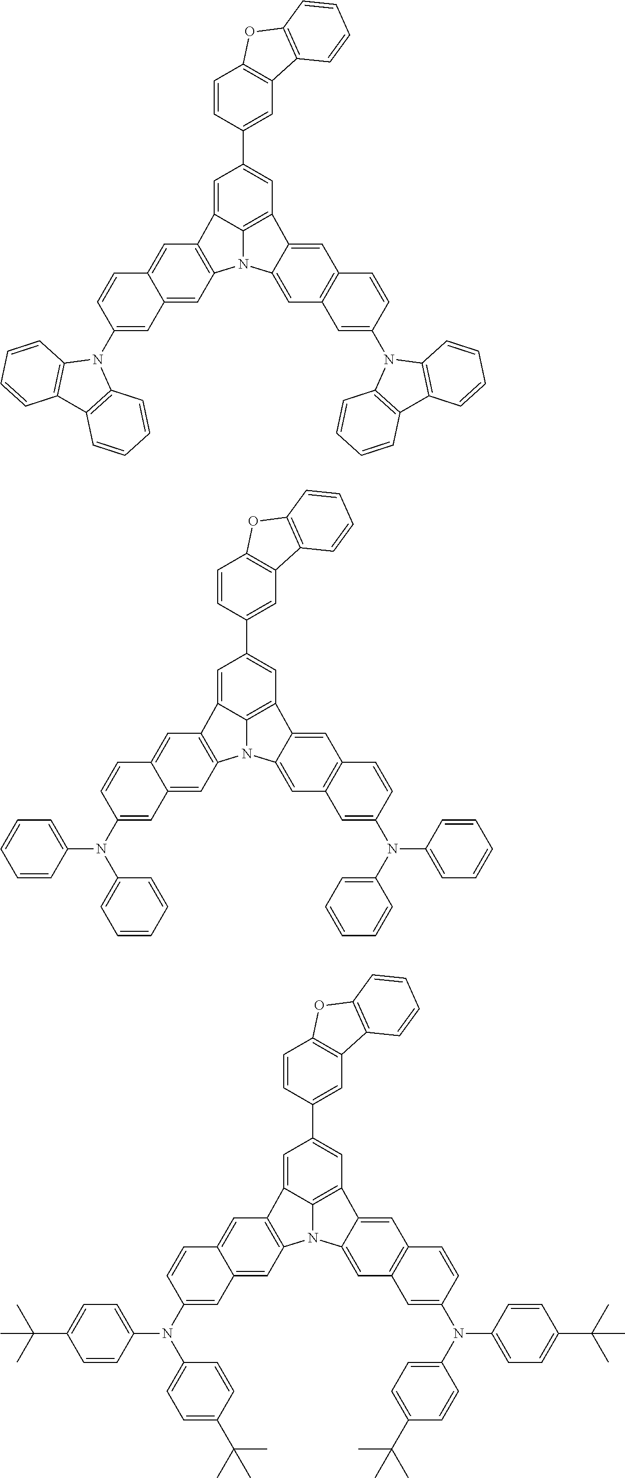

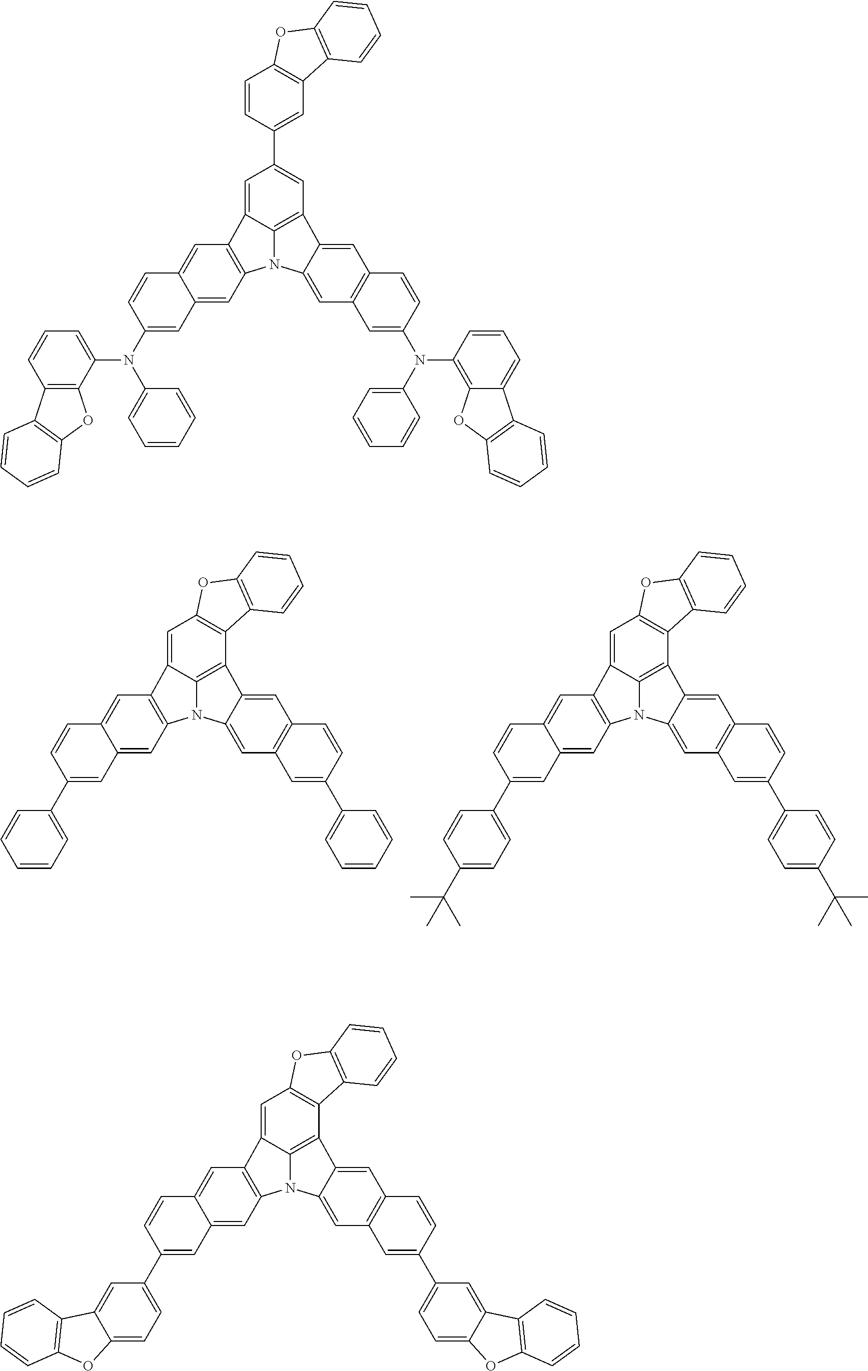

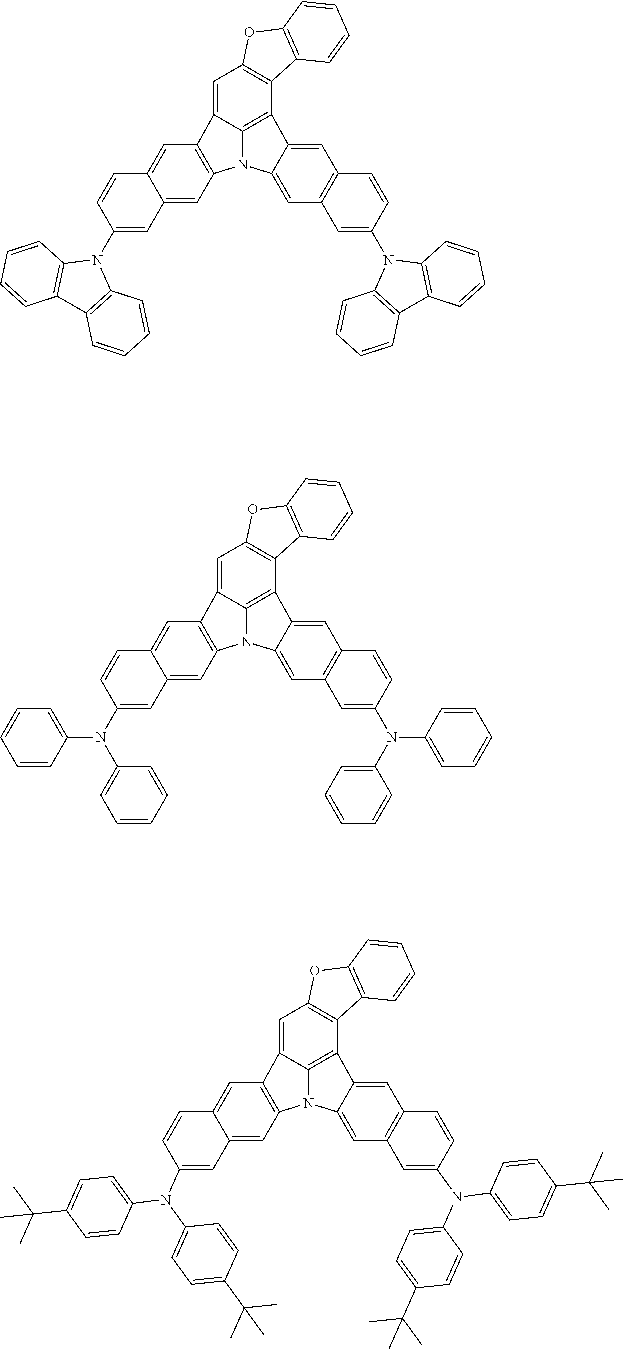 [SMILES: c1ccc2c(c1)oc1cc3c4cc5ccc(-n6c7ccccc7c7ccccc76)cc5cc4n4c5cc6cc(-n7c8ccccc8c8ccccc87)ccc6cc5c(c12)c34 ; c1ccc(N(c2ccccc2)c2ccc3cc4c5cc6oc7ccccc7c6c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6oc7ccccc7c6c6c7cc8ccc(N(c9ccc(C(C)(C)C)cc9)c9ccc(C(C)(C)C)cc9)cc8cc7n(c4cc3c2)c56)cc1]

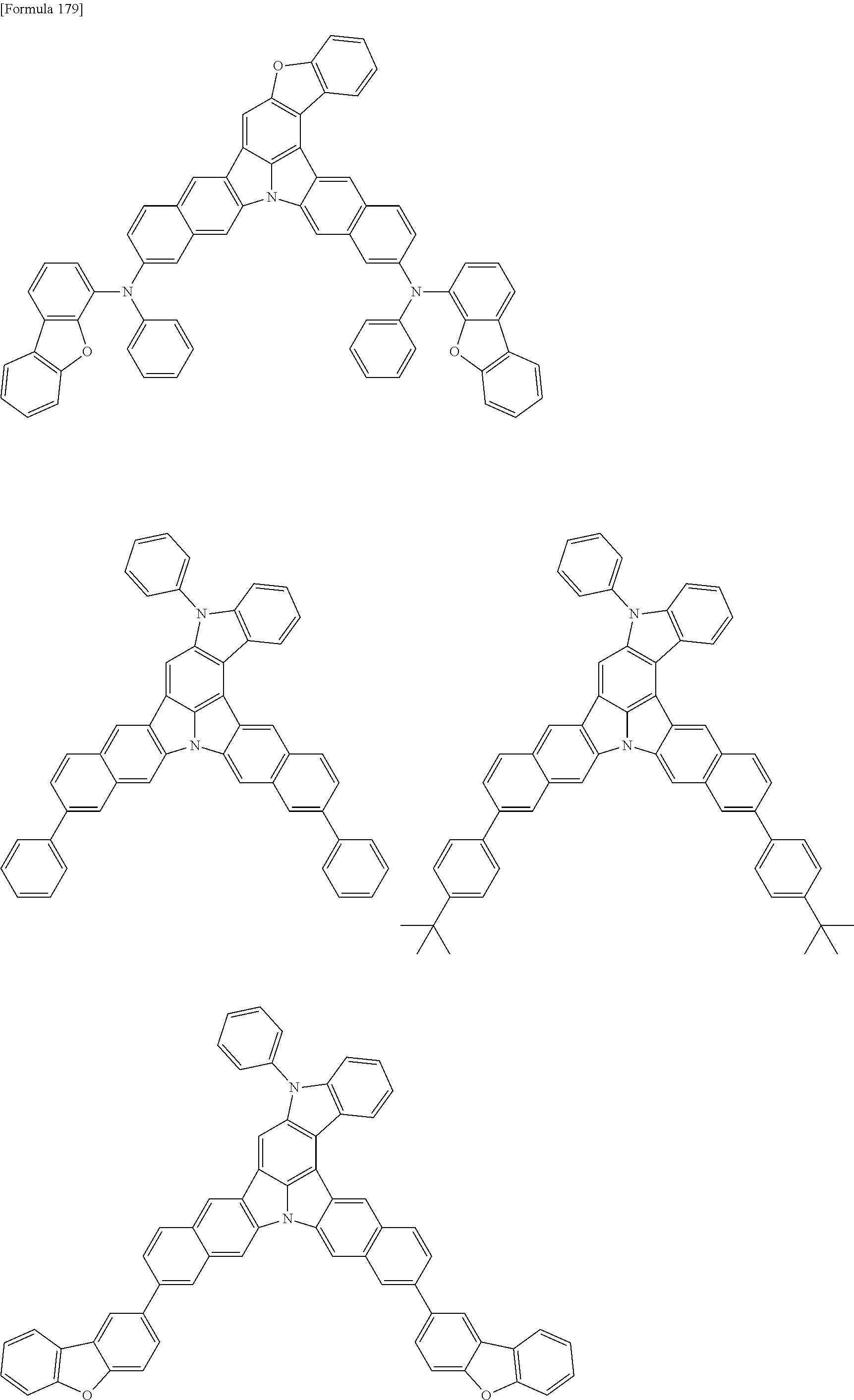

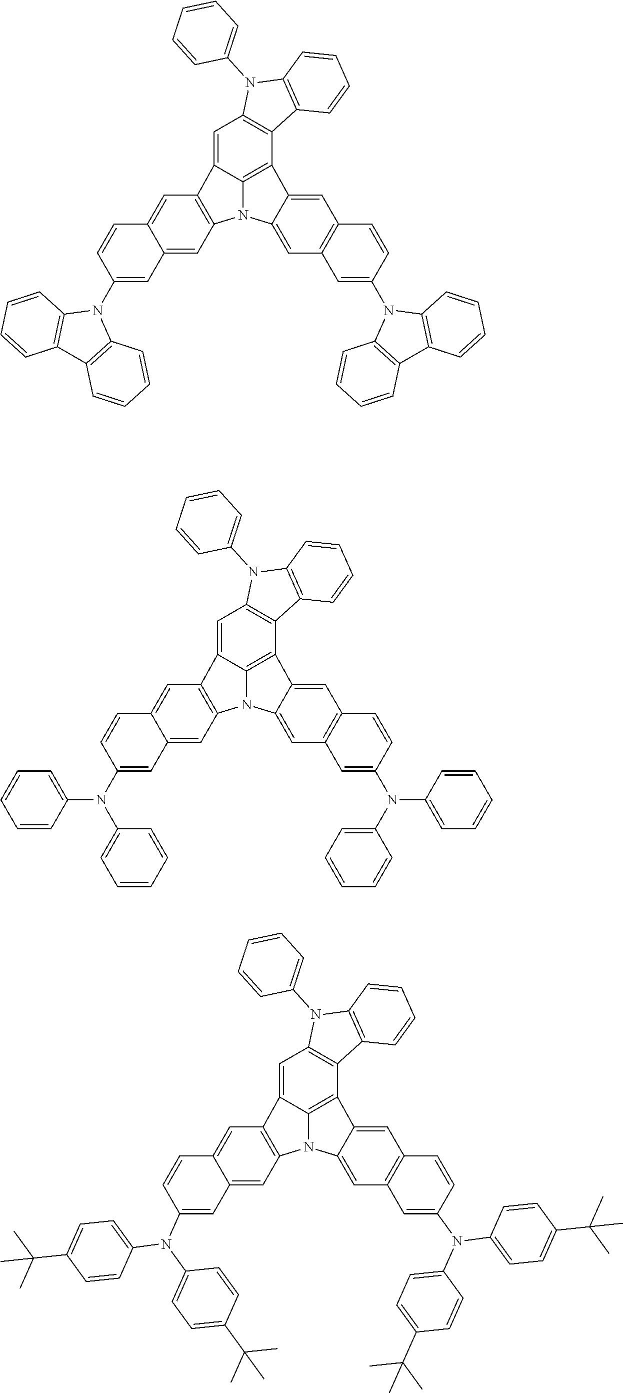 [SMILES: c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1 ; c1ccc(N(c2ccccc2)c2ccc3cc4c5cc6c(c7ccccc7n6-c6ccccc6)c6c7cc8ccc(N(c9ccccc9)c9ccccc9)cc8cc7n(c4cc3c2)c56)cc1 ; CC(C)(C)c1ccc(N(c2ccc(C(C)(C)C)cc2)c2ccc3cc4c5cc6c(c7ccccc7n6-c6ccccc6)c6c7cc8ccc(N(c9ccc(C(C)(C)C)cc9)c9ccc(C(C)(C)C)cc9)cc8cc7n(c4cc3c2)c56)cc1]

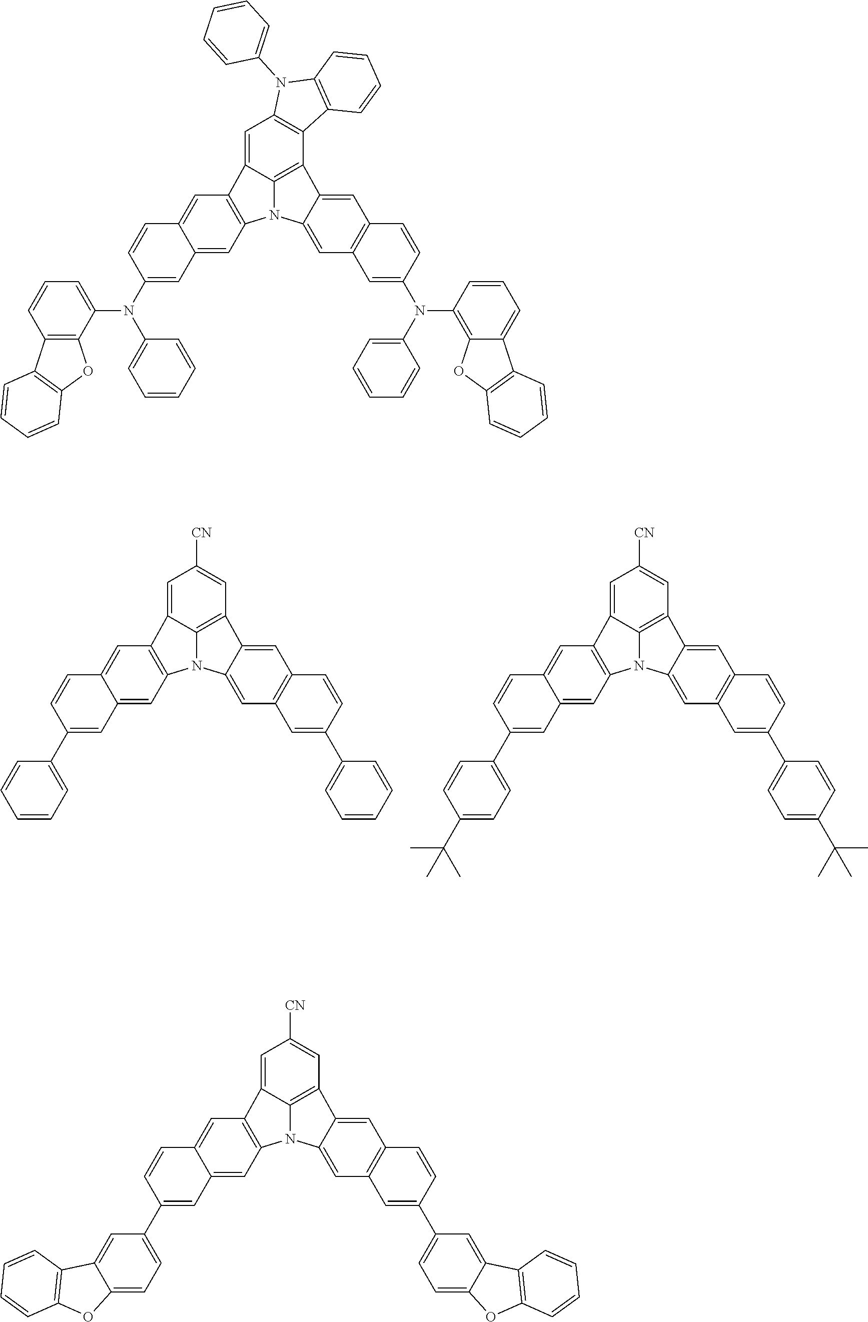

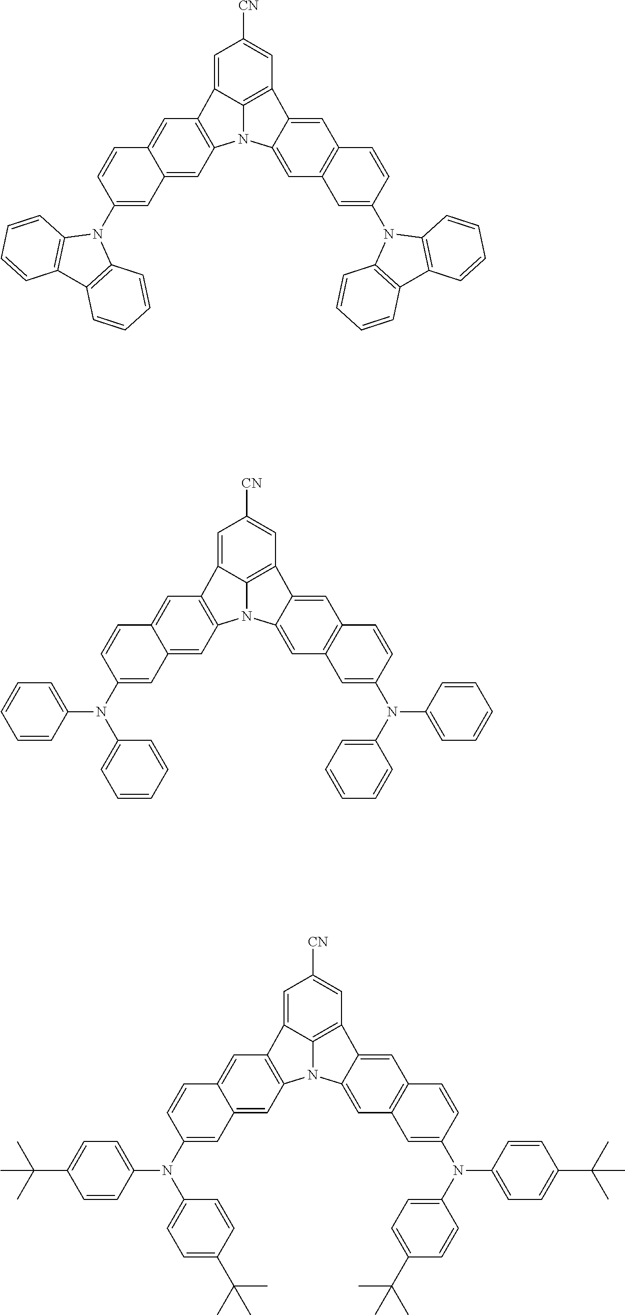 [SMILES: N#Cc1cc2c3cc4ccc(-n5c6ccccc6c6ccccc65)cc4cc3n3c4cc5cc(-n6c7ccccc7c7ccccc76)ccc5cc4c(c1)c23 ; N#Cc1cc2c3cc4ccc(N(c5ccccc5)c5ccccc5)cc4cc3n3c4cc5cc(N(c6ccccc6)c6ccccc6)ccc5cc4c(c1)c23 ; CC(C)(C)c1ccc(N(c2ccc(C(C)(C)C)cc2)c2ccc3cc4c5cc(C#N)cc6c7cc8ccc(N(c9ccc(C(C)(C)C)cc9)c9ccc(C(C)(C)C)cc9)cc8cc7n(c4cc3c2)c56)cc1]

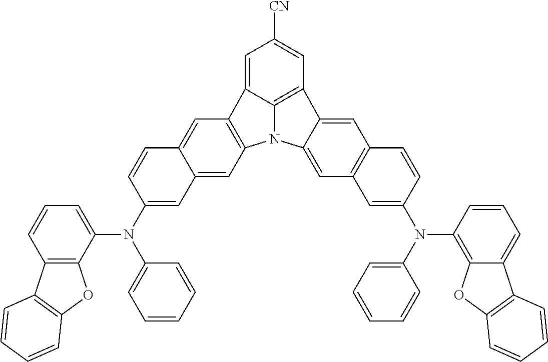 [SMILES: N#Cc1cc2c3cc4ccc(N(c5ccccc5)c5cccc6c5oc5ccccc56)cc4cc3n3c4cc5cc(N(c6ccccc6)c6cccc7c6oc6ccccc67)ccc5cc4c(c1)c23]

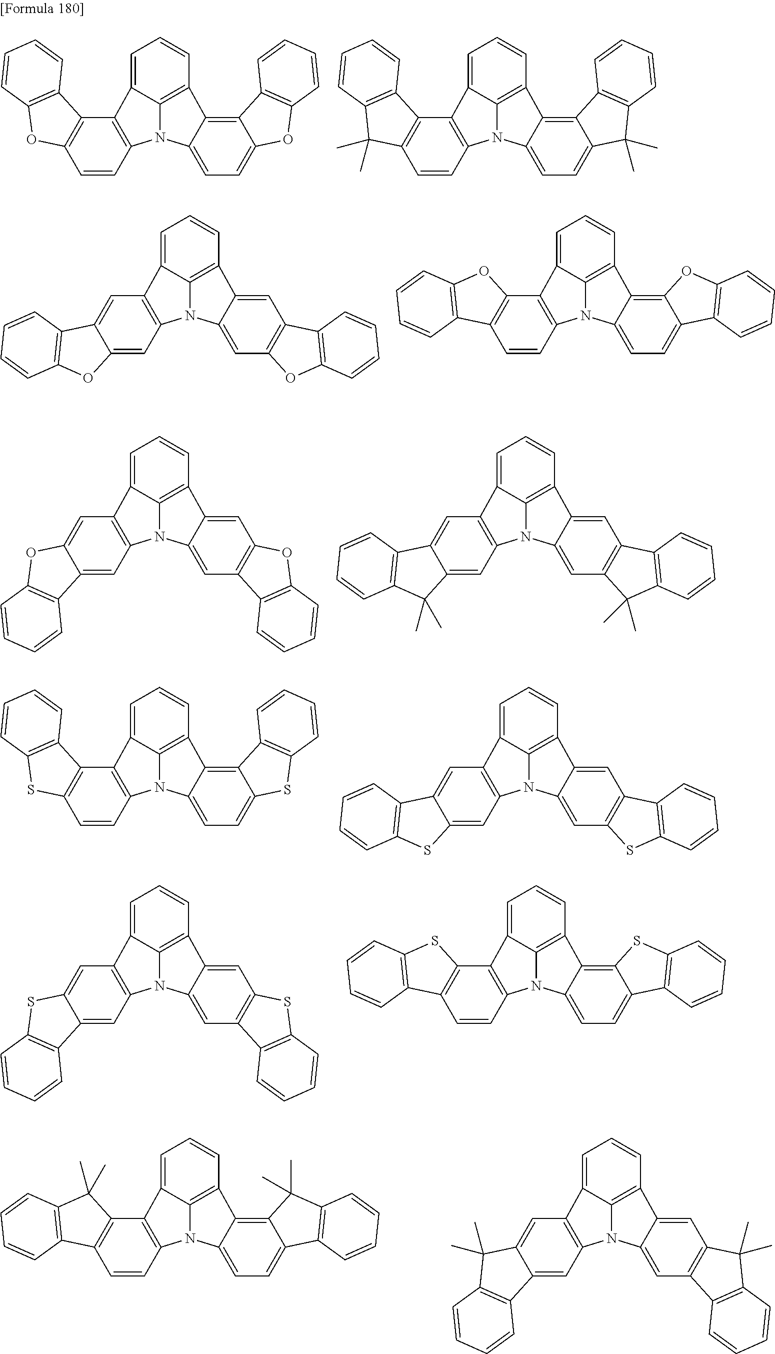

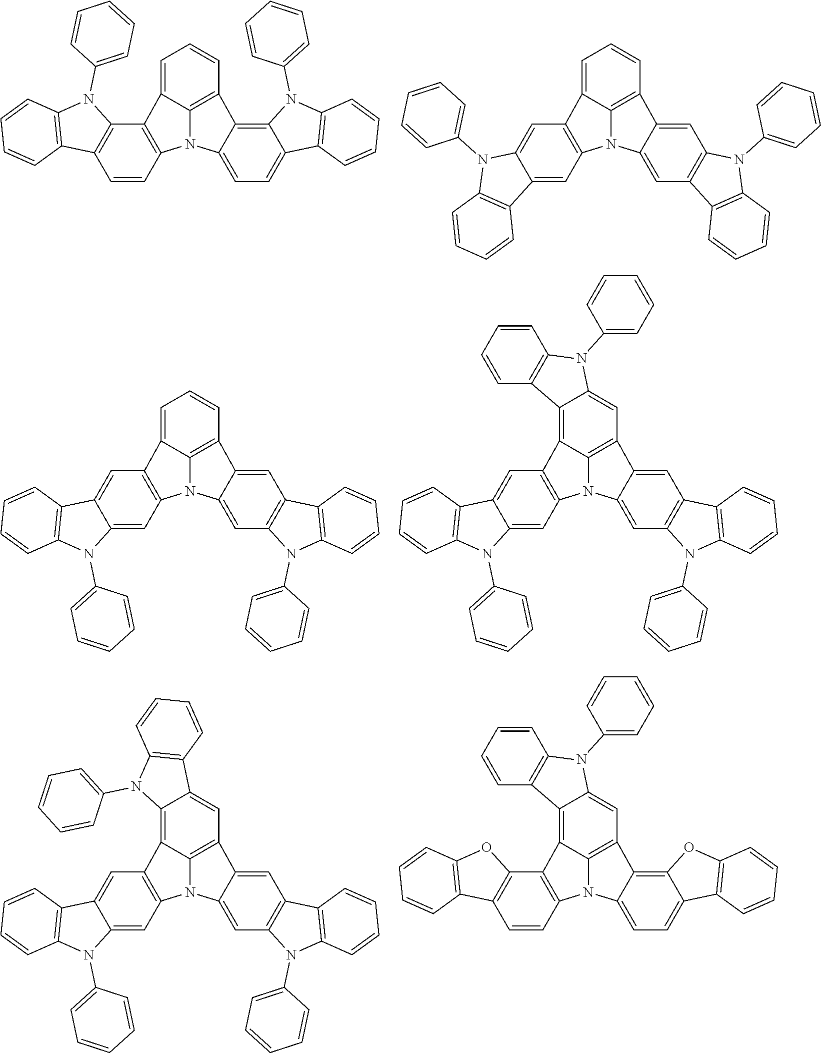

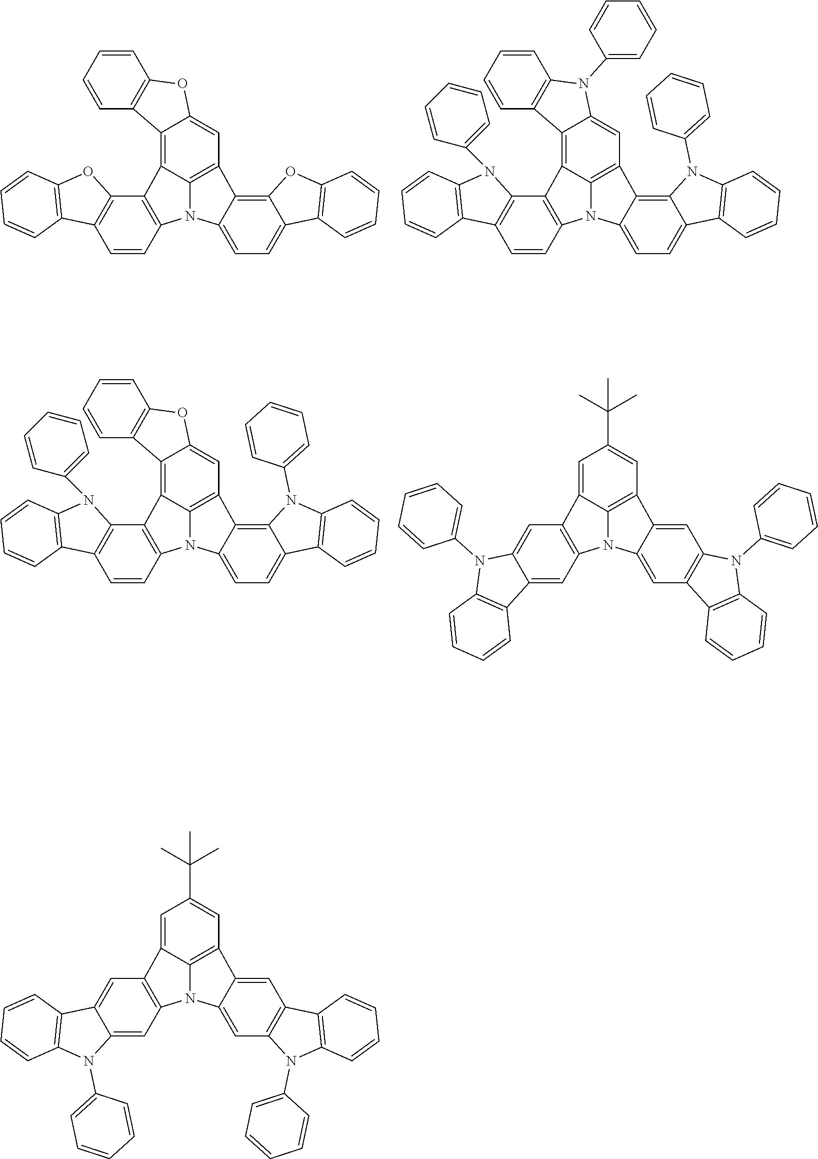

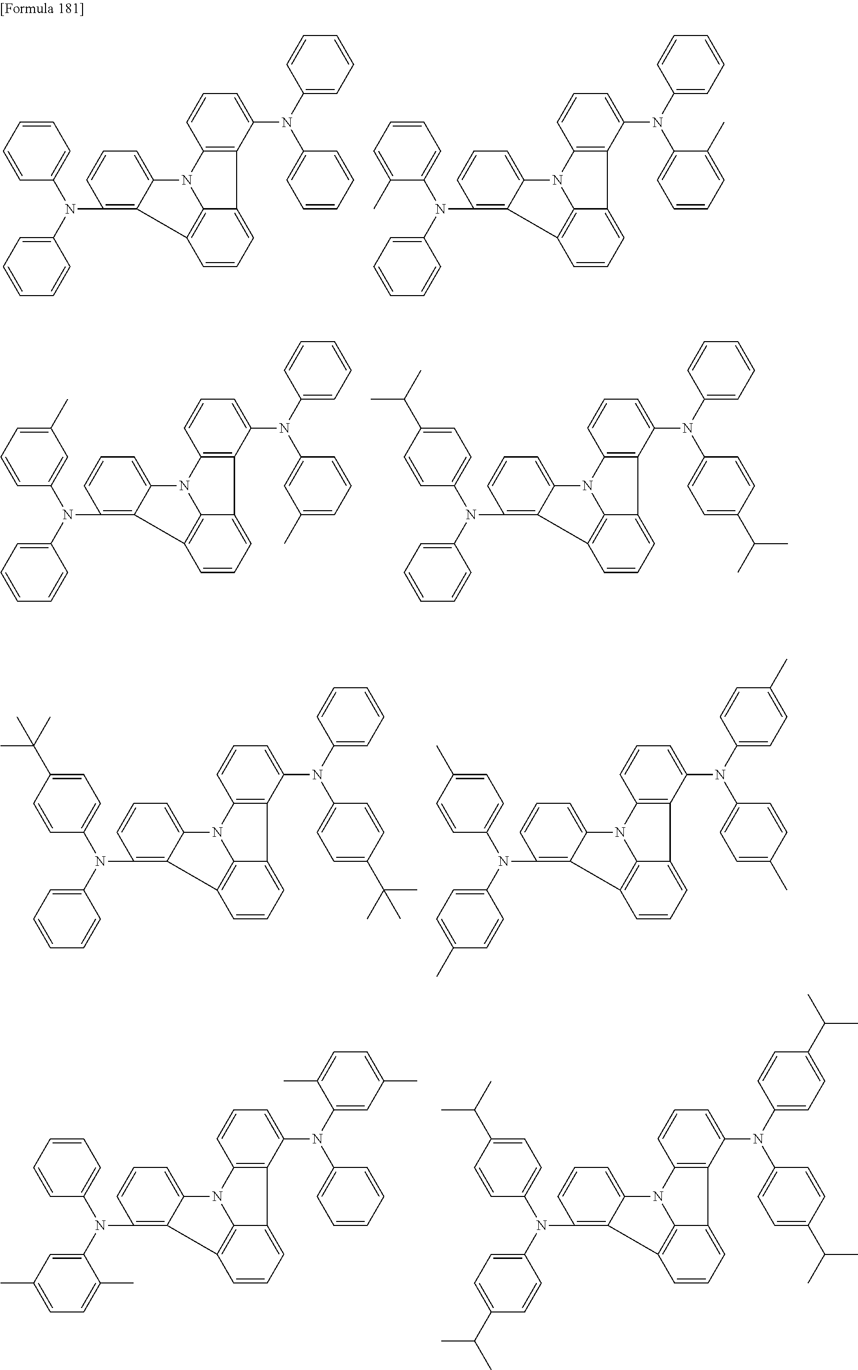

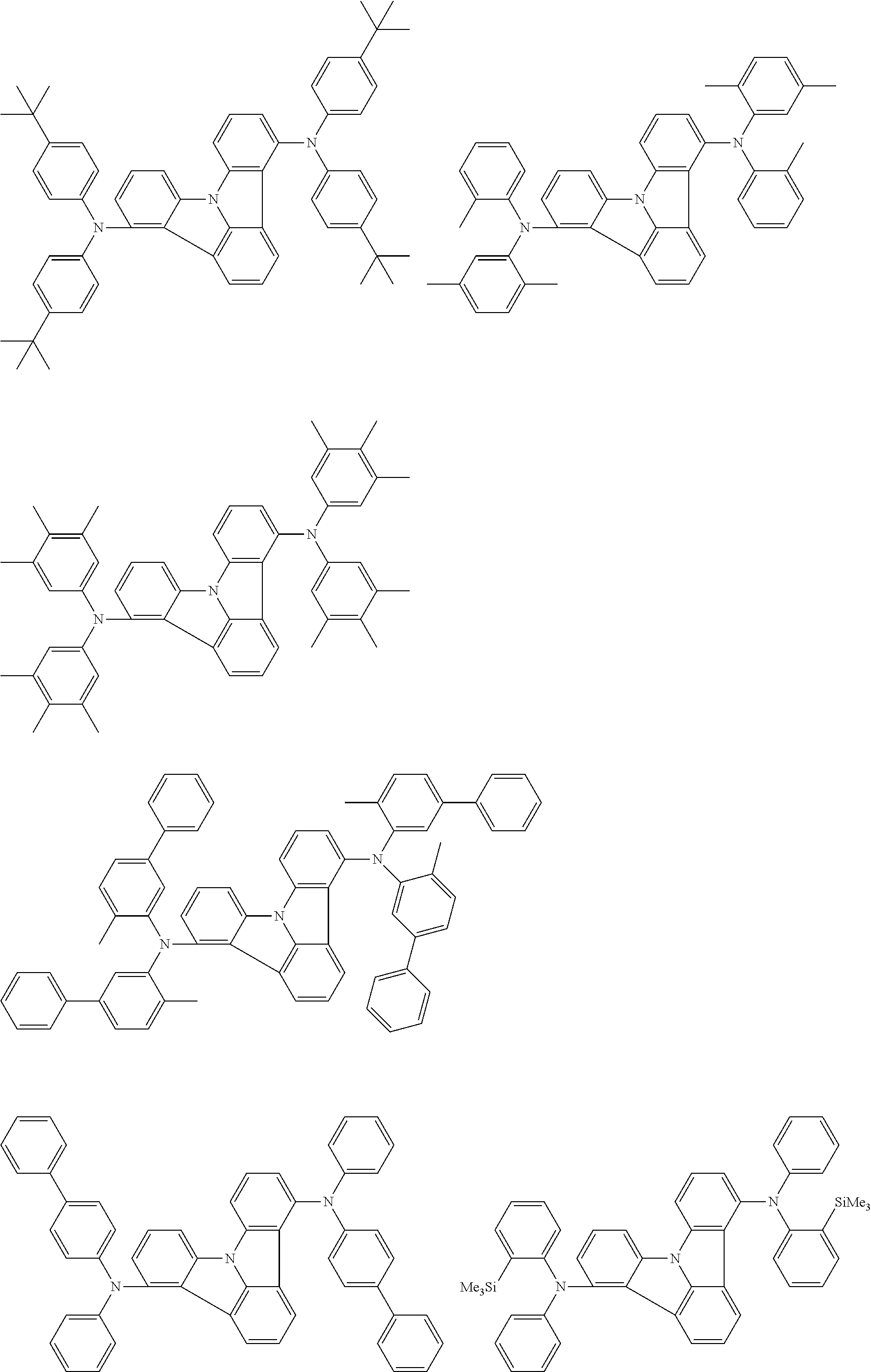

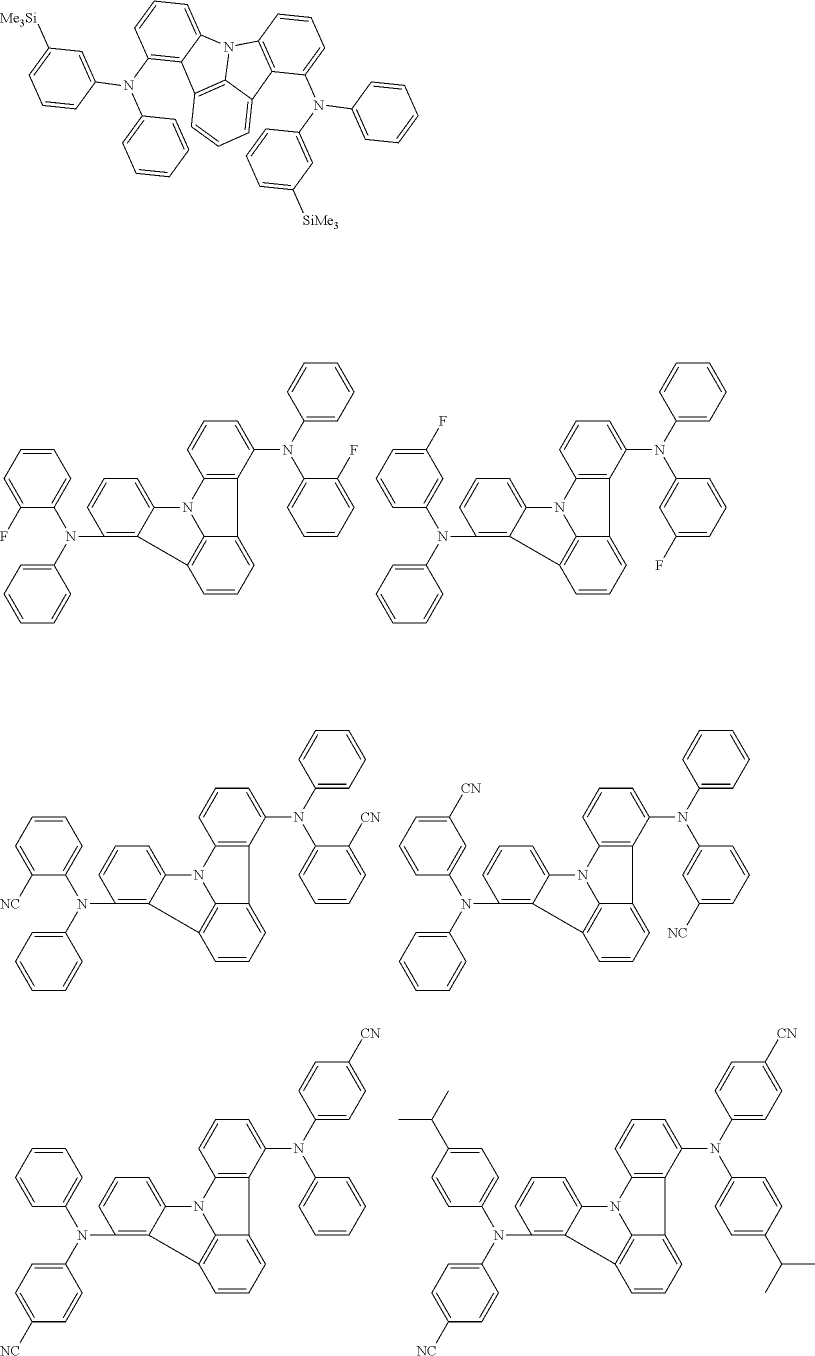

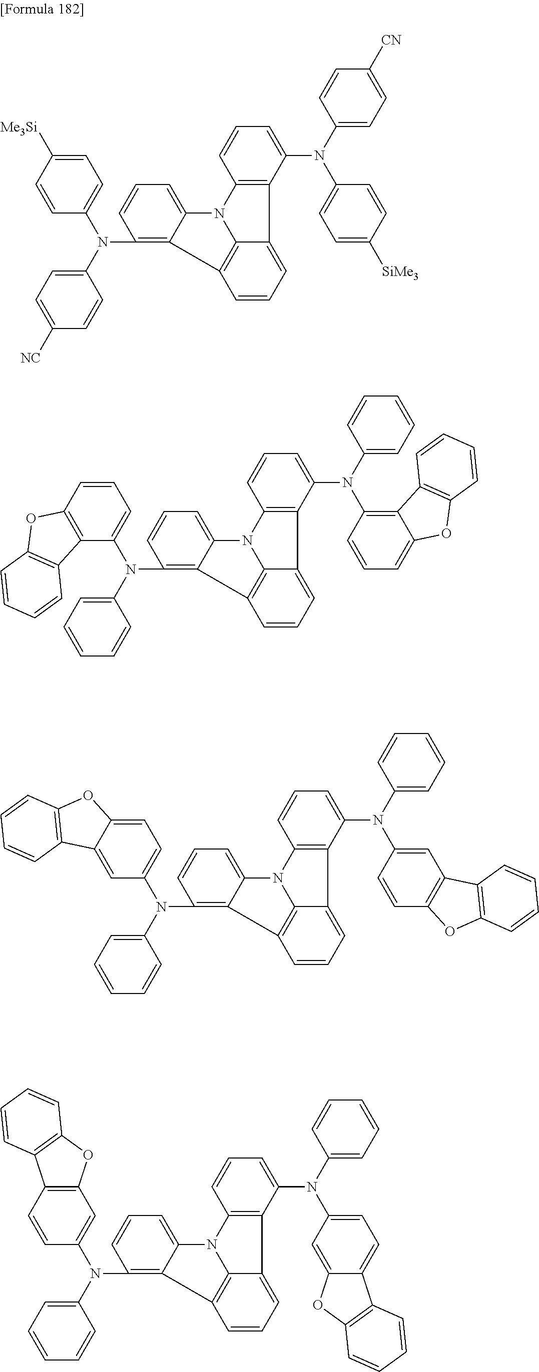 [SMILES: C[Si](C)(C)c1ccc(N(c2ccc(C#N)cc2)c2cccc3c2c2cccc4c5c(N(c6ccc(C#N)cc6)c6ccc([Si](C)(C)C)cc6)cccc5n3c24)cc1 ; c1ccc(N(c2cccc3oc4ccccc4c23)c2cccc3c2c2cccc4c5c(N(c6ccccc6)c6cccc7oc8ccccc8c67)cccc5n3c24)cc1 ; c1ccc(N(c2ccc3oc4ccccc4c3c2)c2cccc3c2c2cccc4c5c(N(c6ccccc6)c6ccc7oc8ccccc8c7c6)cccc5n3c24)cc1 ; c1ccc(N(c2ccc3c(c2)oc2ccccc23)c2cccc3c2c2cccc4c5c(N(c6ccccc6)c6ccc7c(c6)oc6ccccc67)cccc5n3c24)cc1]

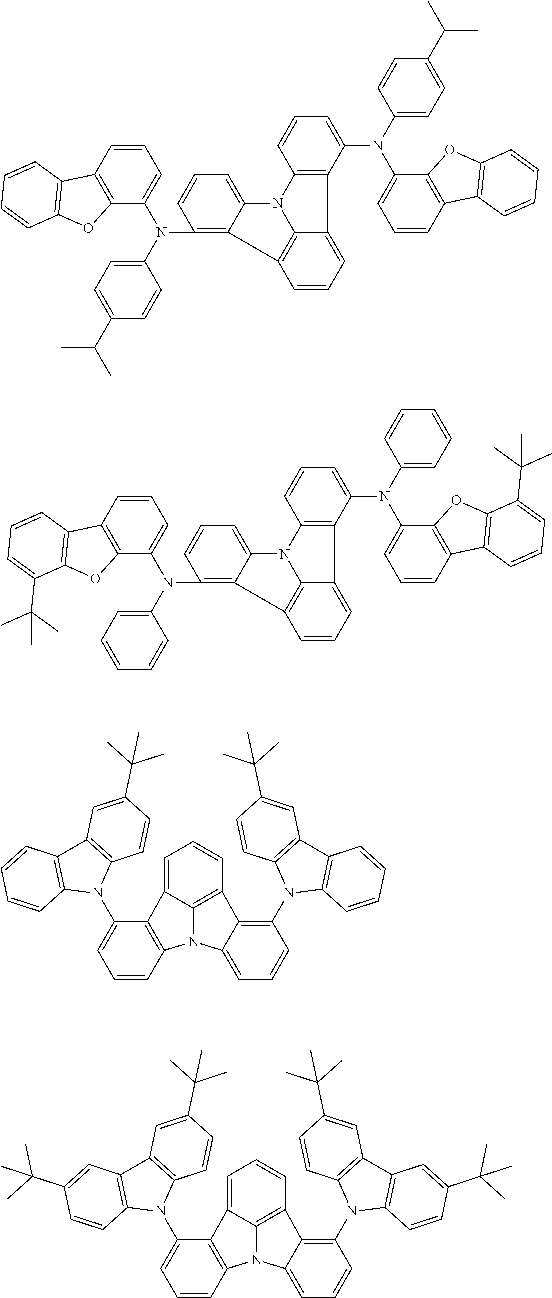

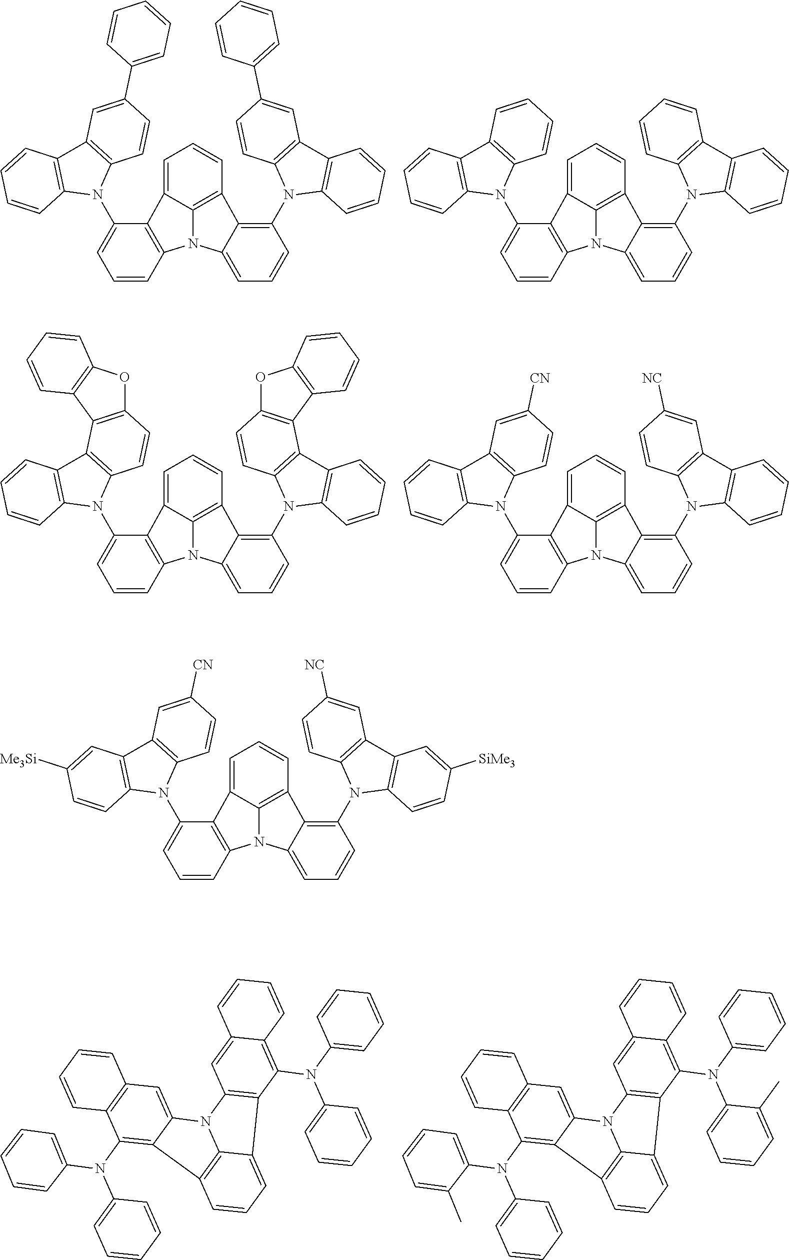

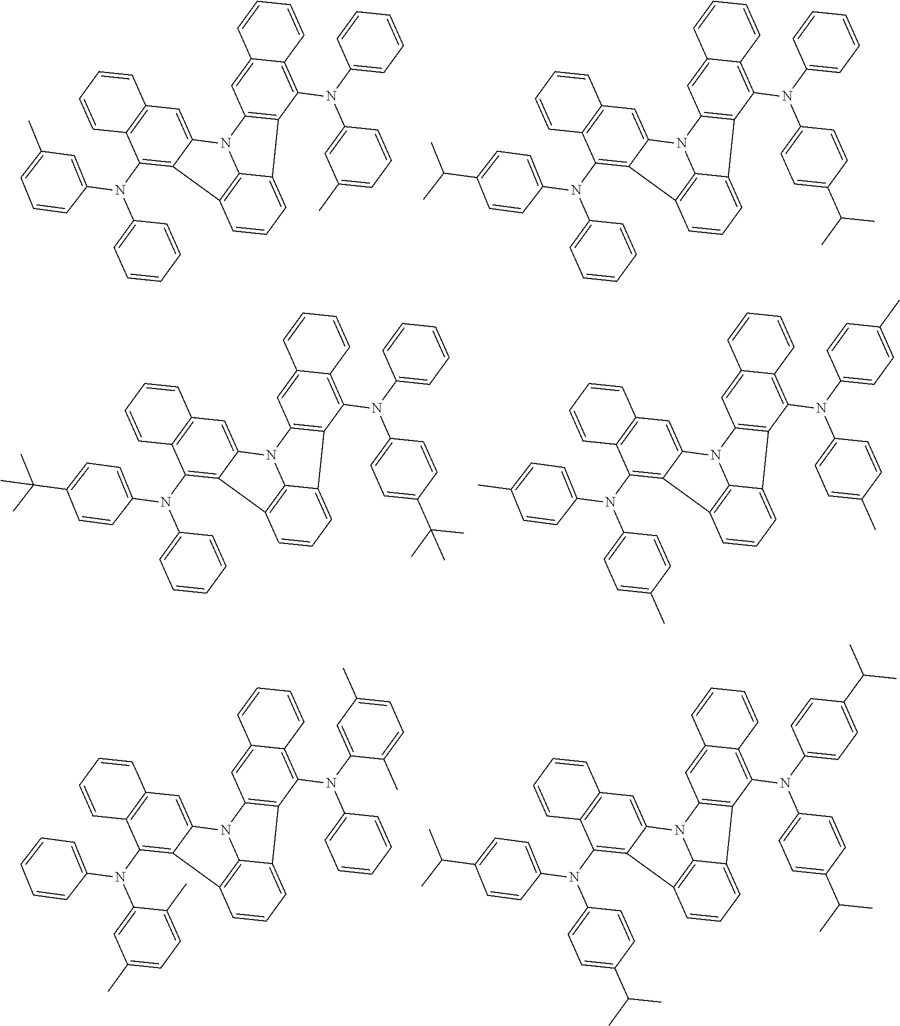

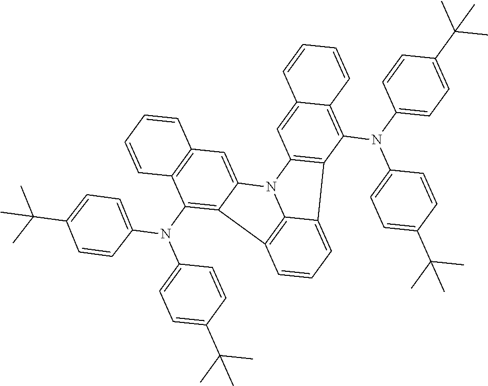 [SMILES: CC(C)(C)c1ccc(N(c2ccc(C(C)(C)C)cc2)c2c3ccccc3cc3c2c2cccc4c5c(N(c6ccc(C(C)(C)C)cc6)c6ccc(C(C)(C)C)cc6)c6ccccc6cc5n3c24)cc1]

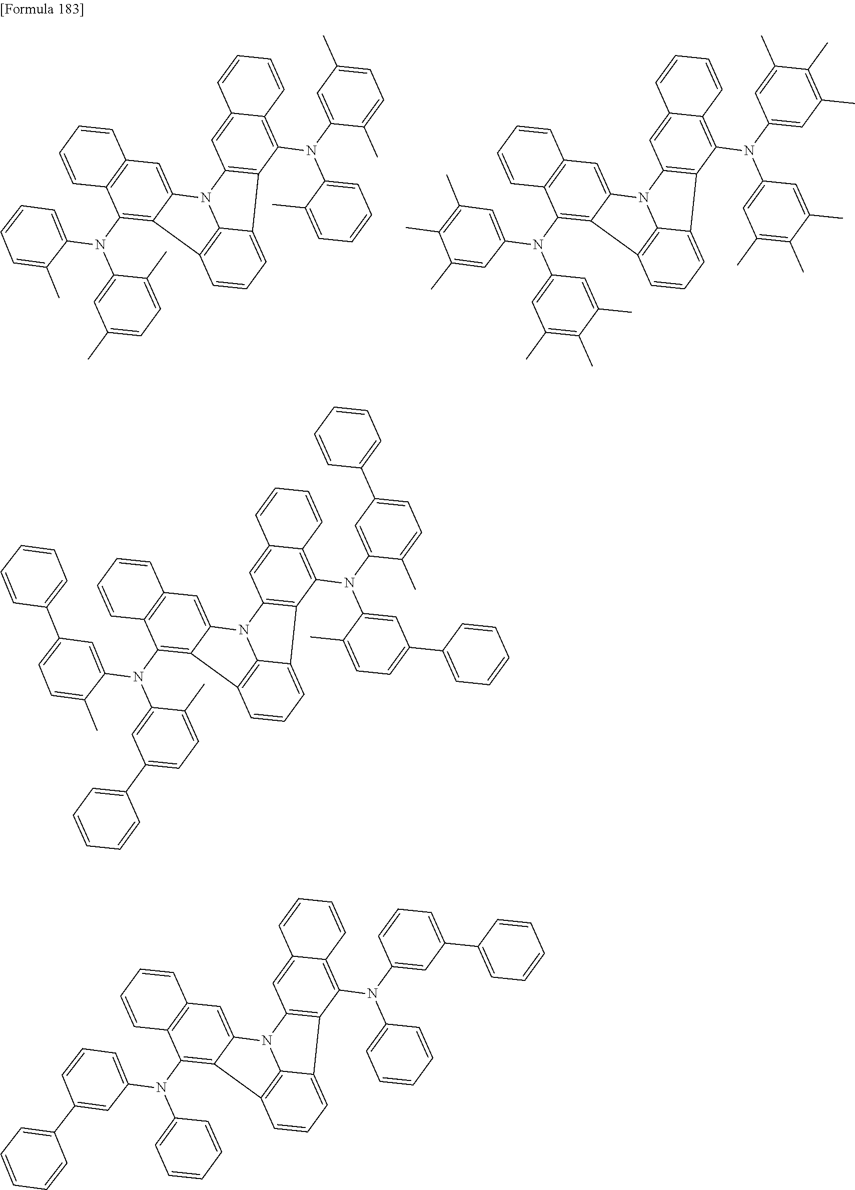

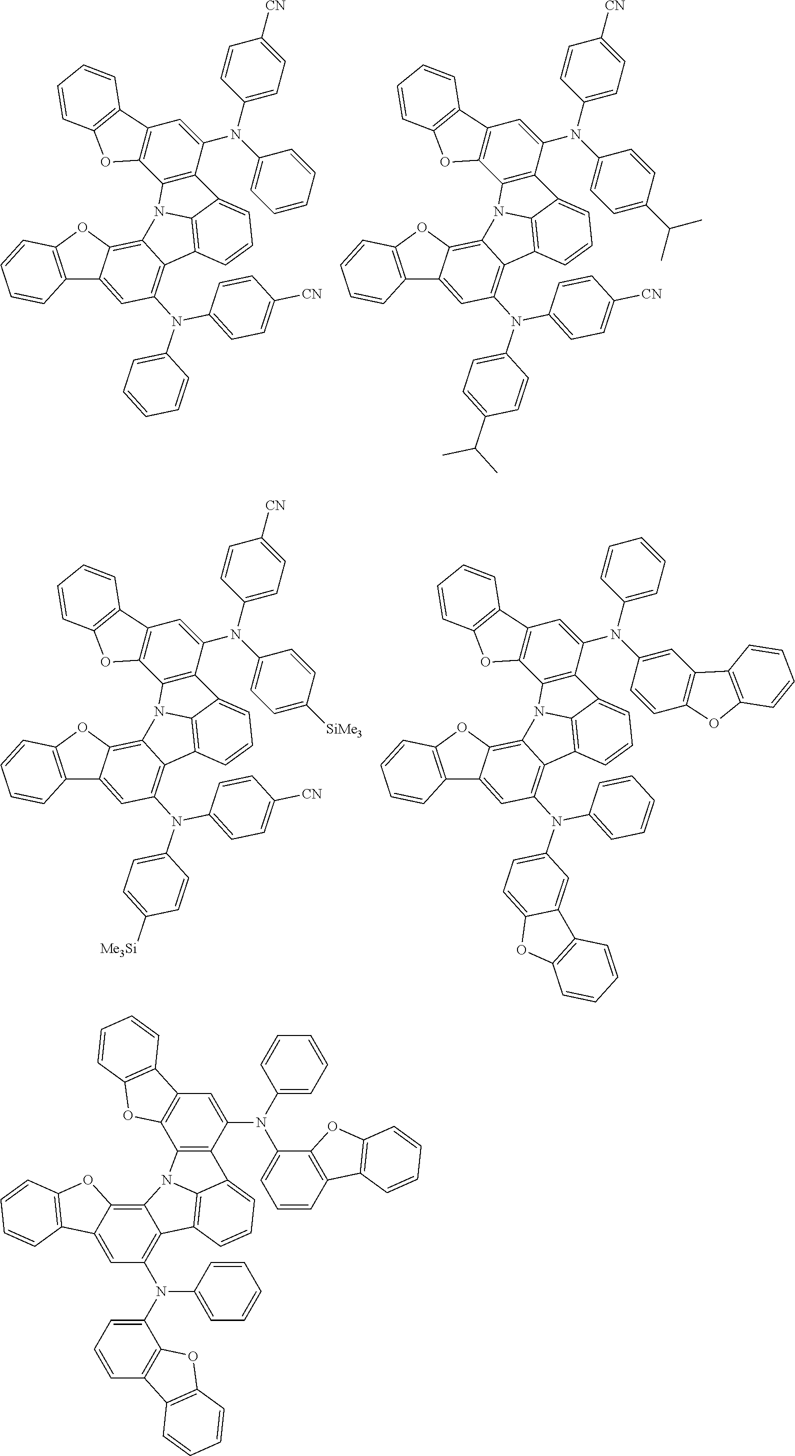

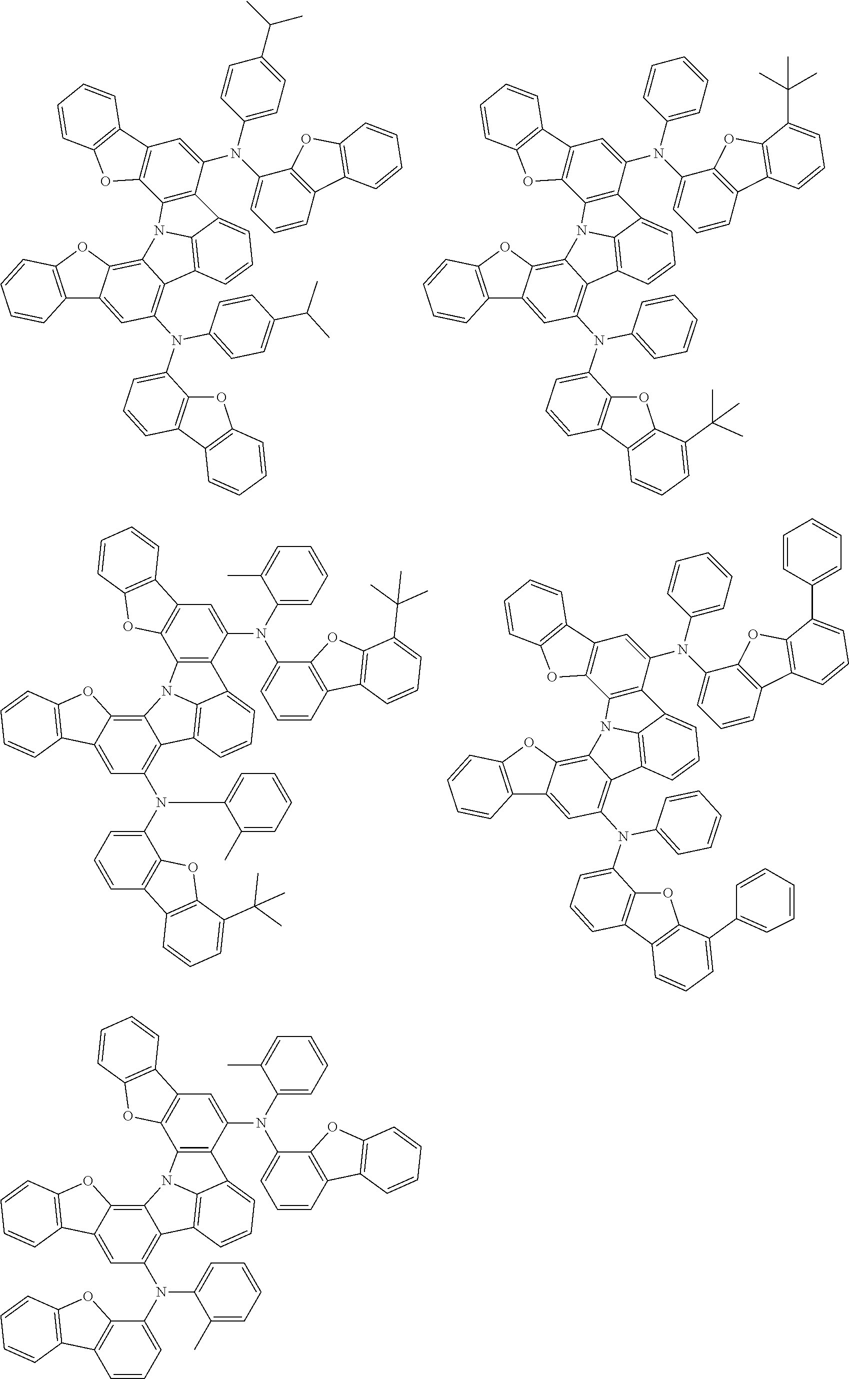

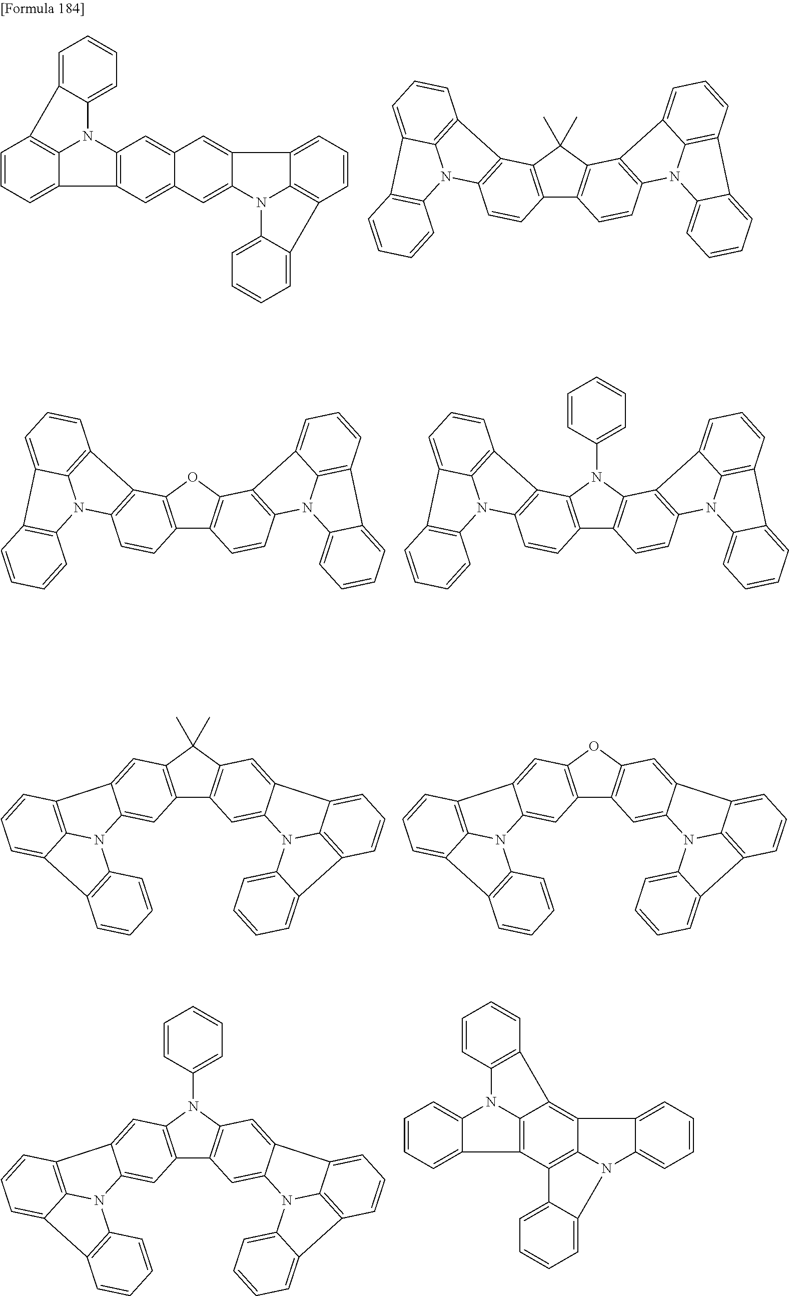

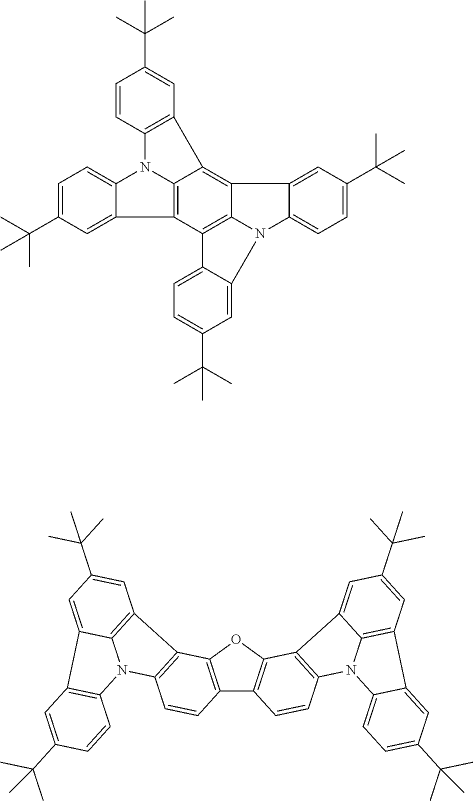

### Compound Represented by Formula (5)

The compound represented by the formula (5) will be described. The compound represented by the formula (5) corresponds to the compound represented by the above-described formula (41-3).

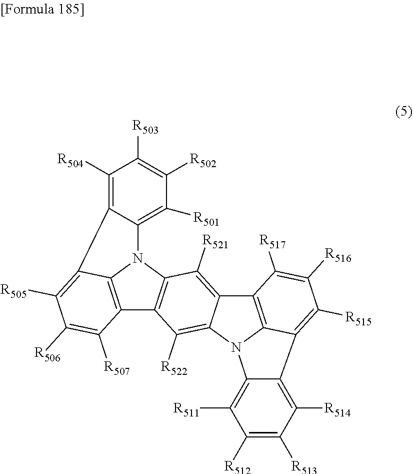

In the formula (5):

at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded;

R₅₀₁ to R₅₀₇ and R₅₁₁ to R₅₁₇ neither forming the monocyclic ring nor forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and

R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

“A combination of adjacent two or more of R₅₀₁ to R₅₀₇ and R₅₁₁ to R₅₁₇′ refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, at least one, preferably two of R₅₀₁ to R₅₀₇ and R₅₁₁ to R₅₁₇ are groups represented by —N(R₉₀₆)(R₉₀₇).

In an exemplary embodiment, R₅₀₁ to R₅₀₇ and R₅₁₁ to R₅₁₇ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (52) below.

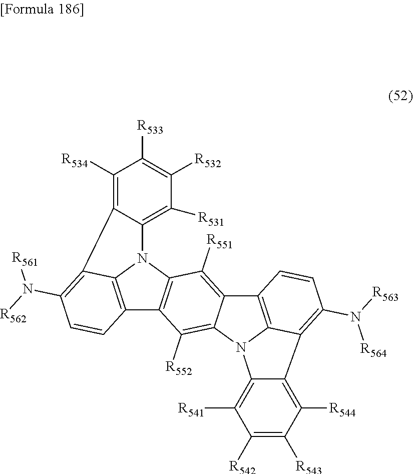

In the formula (52):

at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;

R₅₃₁ to R₅₃₄, R₅₄₁ to R₅₄₄ forming neither the monocyclic ring nor the fused ring, and R₅₅₁ and R₅₅₂ are each independently a hydrogen atom, a substituted or unsubstituted aryl group having 6 to 50 ring carbon atoms, or a substituted or unsubstituted heterocyclic group having 5 to 50 ring atoms; and

R₅₆₁ to R₅₆₄ are each independently a substituted or unsubstituted aryl group having 6 to 50 ring carbon atoms, or a substituted or unsubstituted heterocyclic group having 5 to 50 ring atoms.

In an exemplary embodiment, the compound represented by the formula (5) is a compound represented by a formula (53) below.

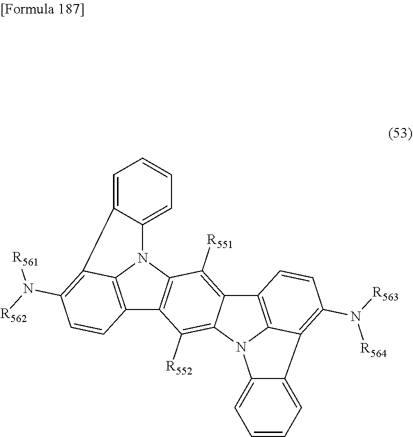

In the formula (53), R₅₅₁, R₅₅₂, and R₅₆₁ to R₅₆₄ each independently represent the same as R₅₅₁, R₅₅₂, and R₅₆₁ to R₅₆₄ in the formula (52).

In an exemplary embodiment, R₅₆₁ to R₅₆₄ in the formulae (52) and (53) are each independently a substituted or unsubstituted aryl group having 6 to 50 ring carbon atoms (preferably a phenyl group).

In an exemplary embodiment, R₅₂₁ and R₅₂₂ in the formula (5) and R₅₅₁ and R₅₅₂ in the formulae (52) and (53) are hydrogen atoms.

In an exemplary embodiment, the substituent for “substituted or unsubstituted” in the formulae (5), (52) and (53) is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

Specific examples of the compound represented by the formula (5) include compounds shown below.

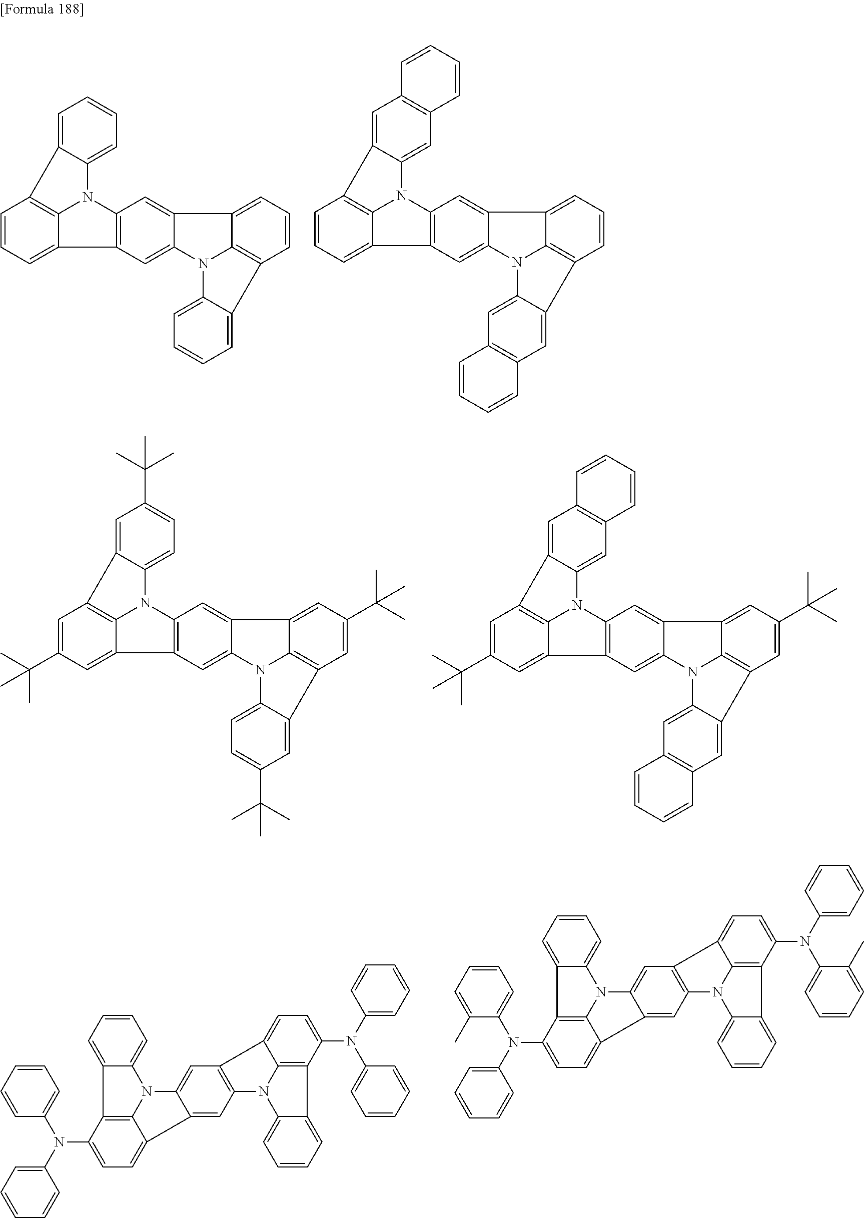

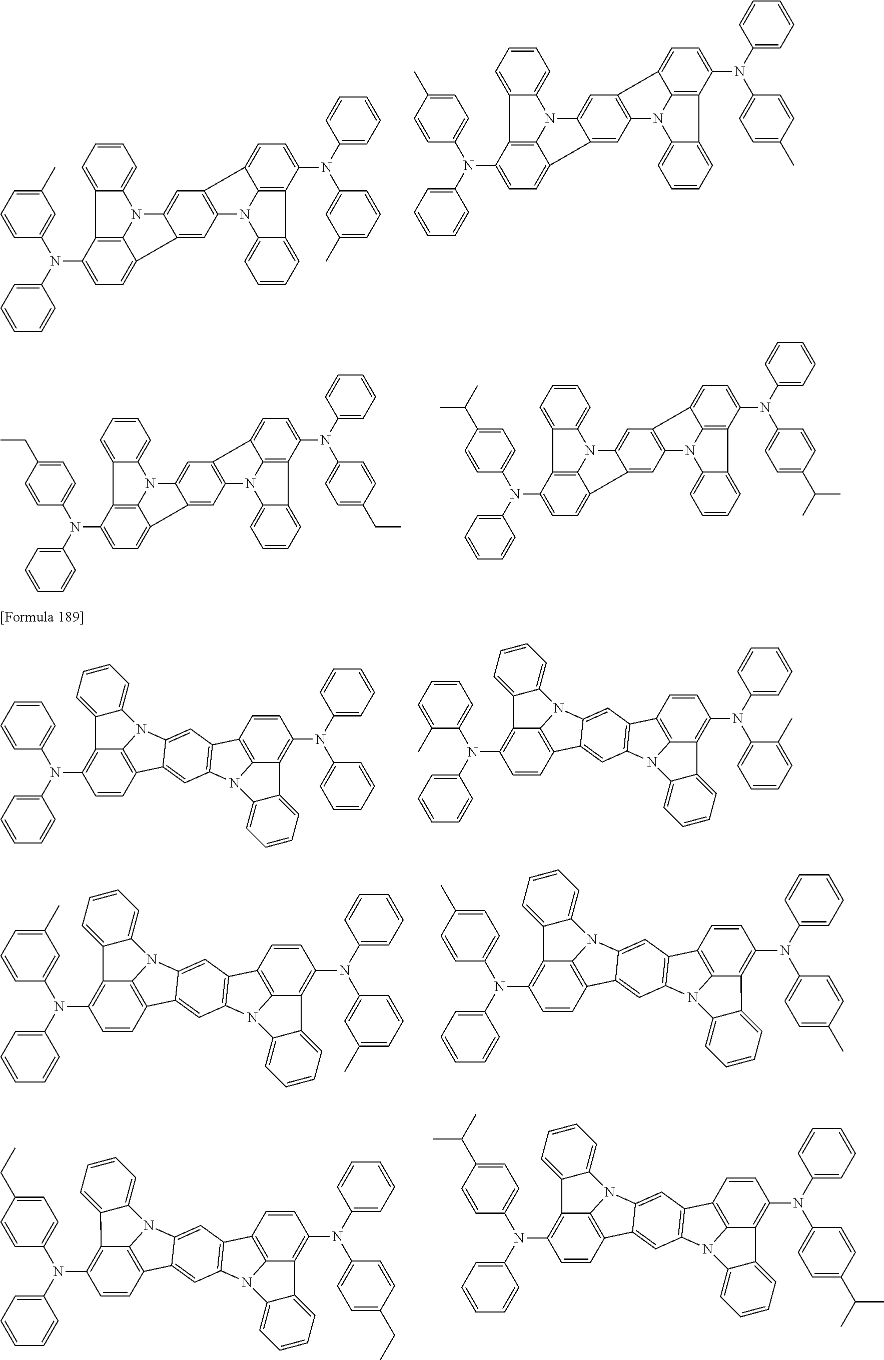

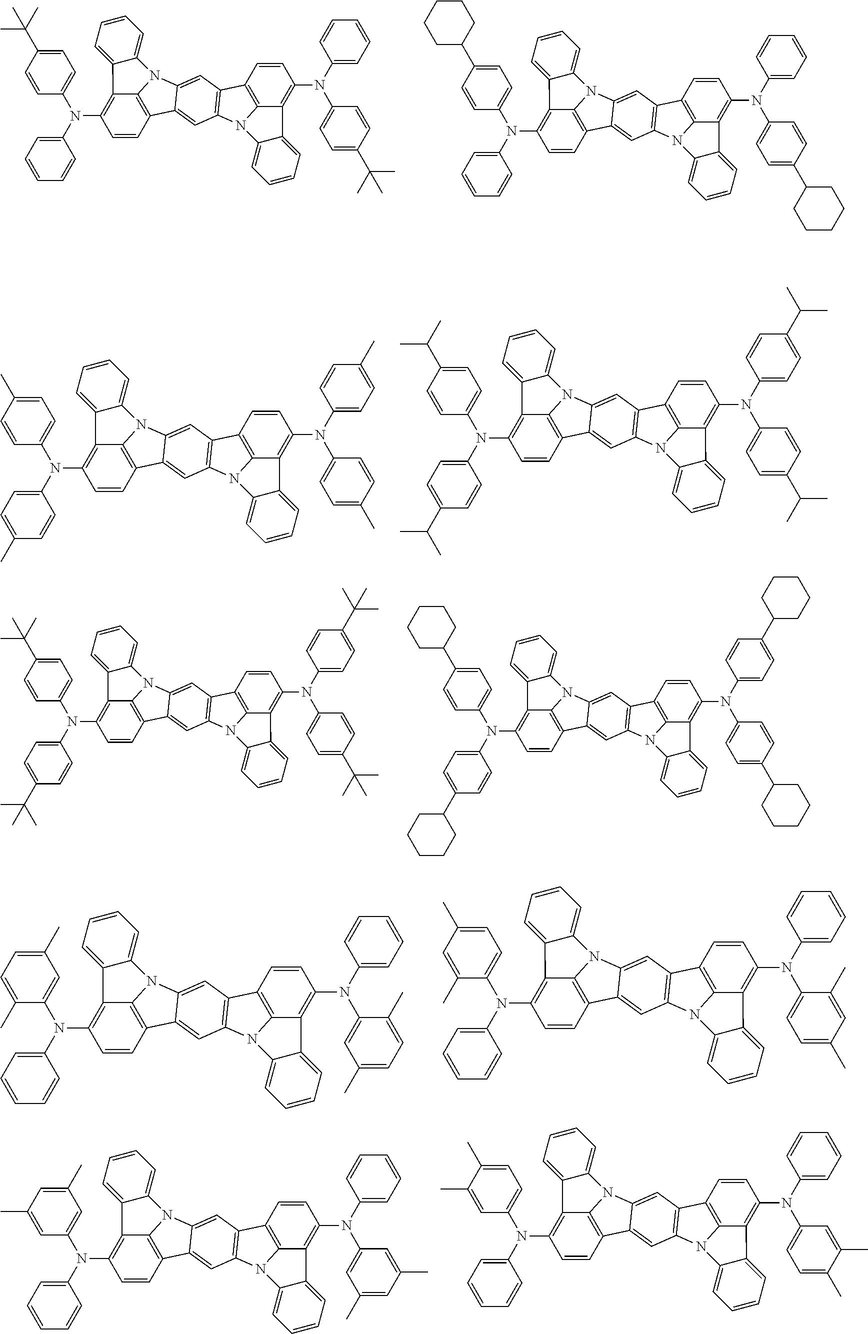

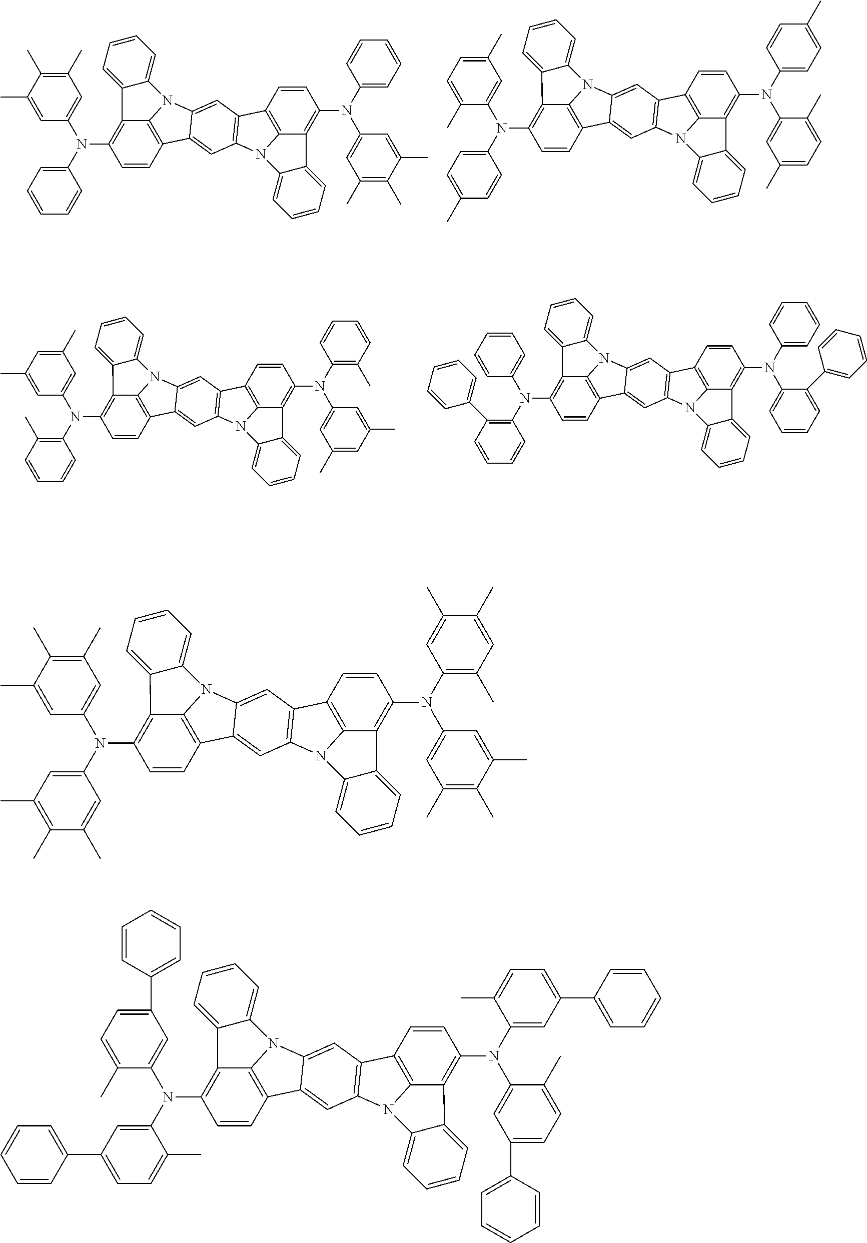

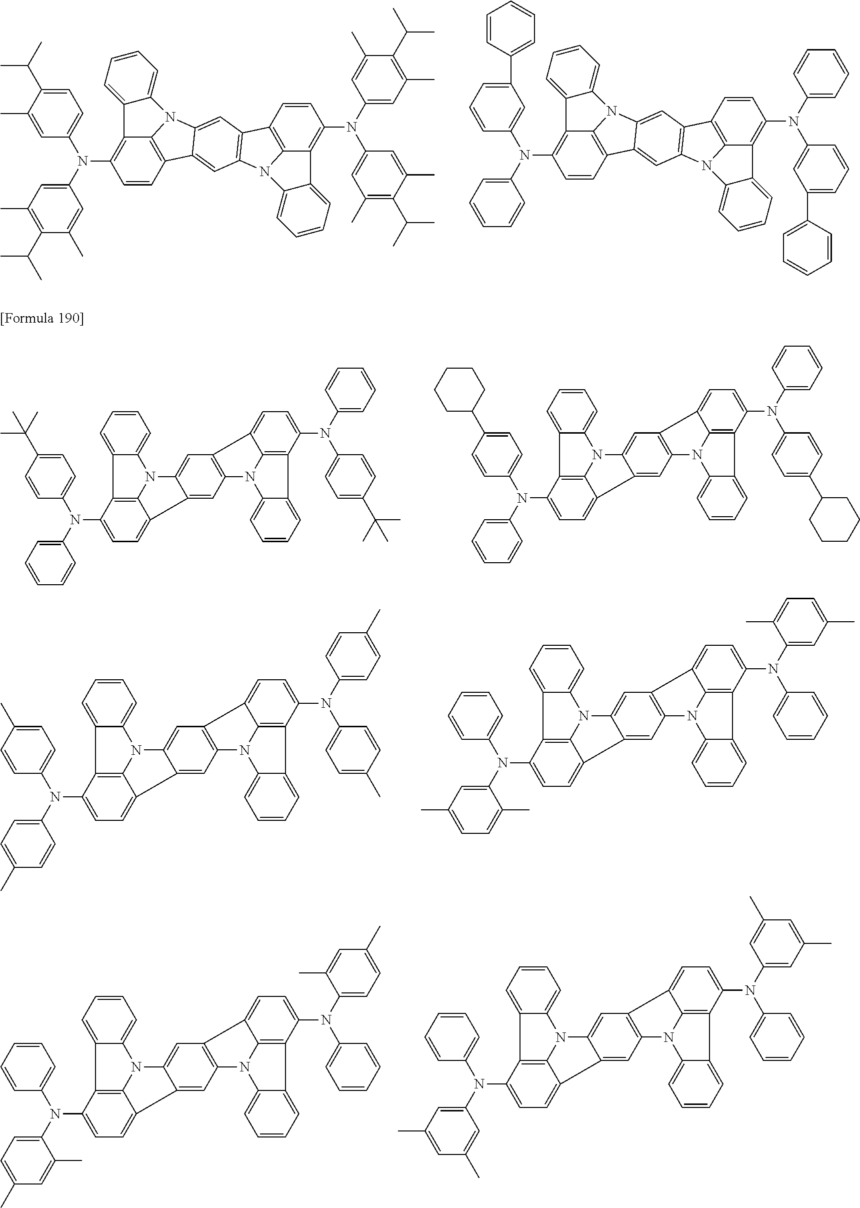

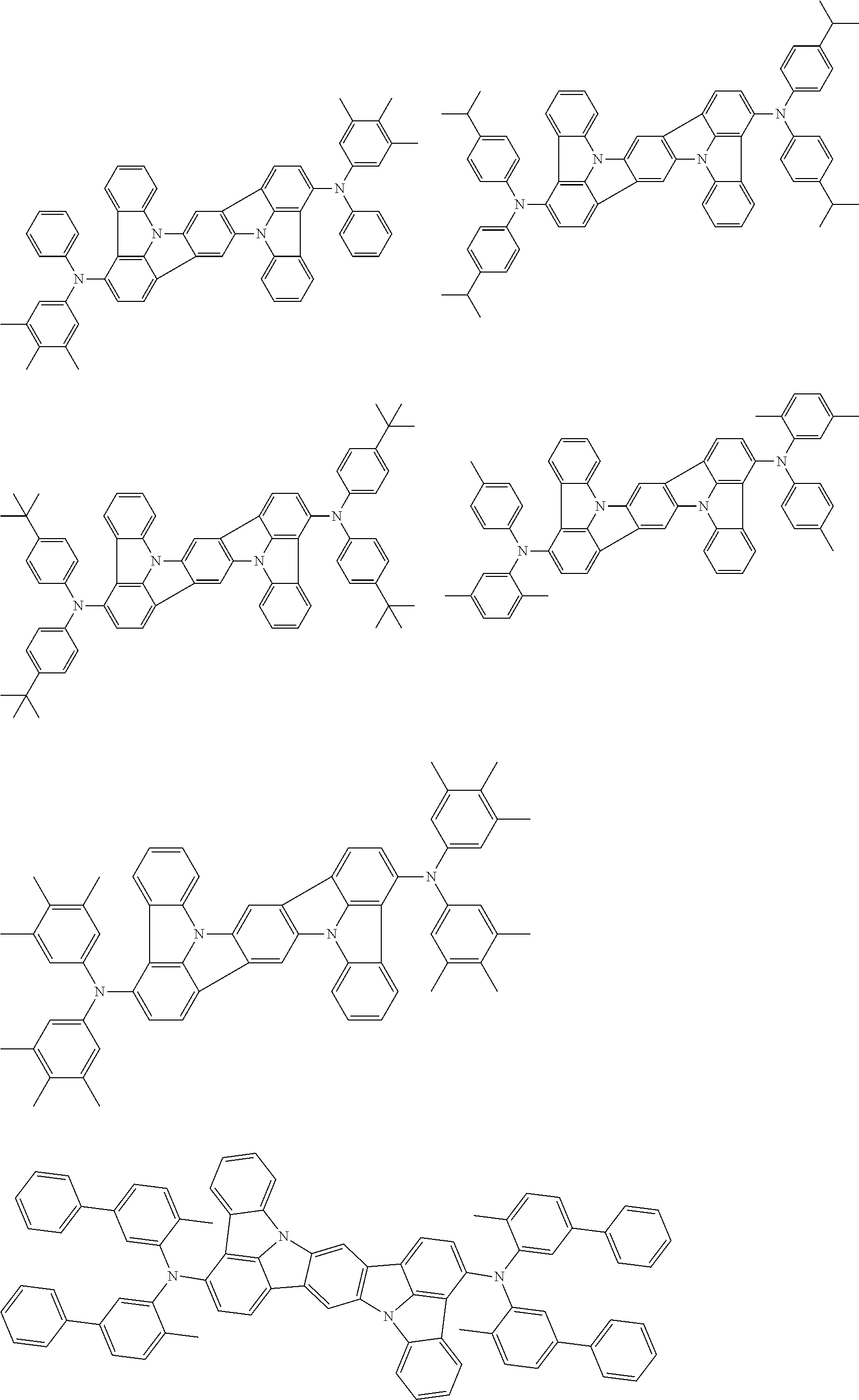

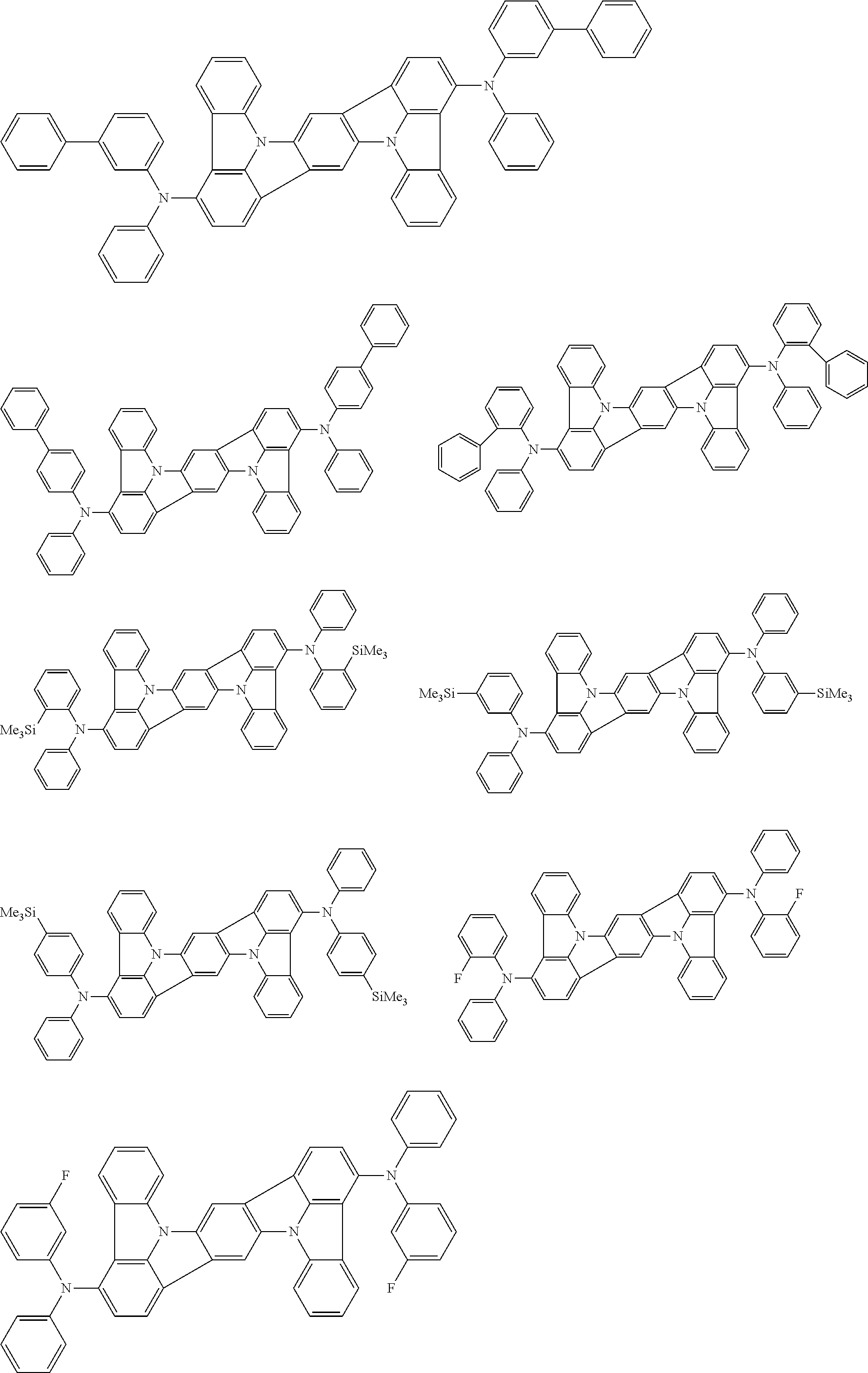

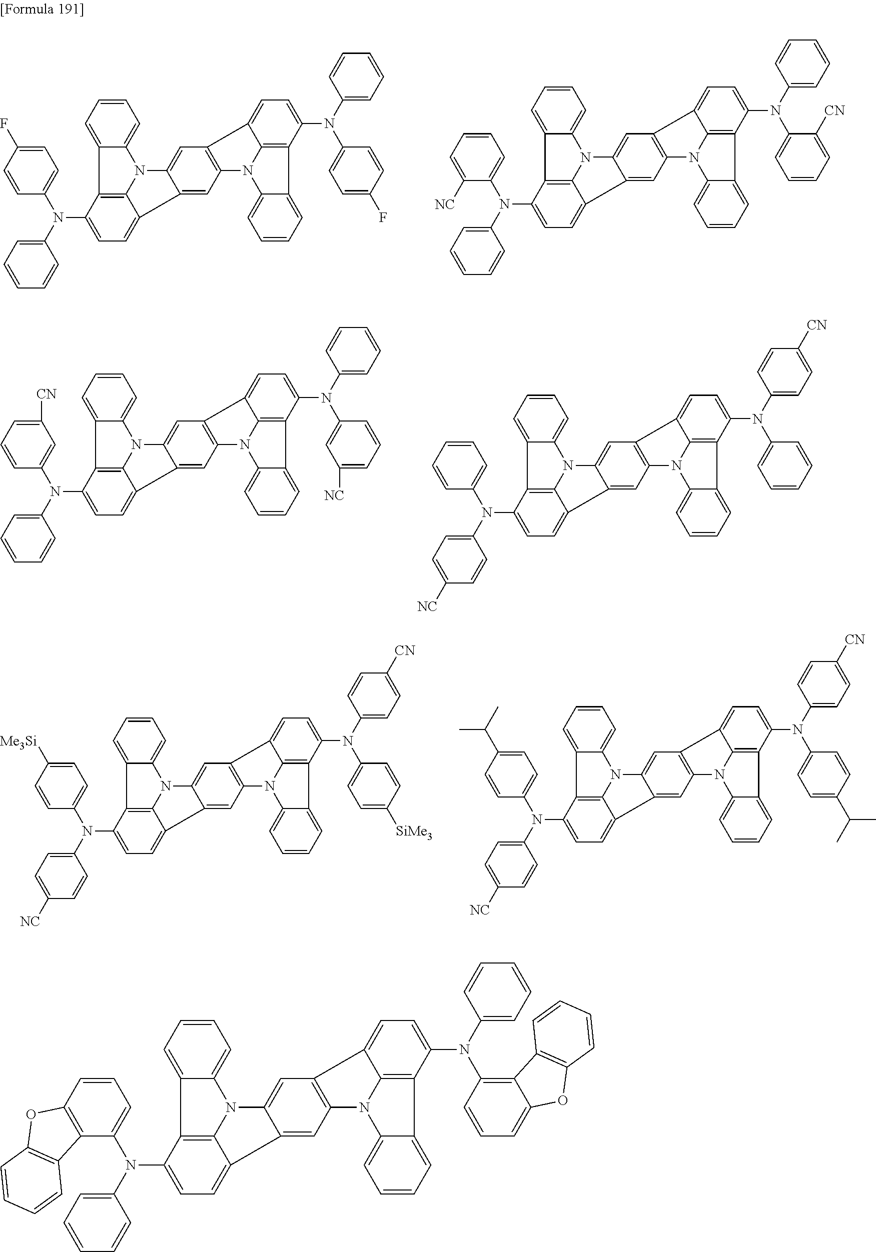

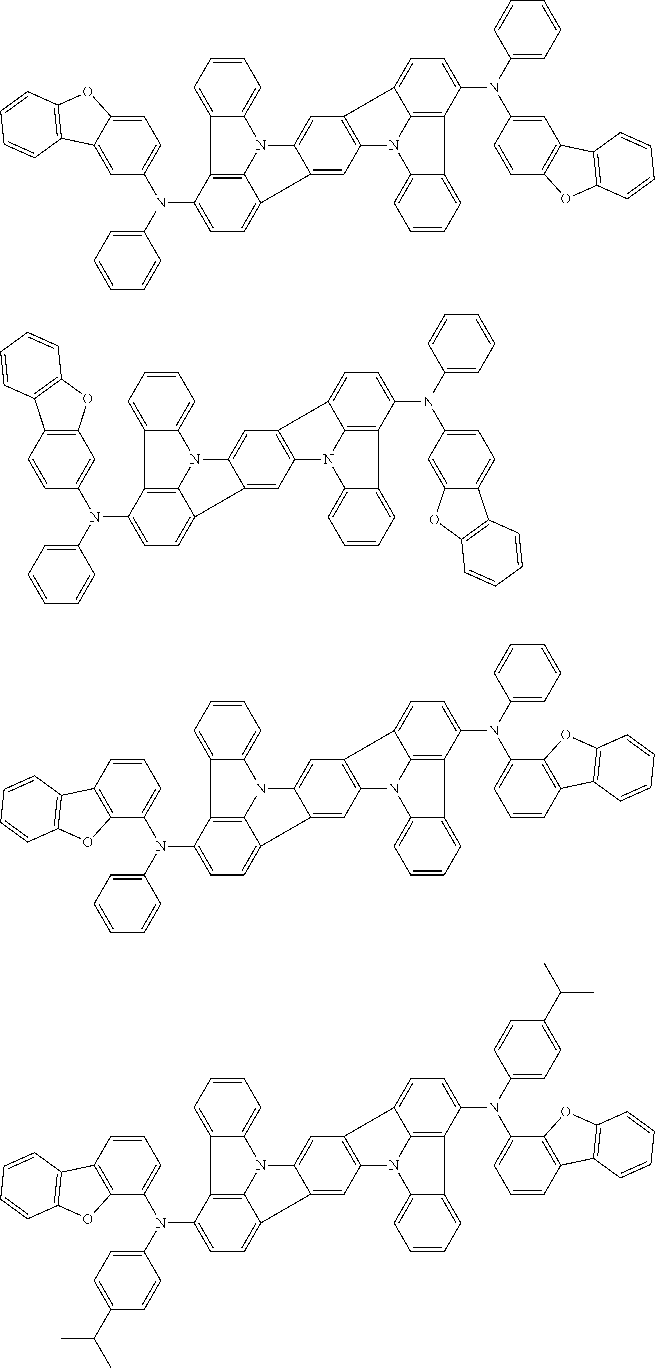

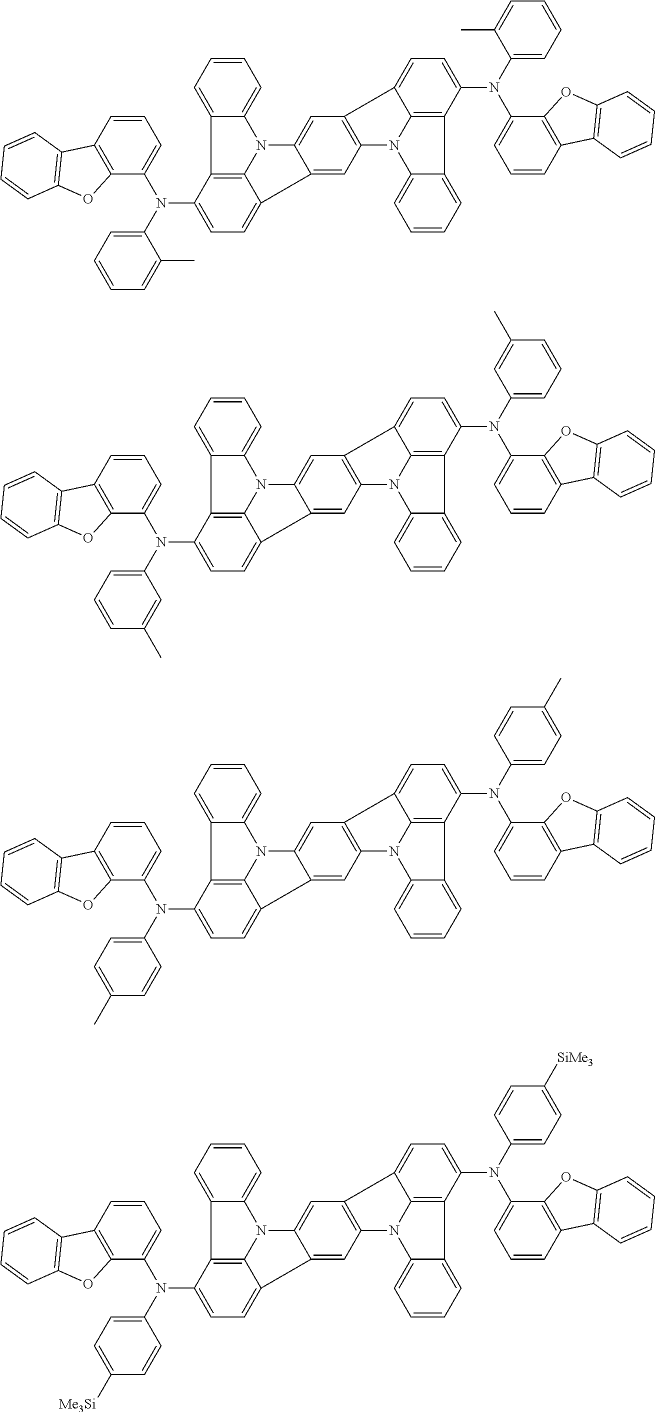

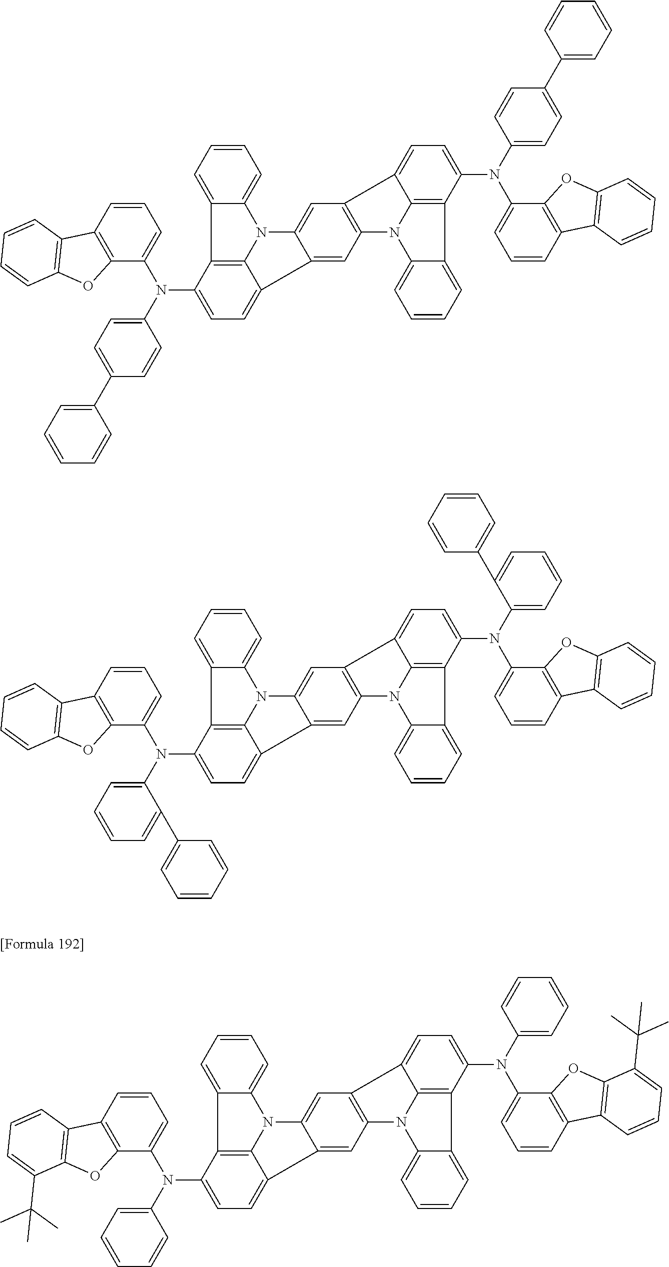

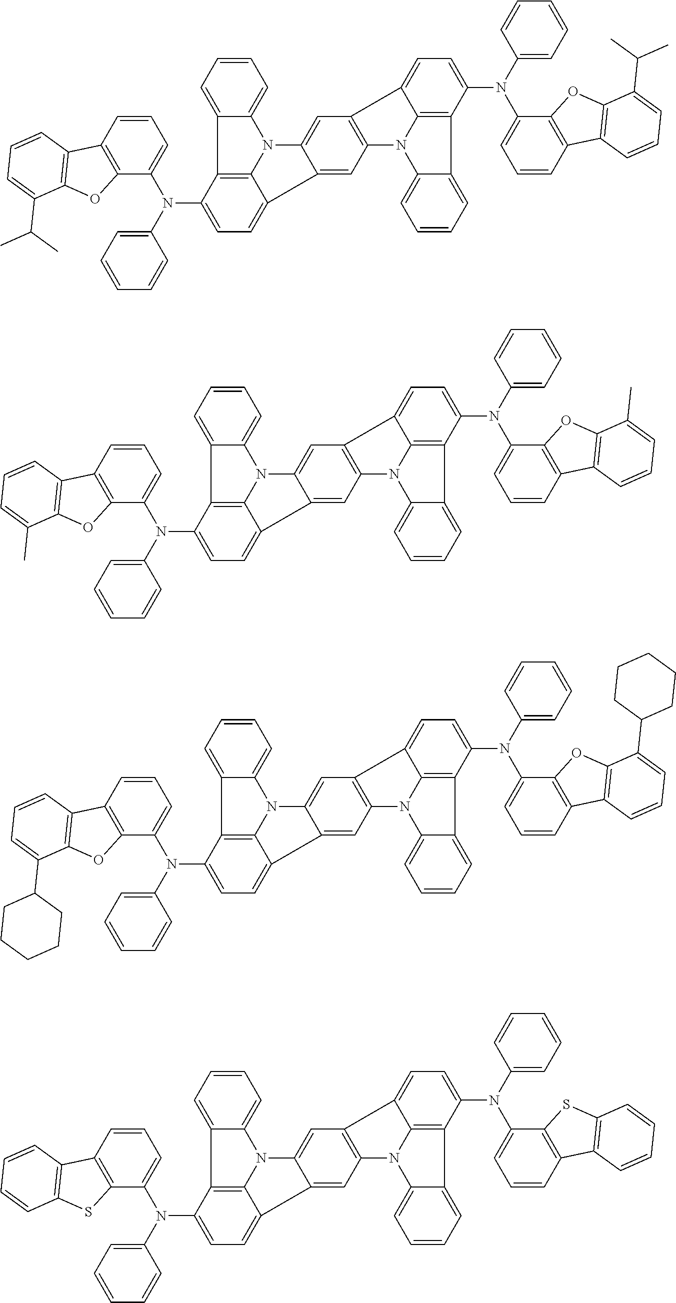

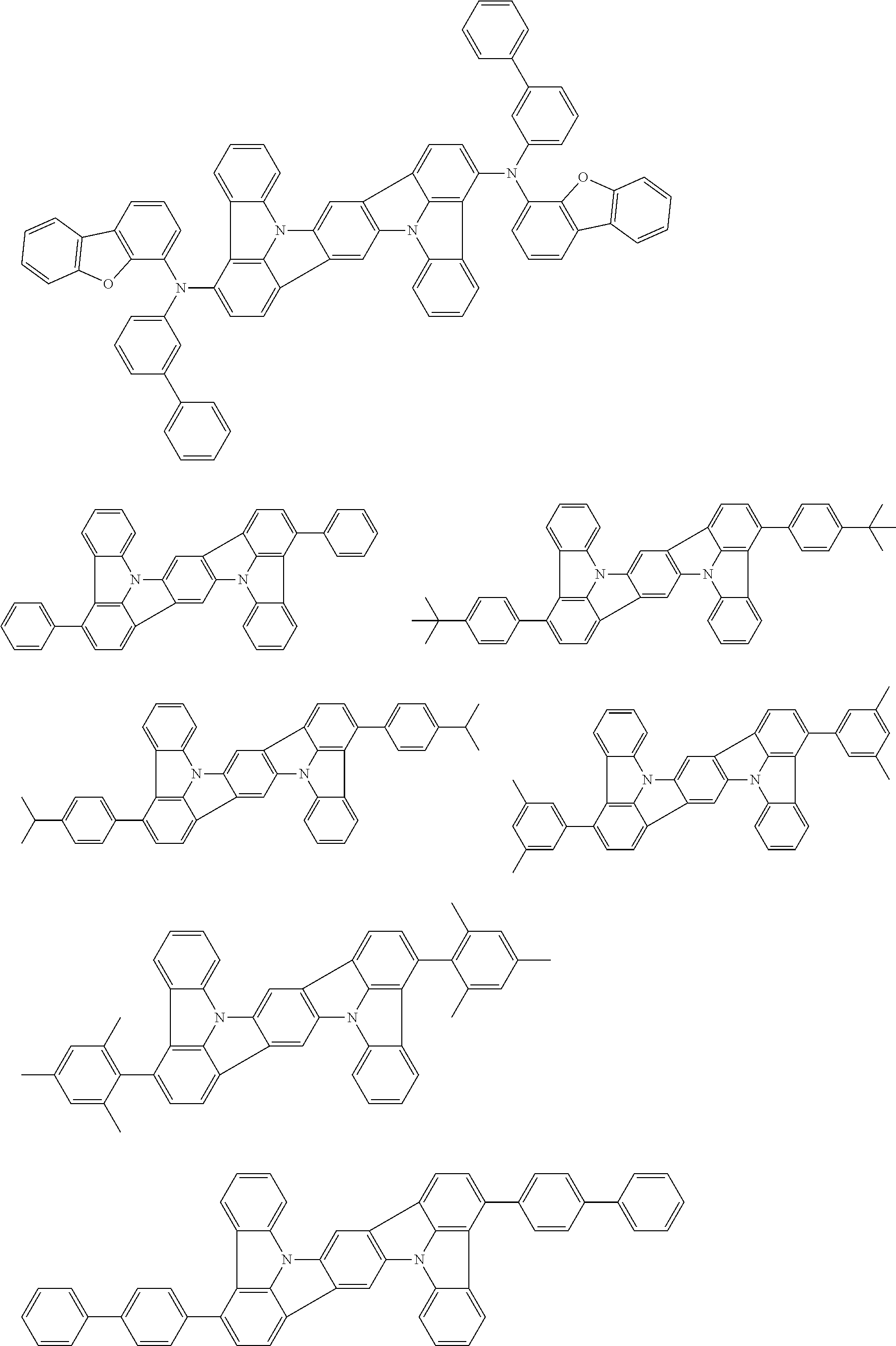

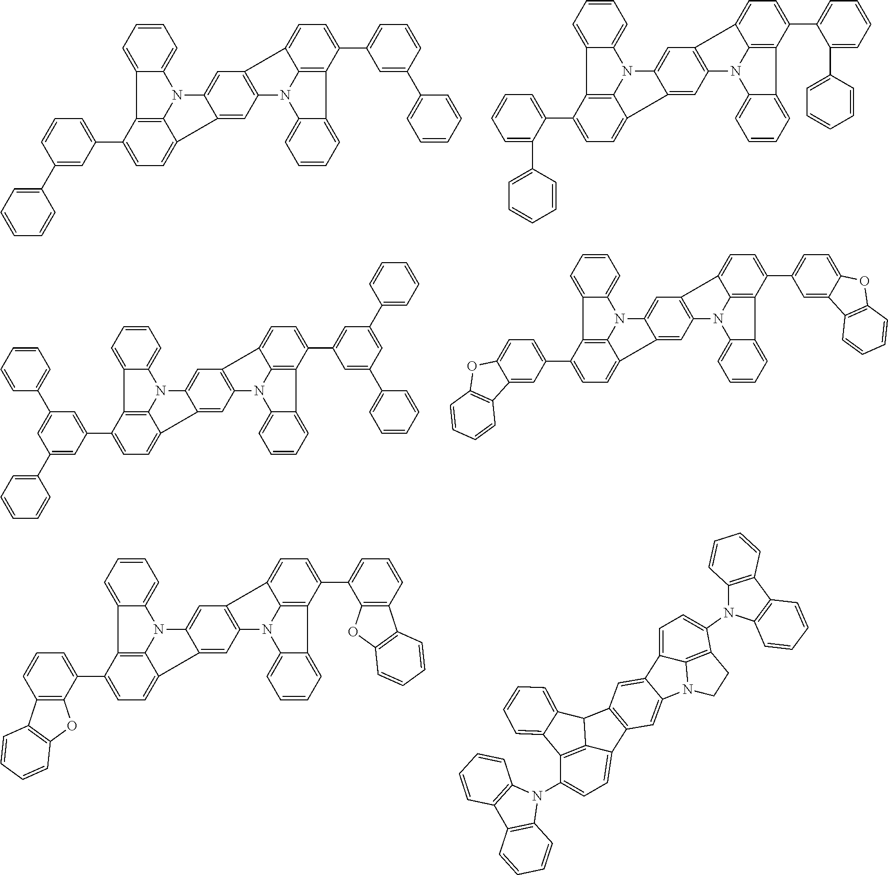

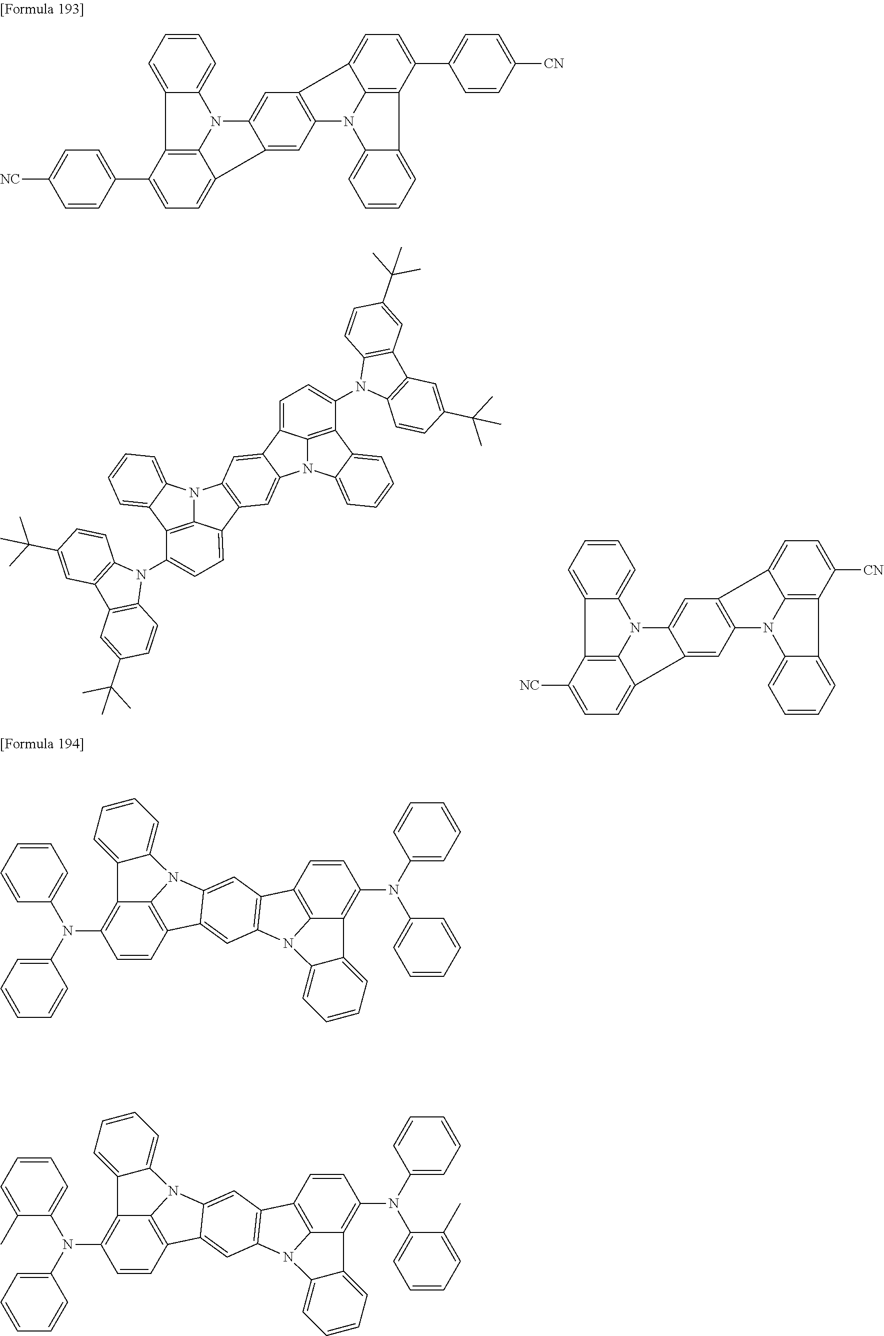

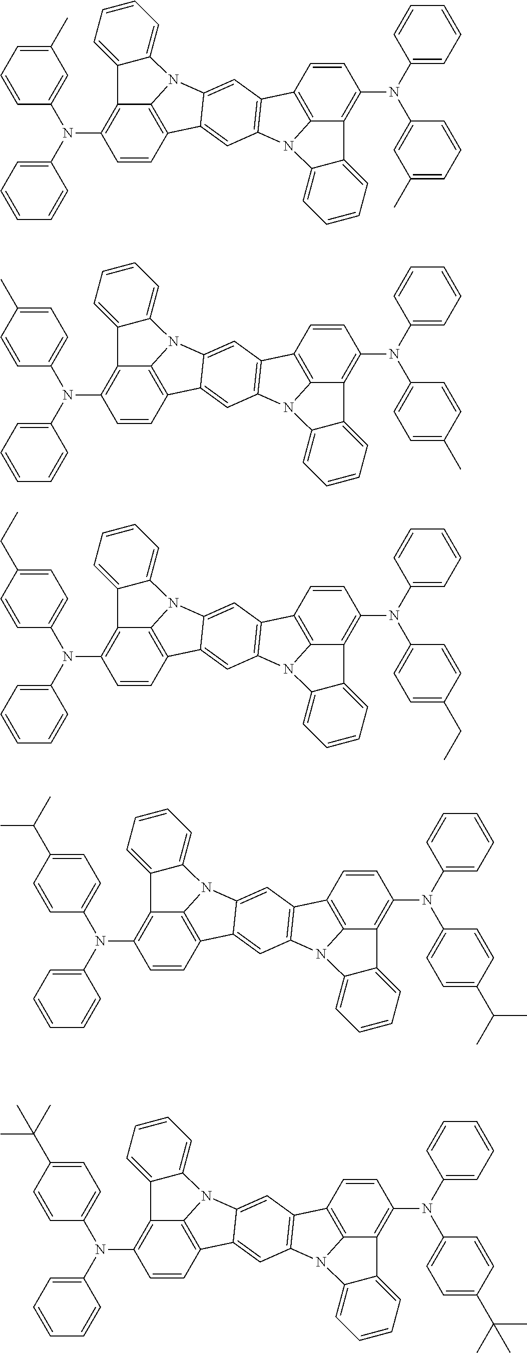

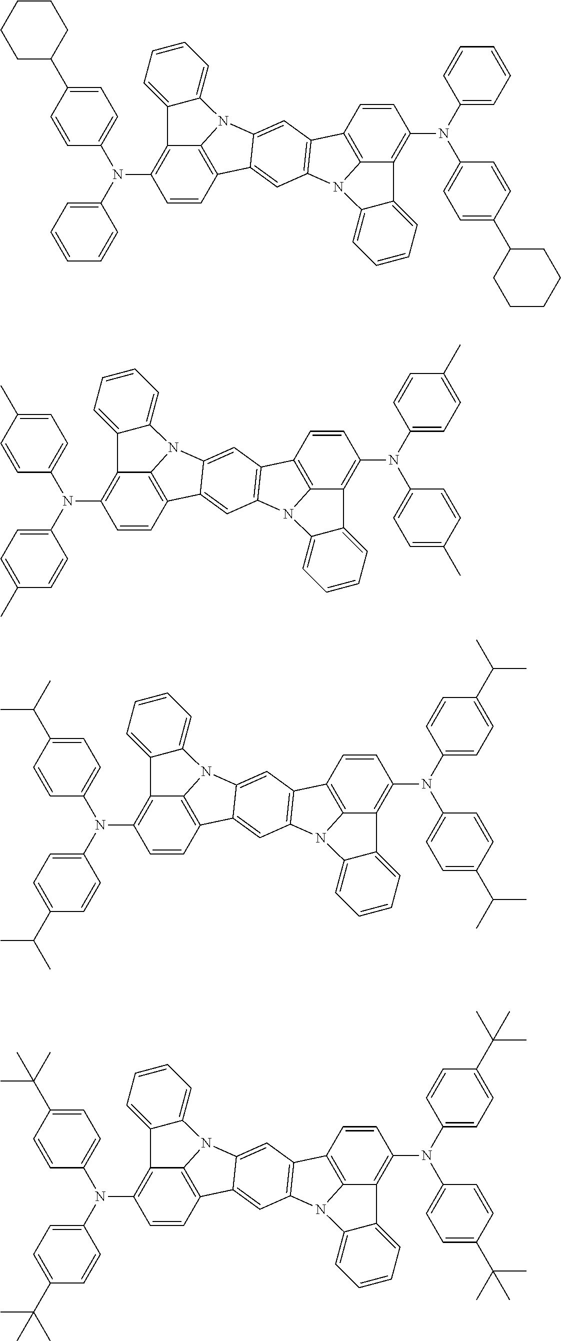

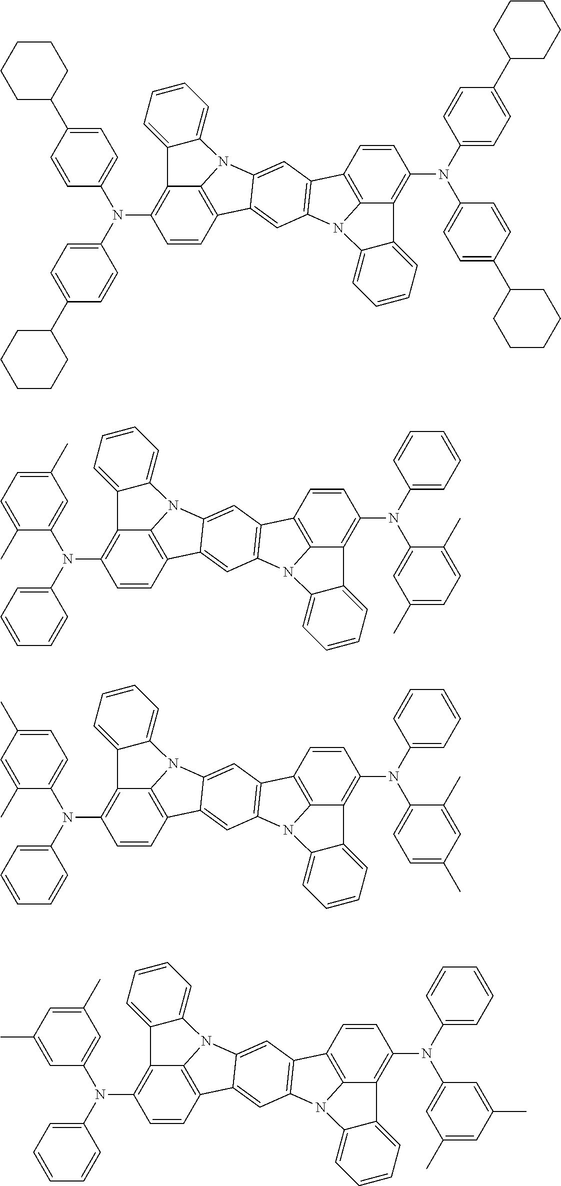

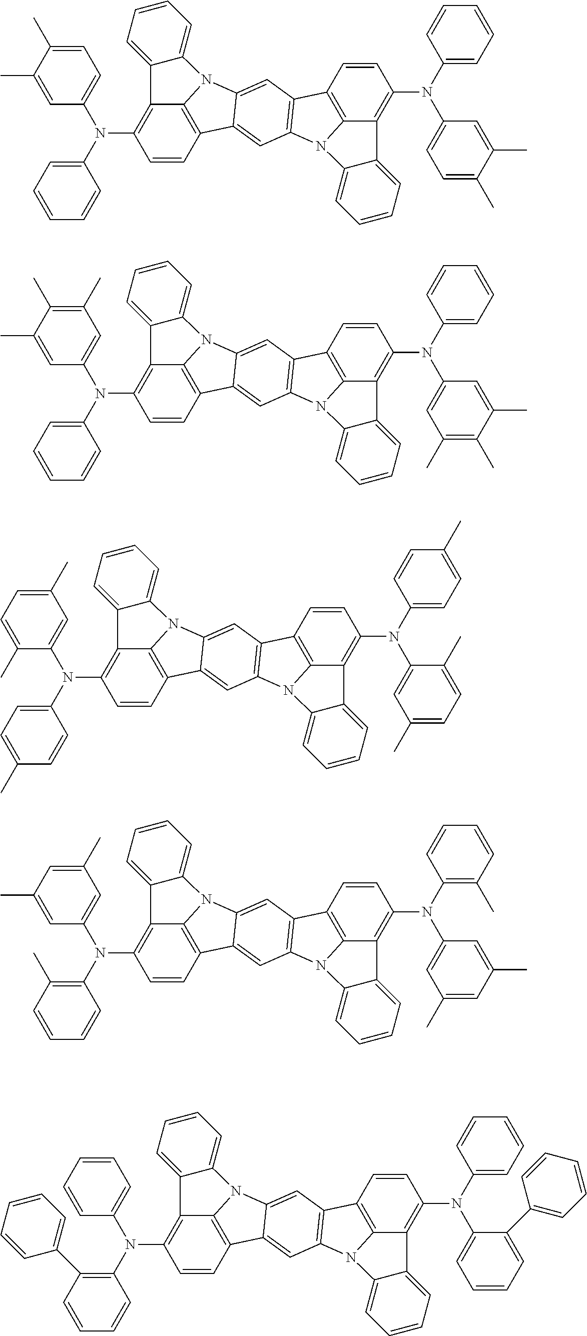

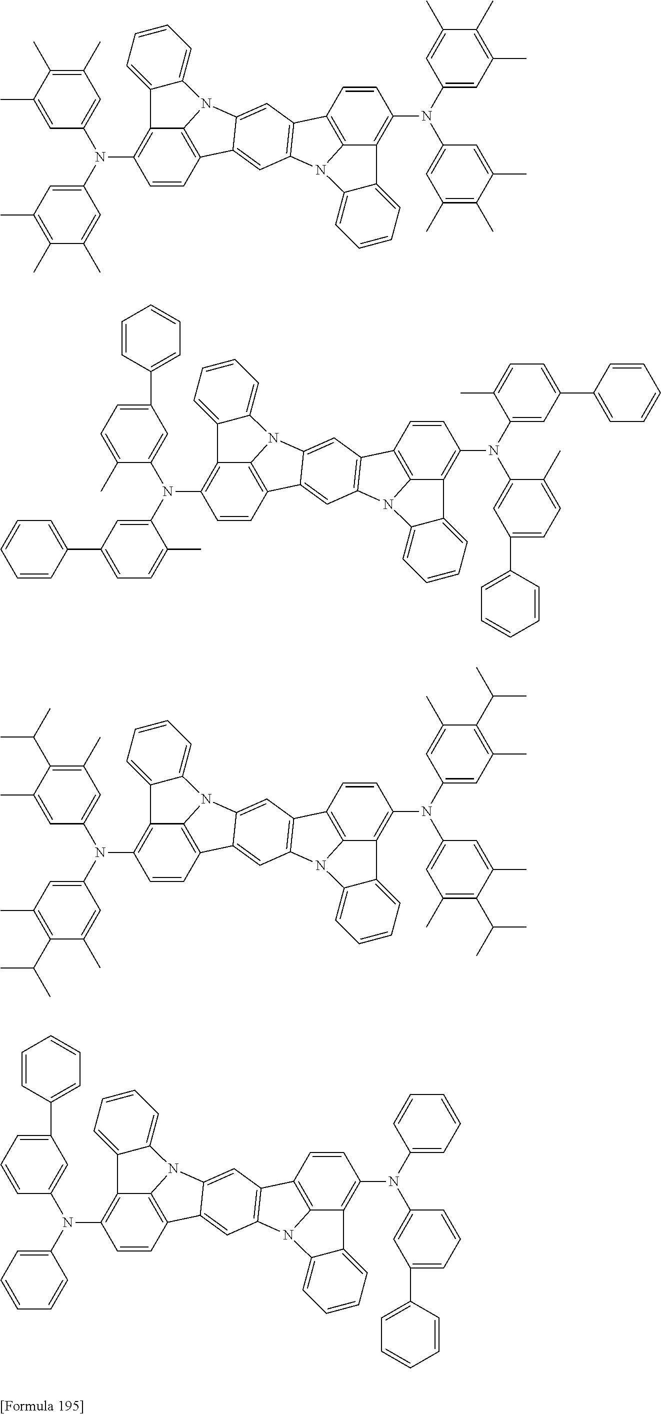

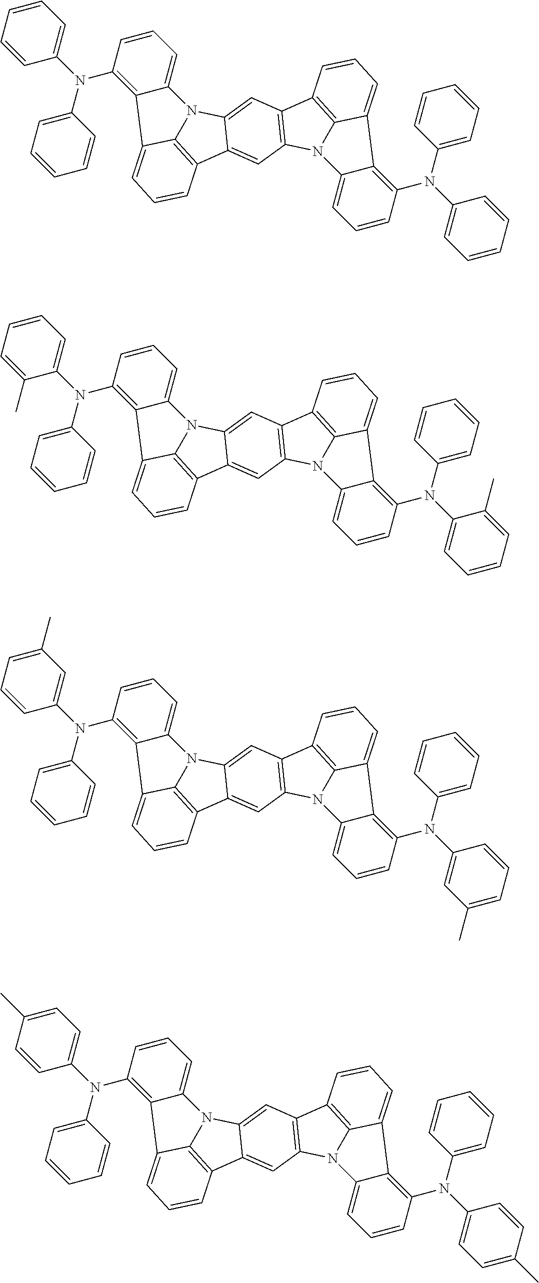

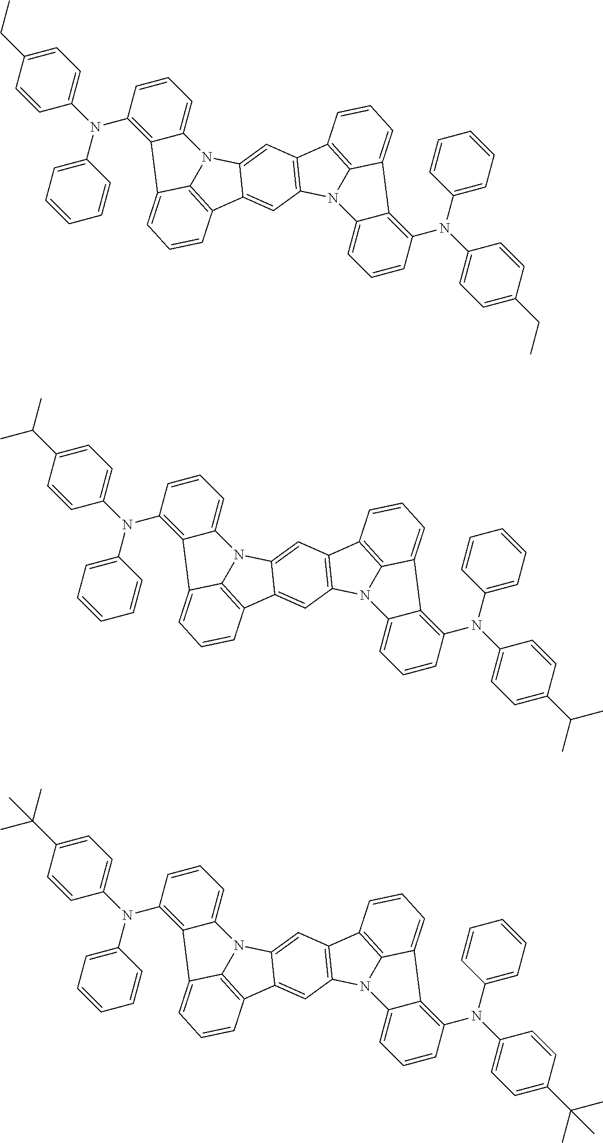

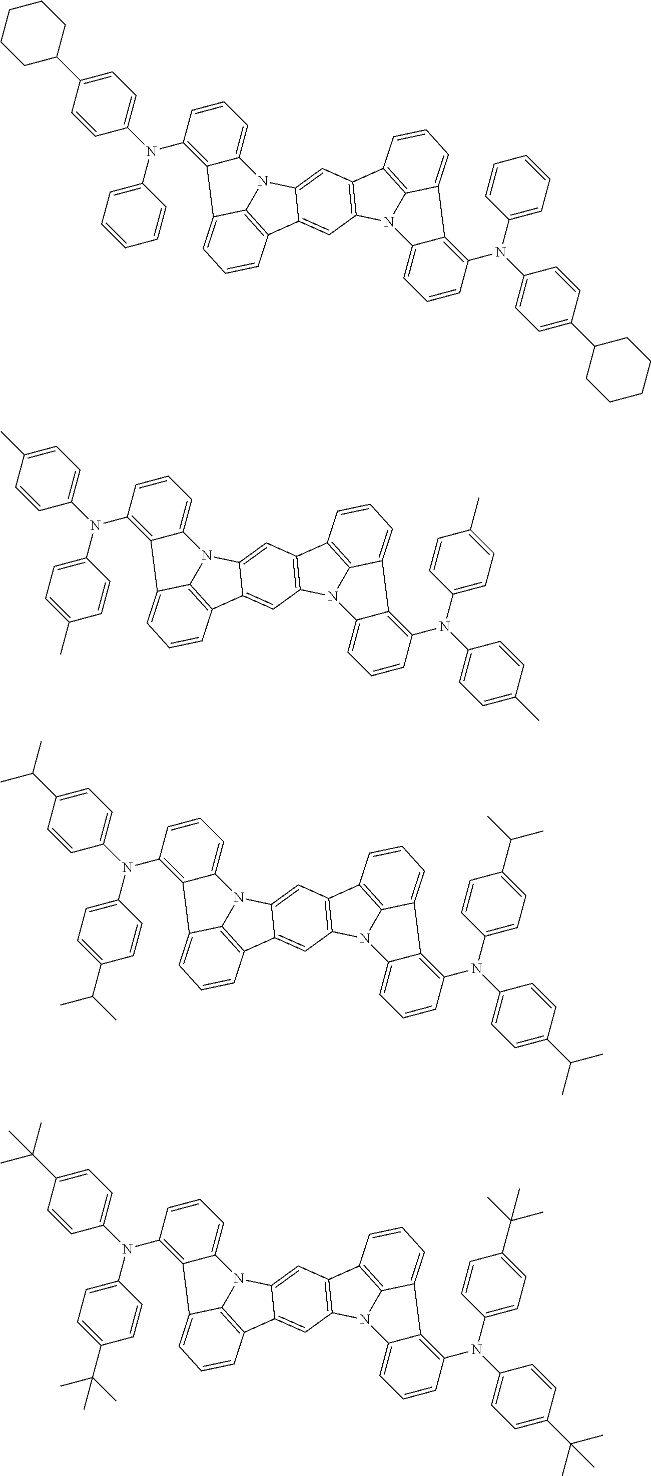

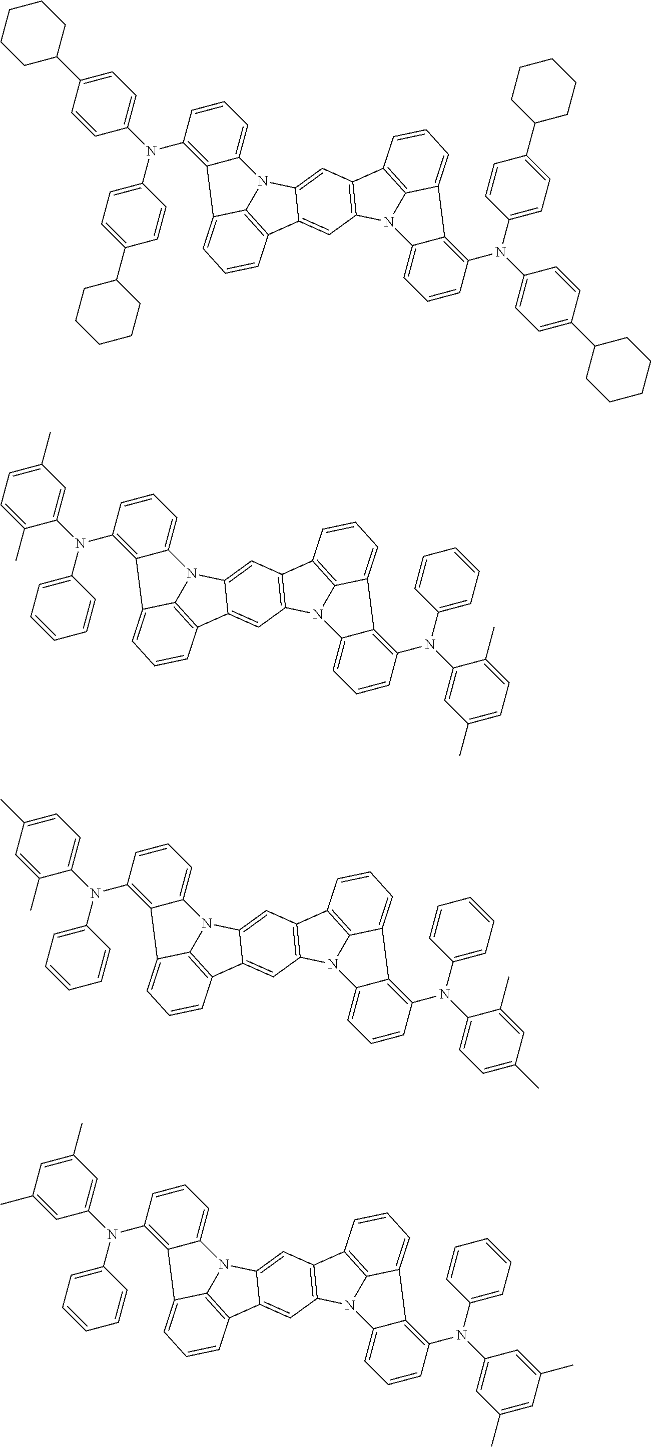

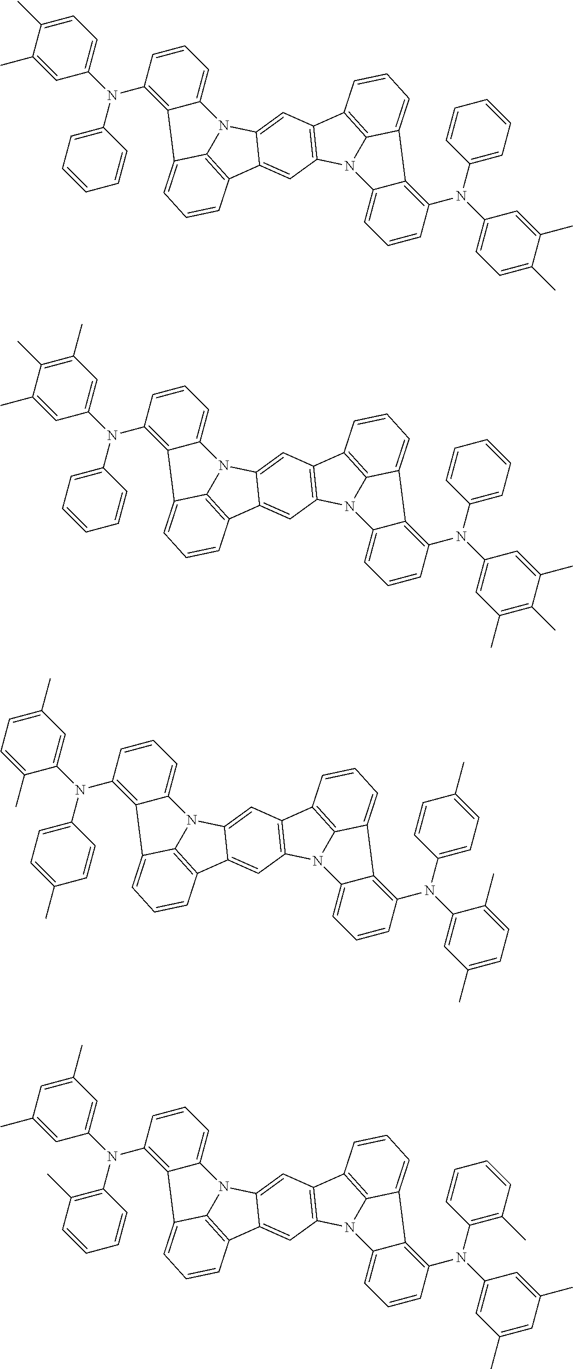

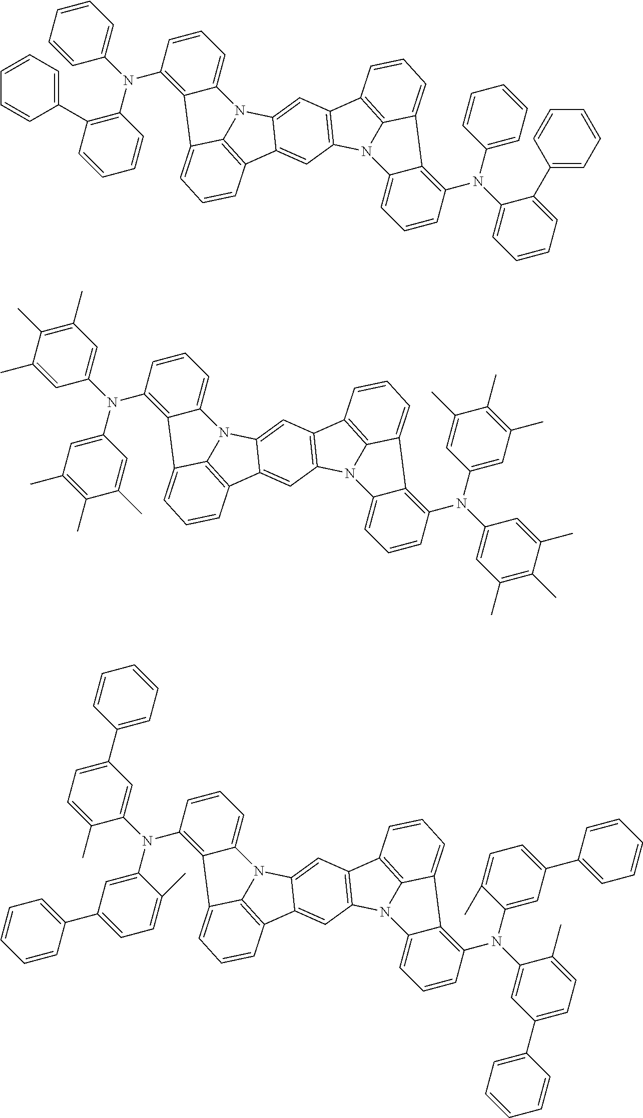

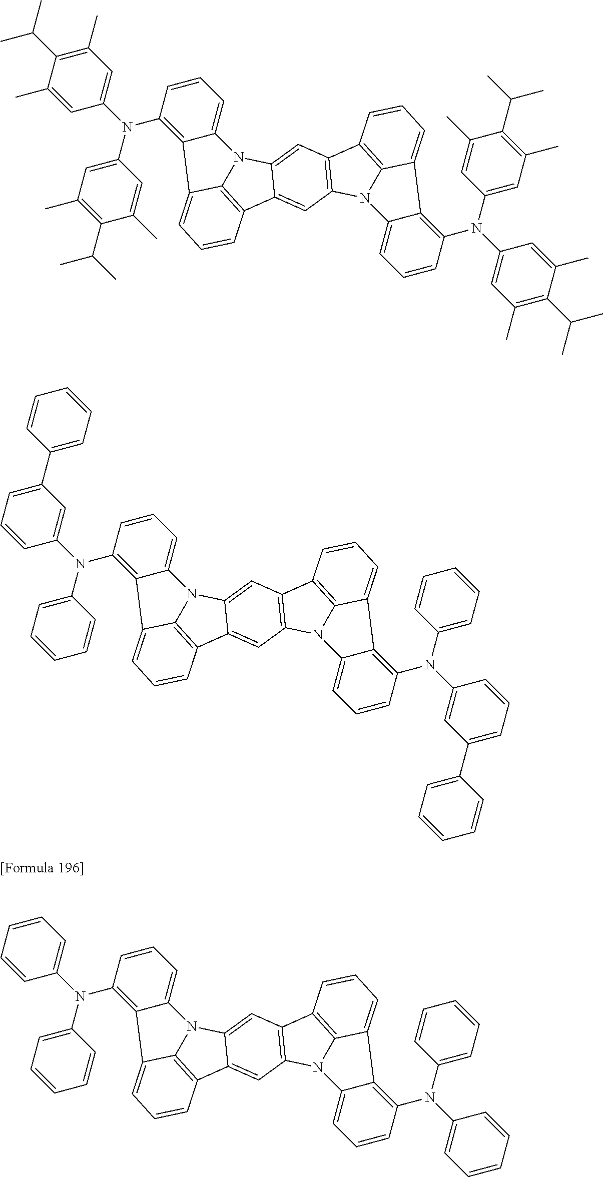

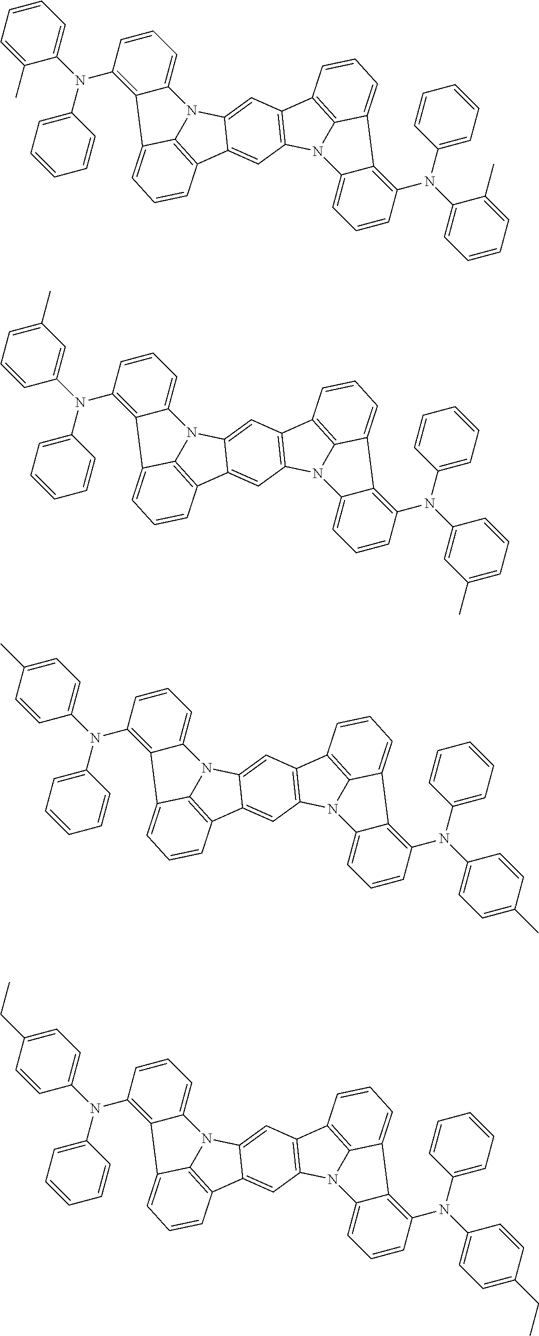

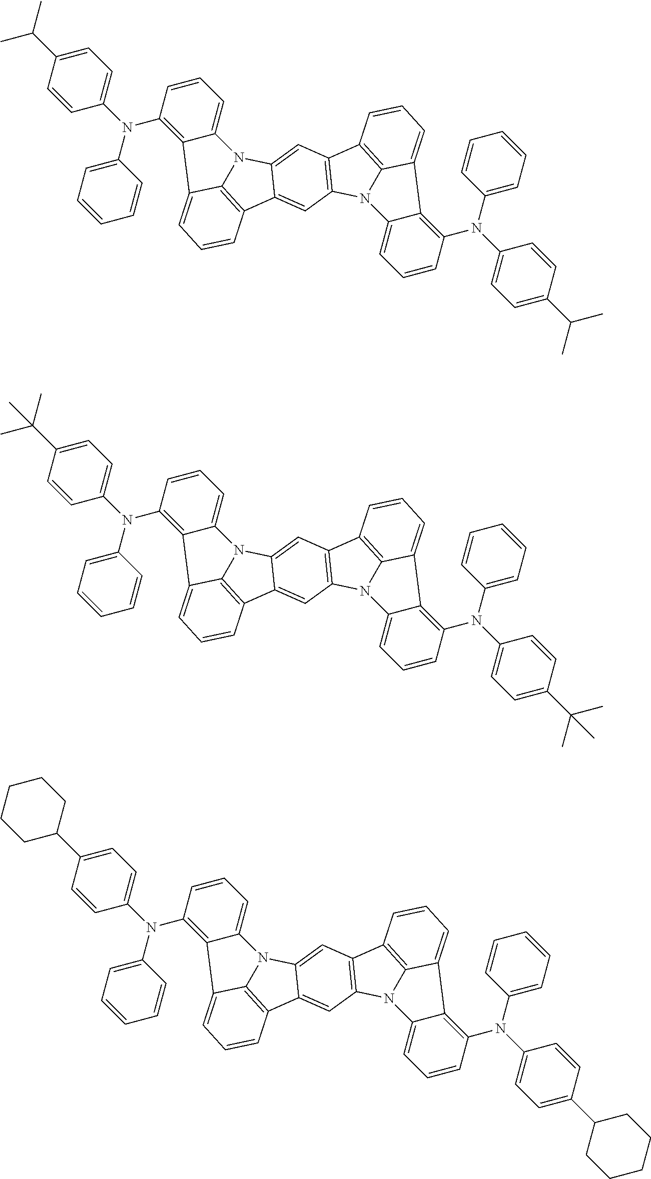

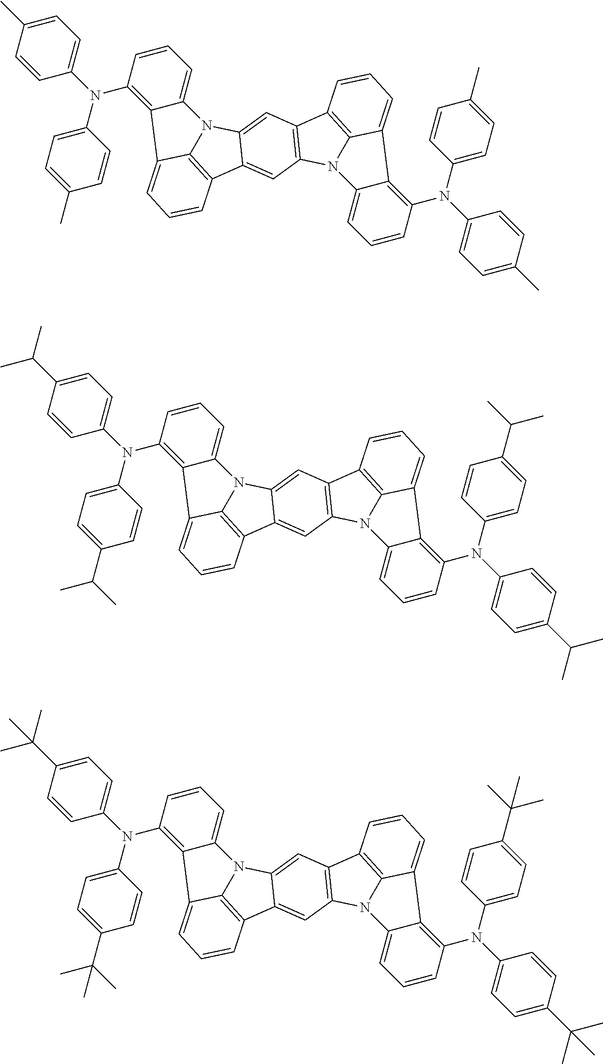

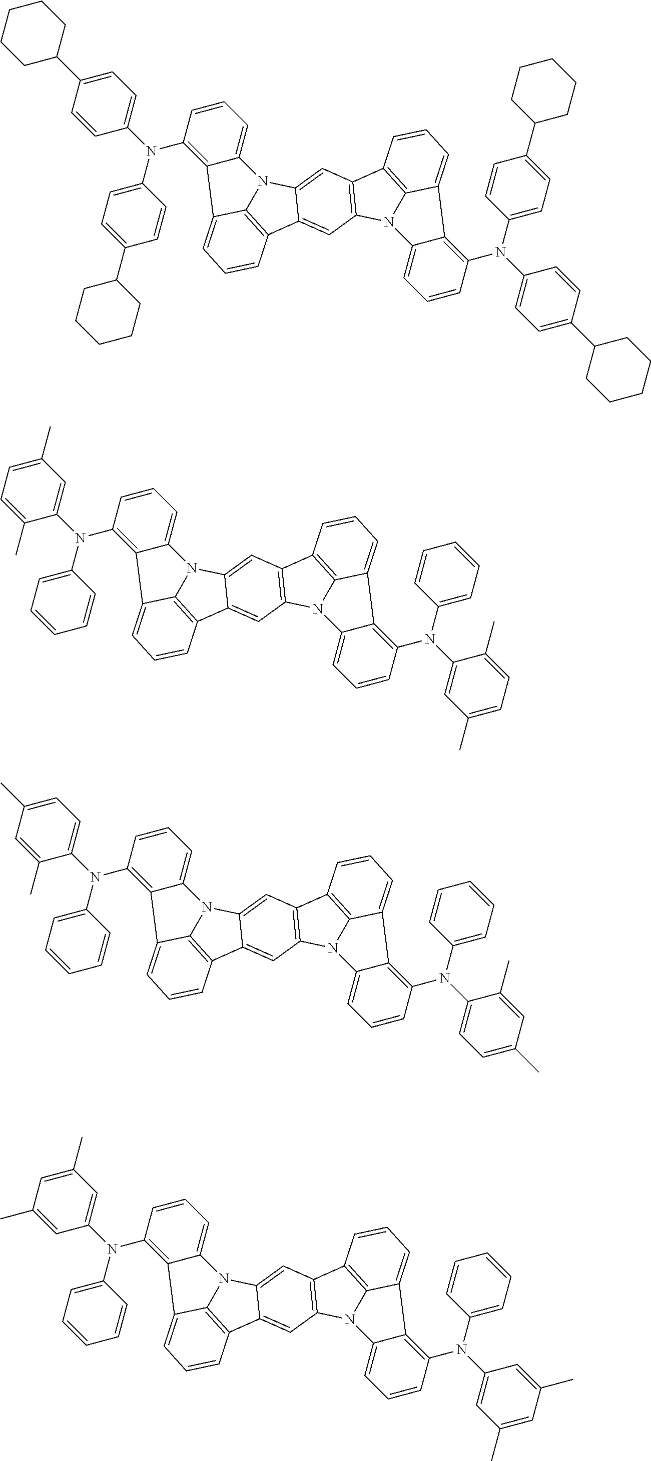

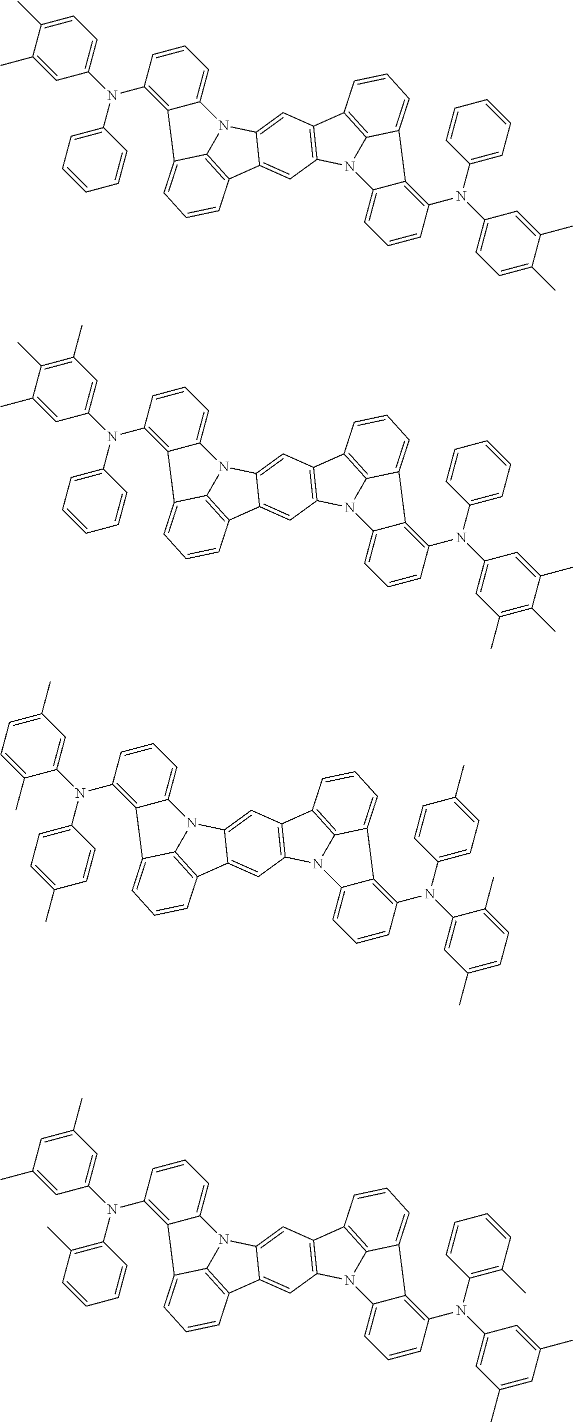

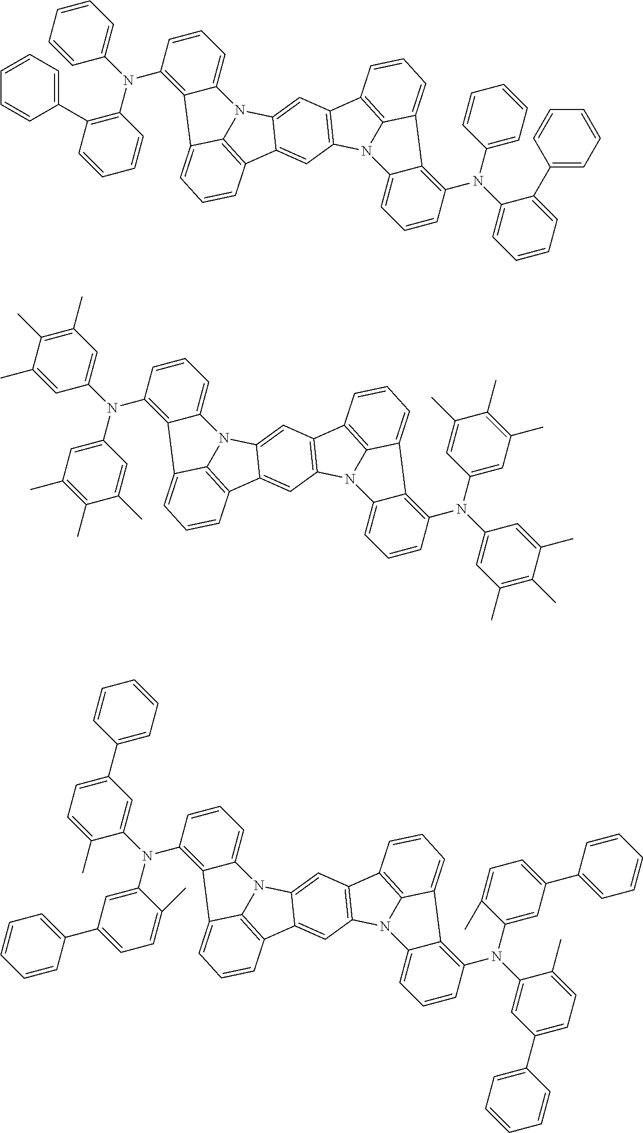

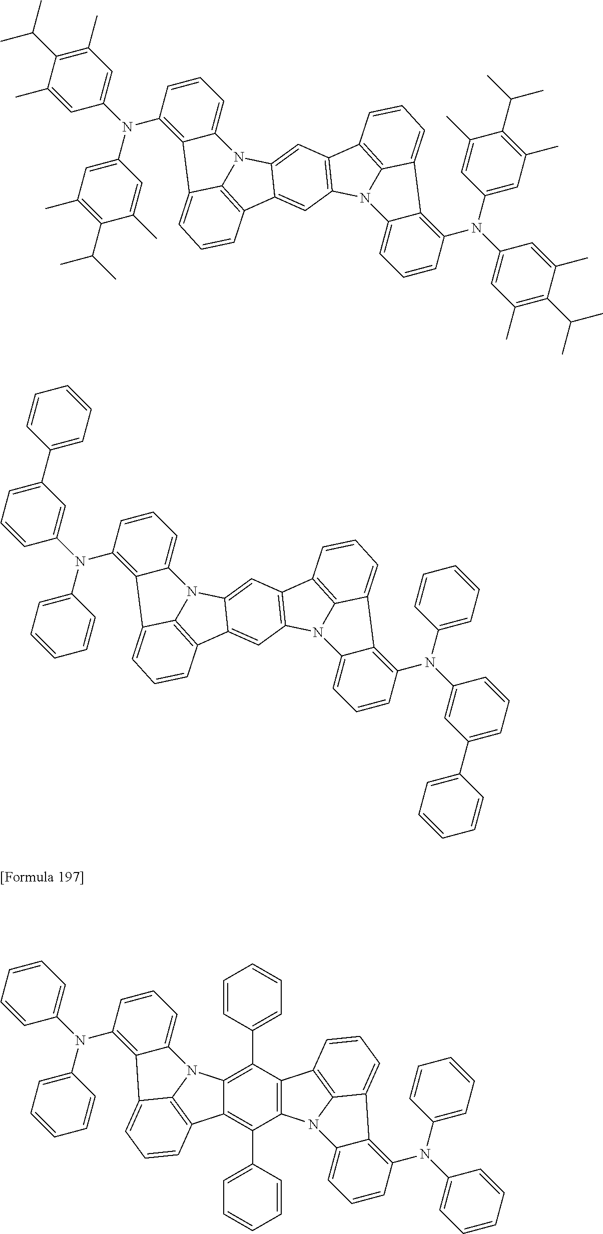

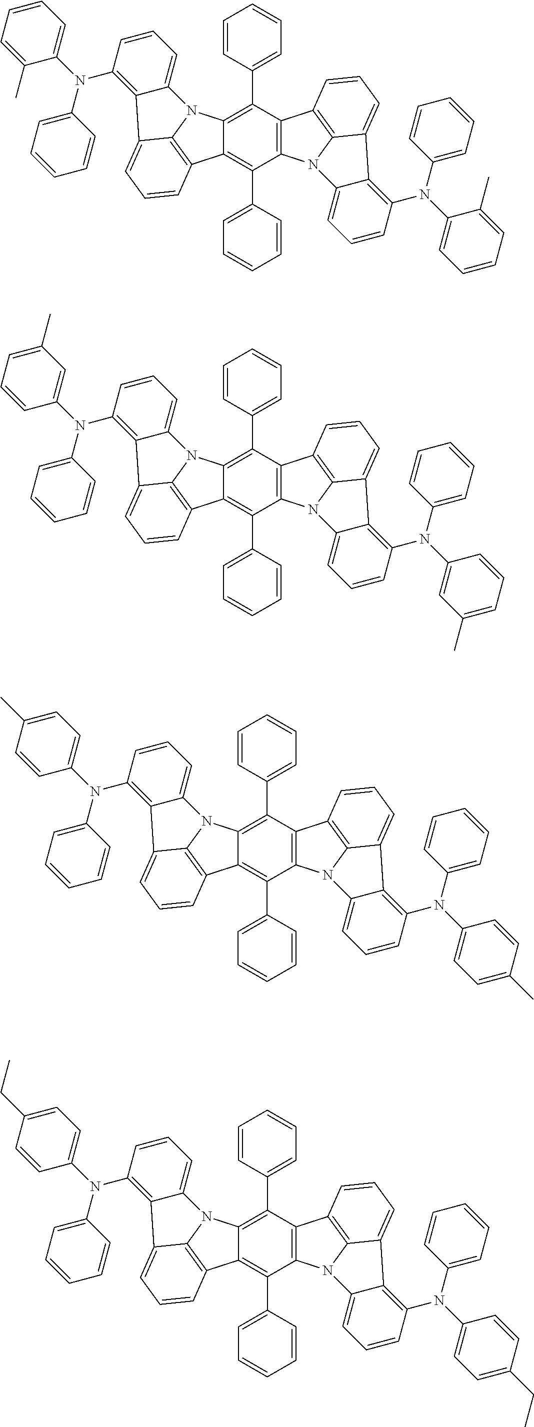

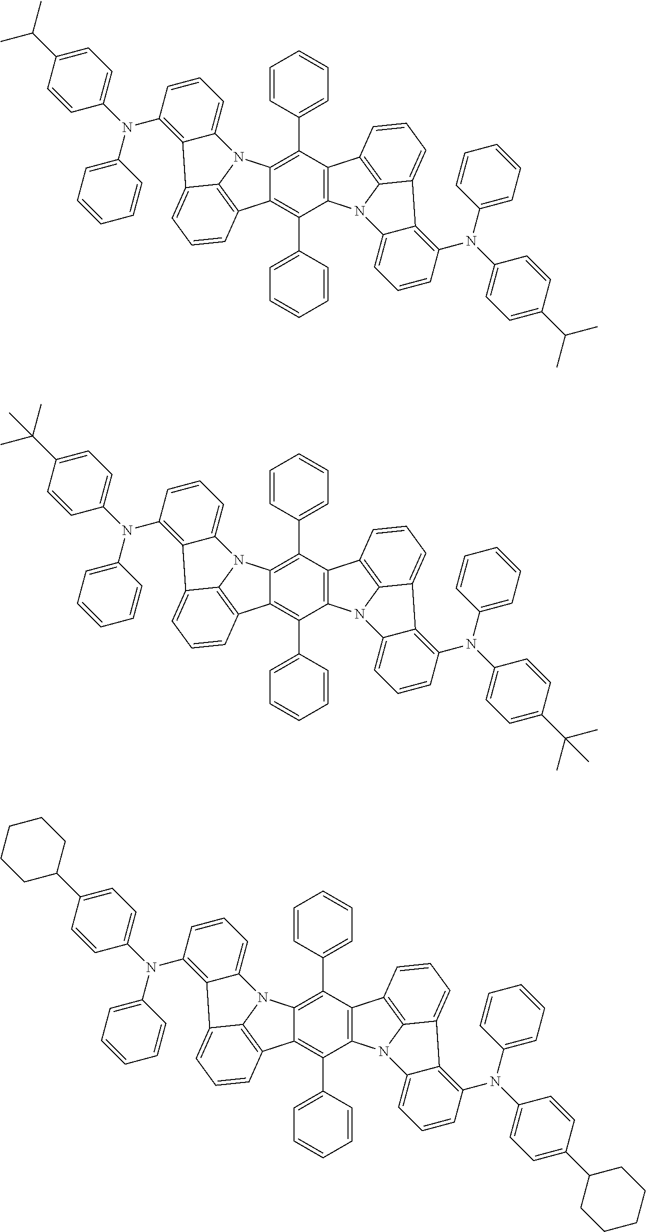

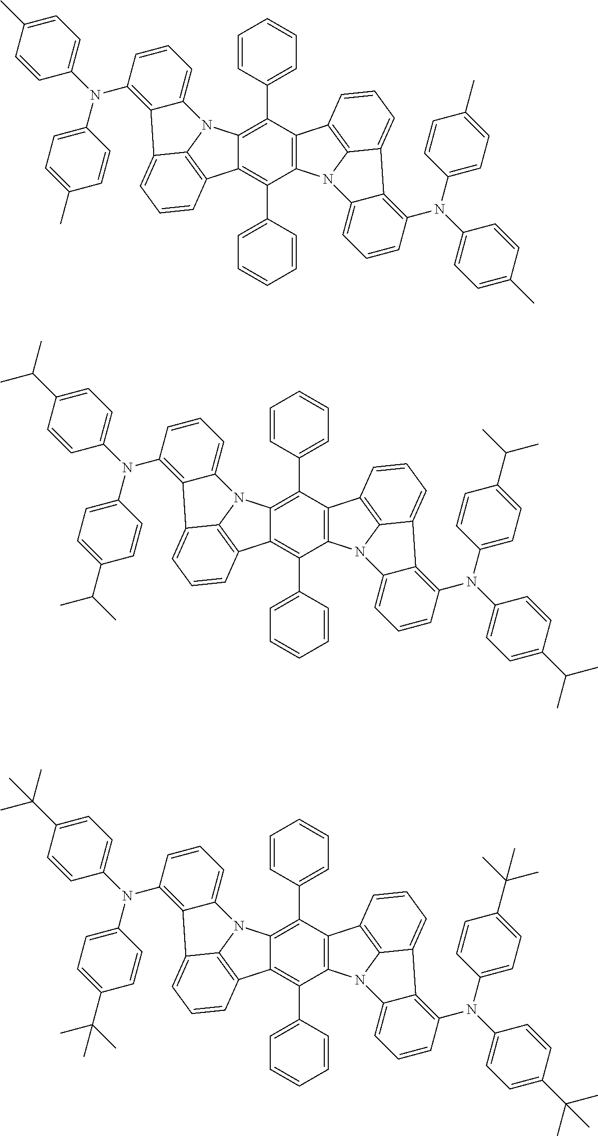

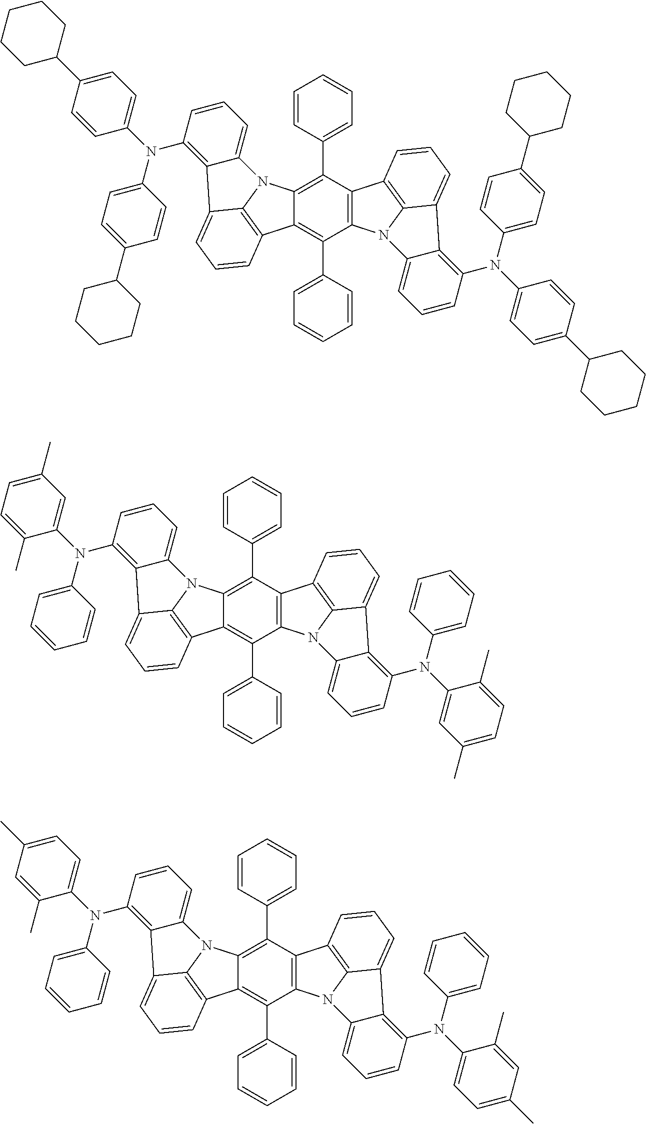

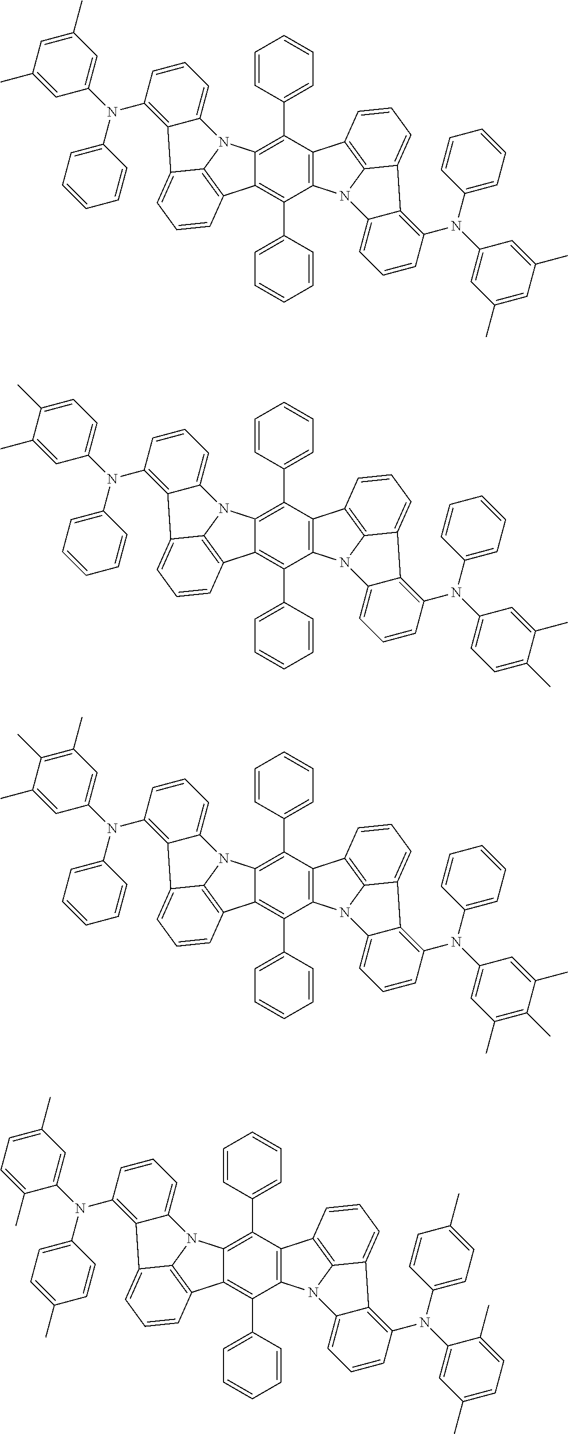

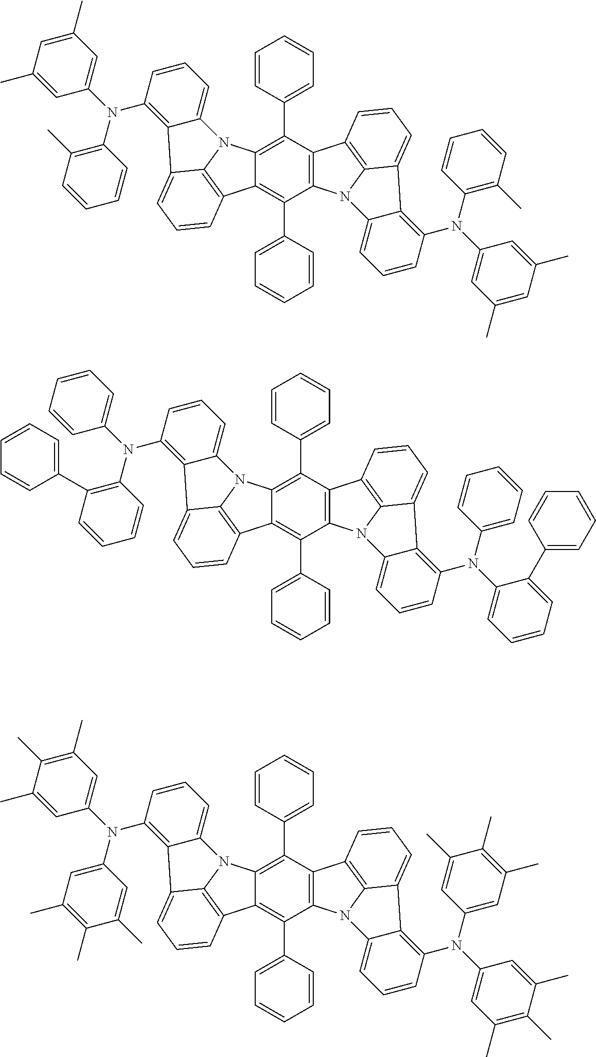

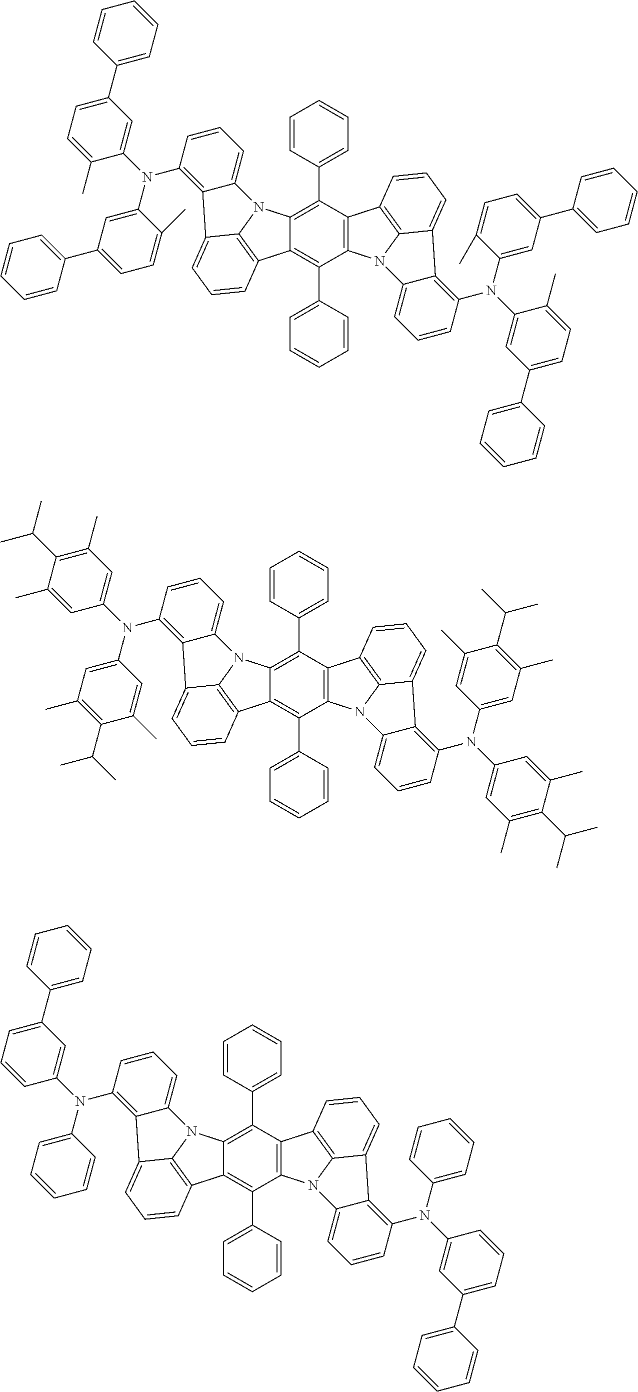

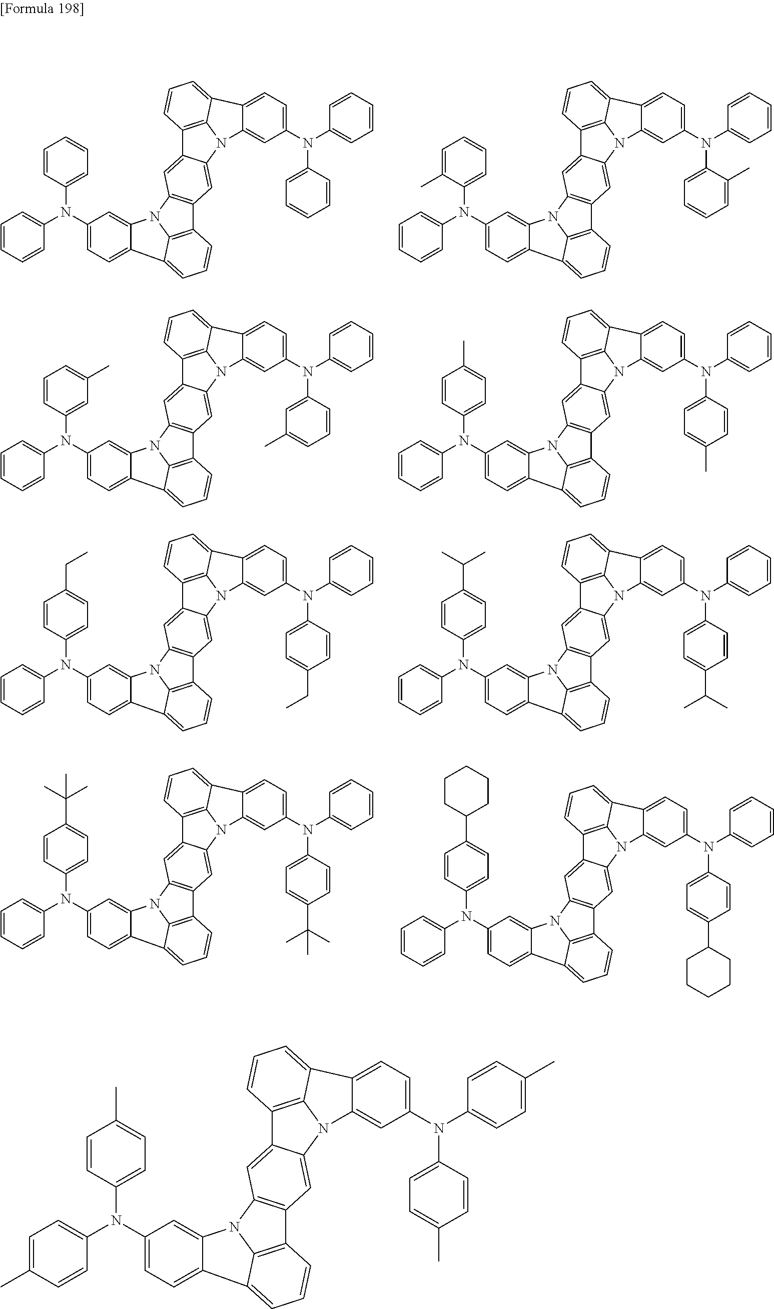

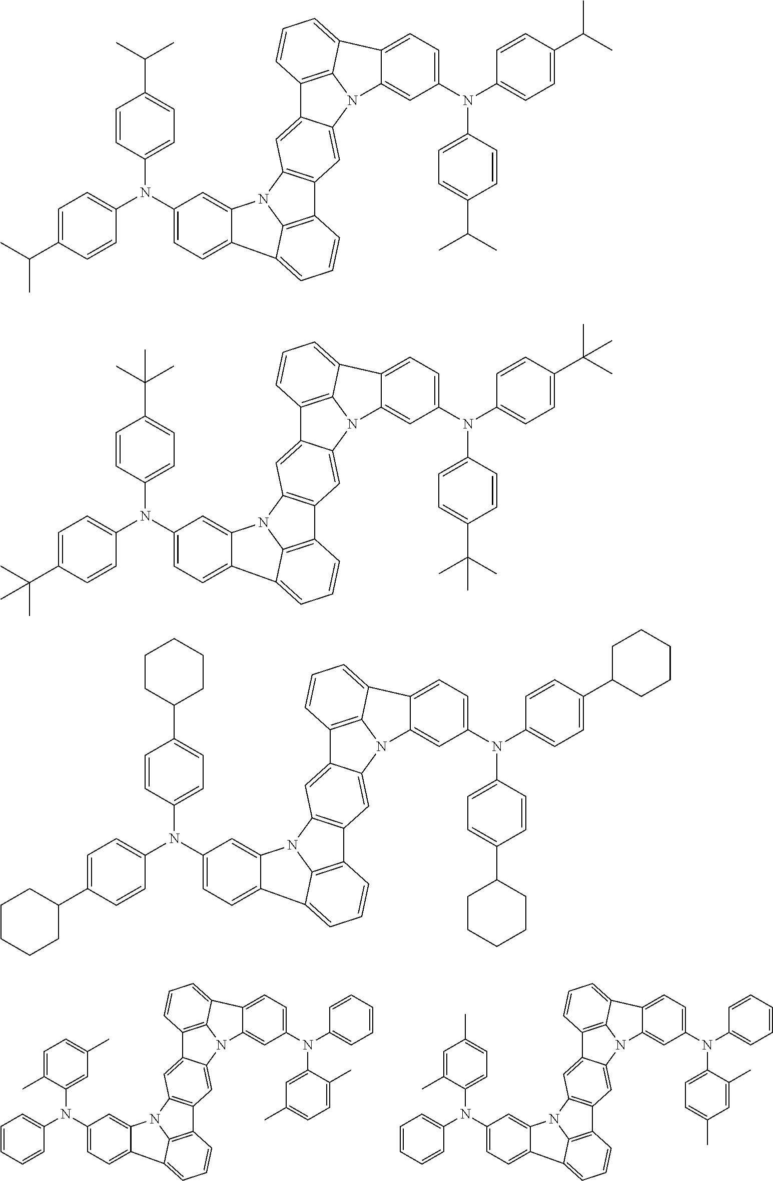

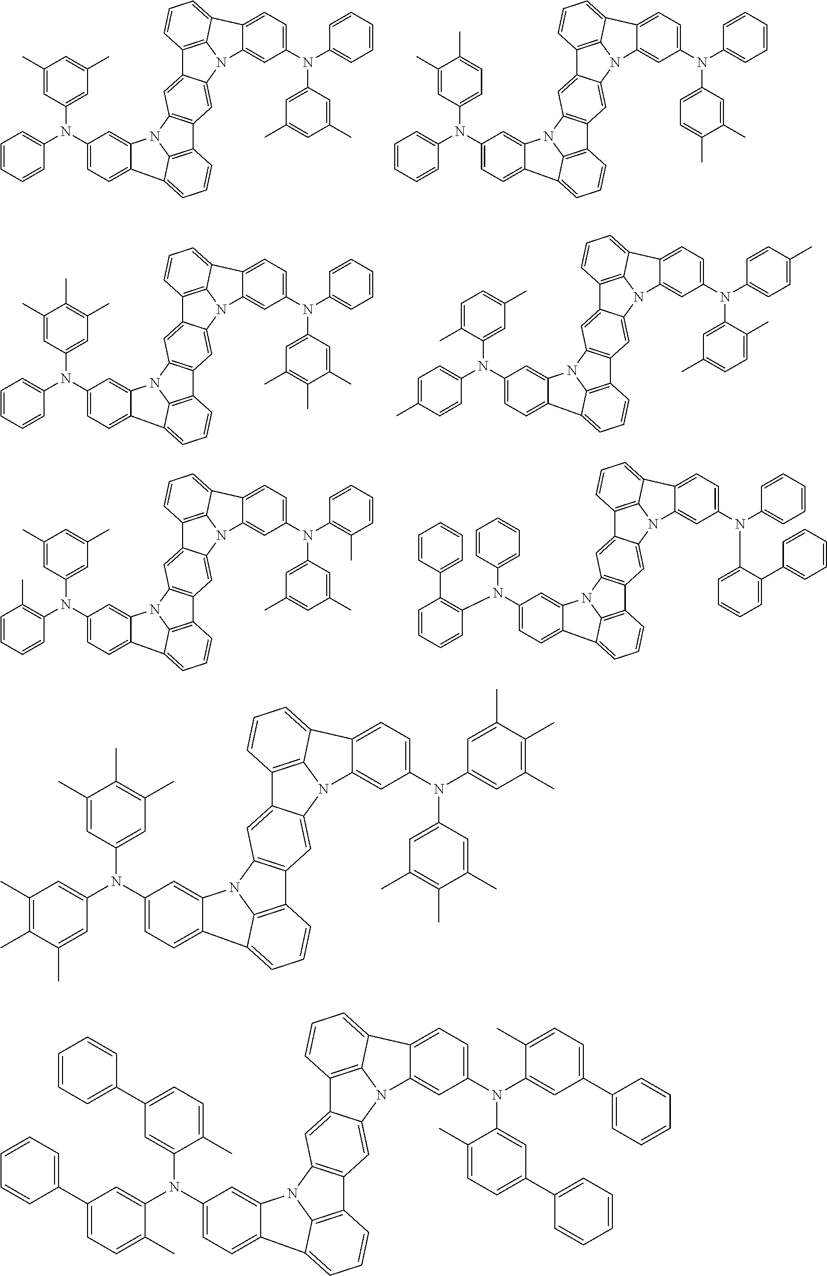

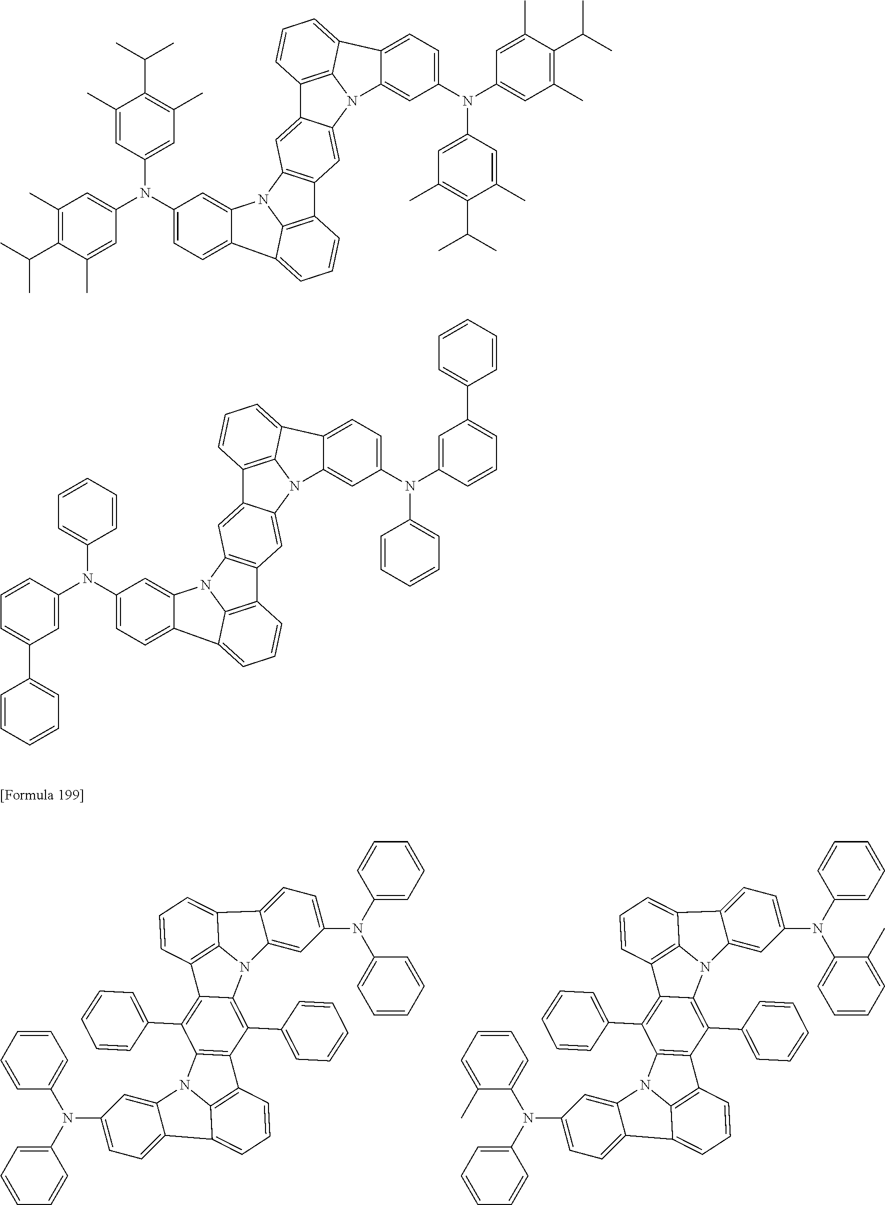

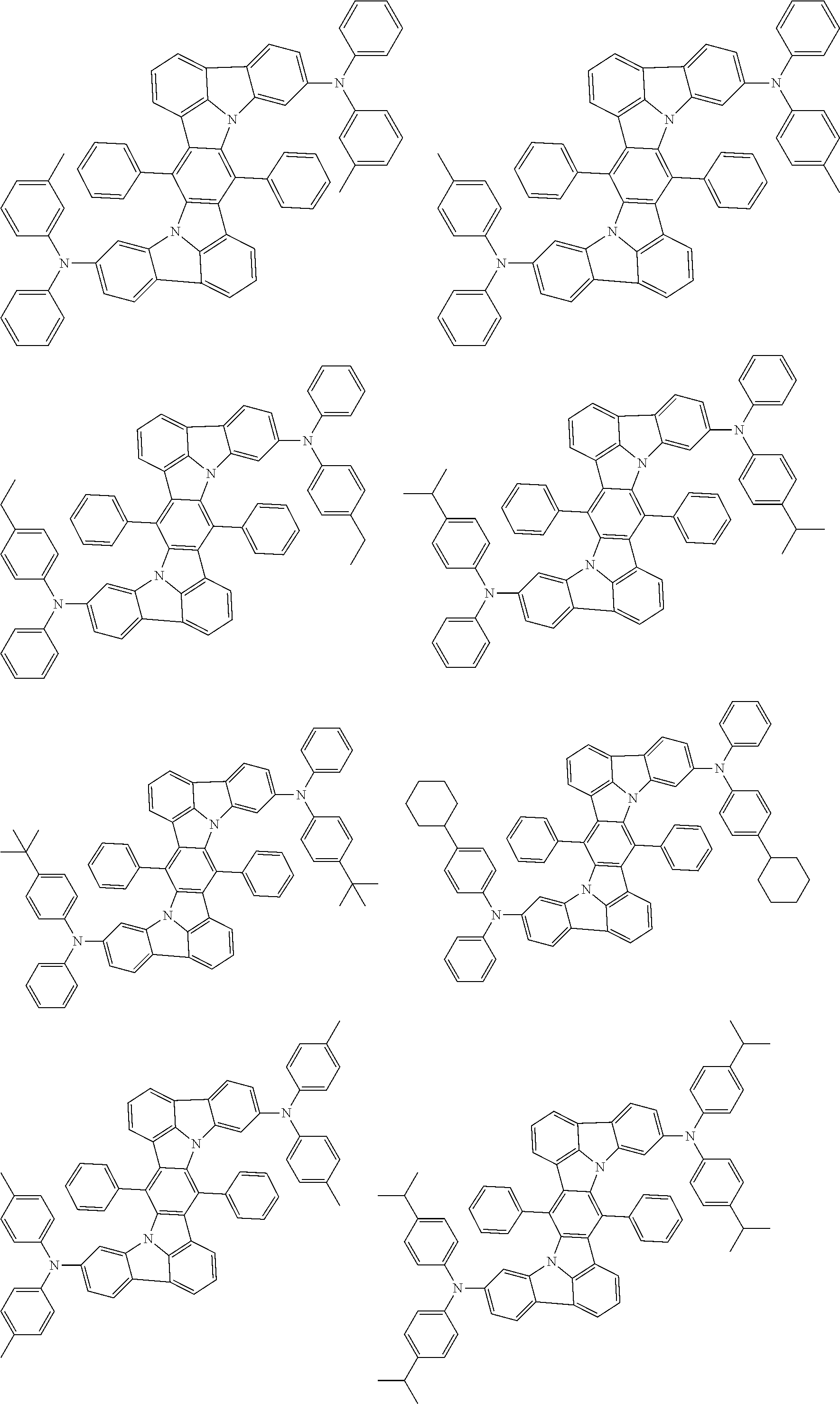

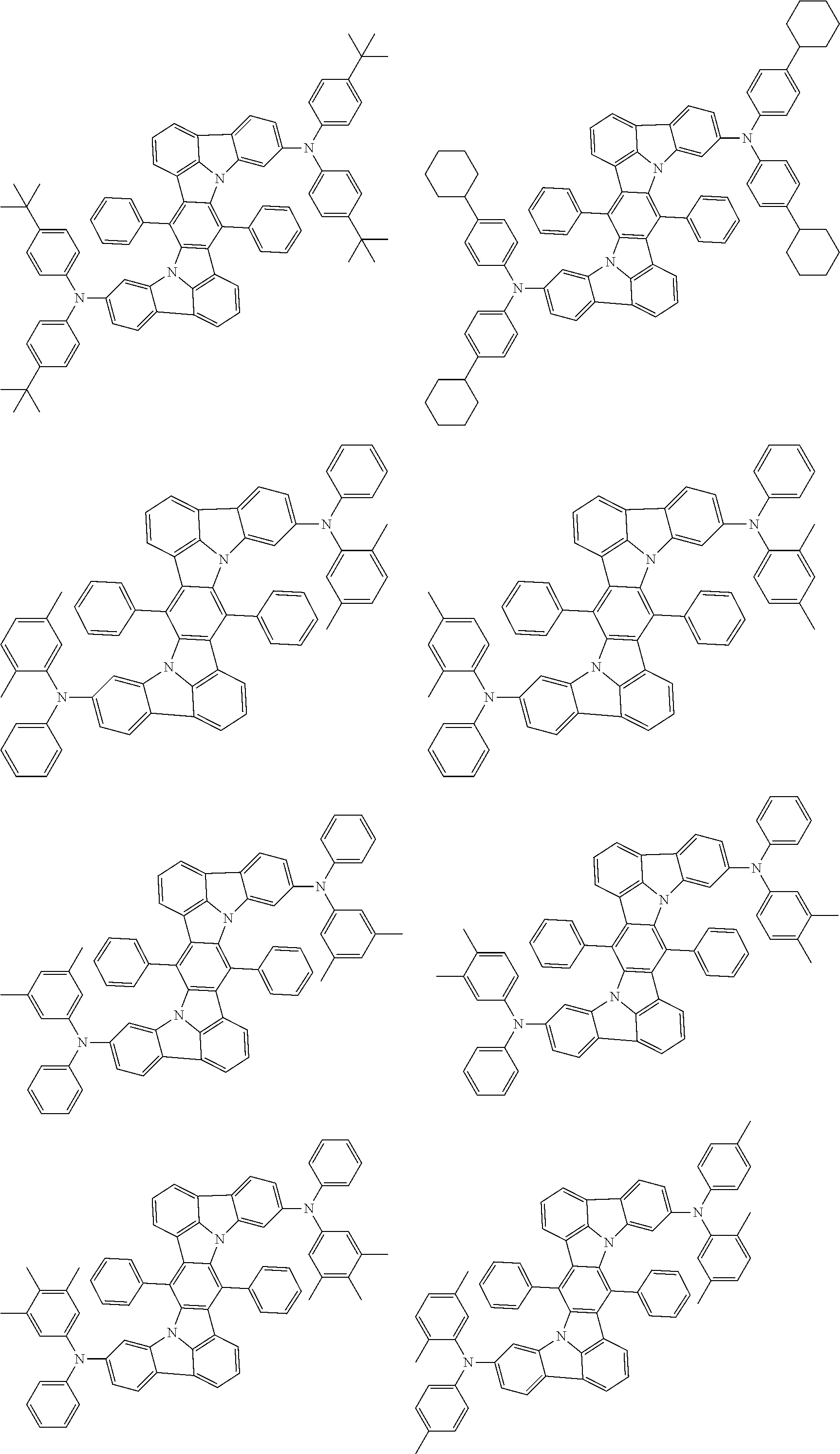

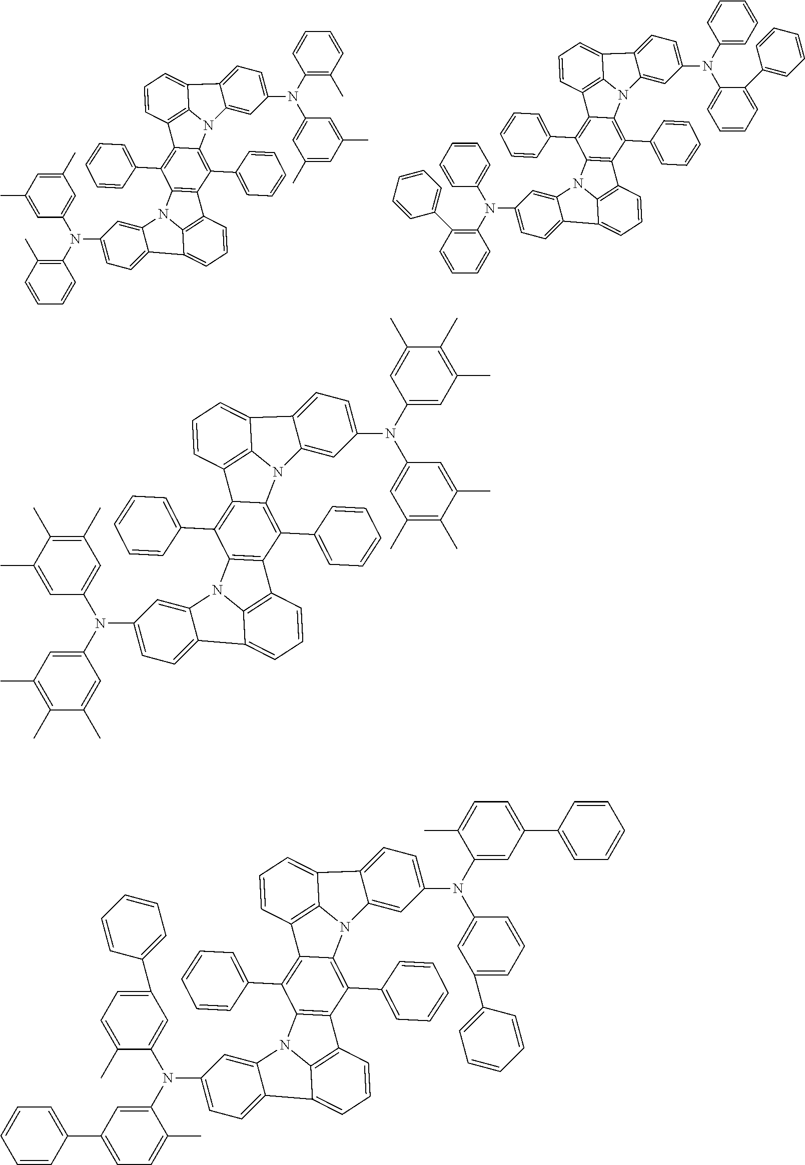

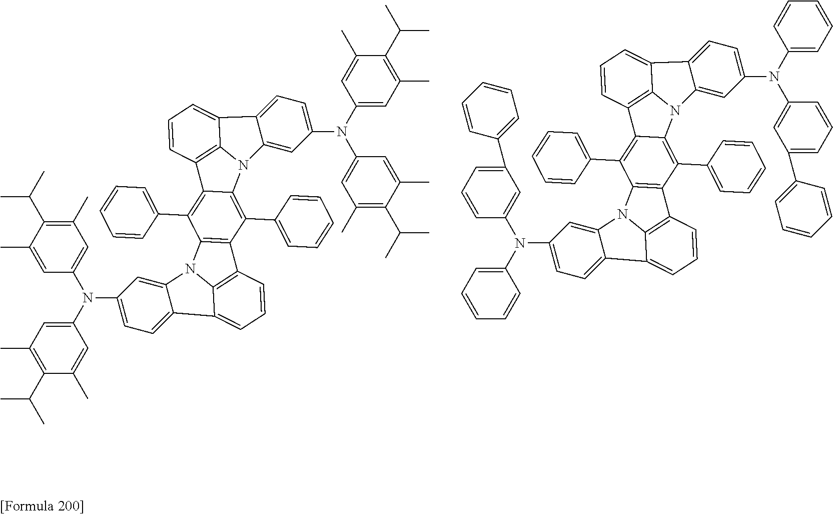

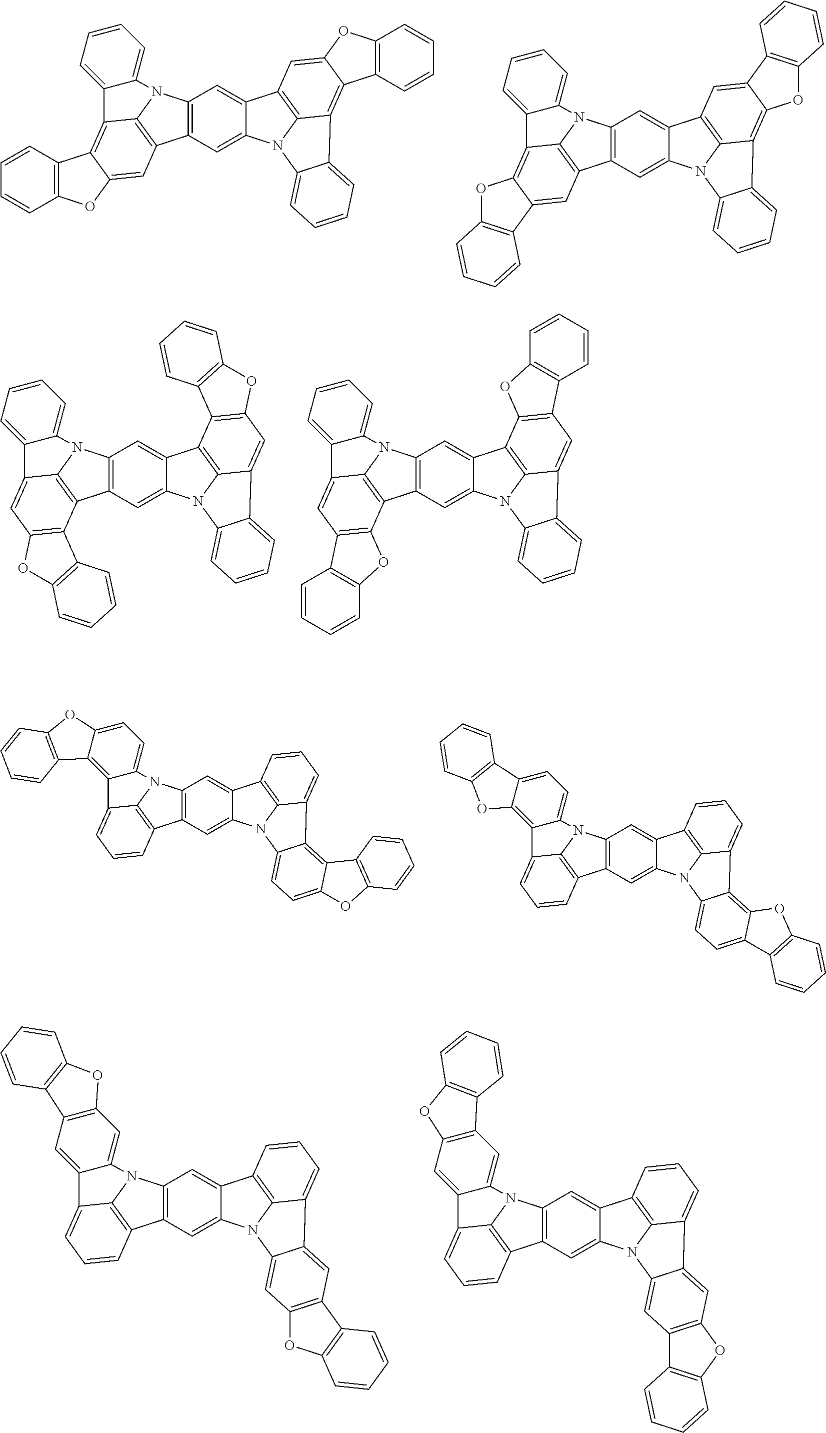

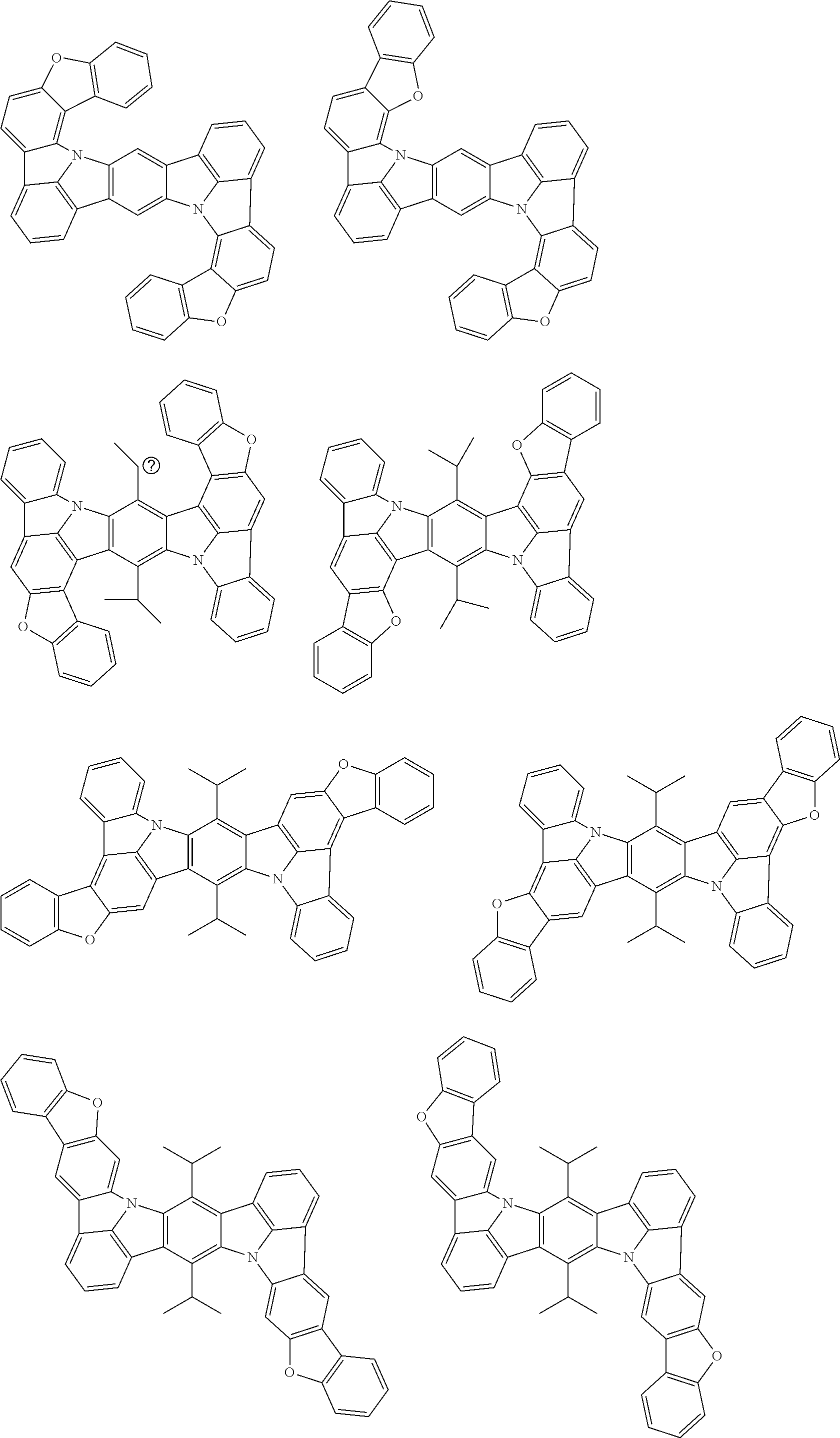

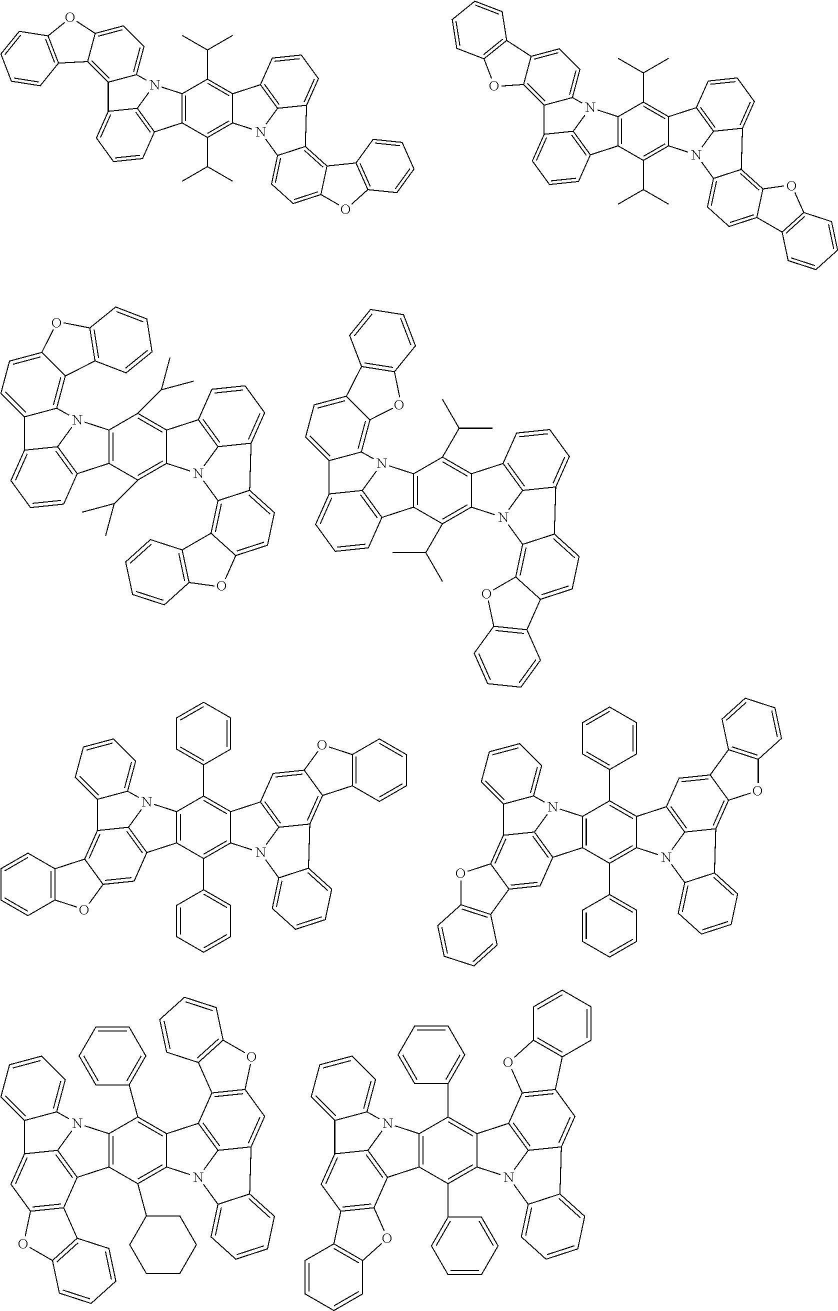

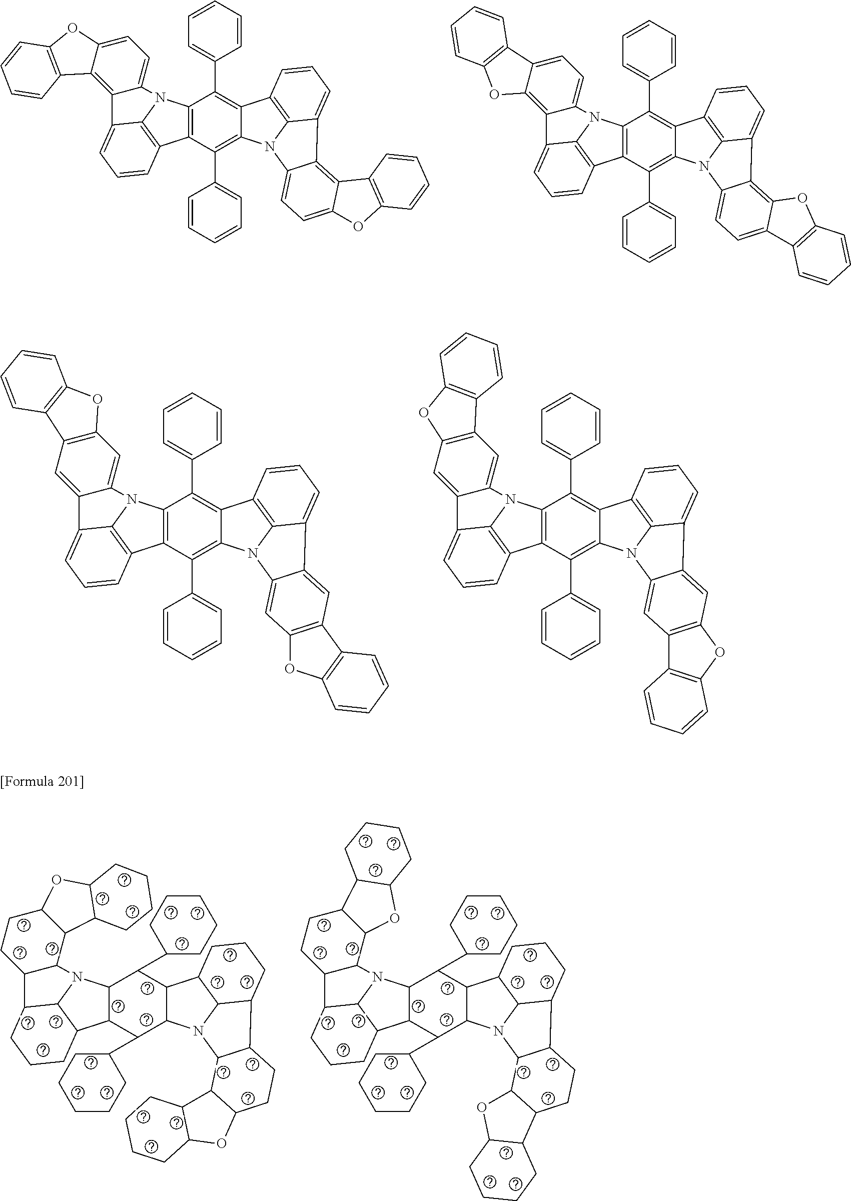

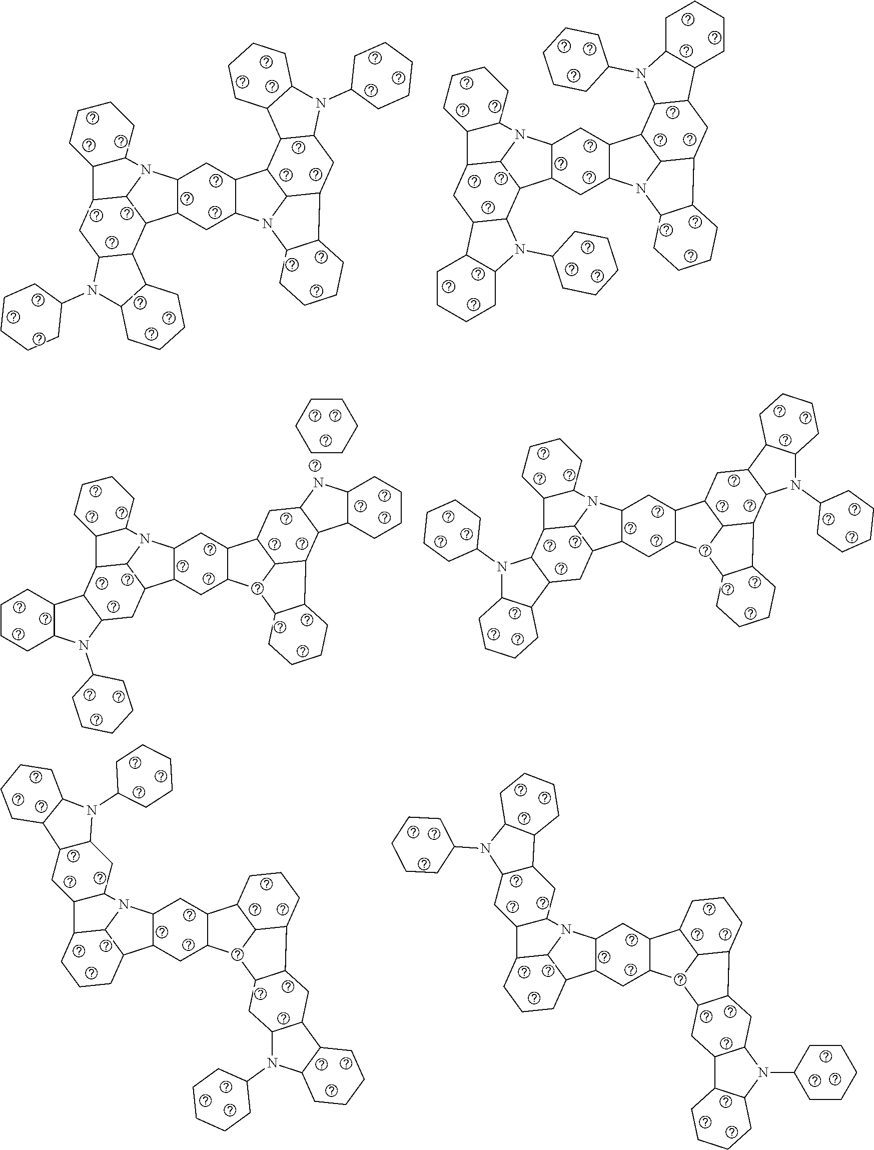

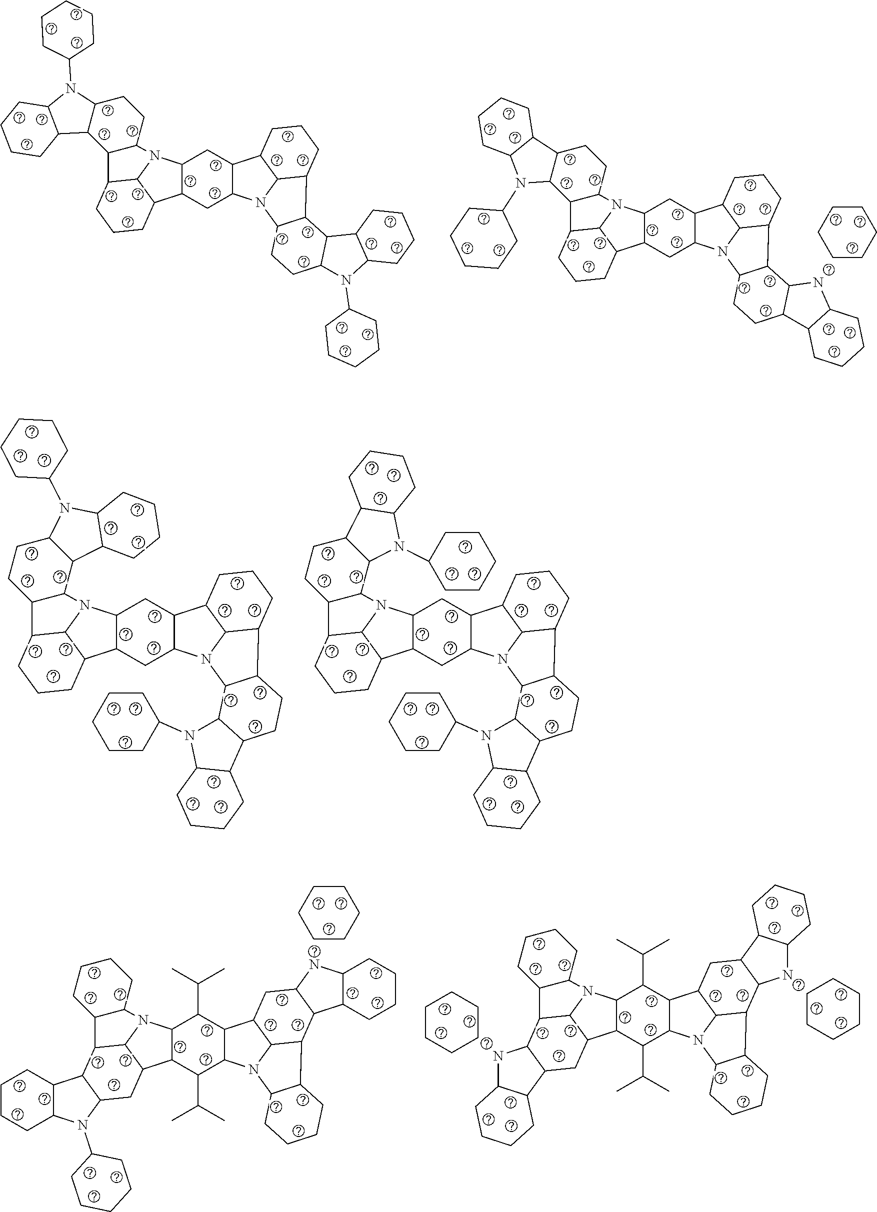

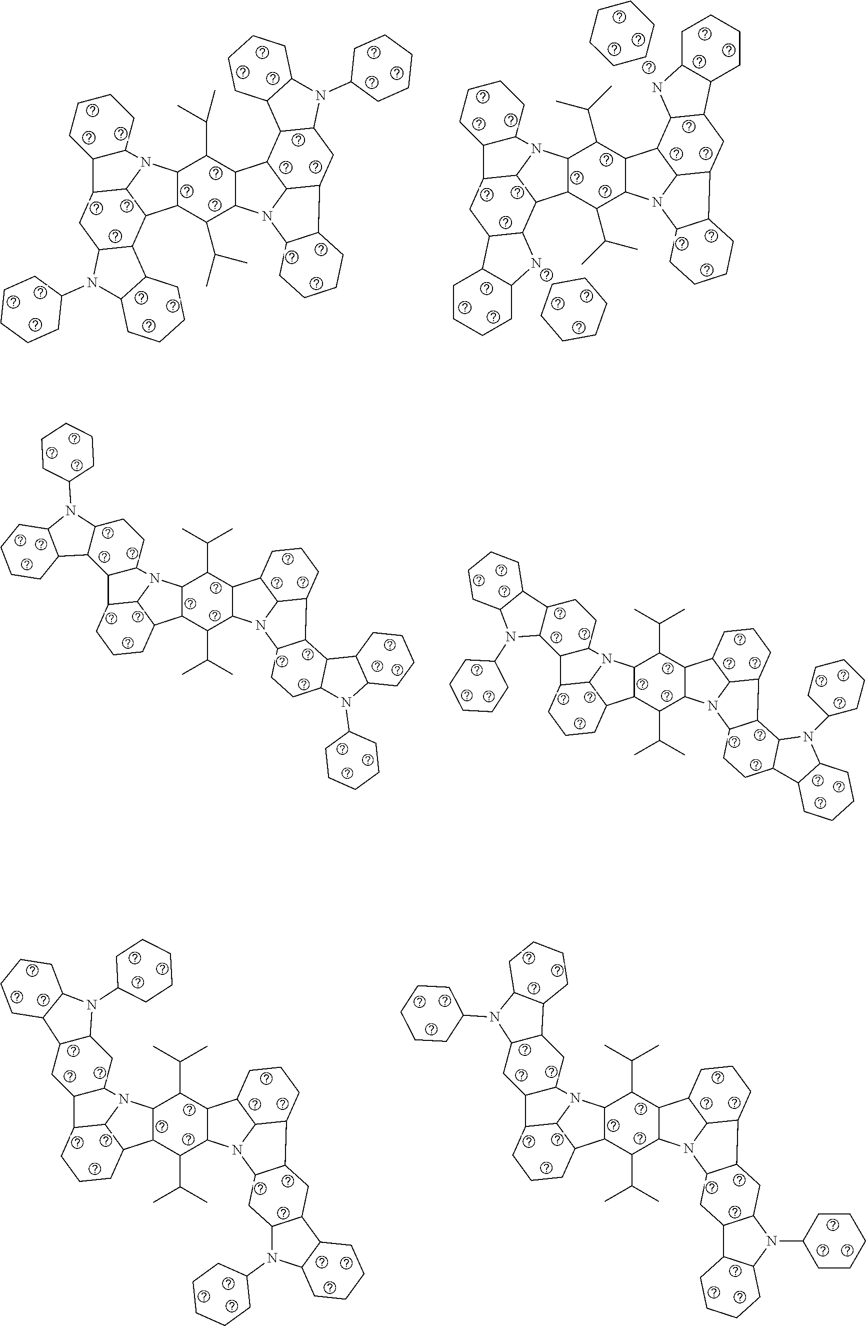

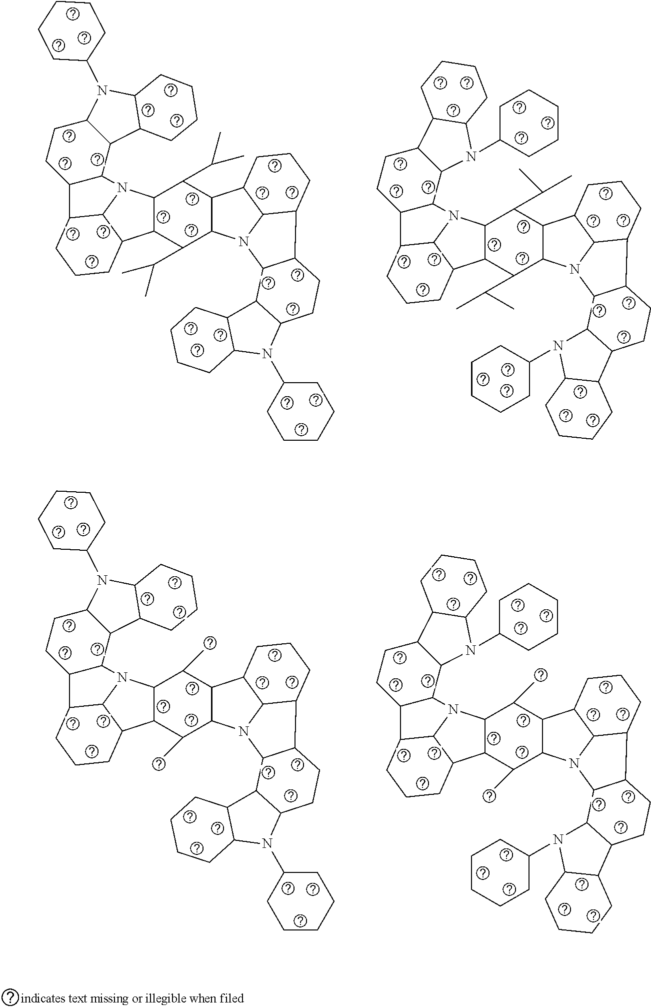

In the formulae, Ph represents a phenyl group.

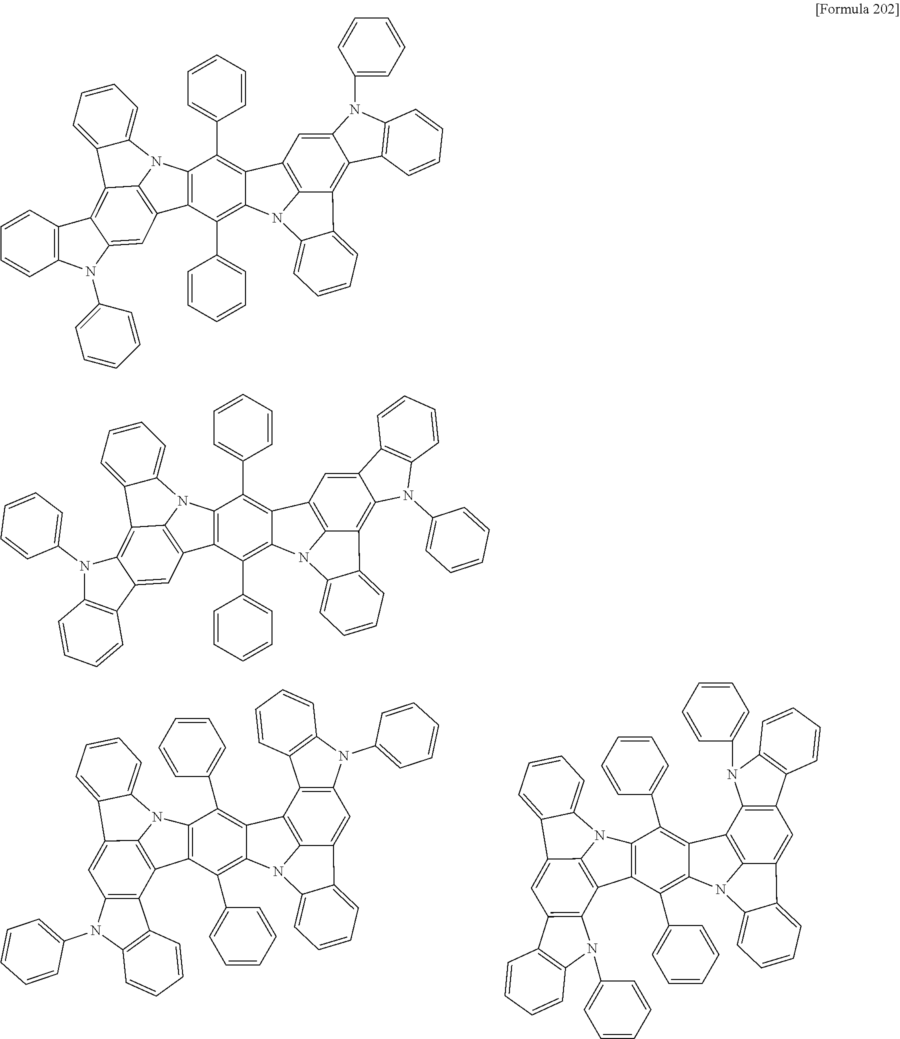

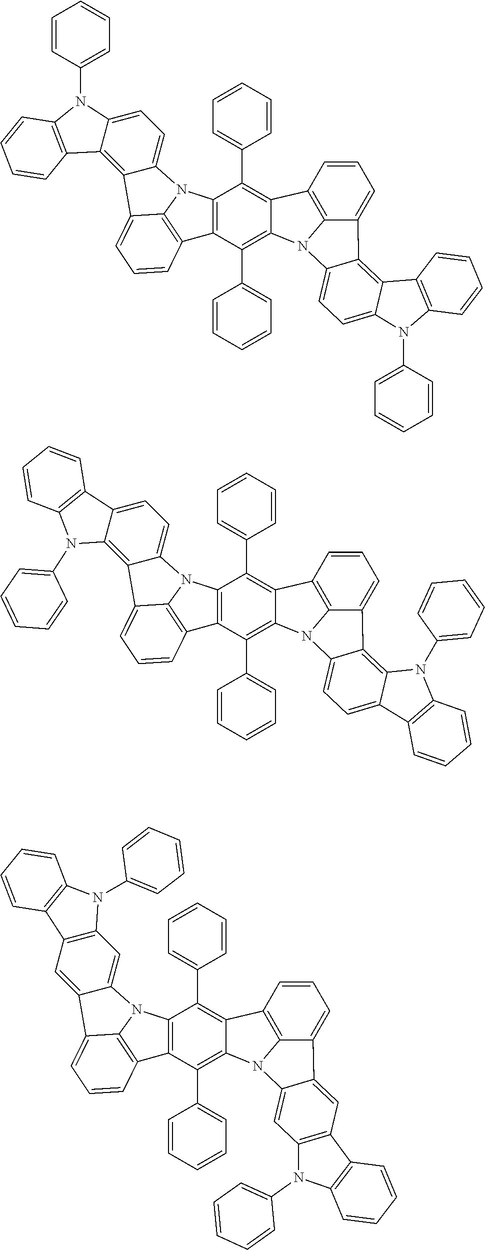 [SMILES: c1ccc(-c2c3c4cccc5c6c7c8ccccc8n(-c8ccccc8)c7ccc6n(c3c(-c3ccccc3)c3c6cccc7c8c9c%10ccccc%10n(-c%10ccccc%10)c9ccc8n(c23)c67)c45)cc1 ; c1ccc(-c2c3c4cccc5c6c7c(ccc6n(c3c(-c3ccccc3)c3c6cccc8c9c%10c(ccc9n(c23)c68)c2ccccc2n%10-c2ccccc2)c45)c2ccccc2n7-c2ccccc2)cc1 ; c1ccc(-c2c3c4cccc5c6cc7c8ccccc8n(-c8ccccc8)c7cc6n(c3c(-c3ccccc3)c3c6cccc7c8cc9c%10ccccc%10n(-c%10ccccc%10)c9cc8n(c23)c76)c54)cc1]

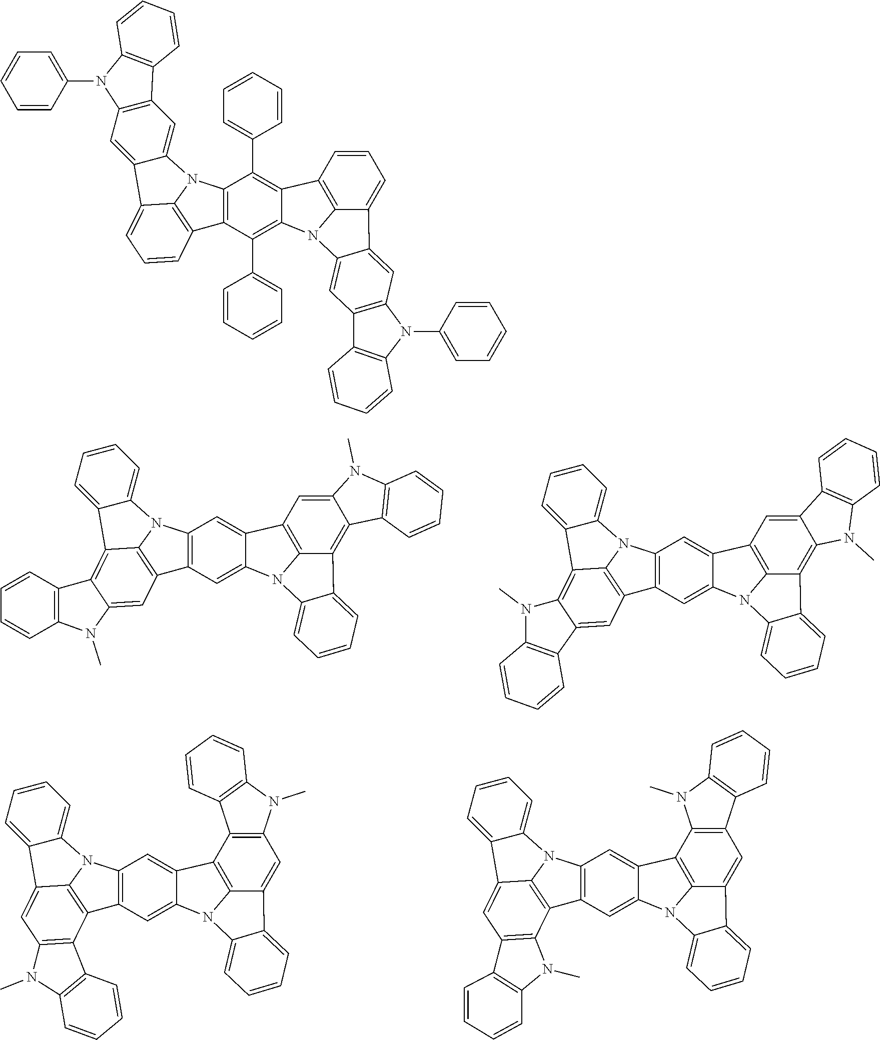

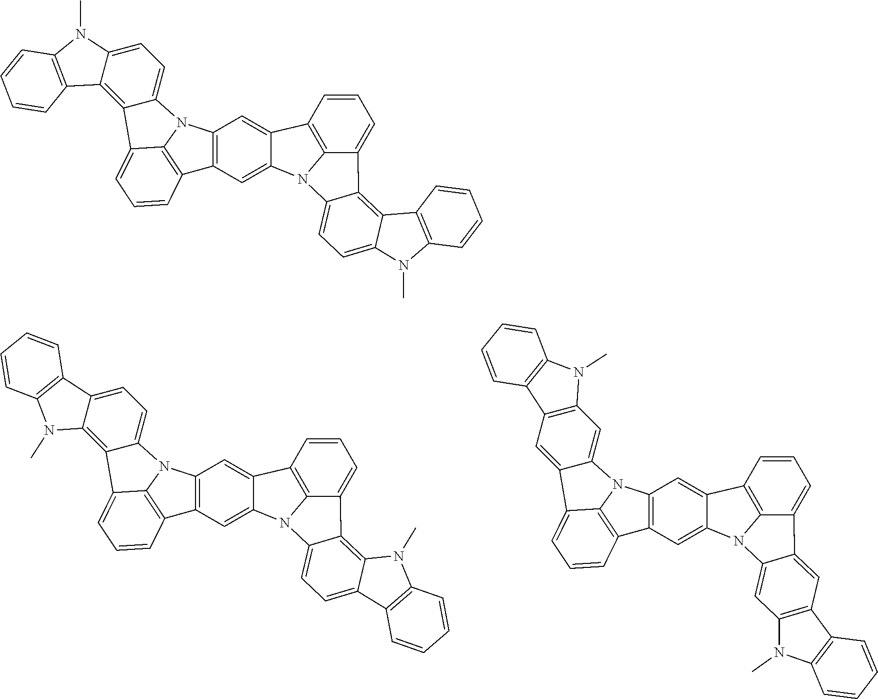 [SMILES: Cn1c2ccccc2c2c3c4cccc5c6cc7c(cc6n(c3ccc21)c54)c1cccc2c3c4c5ccccc5n(C)c4ccc3n7c12 ; Cn1c2ccccc2c2ccc3c(c4cccc5c6cc7c(cc6n3c54)c3cccc4c5c6c(ccc5n7c34)c3ccccc3n6C)c21 ; Cn1c2ccccc2c2cc3c4cccc5c6cc7c(cc6n(c3cc21)c54)c1cccc2c3cc4c5ccccc5n(C)c4cc3n7c12]

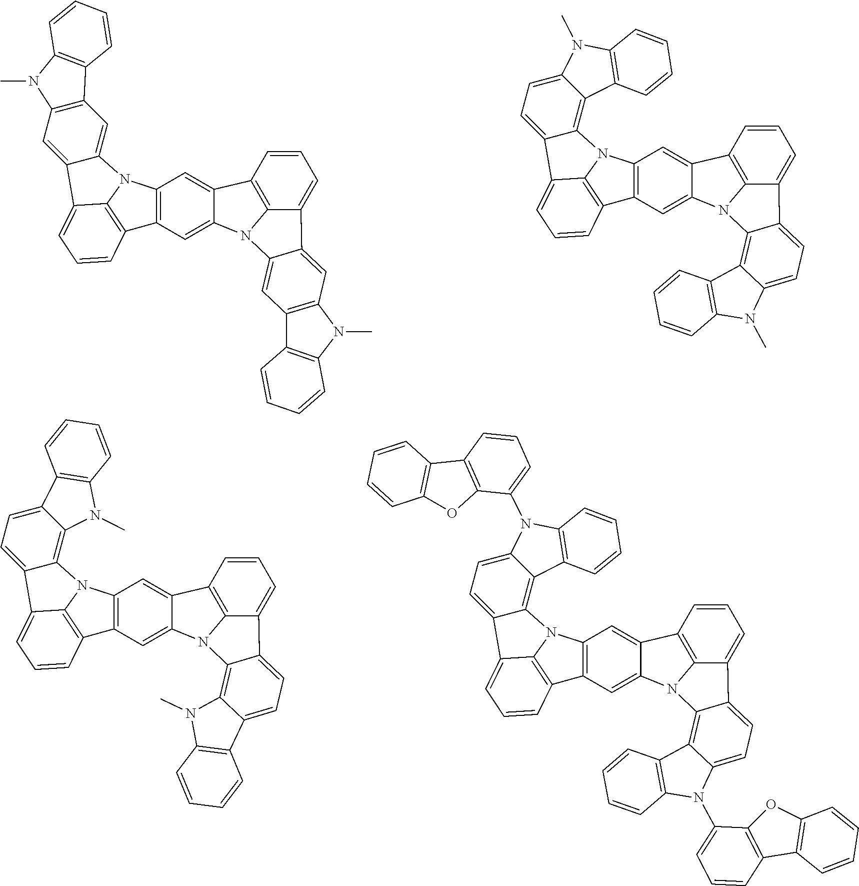

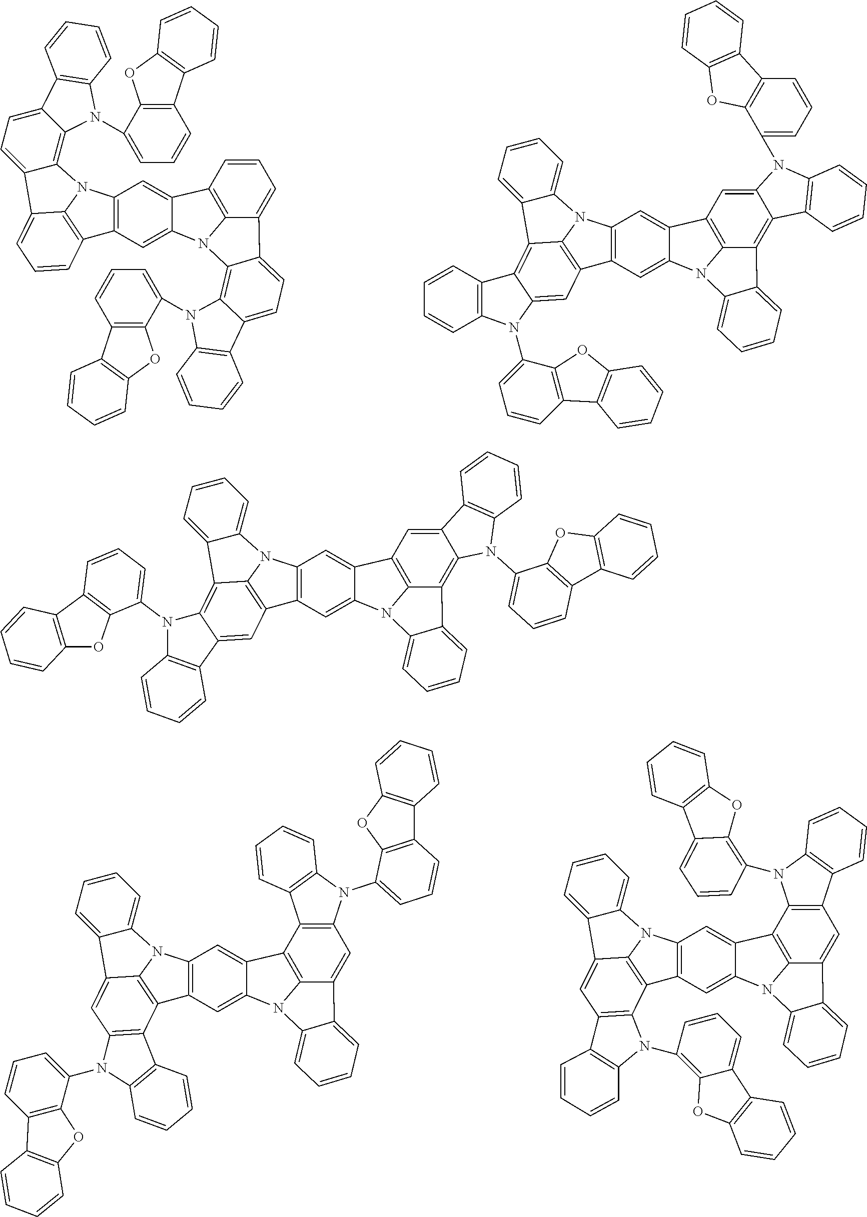

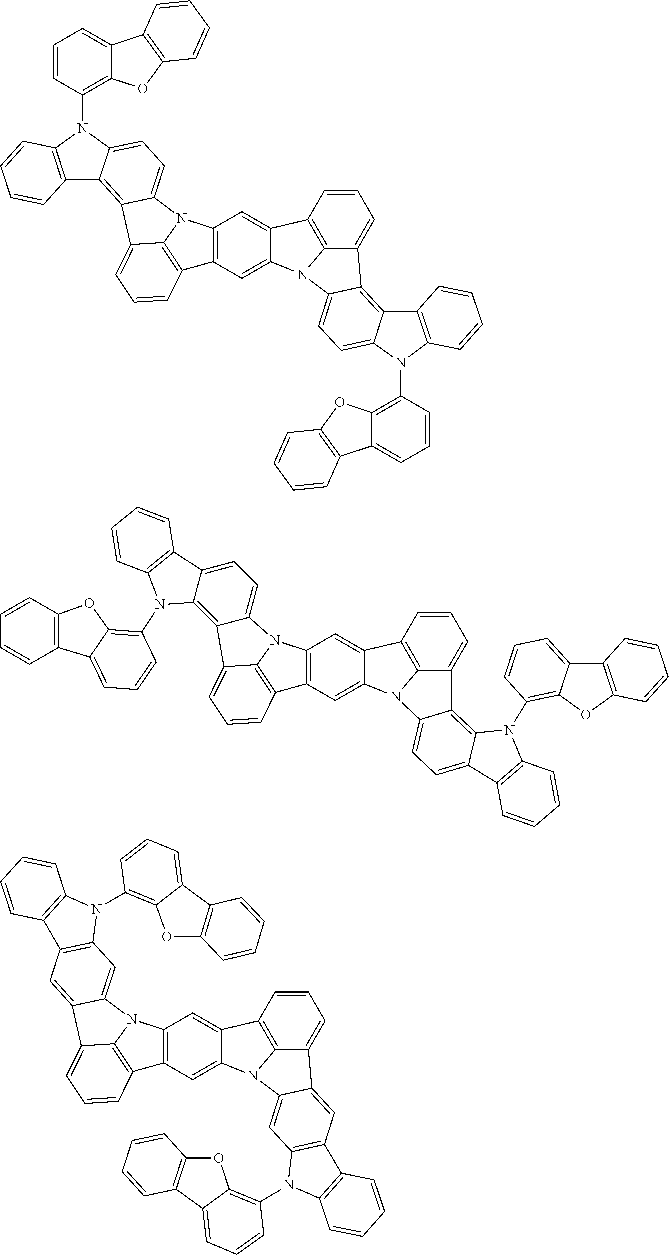 [SMILES: c1ccc2c(c1)oc1c(-n3c4ccccc4c4c5c6cccc7c8cc9c(cc8n(c5ccc43)c76)c3cccc4c5c6c7ccccc7n(-c7cccc8c7oc7ccccc78)c6ccc5n9c34)cccc12 ; c1ccc2c(c1)oc1c(-n3c4ccccc4c4ccc5c(c6cccc7c8cc9c(cc8n5c76)c5cccc6c7c8c(ccc7n9c56)c5ccccc5n8-c5cccc6c5oc5ccccc56)c43)cccc12 ; c1ccc2c(c1)oc1c(-n3c4ccccc4c4cc5c6cccc7c8cc9c(cc8n(c5cc43)c76)c3cccc4c5cc6c7ccccc7n(-c7cccc8c7oc7ccccc78)c6cc5n9c34)cccc12]

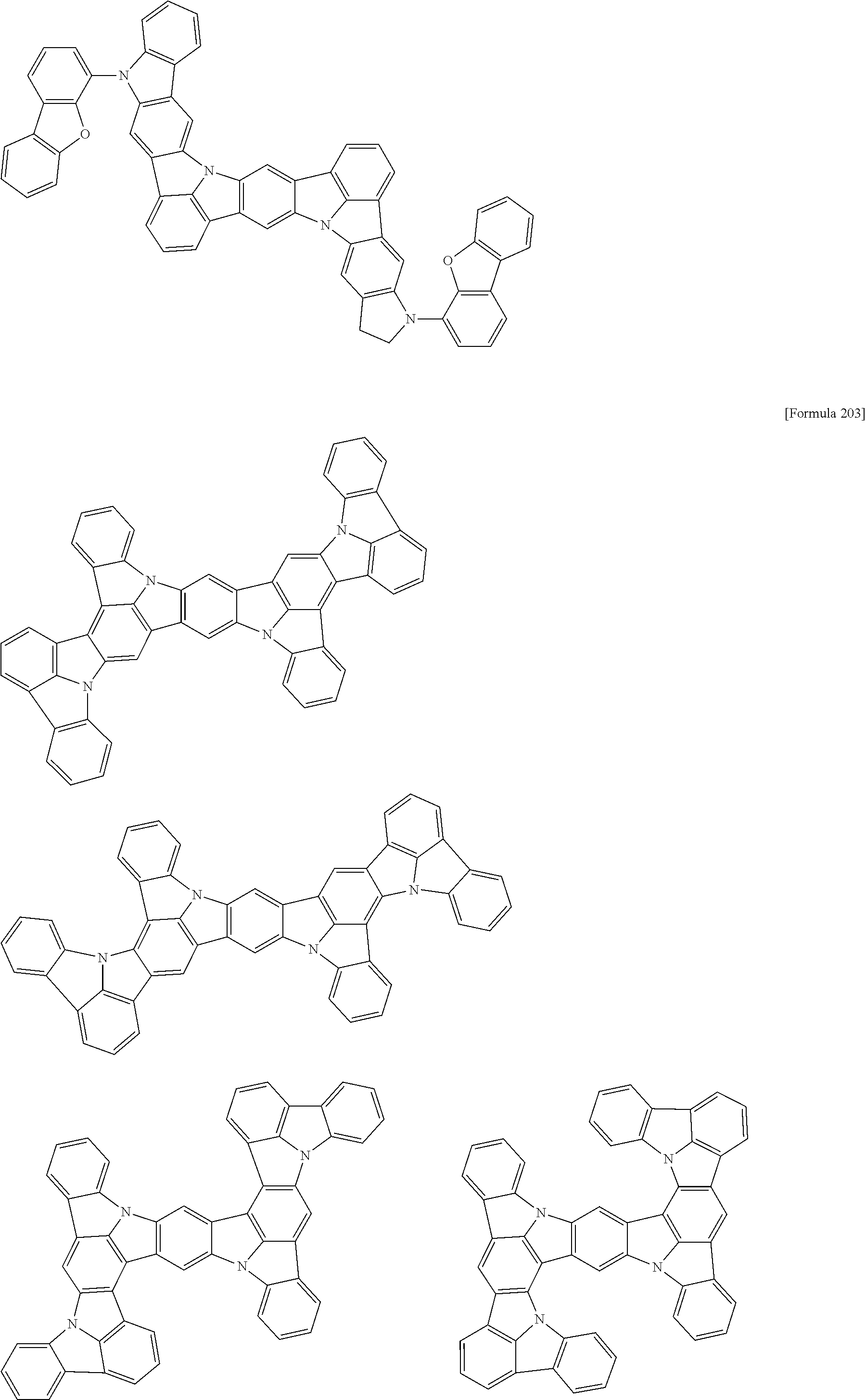

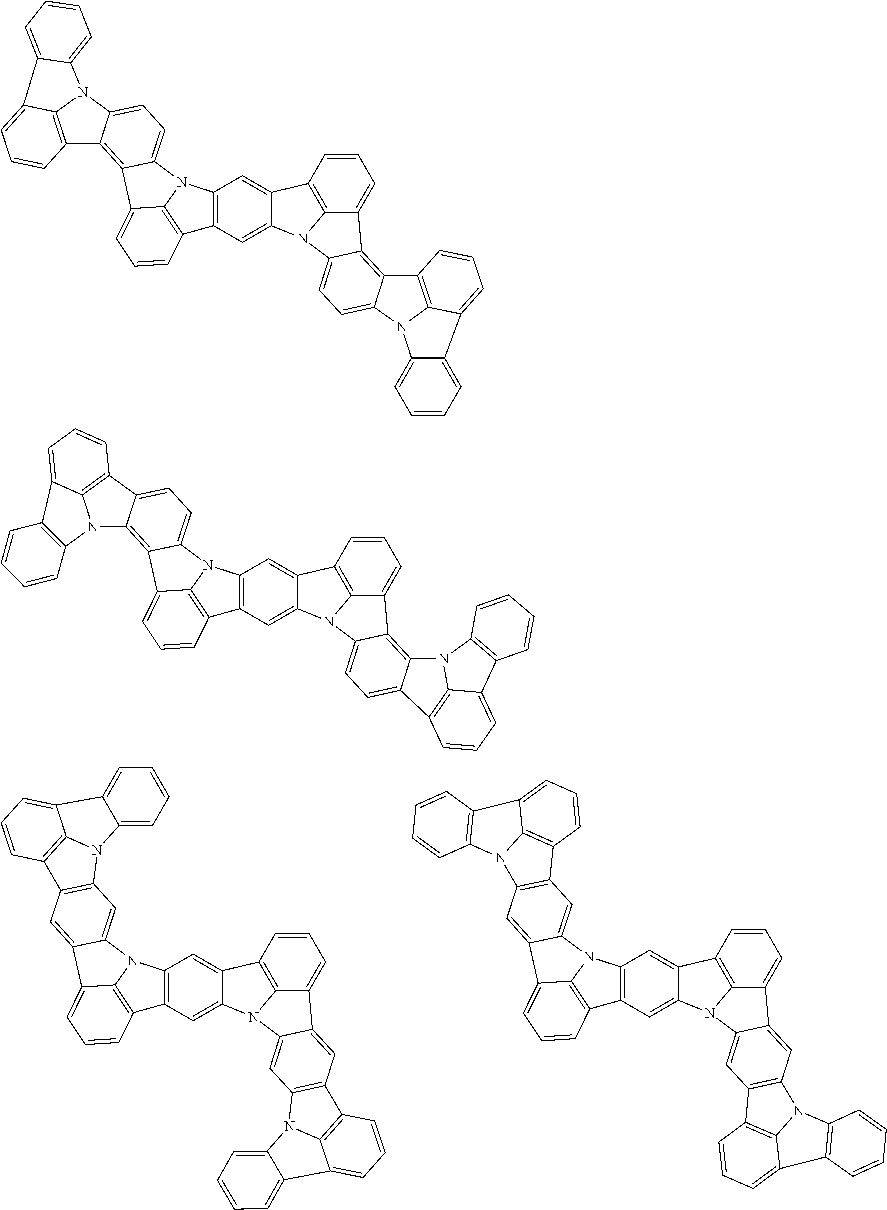

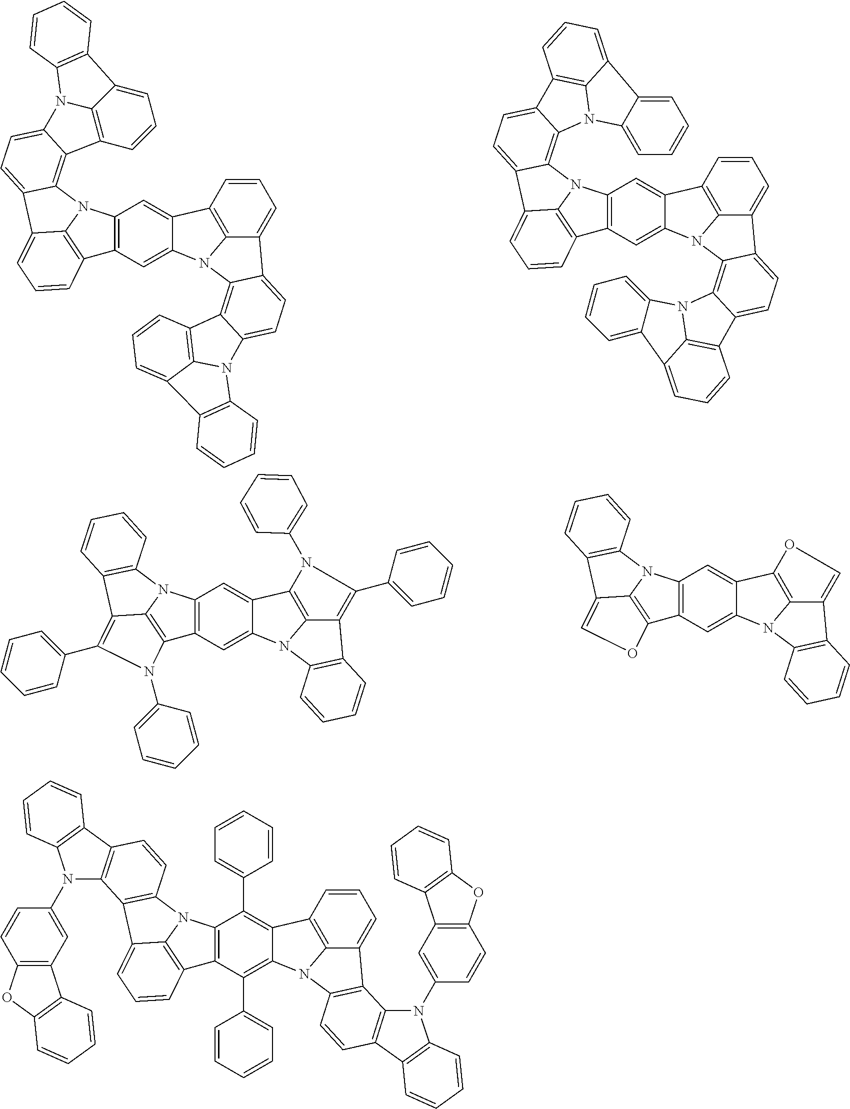

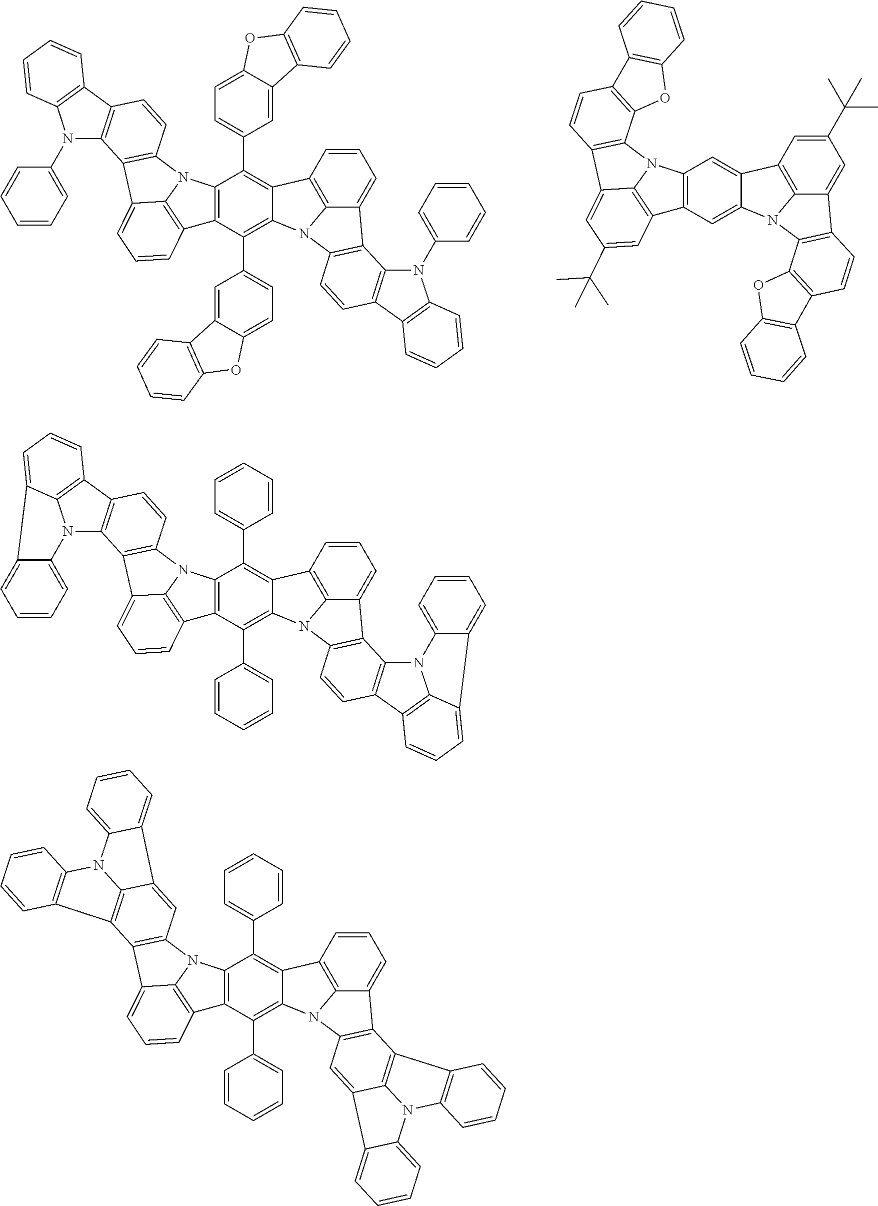

### Compound Represented by Formula (6)

The compound represented by the formula (6) will be described.

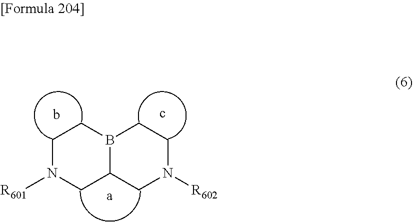

In the formula (6):

a ring, b ring and c ring are each independently a substituted or unsubstituted aromatic hydrocarbon ring having 6 to 50 ring carbon atoms, or a substituted or unsubstituted heterocycle having 5 to 50 ring atoms;

R₆₀₁ and R₆₀₂ are each independently bonded to the a ring, the b ring or the c ring to form a substituted or unsubstituted heterocycle, or not bonded to form no substituted or unsubstituted heterocycle; and

R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The a ring, b ring and c ring are each a ring (a substituted or unsubstituted aromatic hydrocarbon ring having 6 to 50 ring carbon atoms, or a substituted or unsubstituted heterocycle having 5 to 50 ring atoms) fused with the fused bicyclic structure formed of a boron atom and two nitrogen atoms at the center of the formula (6).

The “aromatic hydrocarbon ring” for the a, b, and c rings has the same structure as the compound formed by introducing a hydrogen atom to the “aryl group” described above.

Ring atoms of the “aromatic hydrocarbon ring” for the a ring include three carbon atoms on the fused bicyclic structure at the center of the formula (6).

Ring atoms of the “aromatic hydrocarbon ring” for the b ring and the c ring include two carbon atoms on a fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the a, b, and c rings has the same structure as the compound formed by introducing a hydrogen atom to the “heterocyclic group” described above.

Ring atoms of the “heterocycle” for the a ring include three carbon atoms on the fused bicyclic structure at the center of the formula (6). Ring atoms of the “heterocycle” for the b ring and the c ring include two carbon atoms on a fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ are optionally each independently bonded with the a ring, b ring, or c ring to form a substituted or unsubstituted heterocycle. The “heterocycle” in this arrangement includes the nitrogen atom on the fused bicyclic structure at the center of the formula (6). The heterocycle in the above arrangement optionally includes a hetero atom other than the nitrogen atom. R₆₀₁ and R₆₀₂ bonded with the a ring, b ring, or c ring specifically means that atoms forming R₆₀₁ and R₆₀₂ are bonded with atoms forming the a ring, b ring, or c ring. For instance, R₆₀₁ may be bonded to the a ring to form a bicyclic (or tri-or-more cyclic) fused nitrogen-containing heterocycle, in which the ring including R₆₀₁ and the a ring are fused. Specific examples of the nitrogen-containing heterocycle include a compound corresponding to the nitrogen-containing bi(or-more)cyclic fused heterocyclic group in the specific example group G2.

The same applies to R₆₀₁ bonded with the b ring, R₆₀₂ bonded with the a ring, and R₆₀₂ bonded with the c ring.

In an exemplary embodiment, the a ring, b ring and c ring in the formula (6) are each independently a substituted or unsubstituted aromatic hydrocarbon ring having 6 to 50 ring carbon atoms.

In an exemplary embodiment, the a ring, b ring and c ring in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms, preferably a substituted or unsubstituted aryl group having 6 to 50 ring carbon atoms.

In an exemplary embodiment, the compound represented by the formula (6) is a compound represented by a formula (62) below.

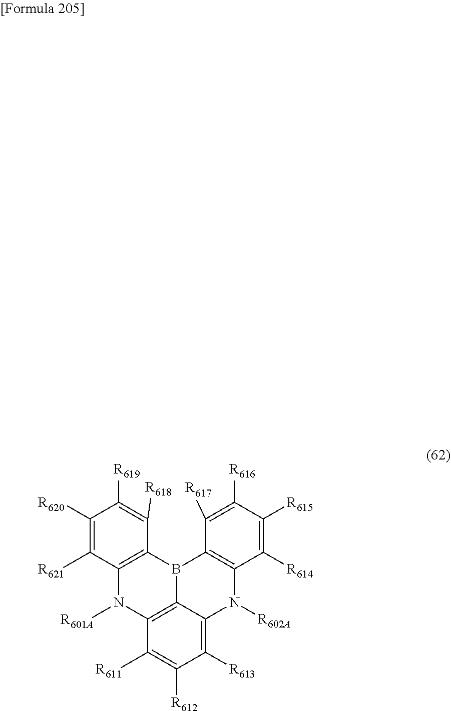

In the formula (62):

R_{601A} is bonded with at least one of R₆₁₁ or R₆₂₁ to form a substituted or unsubstituted heterocycle, or not bonded to form no substituted or unsubstituted heterocycle;

R_{602A} is bonded with at least one of R₆₁₃ or R₆₁₄ to form a substituted or unsubstituted heterocycle, or not bonded to form no substituted or unsubstituted heterocycle;

R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

at least one combination of adjacent two or more of R₆₁₁ to R₆₂₁ are mutually bonded to form a substituted or unsubstituted monocyclic ring, mutually bonded to form a substituted or unsubstituted fused ring, or not mutually bonded; and

R₆₁₁ to R₆₂₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the a ring are fused.

Specific examples of the nitrogen-containing heterocycle include a compound corresponding to the nitrogen-containing bi(or-more)cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ are optionally mutually bonded to form a substituted or unsubstituted monocyclic ring, or mutually bonded to form a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring, respectively.

In an exemplary embodiment, R₆₁₁ to R₆₂₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₁₁ to R₆₂₁ not contributing to ring formation are each independently a hydrogen atom, or a substituted or unsubstituted alkyl group having 1 to 50 carbon atoms.

In an exemplary embodiment, R₆₁₁ to R₆₂₁ not contributing to ring formation are each independently a hydrogen atom, or a substituted or unsubstituted alkyl group having 1 to 50 carbon atoms, and

at least one of R₆₁₁ to R₆₂₁ is a substituted or unsubstituted alkyl group having 1 to 50 carbon atoms.

In an exemplary embodiment, the compound represented by the formula (62) is a compound represented by a formula (63) below.

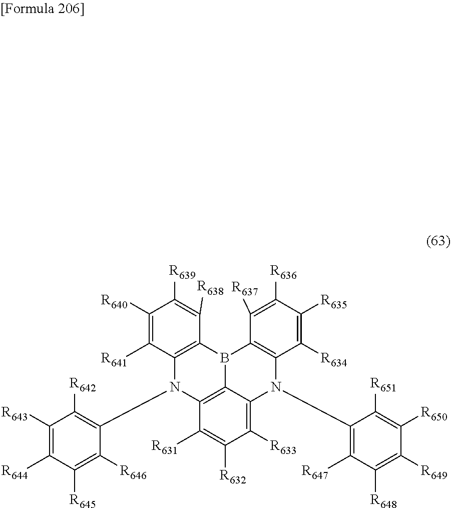

In the formula (63):

R₆₃₁ is bonded with R₆₄₆ to form a substituted or unsubstituted heterocycle, or not bonded to form no substituted or unsubstituted heterocycle;

R₆₃₃ is bonded with R₆₄₇ to form a substituted or unsubstituted heterocycle, or not bonded to form no substituted or unsubstituted heterocycle;

R₆₃₄ is bonded with R₆₅₁ to form a substituted or unsubstituted heterocycle, or not bonded to form no substituted or unsubstituted heterocycle;

R₆₄₁ is bonded with R₆₄₂ to form a substituted or unsubstituted heterocycle, or not bonded to form no substituted or unsubstituted heterocycle;

at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded; and

R₆₃₁ to R₆₅₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

R₆₃₁ are optionally mutually bonded with R₆₄₆ to form a substituted or unsubstituted heterocycle. For instance, R₆₃₁ and R₆₄₆ are optionally bonded with each other to form a tri-or-more cyclic fused nitrogen-containing heterocycle, in which a benzene ring bonded with R₆₄₆, a ring including a nitrogen atom, and a benzene ring corresponding to the a ring are fused. Specific examples of the nitrogen-containing heterocycle include a compound corresponding to the nitrogen-containing tri(-or-more)cyclic fused heterocyclic group in the specific example group G2. The same applies to R₆₃₃ bonded with R₆₄₇, R₆₃₄ bonded with R₆₅₁, and R₆₄₁ bonded with R₆₄₂.

In an exemplary embodiment, R₆₃₁ to R₆₅₁ not contributing to ring formation are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₆₃₁ to R₆₅₁ not contributing to ring formation are each independently a hydrogen atom, or a substituted or unsubstituted alkyl group having 1 to 50 carbon atoms.

In an exemplary embodiment, R₆₃₁ to R₆₅₁ not contributing to ring formation are each independently a hydrogen atom, or a substituted or unsubstituted alkyl group having 1 to 50 carbon atoms, and

at least one of R₆₃₁ to R₆₅₁ is a substituted or unsubstituted alkyl group having 1 to 50 carbon atoms.

In an exemplary embodiment, the compound represented by the formula (63) is a compound represented by a formula (63A) below.

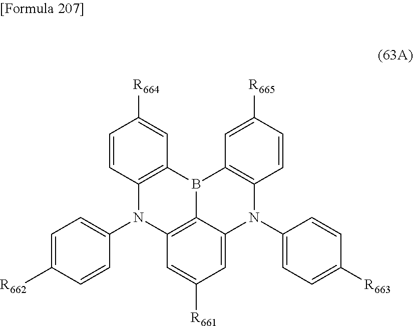

In the formula (63A):

R₆₆₁ is a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms, and

R₆₆₂ to R₆₆₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms, or a substituted or unsubstituted aryl group having 6 to 50 ring carbon atoms.

In an exemplary embodiment, R₆₆₁ to R₆₆₅ are each independently a substituted or unsubstituted alkyl group having 1 to 50 carbon atoms.

In an exemplary embodiment, the compound represented by the formula (63) is a compound represented by a formula (63B) below.

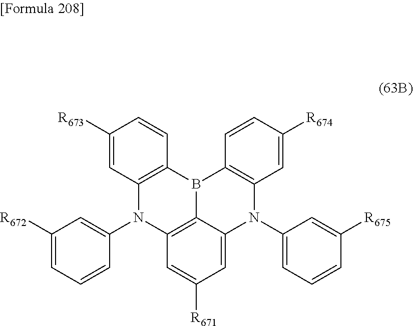

In the formula (63B):

R₆₇₁ and R₆₇₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms; and

R₆₇₃ to R₆₇₅ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63B′) below.

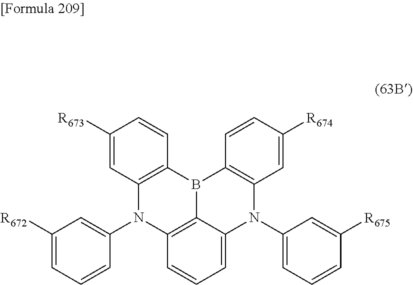

In the formula (63B′), R₆₇₂ to R₆₇₅ each independently represent the same as R₆₇₂ to R₆₇₅ in the formula (63B).

In an exemplary embodiment, at least one of R₆₇₁ to R₆₇₅ is a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —N(R₉₀₆)(R₉₀₇), or a substituted or unsubstituted aryl group having 6 to 50 ring carbon atoms.

In an exemplary embodiment: R₆₇₂ is a hydrogen atom, a substituted or unsubstituted alkyl group having 1 to 50 carbon atoms, a group represented by —N(R₉₀₆)(R₉₀₇), or a substituted or unsubstituted aryl group having 6 to 50 ring carbon atoms; and

R₆₇₁, and R₆₇₃ to R₆₇₅ are each independently a substituted or unsubstituted alkyl group having 1 to 50 carbon atoms, a group represented by —N(R₉₀₆)(R₉₀₇), or a substituted or unsubstituted aryl group having 6 to 50 ring carbon atoms.

In an exemplary embodiment, the compound represented by the formula (63) is a compound represented by a formula (63C) below.

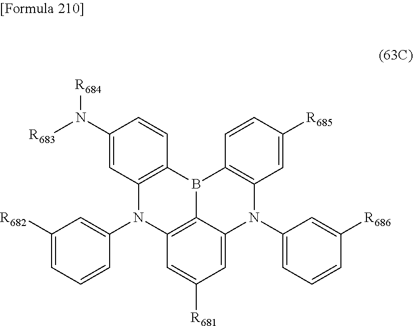

In the formula (63C): R₆₈₁ and R₆₈₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms; and

R₆₈₃ to R₆₈₆ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, or a substituted or unsubstituted aryl group having 6 to 50 ring carbon atoms.

In an exemplary embodiment, the compound represented by the formula (63) is a compound represented by a formula (63C′) below.

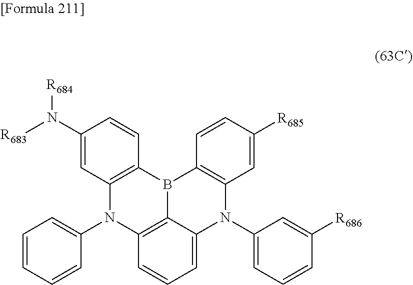

In the formula (63C′), R₆₈₃ to R₆₈₆ each independently represent the same as R₆₈₃ to R₆₈₆ in the formula (63C).

In an exemplary embodiment, R₆₈₁ to R₆₈₆ are each independently a substituted or unsubstituted alkyl group having 1 to 50 carbon atoms, or a substituted or unsubstituted aryl group having 6 to 50 ring carbon atoms.

In an exemplary embodiment, R₆₈₁ to R₆₈₆ are each independently a substituted or unsubstituted aryl group having 6 to 50 ring carbon atoms.

The compound represented by the formula (6) is producible by initially bonding the a ring, b ring and c ring with linking groups (a group including N—R₆₀₁ and a group including N—R₆₀₂) to form an intermediate (first reaction), and bonding the a ring, b ring and c ring with a linking group (a group including a boron atom) to form a final product (second reaction). In the first reaction, an amination reaction (e.g. Buchwald-Hartwig reaction) is applicable. In the second reaction, Tandem Hetero-Friedel-Crafts Reactions or the like is applicable.

Specific examples of the compound represented by the formula (6) are shown below. It should however be noted that these specific examples are merely exemplary and do not limit the compound represented by the formula (6).

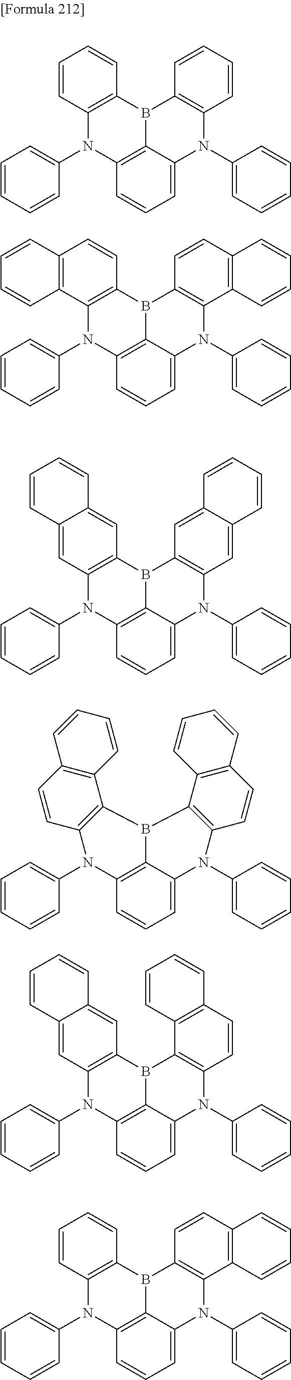

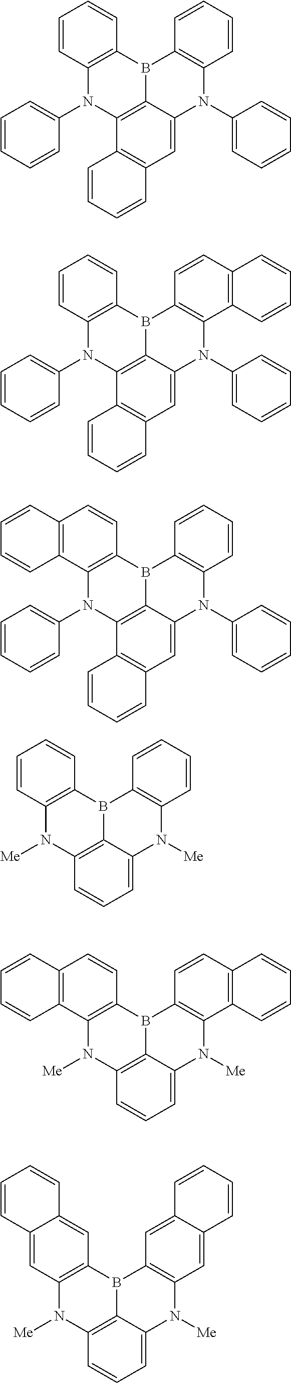

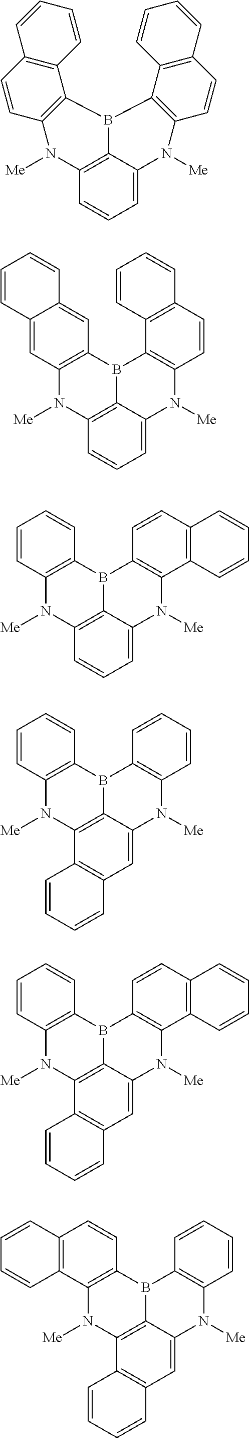

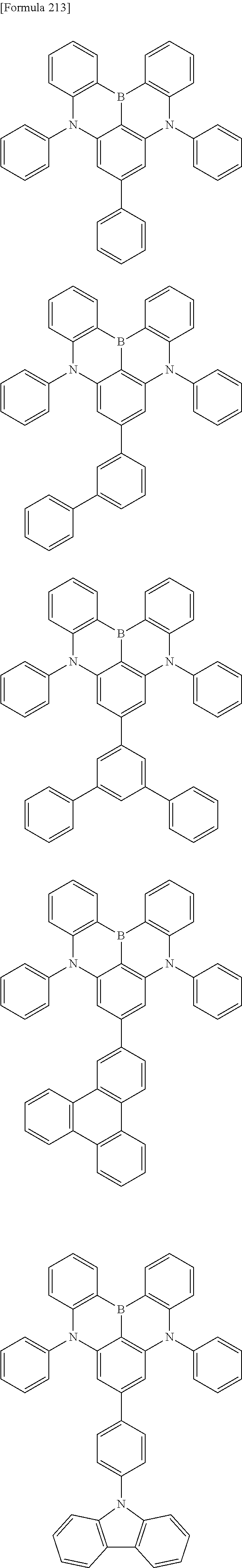

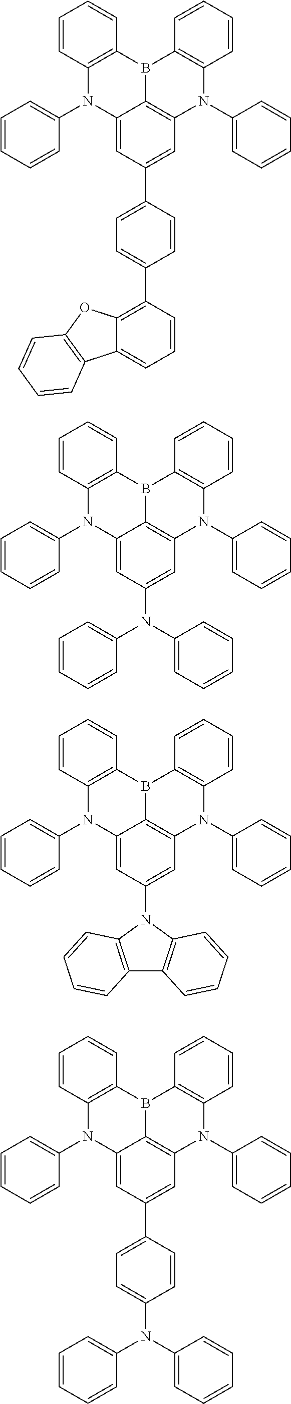

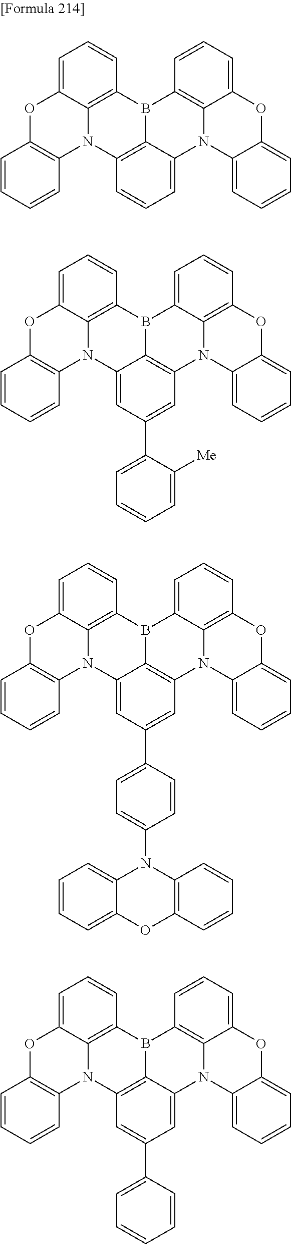 [SMILES: c1ccc2c(c1)Oc1cccc3c1N2c1cccc2c1B3c1cccc3c1N2c1ccccc1O3 ; Cc1ccccc1-c1cc2c3c(c1)N1c4ccccc4Oc4cccc(c41)B3c1cccc3c1N2c1ccccc1O3 ; c1ccc2c(c1)Oc1ccccc1N2c1ccc(-c2cc3c4c(c2)N2c5ccccc5Oc5cccc(c52)B4c2cccc4c2N3c2ccccc2O4)cc1 ; c1ccc(-c2cc3c4c(c2)N2c5ccccc5Oc5cccc(c52)B4c2cccc4c2N3c2ccccc2O4)cc1]

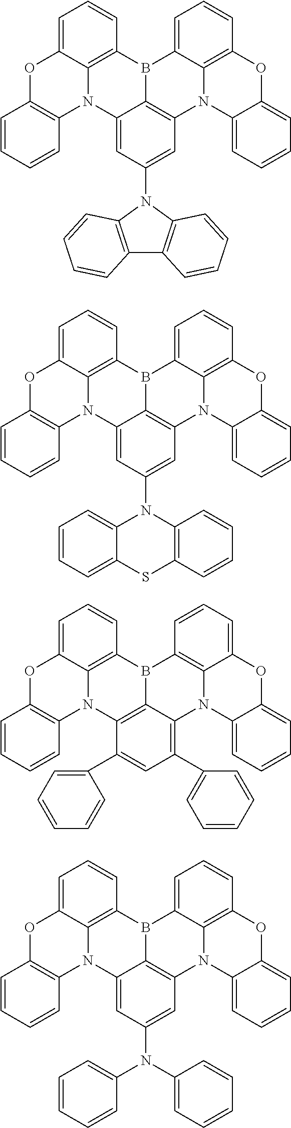

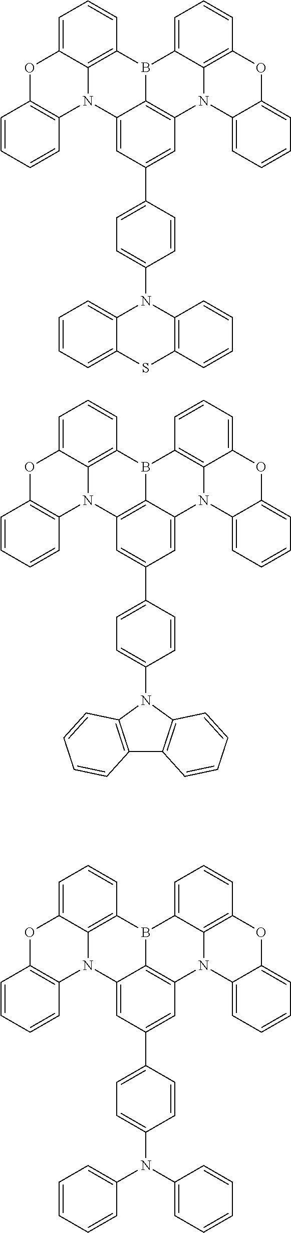

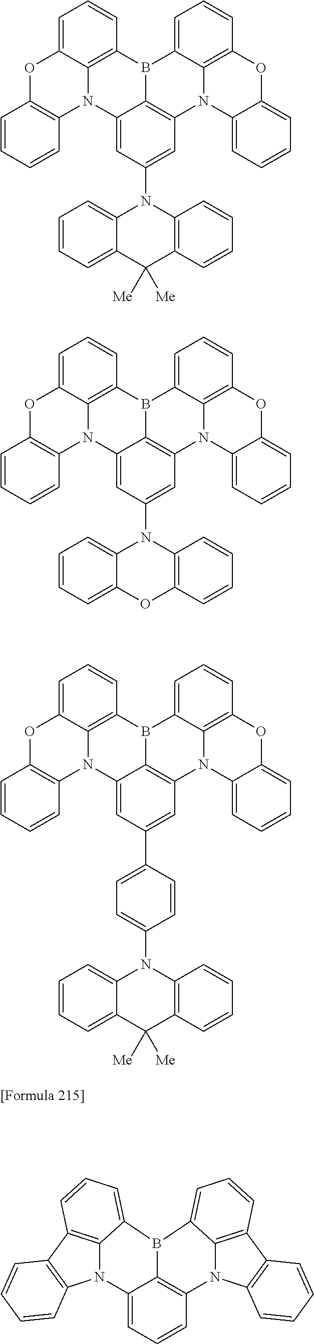 [SMILES: CC1(C)c2ccccc2N(c2cc3c4c(c2)N2c5ccccc5Oc5cccc(c52)B4c2cccc4c2N3c2ccccc2O4)c2ccccc21 ; c1ccc2c(c1)Oc1ccccc1N2c1cc2c3c(c1)N1c4ccccc4Oc4cccc(c41)B3c1cccc3c1N2c1ccccc1O3 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21 ; c1cc2c3c(c1)-n1c4ccccc4c4cccc(c41)B3c1cccc3c4ccccc4n-2c13]

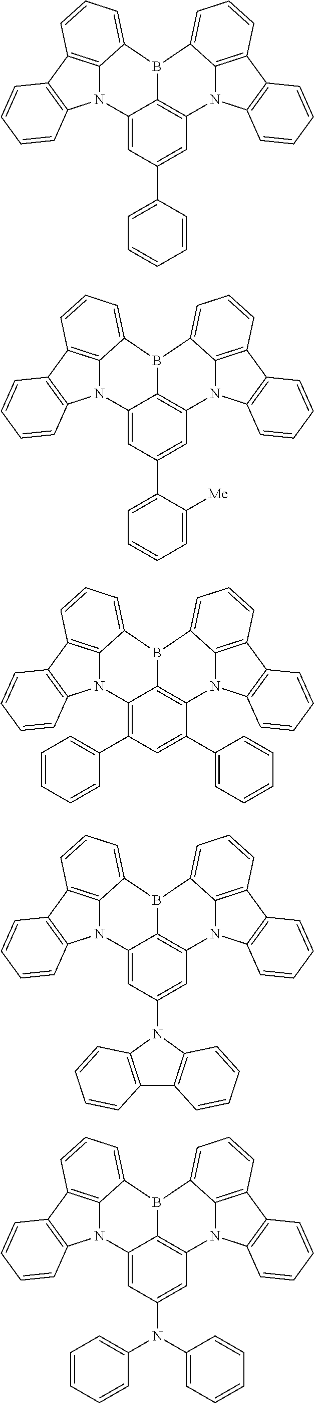

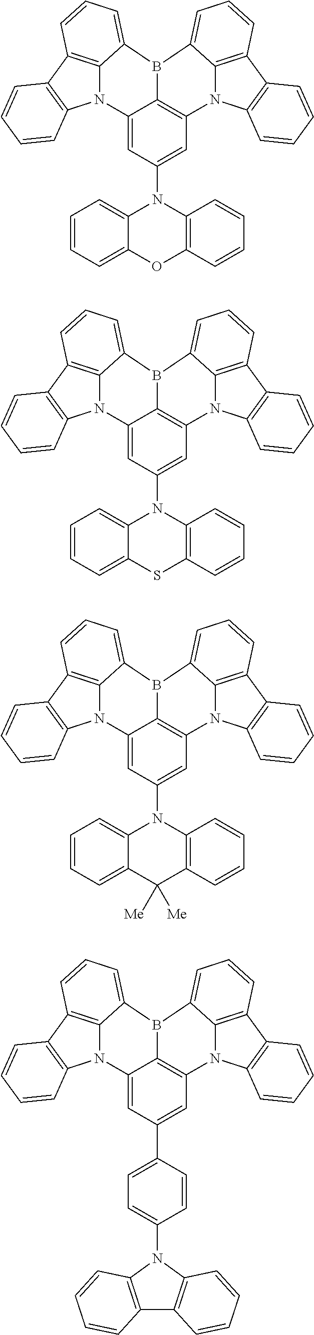

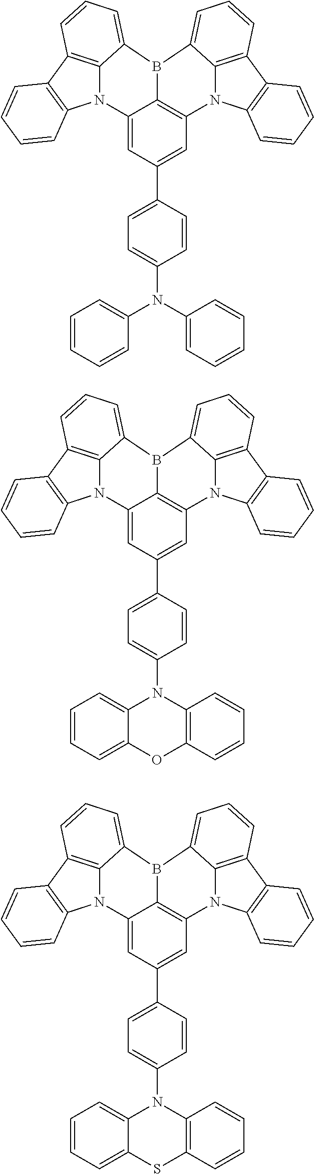

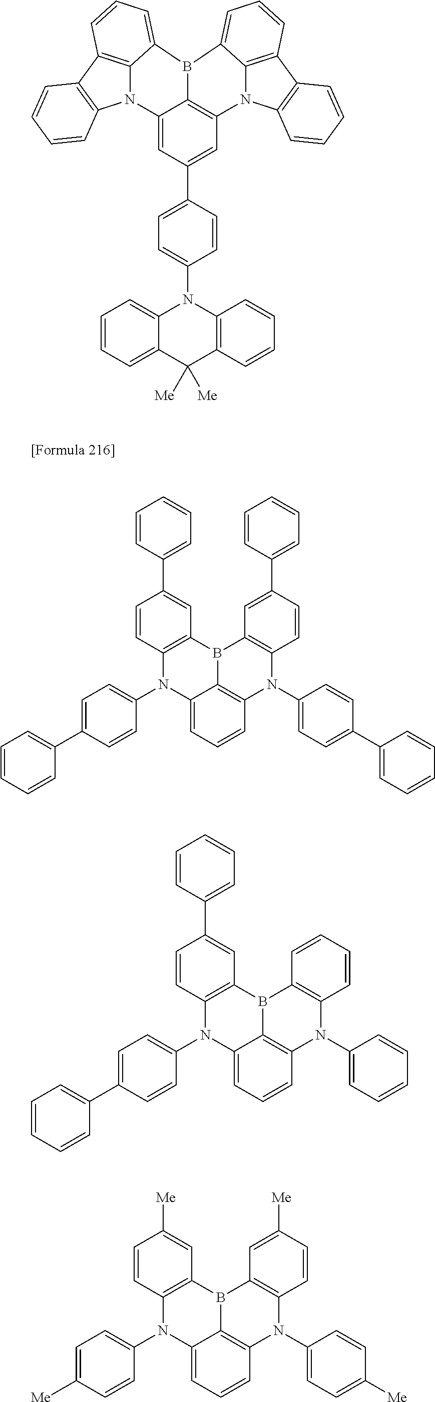 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; Cc1ccc(N2c3ccc(C)cc3B3c4cc(C)ccc4N(c4ccc(C)cc4)c4cccc2c43)cc1]

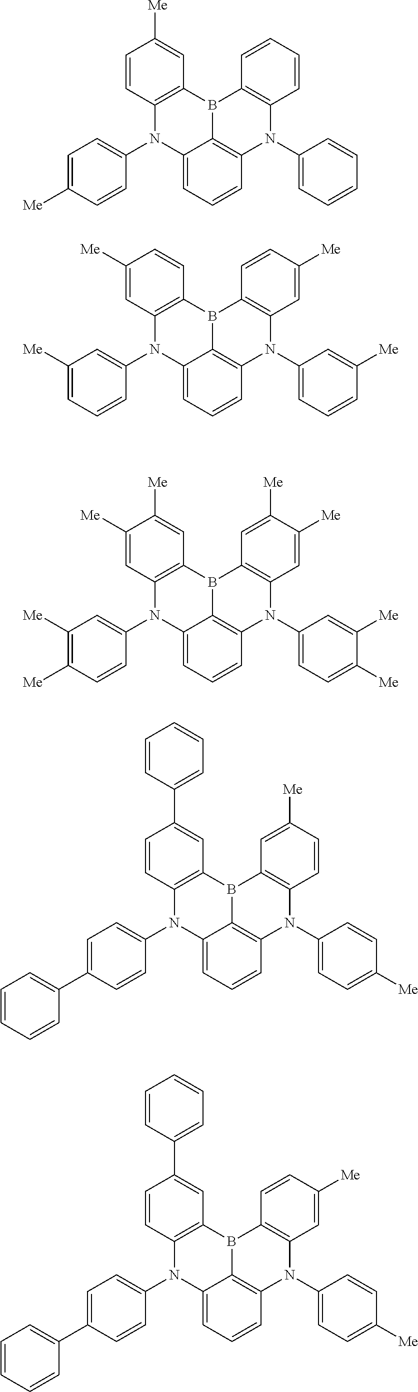

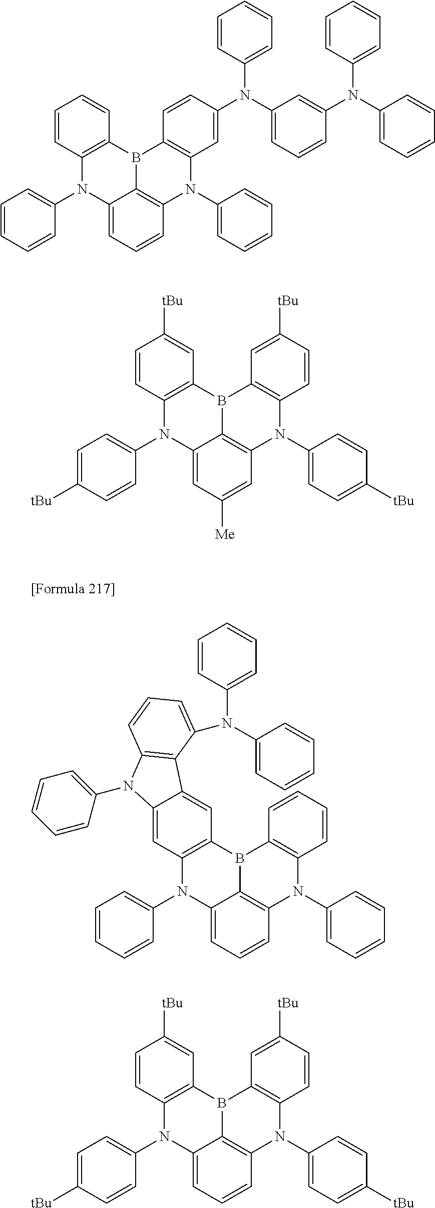 [SMILES: c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1]

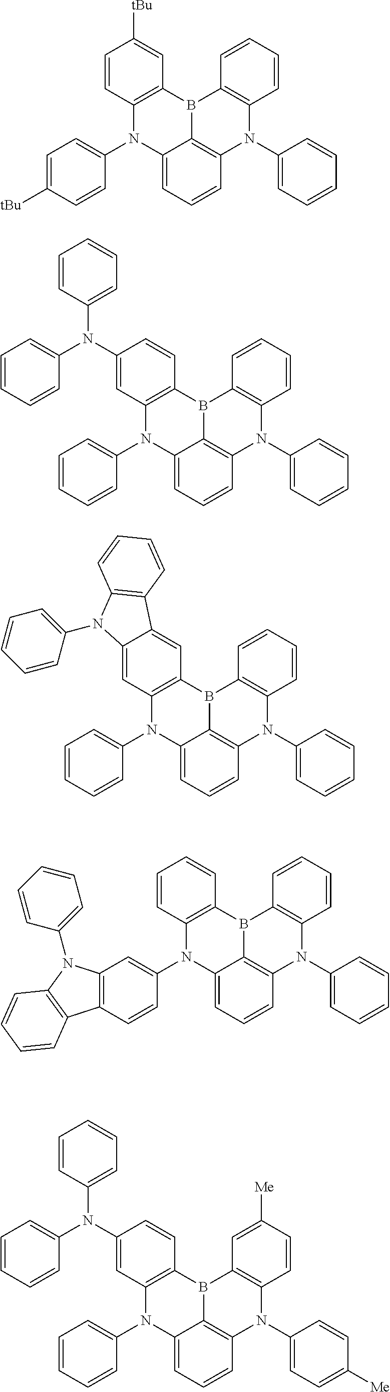

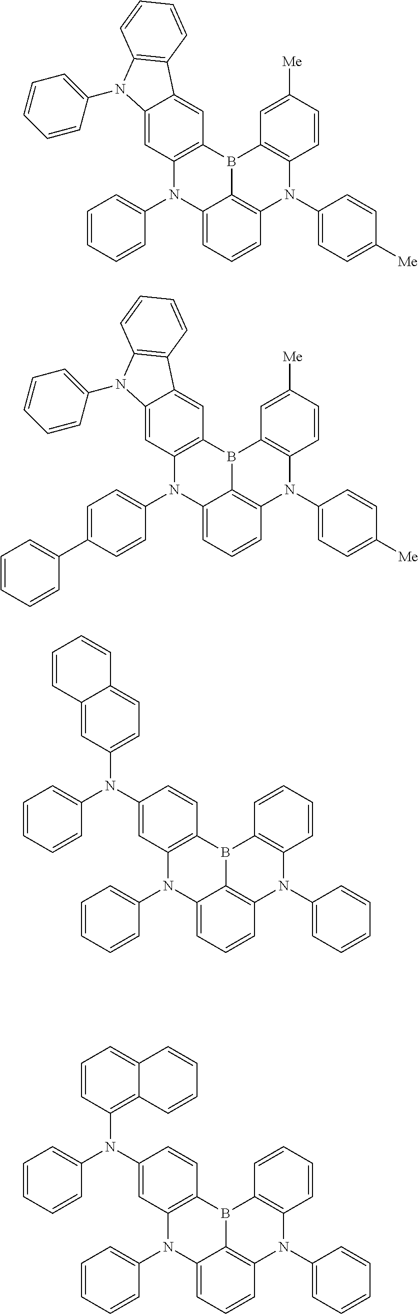

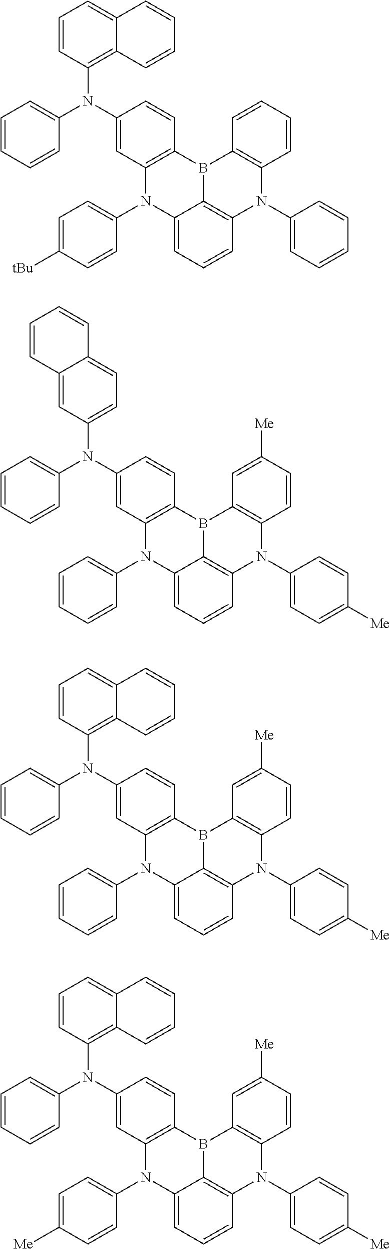

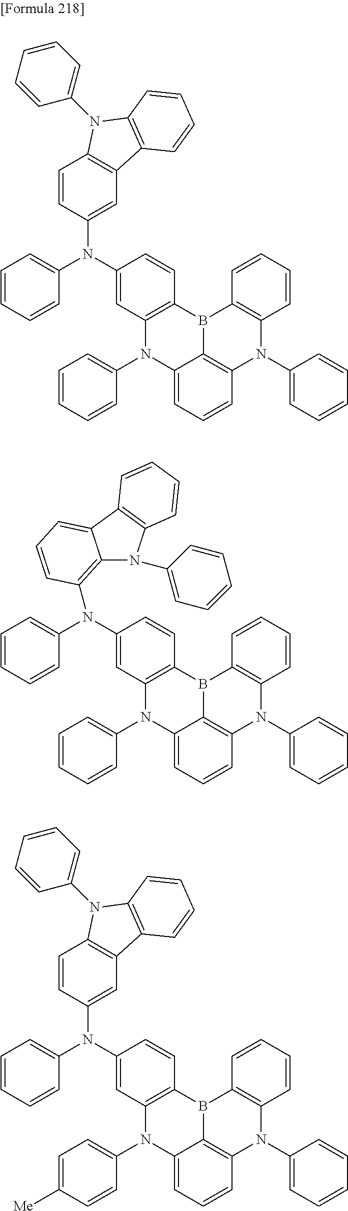 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

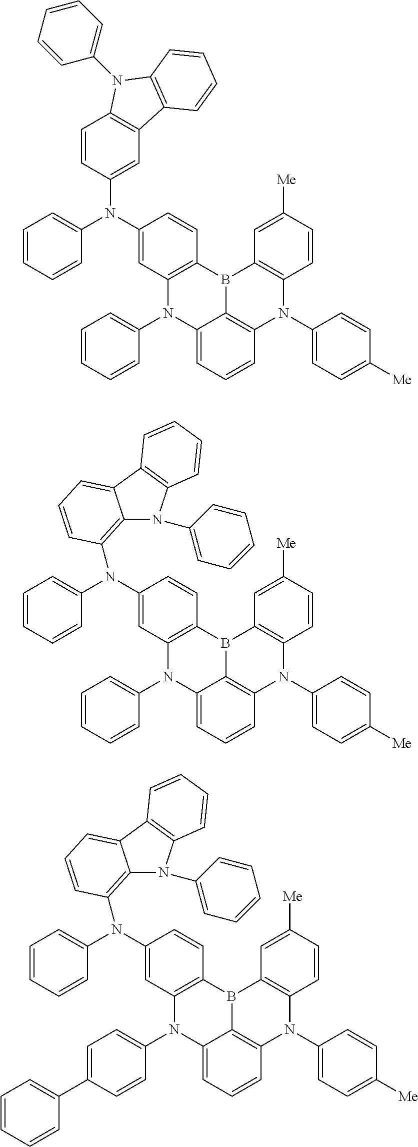 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

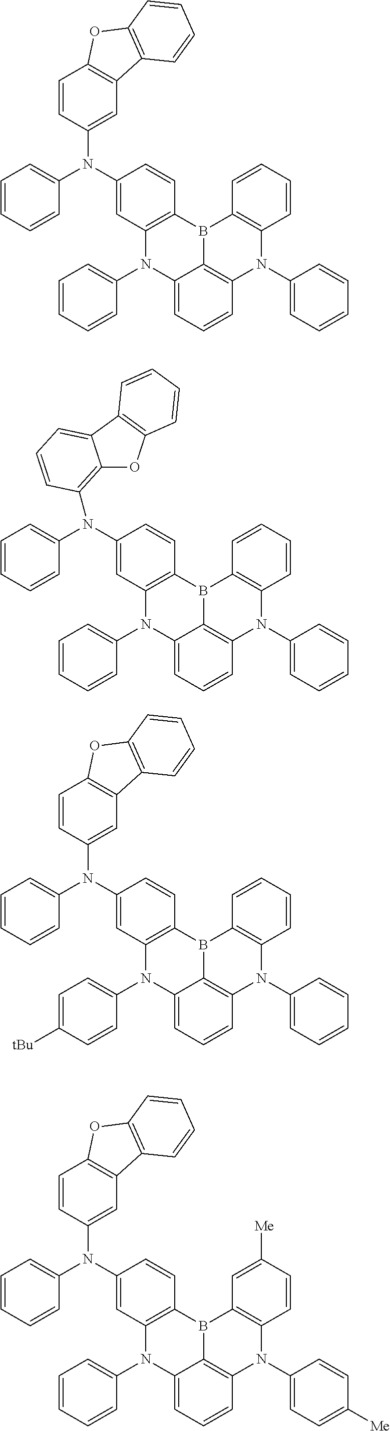

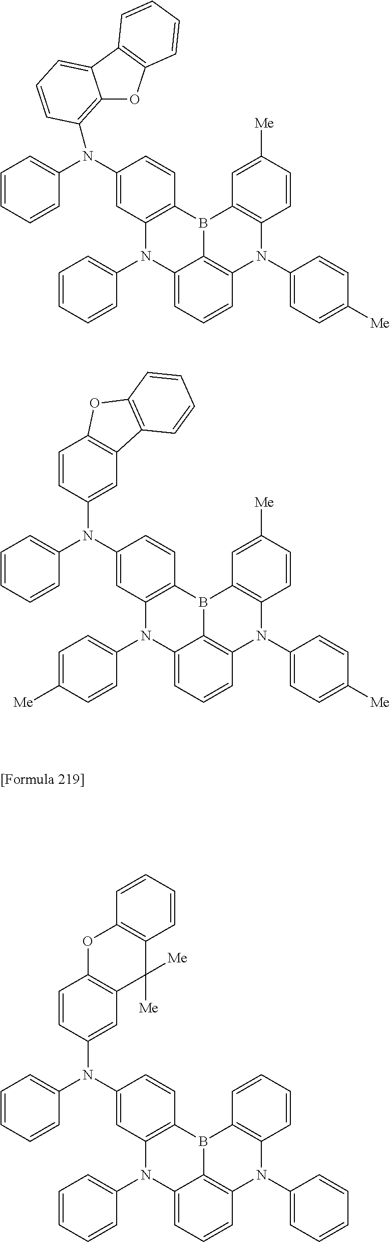 [SMILES: Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1 ; CC1(C)c2ccccc2Oc2ccc(N(c3ccccc3)c3ccc4c(c3)N(c3ccccc3)c3cccc5c3B4c3ccccc3N5c3ccccc3)cc21]

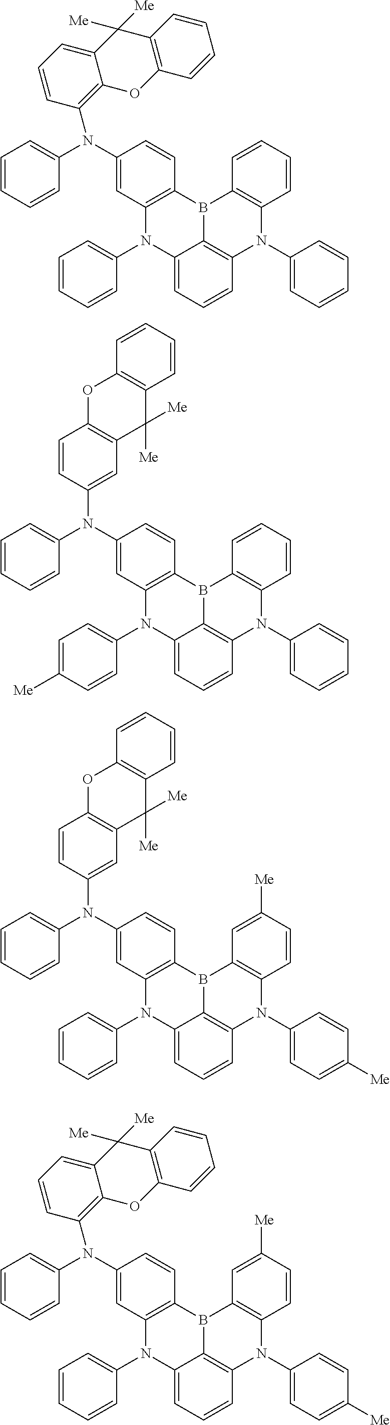

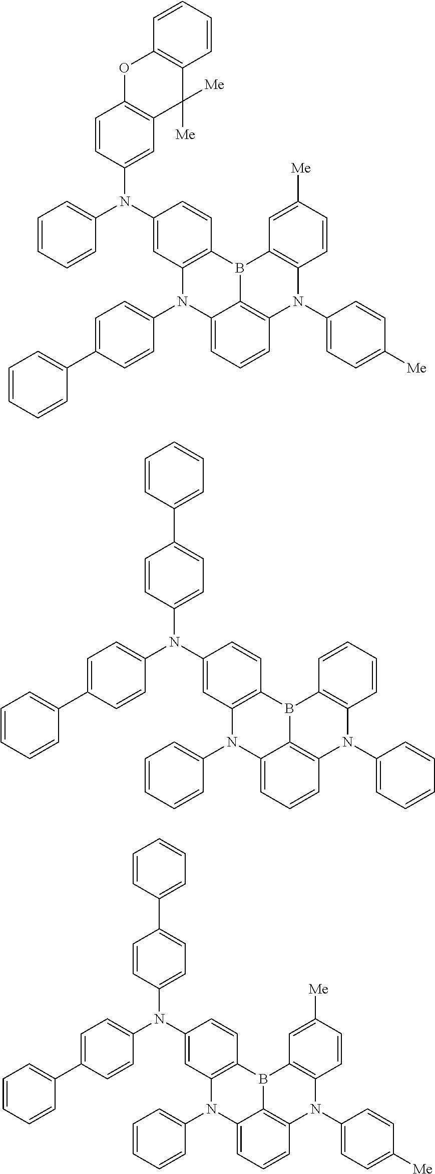

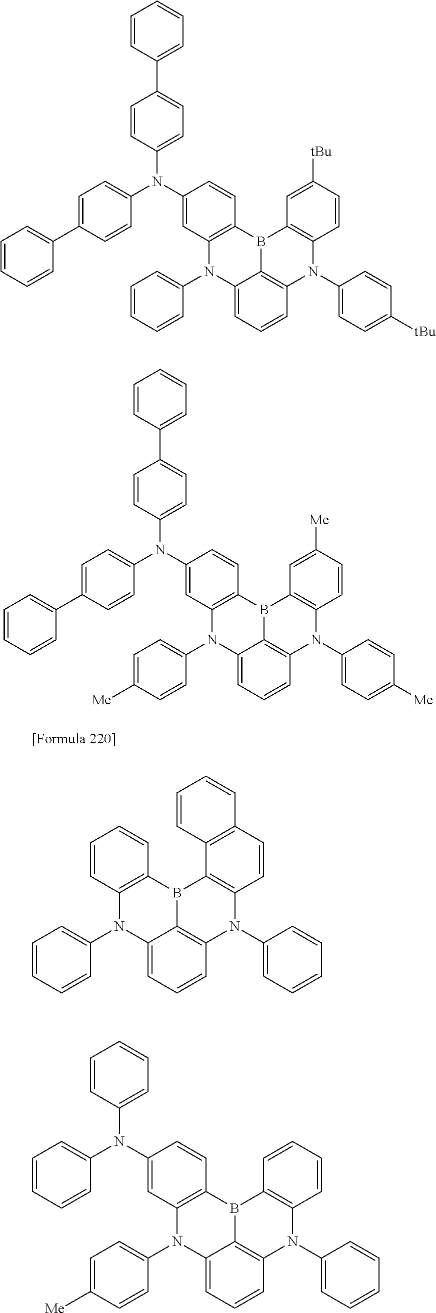

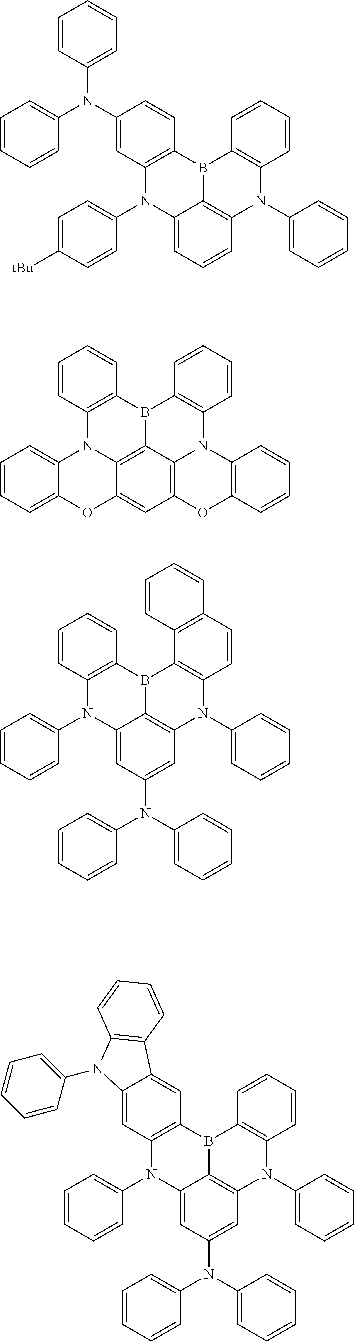 [SMILES: CC(C)(C)c1ccc(N2c3cc(N(c4ccccc4)c4ccccc4)ccc3B3c4ccccc4N(c4ccccc4)c4cccc2c43)cc1 ; c1ccc2c(c1)B1c3ccccc3-n3c4ccccc4oc4cc5oc6ccccc6n-2c5c1c43 ; c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1]

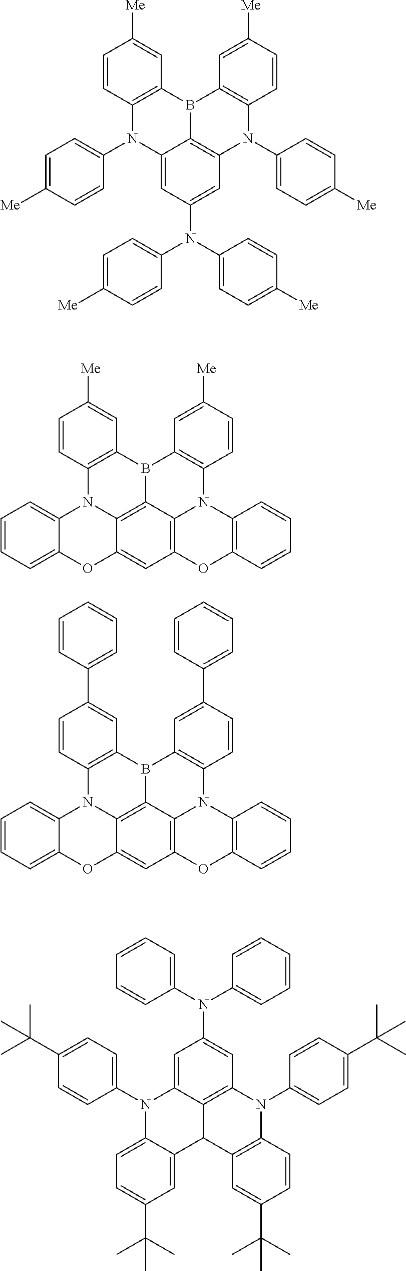

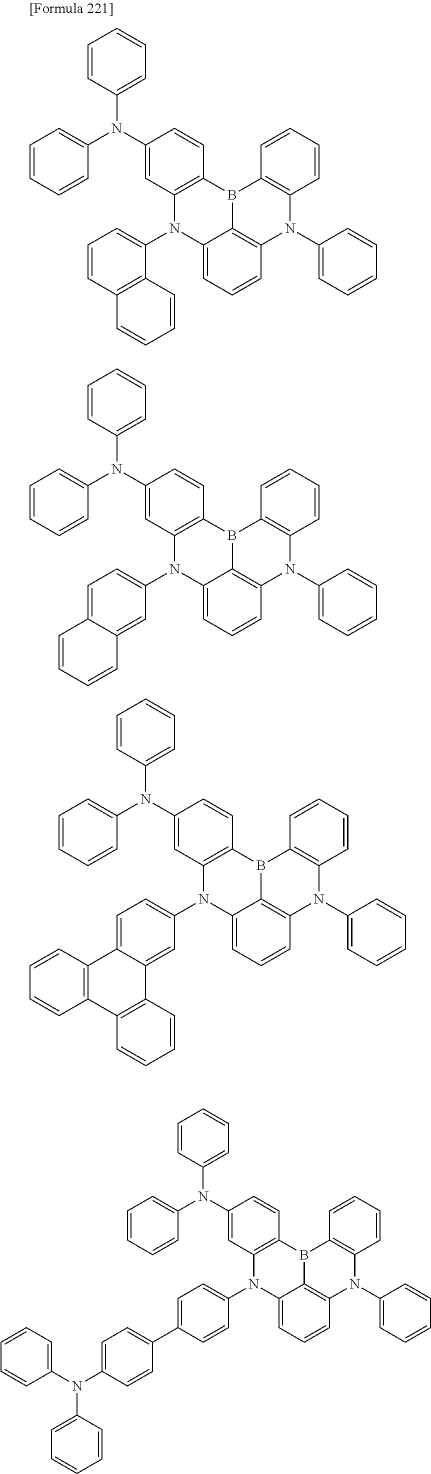

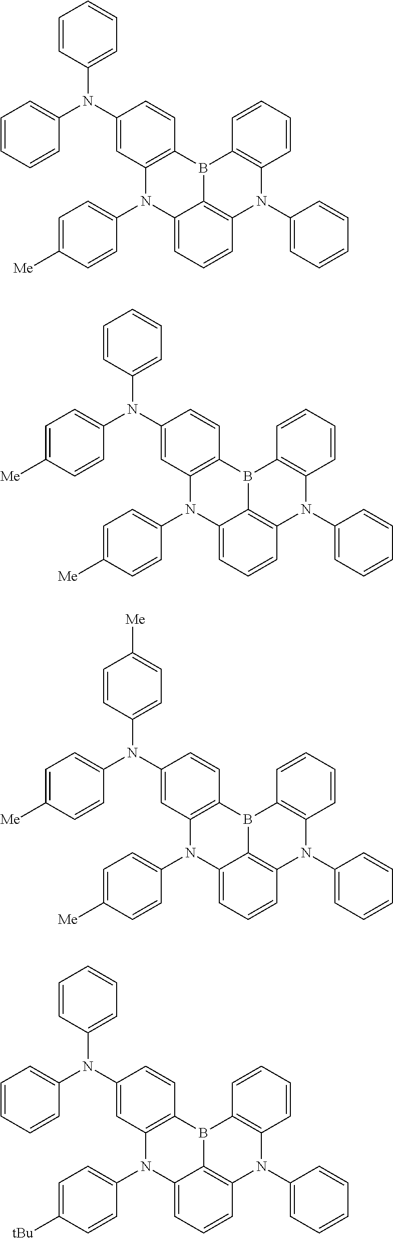

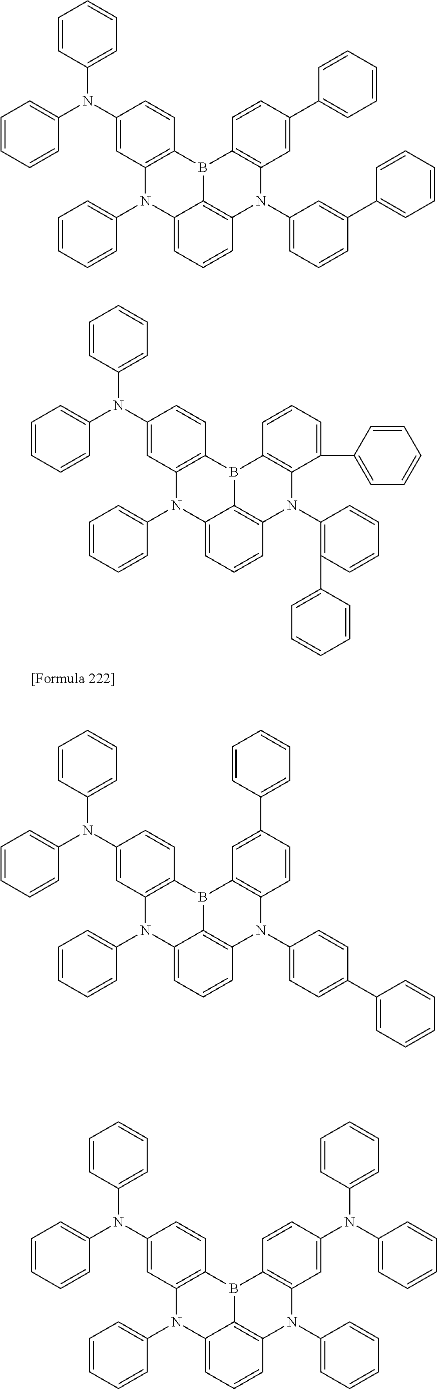 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1]

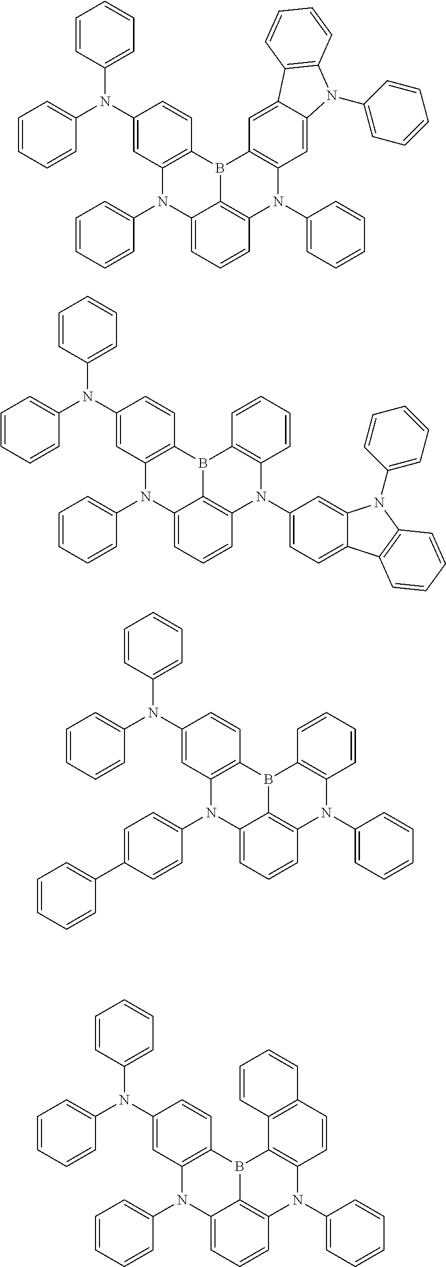

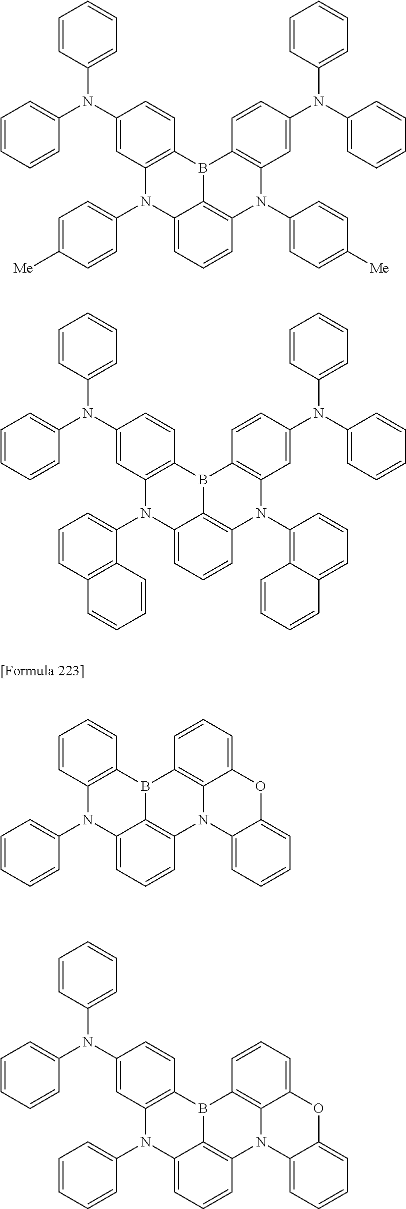

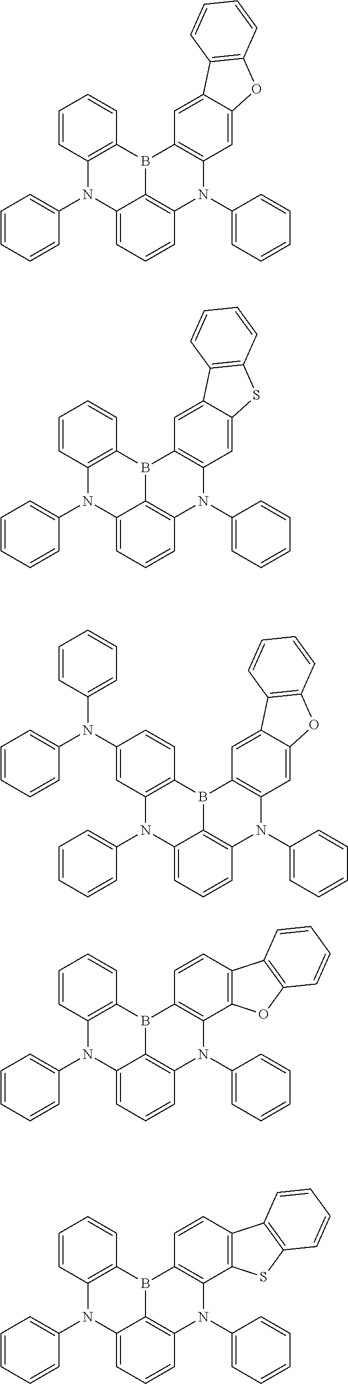

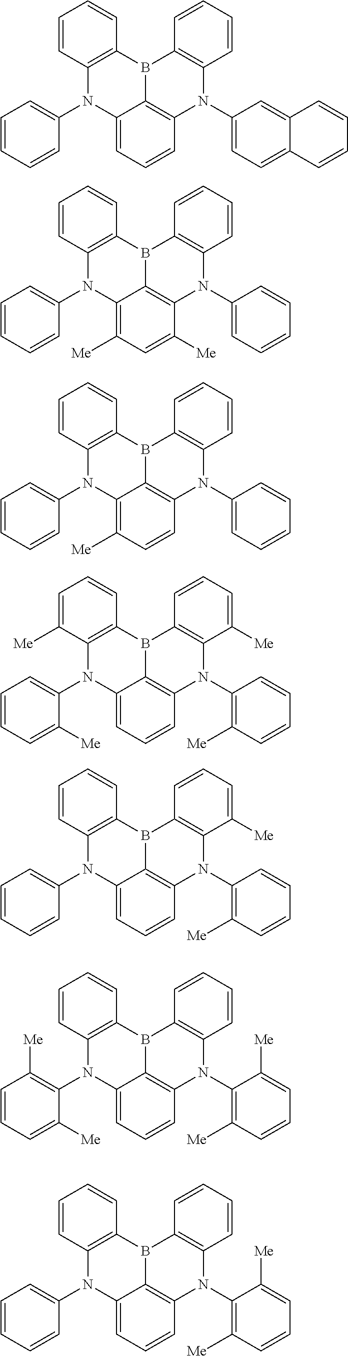

### Compound Represented by Formula (7)

The compound represented by the formula (7) will be described below.

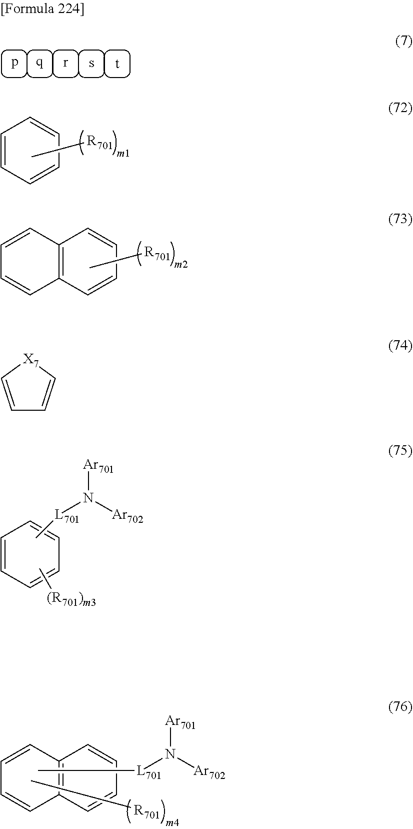

In the formula (7):

r ring is a ring represented by the formula (72) or the formula (73), the r ring being fused with any position(s) of adjacent ring(s);

q ring and s ring are each independently a ring represented by the formula (74) and fused with any position(s) of adjacent ring(s);

p ring and t ring are each independently a moiety represented by the formula (75) or the formula (76) and fused with any position(s) of adjacent ring(s);

X₇ is an oxygen atom, a sulfur atom, or NR₇₀₂;

when a plurality of R₇₀₁ are present, adjacent ones of the plurality of R₇₀₁ are mutually bonded to form a substituted or unsubstituted monocyclic ring, mutually bonded to form a substituted or unsubstituted fused ring, or not mutually bonded;

R₇₀₁ and R₇₀₂ not forming the monocyclic ring and not forming the fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;

Ar₇₀₁ and Ar₇₀₂ are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

L₇₀₁ is a substituted or unsubstituted alkylene group having 1 to 50 carbon atoms, a substituted or unsubstituted alkenylene group having 2 to 50 carbon atoms, a substituted or unsubstituted alkynylene group having 2 to 50 carbon atoms, a substituted or unsubstituted cycloalkylene group having 3 to 50 ring carbon atoms, a substituted or unsubstituted arylene group having 6 to 50 ring carbon atoms, or a substituted or unsubstituted divalent heterocyclic group having 5 to 50 ring atoms;

m1 is 0, 1, or 2;

m2 is 0, 1, 2, 3, or 4;

m3 is each independently 0, 1, 2, 3 or 3;

m4 is each independently 0, 1, 2, 3, 4, or 5;

when a plurality of R₇₀₁ are present, the plurality of R₇₀₁ are mutually the same or different;

when a plurality of X₇ are present, the plurality of X₇ are mutually the same or different;

when a plurality of R₇₀₂ are present, the plurality of R₇₀₂ are mutually the same or different;

when a plurality of Ar₇₀₁ are present, the plurality of Ar₇₀₁ are mutually the same or different;

when a plurality of Ar₇₀₂ are present, the plurality of Ar₇₀₂ are mutually the same or different; and

when a plurality of L₇₀₁ are present, the plurality of L₇₀₁ are mutually the same or different.

In the formula (7), each of the p ring, q ring, r ring, s ring, and t ring is fused with an adjacent ring(s) sharing two carbon atoms. The fused position and orientation are not limited but may be defined as required.

In an exemplary embodiment, in the formula (72) or the formula (73) representing the r ring, m1=0 or m2=0 is satisfied.

In an exemplary embodiment, the compound represented by the formula (7) is represented by any one of formulae (71-1) to (71-6) below.

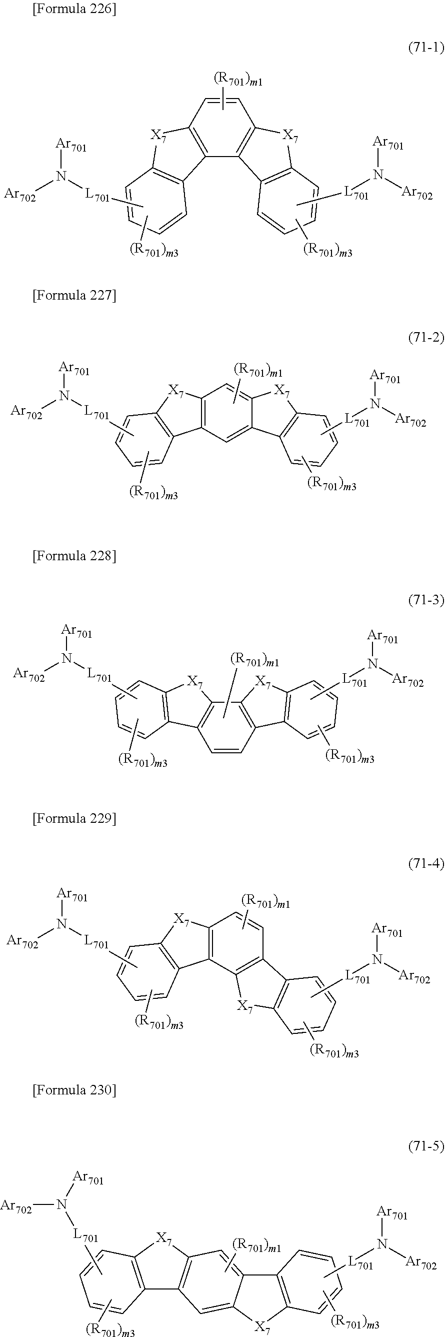

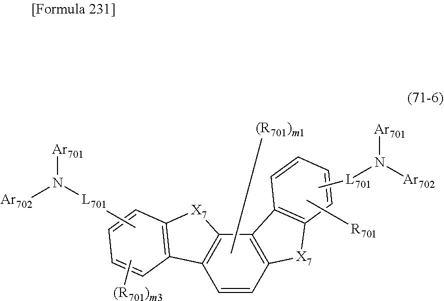

In the formulae (71-1) to (71-6), R₇₀₁, X₇, Ar₇₀₁, Ar₇₀₂, L₇₀₁, m1 and m3 respectively represent the same as R₇₀₁, X₇, Ar₇₀₁, Ar₇₀₂, L₇₀₁, m1 and m3 in the formula (7).

In an exemplary embodiment, the compound represented by the formula (7) is represented by any one of formulae (71-11) to (71-13) below.

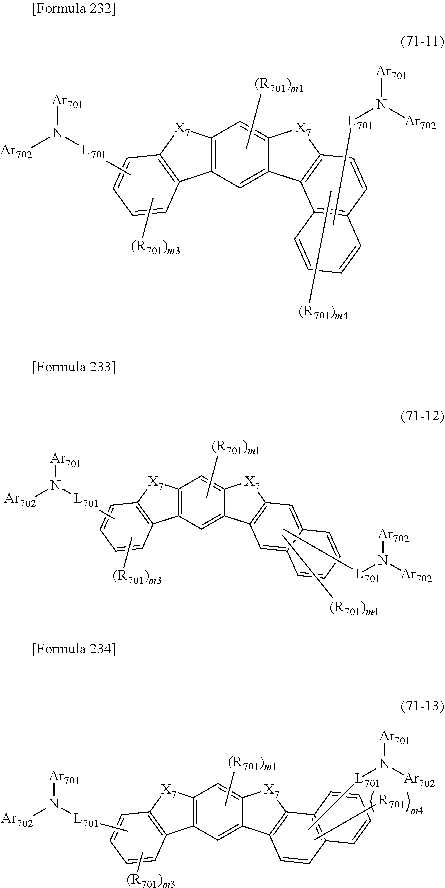

In the formulae (71-11) to (71-13), R₇₀₁, X₇, Ar₇₀₁, Ar₇₀₂, L₇₁₁, m1, m3 and m4 respectively represent the same as R₇₀₁, X₇, Ar₇₀₁, Ar₇₀₂, L₇₁₁, m1, m3 and m4 in the formula (7).

In an exemplary embodiment, the compound represented by the formula (7) is represented by any one of formulae (71-21) to (71-25) below.

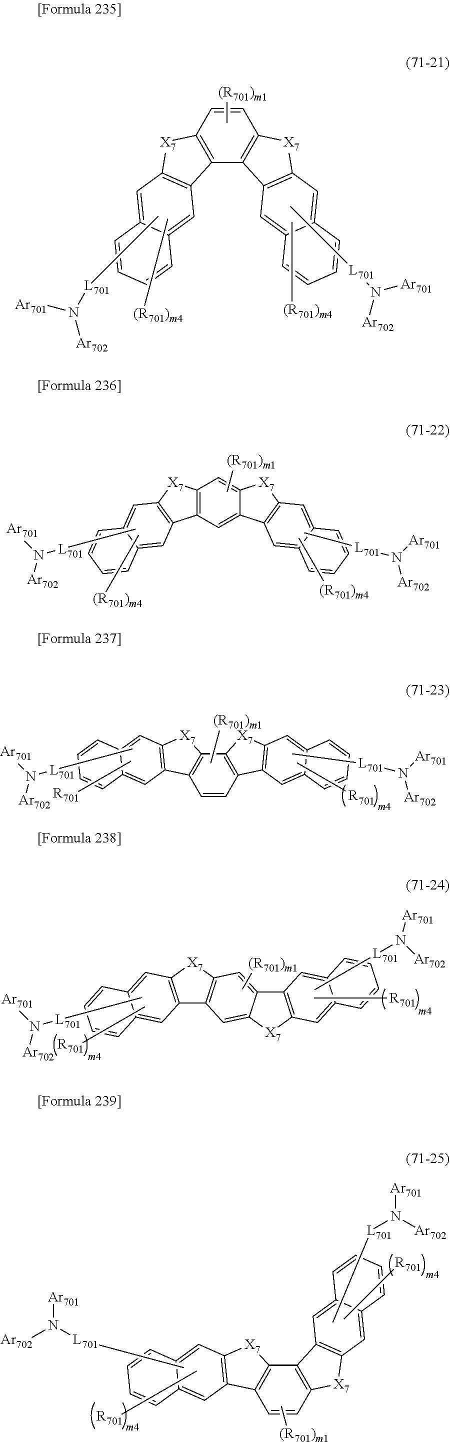

In the formulae (71-21) to (71-25), R₇₀₁, X₇, Ar₇₀₁, Ar₇₀₂, L₇₀₁, m1 and m4 respectively represent the same as R₇₀₁, X₇, Ar₇₀₁, Ar₇₀₂, L₇₀₁, m1 and m4 in the formula (7).

In an exemplary embodiment, the compound represented by the formula (7) is represented by any one of formulae (71-31) to (71-33) below.

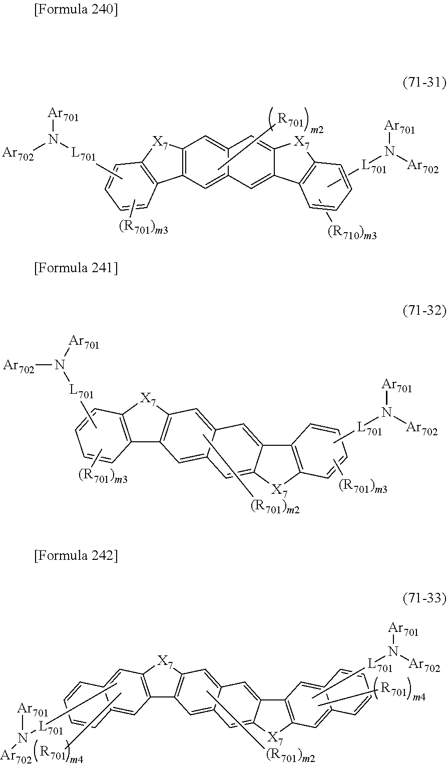

In the formulae (71-31) to (71-33), R₇₀₁, X₇, Ar₇₀₁, Ar₇₀₂, L₇₀₁, m2 to m4 respectively represent the same as R₇₀₁, X₇, Ar₇₀₁, Ar₇₀₂, L₇₀₁, m2 to m4 in the formula (7).

In an exemplary embodiment, Ar₇₀₁ and Ar₇₀₂ are each independently a substituted or unsubstituted aryl group having 6 to 50 ring carbon atoms.

In an exemplary embodiment, one of Ar₇₀₁ and Ar₇₀₂ is a substituted or unsubstituted aryl group having 6 to 50 ring carbon atoms, and the other of Ar₇₀₁ and Ar₇₀₂ is a substituted or unsubstituted heterocyclic group having 5 to 50 ring atoms.

Specific examples of the compound represented by the formula (7) include compounds shown below.

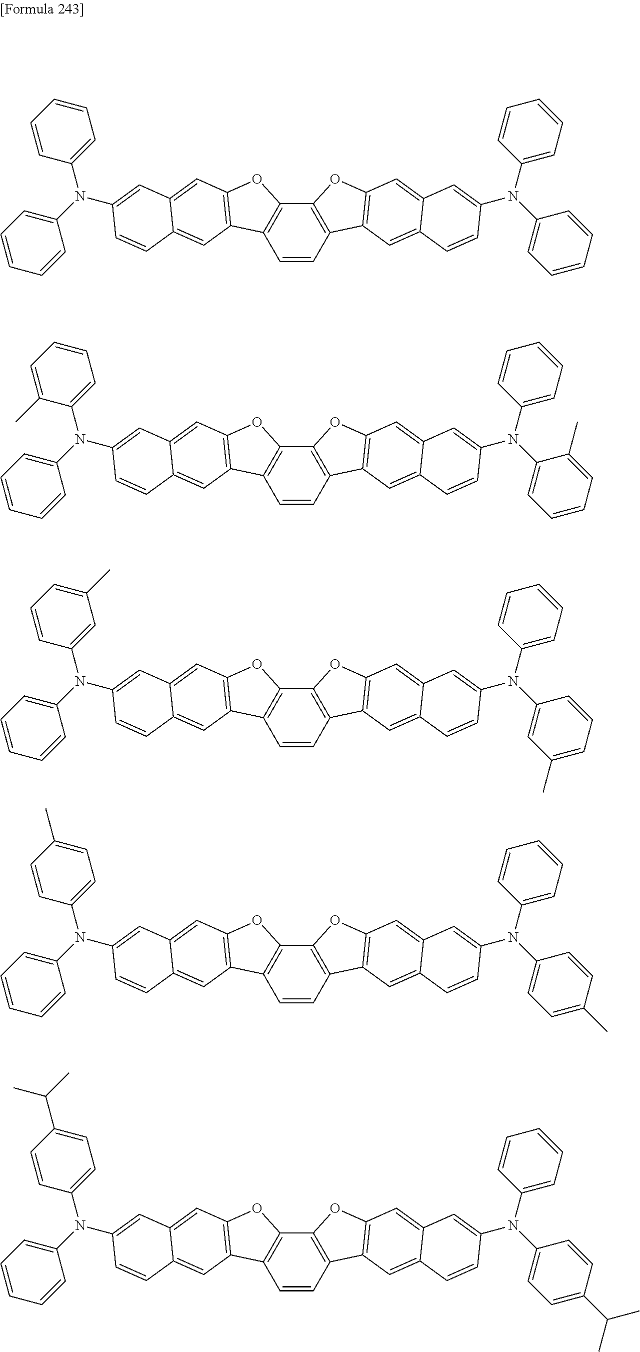 [SMILES: c1ccc(N(c2ccccc2)c2ccc3cc4c(cc3c2)oc2c4ccc3c4cc5ccc(N(c6ccccc6)c6ccccc6)cc5cc4oc32)cc1 ; Cc1ccccc1N(c1ccccc1)c1ccc2cc3c(cc2c1)oc1c3ccc2c3cc4ccc(N(c5ccccc5)c5ccccc5C)cc4cc3oc21 ; Cc1cccc(N(c2ccccc2)c2ccc3cc4c(cc3c2)oc2c4ccc3c4cc5ccc(N(c6ccccc6)c6cccc(C)c6)cc5cc4oc32)c1 ; Cc1ccc(N(c2ccccc2)c2ccc3cc4c(cc3c2)oc2c4ccc3c4cc5ccc(N(c6ccccc6)c6ccc(C)cc6)cc5cc4oc32)cc1 ; CC(C)c1ccc(N(c2ccccc2)c2ccc3cc4c(cc3c2)oc2c4ccc3c4cc5ccc(N(c6ccccc6)c6ccc(C(C)C)cc6)cc5cc4oc32)cc1]

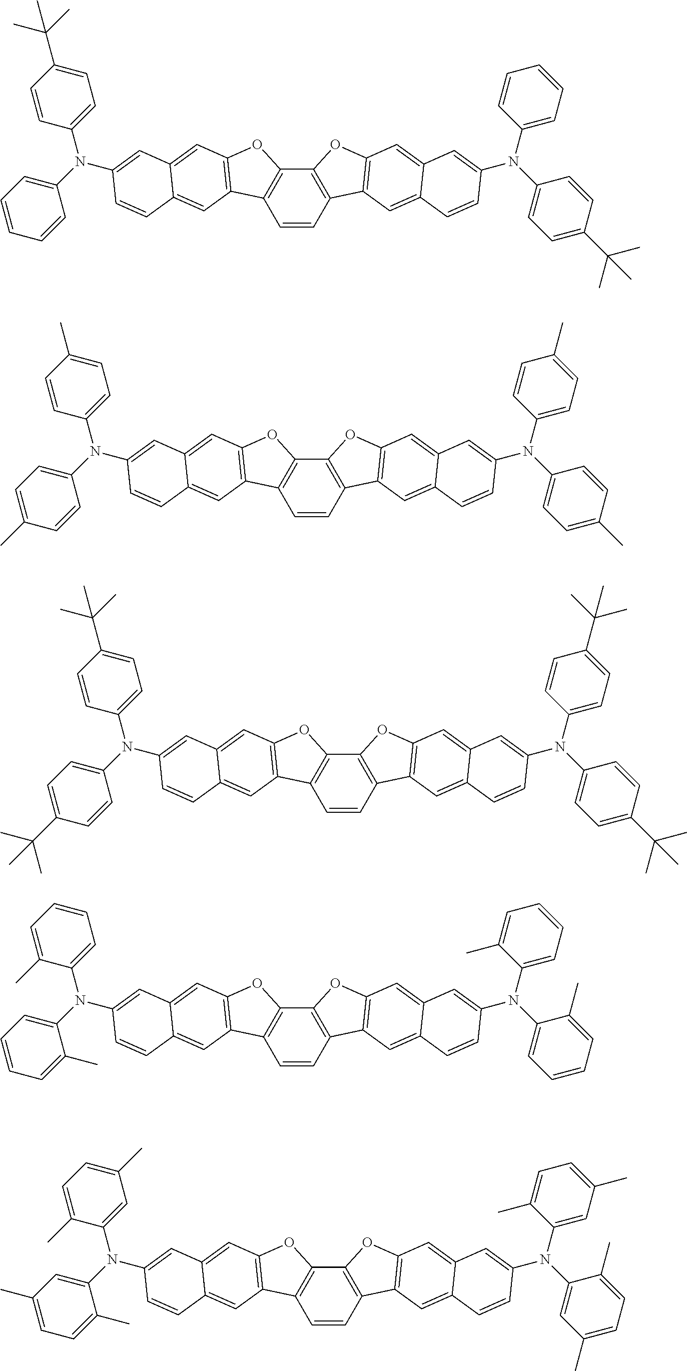

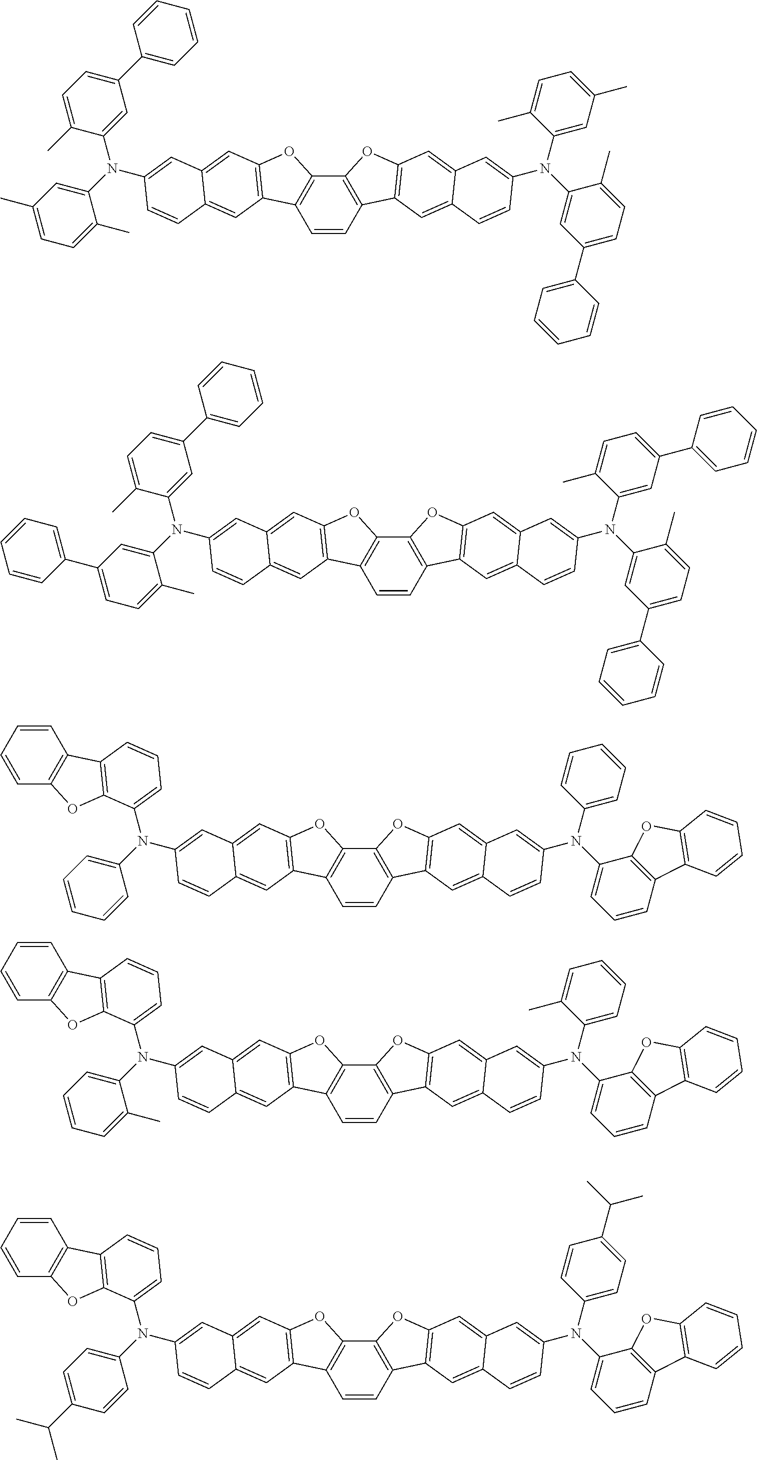

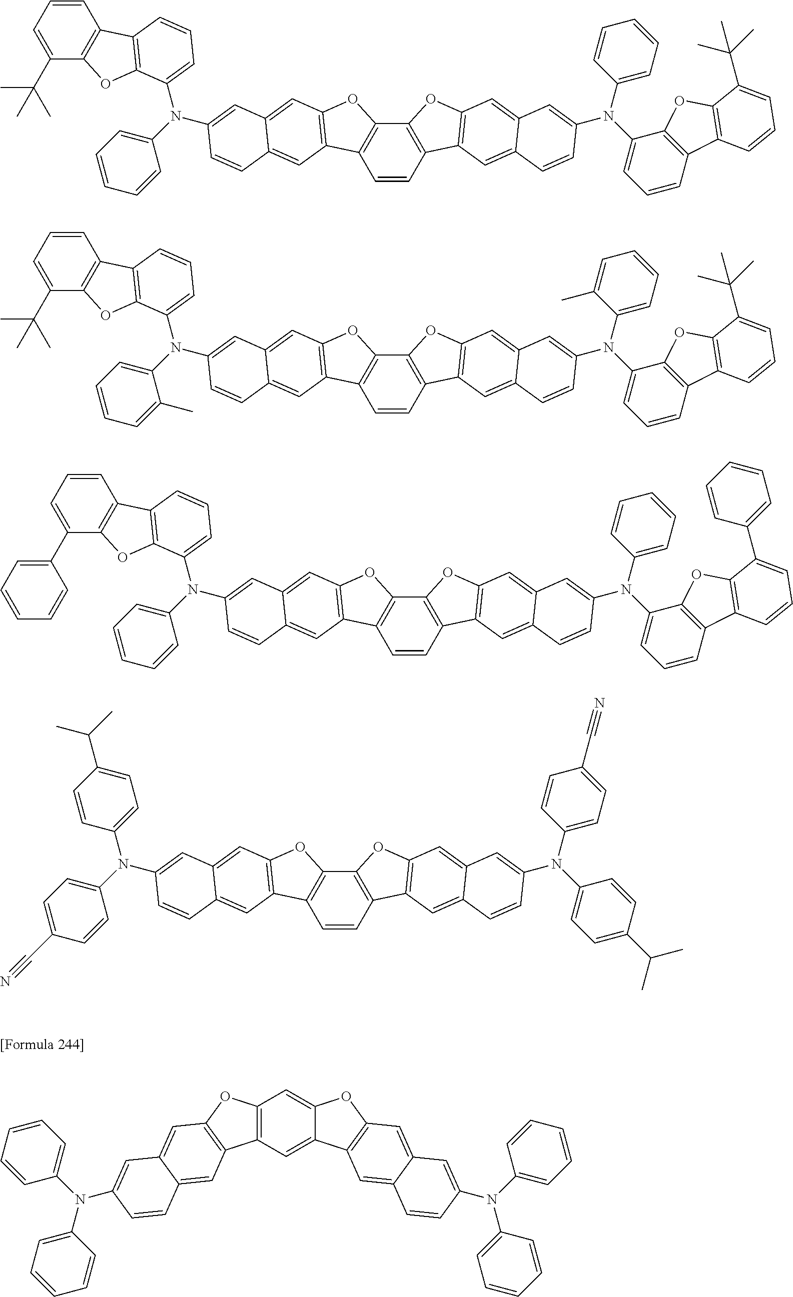

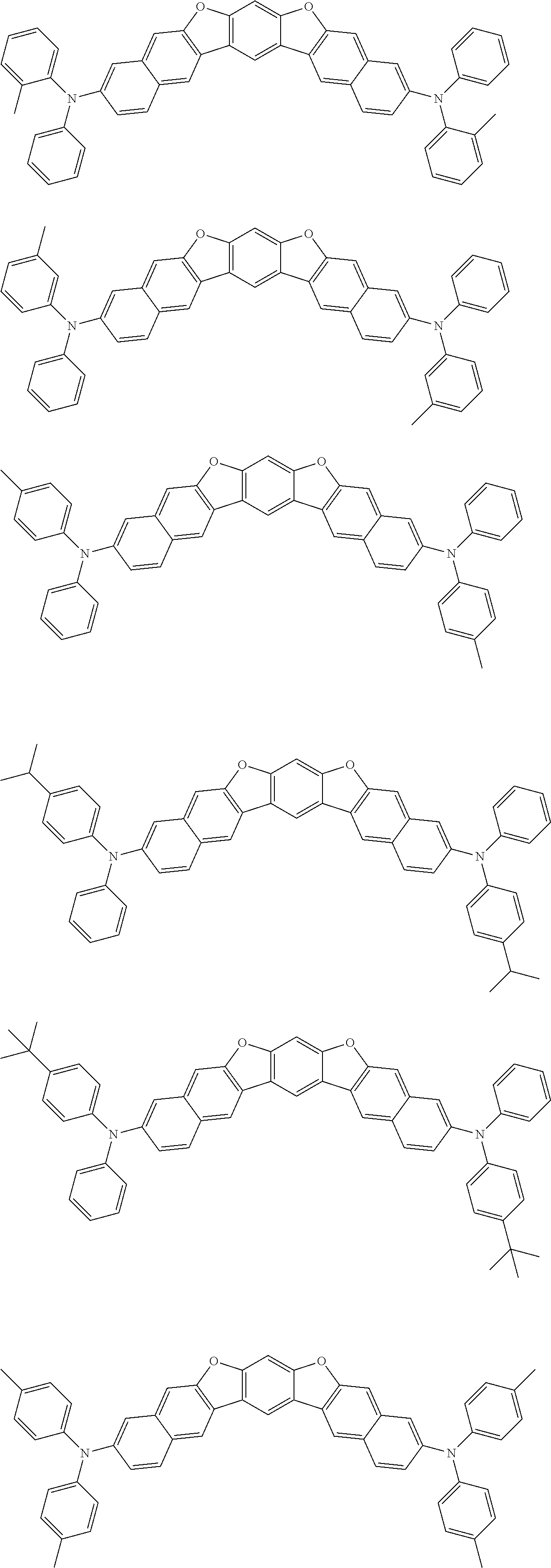

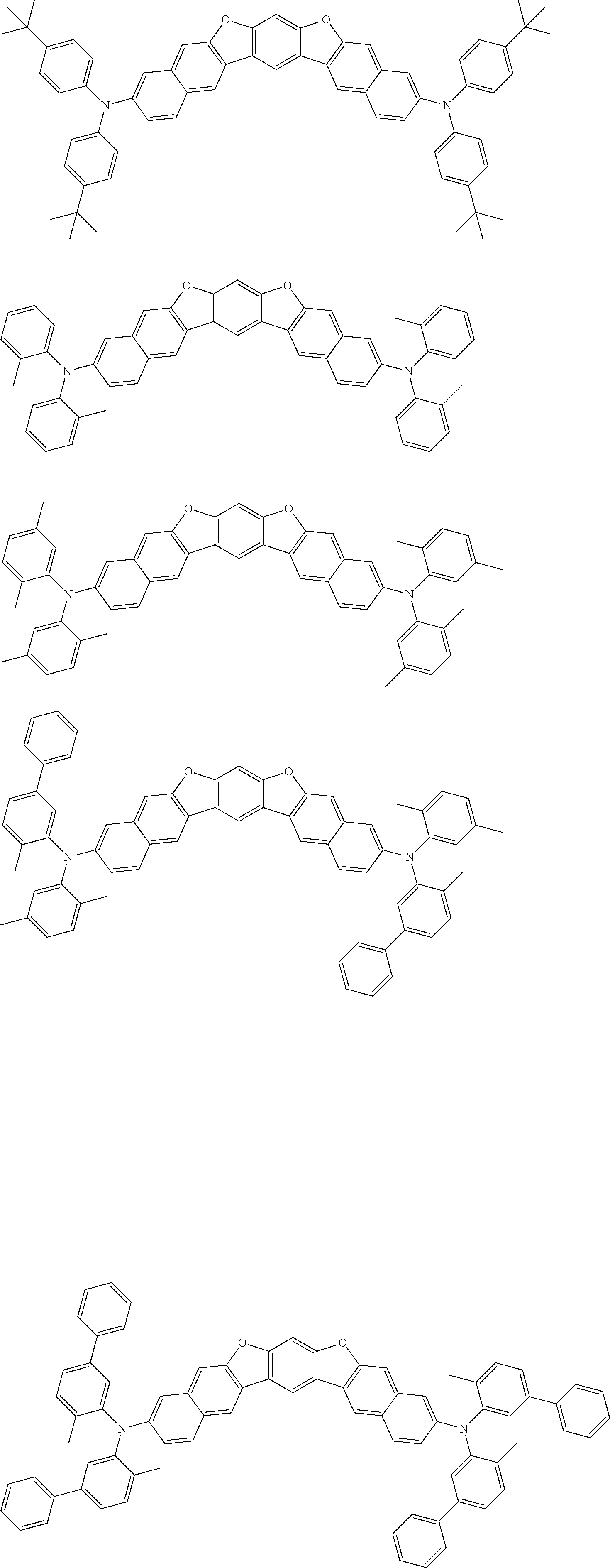

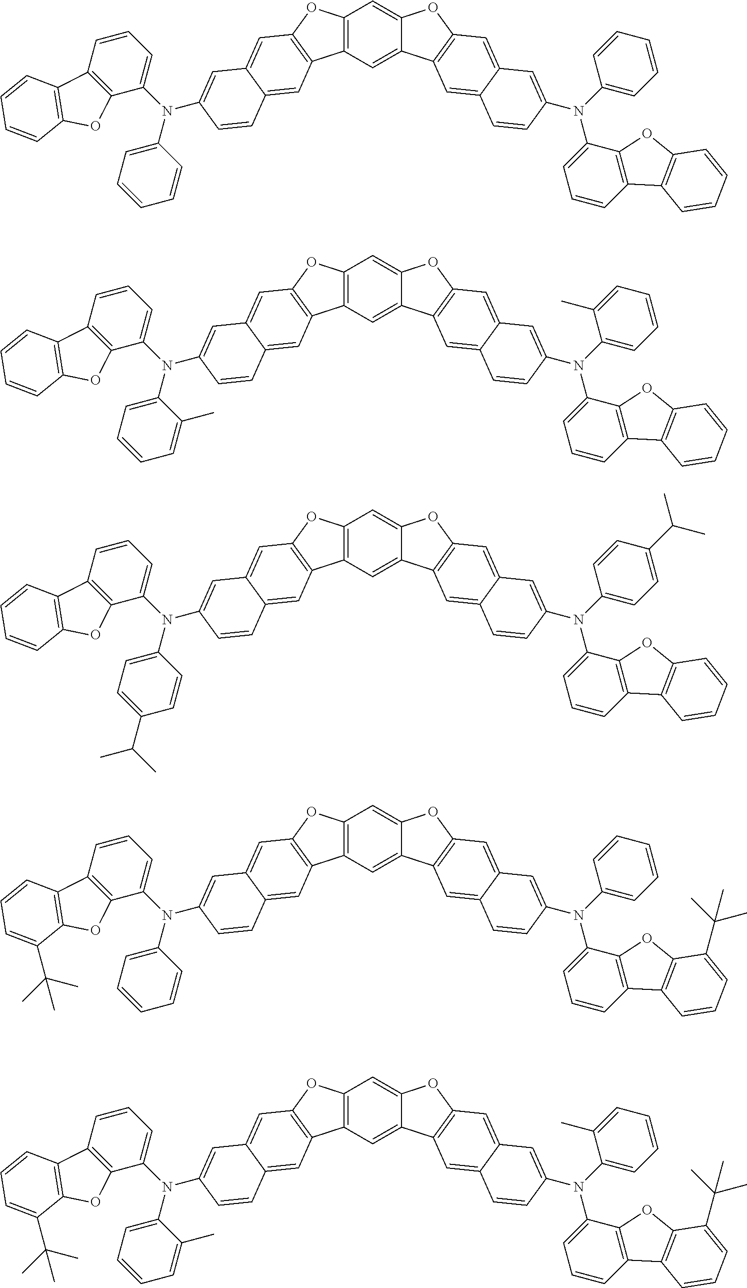

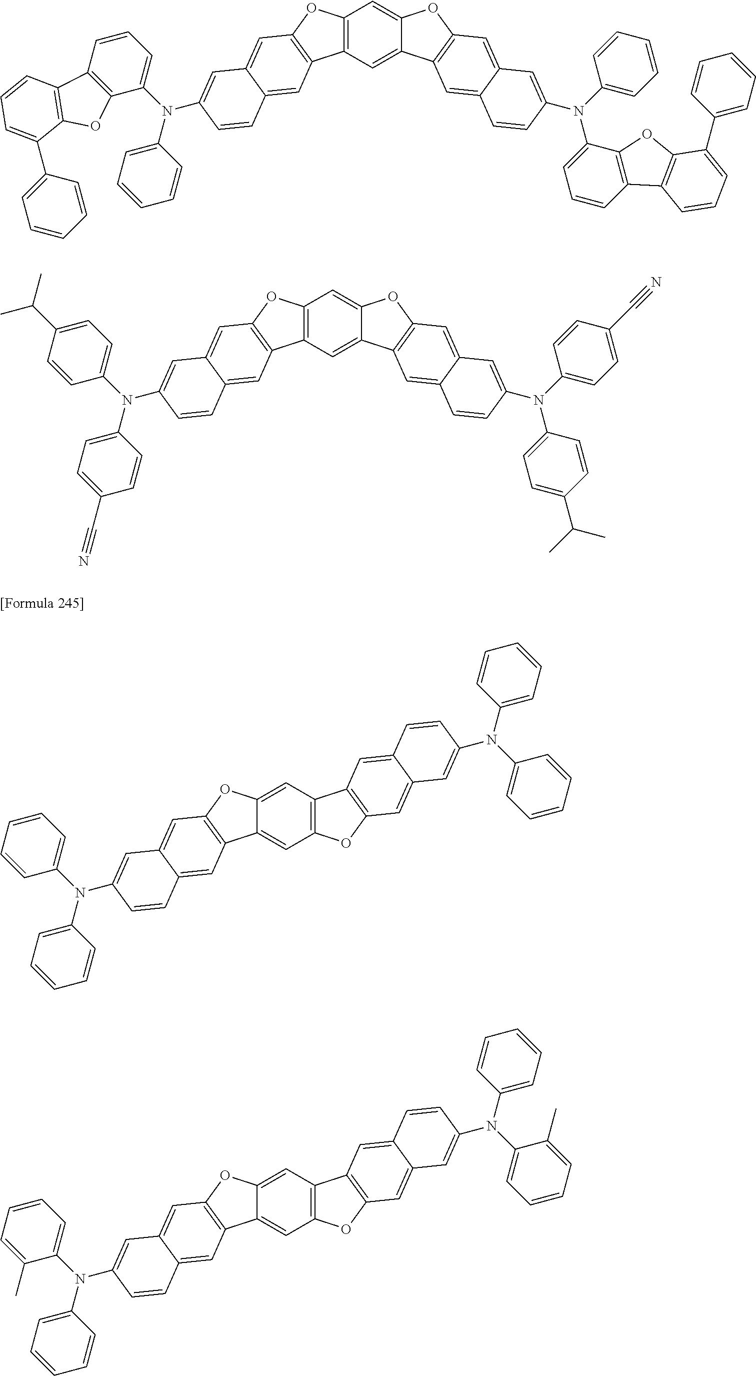

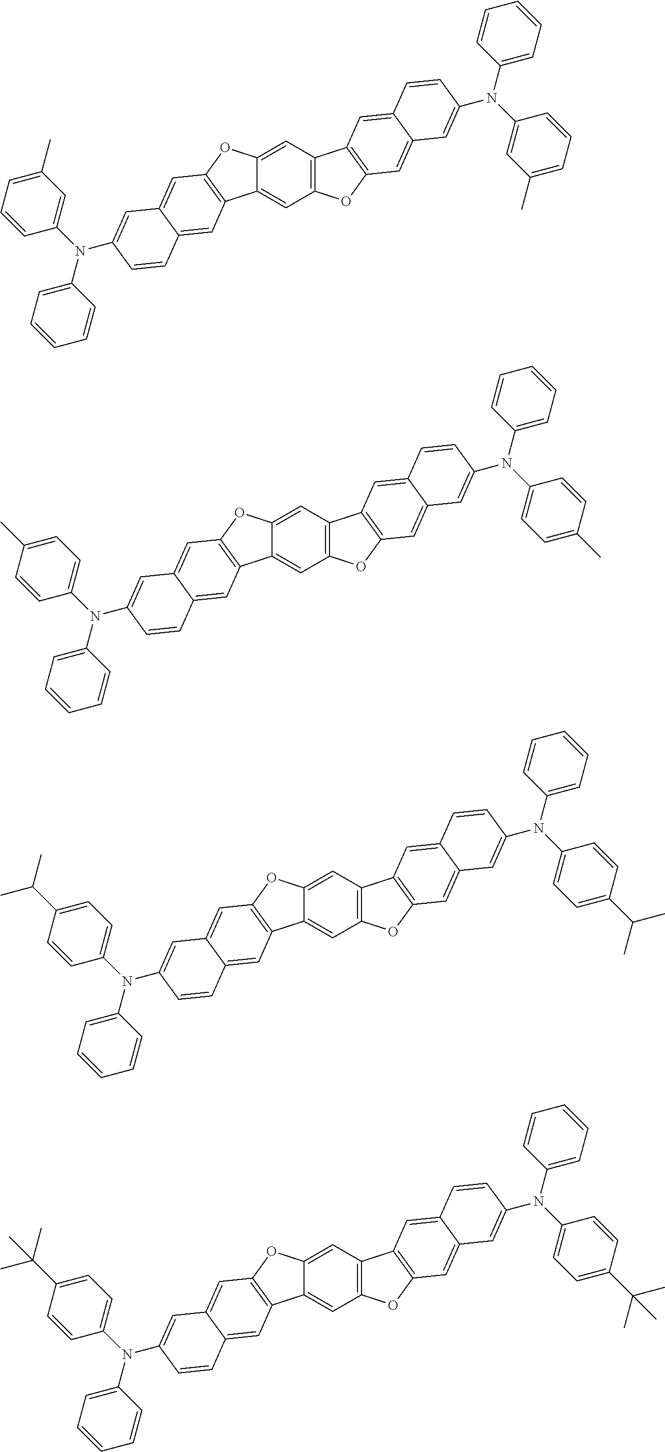

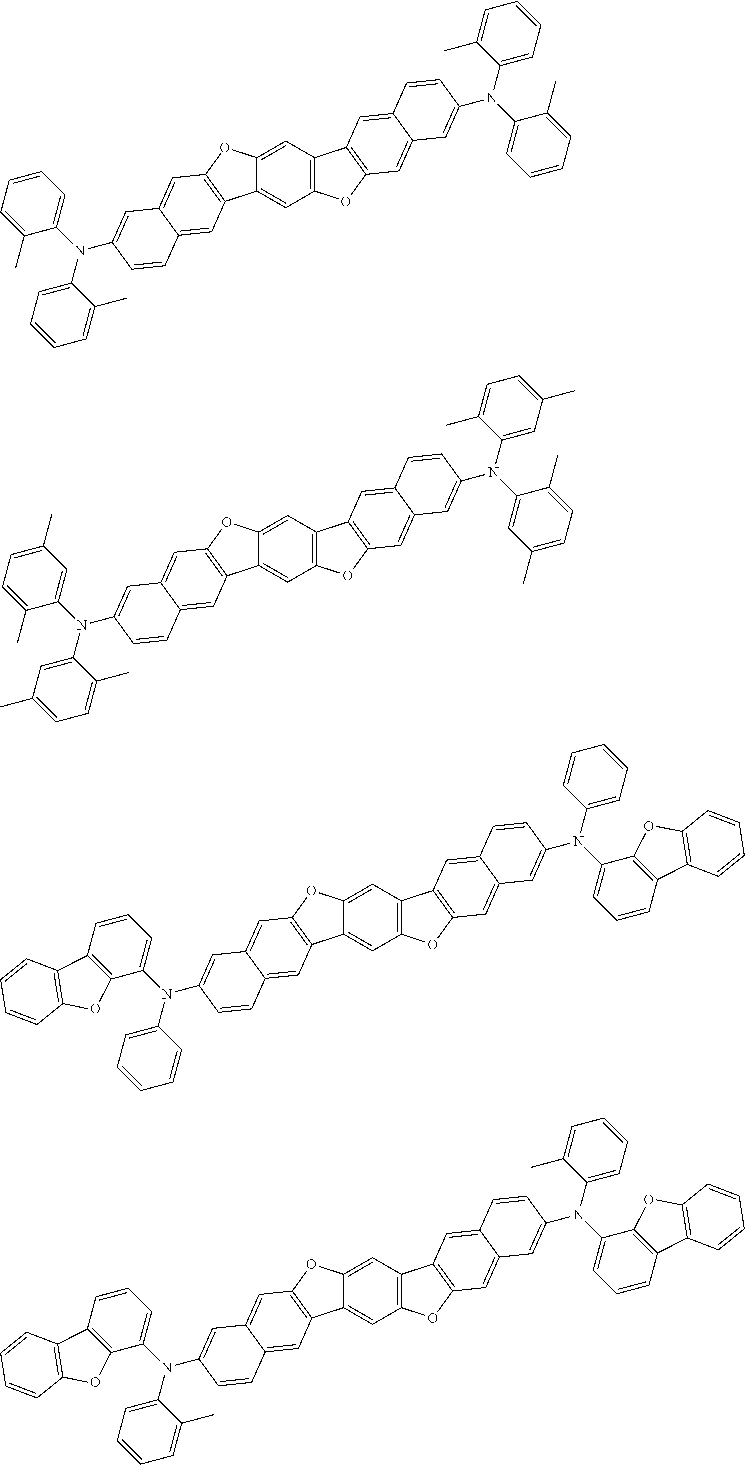

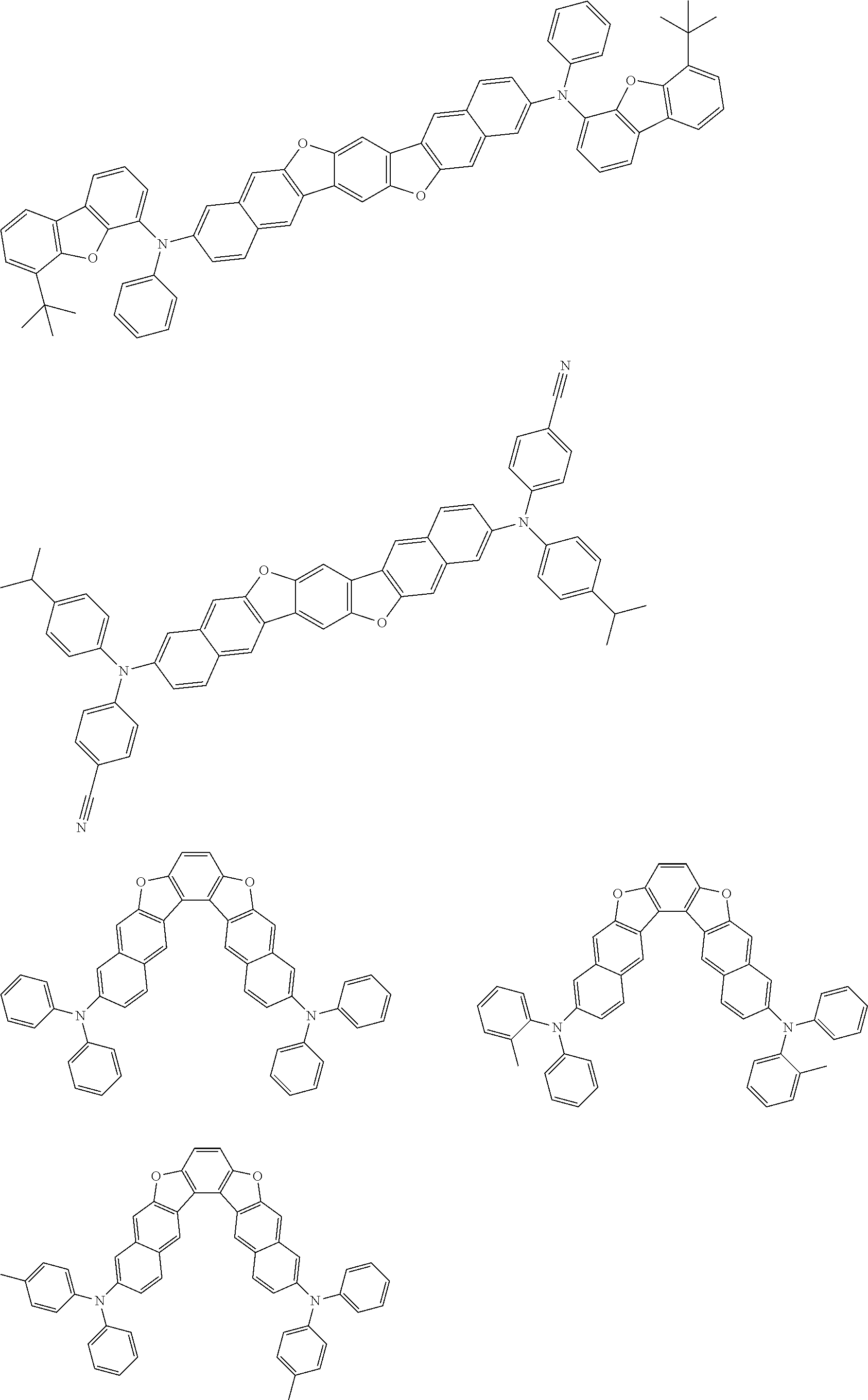

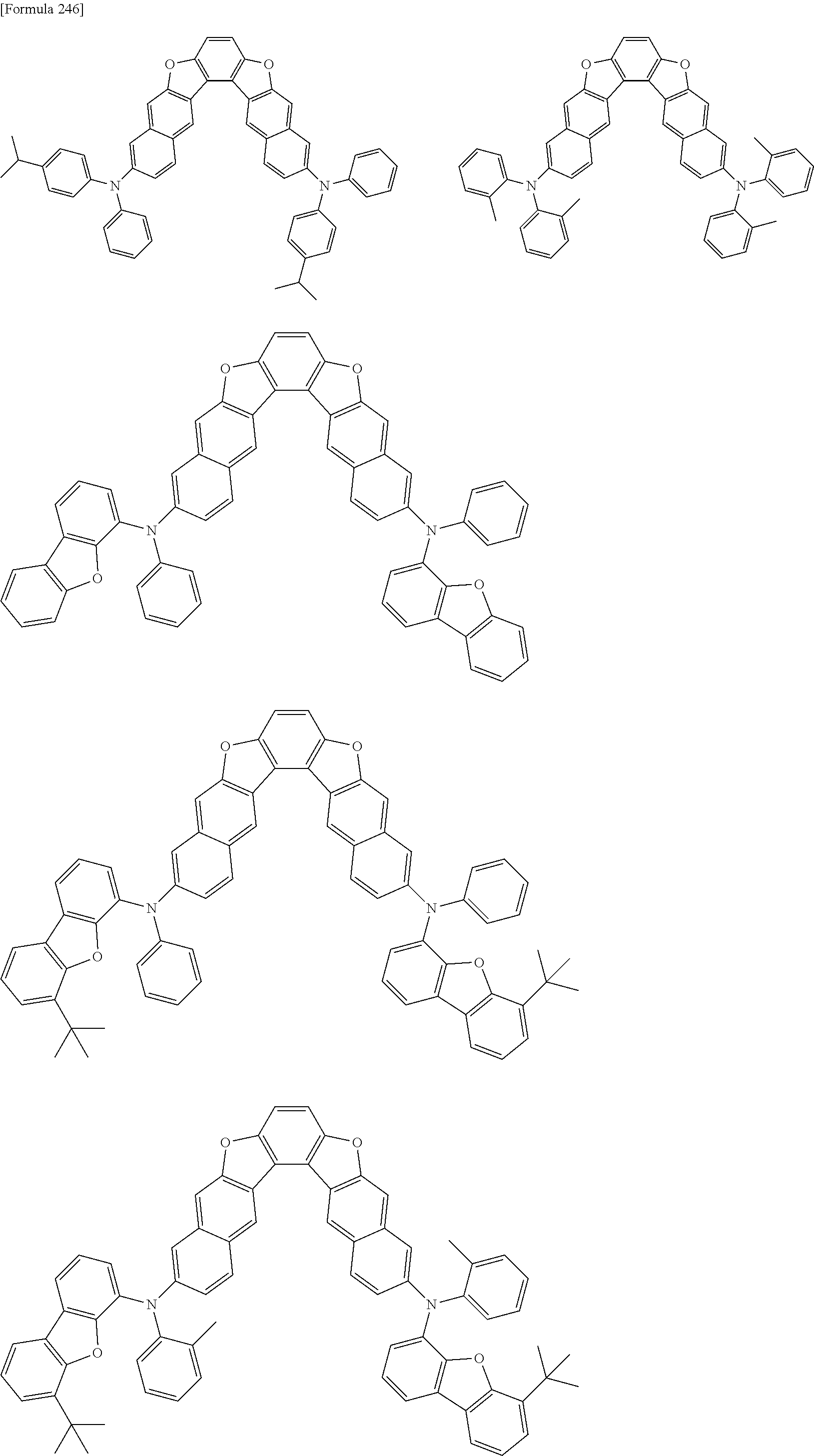

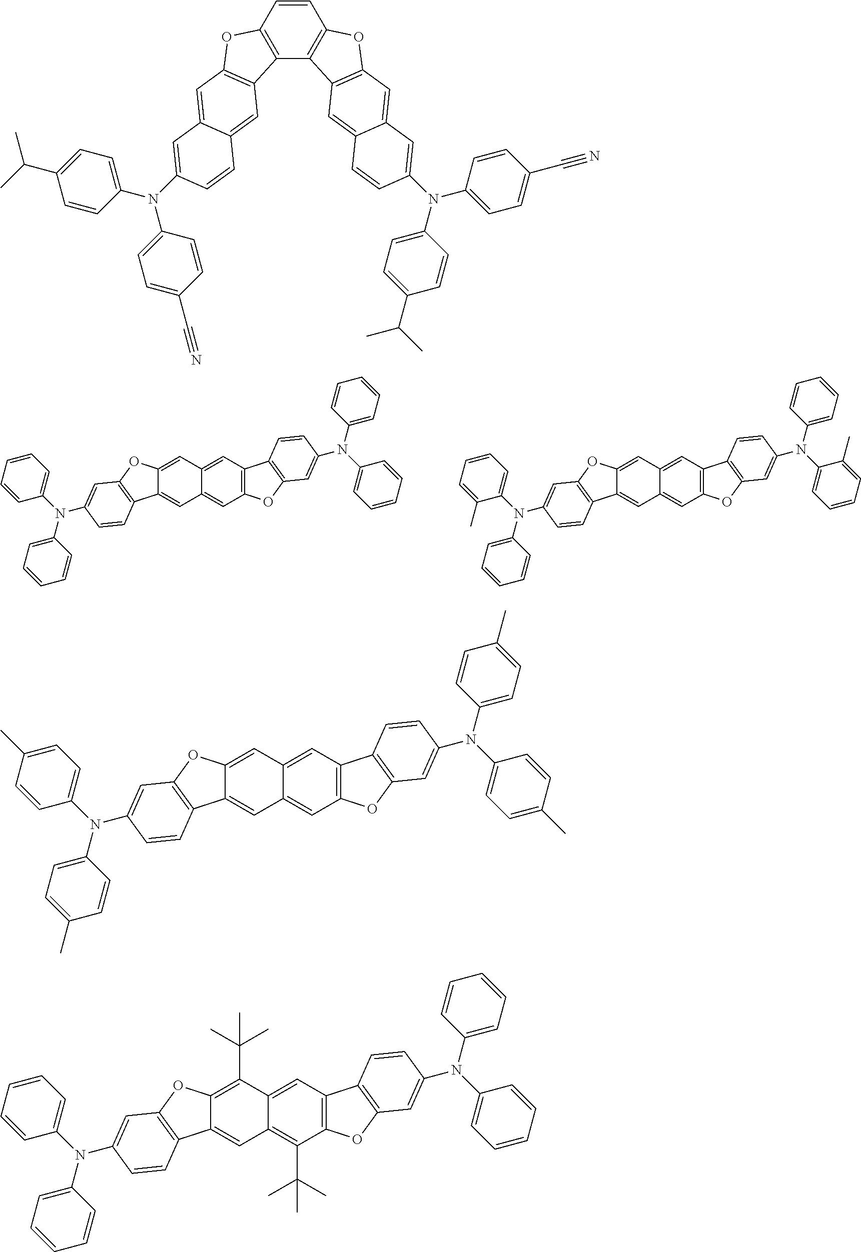

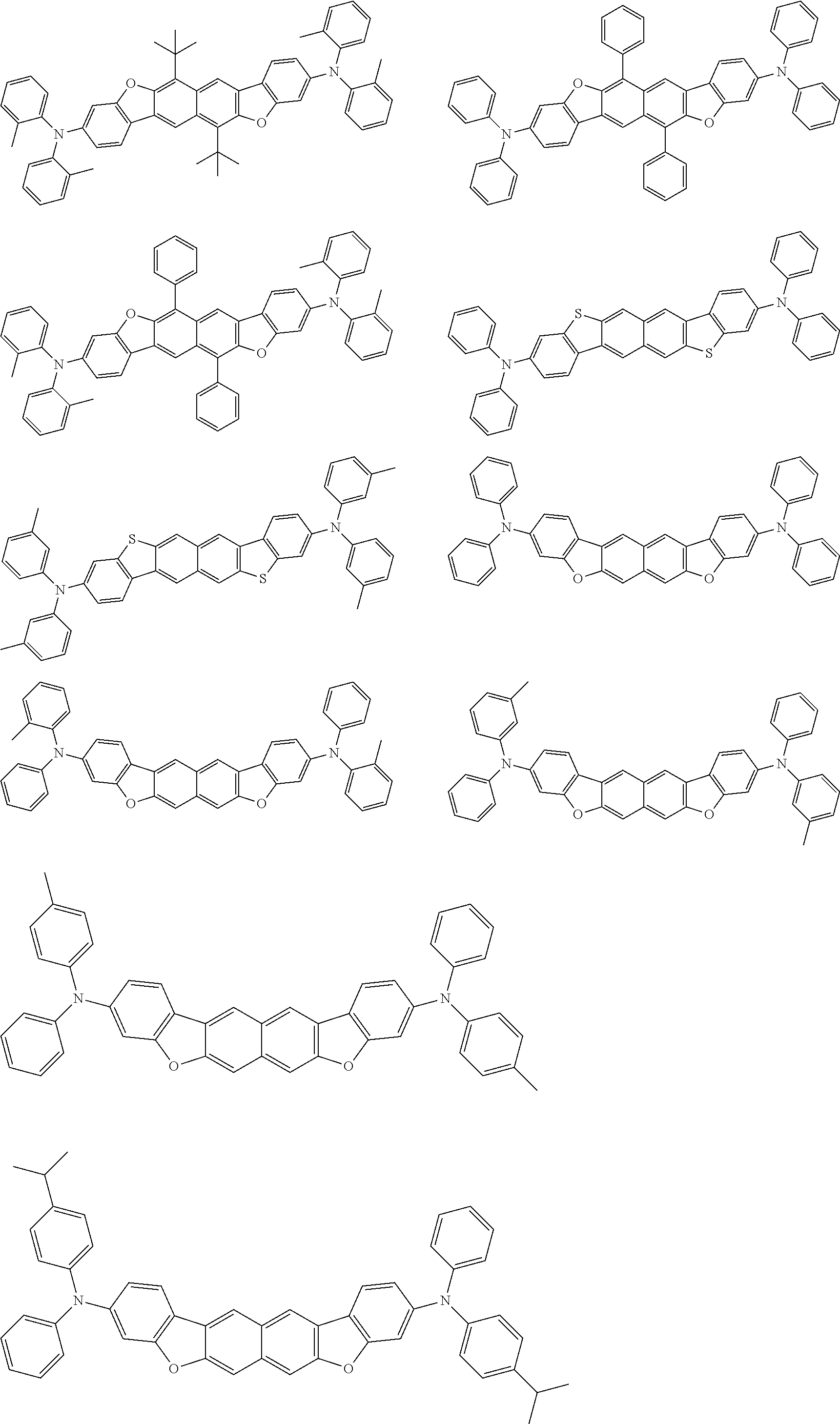

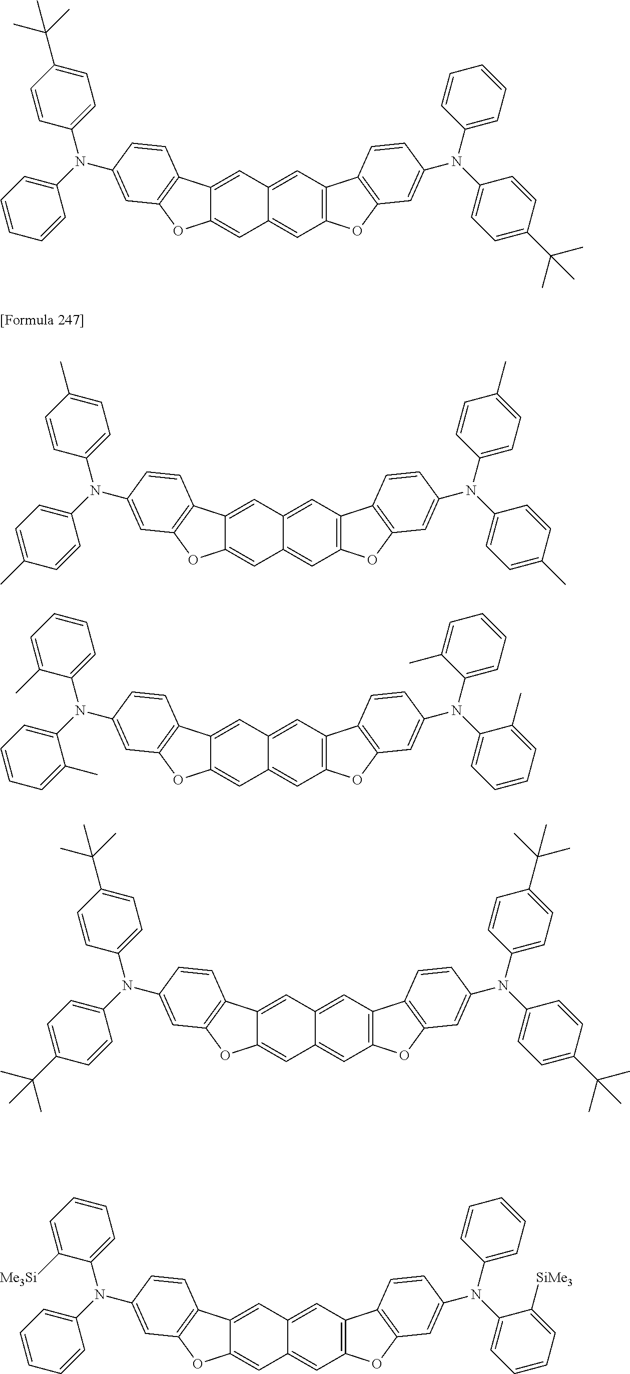

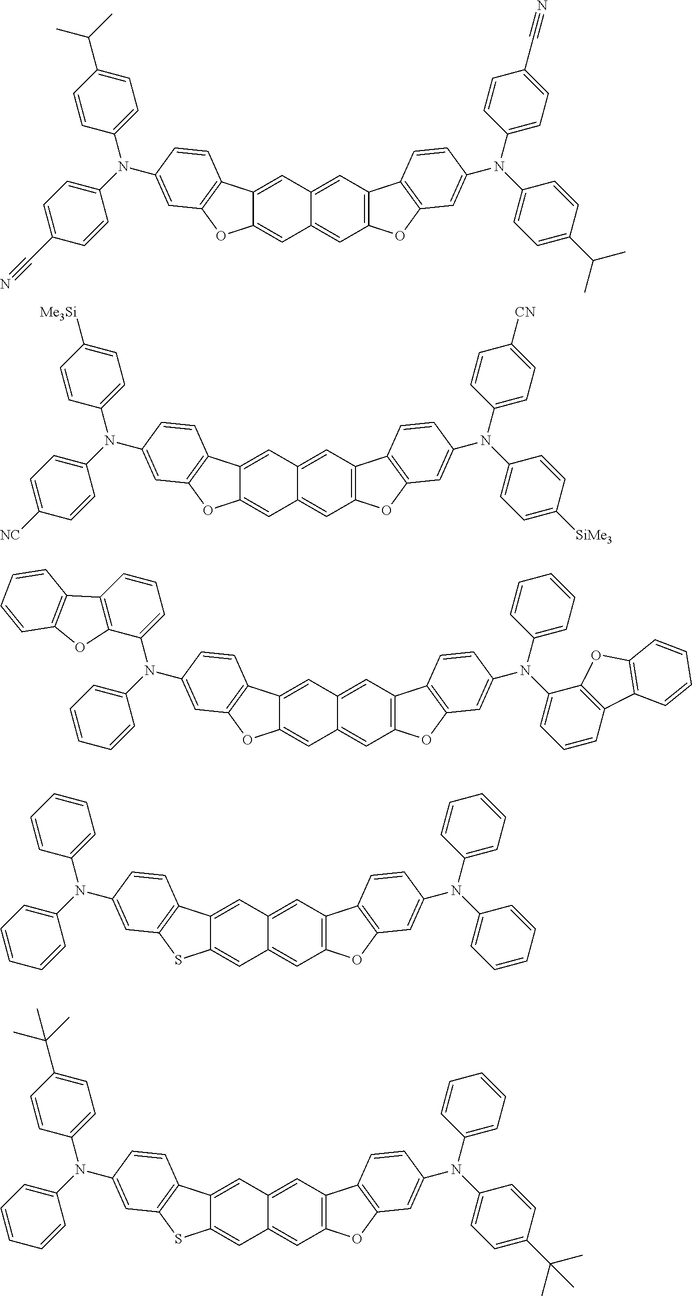

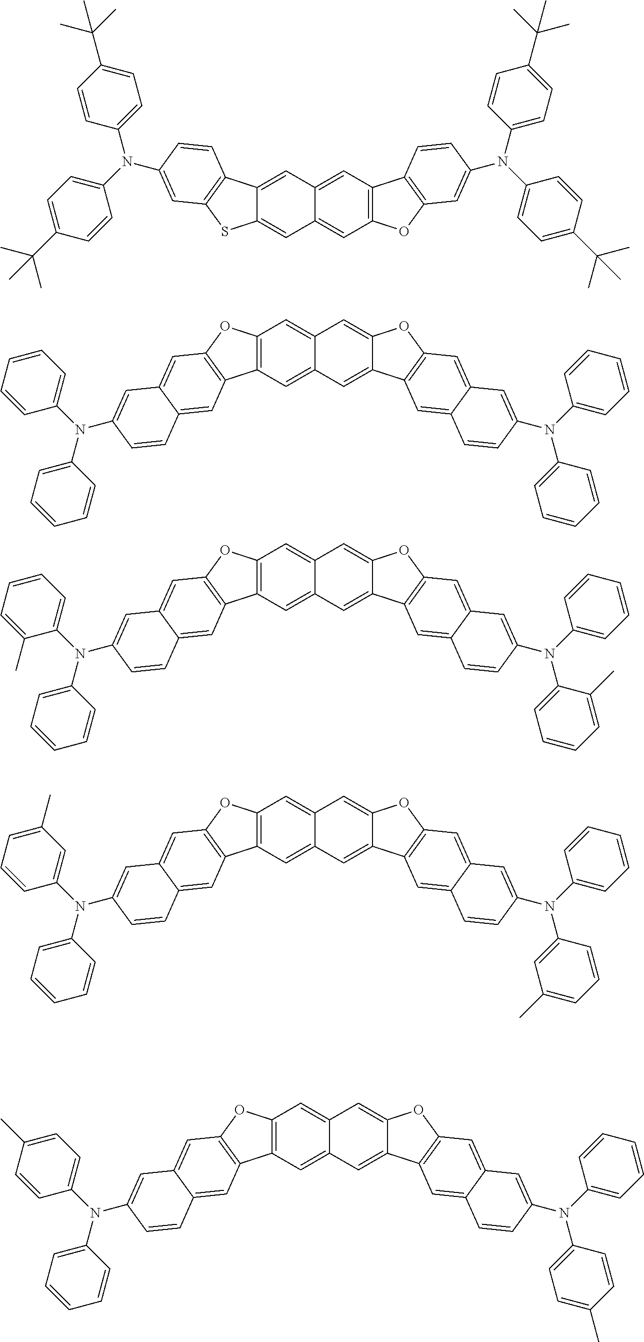

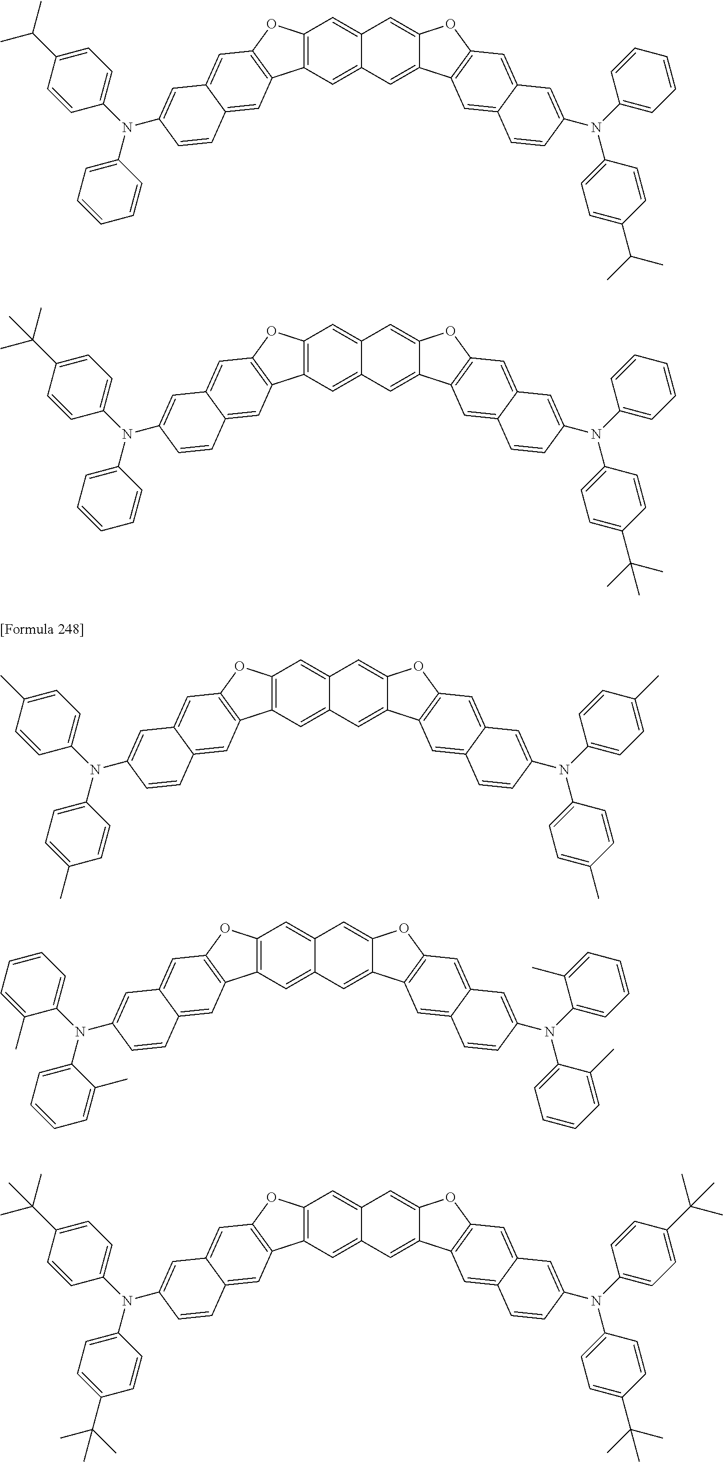

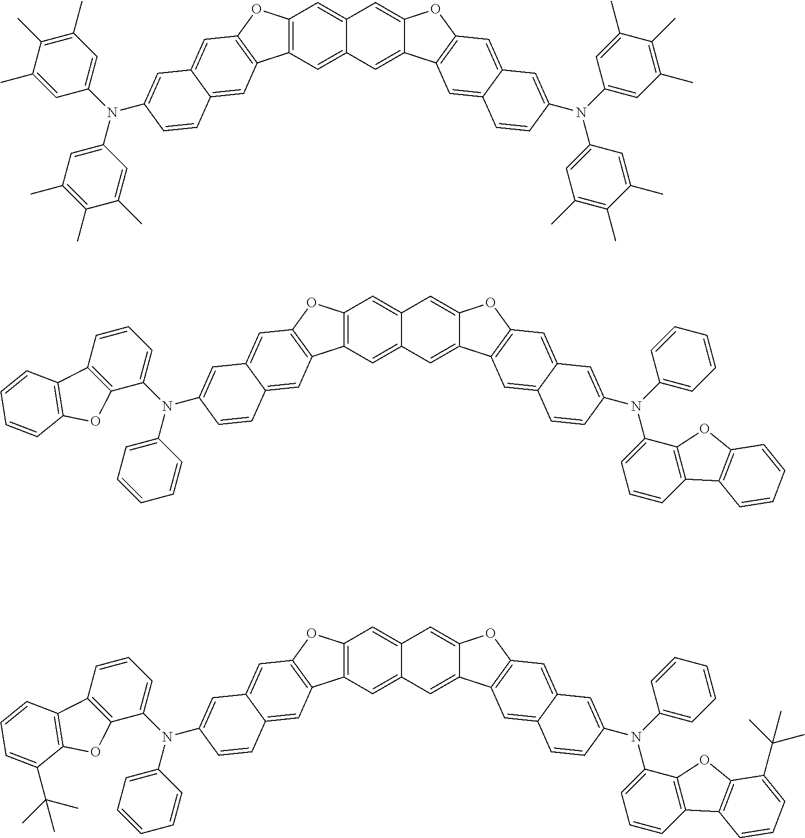

### Compound Represented by Formula (8)

The compound represented by the formula (8) will be described below.

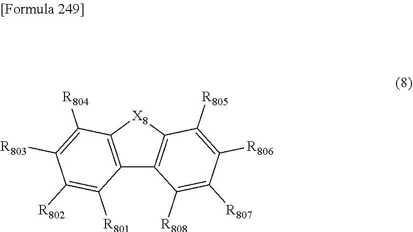

In the formula (8): at least one combination of R₈₀₁ and R₈₀₂, R₈₀₂ and R₈₀₃, or R₈₀₃ and R₈₀₄ are mutually bonded to form a divalent group represented by a formula (82) below, and

at least one combination of R₈₀₅ and R₈₀₆, R₈₀₆ and R₈₀₇, or R₈₀₇ and R₈₀₈ are mutually bonded to form a divalent group represented by a formula (83) below.

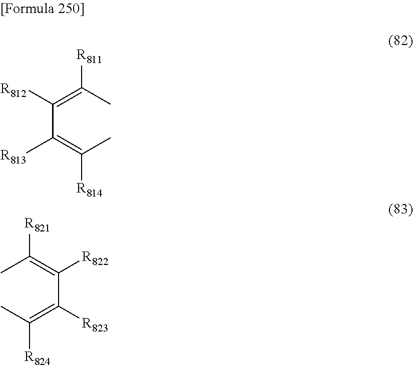

At least one of R₈₀₁ to R₈₀₄ not forming the divalent group represented by the formula (82) or R₈₁₁ to R₈₁₄ is a monovalent group represented by a formula (84) below.

At least one of R₈₀₅ to R₈₀₈ not forming the divalent group represented by the formula (83) or R₈₂₁ to R₈₂₄ is a monovalent group represented by a formula (84) below.

X₈ is an oxygen atom, a sulfur atom, or NR₈₀₉.

R₈₀₁ to R₈₀₈ not forming the divalent group represented by the formula (82) or (83) and not being the monovalent group represented by the formula (84), R₈₁₁ to R₈₁₄ and R₈₂₁ to R₈₂₄ not being the monovalent group represented by the formula (84), and R₈₀₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

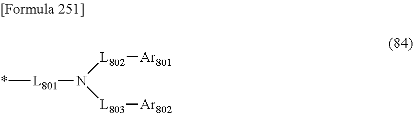

In the formula (84):

Ar₈₀₁ and Ar₈₀₂ are each independently a substituted or unsubstituted aryl group having 6 to 50 ring carbon atoms, or a substituted or unsubstituted heterocyclic group having 5 to 50 ring atoms;

L₈₀₁ to L₈₀₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and

* in the formula (84) represents a bonding position to a cyclic structure represented by the formula (8), a group represented by the formula (82), or a group represented by the formula (83).

In the formula (8), the positions for the divalent group represented by the formula (82) and the divalent group represented by the formula (83) to be formed are not specifically limited but the divalent groups may be formed at any possible positions on R₈₀₁ to R₈₀₈.

In an exemplary embodiment, the compound represented by the formula (8) is represented by any one of formulae (81-1) to (81-6) below.

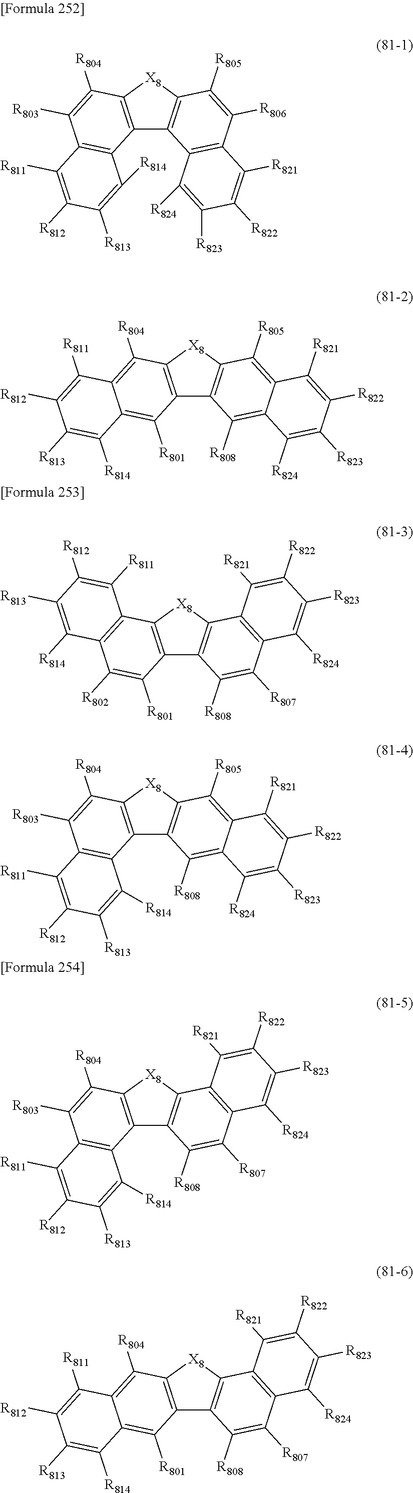

In the formulae (81-1) to (81-6):

X₈ represents the same as X₈ in the formula (8);

at least two of R₈₀₁ to R₈₂₄ are each a monovalent group represented by the formula (84); and

R₈₀₁ to R₈₂₄ that are not the monovalent group represented by the formula (84) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (8) is represented by any one of formulae (81-7) to (81-18) below.

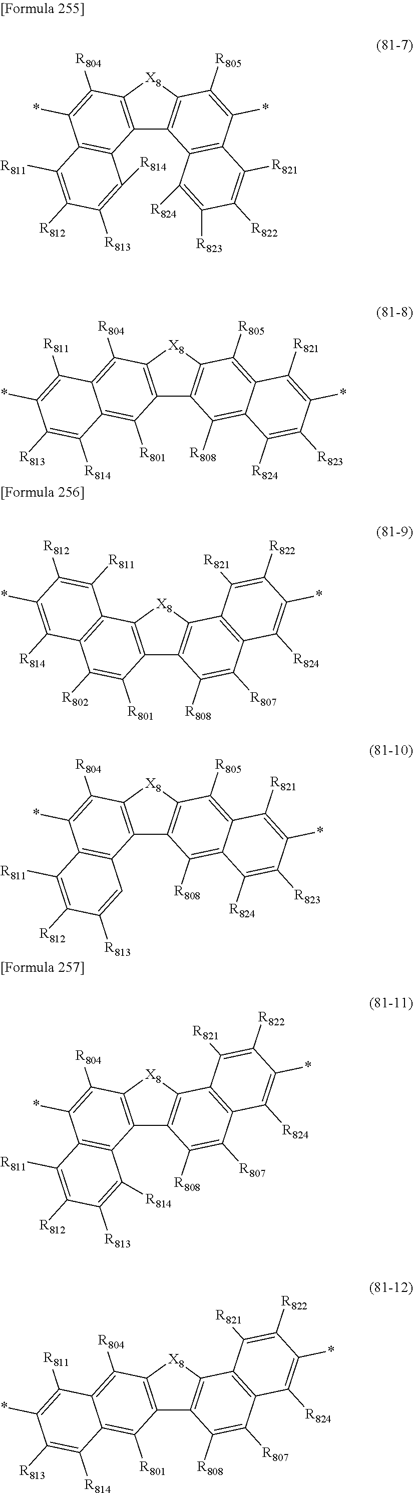

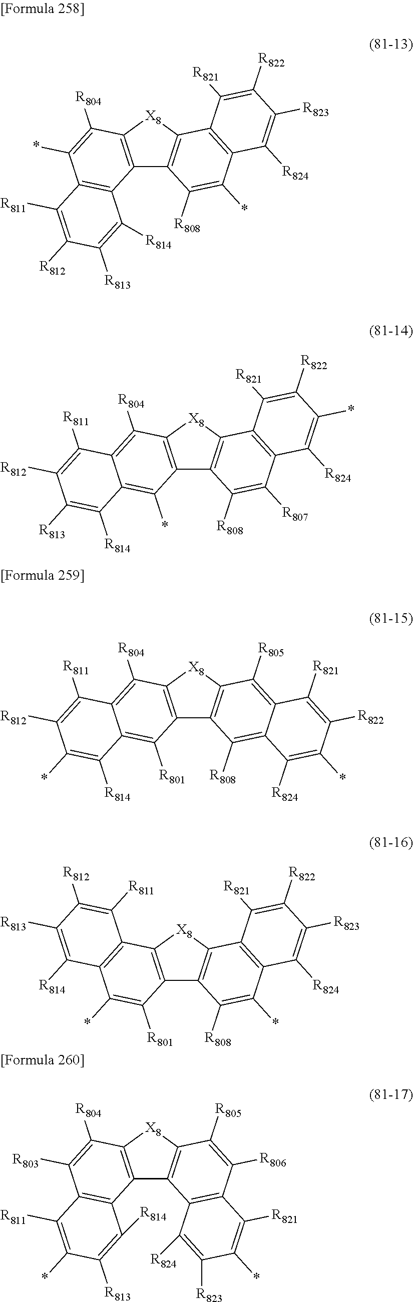

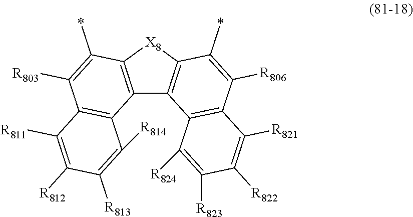

In the formulae (81-7) to (81-18):

X₈ represents the same as X₈ in the formula (8);

* is a single bond to be bonded with the monovalent group represented by the formula (84); and

R₈₀₁ to R₈₂₄ each independently represent the same as R₈₀₁ to R₈₂₄ in the formulae (81-1) to (81-6) that are not a monovalent group represented by the formula (84).

R₈₀₁ to R₈₀₈ not forming the divalent group represented by the formula (82) or (83) and not being the monovalent group represented by the formula (84), and R₈₁₁ to R₈₁₄ and R₈₂₁ to R₈₂₄ not being the monovalent group represented by the formula (84) are preferably each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

The monovalent group represented by the formula (84) is preferably represented by a formula (85) or (86) below.

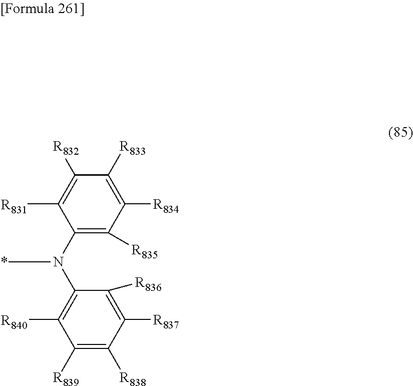

In the formula (85):

R₈₃₁ to R₈₄₀ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and

* in the formula (85) represents the same as * in the formula (84).

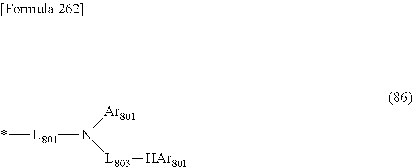

In the formula (86):

Ar₈₀₁, L₈₀₁, and L₈₀₃ represent the same as Ar₈₀₁, L₈₀₁, and L₈₀₃ in the formula (84); and

HAr₈₀₁ is a structure represented by a formula (87) below.

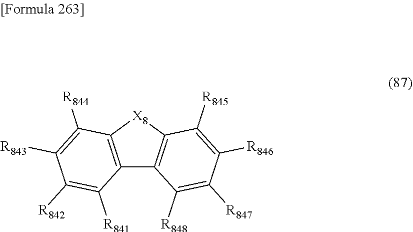

In the formula (87):

X₈₁ is an oxygen atom or a sulfur atom;

one of R₈₄₁ to R₈₄₈ is a single bond with L₈₀₃; and

R₈₄₁ to R₈₄₈ not being the single bond are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

Specific examples of the compound represented by the formula (8) include compounds shown below as well as the compounds disclosed in WO 2014/104144.

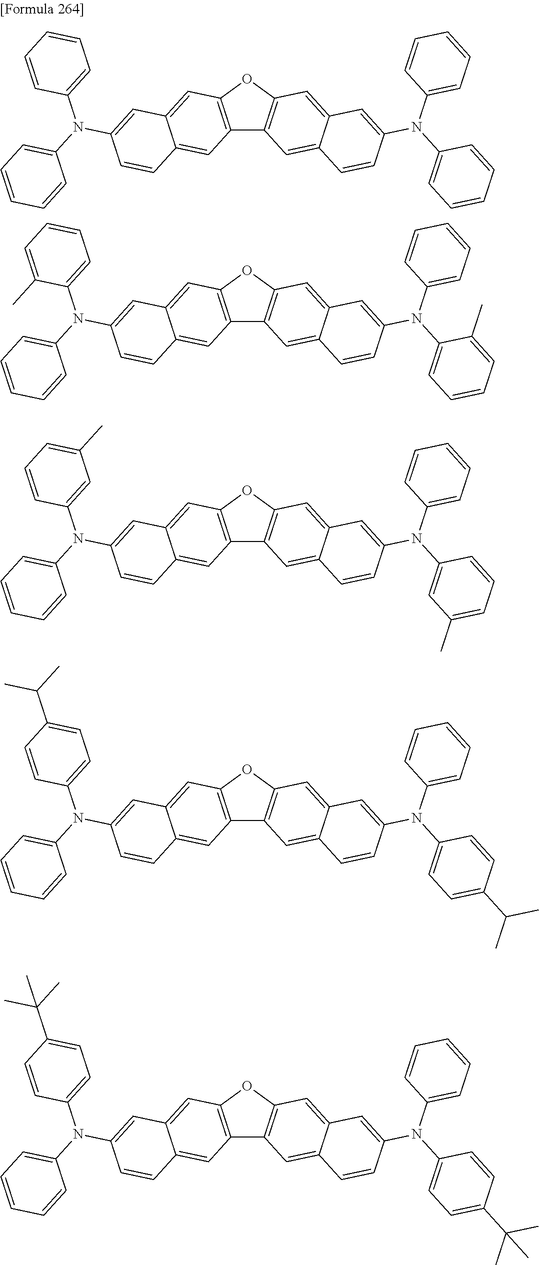

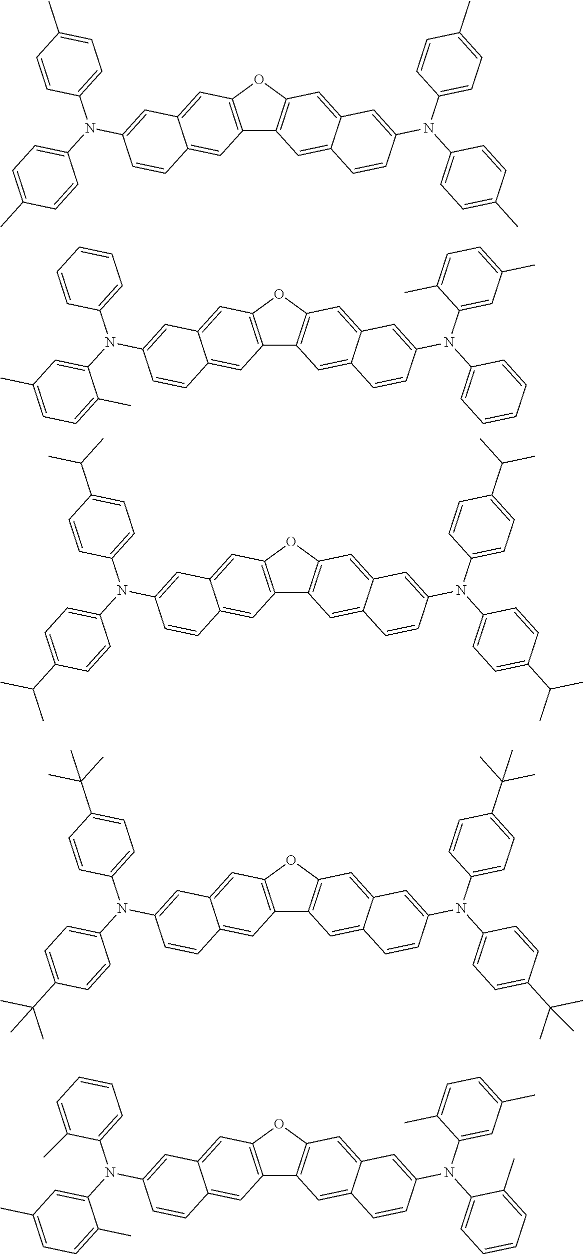

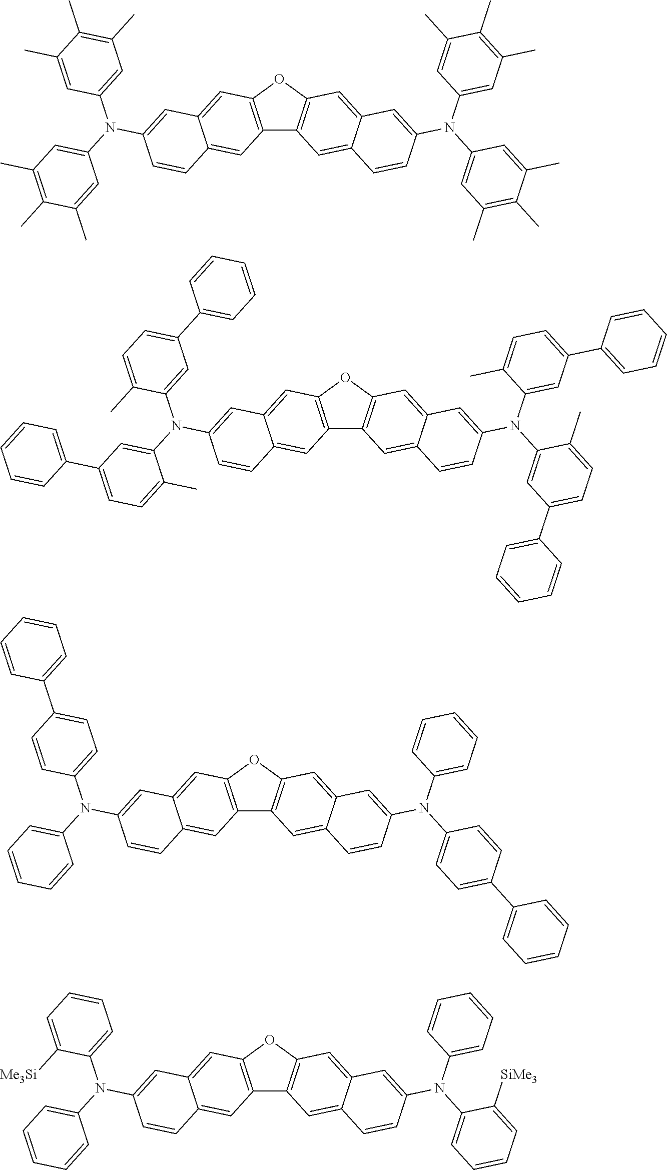

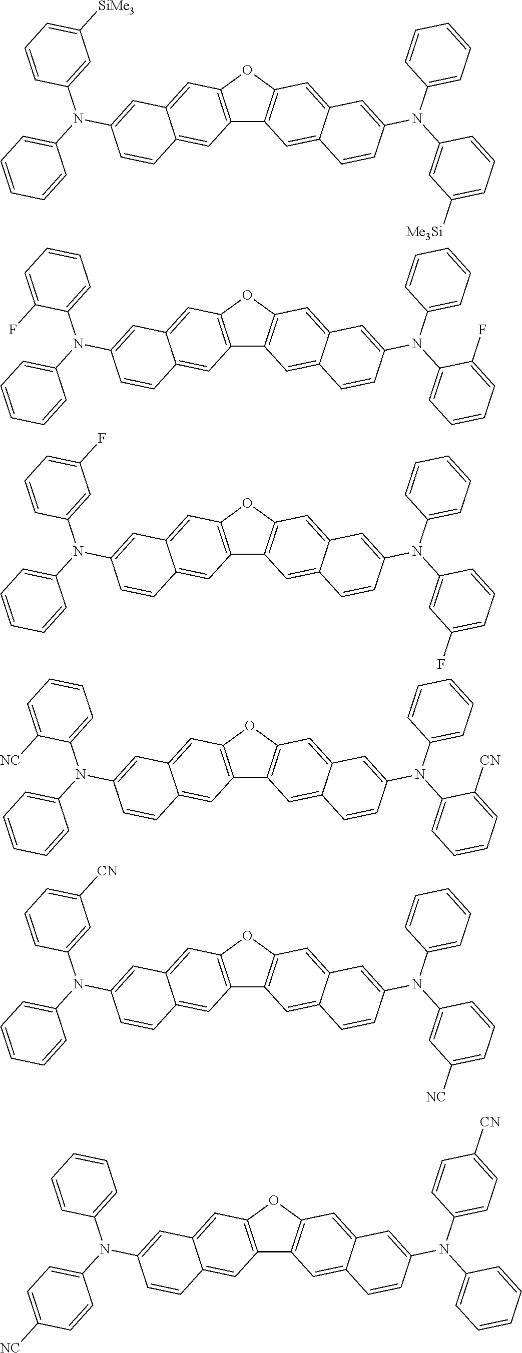

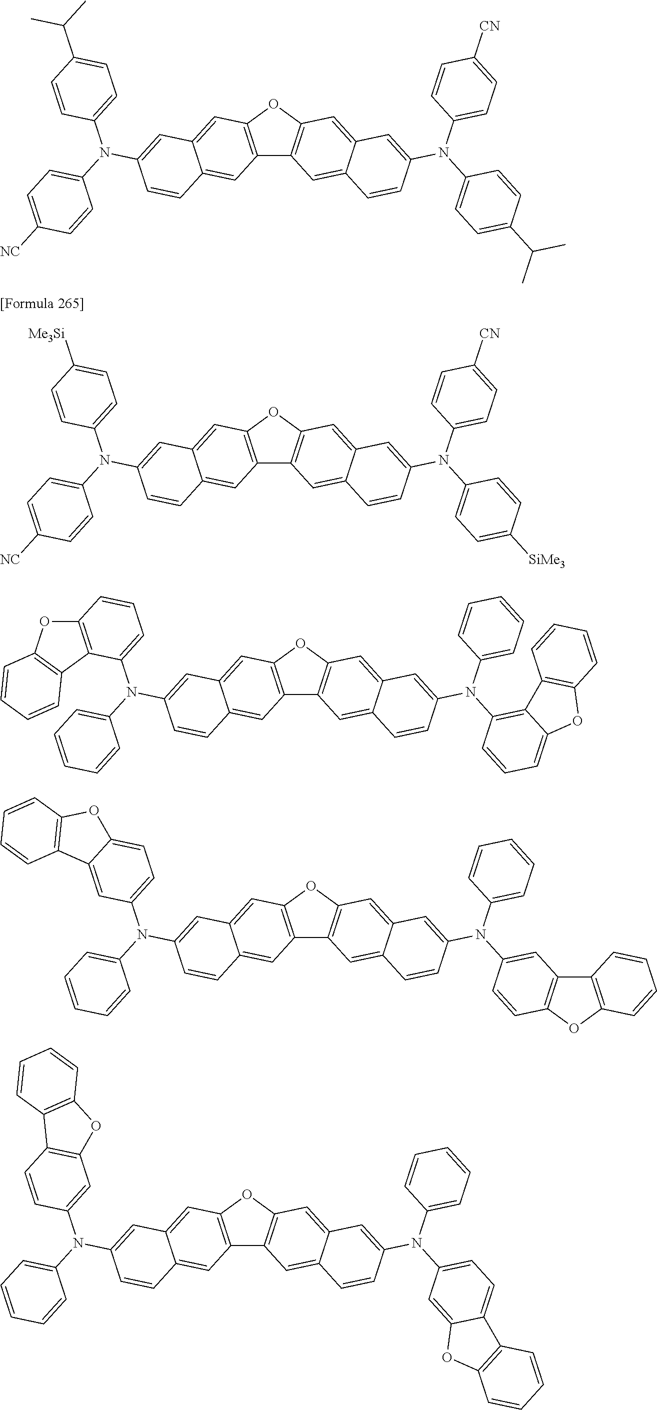

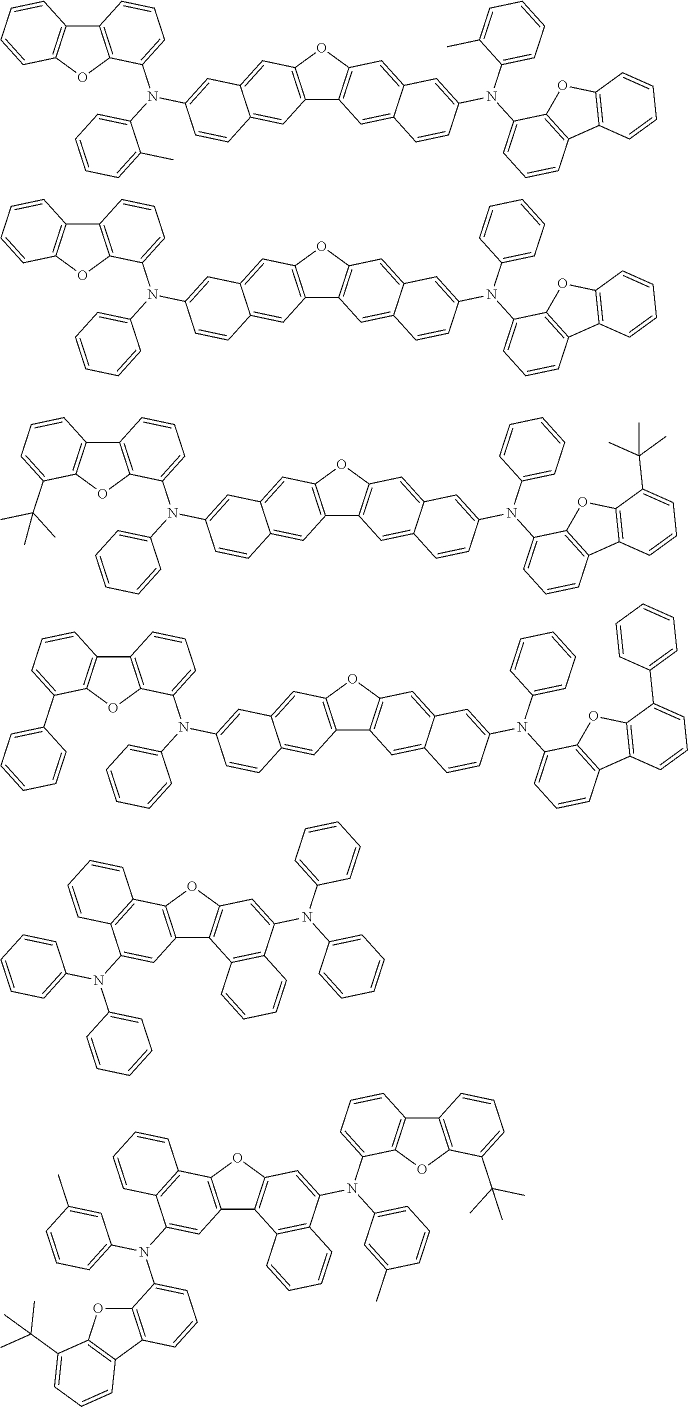

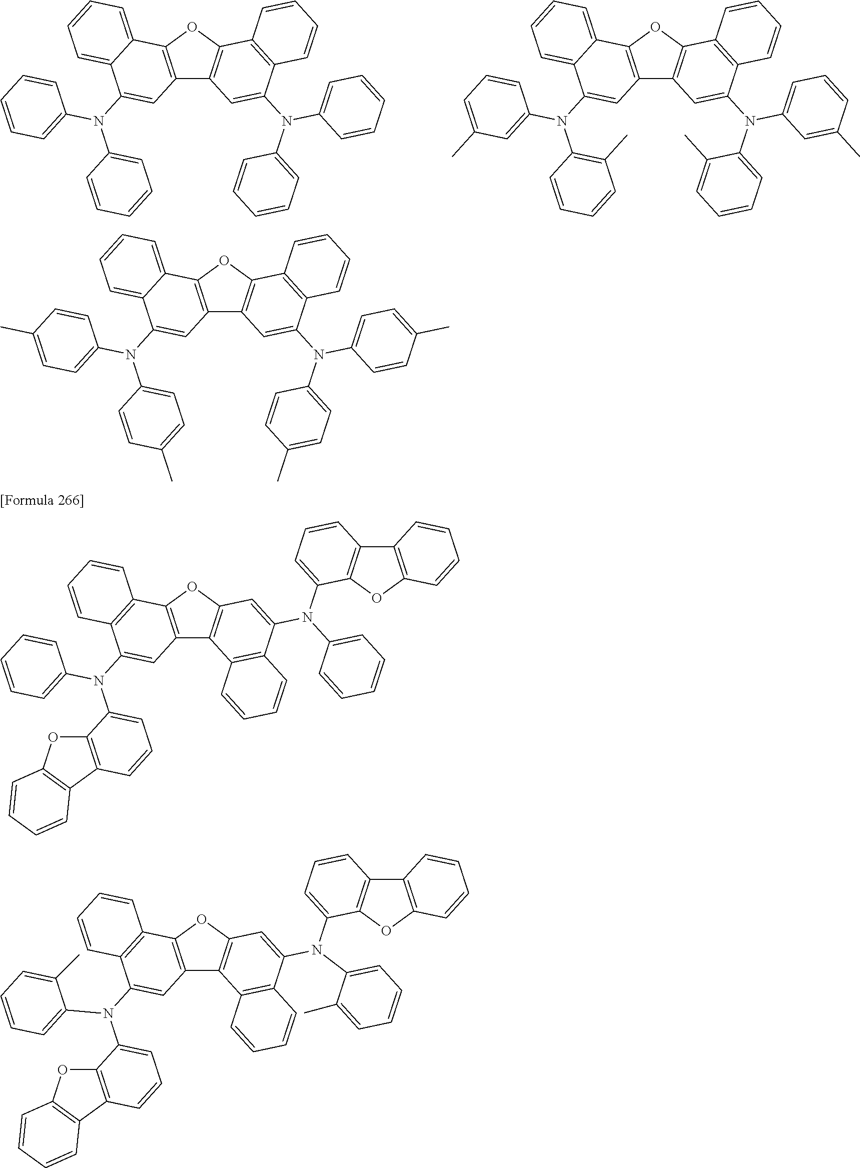

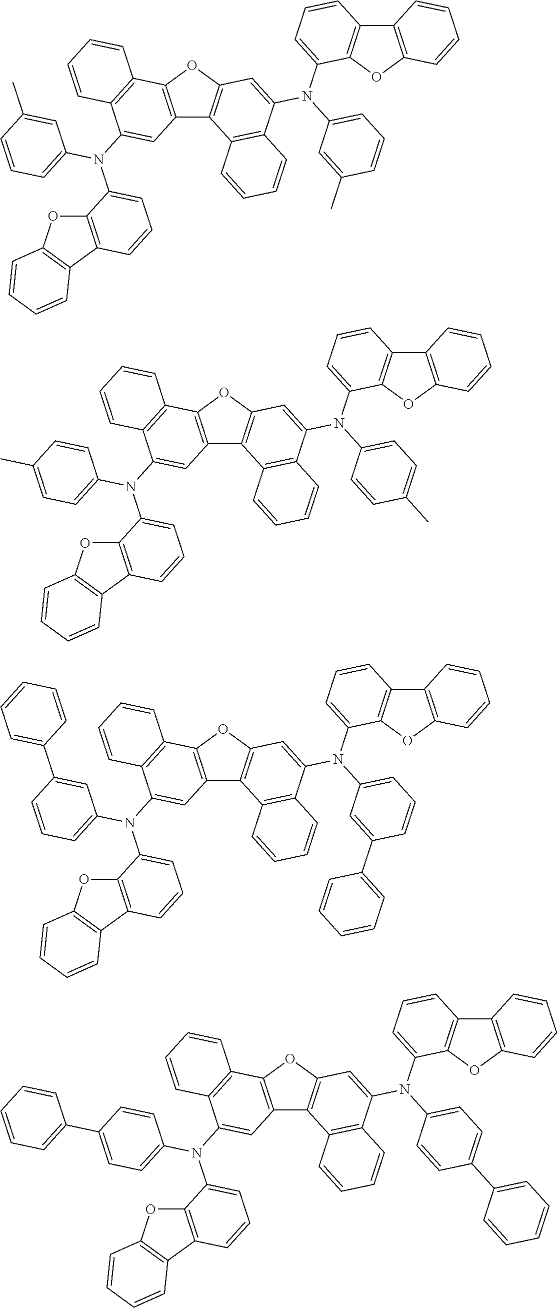 [SMILES: Cc1cccc(N(c2cc3c(oc4cc(N(c5cccc(C)c5)c5cccc6c5oc5ccccc56)c5ccccc5c43)c3ccccc23)c2cccc3c2oc2ccccc23)c1 ; Cc1ccc(N(c2cc3c(oc4cc(N(c5ccc(C)cc5)c5cccc6c5oc5ccccc56)c5ccccc5c43)c3ccccc23)c2cccc3c2oc2ccccc23)cc1 ; c1ccc(-c2cccc(N(c3cc4c(oc5cc(N(c6cccc(-c7ccccc7)c6)c6cccc7c6oc6ccccc67)c6ccccc6c54)c4ccccc34)c3cccc4c3oc3ccccc34)c2)cc1 ; c1ccc(-c2ccc(N(c3cc4c(oc5cc(N(c6ccc(-c7ccccc7)cc6)c6cccc7c6oc6ccccc67)c6ccccc6c54)c4ccccc34)c3cccc4c3oc3ccccc34)cc2)cc1]

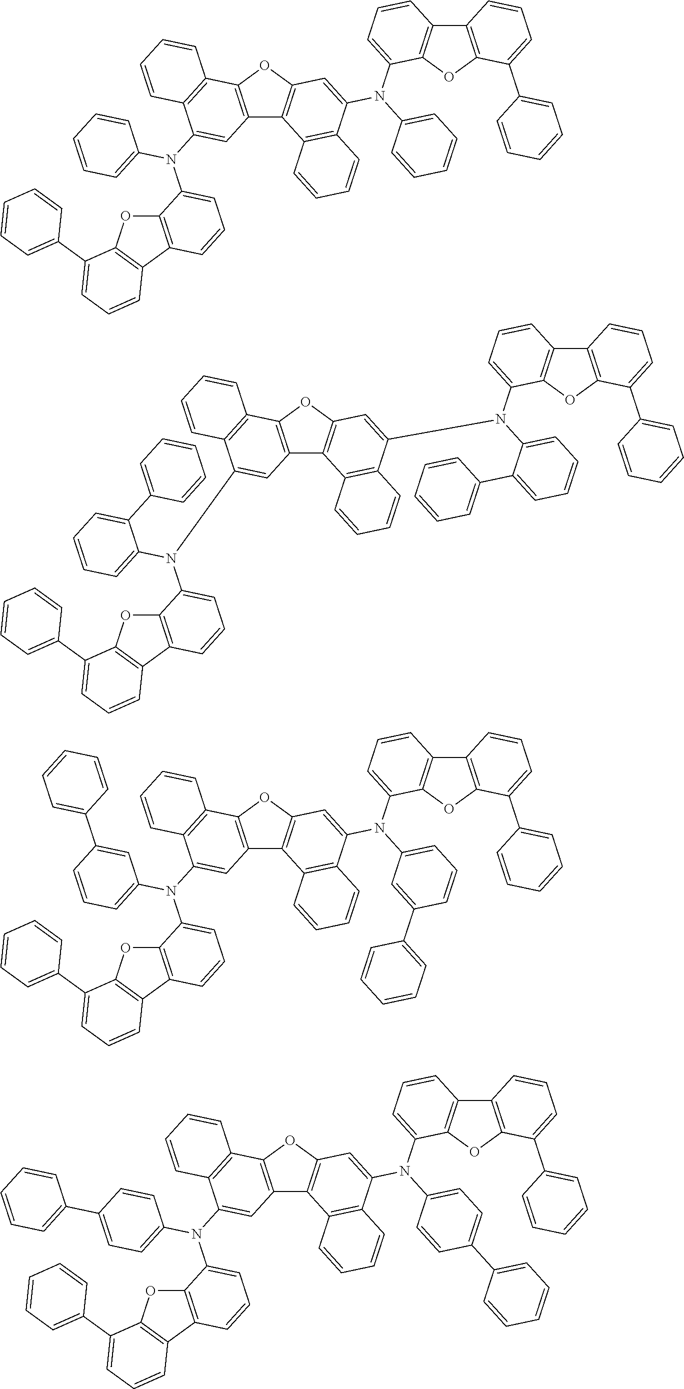

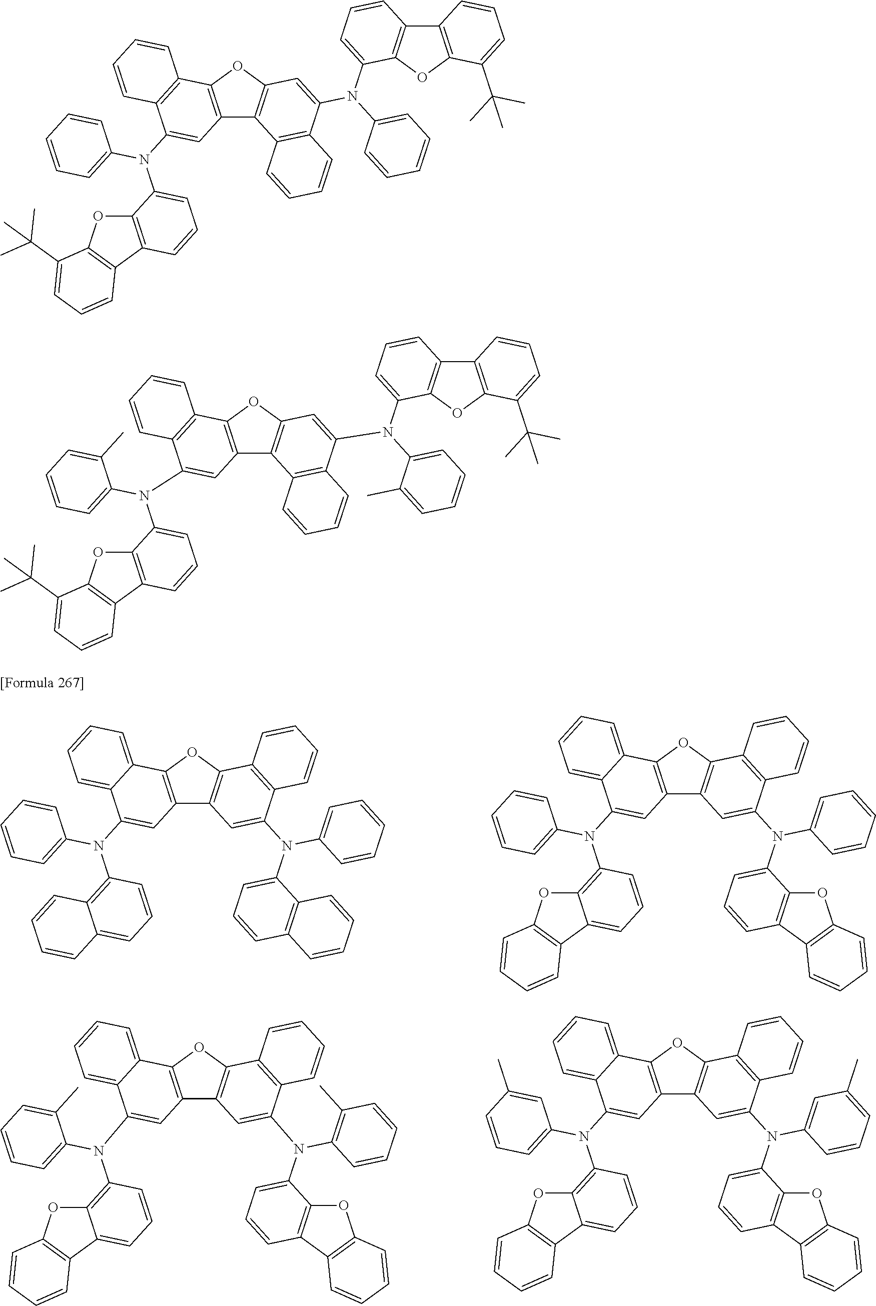

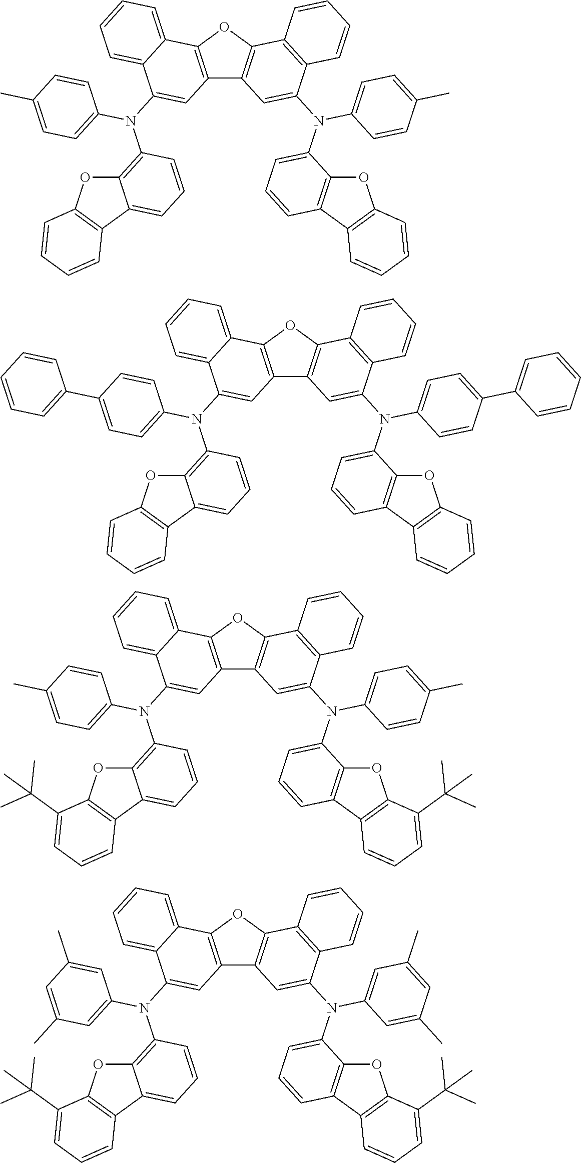

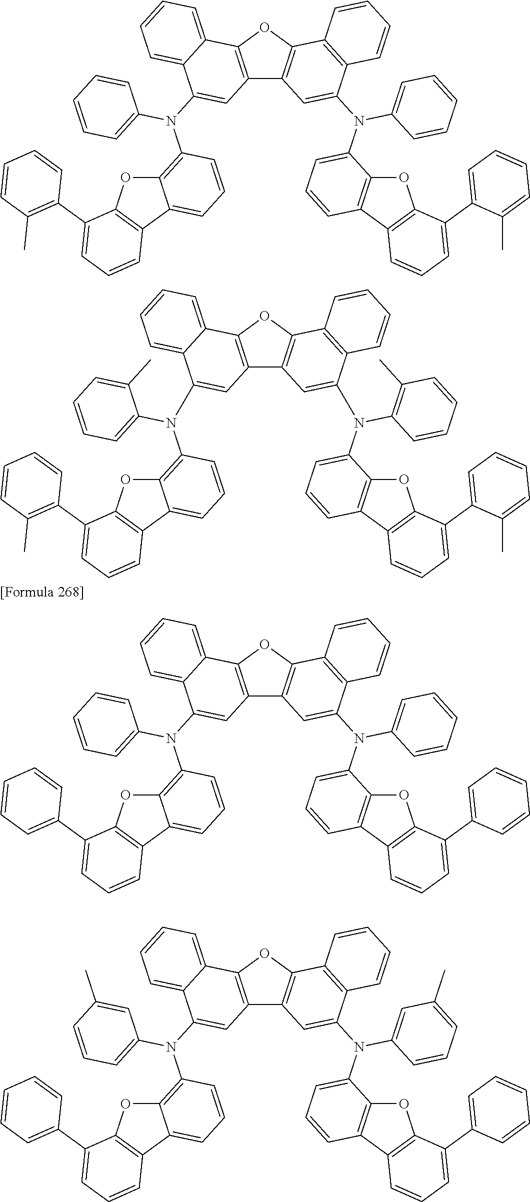

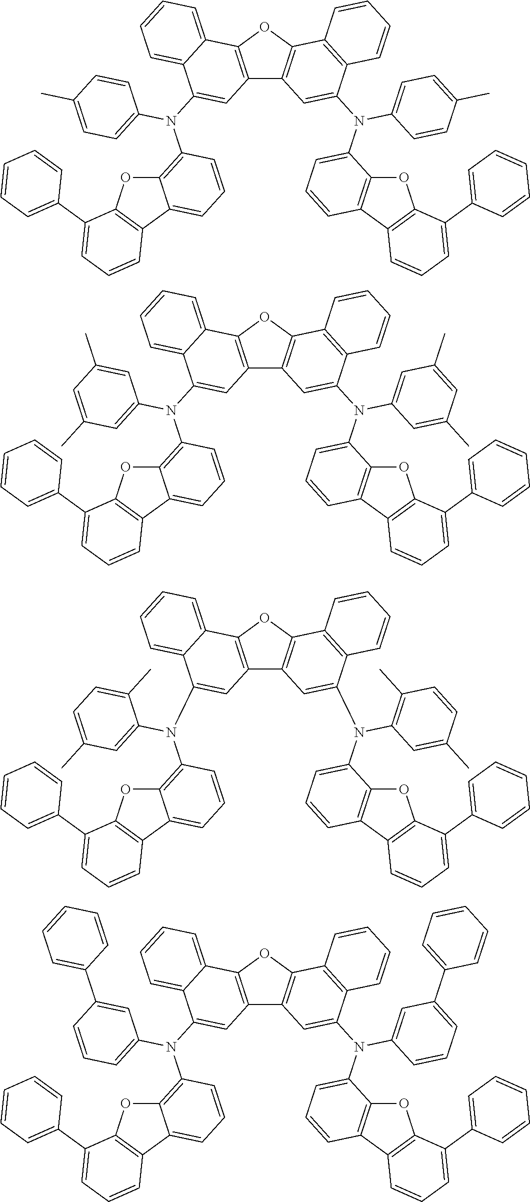

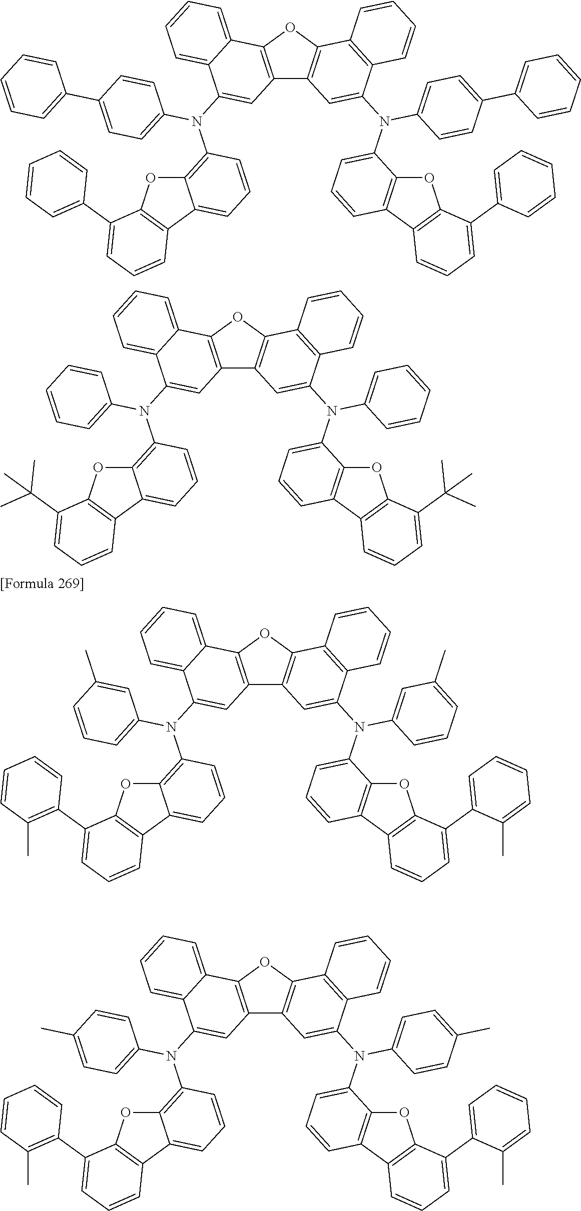

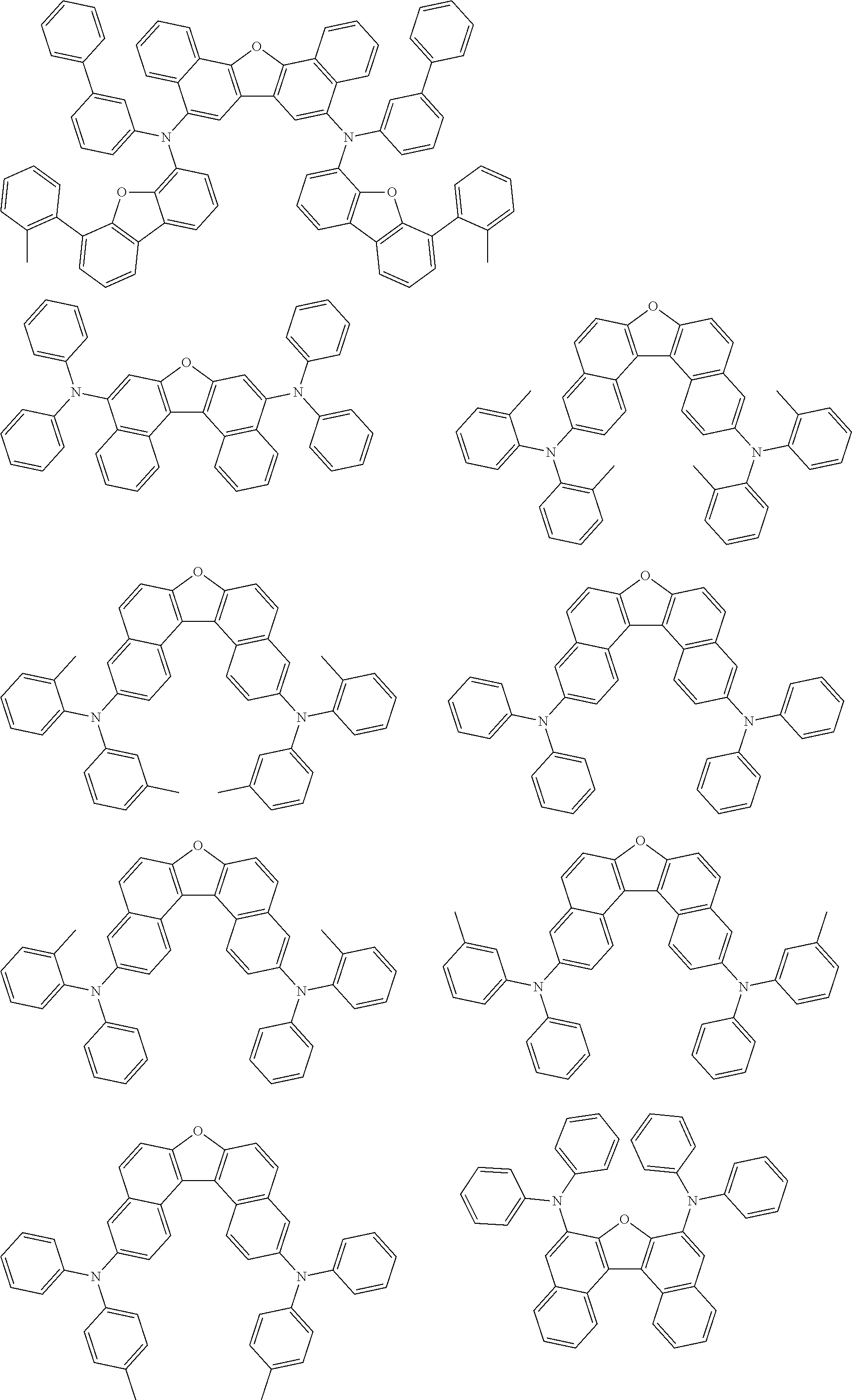

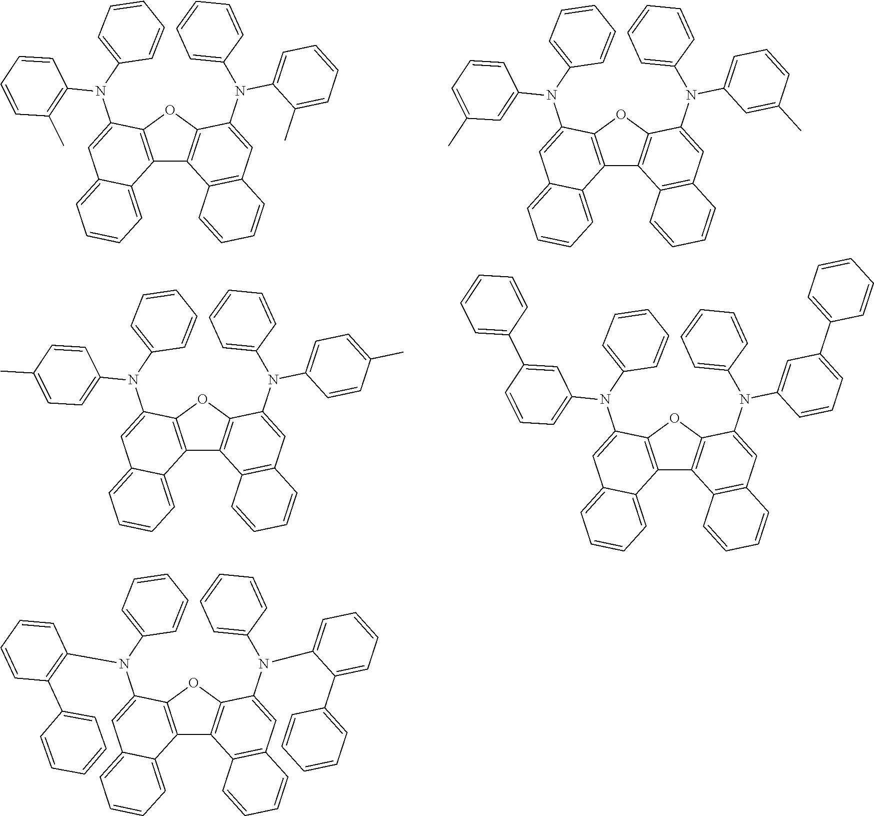

### Compound Represented by Formula (9)

The compound represented by the formula (9) will be described below.

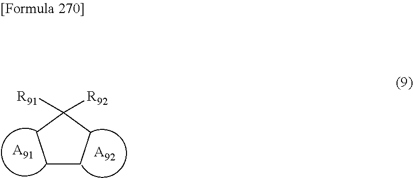

In the formula (9):

A₉₁ ring and A₉₂ ring are each independently a substituted or unsubstituted aromatic hydrocarbon ring having 6 to 50 ring carbon atoms, or a substituted or unsubstituted heterocycle having 5 to 50 ring atoms; and

at least one of A₉₁ ring or A₉₂ ring is bonded to a structure represented by a formula (92) below.

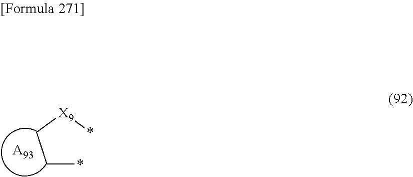

In the formula (92):

A₉₃ ring is a substituted or unsubstituted aromatic hydrocarbon ring having 6 to 50 ring carbon atoms, or a substituted or unsubstituted heterocycle having 5 to 50 ring atoms;

X₉ is NR₉₃, C(R₉₄)(R₉₅), Si(R₉₆)(R₉₇), Ge(R₉₈)(R₉₉), an oxygen atom, a sulfur atom, or a selenium atom;

R₉₁ and R₉₂ are mutually bonded to form a substituted or unsubstituted monocyclic ring, mutually bonded to form a substituted or unsubstituted fused ring, or not mutually bonded; and

R₉₁ and R₉₂ not forming the monocyclic ring and not forming the fused ring, and R₉₃ to R₉₉ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

At least one ring selected from the group consisting of A₉₁ ring and A₉₂ ring is bonded to * of the structure represented by the formula (92). In other words, the ring-forming carbon atoms of the aromatic hydrocarbon ring or the ring atoms of the heterocycle of the A₉₁ ring in an exemplary embodiment are bonded to * in the structure represented by the formula (92). Further, the ring-forming carbon atoms of the aromatic hydrocarbon ring or the ring atoms of the heterocycle of the A₉₂ ring in an exemplary embodiment are bonded to * in the structure represented by the formula (92).

In an exemplary embodiment, a group represented by a formula (93) below is bonded to one or both of the A₉₁ ring and A₉₂ ring.

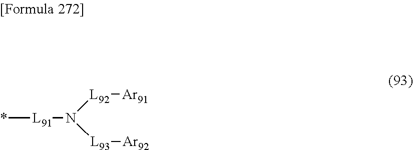

In the formula (93):

Ar₉₁ and Ar₉₂ are each independently a substituted or unsubstituted aryl group having 6 to 50 ring carbon atoms, or a substituted or unsubstituted heterocyclic group having 5 to 50 ring atoms;

L₉₁ to L₉₃ are each independently a single bond, a substituted or unsubstituted arylene group having 6 to 30 ring carbon atoms, a substituted or unsubstituted divalent heterocyclic group having 5 to 30 ring atoms, or a divalent linking group formed by bonding two, three or four groups selected from the group consisting of a substituted or unsubstituted arylene group having 6 to 30 ring carbon atoms and a substituted or unsubstituted divalent heterocyclic group having 5 to 30 ring atoms; and

* in the formula (93) represents a bonding position to one of A₉₁ ring and A₉₂ ring.

In an exemplary embodiment, in addition to the A₉₁ ring, the ring-forming carbon atoms of the aromatic hydrocarbon ring or the ring atoms of the heterocycle of the A₉₂ ring are bonded to * in the moiety represented by the formula (92). In this case, the moieties represented by the formula (92) may be mutually the same or different.

In an exemplary embodiment, R₉₁ and R₉₂ are each independently a substituted or unsubstituted aryl group having 6 to 50 ring carbon atoms.

In an exemplary embodiment, R₉₁ and R₉₂ are mutually bonded to form a fluorene structure.

In an exemplary embodiment, the rings A₉₁ and A₉₂ are each independently a substituted or unsubstituted aromatic hydrocarbon ring having 6 to 50 ring carbon atoms, example of which is a substituted or unsubstituted benzene ring.

In an exemplary embodiment, the ring A₉₃ is a substituted or unsubstituted aromatic hydrocarbon ring having 6 to 50 ring carbon atoms, example of which is a substituted or unsubstituted benzene ring.

In an exemplary embodiment, X₉ is an oxygen atom or a sulfur atom.

Specific examples of the compound represented by the formula (9) include compounds shown below.

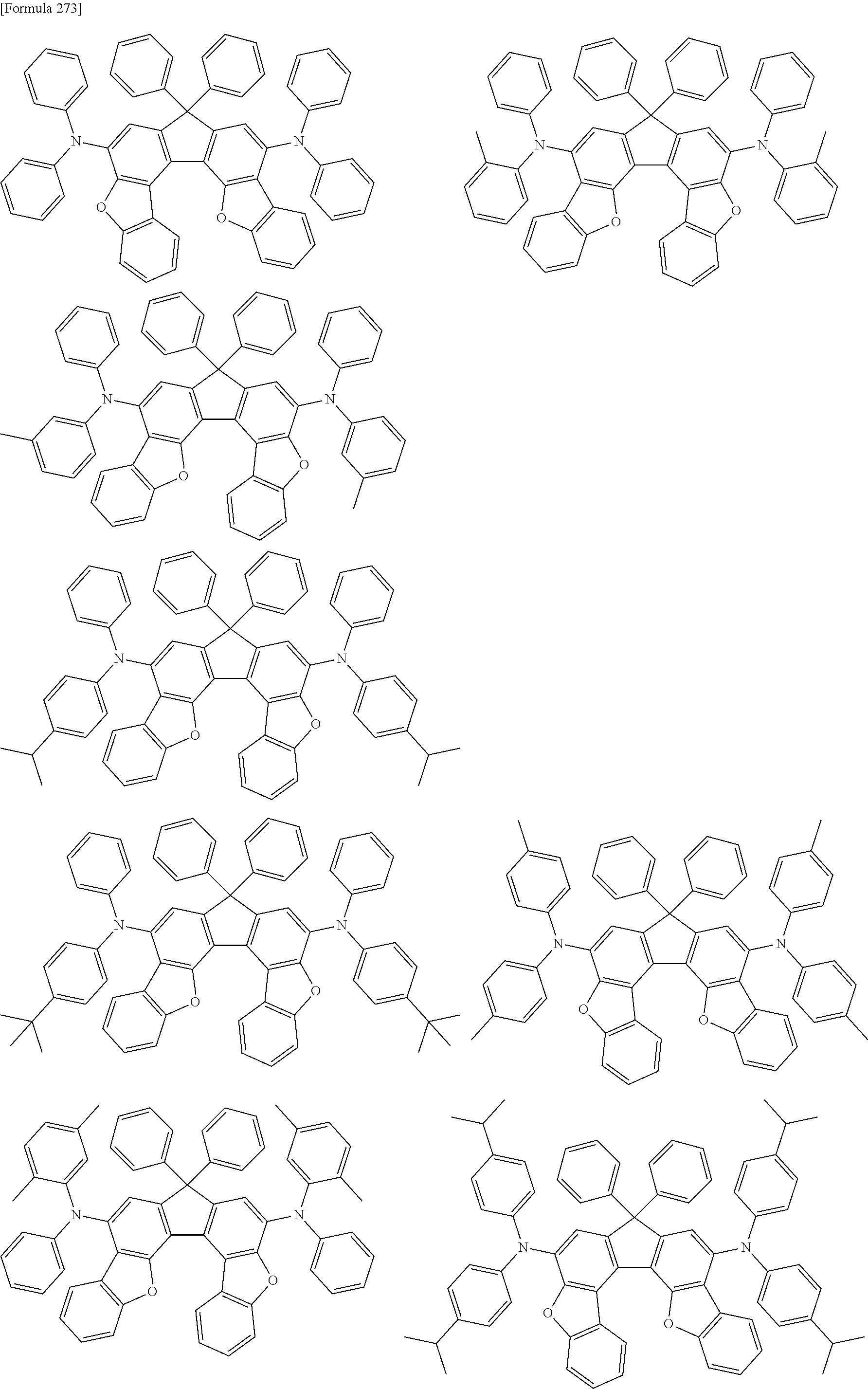

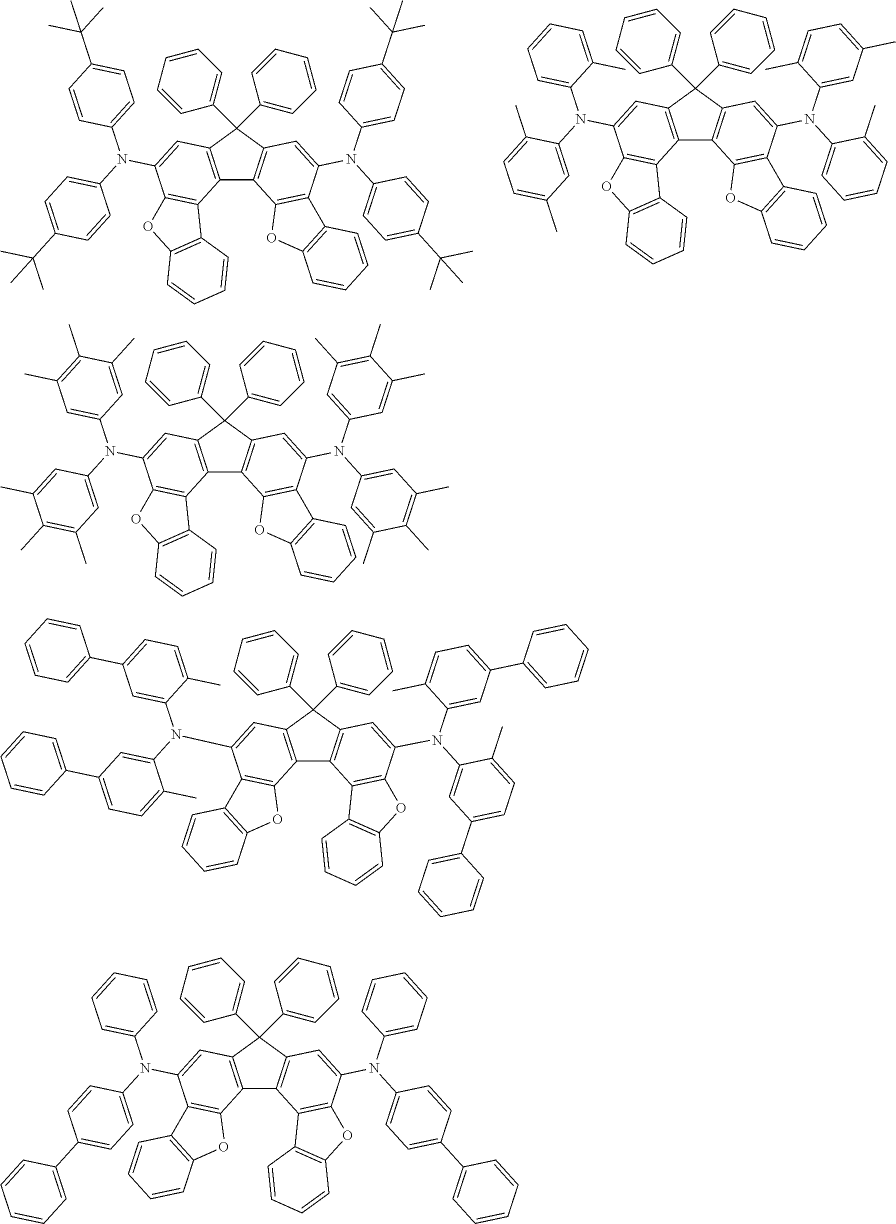

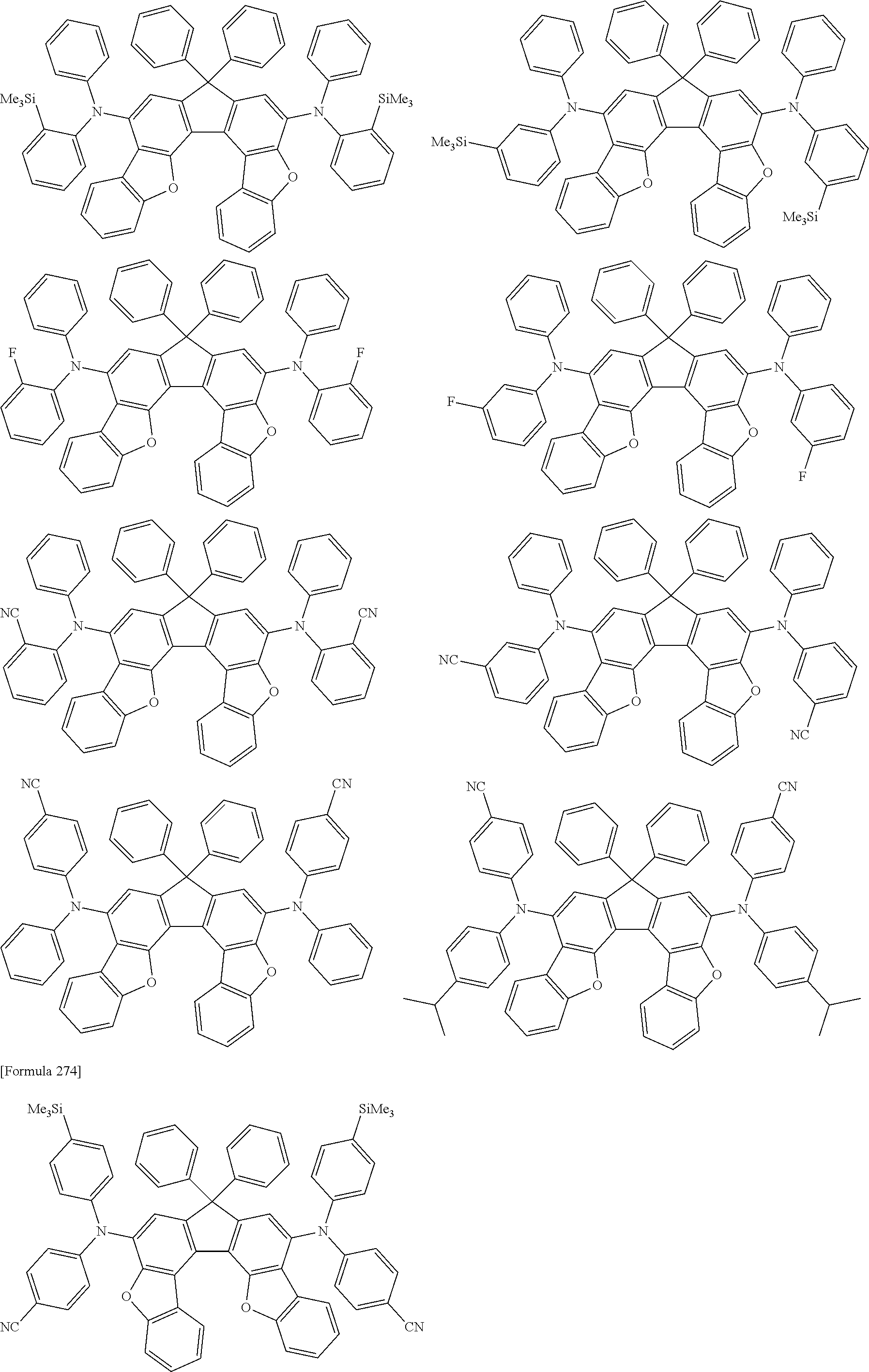

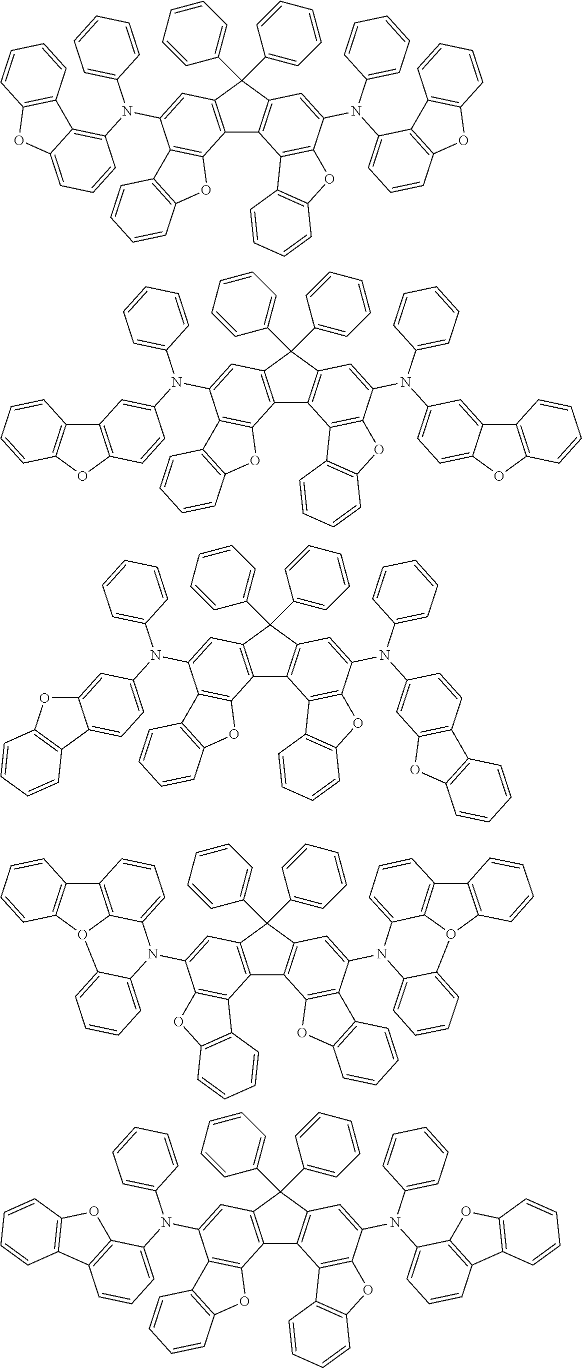

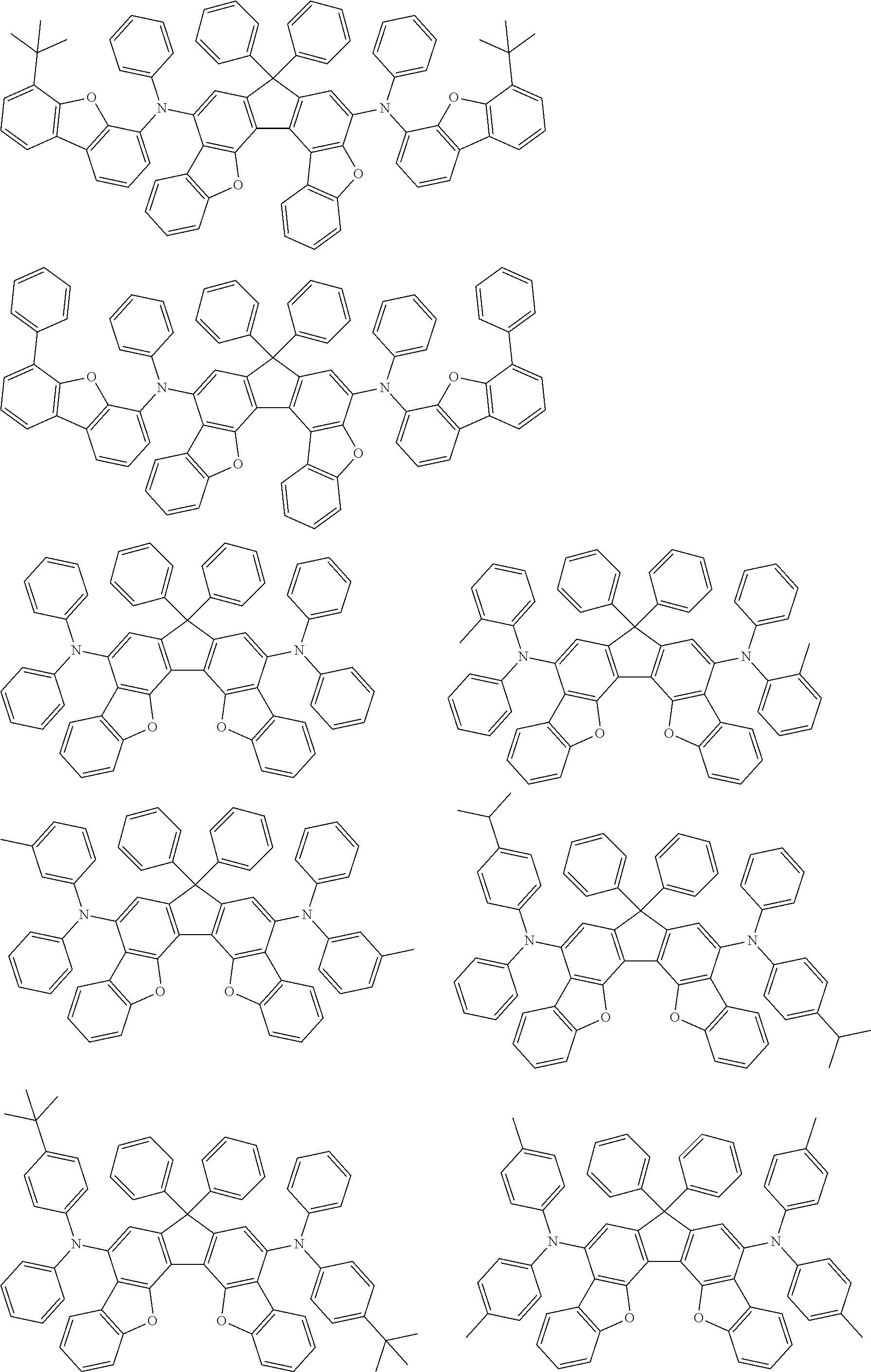

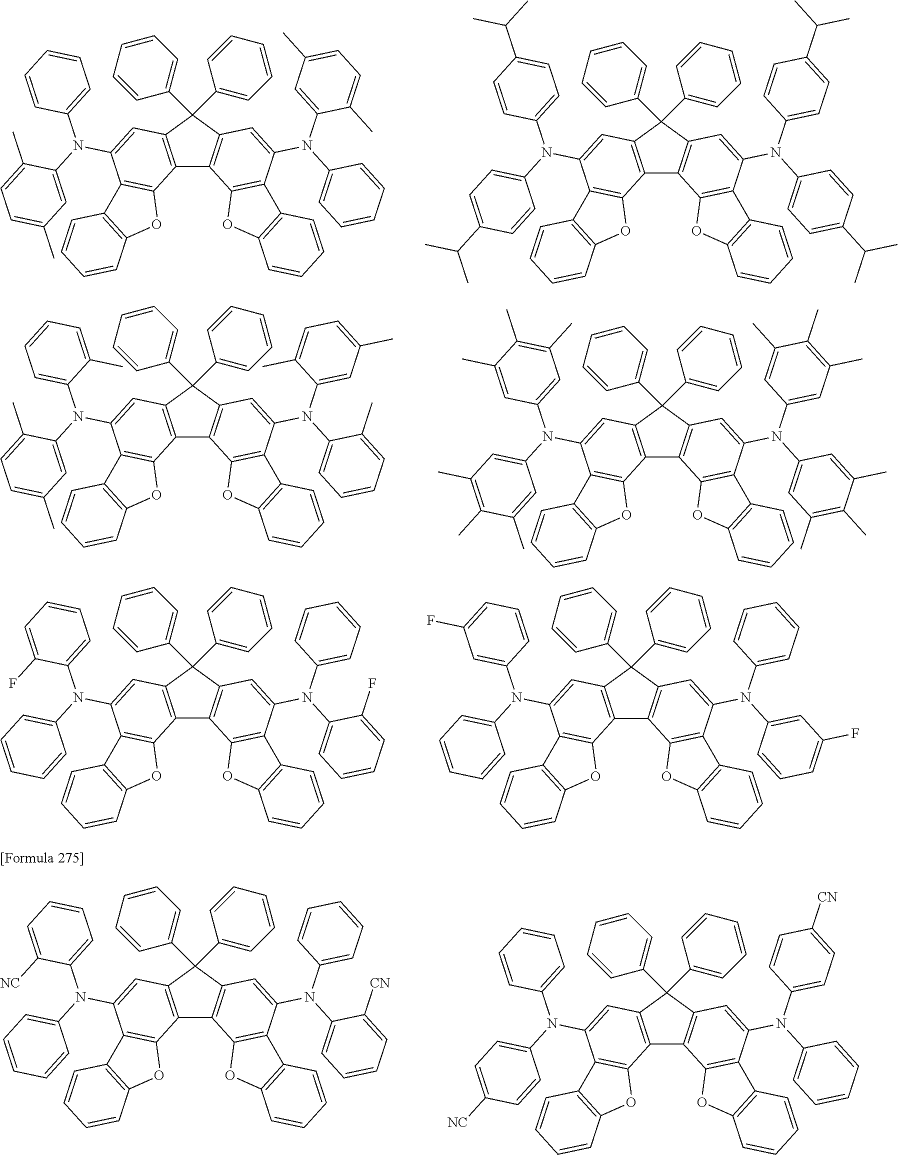

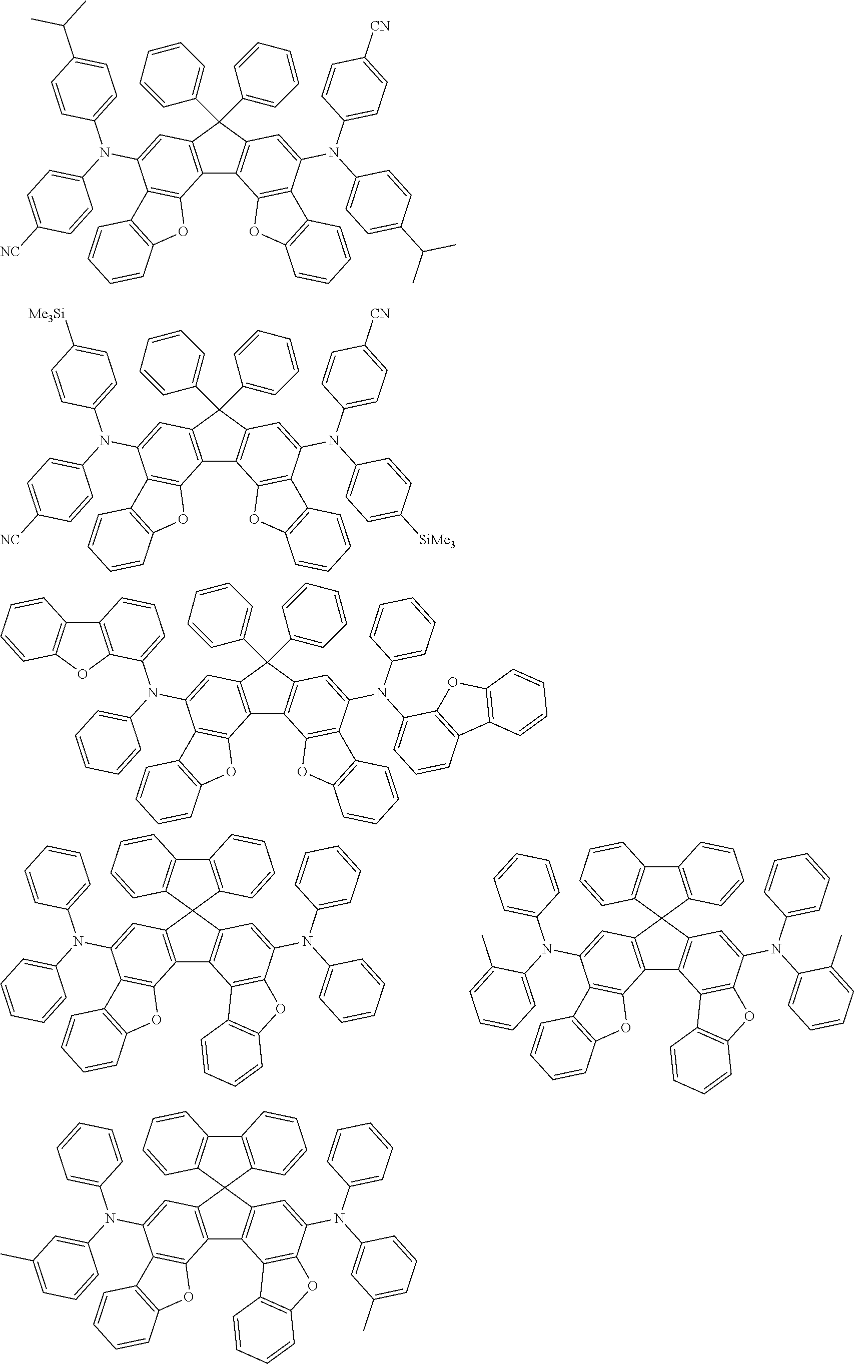

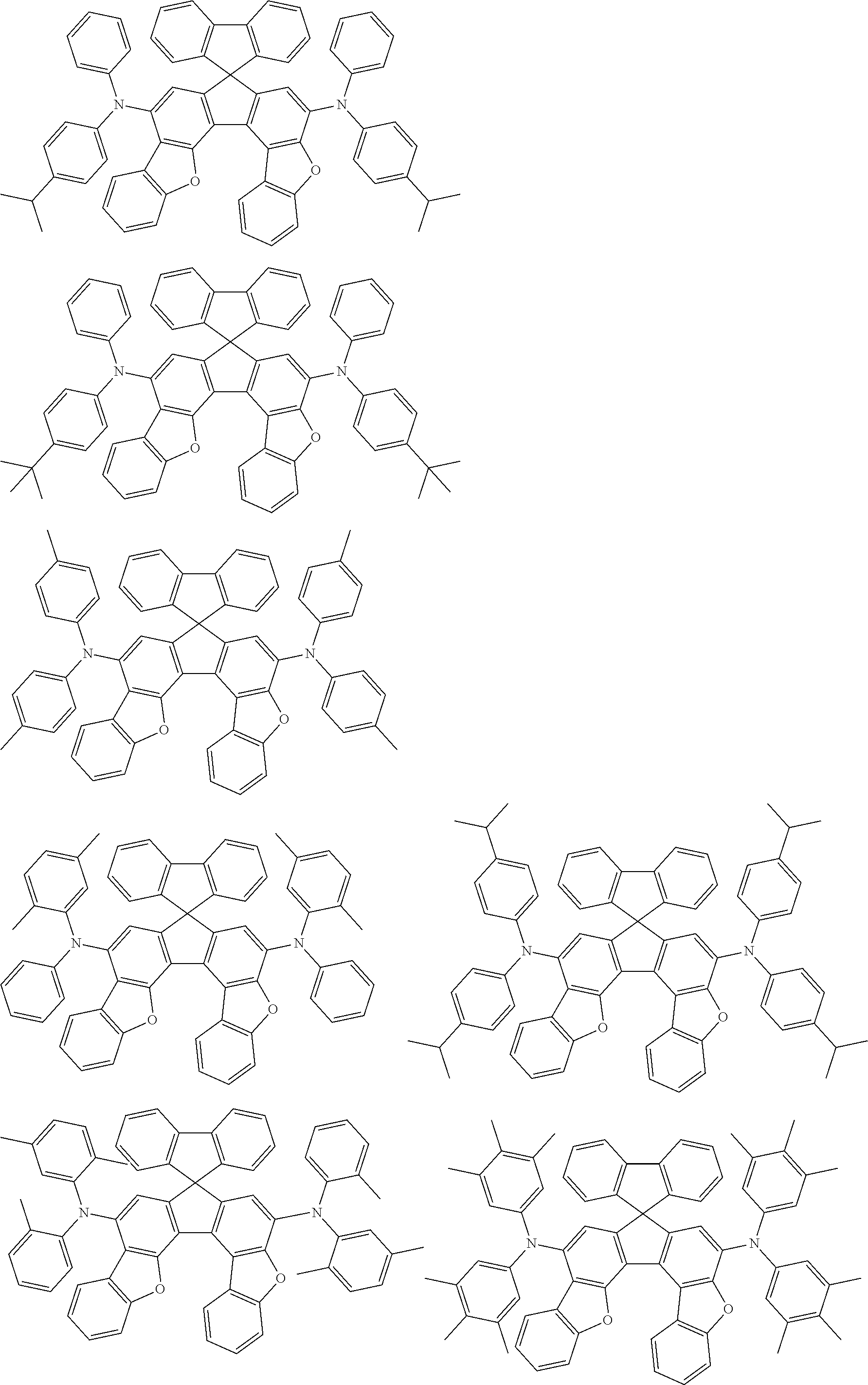

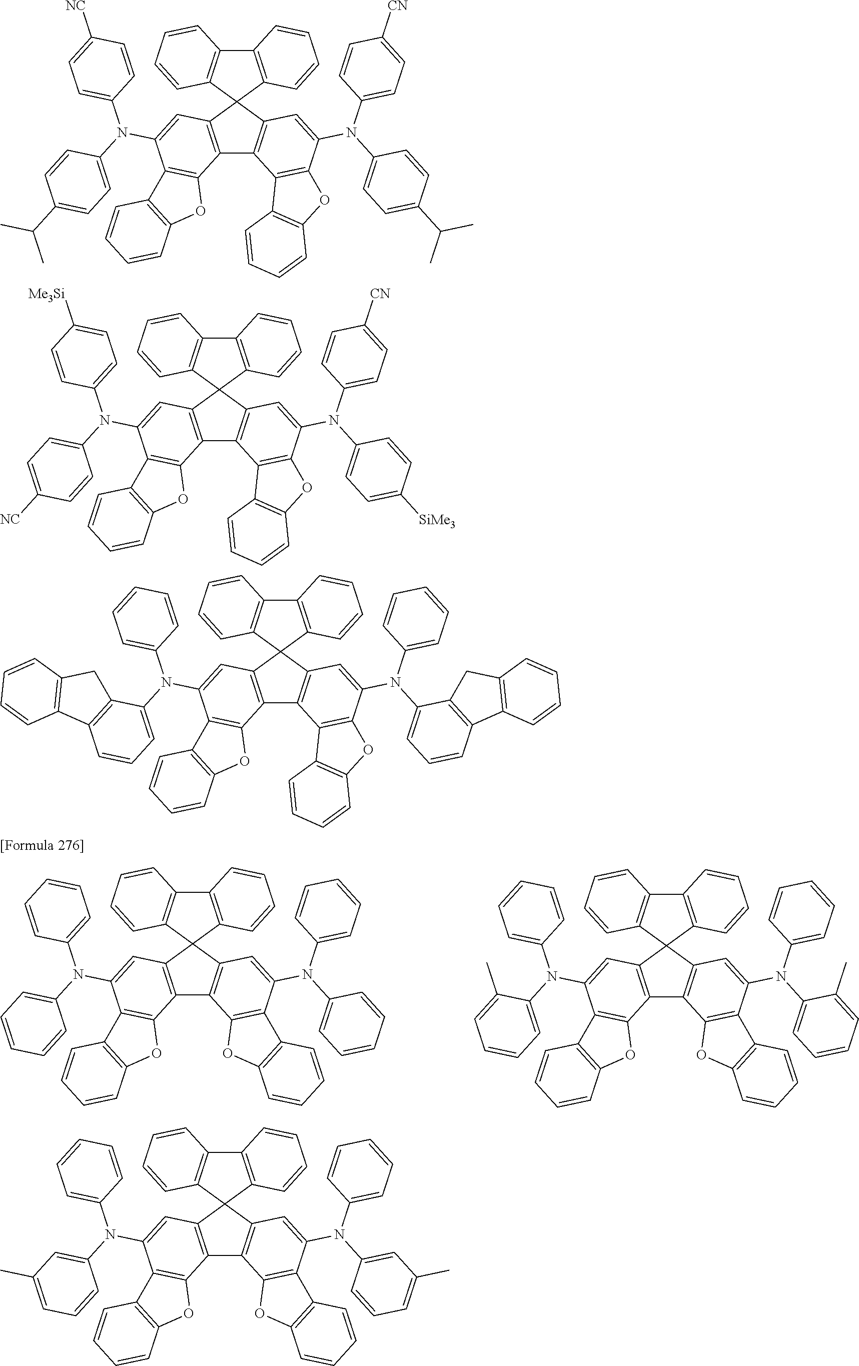

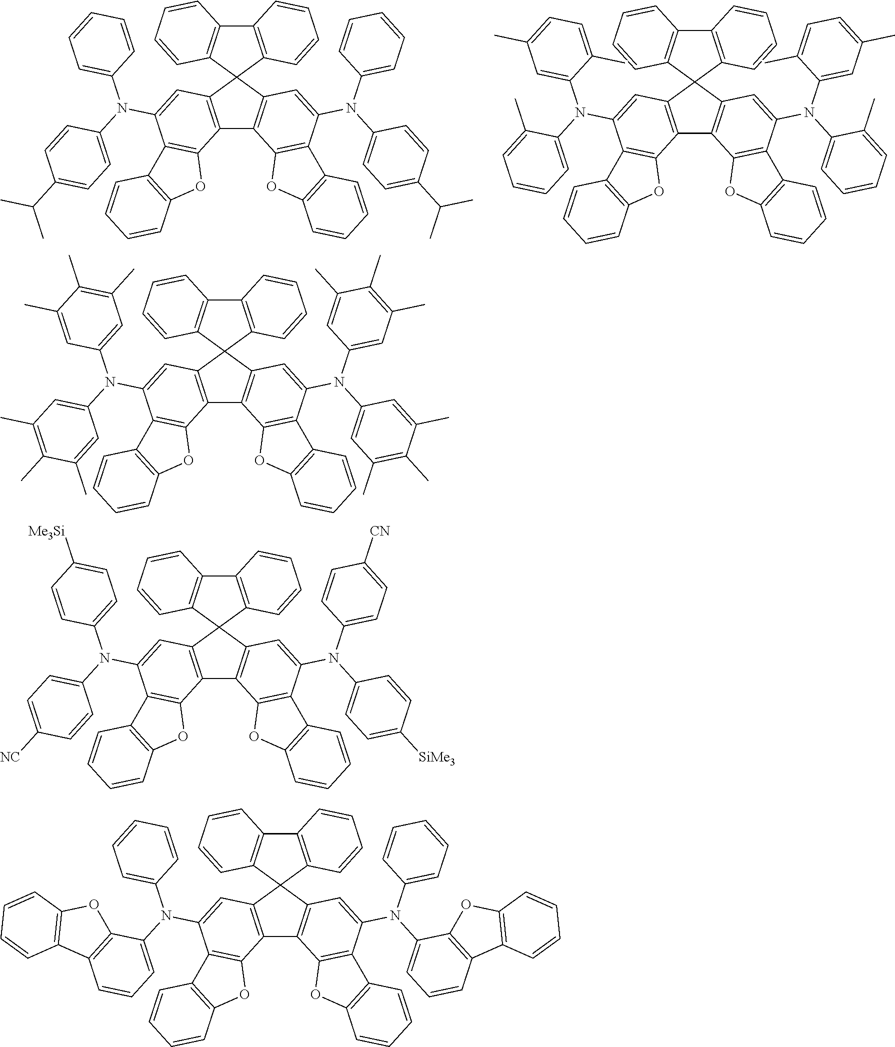

### Compound Represented by Formula (10)

The compound represented by the formula (10) will be described below.

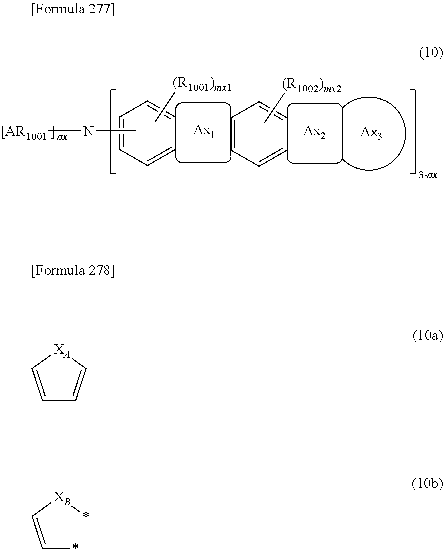

In the formula (10):

Ax₁ ring is a ring represented by the formula (10a) and fused with any position(s) of adjacent ring(s);

Ax₂ ring is a ring represented by the formula (10b) and fused with any position(s) of adjacent ring(s);

two * in the formula (10b) are bonded to any position of Ax₃ ring;

X_{A} and X_{B} are each independently C(R₁₀₀₃)(R₁₀₀₄), Si(R₁₀₀₅)(R₁₀₀₆), an oxygen atom or a sulfur atom;

Ax₃ ring is a substituted or unsubstituted aromatic hydrocarbon ring having 6 to 50 ring carbon atoms, or a substituted or unsubstituted heterocycle having 5 to 50 ring atoms;

Ar₁₀₀₁ is a substituted or unsubstituted aryl group having 6 to 50 ring carbon atoms, or a substituted or unsubstituted heterocyclic group having 5 to 50 ring atoms;

R₁₀₀₁ to R₁₀₀₆ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;

mx1 is 3, mx2 is 2;

a plurality of R₁₀₀₁ are mutually the same or different;

a plurality of R₁₀₀₂ are mutually the same or different;

ax is 0, 1, or 2;

when ax is 0 or 1, the structures enclosed by brackets indicated by “3-ax” are mutually the same or different; and

when ax is 2, a plurality of Ar₁₀₀₁ are mutually the same or different.

In an exemplary embodiment, Ar₁₀₀₁ is a substituted or unsubstituted aryl group having 6 to 50 ring carbon atoms.

In an exemplary embodiment, Ax₃ ring is a substituted or unsubstituted aromatic hydrocarbon ring having 6 to 50 ring carbon atoms, example of which is a substituted or unsubstituted benzene ring, a substituted or unsubstituted naphthalene ring, or a substituted or unsubstituted anthracene ring.

In an exemplary embodiment, R₁₀₀₃ and R₁₀₀₄ are each independently a substituted or unsubstituted alkyl group having 1 to 50 carbon atoms.

In an exemplary embodiment, ax is 1.

Specific examples of the compound represented by the formula (10) include compounds shown below.

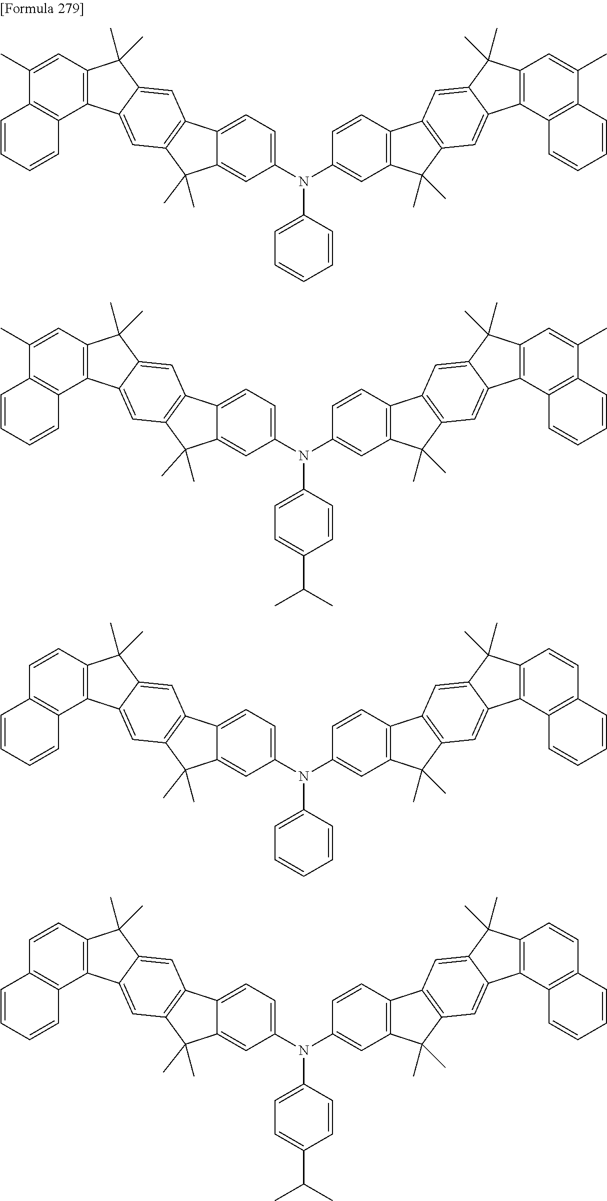

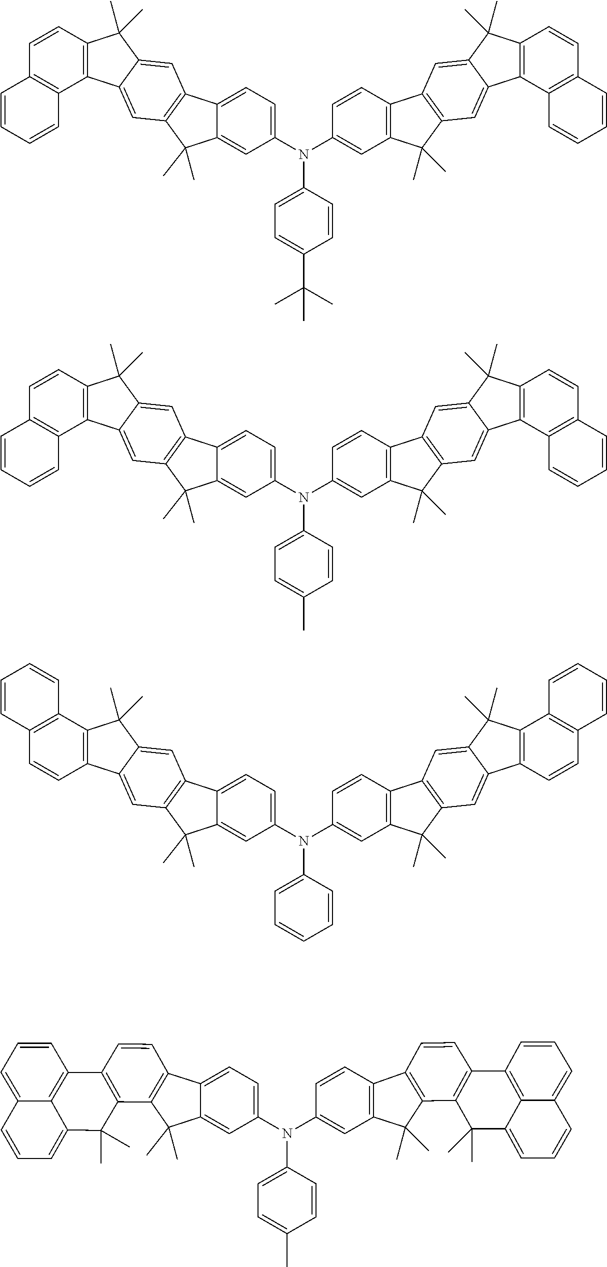 [SMILES: CC(C)(C)c1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; Cc1ccc(N(c2ccc3c(c2)C(C)(C)c2cc4c(cc2-3)C(C)(C)c2ccc3ccccc3c2-4)c2ccc3c(c2)C(C)(C)c2cc4c(cc2-3)C(C)(C)c2ccc3ccccc3c2-4)cc1 ; CC1(C)c2cc(N(c3ccccc3)c3ccc4c(c3)C(C)(C)c3cc5c(cc3-4)C(C)(C)c3c-5ccc4ccccc34)ccc2-c2cc3c(cc21)-c1ccc2ccccc2c1C3(C)C ; Cc1ccc(N(c2ccc3c(c2)C(C)(C)c2c-3ccc3c2C(C)(C)c2cccc4cccc-3c24)c2ccc3c(c2)C(C)(C)c2c-3ccc3c2C(C)(C)c2cccc4cccc-3c24)cc1]

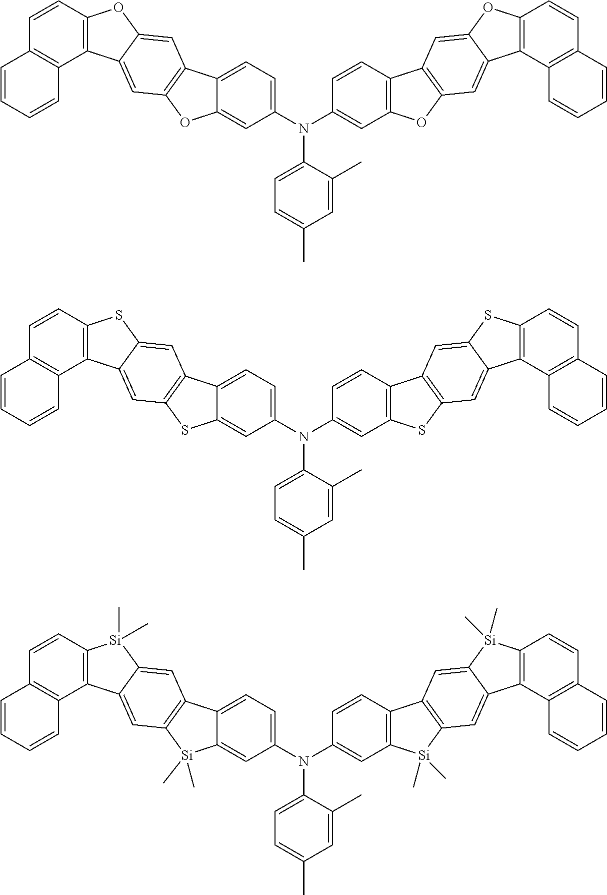 [SMILES: Cc1ccc(N(c2ccc3c(c2)oc2cc4c(cc23)oc2ccc3ccccc3c24)c2ccc3c(c2)oc2cc4c(cc23)oc2ccc3ccccc3c24)c(C)c1 ; Cc1ccc(N(c2ccc3c(c2)sc2cc4c(cc23)sc2ccc3ccccc3c24)c2ccc3c(c2)sc2cc4c(cc23)sc2ccc3ccccc3c24)c(C)c1 ; Cc1ccc(N(c2ccc3c(c2)[Si](C)(C)c2cc4c(cc2-3)[Si](C)(C)c2ccc3ccccc3c2-4)c2ccc3c(c2)[Si](C)(C)c2cc4c(cc2-3)[Si](C)(C)c2ccc3ccccc3c2-4)c(C)c1]

In an exemplary embodiment, the emitting layer contains, as at least one of the third compound or the fourth compound, at least one compound selected from the group consisting of the compound represented by the formula (4), the compound represented by the formula (5), the compound represented by the formula (7), the compound represented by the formula (8), the compound represented by the formula (9), and a compound represented by a formula (63a) below.

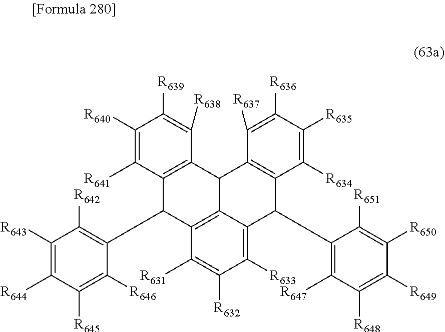

In the formula (63a):

R₆₃₁ is bonded with R₆₄₆ to form a substituted or unsubstituted heterocycle, or not bonded to form no substituted or unsubstituted heterocycle;

R₆₃₃ is bonded with R₆₄₇ to form a substituted or unsubstituted heterocycle, or not bonded to form no substituted or unsubstituted heterocycle;

R₆₃₄ is bonded with R₆₅₁ to form a substituted or unsubstituted heterocycle, or not bonded to form no substituted or unsubstituted heterocycle;

R₆₄₁ is bonded with R₆₄₂ to form a substituted or unsubstituted heterocycle, or not bonded to form no substituted or unsubstituted heterocycle;

at least one combination of adjacent two or more of R₆₃₁ to R₆₅₁ are mutually bonded to form a substituted or unsubstituted monocyclic ring, mutually bonded to form a substituted or unsubstituted fused ring, or not mutually bonded;

R₆₃₁ to R₆₅₁ not forming the substituted or unsubstituted heterocycle, not forming the monocyclic ring, and not forming the fused ring are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms; and

at least one of R₆₃₁ to R₆₅₁ not forming the substituted or unsubstituted heterocycle, not forming the monocyclic ring and not forming the fused ring are a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In an exemplary embodiment, the compound represented by the formula (4) is the compound represented by the formula (41-3), the formula (41-4) or the formula (41-5), the A1 ring in the formula (41-5) being a substituted or unsubstituted fused aromatic hydrocarbon ring having 10 to 50 ring carbon atoms, or a substituted or unsubstituted fused heterocycle having 8 to 50 ring atoms.

In an exemplary embodiment, the substituted or unsubstituted fused aromatic hydrocarbon ring having 10 to 50 ring carbon atoms in the formulae (41-3), (41-4) and (41-5) is a substituted or unsubstituted naphthalene ring, a substituted or unsubstituted anthracene ring, or a substituted or unsubstituted fluorene ring; and

the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, the substituted or unsubstituted fused aromatic hydrocarbon ring having 10 to 50 ring carbon atoms in the formula (41-3), (41-4) or (41-5) is a substituted or unsubstituted naphthalene ring, or a substituted or unsubstituted fluorene ring; and

the substituted or unsubstituted fused heterocycle having 8 to 50 ring atoms is a substituted or unsubstituted dibenzofuran ring, a substituted or unsubstituted carbazole ring, or a substituted or unsubstituted dibenzothiophene ring.

In an exemplary embodiment, the compound represented by the formula (4) is selected from the group consisting of a compound represented by a formula (461) below, a compound represented by a formula (462) below, a compound represented by a formula (463) below, a compound represented by a formula (464) below, a compound represented by a formula (465) below, a compound represented by a formula (466) below, and a compound represented by a formula (467) below.

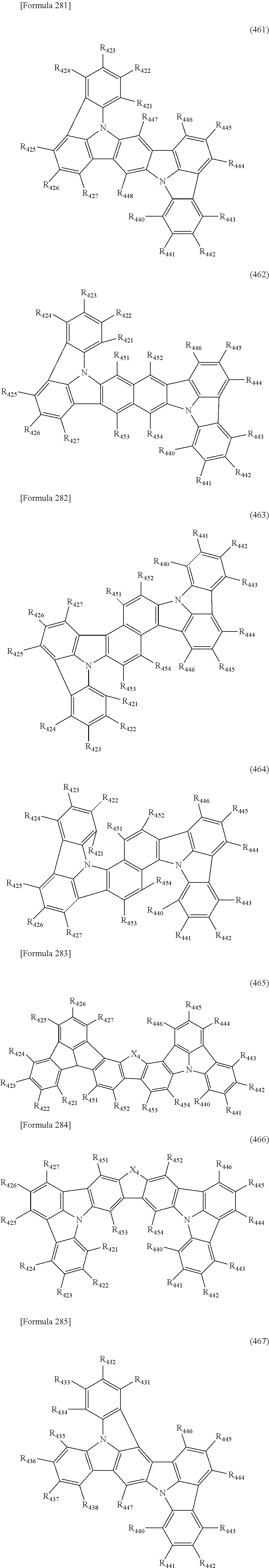

In the formulae (461) to (467):

at least one combination of adjacent two or more of moieties selected from R₄₂₁ to R₄₂₇, R₄₃₁ to R₄₃₆, R₄₄₀ to R₄₄₈, and R₄₅₁ to R₄₅₄ are mutually bonded to form a substituted or unsubstituted monocyclic ring, mutually bonded to form a substituted or unsubstituted fused ring, or not mutually bonded,

R₄₃₇, R₄₃₈, and R₄₂₁ to R₄₂₇, R₄₃₁ to R₄₃₆, R₄₄₀ to R₄₄₈ and R₄₅₁ to R₄₅₄ not forming the monocyclic ring and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;

X₄ is an oxygen atom, NR₈₀₁, or C(R₈₀₂)(R₈₀₃);

R₈₀₁, R₈₀₂, and R₈₀₃ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

a substituted or unsubstituted alkyl group having 1 to 50 carbon atoms, or a substituted or unsubstituted aryl group having 6 to 50 ring carbon atoms;

when a plurality of R₈₀₁ are present, the plurality of R₈₀₁ are mutually the same or different;

when a plurality of R₈₀₂ are present, the plurality of R₈₀₂ are mutually the same or different; and

when a plurality of R₈₀₃ are present, the plurality of R₈₀₃ are mutually the same or different.

In an exemplary embodiment, R₄₂₁ to R₄₂₇ and R₄₄₀ to R₄₄₈ are each independently a hydrogen atom, a substituted or unsubstituted aryl group having 6 to 50 ring carbon atoms, or a substituted or unsubstituted heterocyclic group having 5 to 50 ring atoms.

In an exemplary embodiment, R₄₂₁ to R₄₂₇ and R₄₄₀ to R₄₄₇ are each independently selected from the group consisting of a hydrogen atom, a substituted or unsubstituted aryl group having 6 to 18 ring carbon atoms, and a substituted or unsubstituted heterocyclic group having 5 to 18 ring atoms.

In an exemplary embodiment, the compound represented by the formula (41-3) is a compound represented by a formula (41-3-1) below.

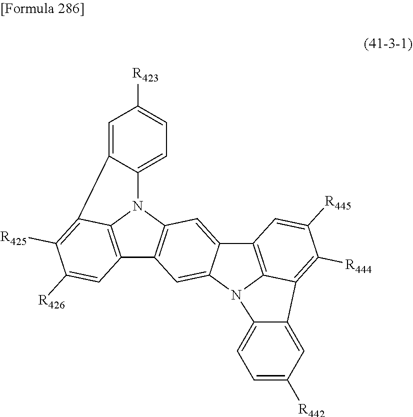

In the formula (41-3-1), R₄₂₃, R₄₂₅, R₄₂₆, R₄₄₂, R₄₄₄ and R₄₄₅ each independently represent the same as R₄₂₃, R₄₂₅, R₄₂₆, R₄₄₂, R₄₄₄ and R₄₄₅ in the formula (41-3).

In an exemplary embodiment, the compound represented by the formula (41-3) is represented by a formula (41-3-2) below.

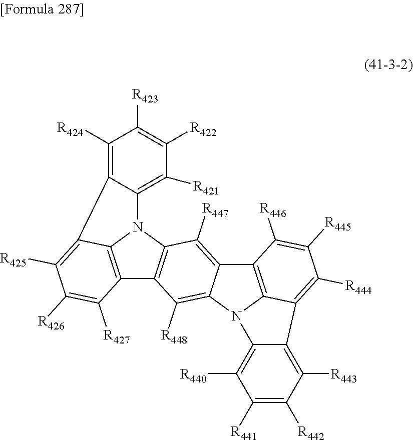

In the formula (41-3-2): R₄₂₁ to R₄₂₇ and R₄₄₀ to R₄₄₈ each independently represent the same as R₄₂₁ to R₄₂₇ and R₄₄₀ to R₄₄₈ in the formula (41-3); and at least one of R₄₂₁ to R₄₂₇ or R₄₄₀ to R₄₄₆ is a group represented by —N(R₉₀₆)(R₉₀₇).

In an exemplary embodiment, two of R₄₂₁ to R₄₂₇ and R₄₄₀ to R₄₄₆ in the formula (41-3-2) are groups represented by —N(R₉₀₆)(R₉₀₇).

In an exemplary embodiment, the compound represented by the formula (41-3-2) is a compound represented by a formula (41-3-3) below.

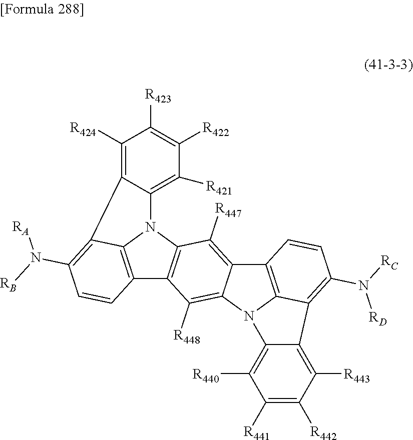

In the formula (41-3-3):

R₄₂₁ to R₄₂₄, R₄₄₀ to R₄₄₃, R₄₄₇, and R₄₄₈ each independently represent the same as R₄₂₁ to R₄₂₄, R₄₄₀ to R₄₄₃, R₄₄₇, and R₄₄₈ in the formula (41-3); and

R_{A}, R_{B}, R_{C}, and R_{D} are each independently a substituted or unsubstituted aryl group having 6 to 18 ring carbon atoms, or a substituted or unsubstituted heterocyclic group having 5 to 18 ring atoms.

In an exemplary embodiment, the compound represented by the formula (41-3-3) is a compound represented by a formula (41-3-4) below.

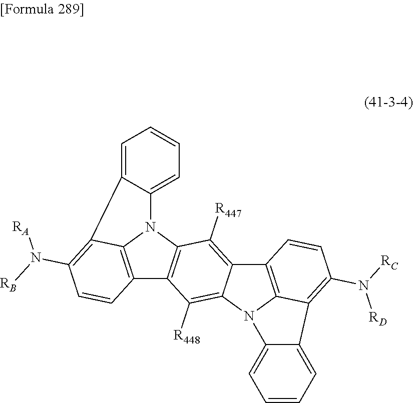

In the formula (41-3-4), R₄₄₇, R₄₄₈, R_{A}, R_{B}, R_{C} and R_{D} each independently represent the same as R₄₄₇, R₄₄₈, R_{A}, R_{B}, R_{C} and R_{D} in the formula (41-3-3).

In an exemplary embodiment, R_{A}, R_{B}, R_{C}, and R_{D} are each independently a substituted or unsubstituted aryl group having 6 to 18 ring carbon atoms.

In an exemplary embodiment, R_{A}, R_{B}, R_{C}, and R_{D} are each independently a substituted or unsubstituted phenyl group.

In an exemplary embodiment, R₄₄₇ and R₄₄₈ are each a hydrogen atom.

In an exemplary embodiment, a substituent “for the substituted or unsubstituted” group in each of the formulae is an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁ₐ)(R₉₀₂ₐ)(R₉₀₃ₐ), —O—(R₉₀₄ₐ), —S—(R₉₀₅ₐ), —N(R₉₀₆ₐ)(R₉₀₇ₐ), a halogen atom, a cyano group, a nitro group, an unsubstituted aryl group having 6 to 50 ring carbon atoms, or an unsubstituted heterocyclic group having 5 to 50 ring atoms;

R₉₀₁ₐ to R₉₀₇ₐ are each independently a hydrogen atom, an unsubstituted alkyl group having 1 to 50 carbon atoms, an unsubstituted aryl group having 6 to 50 ring carbon atoms, or an unsubstituted heterocyclic group having 5 to 50 ring atoms;

when two or more R₉₀₁ₐ are present, the two or more R₉₀₁ₐ are mutually the same or different;

when two or more R₉₀₂ₐ are present, the two or more R₉₀₂ₐ are mutually the same or different;

when two or more R₉₀₃ₐ are present, the two or more R₉₀₃ₐ are mutually the same or different;

when two or more R₉₀₄ₐ are present, the two or more R₉₀₄ₐ are mutually the same or different;

when two or more R₉₀₅ₐ are present, the two or more R₉₀₅ₐ are mutually the same or different;

when two or more R₉₀₆ₐ are present, the two or more R₉₀₆ₐ are mutually the same or different; and

when two or more R₉₀₇ₐ are present, the two or more R₉₀₇ₐ are mutually the same or different.

In an exemplary embodiment, a substituent “for the substituted or unsubstituted” group in each of the formulae is an unsubstituted alkyl group having 1 to 50 carbon atoms, an unsubstituted aryl group having 6 to 50 ring carbon atoms, or an unsubstituted heterocyclic group having 5 to 50 ring atoms.

In an exemplary embodiment, a substituent “for the substituted or unsubstituted” group in each of the formulae is an unsubstituted alkyl group having 1 to 18 carbon atoms, an unsubstituted aryl group having 6 to 18 ring carbon atoms, or an unsubstituted heterocyclic group having 5 to 18 ring atoms.

In the organic EL device according to the exemplary embodiment, it is preferable that the second emitting layer further contains a fourth compound that fluoresces, and the fourth compound is a compound that emits light having a main peak wavelength in a range from 430 nm to 480 nm.

In the organic EL device according to the exemplary embodiment, it is preferable that the first emitting layer further contains a third compound that fluoresces, and the third compound is a compound that emits light having a main peak wavelength in a range from 430 nm to 480 nm.

A measurement method of a main peak wavelength of the compound is as follows. A toluene solution of a measurement target compound at a concentration ranging from 10⁻⁶ mol/L to 10⁻⁵ mol/L is prepared and put in a quartz cell. An emission spectrum (ordinate axis: emission intensity, abscissa axis: wavelength) of the thus-obtained sample is measured at a normal temperature (300K). The emission spectrum is measurable using a spectrophotometer (machine name: F-7000) manufactured by Hitachi High-Tech Science Corporation. It should be noted that the machine for measuring the emission spectrum is not limited to the machine used herein.

A peak wavelength of the emission spectrum exhibiting the maximum luminous intensity is defined as a main peak wavelength. It should be noted that the main peak wavelength is sometimes referred to as a fluorescence main peak wavelength (FL-peak) herein.

When the first emitting layer of the organic EL device according to the exemplary embodiment contains the first compound and the third compound, the first compound is preferably a host material (sometimes referred to as a matrix material) and the third compound is preferably a dopant material (sometimes referred to as a guest material, emitter, or luminescent material).

When the first emitting layer of the organic EL device according to the exemplary embodiment contains the first compound and the third compound, a singlet energy S₁(H1) of the first compound and a singlet energy S₁(D3) of the third compound preferably satisfy a relationship of a numerical formula (Numerical Formula 1) below.

S₁(H1)>S₁(D3) (Numerical Formula 1)

When the second emitting layer of the organic EL device according to the exemplary embodiment contains the second compound and the fourth compound, the second compound is preferably a host material (sometimes referred to as a matrix material) and the fourth compound is preferably a dopant material (sometimes referred to as a guest material, emitter, or luminescent material).

When the second emitting layer of the organic EL device according to the exemplary embodiment contains the second compound and the fourth compound, a singlet energy S₁(H2) of the second compound and a singlet energy S₁(D4) of the fourth compound preferably satisfy a relationship of a numerical formula (Numerical Formula 2) below.

S₁(H2)>S₁(D4) (Numerical Formula 2)

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent was drawn to the fall of the absorption spectrum close to the long-wavelength region, and a wavelength value λedge (nm) at an intersection of the tangent and the abscissa axis was assigned to a conversion equation (F2) below to calculate the singlet energy.

S₁ [eV]=1239.85/λedge Conversion Equation (F2)

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve fell (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the organic EL device according to the exemplary embodiment, an electron mobility μH1 of the first compound and an electron mobility μH2 of the second compound also preferably satisfy a relationship of a numerical formula (Numerical Formula 3) below.

μH2>μH1 (Numerical Formula 3)

When the first compound and the second compound satisfy the relationship of the numerical formula (Numerical Formula 3), a recombination ability of holes and electrons in the first emitting layer is improved.

The electron mobility can be measured according to impedance spectroscopy.

A measurement target layer having a thickness in a range from 100 nm to 200 nm is held between the anode and the cathode, to which a small alternating voltage of 100 mV or less is applied while a bias DC voltage is applied. A value of an alternating current (absolute value and phase) which flows at this timc is measured. This measurement is performed while changing a frequency of the alternating voltage, and complex impedance (Z) is calculated from the current value and the voltage value. A frequency dependency of the imaginary part (ImM) of the modulus M=iωZ (i: imaginary unit, ω: angular frequency) is obtained. The reciprocal number of a frequency ω at which the ImM becomes the maximum is defined as a response time of electrons carried in the measurement target layer. The electron mobility is calculated by the following equation.

Electron Mobility=(Film Thickness of Measurement Target Layer)²/(Response Time-Voltage)

The first emitting layer and the second emitting layer preferably do not contain a phosphorescent material (dopant material).

The first emitting layer and the second emitting layer preferably do not contain a heavy metal complex and a phosphorescent rare earth metal complex. Examples of the heavy-metal complex herein include iridium complex, osmium complex, and platinum complex.

Further, the first emitting layer and the second emitting layer also preferably do not contain a metal complex.

### Film Thickness of Emitting Layer

A film thickness of the emitting layer of the organic EL device according to the exemplary embodiment is preferably in a range from 5 nm to 50 nm, more preferably in a range from 7 nm to 50 nm, further preferably in a range from 10 nm to 50 nm. When the film thickness of the emitting layer is 5 nm or more, the emitting layer is easily formable and chromaticity is easily adjustable. When the film thickness of the emitting layer is 50 nm or less, a rise in the drive voltage is easily reducible.

### Content Ratios of Compounds in Emitting Layer

When the first emitting layer contains the first compound and the third compound, a content ratio of each of the first compound and the third compound in the first emitting layer preferably falls, for instance, within a range below.

The content ratio of the first compound is preferably in a range from 80 mass % to 99 mass %, more preferably in a range from 90 mass % to 99 mass %, further preferably in a range from 95 mass % to 99 mass %.

The content ratio of the third compound is preferably in a range from 1 mass % to 10 mass %, more preferably in a range from 1 mass % to 7 mass %, further preferably in a range from 1 mass % to 5 mass %.

The upper limit of the total of the content ratios of the first compound and the third compound in the first emitting layer is 100 mass %.

It is not excluded that the first emitting layer of the exemplary embodiment further contains a material(s) other than the first and third compounds.

The first emitting layer may include a single type of the first compound or may include two or more types of the first compound. The first emitting layer may include a single type of the third compound or may include two or more types of the third compound.

When the second emitting layer contains the second compound and the fourth compound, a content ratio of each of the second compound and the fourth compound in the second emitting layer preferably falls, for instance, within a range below.

The content ratio of the second compound is preferably in a range from 80 mass % to 99 mass %, more preferably in a range from 90 mass % to 99 mass %, further preferably in a range from 95 mass % to 99 mass %.

The content ratio of the fourth compound is preferably in a range from 1 mass % to 10 mass %, more preferably in a range from 1 mass % to 7 mass %, further preferably in a range from 1 mass % to 5 mass %.

The upper limit of the total of the content ratios of the second compound and the fourth compound in the second emitting layer is 100 mass %.

It is not excluded that the second emitting layer of the exemplary embodiment further contains a material(s) other than the second and fourth compounds.

The second emitting layer may include a single type of the second compound or may include two or more types of the second compound. The second emitting layer may include a single type of the fourth compound or may include two or more types of the fourth compound.

An arrangement of the organic EL device 1 will be further described. It should be noted that the reference numerals will be sometimes omitted below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AILi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AILi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(Vs) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/Vs or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A film thickness of each of the organic layers of the organic EL device in the exemplary embodiment is not limited unless otherwise specified in the above. In general, the thickness preferably ranges from several nanometers to 1 μm because excessively small film thickness is likely to cause defects (e.g. pin holes) and excessively large thickness leads to the necessity of applying high voltage and consequent reduction in efficiency.

According to the exemplary embodiment, an organic electroluminescence device with improved luminous efficiency can be provided.

In the organic EL device according to the exemplary embodiment, the first emitting layer containing the first host material in a form of the first compound represented by the formula (1) or the like and the second emitting layer containing the second host material in a form of the second compound represented by the formula (2) or the like are in direct contact with each other. By thus layering the first emitting layer and the second emitting layer, the generated singlet exitons and the triplet exitons can be efficiently used and, consequently, the luminous efficiency of the organic EL device can be improved.

### Second Exemplary Embodiment

### Electronic Device

An electronic device according to a second exemplary embodiment is installed with any one of the organic EL devices according to the above exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited by the above-described exemplary embodiments but includes any modification and improvement as long as such modification and improvement are compatible with the invention.

For instance, the number of emitting layers is not limited to two, and more than two emitting layers may be provided and laminated with each other. When the organic EL device includes more than two emitting layers, it is only necessary that at least two of the emitting layers should satisfy the requirements mentioned in the above exemplary embodiment. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, excitons or combinations thereof.

For instance, when the blocking layer is provided in contact with the side of the emitting layer close to the cathode, the blocking layer permits transport of electrons and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### EXAMPLES

The invention will be described in further detail with reference to Examples. It should be noted that the scope of the invention is by no means limited to Examples.

### Compounds

Structures of compounds used for manufacturing organic EL devices in Examples or Reference Examples are shown below.

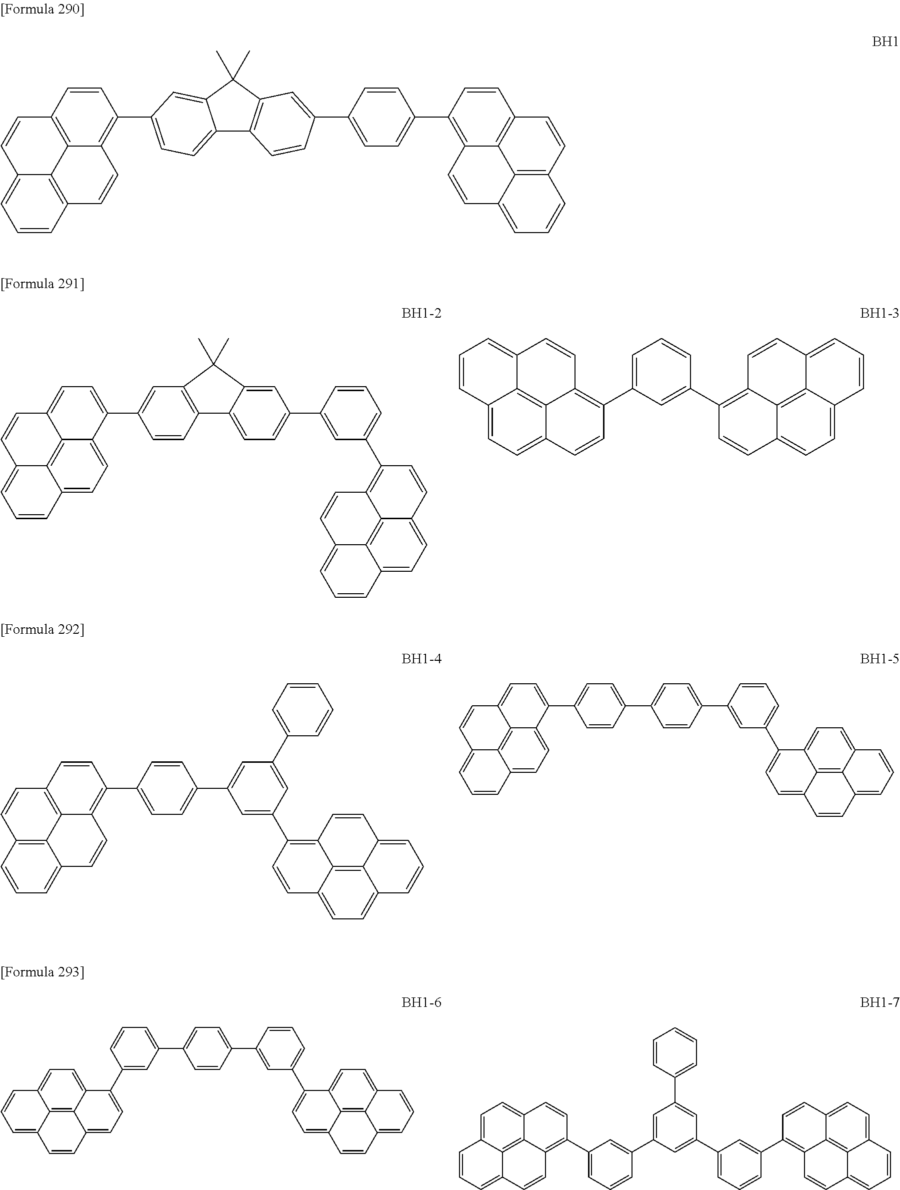

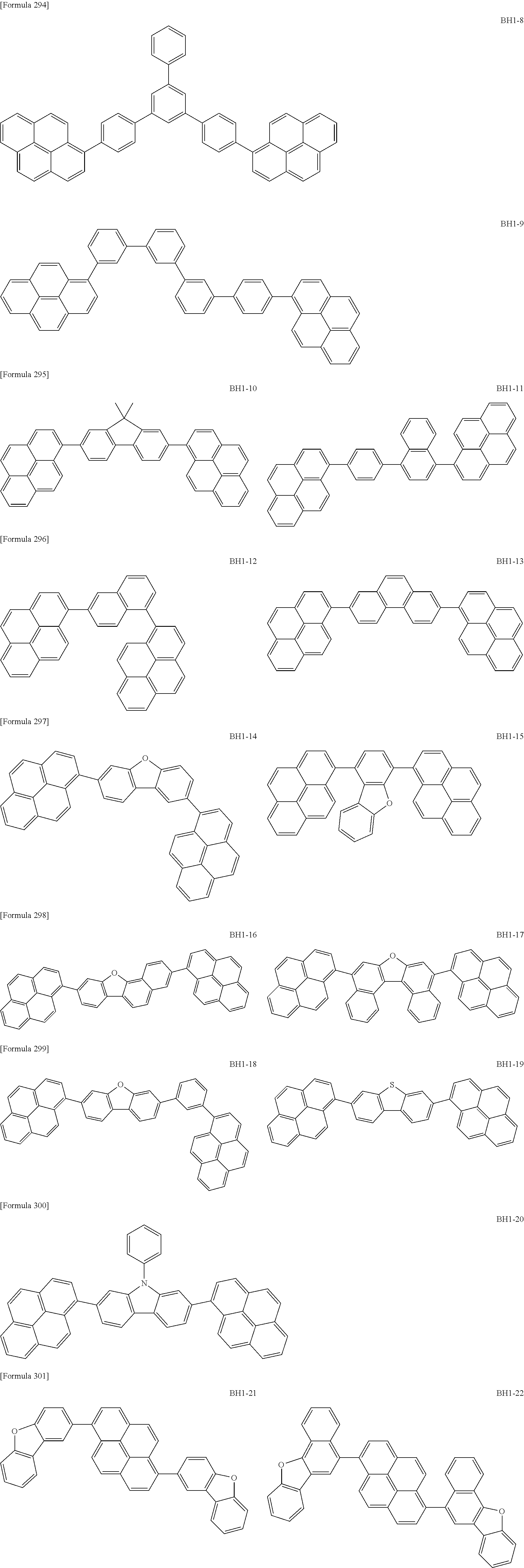

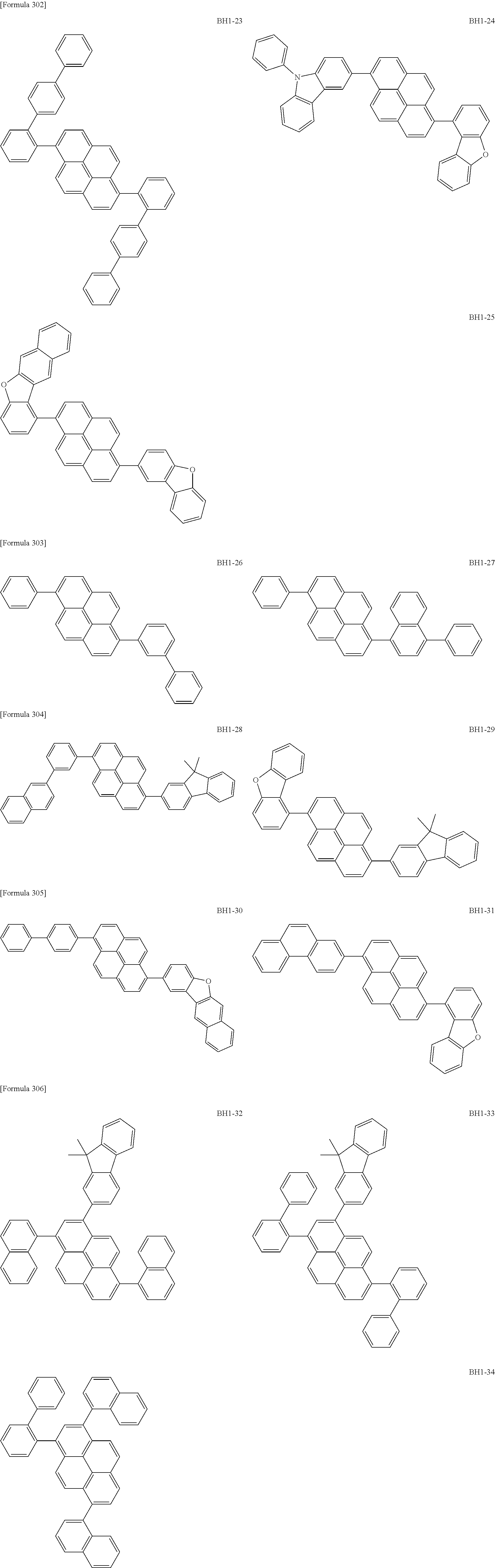

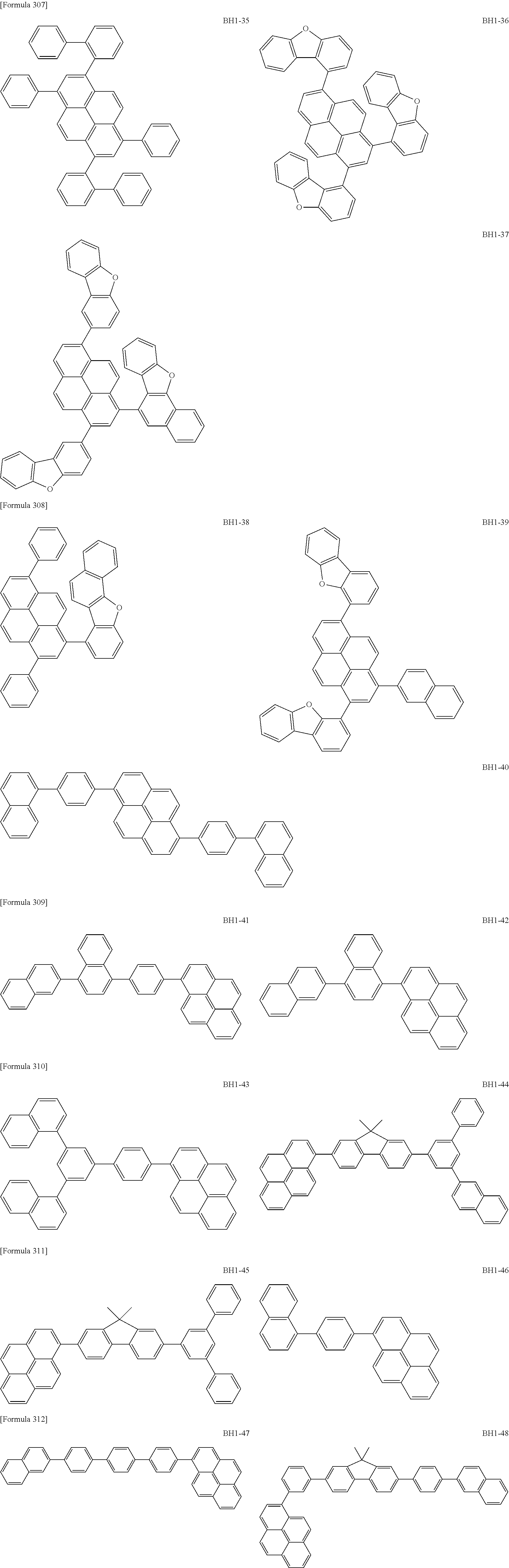

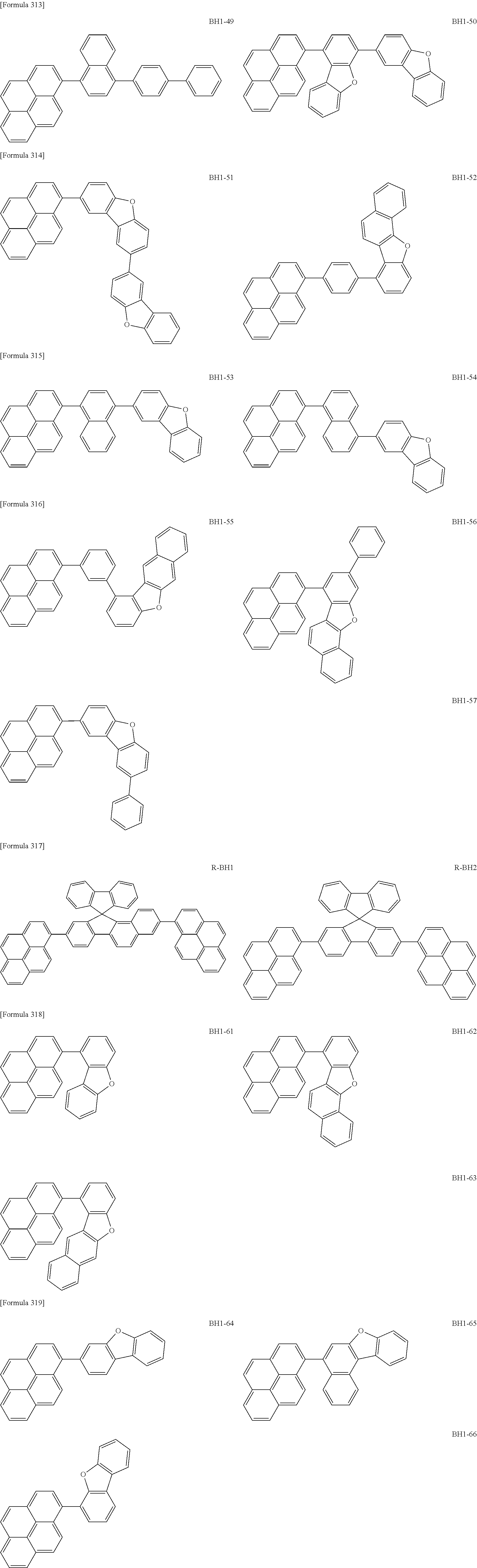

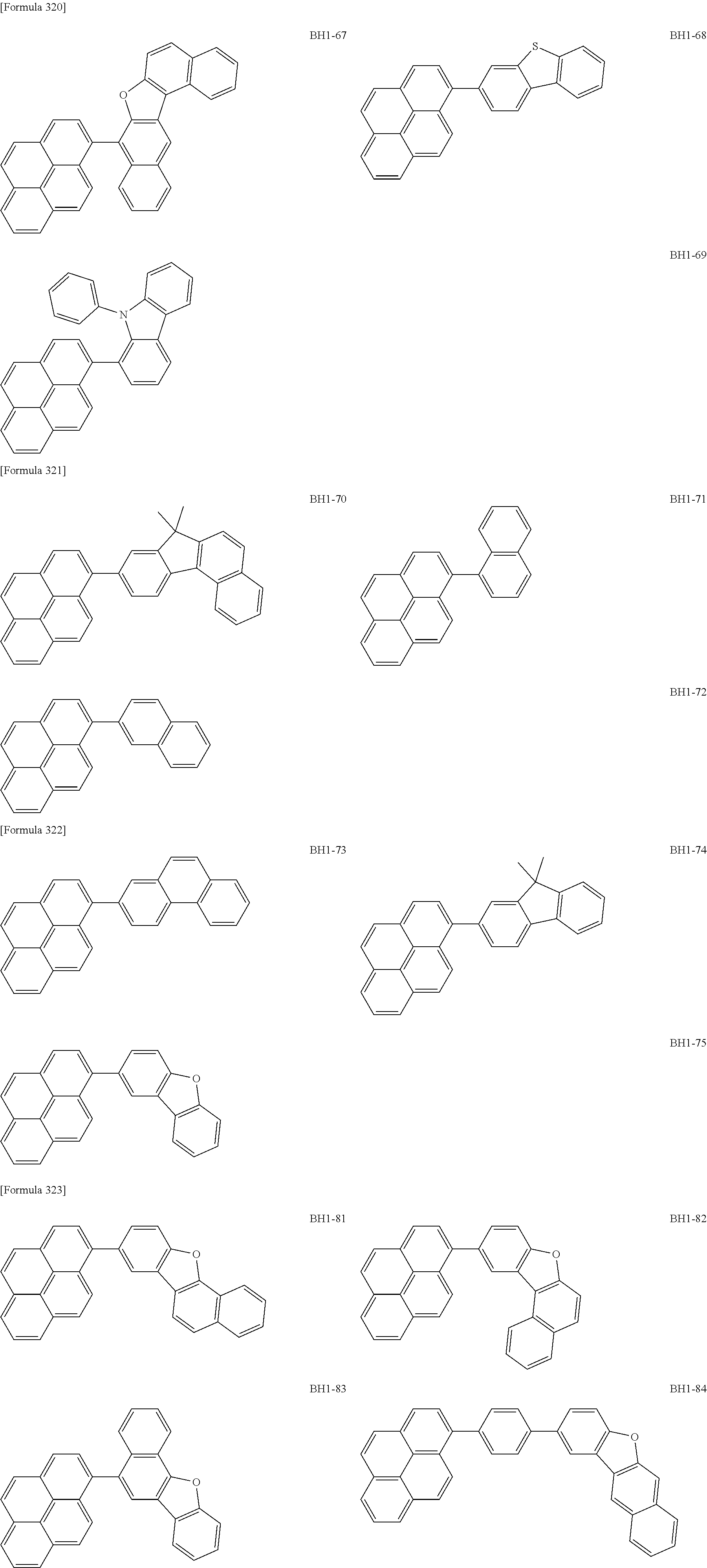

A structure of a compound used for manufacturing an organic EL device in Comparative Example is shown below.

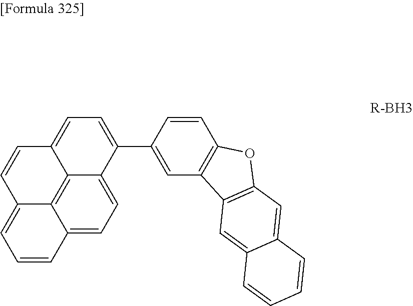 [SMILES: c1ccc2cc3c(cc2c1)oc1ccc(-c2ccc4ccc5cccc6ccc2c4c56)cc13]

Structures of compounds used for manufacturing organic EL devices in Examples or Reference Examples are shown below.

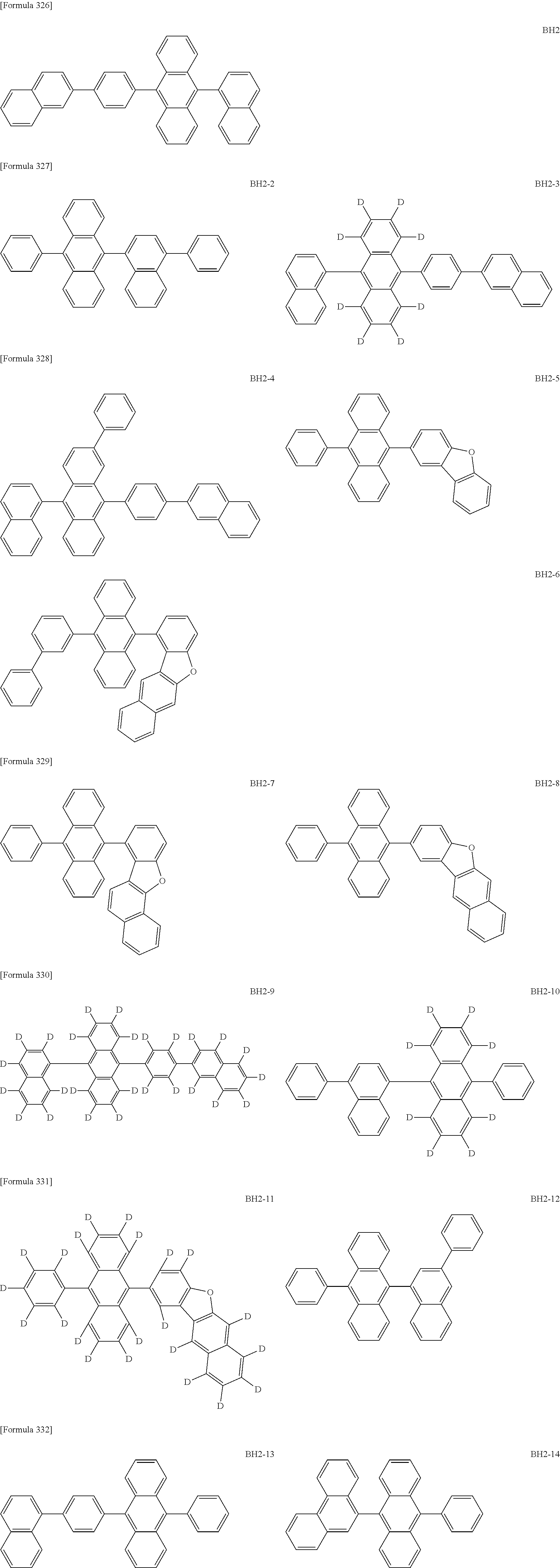

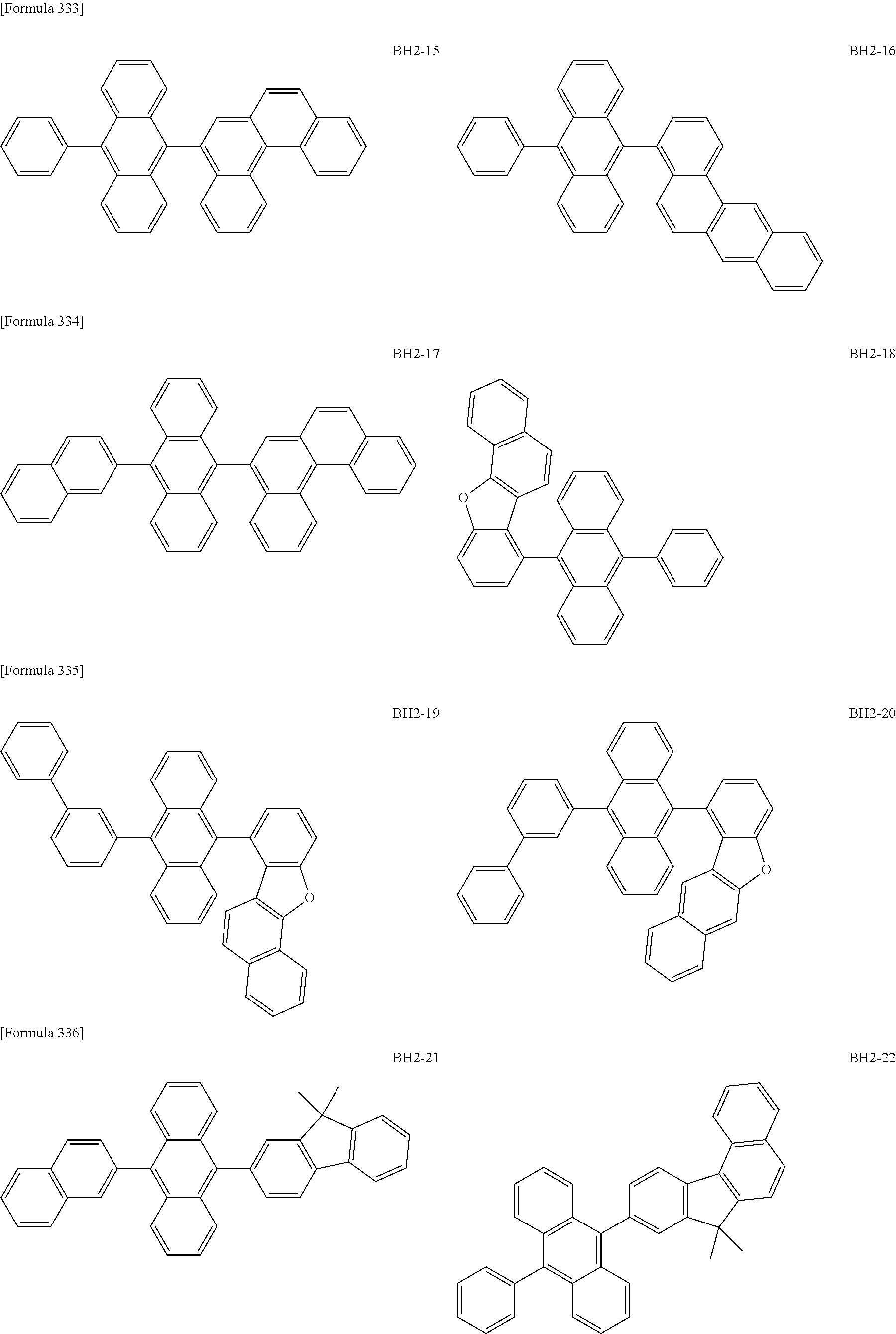

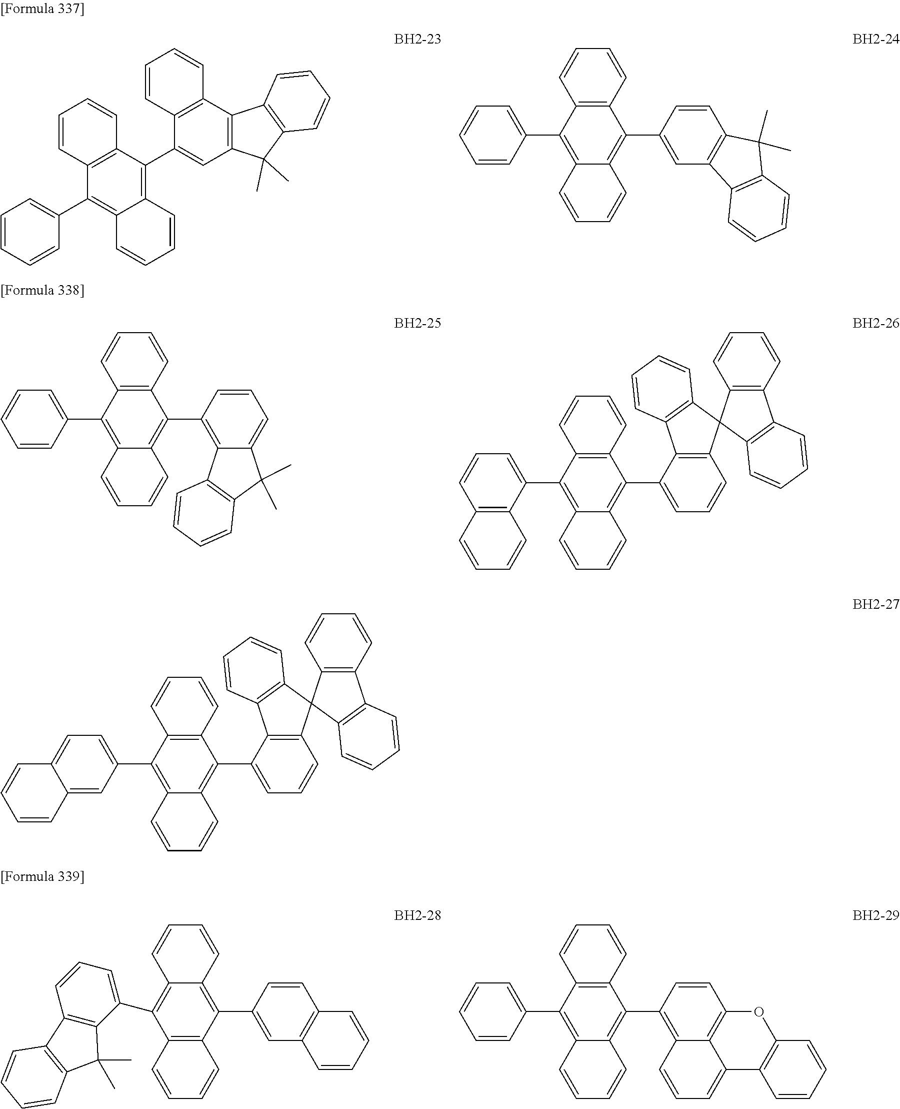

Structures of other compounds used for manufacturing organic EL devices in Examples, Comparative Examples, Reference Examples and Comparative Reference Examples are shown below.

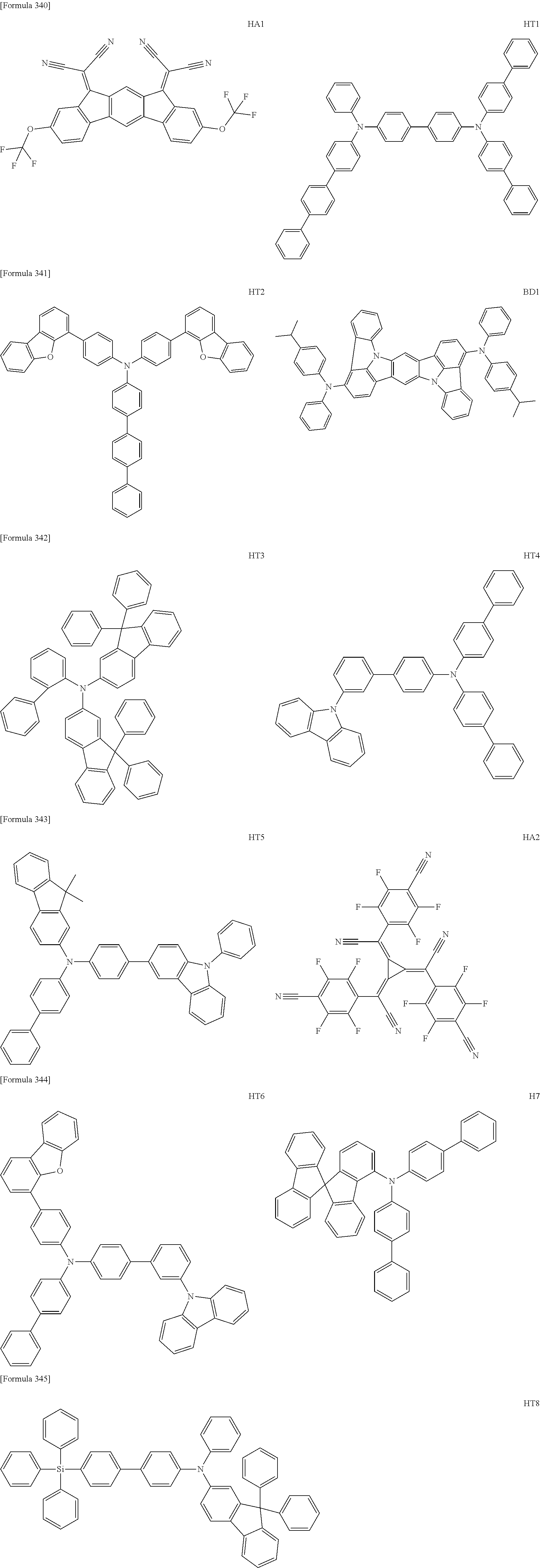

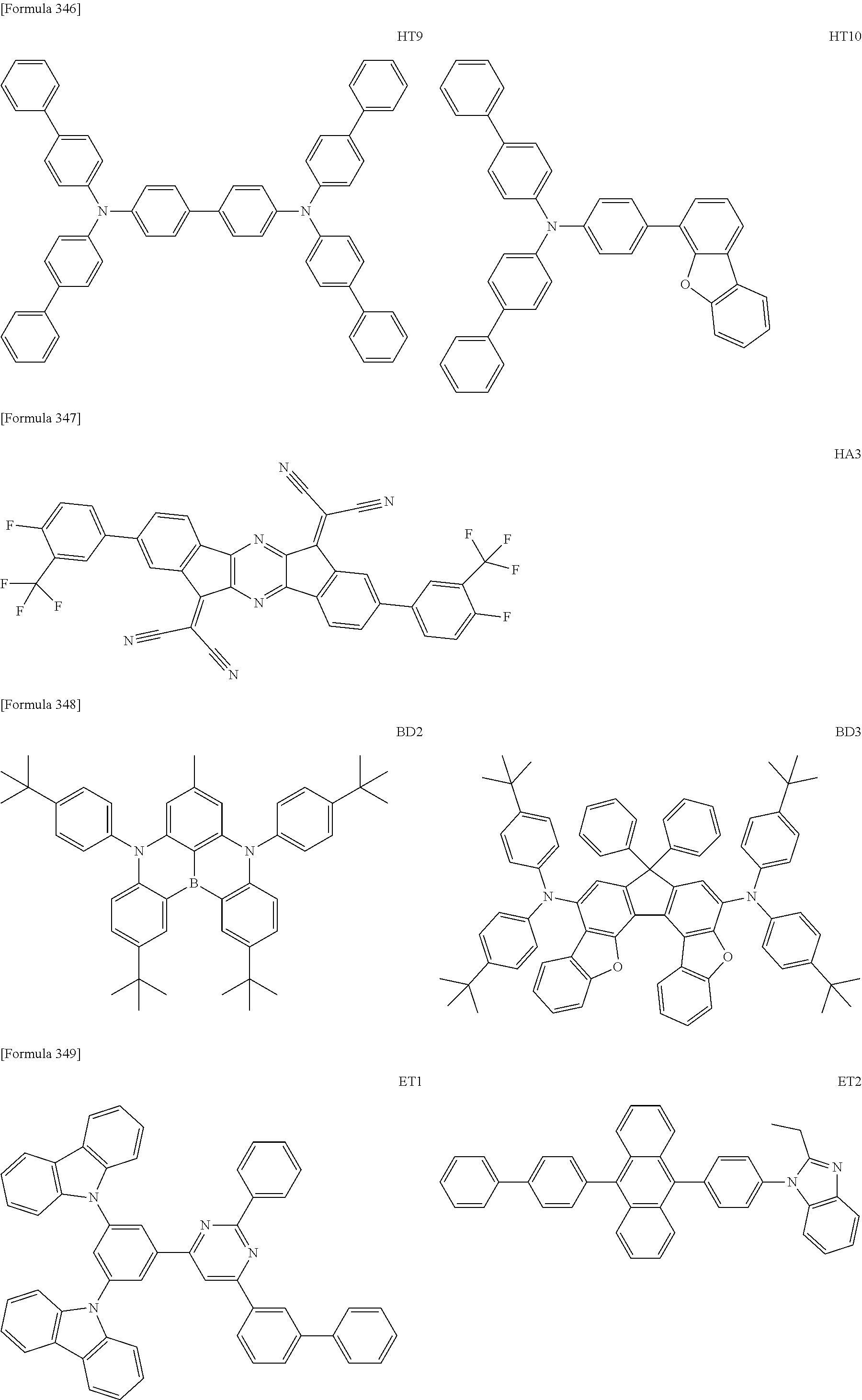

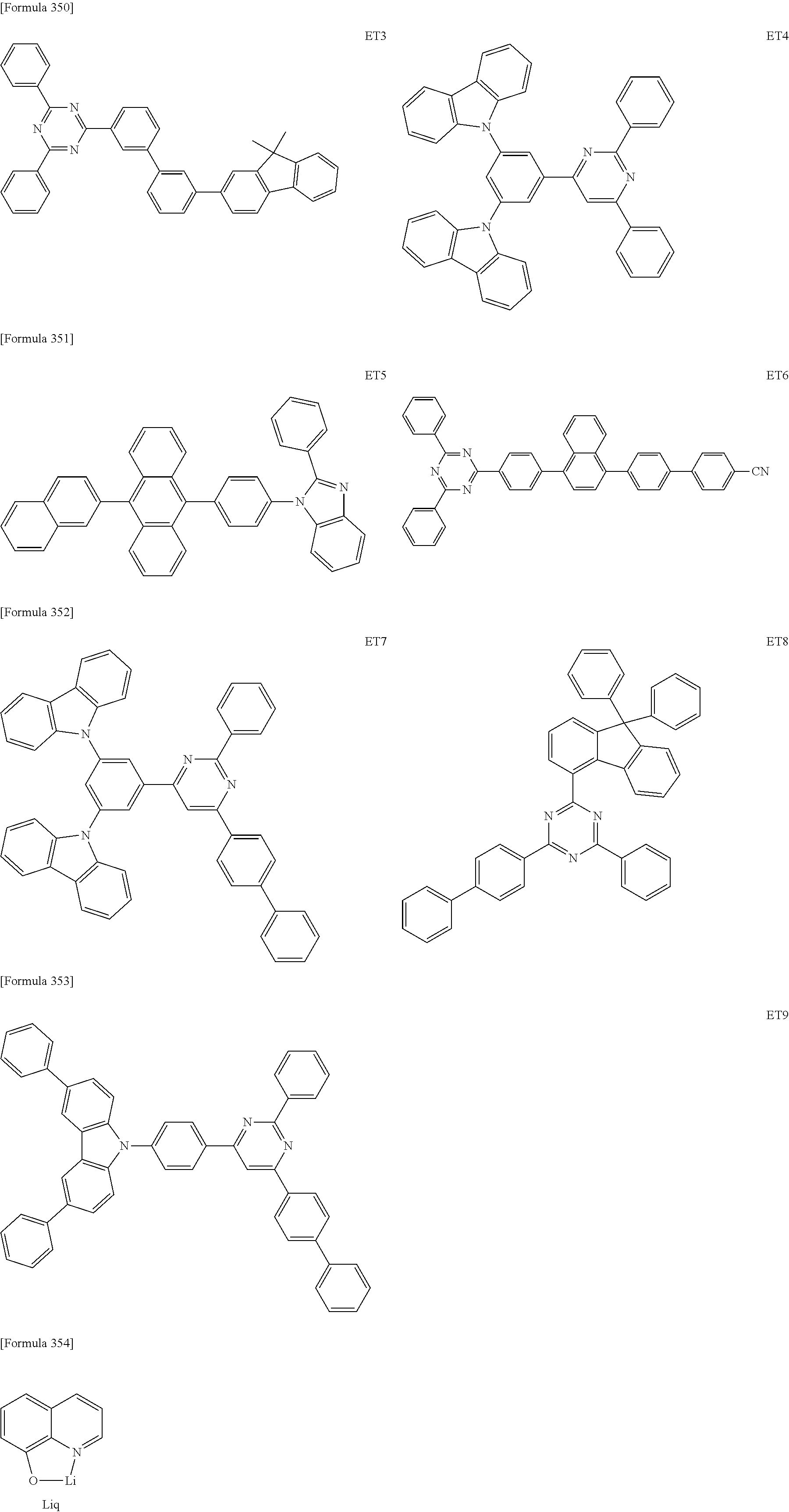

### Manufacture 1 of Organic EL Device

Organic EL devices were manufactured and evaluated as follows.

### Reference Example 1

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

The compound BH1 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 1 is roughly shown as follows.

### ITO(130)/HA1(5)/HT1(80)/HT2(10)/BH1:BD1(5.98%:2%)/BH2:BD1(20.98%:2%)/ET1(10)/ET2(15)/LiF(1)/Al(80)

The numerals in parentheses represent film thickness (unit: nm).

The numerals (98%:2%) represented by percentage in the same parentheses indicate a ratio (mass %) between the host material (compound BH1 or BH2) and the compound BD1 in the first emitting layer or the second emitting layer. Similar notations apply to the description below.

### Comparative Reference Example 1

An organic EL device in Comparative Reference Example 1 was manufactured in the same manner as that of Reference Example 1 except that a 25-nm-thick first emitting layer was formed as the emitting layer, and the first electron transporting layer was formed on the first emitting layer without forming the second emitting layer, as shown in Table 1.

### Comparative Reference Example 2

An organic EL device in Comparative Reference Example 2 was manufactured in the same manner as that of Reference Example 1 except that a 25-nm-thick second emitting layer was formed as the emitting layer on the second hole transporting layer without forming the first emitting layer, as shown in Table 1.

### Evaluation of Organic EL Devices

The organic EL devices manufactured in Examples, Comparative Examples, Reference Examples, and Comparative Reference Examples were evaluated as follows. Evaluation results are shown in Tables 1 to 46.

Herein, evaluation results of some Examples, some Comparative Examples, some Reference Examples, and some Comparative Reference Examples are shown in a plurality of Tables.

### External Quantum Efficiency EQE

Voltage was applied on the organic EL devices so that a current density was 10 mA/cm², where spectral radiance spectrum was measured by a spectroradiometer (CS-2000 manufactured by Konica Minolta, Inc.). The external quantum efficiency EQE (unit: %) was calculated based on the obtained spectral-radiance spectra, assuming that the spectra was provided under a Lambertian radiation.

### Lifetime LT90

Voltage was applied on the resultant organic EL devices so that a current density was 50 mA/cm², where a time (LT90 (unit: hr)) elapsed before a luminance intensity was reduced to 90% of the initial luminance intensity was measured.

### Lifetime LT95

Voltage was applied on the resultant organic EL devices so that a current density was 50 mA/cm², where a time (LT95 (unit: hr)) elapsed before a luminance intensity was reduced to 95% of the initial luminance intensity was measured.

### Main Peak Wavelength λp When Device is Driven

Voltage was applied on the organic EL devices so that a current density of the organic EL device was 10 mA/cm², where spectral radiance spectrum was measured by a spectroradiometer CS-2000 (manufactured by Konica Minolta, Inc.). The main peak wavelength λp (unit: nm) was calculated based on the obtained spectral radiance spectrum.

### Drive Voltage

The voltage (unit: V) when electric current was applied between the anode and the cathode so that the current density was 10 mA/cm² was measured.

### CIE1931 Chromaticity

Voltage was applied on the organic EL devices so that a current density was 10 mA/cm², where spectral radiance spectrum was measured by a spectroradiometer (CS-2000 manufactured by Konica Minolta, Inc.). Chromaticities CIEy for the organic EL devices in Example 145 and Comparative Example 118 were each calculated based on the obtained spectral radiance spectra.

TABLE 1

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | EQE | LT90 | λp |
| | Compound | Compound | [nm] | Compound | Compound | [nm] | [%] | [hr] | [nm] |
| Ref. Ex. 1 | BH1 | BD1 | 5 | BH2 | BD1 | 20 | 10.6 | 600 | 461 |
| Comp. Ref. Ex. 1 | BH1 | BD1 | 25 | — | — | — | 7.6 | 360 | 462 |
| Comp. Ref. Ex. 2 | — | — | — | BH2 | BD1 | 25 | 9.9 | 363 | 460 |

As shown in Table 1, the organic EL device according to Reference Example 1, in which the first emitting layer containing the first host material in a form of the first compound was in direct contact with the second emitting layer containing the second host material in a form of the second compound, emitted light at a higher luminous efficiency than the organic EL devices according to Comparative Reference Examples 1 and 2 including only one of the emitting layers. Moreover, the organic EL device according to Reference Example 1 exhibited longer lifetime than those of the organic EL devices according to Comparative Reference Examples 1 and 2.

### Reference Examples 2 to 20

Organic EL devices in Reference Examples 2 to 20 were manufactured in the same manner as in Reference Example 1 except that the compound BH1 (first host material) in the first emitting layer was replaced by the first compound shown in Table 2.

### Comparative Reference Examples 3 to 21

Organic EL devices in Comparative Reference Examples 3 to 21 were manufactured in the same manner as that of Comparative Reference Example 1 except that the compound BH1 (first host material) in the first emitting layer was replaced by the first compound listed in Table 3.

TABLE 2

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 1 | BH1 | BD1 | 5 | BH2 | BD1 | 20 | 3.47 | 10.6 | 255 |
| Ref. Ex. 2 | BH1-2 | BD1 | 5 | BH2 | BD1 | 20 | 3.47 | 10.2 | 205 |
| Ref. Ex. 3 | BH1-3 | BD1 | 5 | BH2 | BD1 | 20 | 3.56 | 10.5 | 268 |
| Ref. Ex. 4 | BH1-4 | BD1 | 5 | BH2 | BD1 | 20 | 3.56 | 10.7 | 222 |
| Ref. Ex. 5 | BH1-5 | BD1 | 5 | BH2 | BD1 | 20 | 3.64 | 10.7 | 251 |
| Ref. Ex. 6 | BH1-6 | BD1 | 5 | BH2 | BD1 | 20 | 3.65 | 10.6 | 224 |
| Ref. Ex. 7 | BH1-7 | BD1 | 5 | BH2 | BD1 | 20 | 3.63 | 10.4 | 239 |
| Ref. Ex. 8 | BH1-8 | BD1 | 5 | BH2 | BD1 | 20 | 3.62 | 10.4 | 224 |
| Ref. Ex. 9 | BH1-9 | BD1 | 5 | BH2 | BD1 | 20 | 3.70 | 10.8 | 249 |
| Ref. Ex. 10 | BH1-10 | BD1 | 5 | BH2 | BD1 | 20 | 3.34 | 10.4 | 216 |
| Ref. Ex. 11 | BH1-11 | BD1 | 5 | BH2 | BD1 | 20 | 3.48 | 10.8 | 275 |
| Ref. Ex. 12 | BH1-12 | BD1 | 5 | BH2 | BD1 | 20 | 3.39 | 10.6 | 212 |
| Ref. Ex. 13 | BH1-13 | BD1 | 5 | BH2 | BD1 | 20 | 3.51 | 10.6 | 231 |
| Ref. Ex. 14 | BH1-14 | BD1 | 5 | BH2 | BD1 | 20 | 3.36 | 10.4 | 198 |
| Ref. Ex. 15 | BH1-15 | BD1 | 5 | BH2 | BD1 | 20 | 3.43 | 10.5 | 190 |
| Ref. Ex. 16 | BH1-16 | BD1 | 5 | BH2 | BD1 | 20 | 3.30 | 10.5 | 192 |
| Ref. Ex. 17 | BH1-17 | BD1 | 5 | BH2 | BD1 | 20 | 3.38 | 10.2 | 185 |
| Ref. Ex. 18 | BH1-18 | BD1 | 5 | BH2 | BD1 | 20 | 3.41 | 10.6 | 204 |
| Ref. Ex. 19 | BH1-19 | BD1 | 5 | BH2 | BD1 | 20 | 3.39 | 10.3 | 191 |
| Ref. Ex. 20 | R-BH1 | BD1 | 5 | BH2 | BD1 | 20 | 3.91 | 10.1 | — |

TABLE 3

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Comp. Ref. Ex. 1 | BH1 | BD1 | 25 | — | — | — | — | 7.6 | 65 |
| Comp. Ref. Ex. 2 | — | — | — | BH2 | BD1 | 25 | — | 9.9 | 167 |
| Comp. Ref. Ex. 3 | BH1-2 | BD1 | 25 | — | — | — | — | 7.2 | 59 |
| Comp. Ref. Ex. 4 | BH1-3 | BD1 | 25 | — | — | — | — | 7.4 | 71 |
| Comp. Ref. Ex. 5 | BH1-4 | BD1 | 25 | — | — | — | — | 7.8 | 70 |
| Comp. Ref. Ex. 6 | BH1-5 | BD1 | 25 | — | — | — | — | 7.5 | 62 |
| Comp. Ref. Ex. 7 | BH1-6 | BD1 | 25 | — | — | — | — | 7.4 | 60 |
| Comp. Ref. Ex. 8 | BH1-7 | BD1 | 25 | — | — | — | — | 7.3 | 53 |
| Comp. Ref. Ex. 9 | BH1-8 | BD1 | 25 | — | — | — | — | 7.4 | 55 |
| Comp. Ref. Ex. 10 | BH1-9 | BD1 | 25 | — | — | — | — | 7.5 | 67 |
| Comp. Ref. Ex. 11 | BH1-10 | BD1 | 25 | — | — | — | — | 7.1 | 51 |
| Comp. Ref. Ex. 12 | BH1-11 | BD1 | 25 | — | — | — | — | 7.8 | 81 |
| Comp. Ref. Ex. 13 | BH1-12 | BD1 | 25 | — | — | — | — | 7.0 | 48 |
| Comp. Ref. Ex. 14 | BH1-13 | BD1 | 25 | — | — | — | — | 7.1 | 53 |
| Comp. Ref. Ex. 15 | BH1-14 | BD1 | 25 | — | — | — | — | 6.9 | 56 |
| Comp. Ref. Ex. 16 | BH1-15 | BD1 | 25 | — | — | — | — | 7.1 | 59 |
| Comp. Ref. Ex. 17 | BH1-16 | BD1 | 25 | — | — | — | — | 7.0 | 62 |
| Comp. Ref. Ex. 18 | BH1-17 | BD1 | 25 | — | — | — | — | 6.7 | 53 |
| Comp. Ref. Ex. 19 | BH1-18 | BD1 | 25 | — | — | — | — | 7.1 | 62 |
| Comp. Ref. Ex. 20 | BH1-19 | BD1 | 25 | — | — | — | — | 6.9 | 43 |
| Comp. Ref. Ex. 21 | BH1-20 | BD1 | 25 | — | — | — | — | 6.5 | 21 |

### Reference Example 21

An organic EL device in Reference Example 21 was manufactured in the same manner as in Reference Example 1 except that the compound BH2 (second host material) in the second emitting layer was replaced by a compound shown in Table 4.

### Reference Examples 22 and 23

Organic EL devices in Reference Examples 22 and 23 were manufactured in the same manner as in Reference Example 1 except that the compound BH1 (first host material) in the first emitting layer and the compound BH2 (second host material) in the second emitting layer were replaced by compounds shown in Table 4.

### Comparative Reference Example 22

An organic EL device in Comparative Reference Example 22 was manufactured in the same manner as in Comparative Reference Example 2 except that the compound BH2 (second host material) in the second emitting layer was replaced by a compound shown in Table 4.

TABLE 4

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 21 | BH1 | BD1 | 5 | BH2-2 | BD1 | 20 | 3.96 | 9.8 | 192 |
| Ref. Ex 22 | R-BH1 | BD1 | 5 | BH2-2 | BD1 | 20 | 4.40 | 9.4 | — |
| Ref. Ex. 23 | R-BH2 | BD1 | 5 | BH2-2 | BD1 | 20 | 4.68 | 9.5 | — |
| Comp. Ref. Ex. 1 | BH1 | BD1 | 25 | — | — | — | — | 7.6 | 65 |
| Comp. Ref. Ex. 22 | — | — | — | BH2-2 | BD1 | 25 | — | 9.2 | 115 |

### Reference Example 24

An organic EL device in Reference Example 24 was manufactured in the same manner as in Reference Example 1 except that the compound BH2 (second host material) in the second emitting layer was replaced by a compound shown in Table 5.

### Reference Examples 25 and 26

Organic EL devices in Reference Examples 25 and 26 were manufactured in the same manner as in Reference Example 1 except that the compound BH1 (first host material) in the first emitting layer and the compound BH2 (second host material) in the second emitting layer were replaced by compounds shown in Table 5.

### Comparative Reference Example 23

An organic EL device in Comparative Reference Example 23 was manufactured in the same manner as in Comparative Reference Example 2 except that the compound BH2 (second host material) in the second emitting layer was replaced by a compound shown in Table 5.

TABLE 5

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 24 | BH1 | BD1 | 5 | BH2-3 | BD1 | 20 | 3.54 | 10.6 | 278 |
| Ref. Ex. 25 | R-BH1 | BD1 | 5 | BH2-3 | BD1 | 20 | 3.98 | 10.1 | — |
| Ref. Ex. 26 | R-BH2 | BD1 | 5 | BH2-3 | BD1 | 20 | 4.26 | 10.2 | — |
| Comp. Ref. Ex. 1 | BH1 | BD1 | 25 | — | — | — | — | 7.6 | 65 |
| Comp. Ref. Ex. 23 | — | — | — | BH2-3 | BD1 | 25 | — | 9.9 | 182 |

### Reference Example 27

An organic EL device in Reference Example 27 was manufactured in the same manner as in Reference Example 1 except that the compound BH2 (second host material) in the second emitting layer was replaced by a compound shown in Table 6.

### Reference Examples 28 and 29

Organic EL devices in Reference Examples 28 and 29 were manufactured in the same manner as in Reference Example 1 except that the compound BH1 (first host material) in the first emitting layer and the compound BH2 (second host material) in the second emitting layer were replaced by compounds shown in Table 6.

### Comparative Reference Example 24

An organic EL device in Comparative Reference Example 24 was manufactured in the same manner as in Comparative Reference Example 2 except that the compound BH2 (second host material) in the second emitting layer was replaced by a compound shown in Table 6.

TABLE 6

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 27 | BH1 | BD1 | 5 | BH2-4 | BD1 | 20 | 3.26 | 8.1 | 272 |
| Ref. Ex. 28 | R-BH1 | BD1 | 5 | BH2-4 | BD1 | 20 | 3.70 | 7.9 | — |
| Ref. Ex. 29 | R-BH2 | BD1 | 5 | BH2-4 | BD1 | 20 | 3.98 | 7.9 | — |
| Comp. Ref. Ex. 1 | BH1 | BD1 | 25 | — | — | — | — | 7.6 | 65 |
| Comp. Ref. Ex. 24 | — | — | — | BH2-4 | BD1 | 25 | — | 7.7 | 114 |

### Reference Example 30

An organic EL device in Reference Example 30 was manufactured in the same manner as in Reference Example 1 except that the compound BH2 (second host material) in the second emitting layer was replaced by a compound shown in Table 7.

### Reference Examples 31 and 32

Organic EL devices in Reference Examples 31 and 32 were manufactured in the same manner as in Reference Example 1 except that the compound BH1 (first host material) in the first emitting layer and the compound BH2 (second host material) in the second emitting layer were replaced by compounds shown in Table 7.

### Comparative Reference Example 25

An organic EL device in Comparative Reference Example 25 was manufactured in the same manner as in Comparative Reference Example 2 except that the compound BH2 (second host material) in the second emitting layer was replaced by a compound shown in Table 7.

TABLE 7

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 30 | BH1 | BD1 | 5 | BH2-5 | BD1 | 20 | 3.76 | 8.0 | 196 |
| Ref. Ex. 31 | R-BH1 | BD1 | 5 | BH2-5 | BD1 | 20 | 4.20 | 7.8 | — |
| Ref. Ex. 32 | R-BH2 | BD1 | 5 | BH2-5 | BD1 | 20 | 4.48 | 7.8 | — |
| Comp. Ref. Ex. 1 | BH1 | BD1 | 25 | — | — | — | — | 7.6 | 65 |
| Comp. Ref. Ex. 25 | — | — | — | BH2-5 | BD1 | 25 | — | 7.6 | 92 |

### Reference Example 33

An organic EL device in Reference Example 33 was manufactured in the same manner as in Reference Example 1 except that the compound BH2 (second host material) in the second emitting layer was replaced by a compound shown in Table 8.

### Reference Examples 34 and 35

Organic EL devices in Reference Examples 34 and 35 were manufactured in the same manner as in Reference Example 1 except that the compound BH1 (first host material) in the first emitting layer and the compound BH2 (second host material) in the second emitting layer were replaced by compounds shown in Table 8.

### Comparative Reference Example 26

An organic EL device in Comparative Reference Example 26 was manufactured in the same manner as in Comparative Reference Example 2 except that the compound BH2 (second host material) in the second emitting layer was replaced by a compound shown in Table 8.

TABLE 8

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | L195 |
| | Compound | Compound | [nm] | Compound | Compound | [nm] | [V] | [%] | [hr] |
| Ref. Ex. 33 | BH1 | BD1 | 5 | BH2-6 | BD1 | 20 | 3.14 | 10.5 | 198 |
| Ref. Ex. 34 | R-BH1 | BD1 | 5 | BH2-6 | BD1 | 20 | 3.58 | 8.2 | — |
| Ref. Ex. 35 | R-BH2 | BD1 | 5 | BH2-6 | BD1 | 20 | 3.86 | 8.2 | — |
| Comp. Ref. Ex. 1 | BH1 | BD1 | 25 | — | — | — | — | 7.6 | 65 |
| Comp. Ref. Ex. 26 | — | — | — | BH2-6 | BD1 | 25 | — | 8.0 | 71 |

### Reference Example 36

An organic EL device in Reference Example 36 was manufactured in the same manner as in Reference Example 1 except that the compound BH2 (second host material) in the second emitting layer was replaced by a compound shown in Table 9.

### Reference Examples 37 and 38

Organic EL devices in Reference Examples 37 and 38 were manufactured in the same manner as in Reference Example 1 except that the compound BH1 (first host material) in the first emitting layer and the compound BH2 (second host material) in the second emitting layer were replaced by compounds shown in Table 9.

### Comparative Reference Example 27

An organic EL device in Comparative Reference Example 27 was manufactured in the same manner as in Comparative Reference Example 2 except that the compound BH2 (second host material) in the second emitting layer was replaced by a compound shown in Table 9.

TABLE 9

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 36 | BH1 | BD1 | 5 | BH2-7 | BD1 | 20 | 3.21 | 10.7 | 217 |
| Ref. Ex. 37 | R-BH1 | BD1 | 5 | BH2-7 | BD1 | 20 | 3.65 | 8.0 | — |
| Ref. Ex. 38 | R-BH2 | BD1 | 5 | BH2-7 | BD1 | 20 | 3.93 | 8.0 | — |
| Comp. Ref. Ex. 1 | BH1 | BD1 | 25 | — | — | — | — | 7.6 | 65 |
| Comp. Ref. Ex. 27 | — | — | — | BH2-7 | BD1 | 25 | — | 7.8 | 106 |

### Reference Example 39

An organic EL device in Reference Example 39 was manufactured in the same manner as in Reference Example 1 except that the compound BH2 (second host material) in the second emitting layer was replaced by a compound shown in Table 10.

### Reference Examples 40 and 41

Organic EL devices in Reference Examples 40 and 41 were manufactured in the same manner as in Reference Example 1 except that the compound BH1 (first host material) in the first emitting layer and the compound BH2 (second host material) in the second emitting layer were replaced by compounds shown in Table 10.

### Comparative Reference Example 28

An organic EL device in Comparative Reference Example 28 was manufactured in the same manner as in Comparative Reference Example 2 except that the compound BH2 (second host material) in the second emitting layer was replaced by a compound shown in Table 10.

TABLE 10

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 39 | BH1 | BD1 | 5 | BH2-8 | BD1 | 20 | 3.39 | 9.2 | 192 |
| Ref. Ex. 40 | R-BH1 | BD1 | 5 | BH2-8 | BD1 | 20 | 3.83 | 8.0 | — |
| Ref. Ex. 41 | R-BH2 | BD1 | 5 | BH2-8 | BD1 | 20 | 4.11 | 8.0 | — |
| Comp. Ref. Ex. 1 | BH1 | BD1 | 25 | — | — | — | — | 7.6 | 65 |
| Comp. Ref. Ex. 28 | — | — | — | BH2-8 | BD1 | 25 | — | 7.8 | 74 |

### Reference Example 42

An organic EL device in Reference Example 42 was manufactured in the same manner as in Reference Example 1 except that the compound BH2 (second host material) in the second emitting layer was replaced by a compound shown in Table 11.

### Reference Examples 43 and 44

Organic EL devices in Reference Examples 43 and 44 were manufactured in the same manner as in Reference Example 1 except that the compound BH1 (first host material) in the first emitting layer and the compound BH2 (second host material) in the second emitting layer were replaced by compounds shown in Table 11.

### Comparative Reference Example 29

An organic EL device in Comparative Reference Example 29 was manufactured in the same manner as in Comparative Reference Example 2 except that the compound BH2 (second host material) in the second emitting layer was replaced by a compound shown in Table 11.

TABLE 11

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 42 | BH1 | BD1 | 5 | BH2-9 | BD1 | 20 | 3.56 | 10.5 | 300 |
| Ref. Ex. 43 | R-BH1 | BD1 | 5 | BH2-9 | BD1 | 20 | 4.00 | 10.0 | — |
| Ref. Ex. 44 | R-BH2 | BD1 | 5 | BH2-9 | BD1 | 20 | 4.28 | 10.1 | — |
| Comp. Ref. Ex 1 | BH1 | BD1 | 25 | — | — | — | — | 7.6 | 65 |
| Comp. Ref. Ex. 29 | — | — | — | BH2-9 | BD1 | 25 | — | 9.8 | 195 |

### Reference Example 45

An organic EL device in Reference Example 45 was manufactured in the same manner as in Reference Example 1 except that the compound BD1 in the first emitting layer and the compound BD1 in the second emitting layer were replaced by compounds shown in Table 12.

### Reference Examples 46 and 47

Organic EL devices in Reference Examples 46 and 47 were manufactured in the same manner as in Reference Example 1 except that the compound BH1 (first host material) and the compound BD1 in the first emitting layer and the compound BD1 in the second emitting layer were replaced by compounds shown in Table 12.

### Comparative Reference Example 30

An organic EL device in Comparative Reference Example 30 was manufactured in the same manner as in Comparative Reference Example 1 except that the compound BD1 in the first emitting layer was replaced by a compound shown in Table 12.

### Comparative Reference Example 31

An organic EL device in Comparative Reference Example 31 was manufactured in the same manner as in Comparative Reference Example 2 except that the compound BD1 in the second emitting layer was replaced by a compound shown in Table 12.

TABLE 12

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 45 | BH1 | BD2 | 5 | BH2 | BD2 | 20 | 3.57 | 9.7 | 203 |
| Ref. Ex. 46 | R-BH1 | BD2 | 5 | BH2 | BD2 | 20 | 4.01 | 9.3 | — |
| Ref. Ex. 47 | R-BH2 | BD2 | 5 | BH2 | BD2 | 20 | 4.29 | 9.4 | — |
| Comp. Ref. Ex. 30 | BH1 | BD2 | 25 | — | — | — | — | 7.0 | 51 |
| Comp. Ref. Ex. 31 | — | — | — | BH2 | BD2 | 25 | — | 9.1 | 120 |

### Reference Example 48

An organic EL device in Reference Example 48 was manufactured in the same manner as in Reference Example 1 except that the compound BD1 in the first emitting layer and the compound BD1 in the second emitting layer were replaced by compounds shown in Table 13.

### Reference Examples 49 and 50

Organic EL devices in Reference Examples 49 and 50 were manufactured in the same manner as in Reference Example 1 except that the compound BH1 (first host material) and the compound BD1 in the first emitting layer and the compound BD1 in the second emitting layer were replaced by compounds shown in Table 13.

### Comparative Reference Example 32

An organic EL device in Comparative Reference Example 32 was manufactured in the same manner as in Comparative Reference Example 1 except that the compound BD1 in the first emitting layer was replaced by a compound shown in Table 13.

### Comparative Reference Example 33

An organic EL device in Comparative Reference Example 33 was manufactured in the same manner as in Comparative Reference Example 2 except that the compound BD1 in the second emitting layer was replaced by a compound shown in Table 13.

TABLE 13

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ref. Ex. 48 | BH1 | BD3 | 5 | BH2 | BD3 | 20 | 3.51 | 10.2 | 167 |
| Ref. Ex. 49 | R-BH1 | BD3 | 5 | BH2 | BD3 | 20 | 3.95 | 9.7 | — |
| Ref. Ex. 50 | R-BH2 | BD3 | 5 | BH2 | BD3 | 20 | 4.23 | 9.8 | — |
| Comp. Ref. Ex. 32 | BH1 | BD3 | 25 | — | — | — | — | 7.4 | 46 |
| Comp. Ref. Ex. 33 | — | — | — | BH2 | BD3 | 25 | — | 9.5 | 103 |

### Examples 51 to 68

Organic EL devices in Examples 51 to 68 were manufactured in the same manner as in Reference Example 1 except that the compound BH1 (first host material) in the first emitting layer was replaced by compounds shown in Table 14.

### Comparative Example C69

An organic EL device in Comparative Example C69 was manufactured in the same manner as in Reference Example 1 except that the compound BH1 (first host material) in the first emitting layer was replaced by a compound shown in Table 14.

TABLE 14

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ex. 51 | BH1-23 | BD1 | 5 | BH2 | BD1 | 20 | 10.2 | 198 |
| Ex. 52 | BH1-26 | BD1 | 5 | BH2 | BD1 | 20 | 10.3 | 214 |
| Ex. 53 | BH1-27 | BD1 | 5 | BH2 | BD1 | 20 | 10.6 | 239 |
| Ex. 54 | BH1-28 | BD1 | 5 | BH2 | BD1 | 20 | 10.5 | 222 |
| Ex. 55 | BH1-32 | BD1 | 5 | BH2 | BD1 | 20 | 10.4 | 207 |
| Ex. 56 | BH1-33 | BD1 | 5 | BH2 | BD1 | 20 | 10.3 | 205 |
| Ex. 57 | BH1-34 | BD1 | 5 | BH2 | BD1 | 20 | 10.5 | 213 |
| Ex. 58 | BH1-35 | BD1 | 5 | BH2 | BD1 | 20 | 10.4 | 198 |
| Ex. 59 | BH1-40 | BD1 | 5 | BH2 | BD1 | 20 | 10.4 | 221 |
| Ex. 60 | BH1-41 | BD1 | 5 | BH2 | BD1 | 20 | 10.7 | 248 |
| Ex. 61 | BH1-42 | BD1 | 5 | BH2 | BD1 | 20 | 10.5 | 232 |
| Ex. 62 | BH1-43 | BD1 | 5 | BH2 | BD1 | 20 | 10.6 | 211 |
| Ex. 63 | BH1-44 | BD1 | 5 | BH2 | BD1 | 20 | 10.5 | 205 |
| Ex. 64 | BH1-45 | BD1 | 5 | BH2 | BD1 | 20 | 10.4 | 230 |
| Ex. 65 | BH1-46 | BD1 | 5 | BH2 | BD1 | 20 | 10.8 | 249 |
| Ex. 66 | BH1-47 | BD1 | 5 | BH2 | BD1 | 20 | 10.6 | 217 |
| Ex. 67 | BH1-48 | BD1 | 5 | BH2 | BD1 | 20 | 10.6 | 243 |
| Ex. 68 | BH1-49 | BD1 | 5 | BH2 | BD1 | 20 | 10.7 | 268 |
| Ex. C69 | R-BH3 | BD1 | 5 | BH2 | BD1 | 20 | 10.1 | 183 |

### Comparative Examples 34 to 51

Organic EL devices in Comparative Examples 34 to 51 were manufactured in the same manner as in Comparative Reference Example 1 except that the compound BH1 (first host material) in the first emitting layer was replaced by a compound shown in Table 15.

TABLE 15

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Comp. Ex. 34 | BH1-23 | BD1 | 25 | — | — | — | 6.3 | 50 |
| Comp. Ex. 35 | BH1-26 | BD1 | 25 | — | — | — | 6.6 | 78 |
| Comp. Ex. 36 | BH1-27 | BD1 | 25 | — | — | — | 6.7 | 81 |
| Comp. Ex. 37 | BH1-28 | BD1 | 25 | — | — | — | 6.5 | 72 |
| Comp. Ex. 38 | BH1-32 | BD1 | 25 | — | — | — | 6.1 | 49 |
| Comp. Ex. 39 | BH1-33 | BD1 | 25 | — | — | — | 6.2 | 55 |
| Comp. Ex. 40 | BH1-34 | BD1 | 25 | — | — | — | 6.2 | 57 |
| Comp. Ex. 41 | BH1-35 | BD1 | 25 | — | — | — | 6.0 | 49 |
| Comp. Ex. 42 | BH1-40 | BD1 | 25 | — | — | — | 6.2 | 68 |
| Comp. Ex. 43 | BH1-41 | BD1 | 25 | — | — | — | 6.6 | 91 |
| Comp. Ex. 44 | BH1-42 | BD1 | 25 | — | — | — | 6.4 | 85 |
| Comp. Ex. 45 | BH1-43 | BD1 | 25 | — | — | — | 6.4 | 72 |
| Comp. Ex. 46 | BH1-44 | BD1 | 25 | — | — | — | 6.4 | 77 |
| Comp. Ex. 47 | BH1-45 | BD1 | 25 | — | — | — | 6.2 | 81 |
| Comp. Ex. 48 | BH1-46 | BD1 | 25 | — | — | — | 6.3 | 94 |
| Comp. Ex. 49 | BH1-47 | BD1 | 25 | — | — | — | 6.2 | 67 |
| Comp. Ex. 50 | BH1-48 | BD1 | 25 | — | — | — | 6.1 | 64 |
| Comp. Ex. 51 | BH1-49 | BD1 | 25 | — | — | — | 6.8 | 97 |
| Comp. Ref. Ex. 2 | — | — | — | BH2 | BD1 | 25 | 9.9 | 167 |

### Manufacture 2 of Organic EL Device

Organic EL devices were manufactured and evaluated as follows.

### Example 70

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

A compound BH1-21 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET4 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of an organic EL device in Example 70 is roughly shown as follows.

### ITO(130)/HA1(5)/HT3(80)/HT4(10)/BH1-21:BD1(5.98%:2%)/BH2:BD1(20.98%:2%)/ET4(10)/ET2(15)/LiF(1)/Al(80)

The numerals in parentheses represent film thickness (unit: nm).

The numerals (98%:2%) represented by percentage in the same parentheses indicate a ratio (mass %) between the host material (compound BH1-21 or BH2) and the compound BD1 in the first emitting layer or the second emitting layer. Similar notations apply to the description below.

### Examples 71 to 77

Organic EL devices in Examples 71 to 77 were manufactured in the same manner as in Example 70 except that the compound BH1-21 (first host material) in the first emitting layer was replaced by the first compound shown in Table 16.

### Comparative Example C78

An organic EL device in Comparative Example C78 was manufactured in the same manner as in Example 70 except that the compound BH1-21 (first host material) in the first emitting layer was replaced by the first compound shown in Table 16.

### Comparative Examples 52 to 59

Organic EL devices of Comparative Examples 52 to 59 were manufactured in the same manner as in Example 70 except that a 25-nm-thick first emitting layer was formed as the emitting layer, the first electron transporting layer was formed on the first emitting layer without forming the second emitting layer, and the first compound (first host material) in the first emitting layer was replaced by the first compound shown in Table 16.

### Comparative Example 60

An organic EL device of Comparative Example 60 was manufactured in the same manner as in Example 70 except that a 25-nm-thick second emitting layer was formed on the second hole transporting layer without forming the first emitting layer as shown in Table 16.

TABLE 16

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT95 |
| | Compound | Compound | [nm] | Compound | Compound | [nm] | [V] | [%] | [hr] |
| Ex. 70 | BH1-21 | BD1 | 5 | BH2 | BD1 | 20 | 3.40 | 8.7 | 160 |
| Ex. 71 | BH1-22 | BD1 | 5 | BH2 | BD1 | 20 | 3.46 | 9.0 | 225 |
| Ex. 72 | BH1-24 | BD1 | 5 | BH2 | BD1 | 20 | 3.27 | 8.4 | 79 |
| Ex. 73 | BH1-25 | BD1 | 5 | BH2 | BD1 | 20 | 3.35 | 8.7 | 174 |
| Ex. 74 | BH1-36 | BD1 | 5 | BH2 | BD1 | 20 | 3.39 | 8.5 | 125 |
| Ex. 75 | BH1-37 | BD1 | 5 | BH2 | BD1 | 20 | 3.44 | 8.8 | 135 |
| Ex. 76 | BH1-50 | BD1 | 5 | BH2 | BD1 | 20 | 3.42 | 8.5 | 111 |
| Ex. 77 | BH1-51 | BD1 | 5 | BH2 | BD1 | 20 | 3.31 | 8.4 | 105 |
| Comp. Ex. C78 | R-BH3 | BD1 | 5 | BH2 | BD1 | 20 | 3.53 | 7.9 | 36 |
| Comp. Ex. 52 | BH1-21 | BD1 | 25 | — | — | — | — | 6.2 | 32 |
| Comp. Ex. 53 | BH1-22 | BD1 | 25 | — | — | — | — | 6.4 | 45 |
| Comp. Ex. 54 | BH1-24 | BD1 | 25 | — | — | — | — | 6.0 | 13 |
| Comp. Ex. 55 | BH1-25 | BD1 | 25 | — | — | — | — | 6.2 | 25 |
| Comp. Ex. 56 | BH1-36 | BD1 | 25 | — | — | — | — | 6.1 | 25 |
| Comp. Ex. 57 | BH1-37 | BD1 | 25 | — | — | — | — | 6.3 | 27 |
| Comp. Ex. 58 | BH1-50 | BD1 | 25 | — | — | — | — | 6.1 | 21 |
| Comp. Ex. 59 | BH1-51 | BD1 | 25 | — | — | — | — | 6.0 | 19 |
| Comp. Ex. 60 | — | — | — | BH2 | BD1 | 25 | — | 7.7 | 56 |

### Manufacture 3 of Organic EL Device

Organic EL devices were manufactured and evaluated as follows.

### Example 79

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

A compound BH1-29 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

A compound ET3 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of an organic EL device in Example 79 is roughly shown as follows.

### ITO(130)/HA1(5)/HT3(80)/HT4(10)/BH1-29:BD1(5.98%:2%)/BH2:BD1(20.98%:2%)/ET3(10)/ET2(15)/LiF(1)/Al(80)

The numerals in parentheses represent film thickness (unit: nm).

The numerals (98%:2%) represented by percentage in the same parentheses indicate a ratio (mass %) between the host material (compound BH1-29 or BH2) and the compound BD1 in the first emitting layer or the second emitting layer. Similar notations apply to the description below.

### Examples 80 to 89

Organic EL devices in Examples 80 to 89 were manufactured in the same manner as in Example 79 except that the compound BH1-29 (first host material) in the first emitting layer was replaced by the first compound shown in Table 17.

### Comparative Example C90

An organic EL device in Comparative Example C90 was manufactured in the same manner as in Example 79 except that the compound BH1-29 (first host material) in the first emitting layer was replaced by the first compound shown in Table 17.

### Comparative Examples 61 to 71

Organic EL devices of Comparative Examples 61 to 71 were manufactured in the same manner as in Example 79 except that a 25-nm-thick first emitting layer was formed as the emitting layer, the first electron transporting layer was formed on the first emitting layer without forming the second emitting layer, and the first compound (first host material) in the first emitting layer was replaced by the first compound shown in Table 17.

### Comparative Example 72

An organic EL device of Comparative Example 72 was manufactured in the same manner as in Example 79 except that a 25-nm-thick second emitting layer was formed on the second hole transporting layer without forming the first emitting layer as shown in Table 17.

TABLE 17

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ex. 79 | BH1-29 | BD1 | 5 | BH2 | BD1 | 20 | 9.3 | 125 |
| Ex. 80 | BH1-30 | BD1 | 5 | BH2 | BD1 | 20 | 9.3 | 103 |
| Ex. 81 | BH1-31 | BD1 | 5 | BH2 | BD1 | 20 | 9.6 | 119 |
| Ex. 82 | BH1-38 | BD1 | 5 | BH2 | BD1 | 20 | 9.8 | 138 |
| Ex. 83 | BH1-39 | BD1 | 5 | BH2 | BD1 | 20 | 9.7 | 122 |
| Ex. 84 | BH1-52 | BD1 | 5 | BH2 | BD1 | 20 | 9.5 | 151 |
| Ex. 85 | BH1-53 | BD1 | 5 | BH2 | BD1 | 20 | 9.3 | 132 |
| Ex. 86 | BH1-54 | BD1 | 5 | BH2 | BD1 | 20 | 9.1 | 110 |
| Ex. 87 | BH1-55 | BD1 | 5 | BH2 | BD1 | 20 | 9.4 | 109 |
| Ex. 88 | BH1-56 | BD1 | 5 | BH2 | BD1 | 20 | 9.2 | 111 |
| Ex. 89 | BH1-57 | BD1 | 5 | BH2 | BD1 | 20 | 9.2 | 121 |
| Comp. Ex. C90 | R-BH3 | BD1 | 5 | BH2 | BD1 | 20 | 8.3 | 97 |
| Comp. Ex. 61 | BH1-29 | BD1 | 25 | — | — | — | 6.7 | 61 |
| Comp. Ex. 62 | BH1-30 | BD1 | 25 | — | — | — | 6.9 | 53 |
| Comp. Ex. 63 | BH1-31 | BD1 | 25 | — | — | — | 6.4 | 51 |
| Comp. Ex. 64 | BH1-38 | BD1 | 25 | — | — | — | 6.1 | 48 |
| Comp. Ex. 65 | BH1-39 | BD1 | 25 | — | — | — | 6.1 | 45 |
| Comp. Ex. 66 | BH1-52 | BD1 | 25 | — | — | — | 6.8 | 62 |
| Comp. Ex. 67 | BH1-53 | BD1 | 25 | — | — | — | 6.8 | 54 |
| Comp. Ex. 68 | BH1-54 | BD1 | 25 | — | — | — | 6.7 | 42 |
| Comp. Ex. 69 | BH1-55 | BD1 | 25 | — | — | — | 6.7 | 59 |
| Comp. Ex. 70 | BH1-56 | BD1 | 25 | — | — | — | 6.5 | 40 |
| Comp. Ex. 71 | BH1-57 | BD1 | 25 | — | — | — | 6.2 | 34 |
| Comp. Ex. 72 | — | — | — | BH2 | BD1 | 25 | 8.1 | 89 |

### Manufacture 4 of Organic EL Device

Organic EL devices were manufactured and evaluated as follows.

### Reference Example 91

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT5 and the compound HA2 were co-deposited on a surface provided with the transparent electrode line to cover the transparent electrode, thereby forming a 10-nm-thick hole injecting layer (HI). The ratios of the compound HT5 and the compound HA2 in the hole injecting layer were 97 mass % and 3 mass %, respectively.

After the formation of the hole injecting layer, the compound HT5 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-61 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET3 was vapor-deposited on the second emitting layer to form a 5-nm-thick first electron transporting layer (also referred to as a hole blocking layer (HBL)).

The compound ET6 and the compound Liq were co-deposited on the first electron transporting layer (HBL) to form a 25-nm-thick electron transporting layer (ET). The ratios of the compound ET6 and the compound Liq in the electron transporting layer (ET) were 50 mass % and 50 mass %, respectively. Liq is an abbreviation of (8-quinolinolato)lithium ((8-Quinolinolato)lithium).

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 91 is roughly shown as follows.

### ITO(130)/HT5:HA2(10.97%:3%)/HT5(85)/HT4(5)/BH1-61:BD1(5.98%:2%)/BH2:BD1(20.98%:2%)/ET3(5)/ET6:Liq(25.50%:50%)/Liq(1)/Al(80)

The numerals in parentheses represent film thickness (unit: nm).

The numerals (97%:3%) represented by percentage in the same parentheses indicate a ratio (mass %) between the compound HT5 and the compound HA2 in the hole injecting layer, the numerals (98%:2%) represented by percentage in the same parentheses indicate a ratio (mass %) between the host material (compound BH1-61 or BH2) and the dopant material (compound BD1) in the first emitting layer or the second emitting layer, and the numerals (50%:50%) represented by percentage in the same parentheses indicate a ratio (mass %) between the compound ET6 and the compound Liq in the electron transporting layer (ET). Similar notations apply to the description below.

### Reference Examples 92 to 95

Organic EL devices in Reference Examples 92 to 95 were manufactured in the same manner as in Reference Example 91 except that the compound BH1-61 (first host material) in the first emitting layer was replaced by the first compound shown in Table 18.

### Comparative Reference Examples 73 to 76

Organic EL devices in Comparative Reference Examples 73 to 76 were manufactured in the same manner as in Reference Example 91 except that a 25-nm-thick first emitting layer was formed as the emitting layer, the first electron transporting layer was formed on the first emitting layer without forming the second emitting layer, and the first compound (first host material) in the first emitting layer was replaced by the first compound shown in Table 18.

### Comparative Reference Example 77

An organic EL device in Comparative Reference Example 77 was manufactured in the same manner as in Reference Example 91 except that a 25-nm-thick second emitting layer was formed on the second hole transporting layer without forming the first emitting layer as shown in Table 18.

TABLE 18

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ref. Ex. 91 | BH1-61 | BD1 | 5 | BH2 | BD1 | 20 | 9.2 | 128 |
| Ref. Ex. 92 | BH1-62 | BD1 | 5 | BH2 | BD1 | 20 | 9.7 | 153 |
| Ref. Ex. 93 | BH1-63 | BD1 | 5 | BH2 | BD1 | 20 | 9.5 | 144 |
| Ref. Ex. 94 | BH1-69 | BD1 | 5 | BH2 | BD1 | 20 | 9.0 | 110 |
| Ref. Ex. 95 | R-BH3 | BD1 | 5 | BH2 | BD1 | 20 | 8.8 | 101 |
| Comp. Ref. Ex. 73 | BH1-61 | BD1 | 25 | — | — | — | 6.1 | 47 |
| Comp. Ref. Ex. 74 | BH1-62 | BD1 | 25 | — | — | — | 6.4 | 64 |
| Comp. Ref. Ex. 75 | BH1-63 | BD1 | 25 | — | — | — | 6.3 | 60 |
| Comp. Ref. Ex. 76 | BH1-69 | BD1 | 25 | — | — | — | 5.9 | 19 |
| Comp. Ref. Ex. 77 | — | — | — | BH2 | BD1 | 25 | 8.4 | 72 |

### Manufacture 5 of Organic EL Device

Organic EL devices were manufactured and evaluated as follows.

### Reference Example 96

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT3 and the compound HA2 were co-deposited on a surface provided with the transparent electrode line to cover the transparent electrode, thereby forming a 10-nm-thick hole injecting layer (HI). The ratios of the compound HT3 and the compound HA2 in the hole injecting layer were 97 mass % and 3 mass %, respectively.

After the formation of the hole injecting layer, the compound HT3 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-75 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET3 was vapor-deposited on the second emitting layer to form a 5-nm-thick first electron transporting layer (also referred to as a hole blocking layer (HBL)).

The compound ET8 and the compound Liq were co-deposited on the first electron transporting layer (HBL) to form a 25-nm-thick electron transporting layer (ET). The ratios of the compound ET5 and the compound Liq in the electron transporting layer (ET) were 50 mass % and 50 mass %, respectively. Liq is an abbreviation of (8-quinolinolato)lithium ((8-Quinolinolato)lithium).

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 96 is roughly shown as follows.

### ITO(130)/HT3:HA2(10.97%:3%)/HT3(85)/HT4(5)/BH1-75:BD1(5.98%:2%)/BH2:BD1(20.98%:2%)/ET3(5)/ET8:Liq(25.50%:50%)/Liq(1)/Al(80)

The numerals in parentheses represent film thickness (unit: nm).

The numerals (97%:3%) represented by percentage in the same parentheses indicate a ratio (mass %) between the compound HT3 and the compound HA2 in the hole injecting layer, the numerals (98%:2%) represented by percentage in the same parentheses indicate a ratio (mass %) between the host material (compound BH1-75 or BH2) and the dopant material (compound BD1) in the first emitting layer or the second emitting layer, and the numerals (50%:50%) represented by percentage in the same parentheses indicate a ratio (mass %) between the compound ET8 and the compound Liq in the electron transporting layer (ET). Similar notations apply to the description below.

### Reference Example 97

An organic EL device in Reference Example 97 was manufactured in the same manner as in Reference Example 96 except that the compound BH1-75 (first host material) in the first emitting layer was replaced by the first compound shown in Table 19.

### Comparative Reference Example 78

An organic EL device in Comparative Reference Example 78 was manufactured in the same manner as in Reference Example 96 except that a 25-nm-thick first emitting layer was formed as the emitting layer, the first electron transporting layer was formed on the first emitting layer without forming the second emitting layer, and the first compound (first host material) in the first emitting layer was replaced by the first compound shown in Table 19.

### Comparative Reference Example 79

An organic EL device in Comparative Reference Example 79 was manufactured in the same manner as in Reference Example 96 except that a 25-nm-thick second emitting layer was formed on the second hole transporting layer without forming the first emitting layer as shown in Table 19.

TABLE 19

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ref. Ex. 96 | BH1-75 | BD1 | 5 | BH2 | BD1 | 20 | 9.2 | 169 |
| Ref. Ex. 97 | R-BH3 | BD1 | 5 | BH2 | BD1 | 20 | — | 118 |
| Comp. Ref. Ex. 78 | BH1-75 | BD1 | 25 | — | — | — | 6.0 | 63 |
| Comp. Ref. Ex. 79 | — | — | — | BH2 | BD1 | 25 | 8.1 | 91 |

### Manufacture 6 of Organic EL Device

Organic EL devices were manufactured and evaluated as follows.

### Reference Example 98

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT5 and the compound HA2 were co-deposited on a surface provided with the transparent electrode line to cover the transparent electrode, thereby forming a 10-nm-thick hole injecting layer (HI). The ratios of the compound HT5 and the compound HA2 in the hole injecting layer were 97 mass % and 3 mass %, respectively.

After the formation of the hole injecting layer, the compound HT5 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-64 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET3 was vapor-deposited on the second emitting layer to form a 5-nm-thick first electron transporting layer (also referred to as a hole blocking layer (HBL)).

The compound ET8 and the compound Liq were co-deposited on the first electron transporting layer (HBL) to form a 25-nm-thick electron transporting layer (ET). The ratios of the compound ET8 and the compound Liq in the electron transporting layer (ET) were 50 mass % and 50 mass %, respectively.

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 98 is roughly shown as follows.

### ITO(130)/HT5:HA2(10.97%:3%)/HT5(85)/HT4(5)/BH1-64:BD1(5.98%:2%)/BH2:BD1(20.98%:2%)/ET3(5)/ET8:Liq(25.50%:50%)/Liq(1)/Al(80)

The numerals in parentheses represent film thickness (unit: nm).

The numerals (97%:3%) represented by percentage in the same parentheses indicate a ratio (mass %) between the compound HT5 and the compound HA2 in the hole injecting layer, the numerals (98%:2%) represented by percentage in the same parentheses indicate a ratio (mass %) between the host material (compound BH1-64 or BH2) and the dopant material (compound BD1) in the first emitting layer or the second emitting layer, and the numerals (50%:50%) represented by percentage in the same parentheses indicate a ratio (mass %) between the compound ET8 and the compound Liq in the electron transporting layer (ET). Similar notations apply to the description below.

### Reference Examples 99 to 103

Organic EL devices in Reference Examples 99 to 103 were manufactured in the same manner as in Reference Example 98 except that the compound BH1-64 (first host material) in the first emitting layer was replaced by the first compound shown in Table 20.

### Comparative Reference Examples 80 to 84

Organic EL devices in Comparative Reference Examples 80 to 84 were manufactured in the same manner as in Reference Example 98 except that a 25-nm-thick first emitting layer was formed as the emitting layer, the first electron transporting layer was formed on the first emitting layer without forming the second emitting layer, and the first compound (first host material) in the first emitting layer was replaced by the first compound shown in Table 20.

### Comparative Reference Example 85

An organic EL device in Comparative Reference Example 85 was manufactured in the same manner as in Reference Example 98 except that a 25-nm-thick second emitting layer was formed on the second hole transporting layer without forming the first emitting layer as shown in Table 20.

TABLE 20

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ref. Ex. 98 | BH1-64 | BD1 | 5 | BH2 | BD1 | 20 | 9.6 | 106 |
| Ref. Ex. 99 | BH1-65 | BD1 | 5 | BH2 | BD1 | 20 | 9.7 | 112 |
| Ref. Ex. 100 | BH1-66 | BD1 | 5 | BH2 | BD1 | 20 | 9.5 | 83 |
| Ref. Ex. 101 | BH1-67 | BD1 | 5 | BH2 | BD1 | 20 | 9.4 | 93 |
| Ref. Ex. 102 | BH1-68 | BD1 | 5 | BH2 | BD1 | 20 | 9.5 | 101 |
| Ref. Ex. 103 | R-BH3 | BD1 | 5 | BH2 | BD1 | 20 | 9.1 | — |
| Comp. Ref. Ex. 80 | BH1-64 | BD1 | 25 | — | — | — | 6.1 | 31 |
| Comp. Ref. Ex. 81 | BH1-65 | BD1 | 25 | — | — | — | 6.3 | 48 |
| Comp. Ref. Ex. 82 | BH1-66 | BD1 | 25 | — | — | — | 6.1 | 31 |
| Comp. Ref. Ex. 83 | BH1-67 | BD1 | 25 | — | — | — | 6.3 | 55 |
| Comp. Ref. Ex. 84 | BH1-68 | BD1 | 25 | — | — | — | 6.0 | 28 |
| Comp. Ref. Ex. 85 | — | — | — | BH2 | BD1 | 25 | 8.6 | 61 |

### Manufacture 7 of Organic EL Device

Organic EL devices were manufactured and evaluated as follows.

### Example 104

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HT5 and the compound HA2 were co-deposited on a surface provided with the transparent electrode line to cover the transparent electrode, thereby forming a 10-nm-thick hole injecting layer (HI). The ratios of the compound HT5 and the compound HA2 in the hole injecting layer were 97 mass % and 3 mass %, respectively.

After the formation of the hole injecting layer, the compound HT5 was vapor-deposited to form an 85-nm-thick first hole transporting layer (HT).

After the formation of the first hole transporting layer, the compound HT4 was vapor-deposited to form a 5-nm-thick second hole transporting layer (also referred to as an electron blocking layer (EBL)).

A compound BH1-70 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 5-nm-thick first electron transporting layer (also referred to as a hole blocking layer (HBL)).

The compound ET6 and the compound Liq were co-deposited on the first electron transporting layer (HBL) to form a 25-nm-thick electron transporting layer (ET). The ratios of the compound ET6 and the compound Liq in the electron transporting layer (ET) were 50 mass % and 50 mass %, respectively.

Liq was vapor-deposited on the second electron transporting layer to form a 1-nm-thick electron injecting layer.

Metal aluminum (Al) was vapor-deposited on the electron injecting layer to form an 80-nm-thick cathode.

A device arrangement of an organic EL device in Example 104 is roughly shown as follows.

### ITO(130)/HT5:HA2(10.97%:3%)/HT5(85)/HT4(5)/BH1-70:BD1(5.98%:2%)/BH2:BD1(20.98%:2%)/ET1(5)/ET6:Liq(25.50%:50%)/Liq(1)/Al(80)

The numerals in parentheses represent film thickness (unit: nm).

The numerals (97%:3%) represented by percentage in the same parentheses indicate a ratio (mass %) between the compound HT5 and the compound HA2 in the hole injecting layer, the numerals (98%:2%) represented by percentage in the same parentheses indicate a ratio (mass %) between the host material (compound BH1-70 or BH2) and the dopant material (compound BD1) in the first emitting layer or the second emitting layer, and the numerals (50%:50%) represented by percentage in the same parentheses indicate a ratio (mass %) between the compound ET6 and the compound Liq in the electron transporting layer (ET). Similar notations apply to the description below.

### Examples 105 to 108

Organic EL devices in Examples 105 to 108 were manufactured in the same manner as in Example 104 except that the compound BH1-70 (first host material) in the first emitting layer was replaced by the first compound shown in Table 21.

### Comparative Example C109

An organic EL device in Comparative Example C109 was manufactured in the same manner as in Example 104 except that the compound BH1-70 (first host material) in the first emitting layer was replaced by the first compound shown in Table 21.

### Comparative Examples 86 to 90

Organic EL devices of Comparative Examples 86 to 90 were manufactured in the same manner as in Example 104 except that a 25-nm-thick first emitting layer was formed as the emitting layer, the first electron transporting layer was formed on the first emitting layer without forming the second emitting layer, and the first compound (first host material) in the first emitting layer was replaced by the first compound shown in Table 21.

### Comparative Example 91

An organic EL device of Comparative Example 91 was manufactured in the same manner as in Example 104 except that a 25-nm-thick second emitting layer was formed on the second hole transporting layer without forming the first emitting layer as shown in Table 21.

TABLE 21

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ex. 104 | BH1-70 | BD1 | 5 | BH2 | BD1 | 20 | 10.2 | 185 |
| Ex. 105 | BH1-71 | BD1 | 5 | BH2 | BD1 | 20 | 10.7 | 223 |
| Ex. 106 | BH1-72 | BD1 | 5 | BH2 | BD1 | 20 | 10.4 | 212 |
| Ex. 107 | BH1-73 | BD1 | 5 | BH2 | BD1 | 20 | 10.6 | 220 |
| Ex. 108 | BH1-74 | BD1 | 5 | BH2 | BD1 | 20 | 10.3 | 218 |
| Comp. Ex. C109 | R-BH3 | BD1 | 5 | BH2 | BD1 | 20 | 8.7 | 101 |
| Comp. Ex. 86 | BH1-70 | BD1 | 25 | — | — | — | 6.2 | 59 |
| Comp. Ex. 87 | BH1-71 | BD1 | 25 | — | — | — | 6.6 | 63 |
| Comp. Ex. 88 | BH1-72 | BD1 | 25 | — | — | — | 6.5 | 51 |
| Comp. Ex. 89 | BH1-73 | BD1 | 25 | — | — | — | 6.5 | 62 |
| Comp. Ex. 90 | BH1-74 | BD1 | 25 | — | — | — | 6.4 | 60 |
| Comp. Ex. 91 | — | — | — | BH2 | BD1 | 25 | 8.3 | 76 |

### Manufacture 8 of Organic EL Device

### Reference Example 110

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, a compound HT8 was vapor-deposited to form a 10-nm-thick second hole transporting layer (also referred to as an electron blocking layer (EBL)).

A compound BH1-81 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 110 is roughly shown as follows.

### ITO(130)/HA1(5)/HT1(80)/HT8(10)/BH1-81:BD1(5.98%:2%)/BH2:BD1(20.98%:2%)/ET1(10)/ET2(15)/LiF(1)/Al(80)

The numerals in parentheses represent film thickness (unit: nm).

The numerals (98%:2%) represented by percentage in the same parentheses indicate a ratio (mass %) between the host material (compound BH1-81 or BH2) and the compound BD1 in the first emitting layer or the second emitting layer. Similar notations apply to the description below.

### Reference Example 111

An organic EL device in Reference Example 111 was manufactured in the same manner as in Reference Example 110 except that the compound BH1-81 (first host material) in the first emitting layer was replaced by the first compound shown in Table 22.

### Comparative Reference Example 92

An organic EL device in Comparative Reference Example 92 was manufactured in the same manner as in Reference Example 110 except that a 25-nm-thick first emitting layer was formed as the emitting layer, and the first electron transporting layer was formed on the first emitting layer without forming the second emitting layer.

### Comparative Reference Example 93

An organic EL device in Comparative Reference Example 93 was manufactured in the same manner as in Reference Example 110 except that a 25-nm-thick second emitting layer was formed on the second hole transporting layer without forming the first emitting layer as shown in Table 22.

TABLE 22

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ref. Ex. 110 | BH1-81 | BD1 | 5 | BH2 | BD1 | 20 | 10.7 | 134 |
| Ref. Ex. 111 | R-BH3 | BD1 | 5 | BH2 | BD1 | 20 | 10.4 | — |
| Comp. Ref. Ex. 92 | BH1-81 | BD1 | 25 | — | — | — | 6.4 | 35 |
| Comp. Ref. Ex. 93 | — | — | — | BH2 | BD1 | 25 | 10.2 | 102 |

### Manufacture 9 of Organic EL Device

### Reference Examples 112 and 113

Organic EL devices in Reference Examples 112 and 113 were manufactured in the same manner as in Reference Example 1 except that the compound BH1 (first host material) in the first emitting layer was replaced by a compound shown in Table 23.

### Comparative Reference Example 94

An organic EL device in Comparative Reference Example 94 was manufactured in the same manner as in Comparative Reference Example 1 except that the compound BH1 (first host material) in the first emitting layer was replaced by a first compound shown in Table 23.

TABLE 23

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ref. Ex. 112 | BH1-82 | BD1 | 5 | BH2 | BD1 | 20 | 10.4 | 219 |
| Ref. Ex. 113 | R-BH3 | BD1 | 5 | BH2 | BD1 | 20 | 10.1 | 183 |
| Comp. Ref. Ex. 94 | BH1-82 | BD1 | 25 | — | — | — | 6.2 | 71 |
| Comp. Ref. Ex. 2 | — | — | — | BH2 | BD1 | 25 | 9.9 | 167 |

### Manufacture 10 of Organic EL Device

### Reference Example 114

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

A compound BH1-83 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 114 is roughly shown as follows.

### ITO(130)/HA1(5)/HT1(80)/HT2(10)/BH1-83:BD1(5.98%:2%)/BH2:BD1(20.98%:2%)/ET7(10)/ET2(15)/LiF(1)/Al(80)

The numerals in parentheses represent film thickness (unit: nm).

The numerals (98%:2%) represented by percentage in the same parentheses indicate a ratio (mass %) between the host material (compound BH1-83 or BH2) and the compound BD1 in the first emitting layer or the second emitting layer. Similar notations apply to the description below.

### Reference Example 115

An organic EL device in Reference Example 115 was manufactured in the same manner as in Reference Example 114 except that the compound BH1-83 (first host material) in the first emitting layer was replaced by the first compound shown in Table 24.

### Comparative Reference Example 95

An organic EL device in Comparative Reference Example 95 was manufactured in the same manner as in Reference Example 114 except that a 25-nm-thick first emitting layer was formed as the emitting layer, and the first electron transporting layer was formed on the first emitting layer without forming the second emitting layer.

### Comparative Reference Example 96

An organic EL device in Comparative Reference Example 96 was manufactured in the same manner as in Reference Example 114 except that a 25-nm-thick second emitting layer was formed on the second hole transporting layer without forming the first emitting layer, as shown in Table 24.

TABLE 24

| | First Emitting Layer | | | Second Emitting Layer | | | | |
|---|---|---|---|---|---|---|---|---|
| | | | Film | | | Film | | |
| | First | Third | Thickness | Second | Fourth | Thickness | EQE | LT95 |
| | Compound | Compound | [nm] | Compound | Compound | [nm] | [%] | [hr] |
| Ref. Ex. 114 | BH1-83 | BD1 | 5 | BH2 | BD1 | 20 | 9.7 | 247 |
| Ref. Ex. 115 | R-BH3 | BD1 | 5 | BH2 | BD1 | 20 | 8.5 | — |
| Comp. Ref. Ex. 95 | BH1-83 | BD1 | 25 | — | — | — | 6.0 | 76 |
| Comp. Ref. Ex. 96 | — | — | — | BH2 | BD1 | 25 | 9.1 | 183 |

### Manufacture 11 of Organic EL Device

### Reference Example 116

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

The compound BH1 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 116 is roughly shown as follows.

### ITO(130)/HA1(5)/HT1(80)/HT4(10)/BH1:BD1(5.98%:2%)/BH2-8:BD1(20.98%:2%)/ET1(10)/ET2(20)/LiF(1)/Al(80)

The numerals in parentheses represent film thickness (unit: nm).

The numerals (98%:2%) represented by percentage in the same parentheses indicate a ratio (mass %) between the host material (compound BH1) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Reference Example 117

An organic EL device in Reference Example 117 was manufactured in the same manner as in Reference Example 116 except that the compound BH2-8 (second host material) in the second emitting layer was replaced by the second compound shown in Table 25.

### Comparative Reference Example 97

An organic EL device in Comparative Reference Example 97 was manufactured in the same manner as in Reference Example 116 except that a 25-nm-thick second emitting layer was formed on the second hole transporting layer without forming the first emitting layer as shown in Table 25, and the second compound (second host material) in the second emitting layer was replaced by the second compound shown in Table 25.

TABLE 25

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Ref. Ex. 116 | BH1 | BD1 | 5 | BH2-8 | BD1 | 20 | 3.4 | 9.8 | 120 |
| Ref. Ex. 117 | BH1 | BD1 | 5 | BH2-5 | BD1 | 20 | 3.6 | 10.1 | 160 |
| Comp. Ref. Ex. 97 | — | — | — | BH2-5 | BD1 | 25 | 3.8 | 8.9 | 110 |

### Manufacture 12 of Organic EL Device

### Reference Examples 118 and 119

The organic EL devices in Reference Examples 118 and 119 were manufactured in the same manner as in Reference Example 116 except that the compound BH2-8 (second host material) in the second emitting layer was replaced by the second compound shown in Table 26.

### Comparative Reference Example 98

An organic EL device in Comparative Reference Example 98 was manufactured in the same manner as in Reference Example 116 except that a 25-nm-thick second emitting layer was formed on the second hole transporting layer without forming the first emitting layer as shown in Table 26, and the second compound (second host material) in the second emitting layer was replaced by the second compound shown in Table 26.

TABLE 26

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Ref. Ex. 118 | BH1 | BD1 | 5 | BH2-2 | BD1 | 20 | 3.8 | 10.5 | 200 |
| Ref. Ex. 119 | BH1 | BD1 | 5 | BH2-10 | BD1 | 20 | 3.8 | 10.5 | 240 |
| Comp. Ref. Ex. 98 | — | — | — | BH2-10 | BD1 | 25 | 4.0 | 9.8 | 140 |

### Reference Example 120

The organic EL device in Reference Example 120 was manufactured in the same manner as in Reference Example 116 except that the compound BH2-8 (second host material) in the second emitting layer was replaced by the second compound shown in Table 27.

### Comparative Reference Example 99

An organic EL device in Comparative Reference Example 99 was manufactured in the same manner as in Reference Example 116 except that a 25-nm-thick second emitting layer was formed on the second hole transporting layer without forming the first emitting layer as shown in Table 27, and the second compound (second host material) in the second emitting layer was replaced by the second compound shown in Table 27.

TABLE 27

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Ref. Ex. 116 | BH1 | BD1 | 5 | BH2-8 | BD1 | 20 | 3.4 | 9.8 | 120 |
| Ref. Ex. 120 | BH1 | BD1 | 5 | BH2-11 | BD1 | 20 | 3.4 | 9.8 | 150 |
| Comp. Ref. Ex. 99 | — | — | — | BH2-11 | BD1 | 25 | 3.6 | 7.5 | 100 |

### Manufacture 13 of Organic EL Device

### Reference Example 121

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT4 was vapor-deposited to form a 10-nm-thick second hole transporting layer (also referred to as an electron blocking layer (EBL)).

The compound BH1 (first host material (BH)) and a compound BD2 (dopant material (BD)) were co-deposited on the second hole transporting layer such that the ratio of the compound BD2 accounted for 2 mass %, thereby forming a 5-nm-thick first emitting layer.

The compound BH2-2 (second host material (BH)) and the compound BD2 (dopant material (BD)) were co-deposited on the first emitting layer such that the ratio of the compound BD2 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 121 is roughly shown as follows.

### ITO(130)/HA1(5)/HT3(80)/HT4(10)/BH1:BD2(5.98%:2%)/BH2-2:BD2(20.98%:2%)/ET7(10)/ET2(20)/LiF(1)/Al(80)

The numerals in parentheses represent film thickness (unit: nm).

The numerals (98%:2%) represented by percentage in the same parentheses indicate a ratio (mass %) between the host material (compound BH1) and the compound BD2 in the first emitting layer, and numerals (98%:2%) represented by percentage in the same parentheses indicate a ratio (mass %) between the host material (compound BH2-2) and the compound BD2 in the second emitting layer. Similar notations apply to the description below.

### Reference Example 122

The organic EL device in Reference Example 122 was manufactured in the same manner as in Reference Example 121 except that the compound BH2-2 (second host material) in the second emitting layer was replaced by the second compound shown in Table 28.

### Comparative Reference Example 100

An organic EL device in Comparative Reference Example 100 was manufactured in the same manner as in Reference Example 121 except that a 25-nm-thick second emitting layer was formed on the second hole transporting layer without forming the first emitting layer as shown in Table 28, and the second compound (second host material) in the second emitting layer was replaced by the second compound shown in Table 28.

TABLE 28

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Ref. Ex. 121 | BH1 | BD2 | 5 | BH2-2 | BD2 | 20 | 3.8 | 10.1 | 180 |
| Ref. Ex. 122 | BH1 | BD2 | 5 | BH2-12 | BD2 | 20 | 4.0 | 10.3 | 200 |
| Comp. Ref. Ex. 100 | — | — | — | BH2-12 | BD2 | 25 | 4.2 | 8.8 | 110 |

### Manufacture 14 of Organic EL Device

### Reference Example 123

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT5 was vapor-deposited to form an 80-nm-thick first hole transporting layer (HT).

After the formation of the first hole transporting layer, a compound HT6 was vapor-deposited to form a 10-nm-thick second hole transporting layer (also referred to as an electron blocking layer (EBL)).

A compound BH1-10 (first host material (BH)) and the compound BD2 (dopant material (BD)) were co-deposited on the second hole transporting layer such that the ratio of the compound BD2 accounted for 2 mass %, thereby forming a 5-nm-thick first emitting layer.

The compound BH2-2 (second host material (BH)) and the compound BD2 (dopant material (BD)) were co-deposited on the first emitting layer such that the ratio of the compound BD2 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 123 is roughly shown as follows.

### ITO(130)/HA1(5)/HT5(80)/HT6(10)/BH1-10:BD2(5.98%:2%)/BH2-2:BD2(20.98%:2%)/ET7(10)/ET2(20)/LiF(1)/Al(80)

The numerals in parentheses represent film thickness (unit: nm).

The numerals (98%:2%) represented by percentage in the same parentheses indicate a ratio (mass %) between the host material (compound BH1-10) and the compound BD2 in the first emitting layer, and numerals (98%:2%) represented by percentage in the same parentheses indicate a ratio (mass %) between the host material (compound BH2-2) and the compound BD2 in the second emitting layer. Similar notations apply to the description below.

### Reference Example 124

An organic EL device in Reference Example 124 was manufactured in the same manner as in Reference Example 123 except that the compound BH2-2 (second host material) in the second emitting layer was replaced by the second compound shown in Table 29.

### Comparative Reference Example 101

An organic EL device in Comparative Reference Example 101 was manufactured in the same manner as in Reference Example 123 except that a 25-nm-thick second emitting layer was formed on the second hole transporting layer without forming the first emitting layer as shown in Table 29, and the second compound (second host material) in the second emitting layer was replaced by the second compound shown in Table 29.

TABLE 29

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Ref. Ex. 123 | BH1-10 | BD2 | 5 | BH2-2 | BD2 | 20 | 3.9 | 10.0 | 210 |
| Ref. Ex. 124 | BH1-10 | BD2 | 5 | BH2-13 | BD2 | 20 | 3.8 | 10.3 | 190 |
| Comp. Ref. Ex. 101 | — | — | — | BH2-13 | BD2 | 25 | 4.1 | 9.2 | 110 |

### Manufacture 15 of Organic EL Device

### Reference Example 125

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT7 was vapor-deposited to form a 10-nm-thick second hole transporting layer (also referred to as an electron blocking layer (EBL)).

The compound BH1-10 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-2 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 125 is roughly shown as follows.

### ITO(130)/HA1(5)/HT3(80)/HT7(10)/BH1-10:BD1(5.98%:2%)/BH2-2:BD1(20.98%:2%)/ET7(10)/ET2(20)/LiF(1)/Al(80)

The numerals in parentheses represent film thickness (unit: nm).

The numerals (98%:2%) represented by percentage in the same parentheses indicate a ratio (mass %) between the host material (compound BH1-10) and the compound BD1 in the first emitting layer, and numerals (98%:2%) represented by percentage in the same parentheses indicate a ratio (mass %) between the host material (compound BH2-2) and the compound BD1 in the second emitting layer. Similar notations apply to the description below.

### Reference Example 126

The organic EL device in Reference Example 126 was manufactured in the same manner as in Reference Example 125 except that the compound BH2-2 (second host material) in the second emitting layer was replaced by the second compound shown in Table 30.

### Comparative Reference Example 102

An organic EL device in Comparative Reference Example 102 was manufactured in the same manner as in Reference Example 125 except that a 25-nm-thick second emitting layer was formed on the second hole transporting layer without forming the first emitting layer as shown in Table 30, and the second compound (second host material) in the second emitting layer was replaced by the second compound shown in Table 30.

TABLE 30

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Ref. Ex. 125 | BH1-10 | BD1 | 5 | BH2-2 | BD1 | 20 | 4.0 | 10.5 | 150 |
| Ref. Ex. 126 | BH1-10 | BD1 | 5 | BH2-14 | BD1 | 20 | 4.0 | 10.8 | 160 |
| Comp. Ref. Ex. 102 | — | — | — | BH2-14 | BD1 | 25 | 4.2 | 9.5 | 100 |

### Reference Example 127

The organic EL device in Reference Example 127 was manufactured in the same manner as in Reference Example 125 except that the compound BH2-2 (second host material) in the second emitting layer was replaced by the second compound shown in Table 31.

### Comparative Reference Example 103

An organic EL device in Comparative Reference Example 103 was manufactured in the same manner as in Reference Example 125 except that a 25-nm-thick second emitting layer was formed on the second hole transporting layer without forming the first emitting layer as shown in Table 31, and the second compound (second host material) in the second emitting layer was replaced by the second compound shown in Table 31.

TABLE 31

| | First Emitting Layer | | | Second Emitting Layer | | | | | |
|---|---|---|---|---|---|---|---|---|---|
| | | | Film | | | Film | | | |
| | First | Third | Thickness | Second | Fourth | Thickness | Voltage | EQE | LT90 |
| | Compound | Compound | [nm] | Compound | Compound | [nm] | [V] | [%] | [hr] |
| Ref. Ex. 125 | BH1-10 | BD1 | 5 | BH2-2 | BD1 | 20 | 4.0 | 10.5 | 150 |
| Ref. Ex. 127 | BH1-10 | BD1 | 5 | BH2-15 | BD1 | 20 | 3.9 | 10.3 | 180 |
| Comp. Ref. Ex. 103 | — | — | — | BH2-15 | BD1 | 25 | 4.0 | 9.2 | 80 |

### Reference Example 128

The organic EL device in Reference Example 128 was manufactured in the same manner as in Reference Example 125 except that the compound BH2-2 (second host material) in the second emitting layer was replaced by the second compound shown in Table 32.

### Comparative Reference Example 104

An organic EL device in Comparative Reference Example 104 was manufactured in the same manner as in Reference Example 125 except that a 25-nm-thick second emitting layer was formed on the second hole transporting layer without forming the first emitting layer as shown in Table 32, and the second compound (second host material) in the second emitting layer was replaced by the second compound shown in Table 32.

TABLE 32

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 125 | BH1-10 | BD1 | 5 | BH2-2 | BD1 | 20 | 4.0 | 10.5 | 150 |
| Ref. Ex. 128 | BH1-10 | BD1 | 5 | BH2-16 | BD1 | 20 | 3.8 | 10.5 | 170 |
| Comp. Ref. | — | — | — | BH2-16 | BD1 | 25 | 4.1 | 9.5 | 70 |
| Ex. 104 | | | | | | | | | |

### Reference Example 129

The organic EL device in Reference Example 129 was manufactured in the same manner as in Reference Example 125 except that the compound BH2-2 (second host material) in the second emitting layer was replaced by the second compound shown in Table 33.

### Comparative Reference Example 105

An organic EL device in Comparative Reference Example 105 was manufactured in the same manner as in Reference Example 125 except that a 25-nm-thick second emitting layer was formed on the second hole transporting layer without forming the first emitting layer as shown in Table 33, and the second compound (second host material) in the second emitting layer was replaced by the second compound shown in Table 33.

TABLE 33

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 125 | BH1-10 | BD1 | 5 | BH2-2 | BD1 | 20 | 4.0 | 10.5 | 150 |
| Ref. Ex. 129 | BH1-10 | BD1 | 5 | BH2-17 | BD1 | 20 | 3.7 | 10.6 | 170 |
| Comp. Ref. | — | — | — | BH2-17 | BD1 | 25 | 4.0 | 9.1 | 60 |
| Ex. 105 | | | | | | | | | |

### Manufacture 16 of Organic EL Device

### Reference Example 130

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT3 was vapor-deposited to form an 80-nm-thick first hole transporting layer (HT).

After the formation of the first hole transporting layer, the compound HT7 was vapor-deposited to form a 10-nm-thick second hole transporting layer (also referred to as an electron blocking layer (EBL)).

The compound BH1-10 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET1 was vapor-deposited on the second emitting layer to form a 10-nm-thick first electron transporting layer (also referred to as a hole blocking layer (HBL)).

The compound ET5 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 130 is roughly shown as follows.

### ITO(130)/HA1(5)/HT3(80)/HT7(10)/BH1-10:BD1(5.98%:2%)/BH2-8:BD1(20.98%:2%)/ET1(10)/ET5(20)/LiF(1)/Al(80)

The numerals in parentheses represent film thickness (unit: nm).

The numerals (98%:2%) represented by percentage in the same parentheses indicate a ratio (mass %) between the host material (compound BH1-10) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Reference Example 131

An organic EL device in Reference Example 131 was manufactured in the same manner as in Reference Example 130 except that the compound BH2-8 (second host material) in the second emitting layer was replaced by the second compound shown in Table 34.

### Comparative Reference Example 106

An organic EL device in Comparative Reference Example 106 was manufactured in the same manner as in Reference Example 130 except that a 25-nm-thick second emitting layer was formed on the second hole transporting layer without forming the first emitting layer as shown in Table 34, and the second compound (second host material) in the second emitting layer was replaced by the second compound shown in Table 34.

TABLE 34

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 130 | BH1-10 | BD1 | 5 | BH2-8 | BD1 | 20 | 3.4 | 9.5 | 140 |
| Ref. Ex. 131 | BH1-10 | BD1 | 5 | BH2-18 | BD1 | 20 | 3.4 | 10.0 | 150 |
| Comp. Ref. | — | — | — | BH2-18 | BD1 | 25 | 3.6 | 9.0 | 100 |
| Ex. 106 | | | | | | | | | |

### Reference Example 132

An organic EL device in Reference Example 132 was manufactured in the same manner as in Reference Example 130 except that the compound BH2-8 (second host material) in the second emitting layer was replaced by the second compound shown in Table 35.

### Comparative Reference Example 107

An organic EL device in Comparative Reference Example 107 was manufactured in the same manner as in Reference Example 130 except that a 25-nm-thick second emitting layer was formed on the second hole transporting layer without forming the first emitting layer as shown in Table 35, and the second compound (second host material) in the second emitting layer was replaced by the second compound shown in Table 35.

TABLE 35

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 130 | BH1-10 | BD1 | 5 | BH2-8 | BD1 | 20 | 3.4 | 9.5 | 140 |
| Ref. Ex. 132 | BH1-10 | BD1 | 5 | BH2-19 | BD1 | 20 | 3.5 | 10.3 | 140 |
| Comp. Ref. | — | — | — | BH2-19 | BD1 | 25 | 3.6 | 9.2 | 80 |
| Ex. 107 | | | | | | | | | |

### Reference Example 133

An organic EL device in Reference Example 133 was manufactured in the same manner as in Reference Example 130 except that the compound BH2-8 (second host material) in the second emitting layer was replaced by the second compound shown in Table 36.

### Comparative Reference Example 108

An organic EL device in Comparative Reference Example 108 was manufactured in the same manner as in Reference Example 130 except that a 25-nm-thick second emitting layer was formed on the second hole transporting layer without forming the first emitting layer as shown in Table 36, and the second compound (second host material) in the second emitting layer was replaced by the second compound shown in Table 36.

TABLE 36

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 130 | BH1-10 | BD1 | 5 | BH2-8 | BD1 | 20 | 3.4 | 9.5 | 140 |
| Ref. Ex. 133 | BH1-10 | BD1 | 5 | BH2-20 | BD1 | 20 | 3.4 | 9.9 | 160 |
| Comp. Ref. | — | — | — | BH2-20 | BD1 | 25 | 3.7 | 8.8 | 120 |
| Ex. 108 | | | | | | | | | |

### Manufacture 17 of Organic EL Device

### Reference Example 134

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

The compound BH1 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET4 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 134 is roughly shown as follows.

### ITO(130)/HA1(5)/HT1(80)/HT2(10)/BH1:BD1(5.98%:2%)/BH2-8:BD1(20.98%:2%)/ET4(10)/ET2(20)/LiF(1)/Al(80)

The numerals in parentheses represent film thickness (unit: nm).

The numerals (98%:2%) represented by percentage in the same parentheses indicate a ratio (mass %) between the host material (compound BH1) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Reference Example 135

The organic EL device in Reference Example 135 was manufactured in the same manner as in Reference Example 134 except that the compound BH2-8 (second host material) in the second emitting layer was replaced by the second compound shown in Table 37.

### Comparative Reference Example 109

An organic EL device in Comparative Reference Example 109 was manufactured in the same manner as in Reference Example 134 except that a 25-nm-thick second emitting layer was formed on the second hole transporting layer without forming the first emitting layer as shown in Table 37, and the second compound (second host material) in the second emitting layer was replaced by the second compound shown in Table 37.

TABLE 37

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 135 | BH1 | BD1 | 5 | BH2-21 | BD1 | 20 | 3.3 | 9.6 | 130 |
| Comp. Ref. | — | — | — | BH2-21 | BD1 | 25 | 3.5 | 8.5 | 83 |
| Ex. 139 | | | | | | | | | |

### Reference Example 136

The organic EL device in Reference Example 136 was manufactured in the same manner as in Reference Example 134 except that the compound BH2-8 (second host material) in the second emitting layer was replaced by the second compound shown in Table 38.

### Comparative Reference Example 110

An organic EL device in Comparative Reference Example 110 was manufactured in the same manner as in Reference Example 134 except that a 25-nm-thick second emitting layer was formed on the second hole transporting layer without forming the first emitting layer as shown in Table 38, and the second compound (second host material) in the second emitting layer was replaced by the second compound shown in Table 38.

TABLE 38

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 136 | BH1 | BD1 | 5 | BH2-22 | BD1 | 20 | 3.4 | 8.3 | 140 |
| Comp. Ref. | — | — | — | BH2-22 | BD1 | 25 | 3.5 | 7.3 | 80 |
| Ex. 110 | | | | | | | | | |

### Reference Example 137

The organic EL device in Reference Example 137 was manufactured in the same manner as in Reference Example 134 except that the compound BH2-8 (second host material) in the second emitting layer was replaced by the second compound shown in Table 39.

### Comparative Reference Example 111

An organic EL device in Comparative Reference Example 111 was manufactured in the same manner as in Reference Example 134 except that a 25-nm-thick second emitting layer was formed on the second hole transporting layer without forming the first emitting layer as shown in Table 39, and the second compound (second host material) in the second emitting layer was replaced by the second compound shown in Table 39.

TABLE 39

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 137 | BH1 | BD1 | 5 | BH2-23 | BD1 | 20 | 3.3 | 8.8 | 130 |
| Comp. Ref. | — | — | — | BH2-23 | BD1 | 25 | 3.4 | 8.0 | 80 |
| Ex. 111 | | | | | | | | | |

### Reference Example 138

The organic EL device in Reference Example 138 was manufactured in the same manner as in Reference Example 134 except that the compound BH2-8 (second host material) in the second emitting layer was replaced by the second compound shown in Table 40.

### Comparative Reference Example 112

An organic EL device in Comparative Reference Example 112 was manufactured in the same manner as in Reference Example 134 except that a 25-nm-thick second emitting layer was formed on the second hole transporting layer without forming the first emitting layer as shown in Table 40, and the second compound (second host material) in the second emitting layer was replaced by the second compound shown in Table 40.

TABLE 40

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 138 | BH1 | BD1 | 5 | BH2-24 | BD1 | 20 | 3.5 | 9.1 | 120 |
| Comp. Ref. | — | — | — | BH2-24 | BD1 | 25 | 3.7 | 7.8 | 90 |
| Ex. 112 | | | | | | | | | |

### Reference Example 139

The organic EL device in Reference Example 139 was manufactured in the same manner as in Reference Example 134 except that the compound BH2-8 (second host material) in the second emitting layer was replaced by the second compound shown in Table 41.

### Comparative Reference Example 113

An organic EL device in Comparative Reference Example 113 was manufactured in the same manner as in Reference Example 134 except that a 25-nm-thick second emitting layer was formed on the second hole transporting layer without forming the first emitting layer as shown in Table 41, and the second compound (second host material) in the second emitting layer was replaced by the second compound shown in Table 41.

TABLE 41

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 139 | BH1 | BD1 | 5 | BH2-25 | BD1 | 20 | 3.4 | 9.4 | 130 |
| Comp. Ref. | — | — | — | BH2-25 | BD1 | 25 | 3.4 | 7.1 | 70 |
| Ex. 113 | | | | | | | | | |

### Reference Example 140

The organic EL device in Reference Example 140 was manufactured in the same manner as in Reference Example 134 except that the compound BH2-8 (second host material) in the second emitting layer was replaced by the second compound shown in Table 42.

### Comparative Reference Example 114

An organic EL device in Comparative Reference Example 114 was manufactured in the same manner as in Reference Example 134 except that a 25-nm-thick second emitting layer was formed on the second hole transporting layer without forming the first emitting layer as shown in Table 42, and the second compound (second host material) in the second emitting layer was replaced by the second compound shown in Table 42.

TABLE 42

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 140 | BH1 | BD1 | 5 | BH2-26 | BD1 | 20 | 3.5 | 9.2 | 130 |
| Comp. Ref. | — | — | — | BH2-26 | BD1 | 25 | 3.4 | 7.5 | 75 |
| Ex. 114 | | | | | | | | | |

### Reference Example 141

The organic EL device in Reference Example 141 was manufactured in the same manner as in Reference Example 134 except that the compound BH2-8 (second host material) in the second emitting layer was replaced by the second compound shown in Table 43.

### Comparative Reference Example 115

An organic EL device in Comparative Reference Example 115 was manufactured in the same manner as in Reference Example 134 except that a 25-nm-thick second emitting layer was formed on the second hole transporting layer without forming the first emitting layer as shown in Table 43, and the second compound (second host material) in the second emitting layer was replaced by the second compound shown in Table 43.

TABLE 43

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 141 | BH1 | BD1 | 5 | BH2-27 | BD1 | 20 | 3.2 | 9.1 | 130 |
| Comp. Ref. | — | — | — | BH2-27 | BD1 | 25 | 3.5 | 7.2 | 80 |
| Ex. 115 | | | | | | | | | |

### Reference Example 142

The organic EL device in Reference Example 142 was manufactured in the same manner as in Reference Example 134 except that the compound BH2-8 (second host material) in the second emitting layer was replaced by the second compound shown in Table 44.

### Comparative Reference Example 116

An organic EL device in Comparative Reference Example 116 was manufactured in the same manner as in Reference Example 134 except that a 25-nm-thick second emitting layer was formed on the second hole transporting layer without forming the first emitting layer as shown in Table 44, and the second compound (second host material) in the second emitting layer was replaced by the second compound shown in Table 44.

TABLE 44

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 134 | BH1 | BD1 | 5 | BH2-8 | BD1 | 20 | 3.3 | 9.8 | 90 |
| Ref. Ex. 142 | BH1 | BD1 | 5 | BH2-28 | BD1 | 20 | 3.3 | 9.0 | 140 |
| Comp. Ref. | — | — | — | BH2-28 | BD1 | 25 | 3.4 | 7.4 | 65 |
| Ex. 116 | | | | | | | | | |

### Manufacture 18 of Organic EL Device

### Reference Example 143

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, the compound HA1 was vapor-deposited on a surface provided with the transparent electrode line to cover the transparent electrode, thereby forming a 5-nm-thick hole injecting layer (HI).

After the formation of the hole injecting layer, the compound HT1 was vapor-deposited to form an 80-nm-thick first hole transporting layer (HT).

After the formation of the first hole transporting layer, the compound HT2 was vapor-deposited to form a 10-nm-thick second hole transporting layer (also referred to as an electron blocking layer (EBL)).

The compound BH1 (first host material (BH)) and the compound BD1 (dopant material (BD)) were co-deposited on the second hole transporting layer such that the ratio of the compound BD1 accounted for 2 mass %, thereby forming a 5-nm-thick first emitting layer.

The compound BH2-8 (second host material (BH)) and the compound BD1 (dopant material (BD)) were co-deposited on the first emitting layer such that the ratio of the compound BD1 accounted for 2 mass %, thereby forming a 20-nm-thick second emitting layer.

The compound ET7 was vapor-deposited on the second emitting layer to form a 10-nm-thick first electron transporting layer (also referred to as a hole blocking layer (HBL)).

The compound ET2 was vapor-deposited on the first electron transporting layer to form a 20-nm-thick second electron transporting layer (ET).

LiF was vapor-deposited on the second electron transporting layer to form a 1-nm-thick electron injecting layer.

Metal aluminum (Al) was vapor-deposited on the electron injecting layer to form an 80-nm-thick cathode.

A device arrangement of the organic EL device in Reference Example 143 is roughly shown as follows.

### ITO(130)/HA1(5)/HT1(80)/HT2(10)/BH1:BD1(5.98%:2%)/BH2-8:BD1(20.98%:2%)/ET7(10)/ET2(20)/LiF(1)/Al(80)

The numerals in parentheses represent film thickness (unit: nm).

The numerals (98%:2%) represented by percentage in the same parentheses indicate a ratio (mass %) between the host material (compound BH1) and the compound BD1 in the first emitting layer, and numerals (98%:2%) represented by percentage in the same parentheses indicate a ratio (mass %) between the host material (compound BH2-8) and the compound BD1 in the second emitting layer. Similar notations apply to the description below.

### Reference Example 144

An organic EL device in Reference Example 144 was manufactured in the same manner as in Reference Example 143 except that the compound BH2-8 (second host material) in the second emitting layer was replaced by the second compound shown in Table 45.

### Comparative Reference Example 117

An organic EL device in Comparative Reference Example 117 was manufactured in the same manner as in Reference Example 143 except that a 25-nm-thick second emitting layer was formed on the second hole transporting layer without forming the first emitting layer as shown in Table 45, and the second compound (second host material) in the second emitting layer was replaced by the second compound shown in Table 45.

TABLE 45

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | Voltage | EQE | LT90 |
| | pound | pound | [nm] | pound | pound | [nm] | [V] | [%] | [hr] |
| Ref. Ex. 143 | BH1 | BD1 | 5 | BH2-8 | BD1 | 20 | 3.5 | 9.0 | 120 |
| Ref. Ex. 144 | BH1 | BD1 | 5 | BH2-29 | BD1 | 20 | 4.0 | 10.1 | 80 |
| Comp. Ref. | — | — | — | BH2-29 | BD1 | 25 | 4.5 | 8.2 | 40 |
| Ex. 117 | | | | | | | | | |

### Manufacture 19 of Organic EL Device

### Example 145

A glass substrate (size: 25 mm×75 mm×1.1 mm thick, manufactured by Geomatec Co., Ltd.) having an ITO (Indium Tin Oxide) transparent electrode (anode) was ultrasonic-cleaned in isopropyl alcohol for five minutes, and then UV-ozone-cleaned for 30 minutes. The film thickness of the ITO transparent electrode was 130 nm.

The cleaned glass substrate having the transparent electrode line was attached to a substrate holder of a vacuum deposition apparatus. Initially, a compound HA3 was vapor-deposited on a surface provided with the transparent electrode line to cover the transparent electrode, thereby forming a 10-nm-thick hole injecting layer (HI).

After the formation of the hole injecting layer, the compound HT9 was vapor-deposited to form an 80-nm-thick first hole transporting layer (HT).

After the formation of the first hole transporting layer, a compound HT10 was vapor-deposited to form a 10-nm-thick second hole transporting layer (also referred to as an electron blocking layer (EBL)).

A compound BH1-84 (first host material (BH)) and the compound BD2 (dopant material (BD)) were co-deposited on the second hole transporting layer such that the ratio of the compound BD2 accounted for 2 mass %, thereby forming a 5-nm-thick first emitting layer.

The compound BH2 (second host material (BH)) and the compound BD2 (dopant material (BD)) were co-deposited on the first emitting layer so that the ratio of the compound BD2 accounted for 2 mass %, thereby forming a 20-nm-thick second emitting layer.

A compound ET9 was vapor-deposited on the second emitting layer to form a 10-nm-thick first electron transporting layer (also referred to as a hole blocking layer (HBL)).

The compound ET2 was vapor-deposited on the first electron transporting layer to form a 15-nm-thick second electron transporting layer (ET).

LiF was vapor-deposited on the second electron transporting layer to form a 1-nm-thick electron injecting layer.

Metal (Al) was vapor-deposited on the electron injecting layer to form a 50-nm-thick cathode.

A device arrangement of the organic EL device in Example 145 is roughly shown as follows.

### ITO(130)/HA3(10)/HT9(80)/HT10(10)/BH1-84:BD2(5.98%:2%)/BH2:BD2(20.98%:2%)/ET9(10)/ET2(15)/LiF(1)/Al(50)

The numerals in parentheses represent film thickness (unit: nm).

The numerals (98%:2%) represented by percentage in the same parentheses indicate a ratio (mass %) between the host material (compound BH1-84) and the compound BD2 in the first emitting layer, and the numerals (98%:2%) represented by percentage in the same parentheses indicate a ratio (mass %) between the host material (compound BH2) and the compound BD2 in the second emitting layer.

### Comparative Example 118

An organic EL device in Comparative Example 118 was manufactured in the same manner as in Example 145 except that the compound BH1-84 (first host material) in the first emitting layer was replaced by the first compound shown in Table 46.

TABLE 46

| | First Emitting Layer | | | Second Emitting Layer | | | | | |
|---|---|---|---|---|---|---|---|---|---|
| | First | Third | Film | Second | Fourth | Film | | | |
| | Com- | Com- | Thickness | Com- | Com- | Thickness | EQE | LT90 | |
| | pound | pound | [nm] | pound | pound | [nm] | [%] | [hr] | CIE-^{y} |
| Ex. 145 | BH1-84 | BD2 | 5 | BH2 | BD2 | 20 | 10.1 | 160 | 0.088 |
| Comp. | R-BH3 | BD2 | 5 | BH2 | BD2 | 20 | 9.5 | 155 | 0.090 |
| Ex. 118 | | | | | | | | | |

### Evaluation of Compounds

### Preparation of Toluene Solution

The compound BD1 was dissolved in toluene at a concentration of 4.9×10⁻⁶ mol/L to prepare a toluene solution of the compound BD1. A toluene solution of the compound BD2 and a toluene solution of the compound BD3 were prepared in the same manner.

### Measurement of Fluorescence Main Peak Wavelength (FL-Peak)

Fluorescence main peak wavelength of the toluene solution of the compound BD1 excited at 390 nm was measured using a fluorescence spectrometer (spectrophotofluorometer F-7000 (manufactured by Hitachi High-Tech Science Corporation)). The fluorescence main peak wavelengths of the toluene solutions of the compound BD2 and the compound BD3 were measured in the same manner as the compound BD1.

The fluorescence main peak wavelength of the compound BD1 was 453 nm.

The fluorescence main peak wavelength of the compound BD2 was 455 nm.

The fluorescence main peak wavelength of the compound BD3 was 451 nm.

### EXPLANATION OF CODES

1 . . . organic EL device, 2 . . . substrate, 3 . . . anode, 4 . . . cathode, 51 . . . first emitting layer, 52 . . . second emitting layer, 6 . . . hole injecting layer, 7 . . . hole transporting layer, 8 . . . electron transporting layer, 9 . . . electron injecting layer

1. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11) below, the first compound being represented by a formula (1) below,
the second emitting layer comprises as a second host material, 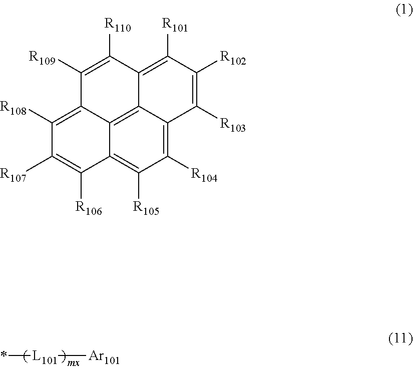
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a single bond, or a substituted or unsubstituted arylene group having 6 to 50 ring carbon atoms;
Ar₁₀₁ is a substituted or unsubstituted aryl group having 6 to 50 ring carbon atoms;
mx is 0, 1, 2, 3, 4, or 5;
Ar₁₀₁ is not a substituted or unsubstituted pyrenyl group;
L₁₀₁ is not a substituted or unsubstituted pyrenylene group;
a substituted or unsubstituted aryl group having 6 to 50 ring carbon atoms as R₁₀₁ to R₁₁₀ not being a group represented by the formula (11) is not a substituted or unsubstituted pyrenyl group;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

2. The organic electroluminescence device according to claim 1, wherein
mx is 1, 2, 3, 4, or 5, and
at least one L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group.

3. The organic electroluminescence device according to claim 1, wherein
Ar₁₀₁ is an aryl group in which four or more rings are fused.

4. The organic electroluminescence device according to claim 1, wherein
the group represented by the formula (11) is a group represented by a formula (111) below, 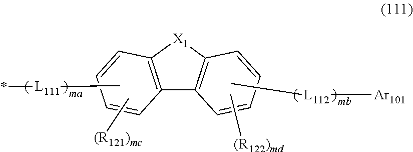
where:
X₁ is CR₁₂₃R₁₂₄;
L₁₁₁ and L₁₁₂ are each independently a single bond, or a substituted or unsubstituted arylene group having 6 to 50 ring carbon atoms;
ma is 0, 1, 2, 3, or 4;
mb is 0, 1, 2, 3, or 4;
ma+mb is 0, 1, 2, 3, or 4;
Ar₁₀₁ represents the same as Ar₁₀₁ in the formula (11);
R₁₂₁, R₁₂₂, R₁₂₃, and R₁₂₄ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, or a substituted or unsubstituted aryl group having 6 to 50 ring carbon atoms;
mc is 3;
three R₁₂₁ are mutually the same or different;
md is 3; and
three R₁₂₂ are mutually the same or different.

5. The organic electroluminescence device according to claim 4, wherein
ma is 0, 1, or 2; and mb is 0, 1, or 2.

6. The organic electroluminescence device according to claim 4, wherein
ma is 0 or 1; and mb is 0 or 1.

7. The organic electroluminescence device according to claim 1, wherein
Ar₁₀₁ is a substituted or unsubstituted phenyl group, a substituted or unsubstituted naphthyl group, a substituted or unsubstituted biphenyl group, a substituted or unsubstituted terphenyl group, a substituted or unsubstituted phenanthryl group, or a substituted or unsubstituted fluorenyl group.

8. The organic electroluminescence device according to claim 1, wherein
when Ar₁₀₁ is a substituted aryl group having 6 to 50 ring carbon atoms, a substituent of the substituted aryl group as Ar₁₀₁ is not a heterocyclic group.

9. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least two groups represented by a formula (11) below, the first compound being represented by a formula (1) below, and
the second emitting layer comprises a second compound as a second host material, 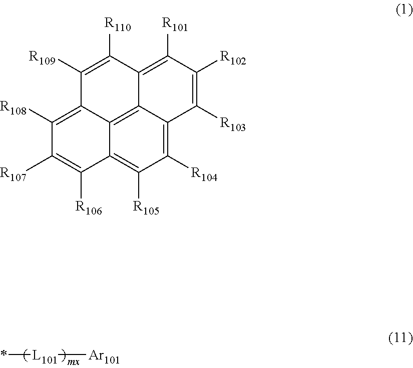
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₉₀₁, a group represented by —COOR₈₀₂, a halogen atom, a cyano group, a nitro group, or a group represented by the formula (11);
two or more of R₁₀₁ to R₁₁₀ are each a group represented by the formula (11);
when a plurality of groups represented by the formula (11) are present, the plurality of groups represented by the formula (11) are mutually the same or different;
L₁₀₁ is a single bond;
Ar₁₀₁ is a substituted or unsubstituted heterocyclic group having 5 to 50 ring atoms;
mx is 0;
when two or more Ar₁₀₁ are present, the two or more Ar₁₀₁ are mutually the same or different; and
* in the formula (11) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

10. The organic electroluminescence device according to claim 9, wherein
two or three of R₁₀₁ to R₁₁₀ are each a group represented by the formula (11).

11. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11) below, the first compound being represented by a formula (1) below, and
the second emitting layer comprises a second compound as a second host material, 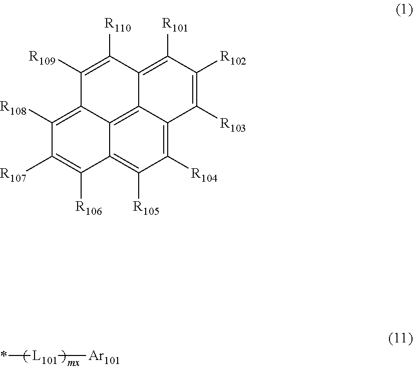
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a substituted or unsubstituted divalent heterocyclic group having 5 to 50 ring atoms;
Ar₁₀₁ is a substituted or unsubstituted heterocyclic group having 5 to 50 ring atoms;
mx is 1, 2, 3, 4, or 5;
a substituted or unsubstituted aryl group having 6 to 50 ring carbon atoms as R₁₀₁ to R₁₁₀ not being a group represented by the formula (11) is not a substituted or unsubstituted pyrenyl group;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

12. The organic electroluminescence device according to claim 11, wherein
at least one L₁₀₁ is at least one divalent heterocyclic group selected from the group consisting of a substituted or unsubstituted dibenzofurandiyl group, a substituted or unsubstituted dibenzothiophenediyl group, and a substituted or unsubstituted carbazolediyl group.

13. The organic electroluminescence device according to claim 11, wherein
Ar₁₀₁ is a heterocyclic group in which four or more rings are fused.

14. The organic electroluminescence device according to claim 13, wherein
one of R₁₀₁ to R₁₁₀ is a group represented by the formula (11).

15. The organic electroluminescence device according to claim 13, wherein
mx is 1.

16. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11) below, the first compound being represented by a formula (1) below, and
the second emitting layer comprises a second compound as a second host material, 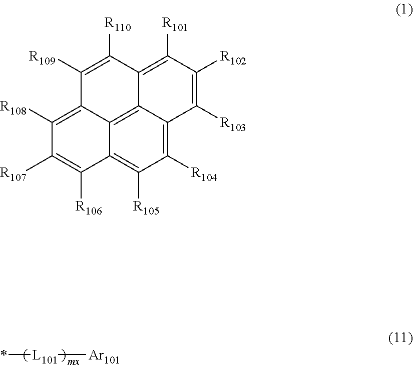
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
at least one of R₁₀₁ to R₁₁₀ is a substituted or unsubstituted heterocyclic group having 5 to 50 ring atoms;
when a plurality of groups represented by the formula (11) are present, the plurality of groups represented by the formula (11) are mutually the same or different;
L₁₀₁ is a single bond, or a substituted or unsubstituted arylene group having 6 to 50 ring carbon atoms;
Ar₁₀₁ is a substituted or unsubstituted aryl group having 6 to 50 ring carbon atoms;
mx is 0, 1, 2, 3, 4, or 5;
Ar₁₀₁ is not a substituted or unsubstituted pyrenyl group;
L₁₀₁ is not a substituted or unsubstituted pyrenylene group;
a substituted or unsubstituted aryl group having 6 to 50 ring carbon atoms as R₁₀₁ to R₁₁₀ not being a group represented by the formula (11) is not a substituted or unsubstituted pyrenyl group;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring in the formula (1),
in the first compound represented by the formula (1), R₉₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

17. The organic electroluminescence device according to claim 16, wherein
mx is 1, 2, 3, 4, or 5; and
at least one L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group.

18. The organic electroluminescence device according to claim 16, wherein
Ar₁₀₁ is an aryl group in which four or more rings are fused.

19. The organic electroluminescence device according to claim 16, wherein
a substituted or unsubstituted heterocyclic group having 5 to 50 ring atoms as R₁₀₁ to R₁₁₀ is a heterocyclic group selected from the group consisting of a monocyclic heterocyclic group, a heterocyclic group in which two rings are fused, and a heterocyclic group in which three rings are fused.

20. The organic electroluminescence device according to claim 16, wherein
one or two of R₁₀₁ to R₁₁₀ are each a group represented by the formula (11).

21. The organic electroluminescence device according to claim 16, wherein
Ar₁₀₁ is a substituted or unsubstituted phenyl group, a substituted or unsubstituted naphthyl group, a substituted or unsubstituted biphenyl group, a substituted or unsubstituted terphenyl group, a substituted or unsubstituted phenanthryl group, or a substituted or unsubstituted fluorenyl group.

22. The organic electroluminescence device according to claim 16, wherein
mx is 1, 2, 3, 4, or 5; and
L₁₀₁ is a substituted or unsubstituted arylene group having 6 to 50 ring carbon atoms.

23. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11) below, the first compound being represented by a formula (1) below, and
the second emitting layer comprises a second compound as a second host material, 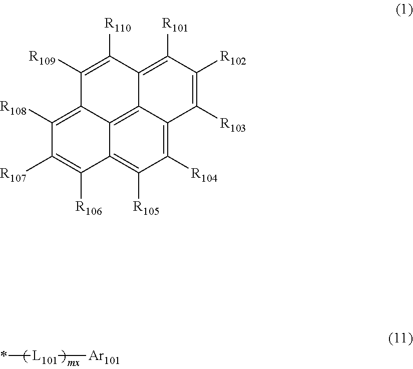
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a substituted or unsubstituted arylene group having 6 to 50 ring carbon atoms, or a substituted or unsubstituted divalent heterocyclic group having 5 to 50 ring atoms;
Ar₁₀₁ is a substituted or unsubstituted heterocyclic group having 5 to 50 ring atoms;
mx is 1, 2, 3, 4, or 5;
L₁₀₁ is not a substituted or unsubstituted pyrenylene group;
a substituted or unsubstituted aryl group having 6 to 50 ring carbon atoms as R₁₀₁ to R₁₁₀ not being a group represented by the formula (11) is not a substituted or unsubstituted pyrenyl group;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

24. The organic electroluminescence device according to claim 23, wherein
at least one L₁₀₁ is at least one arylene group selected from the group consisting of a substituted or unsubstituted phenylene group, a substituted or unsubstituted biphenylene group, a substituted or unsubstituted terphenylene group, a substituted or unsubstituted naphthylene group, a substituted or unsubstituted fluorenediyl group, and a substituted or unsubstituted phenanthrylene group.

25. The organic electroluminescence device according to claim 23, wherein
Ar₁₀₁ is a heterocyclic group in which four or more rings are fused.

26. The organic electroluminescence device according to claim 23, wherein
L₁₀₁ is a substituted or unsubstituted arylene group having 6 to 50 ring carbon atoms.

27. The organic electroluminescence device according to claim 23, wherein
one of R₁₀₁ to R₁₁₀ is a group represented by the formula (11).

28. The organic electroluminescence device according to claim 23, wherein
mx is 1.

29. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11) below, the first compound being represented by a formula (1) below, and
the second emitting layer comprises a second compound as a second host material, 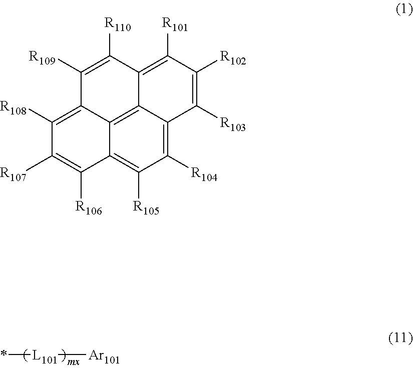
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11);
at least one of R₁₀₁ to R₁₁₀ is a group represented by the formula (11);
when a plurality of groups represented by the formula (11) are present, the plurality of groups represented by the formula (11) are mutually the same or different;
L₁₀₁ is a substituted or unsubstituted divalent heterocyclic group having 5 to 50 ring atoms;
Ar₁₀₁ is a substituted or unsubstituted aryl group having 6 to 50 ring carbon atoms;
mx is 1, 2, 3, 4, or 5;
Ar₁₀₁ is not a substituted or unsubstituted pyrenyl group;
a substituted or unsubstituted aryl group having 6 to 50 ring carbon atoms as R₁₀₁ to R₁₁₀ not being a group represented by the formula (11) is not a substituted or unsubstituted pyrenyl group;
when two or more L₁₀₁ are present, the two or more L₁₀₁ are mutually the same or different;
when two or more Ar₁₀₁ are present, the two or more Ar₁₀₁ are mutually the same or different; and
* in the formula (11) represents a bonding position to a pyrene ring in the formula (1),
in the first compound represented by the formula (1) R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

30. The organic electroluminescence device according to claim 29, wherein
at least one L₁₀₁ is at least one divalent heterocyclic group selected from the group consisting of a substituted or unsubstituted dibenzofurandiyl group, a substituted or unsubstituted dibenzothiophenediyl group, and a substituted or unsubstituted carbazolediyl group.

31. The organic electroluminescence device according to claim 29, wherein
Ar₁₀₁ is an aryl group in which four or more rings are fused.

32. The organic electroluminescence device according to claim 29, wherein
one of R₁₀₁ to R₁₁₀ is a group represented by the formula (11).

33. The organic electroluminescence device according to claim 29, wherein
mx is 1.

34. The organic electroluminescence device according to claim 29, wherein
Ar₁₀₁ is a substituted or unsubstituted phenyl group, a substituted or unsubstituted naphthyl group, a substituted or unsubstituted biphenyl group, a substituted or unsubstituted terphenyl group, a substituted or unsubstituted phenanthryl group, or a substituted or unsubstituted fluorenyl group.

35. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11A) below, the first compound being represented by a formula (1) below, and
the second emitting layer comprises a second compound as a second host material, 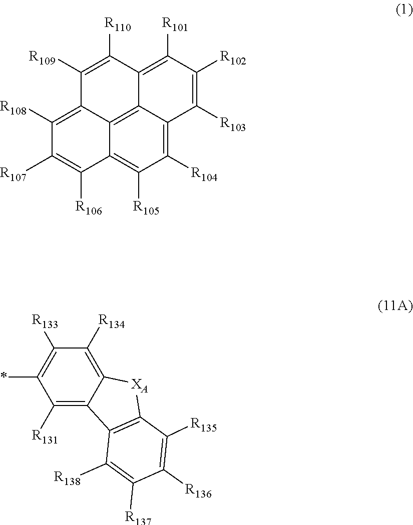
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11A);
at least one of R₁₀₁ to R₁₁₀ is a group represented by the formula (11A);
when a plurality of groups represented by the formula (11A) are present, the plurality of groups represented by the formula (11A) are mutually the same or different;
X_{A} is an oxygen atom or a sulfur atom;
at least one combination of adjacent two or more of R₁₃₁, R₁₃₃, and R₁₃₄ are mutually bonded to form a substituted or unsubstituted monocyclic ring, mutually bonded to form a substituted or unsubstituted fused ring, or not mutually bonded;
R₁₃₅ to R₁₃₈, and R₁₃₁, R₁₃₃, and R₁₃₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₉₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (11A) represents a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

36. The organic electroluminescence device according to claim 35, wherein
the first compound comprises one group represented by the formula (11A).

37. The organic electroluminescence device according to claim 35, wherein
R₁₀₁, R₁₀₃, R₁₀₆, or R₁₀₈ is a group represented by the formula (11A).

38. The organic electroluminescence device according to claim 35, wherein
R₁₃₅ to R₁₃₈ are each a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, or a nitro group.

39. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11B) below, the first compound being represented by a formula (1) below, and
the second emitting layer comprises a second compound as a second host material, 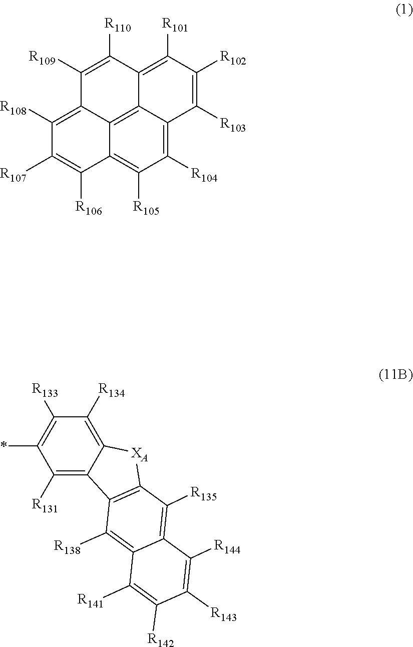
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11B);
at least one of R₁₀₁ to R₁₁₀ is a group represented by the formula (11B);
when a plurality of groups represented by the formula (11B) are present, the plurality of groups represented by the formula (11B) are mutually the same or different;
X_{A} is an oxygen atom or a sulfur atom;
at least one combination of adjacent two or more of R₁₃₁, R₁₃₃ to R₁₃₅, R₁₃₈ and R₁₄₁ to R₁₄₄ are mutually bonded to form a substituted or unsubstituted monocyclic ring, mutually bonded to form a substituted or unsubstituted fused ring, or not mutually bonded;
R₁₃₁, R₁₃₃ to R₁₃₅, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
at least one of R₁₃₅, R₁₃₈ or R₁₄₁ to R₁₄₄ is a substituted or unsubstituted aryl group having 6 to 50 ring carbon atoms, or a substituted or unsubstituted heterocyclic group having 5 to 50 ring atoms; and
* in the formula (11B) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

40. The organic electroluminescence device according to claim 39, wherein
the first compound comprises one group represented by the formula (11B).

41. The organic electroluminescence device according to claim 39, wherein
R₁₀₁, R₁₀₃, R₁₀₆, or R₁₀₈ is a group represented by the formula (11B).

42. An organic electroluminescence device comprising:
an anode;
a cathode;
a first emitting layer provided between the anode and the cathode;
a second emitting layer provided between the first emitting layer and the cathode, wherein
the first emitting layer comprises, as a first host material, a first compound that comprises at least one group represented by a formula (11C) below, the first compound being represented by a formula (1) below, and
the second emitting layer comprises a second compound as a second host material, and
the first emitting layer is in direct contact with the second emitting layer, 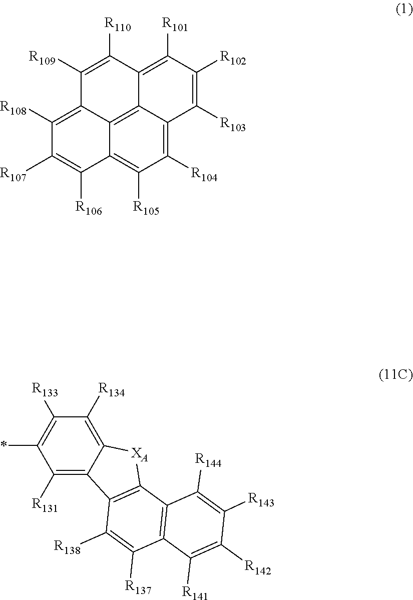
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11C);
at least one of R₁₀₁ to R₁₁₀ is a group represented by the formula (11C);
when a plurality of groups represented by the formula (11C) are present, the plurality of groups represented by the formula (11C) are mutually the same or different;
X_{A} is an oxygen atom or a sulfur atom;
at least one combination of adjacent two or more of R₁₃₁, R₁₃₃, R₁₃₄, R₁₃₇, R₁₃₈ and R₁₄₁ to R₁₄₄ are mutually bonded to form a substituted or unsubstituted monocyclic ring, mutually bonded to form a substituted or unsubstituted fused ring, or not mutually bonded;
R₁₃₁, R₁₃₃, R₁₃₄, R₁₃₇, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (11C) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

43. The organic electroluminescence device according to claim 42, wherein
the first compound comprises one group represented by the formula (11C).

44. The organic electroluminescence device according to claim 42, wherein
R₁₀₁, R₁₀₃, R₁₀₆, or R₁₀₈ is a group represented by the formula (11C).

45. An organic electroluminescence device comprising:
an anode;
a cathode;
a first emitting layer provided between the anode and the cathode; and
a second emitting layer provided between the first emitting layer and the cathode, wherein
the first emitting layer comprises, as a first host material, a first compound that comprises at least one group represented by a formula (11D) below, the first compound being represented by a formula (1) below, and
the second emitting layer comprises a second compound as a second host material, 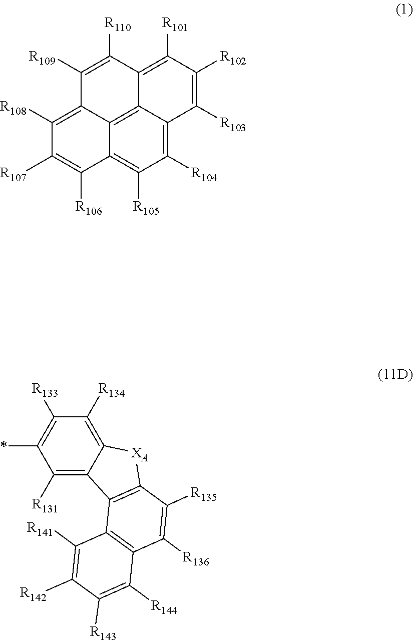
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11D);
at least one of R₁₀₁ to R₁₁₀ is a group represented by the formula (11D);
when a plurality of groups represented by the formula (11D) are present, the plurality of groups represented by the formula (11D) are mutually the same or different;
X_{A} is an oxygen atom or a sulfur atom;
at least one combination of adjacent two or more of R₁₃₁, R₁₃₃, R₁₃₄, R₁₃₅, R₁₃₆ and R₁₄₁ to R₁₄₄ are mutually bonded to form a substituted or unsubstituted monocyclic ring, mutually bonded to form a substituted or unsubstituted fused ring, or not mutually bonded;
R₁₃₁, R₁₃₃, R₁₃₄, R₁₃₅, R₁₃₆ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
* in the formula (11D) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

46. The organic electroluminescence device according to claim 45, wherein
the first compound comprises one group represented by the formula (11D).

47. The organic electroluminescence device according to claim 45, wherein
R₁₀₁, R₁₀₃, R₁₀₆, or R₁₀₈ is a group represented by the formula (11D).

48. An organic electroluminescence device comprising:
an anode;
a cathode;
a first emitting layer provided between the anode and the cathode;
a second emitting layer provided between the first emitting layer and the cathode, wherein
the first emitting layer comprises, as a first host material, a first compound that comprises at least one group represented by a formula (11E) below, the first compound being represented by a formula (1) below, and
the second emitting layer comprises a second compound as a second host material, 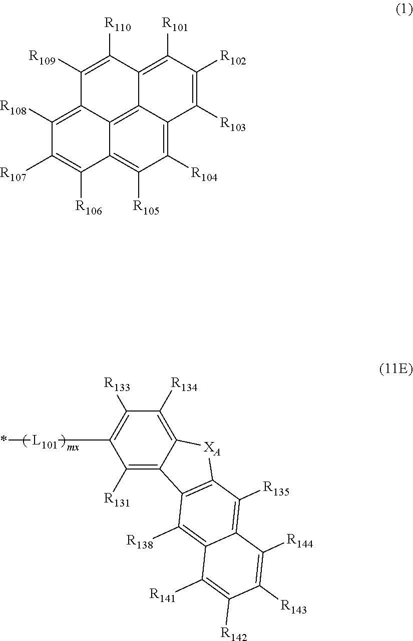
where:
R₁₀₁ to R₁₁₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by the formula (11E);
at least one of R₁₀₁ to R₁₁₀ is a group represented by the formula (11E);
when a plurality of groups represented by the formula (11E) are present, the plurality of groups represented by the formula (11E) are mutually the same or different;
X_{A} is an oxygen atom or a sulfur atom;
at least one combination of adjacent two or more of R₁₃₁, R₁₃₃ to R₁₃₅, R₁₃₈ and R₁₄₁ to R₁₄₄ are mutually bonded to form a substituted or unsubstituted monocyclic ring, mutually bonded to form a substituted or unsubstituted fused ring, or not mutually bonded;
R₁₃₁, R₁₃₃ to R₁₃₅, R₁₃₈ and R₁₄₁ to R₁₄₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₁₀₁ is a substituted or unsubstituted arylene group having 6 to 50 ring carbon atoms, or a substituted or unsubstituted divalent heterocyclic group having 5 to 50 ring atoms;
mx is 1, 2, 3, 4, or 5;
when two or more L₁₀₁ are present, the two or more L₁₀₁ are mutually the same or different; and
* in the formula (11E) represents a bonding position to a pyrene ring in the formula (1),
in the first compound represented by the formula (1), R₉₀₁, R₉₀₂, R₉₀₃, R₉₀₄, R₉₀₅, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

49. The organic electroluminescence device according to claim 48, wherein
the first compound comprises one group represented by the formula (11E).

50. The organic electroluminescence device according to claim 48, wherein
R₁₀₁, R₁₀₃, R₁₀₆, or R₁₀₈ is a group represented by the formula (11E).

51. The organic electroluminescence device according to claim 48, wherein
the group represented by the formula (11) is a group represented by a formula (111E) below, 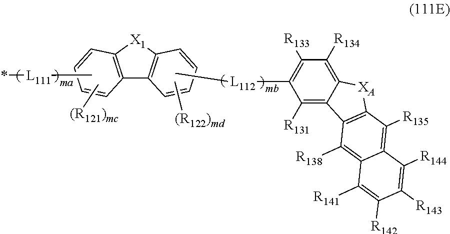
where:
X₁ is CR₁₂₃R₁₂₄, an oxygen atom, a sulfur atom, or NR₁₂₅;
L₁₁₁ and L₁₁₂ are each independently a single bond, a substituted or unsubstituted arylene group having 6 to 50 ring carbon atoms, or a substituted or unsubstituted divalent heterocyclic group having 5 to 50 ring atoms;
ma is 1, 2, 3, or 4;
mb is 1, 2, 3, or 4;
ma+mb is 2, 3, or 4;
X_{A}, R₁₃₁, R₁₃₃ to R₁₃₅, R₁₃₈ and R₁₄₁ to R₁₄₄ each represent the same as X_{A}, R₁₃₁, R₁₃₃ to R₁₃₅, R₁₃₈ and R₁₄₁ to R₁₄₄ in the formula (11E);
R₁₂₁, R₁₂₂, R₁₂₃, R₁₂₄ and R₁₂₅ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
mc is 3;
three R₁₂₁ are mutually the same or different;
md is 3; and
three R₁₂₂ are mutually the same or different.

52. The organic electroluminescence device according to claim 51, wherein
ma is 1 or 2; and mb is 1 or 2.

53. The organic electroluminescence device according to claim 51, wherein
ma is 1, and mb is 1.

54. The organic electroluminescence device according to claim 35, wherein
R₁₀₁ to R₁₁₀ not being a group represented by the formula (11A) are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

55-57. (canceled)

58. The organic electroluminescence device according to claim 1, wherein
R₁₀₁ to R₁₁₀ not being a group represented by the formula (11) are each independently a hydrogen atom, a substituted or unsubstituted alkyl group having 1 to 50 carbon atoms, or a substituted or unsubstituted cycloalkyl group having 3 to 50 ring carbon atoms.

59. The organic electroluminescence device according to claim 1, wherein
R₁₀₁ to R₁₁₀ not being a group represented by the formula (11) are each a hydrogen atom.

60-65. (canceled)

66. The organic electroluminescence device according to claim 1, wherein
all groups defined to be substituted or unsubstituted in the first compound are unsubstituted groups.

67-69. (canceled)

70. The organic electroluminescence device according to claim 1, wherein the second compound is represented by a formula (2) below, 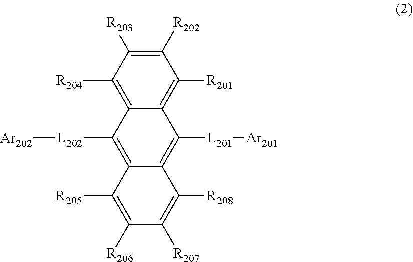
where:
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms;
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms;
in the second compound represented by the formula (2), R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

71. The organic electroluminescence device according to claim 1, wherein
the first emitting layer is in direct contact with the second emitting layer.
